(19) United States
(12) Patent Application Publication
(10) Pub. No.: US 2025/0034201 A1
(43) Pub. Date: Jan. 30, 2025

(54) PRODRUGS OF GANAXOLONE

(71) Applicant: MARINUS PHARMACEUTICALS, INC., Radnor, PA (US)

(72) Inventors: Jay E. WROBEL, Lawrenceville, NJ (US); Justin Barrett SAUSKER, Doylestown, PA (US); Allen B. REITZ, Lansdale, PA (US); Colin M. TICE, Ambler, PA (US); Randall Jeffrey BINDER, Philadelphia, PA (US)

(21) Appl. No.: 18/825,194

(22) Filed: Sep. 5, 2024

Related U.S. Application Data
(63) Continuation of application No. PCT/US2023/064615, filed on Mar. 17, 2023.
(60) Provisional application No. 63/321,302, filed on Mar. 18, 2022.
(60) Provisional application No. 63/392,878, filed on Jul. 28, 2022.
(60) Provisional application No. 63/485,657, filed on Feb. 17, 2023.

(51) Int. Cl. C07J 43/00; A61K 31/57; A61K 31/58; C07J 7/00; C07J 31/00; C07J 41/00

(52) U.S. Cl. CPC C07J 43/003; A61K 31/57; A61K 31/58; C07J 7/002; C07J 31/006; C07J 41/005; C07J 41/0088

(57) ABSTRACT

This disclosure relates to ganaxolone derivatives, as well as methods of using the compounds (e.g., for treatment of a disease or disorder), methods of making the compounds, and pharmaceutical compositions and kits comprising the same.

### BACKGROUND

Ganaxolone is a positive allosteric modulator of gamma-aminobutyric acid (GABA) receptors (GABA_{A}), and can increase GABA activity and inhibit neuronal excitability. Ganaxolone has a very low aqueous solubility which greatly reduces its oral bioavailability and limits its potential clinical use.

Prodrugs are molecules with typically little or no pharmacological activity of their own, that can be metabolized to release a pharmacologically active drug (i.e., parent drug) following administration (e.g., in the blood). Some prodrugs can be absorbed more readily, and have superior oral bioavailability, relative to the parent drug. However, success in creating a stable prodrug with superior bioavailability relative to a parent drug and suitable conversion to the parent drug and safety (e.g., toxicity profile) is unpredictable.

As such, there is an unmet need for a ganaxolone prodrug.

### SUMMARY

The present disclosure relates to ganaxolone derivatives. The compounds disclosed herein may be metabolized to ganaxolone (e.g., in vivo, e.g., by a metabolic process). Compounds disclosed herein are also more soluble in aqueous solutions such as plasma, gastric fluid, or intestinal fluid, than ganaxolone. Also disclosed herein are methods of using the compounds, methods of making the compounds, and pharmaceutical compositions and kits comprising the same.

A compound of the present disclosure may be a compound of Formula (I):

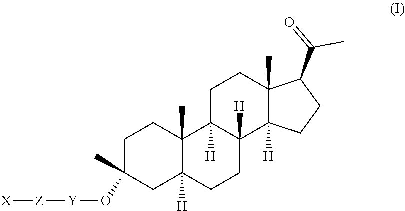

or a pharmaceutically acceptable salt, solvate, hydrate, tautomer, or stereoisomer thereof, wherein Y is —C(O)— or —C(R^{A})(R^{B})— (e.g., —CH₂—);
Z is absent, alkylene, alkenylene, alkynylene, heteroalkylene, cycloalkylene, heterocyclylene, arylene, heteroarylene, —NR—, —O—, or —S—, wherein the alkylene, alkenylene, alkynylene, or heteroalkylene is optionally substituted;
X is alkyl, alkenyl, alkynyl, heteroalkyl, cycloalkyl, heterocyclyl, aryl, heteroaryl, haloalkyl, alkylene-cycloalkyl, alkylene-heterocyclyl, alkylene-aryl, alkylene-heteroaryl, heteroalkylene-cycloalkyl, heteroalkylene-heterocyclyl, heteroalkylene-aryl, heteroalkylene-heteroaryl, cycloalkylene-alkyl, cycloalkylene-heteroalkyl, heterocyclylene-alkyl, heterocyclylene-heteroalkyl, —OC(O)-alkyl, —C(O)O-alkyl, —OC(O)-heteroalkyl, —C(O)O— heteroalkyl, —N₃, —NR¹R², —OR³, or —SR⁴, wherein the alkyl, alkylene, alkenyl, alkynyl, heteroalkyl, heteroalkylene, cycloalkyl, cycloalkylene, heterocyclyl, heterocyclylene, aryl, or heteroaryl is optionally substituted;
R¹ and R² are each independently hydrogen, alkyl, alkenyl, alkynyl, heteroalkyl, cycloalkyl, heterocyclyl, aryl, heteroaryl, alkylene-cycloalkyl, alkylene-heterocyclyl, alkylene-aryl, alkylene-heteroaryl, heteroalkylene-cycloalkyl, heteroalkylene-heterocyclyl, heteroalkylene-aryl, heteroalkylene-heteroaryl, cycloalkylene-alkyl, cycloalkylene-heteroalkyl, heterocyclylene-alkyl, or heterocyclylene-heteroalkyl, wherein the alkyl, alkylene, alkenyl, alkynyl, heteroalkyl, heteroalkylene, cycloalkyl, cycloalkylene, heterocyclyl, heterocyclylene, aryl, or heteroaryl is optionally substituted;
or R¹ and R², together with the atoms to which they are attached, form a 3-7 membered cycloalkyl, heterocyclyl, aryl, or heteroaryl, wherein the cycloalkyl, heterocyclyl, aryl, or heteroaryl is optionally substituted;
R³ is hydrogen, alkyl, alkenyl, alkynyl, heteroalkyl, cycloalkyl, heterocyclyl, aryl, heteroaryl, alkylene-cycloalkyl, alkylene-heterocyclyl, alkylene-aryl, alkylene-heteroaryl, heteroalkylene-cycloalkyl, heteroalkylene-heterocyclyl, heteroalkylene-aryl, heteroalkylene-heteroaryl, cycloalkylene-alkyl, cycloalkylene-heteroalkyl, heterocyclylene-alkyl, or heterocyclylene-heteroalkyl, wherein the alkyl, alkylene, alkenyl, alkynyl, heteroalkyl, heteroalkylene, cycloalkyl, cycloalkylene, heterocyclyl, heterocyclylene, aryl, or heteroaryl is optionally substituted;
R⁴ is hydrogen, alkyl, alkenyl, alkynyl, heteroalkyl, cycloalkyl, heterocyclyl, aryl, heteroaryl, alkylene-cycloalkyl, alkylene-heterocyclyl, alkylene-aryl, alkylene-heteroaryl, heteroalkylene-cycloalkyl, heteroalkylene-heterocyclyl, heteroalkylene-aryl, heteroalkylene-heteroaryl, wherein the alkyl, alkylene, alkenyl, alkynyl, heteroalkyl, heteroalkylene, cycloalkyl, cycloalkylene, heterocyclyl, heterocyclylene, aryl, or heteroaryl is optionally substituted; and
R^{A} and R^{B} are each independently hydrogen, alkyl, heteroalkyl, cycloalkyl, heterocyclyl, aryl, or heteroaryl, wherein the alkyl, heteroalkyl, cycloalkyl, heterocyclyl, aryl, or heteroaryl is optionally substituted.

For example, the compound may be a compound of Formula (I), wherein Y is —C(O)—.

The compound may be a compound of Formula (I), wherein Z is methylene (e.g., unsubstituted methylene),

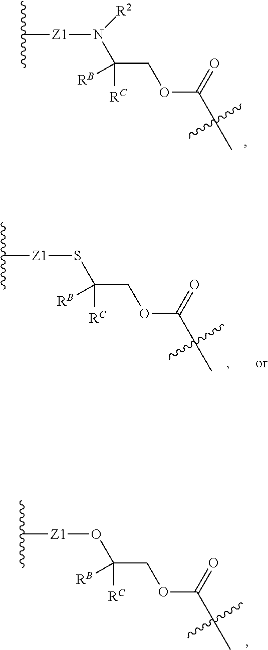

wherein Z1 is absent, alkylene (e.g., methylene, e.g., unsubstituted methylene), alkenylene, alkynylene, heteroalkylene, cycloalkylene, heterocyclylene, arylene, or heteroarylene, wherein the alkylene, alkenylene, alkynylene, or heteroalkylene is optionally substituted; R² is hydrogen or alkyl; R^{B} and R^{C} are each independently hydrogen, alkyl, heteroalkyl, cycloalkyl, heterocyclyl, aryl, or heteroaryl, wherein the alkyl, heteroalkyl, cycloalkyl, heterocyclyl, aryl, or heteroaryl is optionally substituted; and each [symbol] denotes the point of attachment of Z to X or Y.

A compound of the present disclosure may be a compound of Formula (I-I):

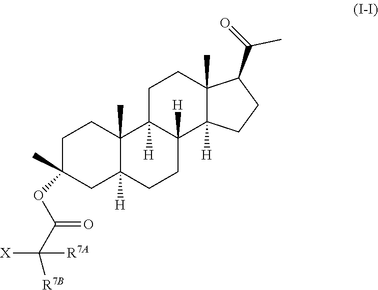

or a pharmaceutically acceptable salt, solvate, hydrate, tautomer, or stereoisomer thereof, wherein:
X is as defined herein, e.g., alkyl, alkenyl, alkynyl, heteroalkyl, cycloalkyl, heterocyclyl, aryl, heteroaryl, haloalkyl, alkylene-cycloalkyl, alkylene-heterocyclyl, alkylene-aryl, alkylene-heteroaryl, heteroalkylene-cycloalkyl, heteroalkylene-heterocyclyl, heteroalkylene-aryl, heteroalkylene-heteroaryl, cycloalkylene-alkyl, cycloalkylene-heteroalkyl, heterocyclylene-alkyl, heterocyclylene-heteroalkyl, —OC(O)-alkyl, —C(O)O-alkyl, —OC(O)-heteroalkyl, —C(O)O— heteroalkyl, —N₃, —NR¹R², —OR³, or —SR⁴, wherein the alkyl, alkylene, alkenyl, alkynyl, heteroalkyl, heteroalkylene, cycloalkyl, cycloalkylene, heterocyclyl, heterocyclylene, aryl, or heteroaryl is optionally substituted;
R¹ and R² are each independently hydrogen, alkyl, alkenyl, alkynyl, heteroalkyl, cycloalkyl, heterocyclyl, aryl, heteroaryl, alkylene-cycloalkyl, alkylene-heterocyclyl, alkylene-aryl, alkylene-heteroaryl, heteroalkylene-cycloalkyl, heteroalkylene-heterocyclyl, heteroalkylene-aryl, heteroalkylene-heteroaryl, cycloalkylene-alkyl, cycloalkylene-heteroalkyl, heterocyclylene-alkyl, or heterocyclylene-heteroalkyl, wherein the alkyl, alkylene, alkenyl, alkynyl, heteroalkyl, heteroalkylene, cycloalkyl, cycloalkylene, heterocyclyl, heterocyclylene, aryl, or heteroaryl is optionally substituted;
or R¹ and R², together with the atoms to which they are attached, form a 3-7 membered cycloalkyl, heterocyclyl, aryl, or heteroaryl, wherein the cycloalkyl, heterocyclyl, aryl, or heteroaryl is optionally substituted;
R³ is hydrogen, alkyl, alkenyl, alkynyl, heteroalkyl, cycloalkyl, heterocyclyl, aryl, heteroaryl, alkylene-cycloalkyl, alkylene-heterocyclyl, alkylene-aryl, alkylene-heteroaryl, heteroalkylene-cycloalkyl, heteroalkylene-heterocyclyl, heteroalkylene-aryl, heteroalkylene-heteroaryl, cycloalkylene-alkyl, cycloalkylene-heteroalkyl, heterocyclylene-alkyl, or heterocyclylene-heteroalkyl, wherein the alkyl, alkylene, alkenyl, alkynyl, heteroalkyl, heteroalkylene, cycloalkyl, cycloalkylene, heterocyclyl, heterocyclylene, aryl, or heteroaryl is optionally substituted;
R⁴ is hydrogen, alkyl, alkenyl, alkynyl, heteroalkyl, cycloalkyl, heterocyclyl, aryl, heteroaryl, alkylene-cycloalkyl, alkylene-heterocyclyl, alkylene-aryl, alkylene-heteroaryl, heteroalkylene-cycloalkyl, heteroalkylene-heterocyclyl, heteroalkylene-aryl, heteroalkylene-heteroaryl, wherein the alkyl, alkylene, alkenyl, alkynyl, heteroalkyl, heteroalkylene, cycloalkyl, cycloalkylene, heterocyclyl, heterocyclylene, aryl, or heteroaryl is optionally substituted; and
R^{7A} and R^{7B} are each independently hydrogen or optionally substituted alkyl.

The compound may be a compound of Formula (I) or (I-I), wherein X is a moiety selected from the group consisting of

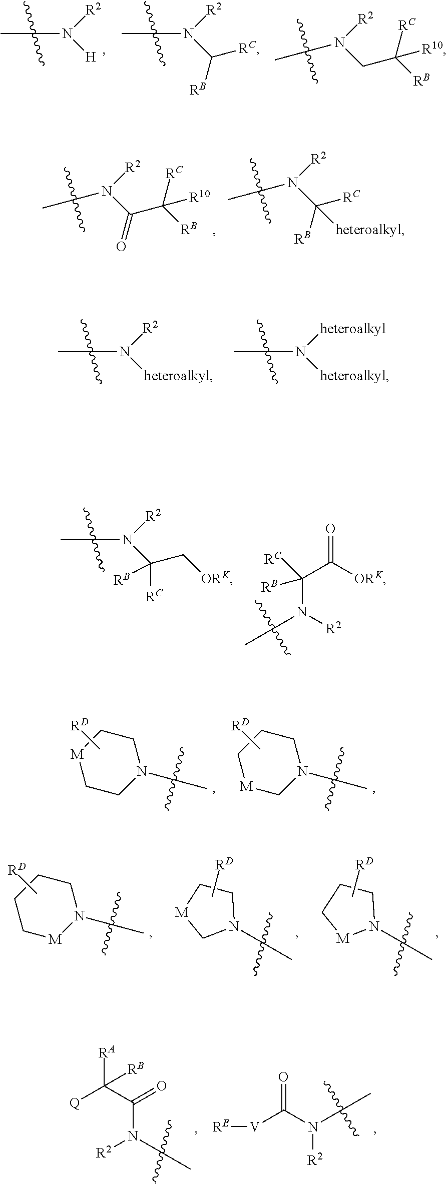

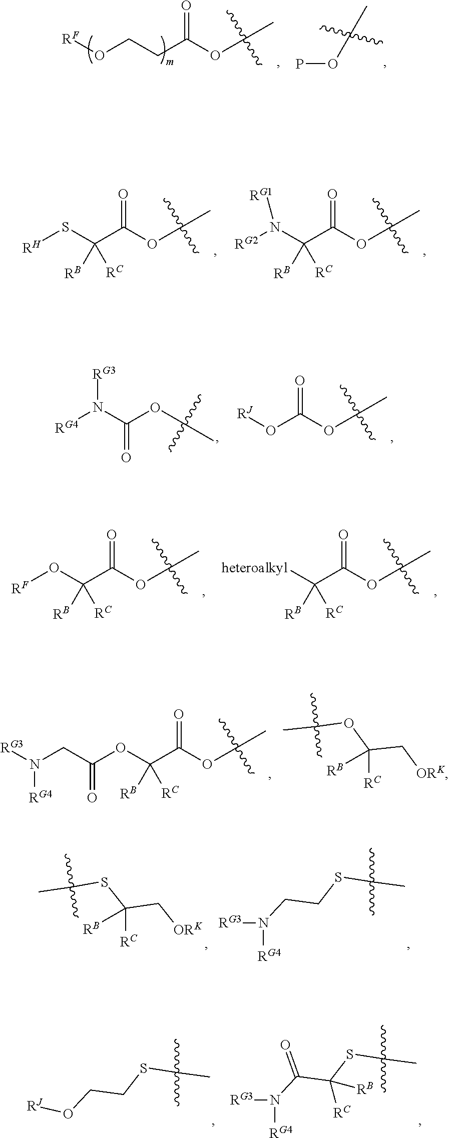

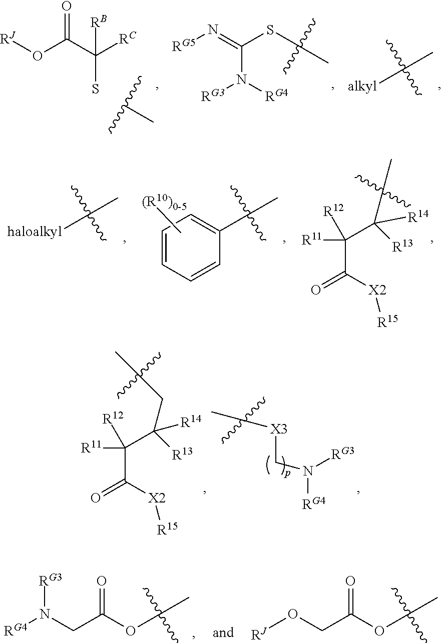

wherein
R² is hydrogen or alkyl;
M is —C(H)(R^{D})— (e.g., —CH₂—), —O—, or —N(R^{D})—(e.g., —N(Me)—);
P is an amino acid residue (e.g., glycine, alanine, valine, leucine, or proline), that may be a D-amino acid residue or L-amino acid residue, and is optionally substituted (e.g., at a terminal nitrogen atom, e.g., by a carbonate group, e.g., a carboxybenzyl (Cbz) group),
or P is a dipeptide moiety (e.g., a leucine-leucine moiety) that may comprise D-amino acids, L-amino acids, or a combination thereof, and is optionally substituted (e.g., at one or more nitrogen atoms, e.g., at a terminal nitrogen atom, e.g., by a carbonate group, e.g., a carboxybenzyl (Cbz) group);
Q is heteroalkyl or heterocyclyl, wherein the heteroalkyl or heterocyclyl is optionally substituted;
V is —O—, —N(H)—, or —N(Me)—;
X2 and X3 are each independently —CH₂—, —O—, —S—, or —N(R^{G3})— (e.g., —NH—);
R^{A}, R^{B}, R^{C}, R^{D}, R^{E}, and R^{K} are each independently hydrogen, alkyl, heteroalkyl, cycloalkyl, heterocyclyl, aryl, or heteroaryl, wherein the alkyl, heteroalkyl, cycloalkyl, heterocyclyl, aryl, or heteroaryl is optionally substituted;
R^{F} and R^{H} are each independently hydrogen or alkyl;
R^{G1} and R^{G2} are each independently hydrogen, alkyl, or an amino acid residue (e.g., glycine, alanine, valine, leucine or proline), that may be a D-amino acid residue or L-amino acid residue, and is optionally substituted (e.g., at a terminal nitrogen atom, e.g., by a carbonate group, e.g., a carboxybenzyl (Cbz) group),
or R^{G1} and R^{G2}, together with the atoms to which they are attached, form a 3-7 membered cycloalkyl, heterocyclyl, aryl, or heteroaryl, wherein the cycloalkyl, heterocyclyl, aryl, or heteroaryl is optionally substituted;
R^{G3} and R^{G4} are each independently hydrogen, alkyl, or heteroalkyl, wherein the alkyl or heteroalkyl is optionally substituted;
or R^{G3} and R^{G4}, together with the atoms to which they are attached, form a 3-7 membered cycloalkyl, heterocyclyl, aryl, or heteroaryl, wherein the cycloalkyl, heterocyclyl, aryl, or heteroaryl is optionally substituted;
R^{G5} is hydrogen, alkyl, or heteroalkyl, wherein the alkyl or heteroalkyl is optionally substituted;
R^{J} is hydrogen, alkyl, alkylene-heterocyclyl, heteroalkyl, or heterocyclyl, wherein the alkyl, alkylene, heteroalkyl, or heterocyclyl is optionally substituted;
R¹⁰ is alkyl, alkenyl, alkynyl, haloalkyl, halo, —OR^{J}, —N(R^{G3})(R^{G4}); —NO₂, —CN, or —NC;
R¹¹, R¹², R¹³, and R¹⁴ are each independently hydrogen, alkyl, alkenyl, alkynyl, heteroalkyl, haloalkyl, halo, or —N(R^{G3})(R^{G4}), wherein the alkyl, alkenyl, alkynyl, heteroalkyl, or
haloalkyl is optionally substituted,
or one of R¹¹ and R¹², and one of R¹³ and R¹⁴, together with the atoms to which they are attached, form a 3-7 membered cycloalkyl, heterocyclyl, aryl, or heteroaryl, wherein the cycloalkyl, heterocyclyl, aryl, or heteroaryl is optionally substituted;
R¹⁵ is hydrogen, alkyl, alkenyl, alkynyl, heteroalkyl, haloalkyl, or halo; wherein the alkyl, alkenyl, alkynyl, heteroalkyl, or haloalkyl is optionally substituted;
m is an integer of 1-5;
p is an integer between 0 and 4; and
[symbol] in Formula (I) denotes the point of attachment of X to Z, or X to Y when Z is absent, and [symbol] in Formula (I-I) denotes the point of attachment of X to the carbon atom of the —C(R^{7A})(R^{7B}) group.

For example, X may be

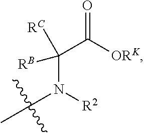

wherein R² is H, R^{B} and R^{C} are each independently alkyl (e.g., methyl), and R^{K} is hydrogen.

The compound may be a compound of Formula (I), wherein Y is —C(O)— and Z is alkylene (e.g., methylene). The compound may be a compound of Formula (I) or (I-I), wherein R¹ and R² are not both hydrogen.

The compound may be a compound of Formula (I), wherein Y is C(O)— and Z is alkylene (e.g., methylene), and X is

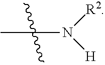

For example, the compound may be a compound of Formula (I), wherein Y is C(O)— and Z is methylene, X is

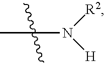

and R² is hydrogen.

The compound may be a compound of Formula (I), wherein Y is —C(O)— and Z is alkylene (e.g., methylene), and X is

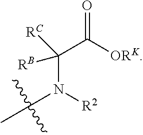

For example, the compound may be a compound of Formula (I), wherein Y is C(O)— and Z is methylene, and X is

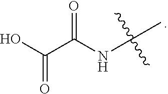 [SMILES: *NC(=O)C(=O)O]

Alternatively, the compound may be a compound of Formula (I), wherein Y is —C(O)—and Z is alkylene (e.g., methylene), and X is

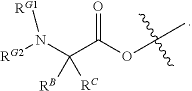

For example, the compound may be a compound of Formula (I), wherein Y is —C(O)— and Z is alkylene (e.g., methylene), and X is

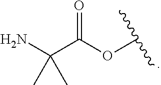 [SMILES: *OC(=O)C(C)(C)N]

A compound of the present disclosure may be a compound of Formula (I-II):

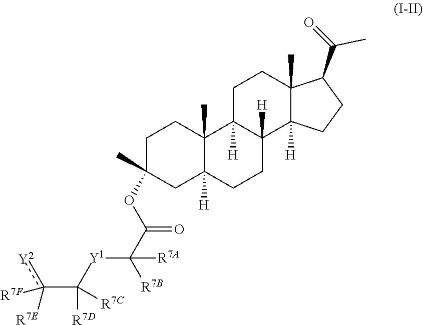

or a pharmaceutically acceptable salt, solvate, hydrate, tautomer, or stereoisomer thereof, wherein:
[symbol] denotes a single bond or a double bond;
Y¹ is alkylene (e.g., methylene), alkenylene, alkynylene, heteroalkylene, —C(O)—, —OC(O)—, —C(O)O—, —NR¹—, —O—, or —S—, wherein the alkylene, alkenylene, alkynylene, or heteroalkylene is optionally substituted;
Y² is heteroalkyl, heterocyclyl, heteroaryl, heteroalkylene-cycloalkyl, heteroalkylene-heterocyclyl, heteroalkylene-aryl, heteroalkylene-heteroaryl, —NR¹R², —OR³, —SR⁴, ═NR¹, or ═O, wherein the heteroalkyl, cycloalkyl, heterocyclyl, aryl, or heteroaryl is optionally substituted;
R¹ and R² are each independently hydrogen, alkyl, alkenyl, alkynyl, heteroalkyl, cycloalkyl, heterocyclyl, aryl, heteroaryl, alkylene-cycloalkyl, alkylene-heterocyclyl, alkylene-aryl, alkylene-heteroaryl, heteroalkylene-cycloalkyl, heteroalkylene-heterocyclyl, heteroalkylene-aryl, heteroalkylene-heteroaryl, cycloalkylene-alkyl, cycloalkylene-heteroalkyl, heterocyclylene-alkyl, or heterocyclylene-heteroalkyl, wherein the alkyl, alkylene, alkenyl, alkynyl, heteroalkyl, heteroalkylene, cycloalkyl, cycloalkylene, heterocyclyl, heterocyclylene, aryl, or heteroaryl is optionally substituted;
or R¹ and R², together with the atoms to which they are attached, form a 3-7 membered cycloalkyl, heterocyclyl, aryl, or heteroaryl, wherein the cycloalkyl, heterocyclyl, aryl, or heteroaryl is optionally substituted;
R³ is hydrogen, alkyl, alkenyl, alkynyl, heteroalkyl, cycloalkyl, heterocyclyl, aryl, heteroaryl, alkylene-cycloalkyl, alkylene-heterocyclyl, alkylene-aryl, alkylene-heteroaryl, heteroalkylene-cycloalkyl, heteroalkylene-heterocyclyl, heteroalkylene-aryl, heteroalkylene-heteroaryl, cycloalkylene-alkyl, cycloalkylene-heteroalkyl, heterocyclylene-alkyl, or heterocyclylene-heteroalkyl, wherein the alkyl, alkylene, alkenyl, alkynyl, heteroalkyl, heteroalkylene, cycloalkyl, cycloalkylene, heterocyclyl, heterocyclylene, aryl, or heteroaryl is optionally substituted;
R⁴ is hydrogen, alkyl, alkenyl, alkynyl, heteroalkyl, cycloalkyl, heterocyclyl, aryl, heteroaryl, alkylene-cycloalkyl, alkylene-heterocyclyl, alkylene-aryl, alkylene-heteroaryl, heteroalkylene-cycloalkyl, heteroalkylene-heterocyclyl, heteroalkylene-aryl, heteroalkylene-heteroaryl, wherein the alkyl, alkylene, alkenyl, alkynyl, heteroalkyl, heteroalkylene, cycloalkyl, cycloalkylene, heterocyclyl, heterocyclylene, aryl, or heteroaryl is optionally substituted;
R^{7A} and R^{7B} are each independently hydrogen or optionally substituted alkyl, or one of R^{7A} and R^{7B} is bonded to Y¹ through one or more carbon atoms or heteroatoms to form a cyclic moiety (e.g., cycloalkyl, heterocyclyl, aryl, or heteroaryl, e.g., pyrrole), and the other of the R^{7A} and R^{7B} groups is hydrogen or optionally substituted alkyl;
R^{7C} and R^{7D} are each independently hydrogen or optionally substituted alkyl, or R^{7C} and R^{7D} are each bonded to the same oxygen atom to form an oxo group; and
R^{7E} and R^{7F} are each independently absent, hydrogen, alkyl, heteroalkyl, —NR¹R², —OR³, or —SR⁴, wherein the alkyl or heteroalkyl is optionally substituted, or R^{7E} and R^{7F} are each bonded to the same oxygen to form an oxo group, with the proviso that when [symbol] is a double bond, one of R^{7E} and R^{7F} is absent.

Y¹ of Formula (I-II) may be —O— or —N(R¹)— (e.g., —N(H)—, —N(alkyl)-, or —N(heteroalkyl)-). For example, Y¹ of Formula (I-II) may be —O— or —N(H)—. Alternatively, Y¹ of Formula (I-II) may be —N(Me)—, —N(Et)-, —N(EtOH)—, or —N(ⁿPrOH)—.

The compound of Formula (I-II) may be a compound of Formula (I-IIa):

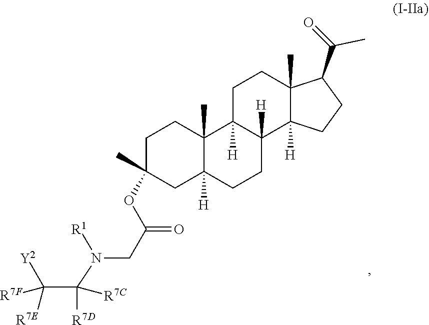

wherein Y², R¹, R^{7C}, R^{7D}, R^{7E}, and R^{7F} are as defined above; or the compound of Formula (I-I) may be a compound of Formula (I-IIb):

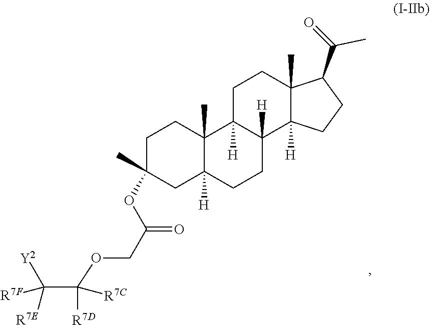

wherein Y², R^{7C}, R^{7D}, R^{7E}, and R^{7F} are as defined above; or the compound of Formula (I-II) may be a compound of Formula (I-IIc):

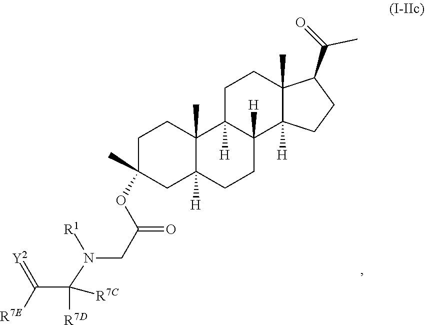

wherein Y², R¹, R^{7C}, R^{7D}, and R^{7E} are as defined above; or the compound of Formula (I-II) may be a compound of Formula (I-IId):

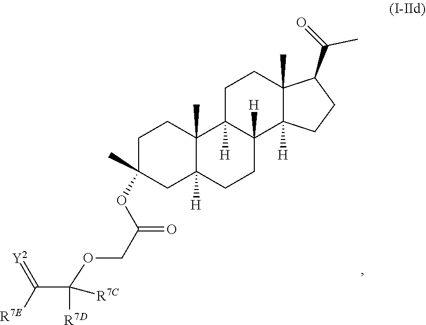

wherein Y², R^{7C}, R^{7D}, and R^{7E} are as defined above; or the compound of Formula (I-II) may be a compound of Formula (I-IIe):

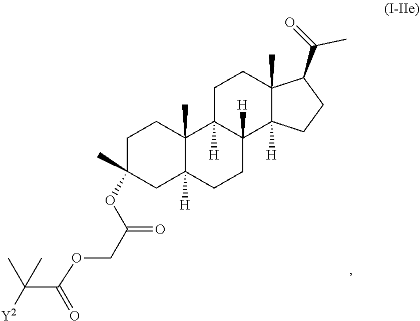

wherein Y² is as defined above.

Y² of Formulae (I-II), (I-IIa), (I-IIb), (I-IIc), (I-IId), or (I-IIe), or R^{7E} when [symbol] is a double bond, may be optionally substituted heteroalkyl, —NR¹R² (e.g., —NH₂, —NHMe, or —NMe₂), —OR³ (e.g., —OH, —OMe, —OEt, or —OtBu), or ═O. For example, Y² of Formulae (I-II), (I-IIa), (I-IIb), (I-IIc), (I-IId), or (I-IIe), or R^{7E} when [symbol] is a double bond, may be —NH₂, —NHMe, or —OH. Alternatively, Y² of Formulae (I-II), (I-IIa), (I-IIb), (I-IIc), (I-IId), or (I-IIe), or R^{7E} when [symbol] is a double bond, may be optionally substituted heteroalkyl, for example a substituted or unsubstituted heteroalkyl selected from the group consisting of:

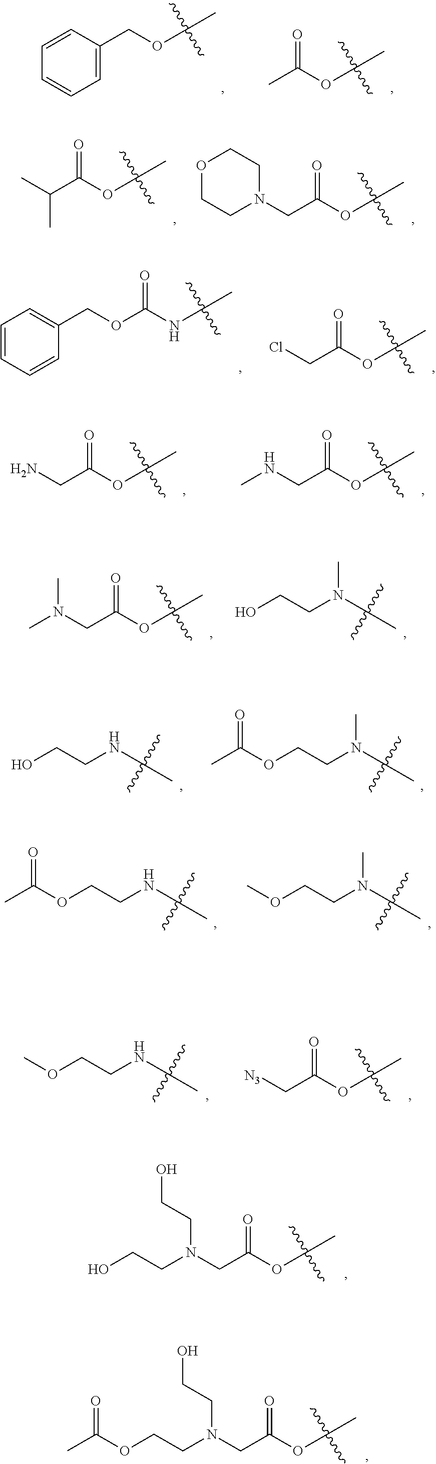

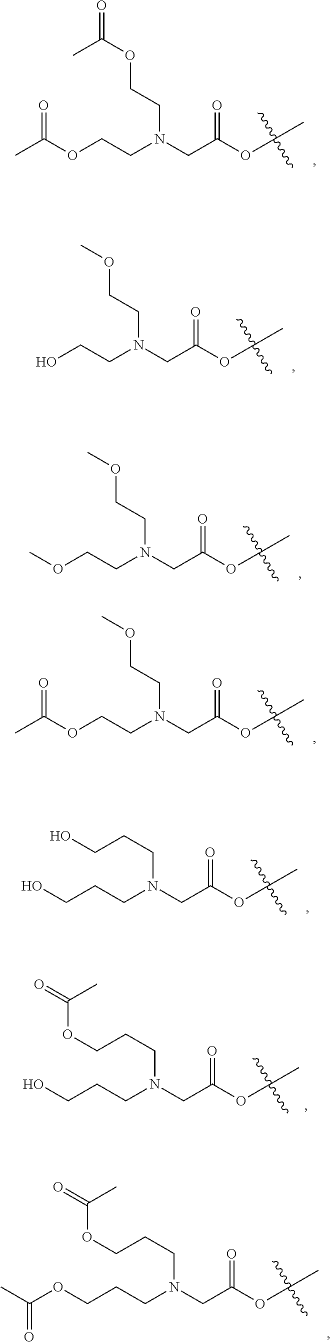

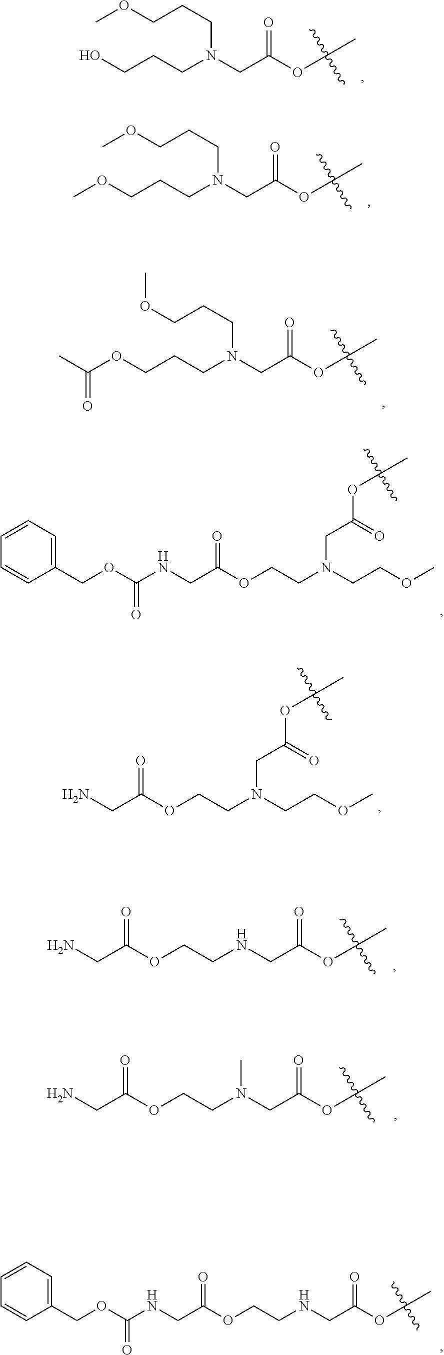

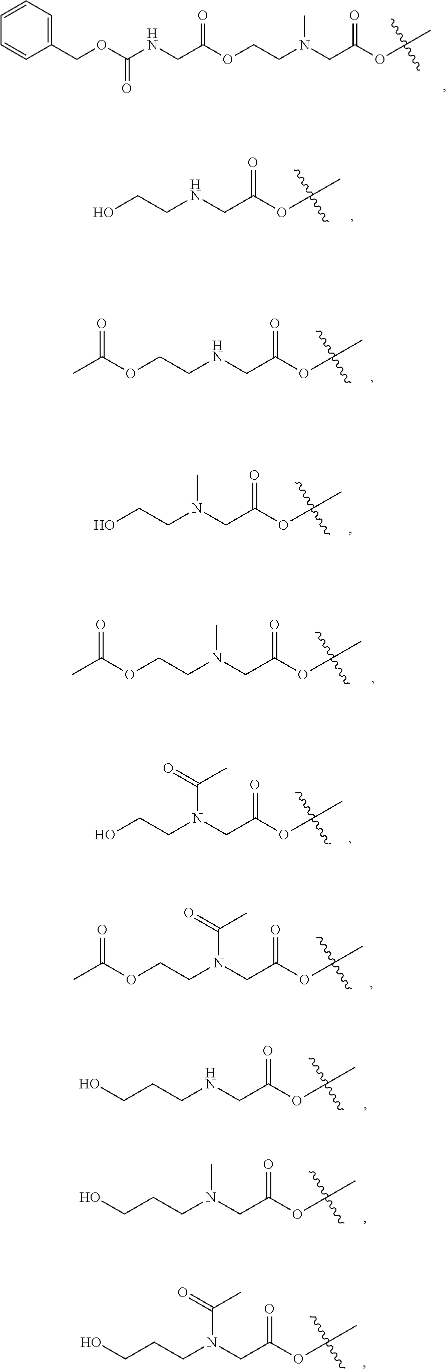

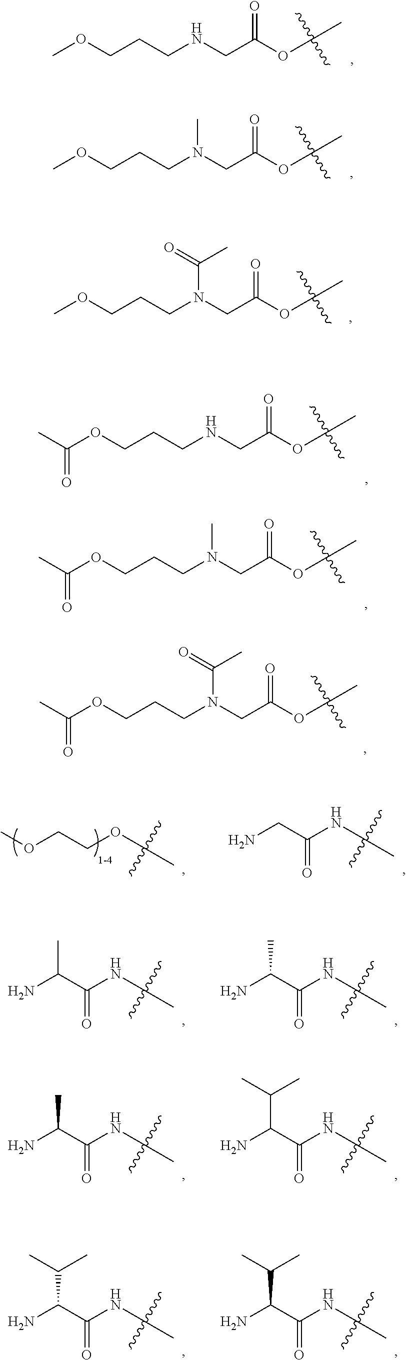

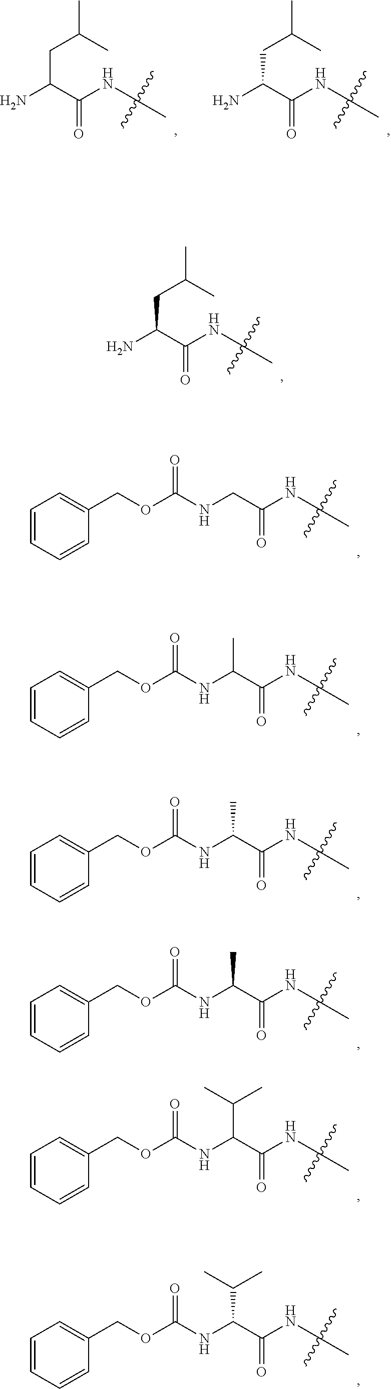

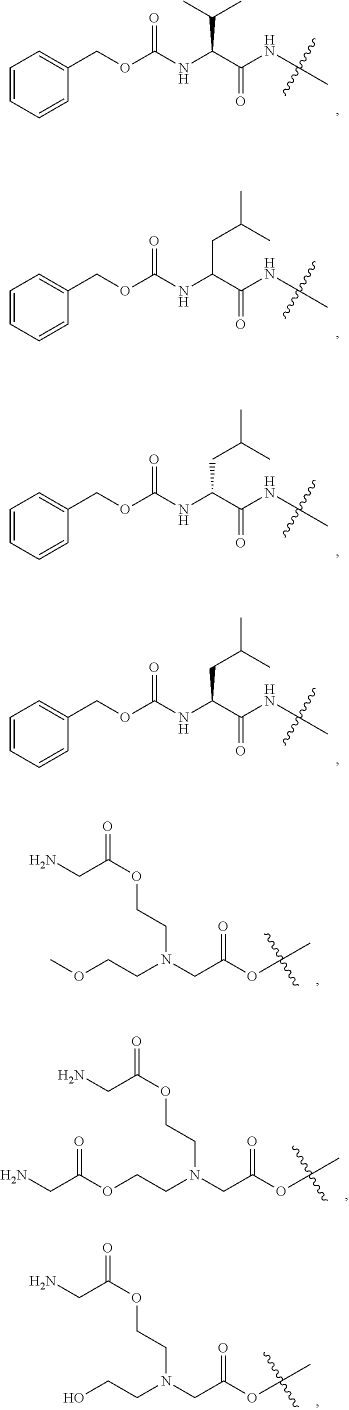

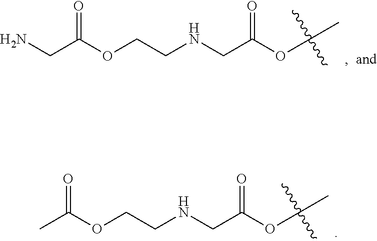 [SMILES: *OC(=O)CNCCOC(=O)CN ; *OC(=O)CNCCOC(C)=O]

The compound may be a compound of Formula (I-II) wherein R^{7A} and R^{7B} are each hydrogen, R^{7C} and R^{7D} are each bonded to the same oxygen atom to form an oxo group, and R^{7E} and R^{7F} are each alkyl (e.g., methyl).

The compound may be a compound of Formula (I-II) wherein Y¹ is —NR¹—, —O—, R^{7A} and R^{7B} are each hydrogen, R^{7C} and R^{7D} are each bonded to the same oxygen atom to form an oxo group, R^{7E} and R^{7F} are each alkyl (e.g., methyl), and Y² is heteroalkyl or —OR³. For example, the compound may be a compound of Formula (I-II) wherein Y¹ is —O—, R^{7A} and R^{7B} are each hydrogen, R^{7C} and R^{7D} are each bonded to the same oxygen atom to form an oxo group, R^{7E} and R^{7F} are each alkyl (e.g., methyl), and Y² is —OH. Alternatively, the compound may be a compound of Formula (I-II) wherein Y¹ is —O—, R^{7A} and R^{7B} are each hydrogen, R^{7C} and R^{7D} are each bonded to the same oxygen atom to form an oxo group, R^{7E} and R^{7F} are each alkyl (e.g., methyl), and Y² is

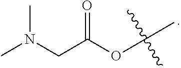 [SMILES: *OC(=O)CN(C)C]

Alternatively, the compound may be a compound of Formula (I-II) wherein Y¹ is —O—, R^{7A} and R^{7B} are each hydrogen, R^{7C} and R^{7D} are each bonded to the same oxygen atom to form an oxo group, R^{7E} and R^{7F} are each alkyl (e.g., methyl), and Y² is

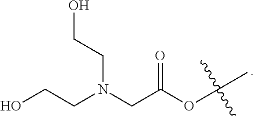 [SMILES: *OC(=O)CN(CCO)CCO]

Alternatively, the compound may be a compound of Formula (I-II) wherein Y¹ is —O—, R^{7A} and R^{7B} are each hydrogen, R^{7C} and R^{7D} are each bonded to the same oxygen atom to form an oxo group, R^{7E} and R^{7F} are each alkyl (e.g., methyl), and Y² is

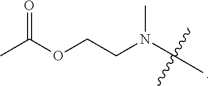 [SMILES: *N(C)CCOC(C)=O]

Alternatively, the compound may be a compound of Formula (I-II) wherein Y¹ is —O—, R^{7A} and R^{7B} are each hydrogen, R^{7C} and R^{7D} are each bonded to the same oxygen atom to form an oxo group, R^{7E} and R^{7F} are each alkyl (e.g., methyl), and Y² is

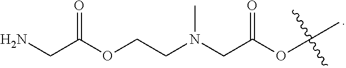 [SMILES: *OC(=O)CN(C)CCOC(=O)CN]

Alternatively, the compound may be a compound of Formula (I-II) wherein Y¹ is —O—, R^{7A} and R^{7B} are each hydrogen, R^{7C} and R^{7D} are each bonded to the same oxygen atom to form an oxo group, R^{7E} and R^{7F} are each alkyl (e.g., methyl), and Y² is

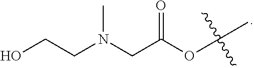 [SMILES: *OC(=O)CN(C)CCO]

A compound of the present disclosure may be a compound of Formula (IA)

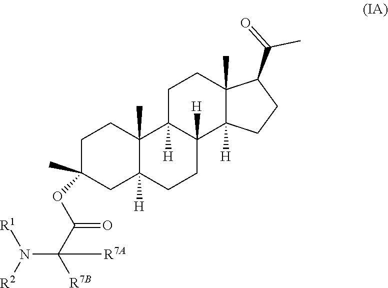

or a pharmaceutically acceptable salt, solvate, hydrate, tautomer, or stereoisomer thereof, wherein
R¹ and R² are each independently hydrogen, alkyl, alkenyl, alkynyl, heteroalkyl, cycloalkyl, heterocyclyl, aryl, heteroaryl, alkylene-cycloalkyl, alkylene-heterocyclyl, alkylene-aryl, alkylene-heteroaryl, heteroalkylene-cycloalkyl, heteroalkylene-heterocyclyl, heteroalkylene-aryl, heteroalkylene-heteroaryl, cycloalkylene-alkyl, cycloalkylene-heteroalkyl, heterocyclylene-alkyl, or heterocyclylene-heteroalkyl, wherein the alkyl, alkylene, alkenyl, alkynyl, heteroalkyl, heteroalkylene, cycloalkyl, cycloalkylene, heterocyclyl, heterocyclylene, aryl, or heteroaryl is optionally substituted;
or R¹ and R², together with the atoms to which they are attached, form a 3-7 membered cycloalkyl, heterocyclyl, aryl, or heteroaryl, wherein the cycloalkyl, heterocyclyl, aryl, or heteroaryl is optionally substituted; and
R^{7A} and R^{7B} are each independently hydrogen or optionally substituted alkyl.

The compound may be a compound of Formula (I), (I-I), or (IA), wherein the X group of Formula (I) or (I-I), or the —N(R¹)(R²) group of Formula (IA), is a moiety selected from the group consisting of

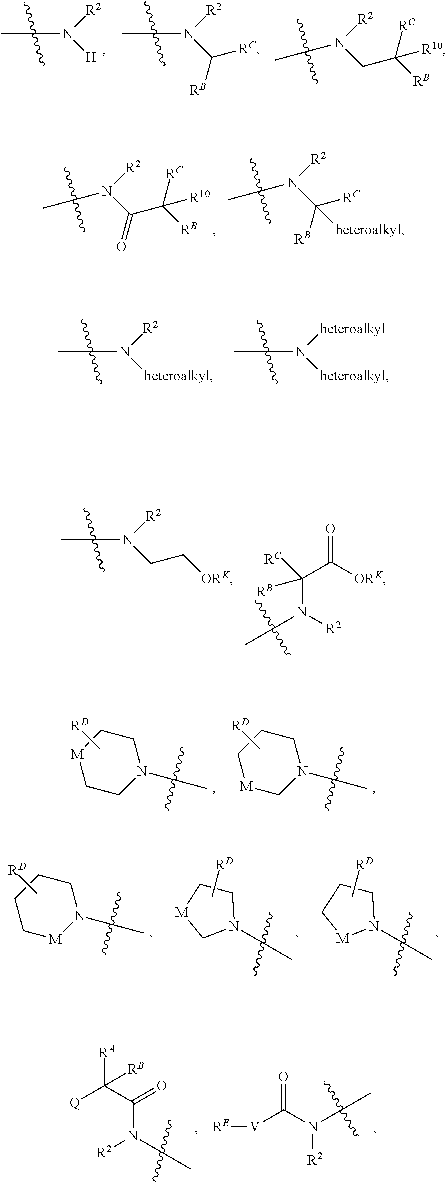

wherein
R² is hydrogen or alkyl;
M is-C(H)(R^{D})— (e.g., —CH₂—), —O—, or —N(R^{D})— (e.g., —N(Me)—);
Q is heteroalkyl or heterocyclyl, wherein the heteroalkyl or heterocyclyl is optionally substituted;
V is —O—, —N(H)—, or —N(Me)—;
R^{A}, R^{B}, R^{C}, R^{D}, R^{E} and R^{K} are each independently hydrogen, alkyl, heteroalkyl, cycloalkyl, heterocyclyl, aryl, or heteroaryl, wherein the alkyl, heteroalkyl, cycloalkyl, heterocyclyl, aryl, or heteroaryl is optionally substituted; and
[symbol] denotes the point of attachment of X to Z, or X to Y when Z is absent, in Formula (I); the point of attachment of X to the carbon atom of the —C(R^{7A})(R^{7B}) group in Formula (I-I); or the point of attachment of the nitrogen atom to the carbon atom of the —C(R^{7A})(R^{7B}) group in Formula (IA).

In some embodiments, the X group of Formula (I) or Formula (I-I), or the —N(R¹)(R²) group of Formula (IA), is a moiety selected from the group consisting of

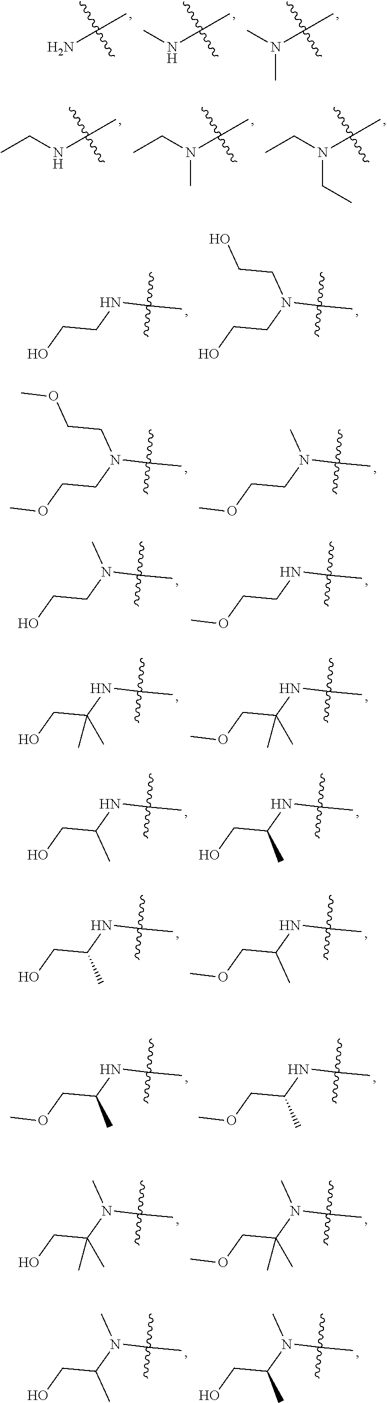

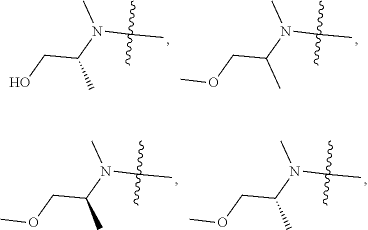

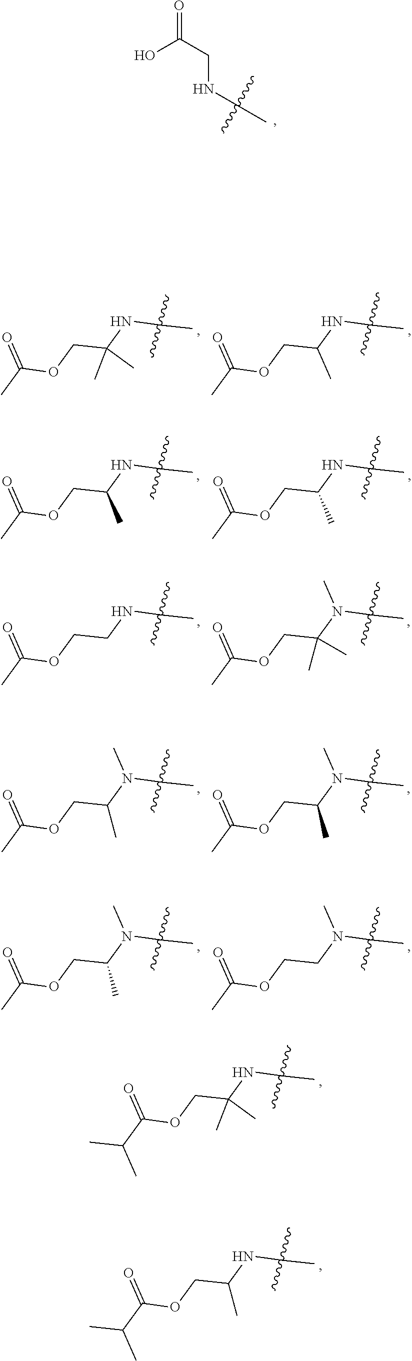

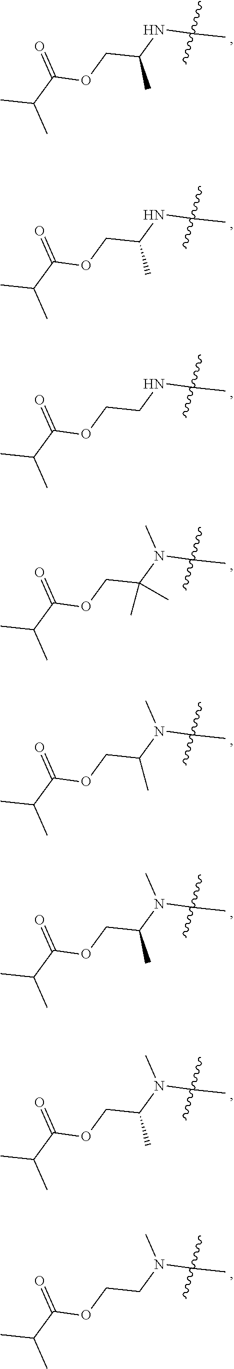

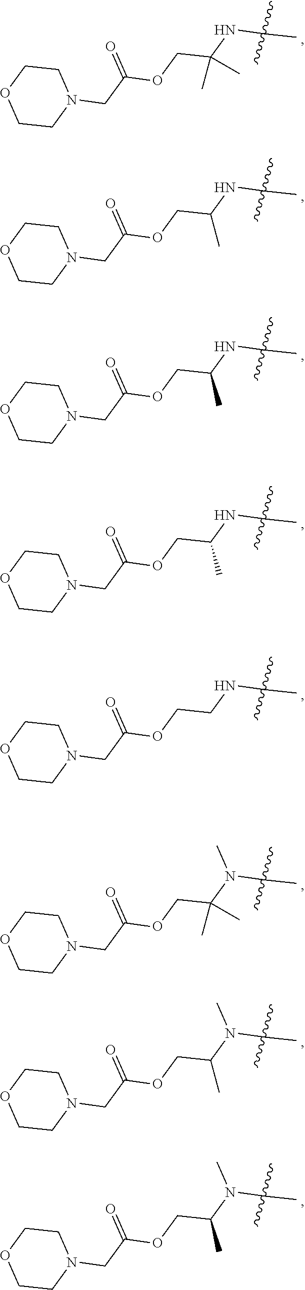

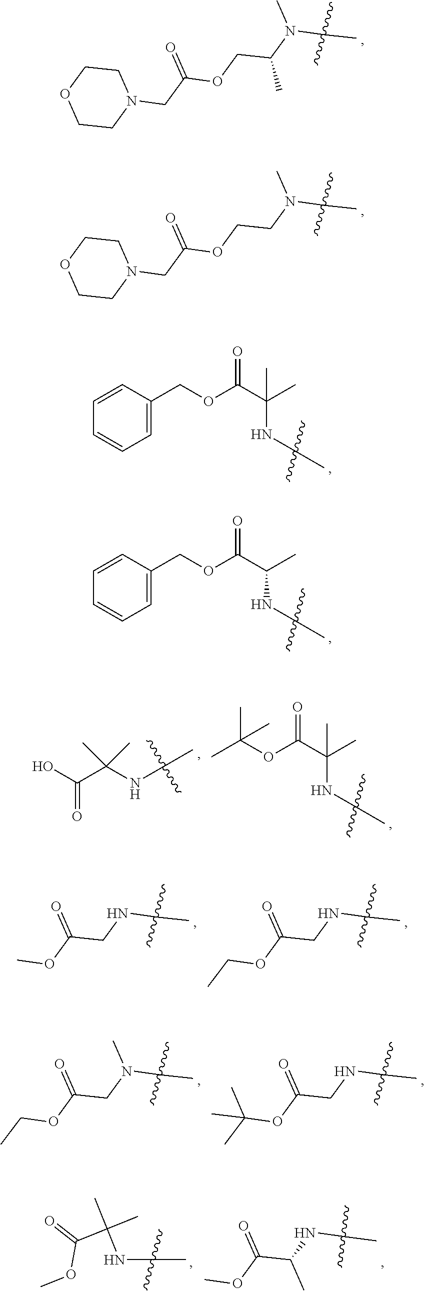

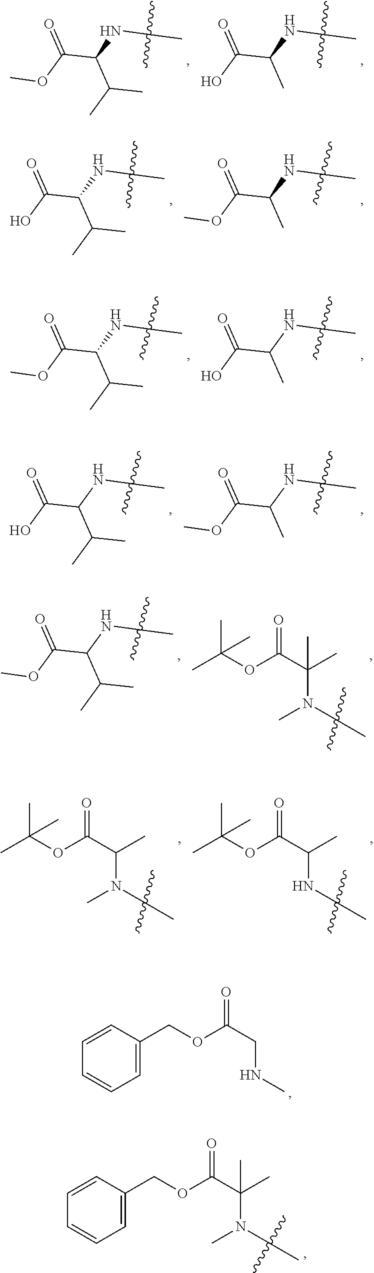

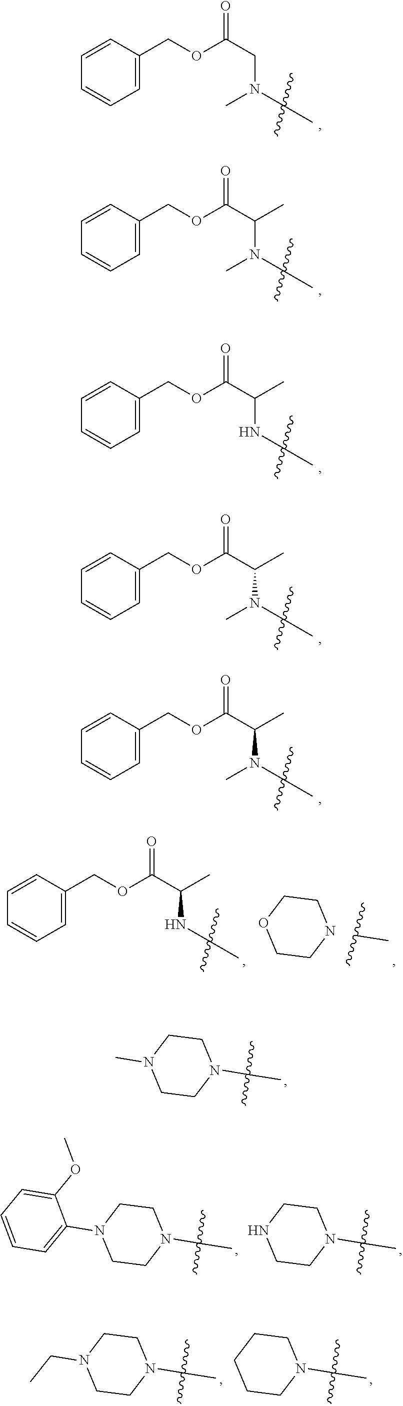

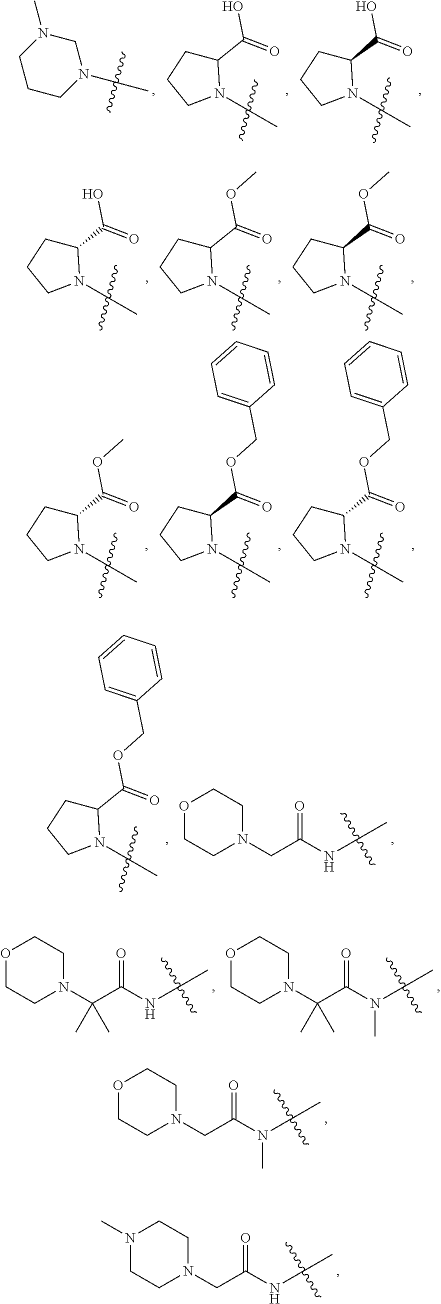

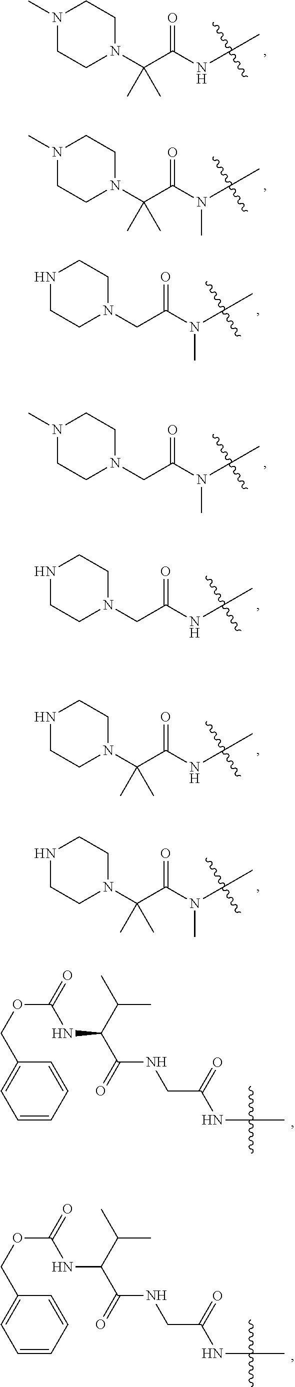

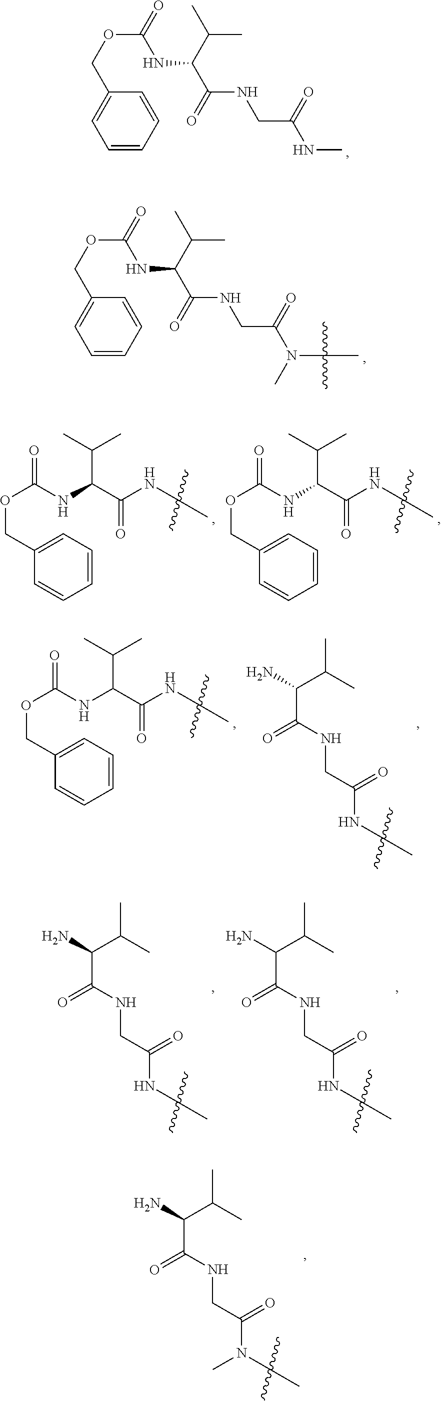

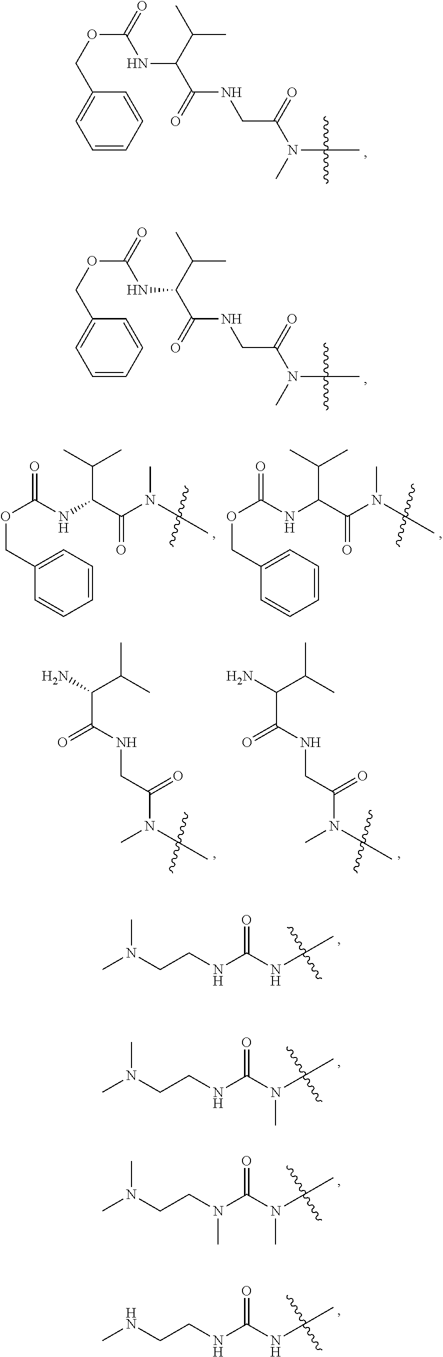

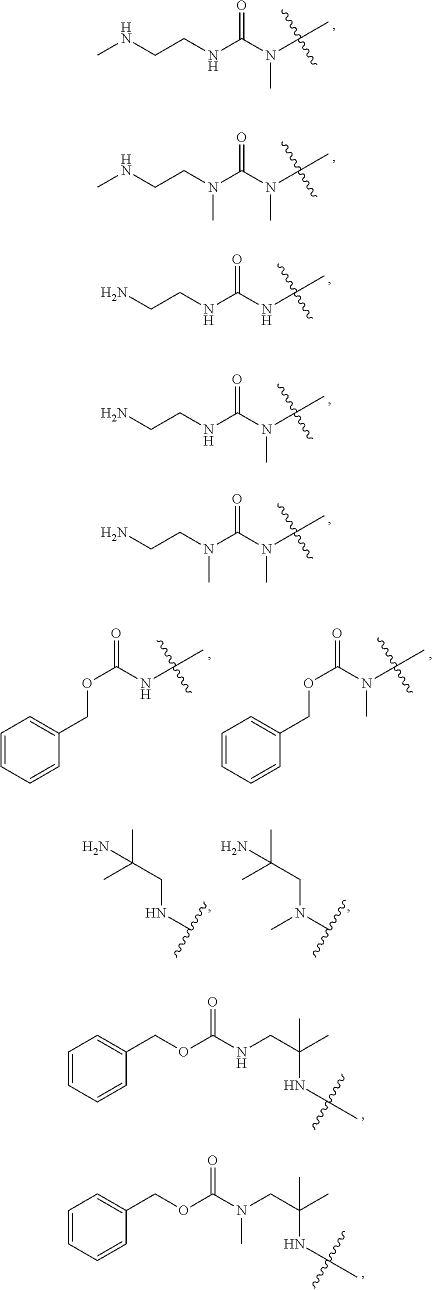

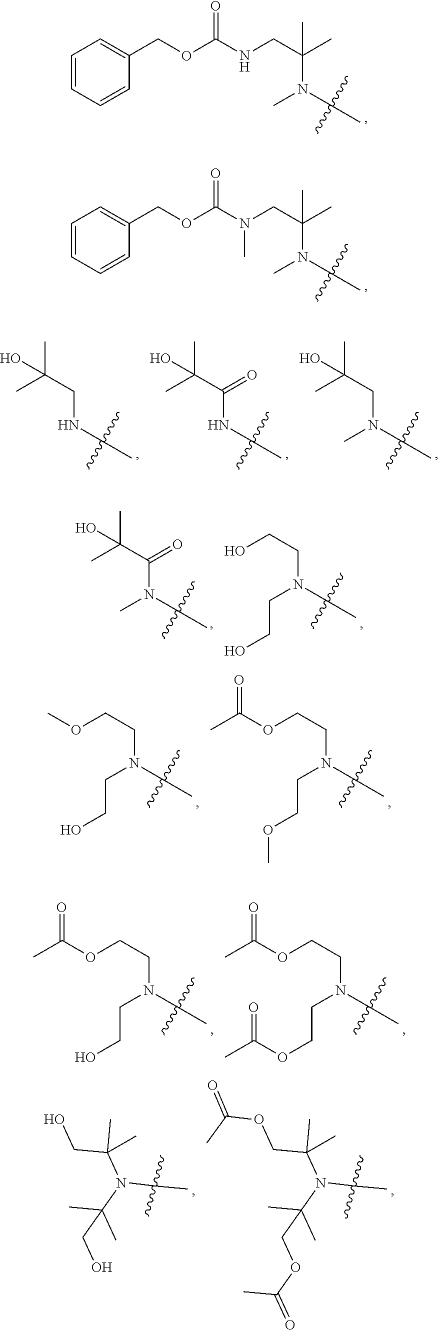

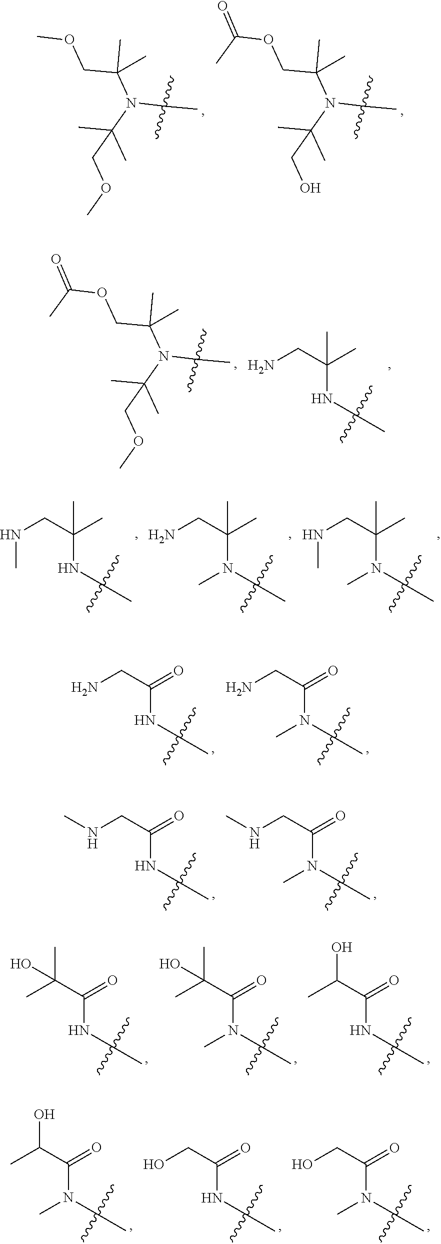

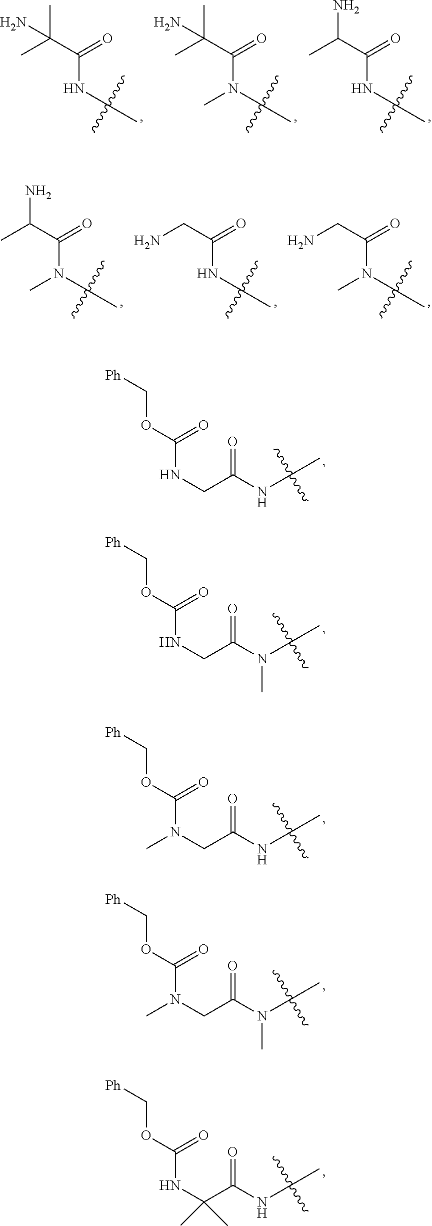

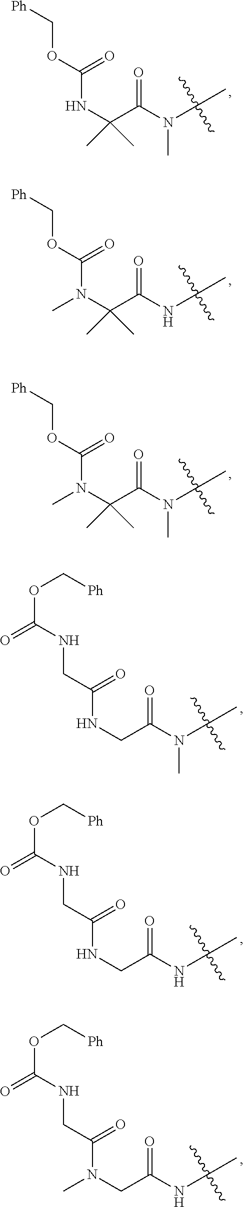

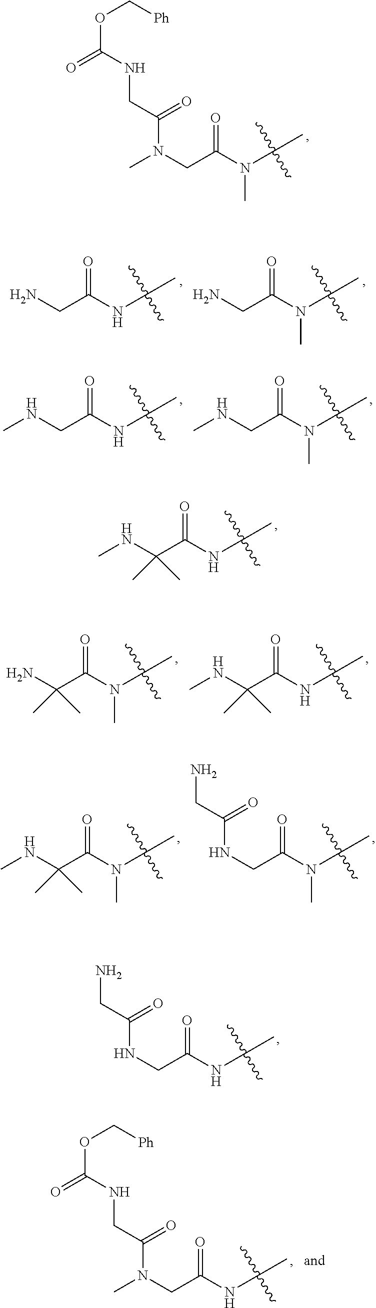

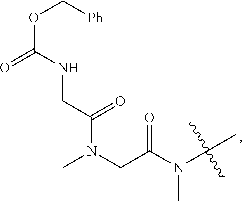

wherein [symbol] denotes the point of attachment of X to Z, or X to Y when Z is absent, in Formula (I); the point of attachment of X to the carbon atom of the —C(R^{7A})(R^{7B}) group in Formula (I-I); or the point of attachment of the nitrogen atom to the carbon atom of the —C(R^{7A})(R^{7B}) group in Formula (IA).

A compound of the present disclosure may be a compound of Formula (IA-a)

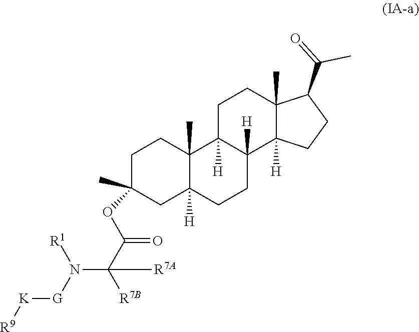

or a pharmaceutically acceptable salt, solvate, hydrate, tautomer, or stereoisomer thereof, wherein
G is absent, —C(O)—, or —C(R^{8A})(R^{8B})— (e.g., —CH₂—),
K is alkylene, heteroalkylene, —C(O)—, —OC(O)—, —C(O)O—, —O—, —O—(CH₂)—, or —(CH₂)—O—, wherein the alkylene or heteroalkylene is optionally substituted;
R², R^{7A}, R^{7B}, R^{8A}, and R^{8B} are each independently hydrogen or optionally substituted alkyl; and
R⁹ is hydrogen, alkyl, alkenyl, alkynyl, heteroalkyl, cycloalkyl, heterocyclyl, aryl, or heteroaryl, wherein the alkyl, alkenyl, alkynyl, heteroalkyl, cycloalkyl, heterocyclyl, aryl, or heteroaryl is optionally substituted.

In some embodiments, the compound of Formula (I), (I-I), or (IA) is a compound of Formula (IA-a), wherein G is —C(R^{8A})(R^{8B})—; K is —OC(O)—; R², R^{7A}, R^{7B}, and R⁹ are each hydrogen; and R^{8A} and R^{8B} are each independently hydrogen or alkyl (e.g., methyl), wherein the carbon atom of the carbonyl group in —OC(O)— is bonded to G.

A compound of the present disclosure may be a compound of Formula (IA-b)

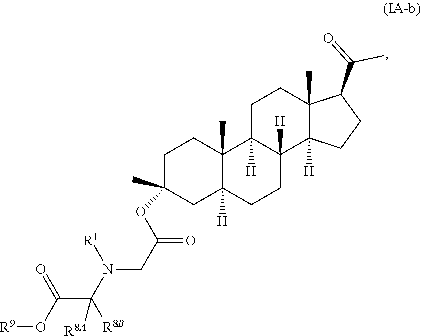

or a pharmaceutically acceptable salt, solvate, hydrate, tautomer, or stereoisomer thereof, wherein
R², R^{8A}, and R^{8B} are each independently hydrogen or optionally substituted alkyl; and
R⁹ is hydrogen, alkyl, alkenyl, alkynyl, heteroalkyl, cycloalkyl, heterocyclyl, aryl, or heteroaryl, wherein the alkyl, alkenyl, alkynyl, heteroalkyl, cycloalkyl, heterocyclyl, aryl, or heteroaryl is optionally substituted.

For example, the compound of Formula (I), (I-I), (IA), or (IA-a) may be a compound of Formula (IA-b), wherein R² is hydrogen; and R^{8A} and R^{8B} are each independently methyl.

A compound of the present disclosure may be a compound of Formula (IB):

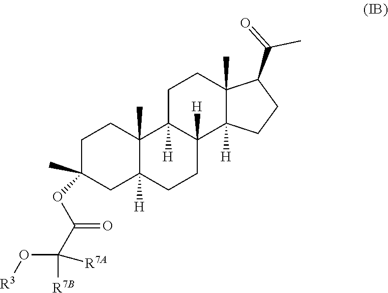

or a pharmaceutically acceptable salt, solvate, hydrate, tautomer, or stereoisomer thereof, wherein
R³ is hydrogen, alkyl, alkenyl, alkynyl, heteroalkyl, cycloalkyl, heterocyclyl, aryl, heteroaryl, alkylene-cycloalkyl, alkylene-heterocyclyl, alkylene-aryl, alkylene-heteroaryl, heteroalkylene-cycloalkyl, heteroalkylene-heterocyclyl, heteroalkylene-aryl, heteroalkylene-heteroaryl, cycloalkylene-alkyl, cycloalkylene-heteroalkyl, heterocyclylene-alkyl, or heterocyclylene-heteroalkyl, wherein the alkyl, alkylene, alkenyl, alkynyl, heteroalkyl, heteroalkylene, cycloalkyl, cycloalkylene, heterocyclyl, heterocyclylene, aryl, or heteroaryl is optionally substituted; and
R^{7A} and R^{7B} are each independently hydrogen or optionally substituted alkyl.

The X of Formula (I) or (II), or the —O—(R³) group of Formula (IB), may be a moiety selected from the group consisting of

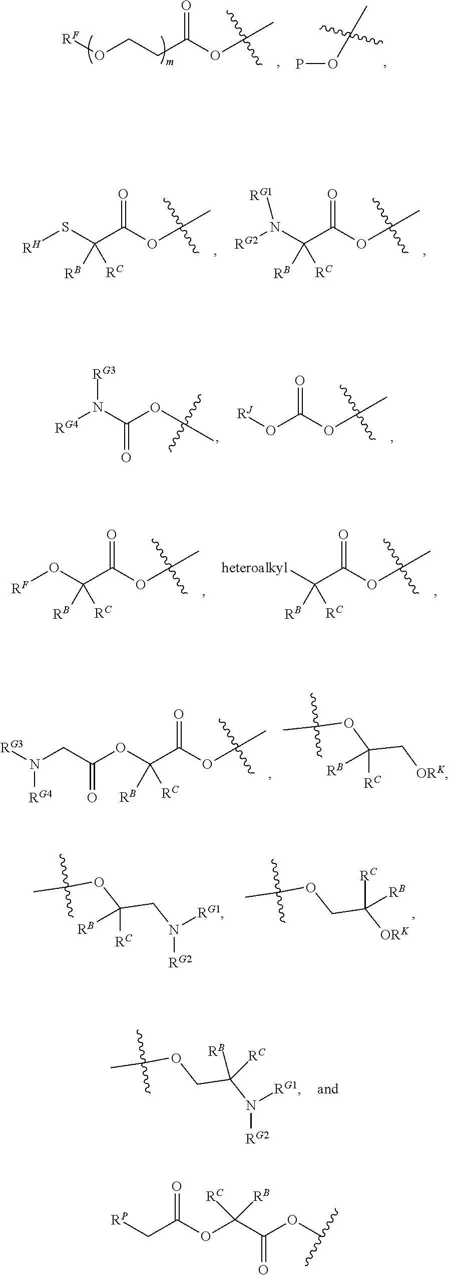

wherein
P is an amino acid residue (e.g., glycine, alanine, valine, leucine, or proline), that may be a D-amino acid residue or L-amino acid residue, and is optionally substituted (e.g., at a terminal nitrogen atom, e.g., by a carbonate group, e.g., a carboxybenzyl (Cbz) group),
or P is a dipeptide moiety (e.g., a leucine-leucine moiety) that may comprise D-amino acids, L-amino acids, or a combination thereof, and is optionally substituted (e.g., at one or more nitrogen atoms, e.g., at a terminal nitrogen atom, e.g., by a carbonate group, e.g., a carboxybenzyl (Cbz) group);
R^{B} and R^{C} are each independently hydrogen, alkyl, heteroalkyl, cycloalkyl, heterocyclyl, aryl, or heteroaryl, wherein the alkyl, heteroalkyl, cycloalkyl, heterocyclyl, aryl, or heteroaryl is optionally substituted;
R^{F} and R^{H} are each independently hydrogen or alkyl;
R^{G1} and R^{G2} are each independently hydrogen, alkyl, or an amino acid residue (e.g., glycine, alanine, valine, leucine or proline), that may be a D-amino acid residue or L-amino acid residue, and is optionally substituted (e.g., at a terminal nitrogen atom, e.g., by a carbonate group, e.g., a carboxybenzyl (Cbz) group),
or R^{G1} and R^{G2}, together with the atoms to which they are attached, form a 3-7 membered cycloalkyl, heterocyclyl, aryl, or heteroaryl, wherein the cycloalkyl, heterocyclyl, aryl, or heteroaryl is optionally substituted;
R^{G3} and R^{G4} are each independently hydrogen, alkyl, or heteroalkyl, wherein the alkyl or heteroalkyl is optionally substituted;
or R^{G3} and R^{G4}, together with the atoms to which they are attached, form a 3-7 membered cycloalkyl, heterocyclyl, aryl, or heteroaryl, wherein the cycloalkyl, heterocyclyl, aryl, or heteroaryl is optionally substituted;
R^{J} is hydrogen, alkyl, alkylene-heterocyclyl, heteroalkyl, or heterocyclyl, wherein the alkyl, alkylene, heteroalkyl, or heterocyclyl is optionally substituted;
R^{K} is hydrogen, alkyl, heteroalkyl, cycloalkyl, heterocyclyl, aryl, or heteroaryl, wherein the alkyl, heteroalkyl, cycloalkyl, heterocyclyl, aryl, or heteroaryl is optionally substituted;
R^{P} is halo, —NH-alkyl, —NH-heteroalkyl, —N-(alkyl)₂ (e.g., —N(Me)₂), —N-(heteroalkyl)₂ (e.g., —N(CH₂CH₂OH)₂), or heterocyclyl (e.g., morpholinyl);
m is an integer of 1-5; and [symbol]
[symbol] denotes the point of attachment of X to Z, or X to Y when Z is absent, in Formula (I); [symbol] denotes the point of attachment of X to the carbon atom of the —C(R^{7A})(R^{7B}) group in Formula (I-I); and [symbol] denotes the point of attachment of the oxygen atom to the carbon atom of the —C(R^{7A})(R^{7B}) group in Formula (IB).

In some aspects, the compound is a compound of Formula (I), (I-I), or (IB), wherein the X group of Formula (I) or (I-I), or the —O—R³ group of Formula (IB), is a moiety selected from the group consisting of

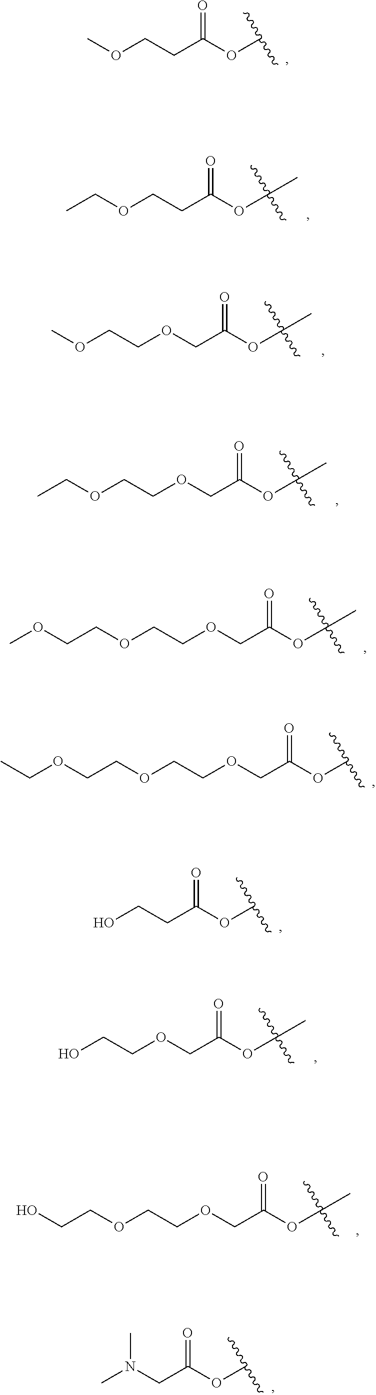

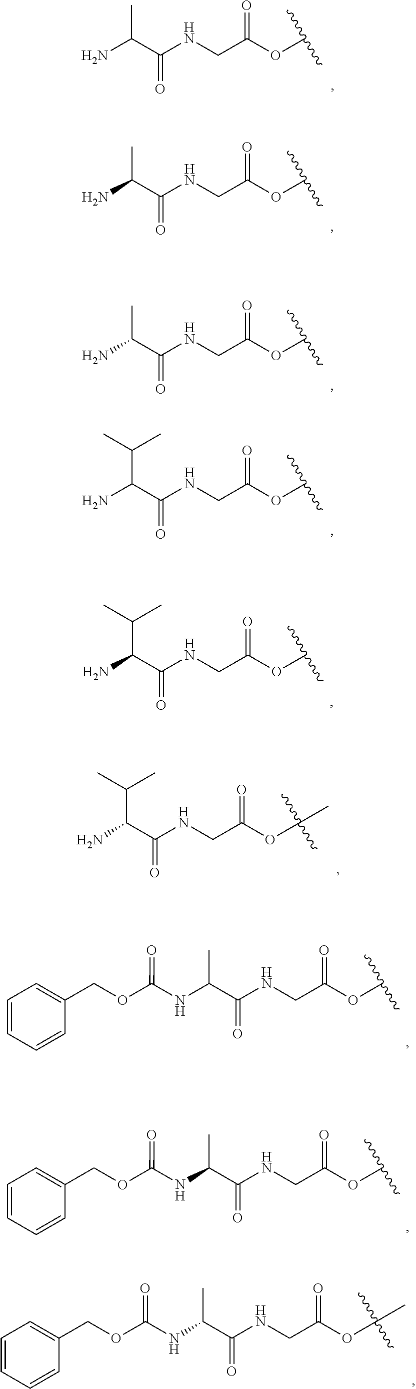

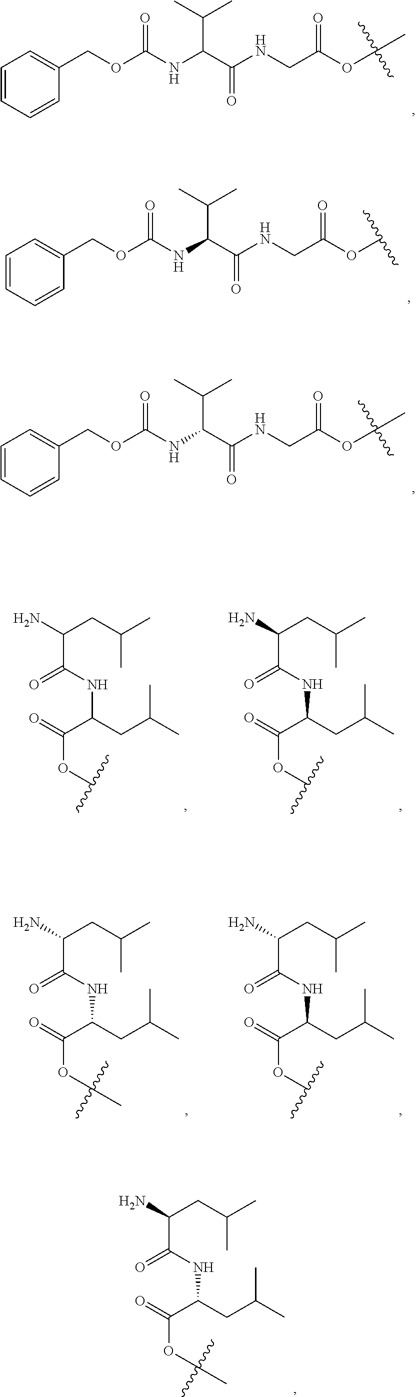

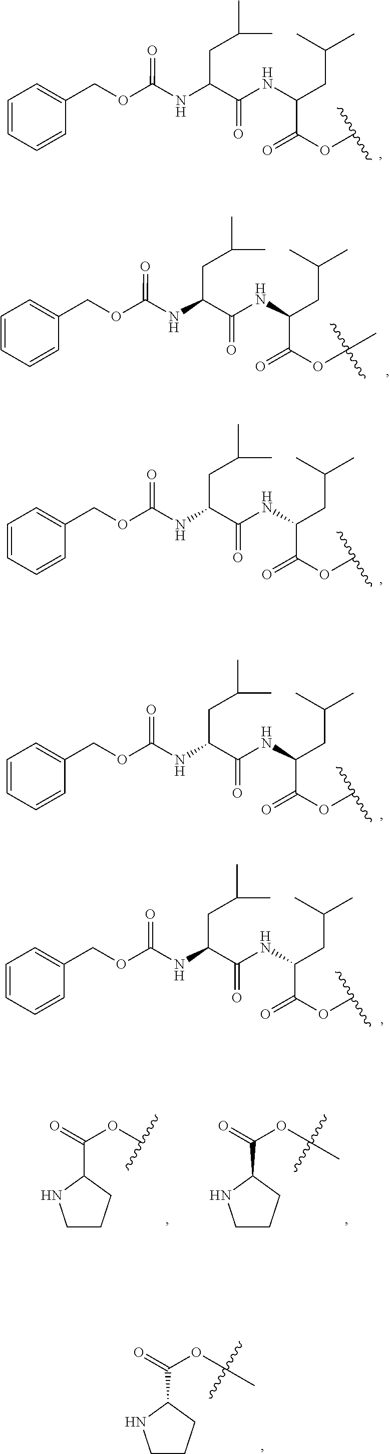

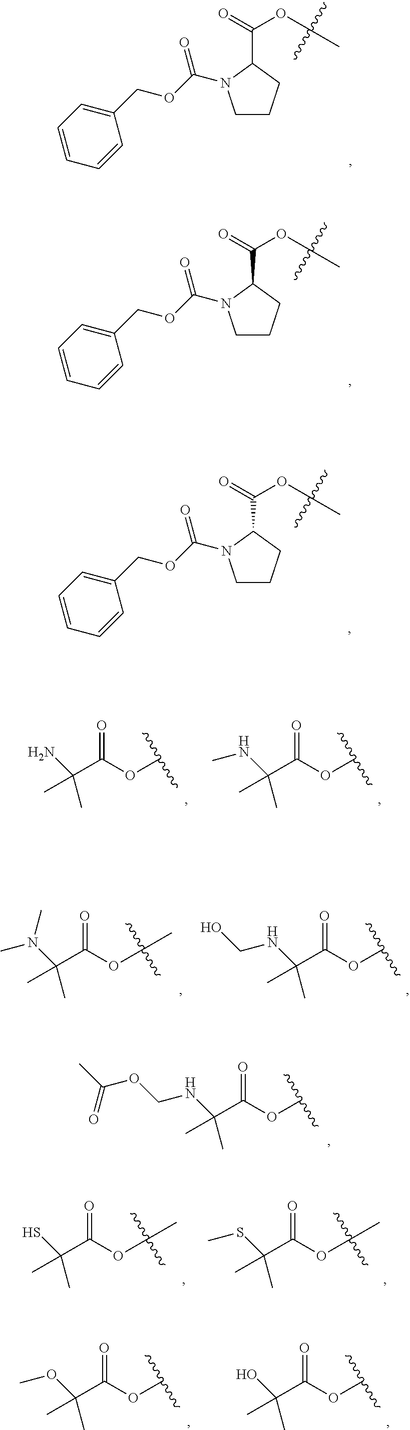

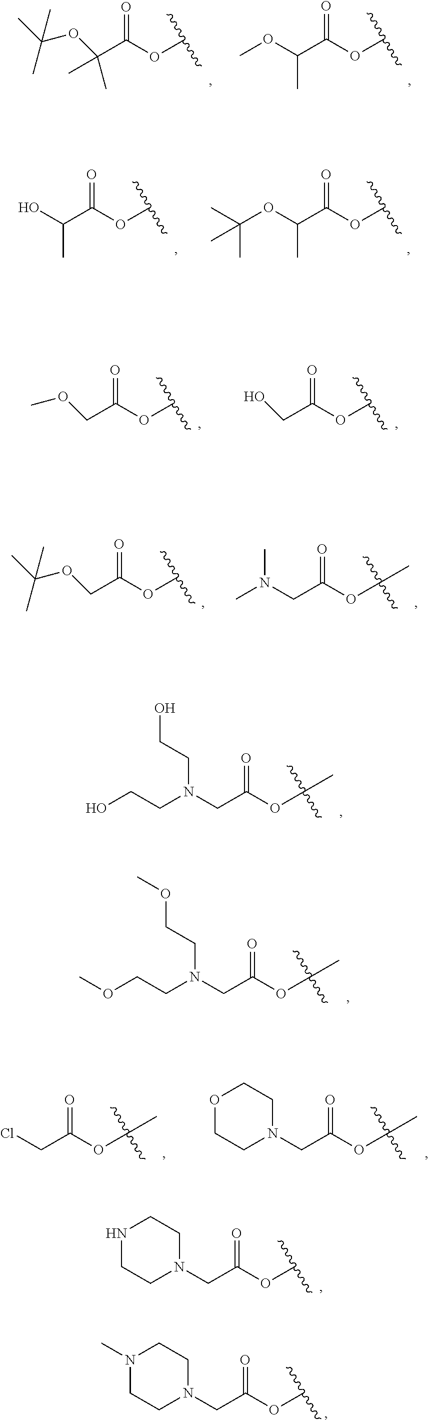

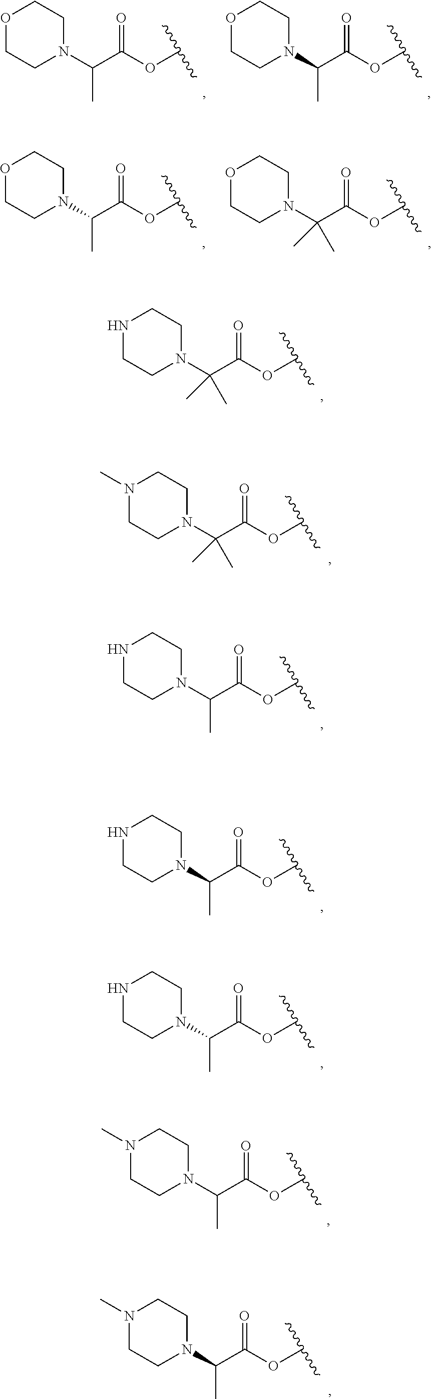

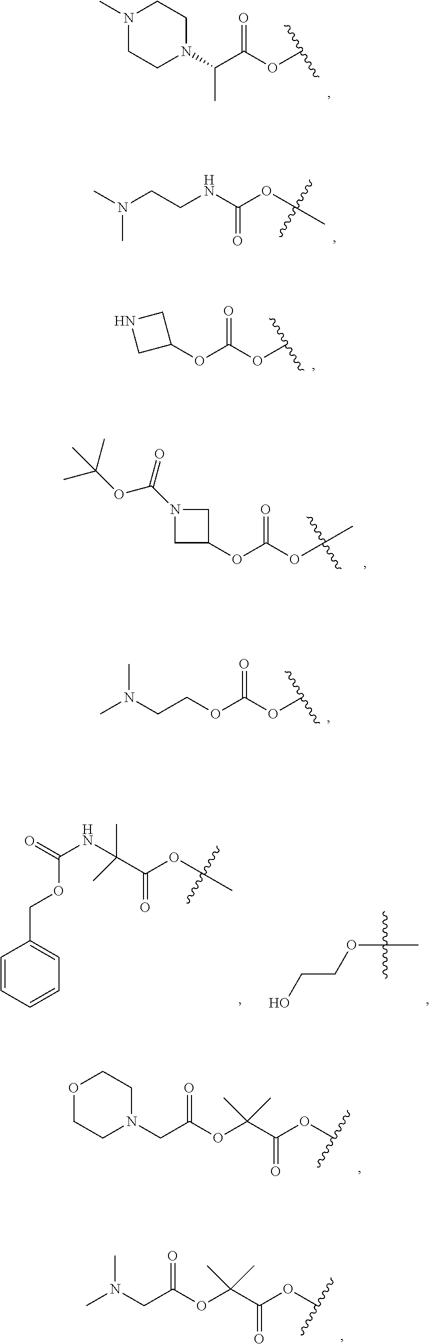

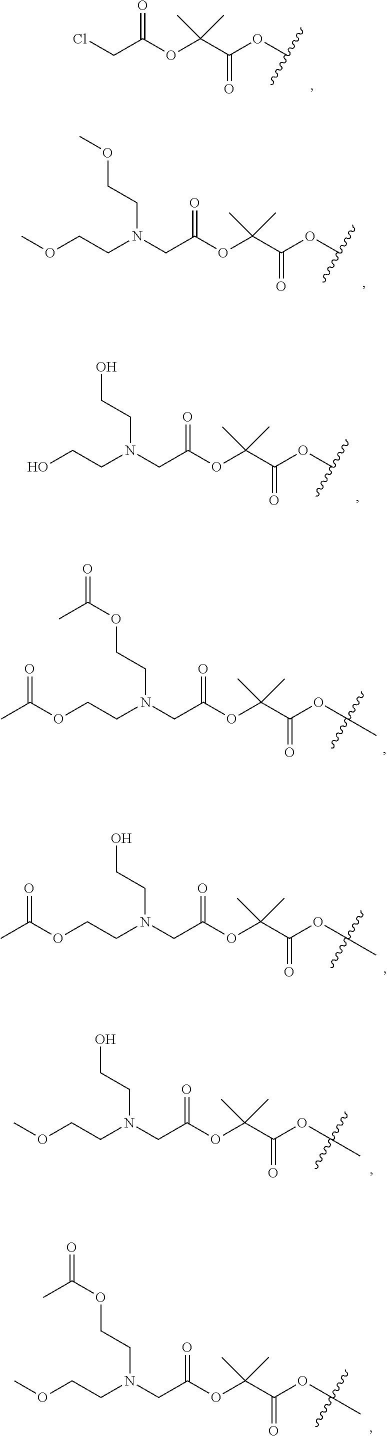

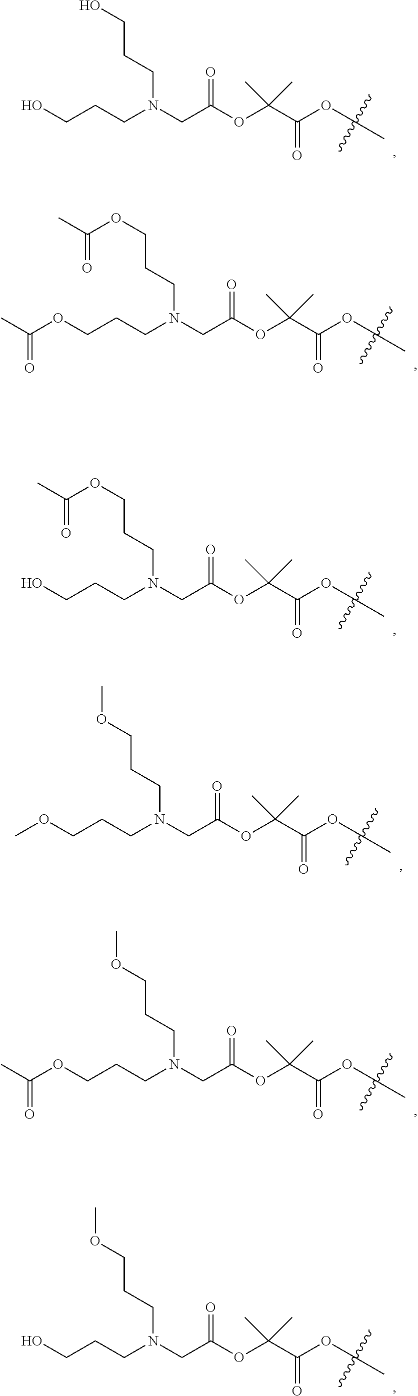

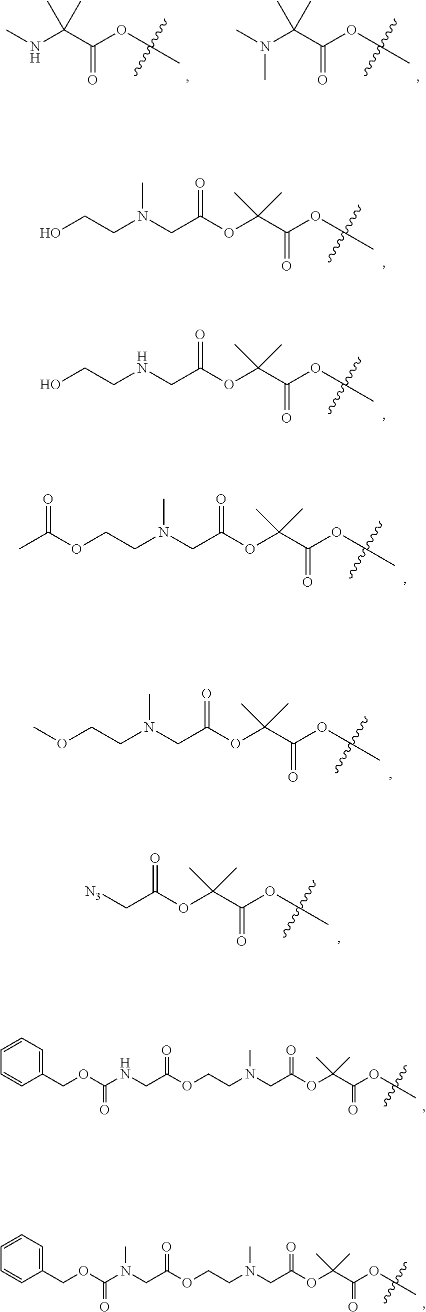

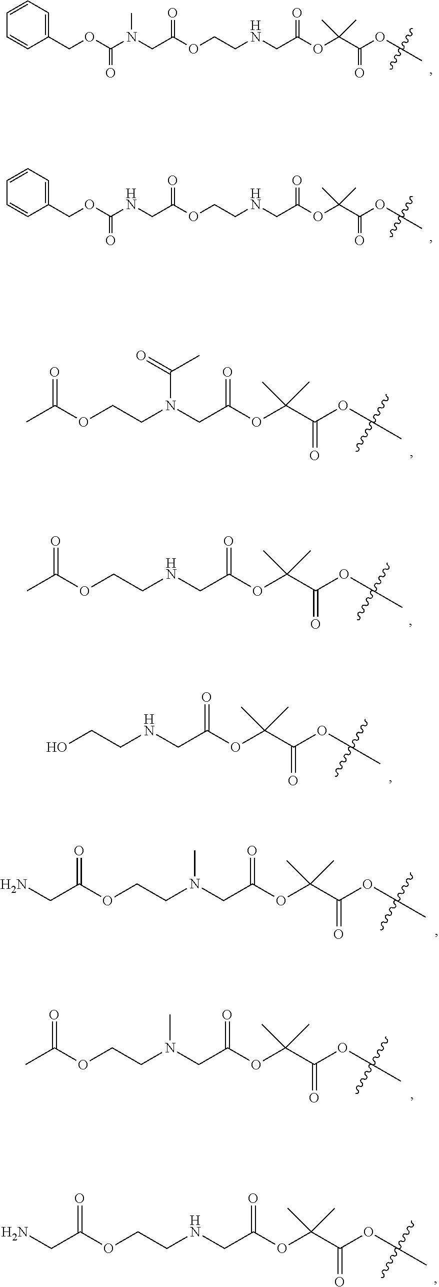

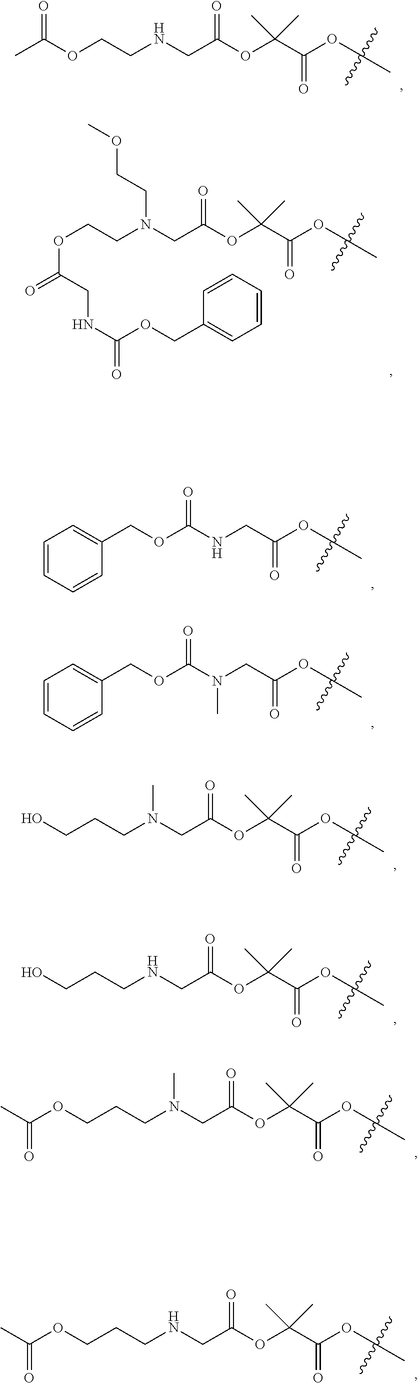

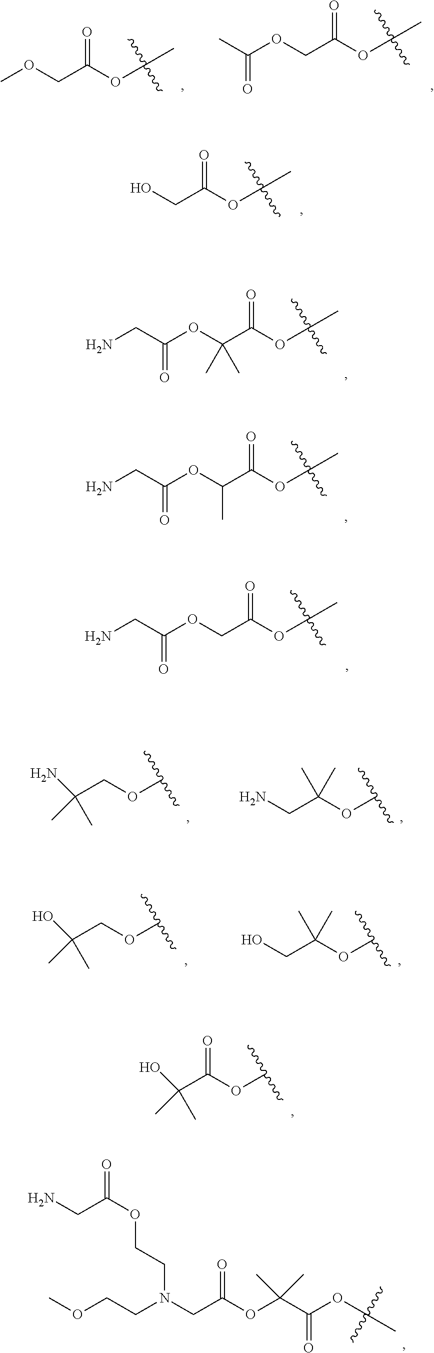

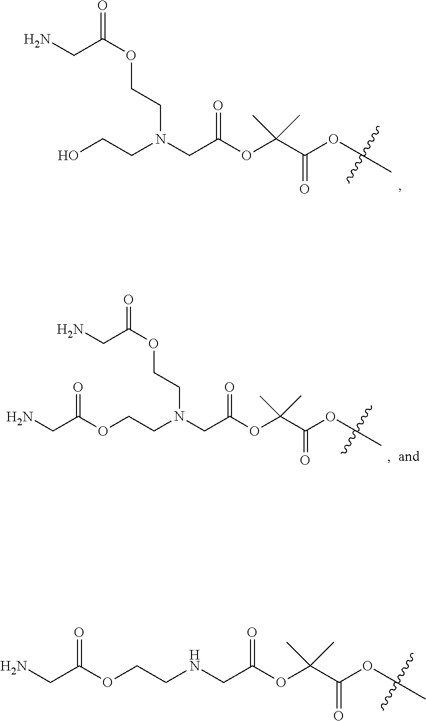

Wherein [symbol] denotes the point of attachment of X to Z, or X to Y when Z is absent, in Formula (I); the point of attachment of X to the carbon atom of the —C(R^{7A})(R^{7B}) group in Formula (I-I); or the point of attachment of the oxygen atom to the carbon atom of the —C(R^{7A})(R^{7B}) group in Formula (IB).

A compound of the present disclosure may be a compound of Formula (IC)

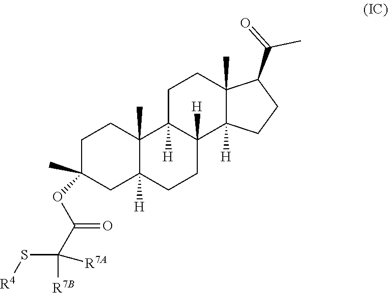

or a pharmaceutically acceptable salt, solvate, hydrate, tautomer, or stereoisomer thereof, wherein
R⁴ is hydrogen, alkyl, alkenyl, alkynyl, heteroalkyl, cycloalkyl, heterocyclyl, aryl, heteroaryl, alkylene-cycloalkyl, alkylene-heterocyclyl, alkylene-aryl, alkylene-heteroaryl, heteroalkylene-cycloalkyl, heteroalkylene-heterocyclyl, heteroalkylene-aryl, or heteroalkylene-heteroaryl, wherein the alkyl, alkylene, alkenyl, alkynyl, heteroalkyl, heteroalkylene, cycloalkyl, cycloalkylene, heterocyclyl, heterocyclylene, aryl, or heteroaryl is optionally substituted; and
R^{7A} and R^{7B} are each independently hydrogen or optionally substituted alkyl.

In some embodiments, the X group of Formula (I) or (I-I), or the —S—(R⁴) group of Formula (IC), is a moiety selected from the group consisting of

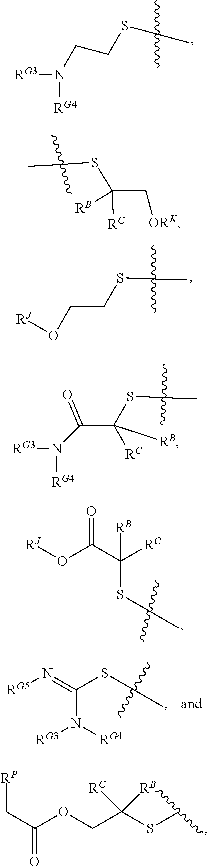

wherein
R^{B} and R^{C} are each independently hydrogen, alkyl, heteroalkyl, cycloalkyl, heterocyclyl, aryl, or heteroaryl, wherein the alkyl, heteroalkyl, cycloalkyl, heterocyclyl, aryl, or heteroaryl is optionally substituted;
R^{G3} and R^{G4} are each independently hydrogen, alkyl, or heteroalkyl, wherein the alkyl or heteroalkyl is optionally substituted;
or R^{G3} and R^{G4}, together with the atoms to which they are attached, form a 3-7 membered cycloalkyl, heterocyclyl, aryl, or heteroaryl, wherein the cycloalkyl, heterocyclyl, aryl, or heteroaryl is optionally substituted;
R^{G5} is hydrogen, alkyl, or heteroalkyl, wherein the alkyl or heteroalkyl is optionally substituted;
R^{J} is hydrogen, alkyl, alkylene-heterocyclyl, heteroalkyl, or heterocyclyl, wherein the alkyl, alkylene, heteroalkyl, or heterocyclyl is optionally substituted;
R^{K} is hydrogen, alkyl, heteroalkyl, cycloalkyl, heterocyclyl, aryl, or heteroaryl, wherein the alkyl, heteroalkyl, cycloalkyl, heterocyclyl, aryl, or heteroaryl is optionally substituted;
R^{P} is halo, —NH-alkyl, —NH-heteroalkyl, —N-(alkyl)₂ (e.g., —N(Me)₂), —N-(heteroalkyl)₂ (e.g., —N(CH₂CH₂OH)₂), or heterocyclyl (e.g., morpholinyl); and
[symbol] denotes the point of attachment of X to Z, or X to Y when Z is absent, in Formula (I); the point of attachment of X to the carbon atom of the —C(R^{7A})(R^{7B}) group in Formula (I-I); or the point of attachment of the sulfur atom to the carbon atom of the —C(R^{7A})(R^{7B}) group in Formula (IC).

In some embodiments, the compound is a compound of Formula (I), (I-I), or (IC), wherein the X group of Formula (I) or (I-I), or the —S—R⁴ group of Formula (IC), is a moiety selected from the group consisting of

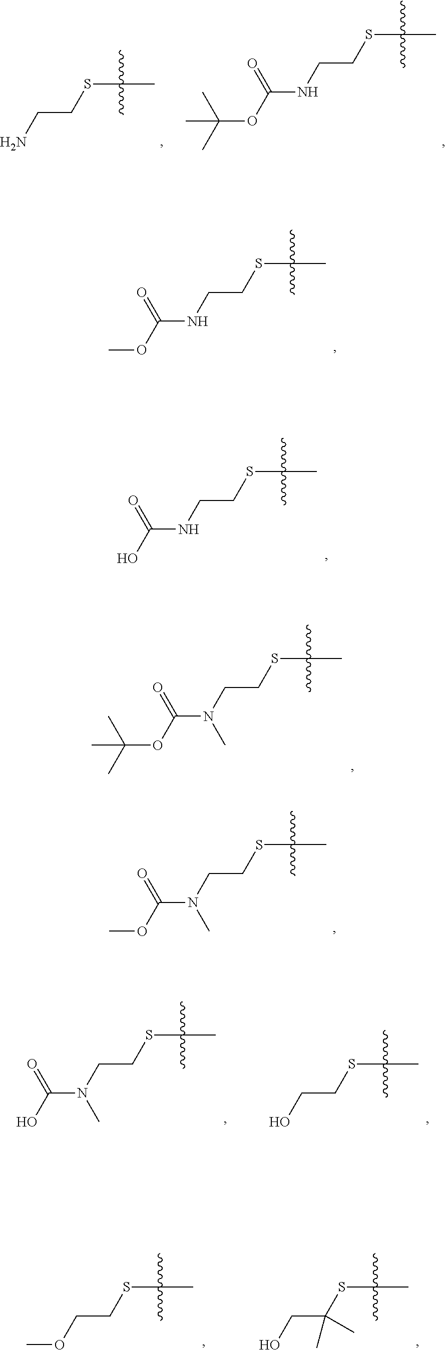

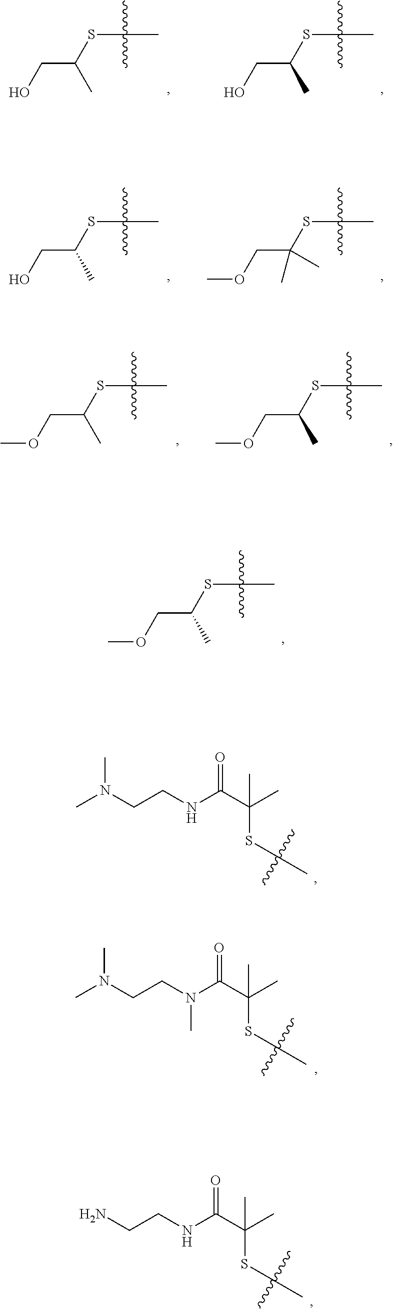

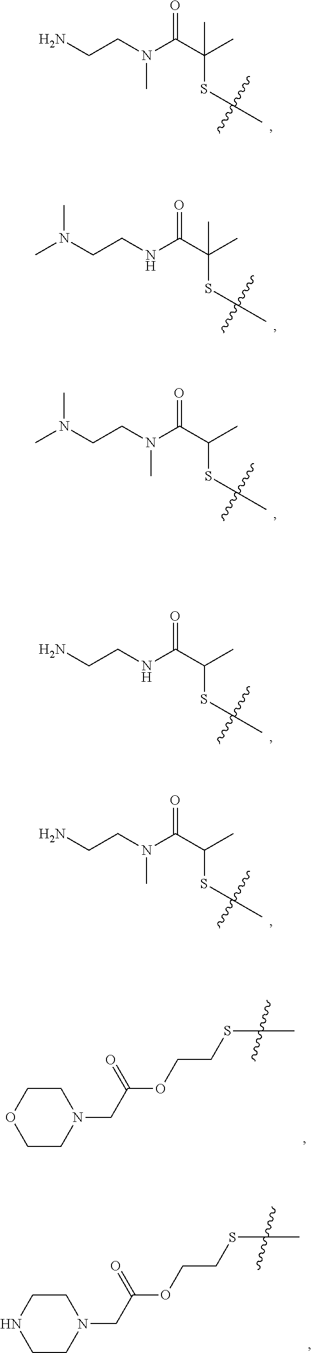

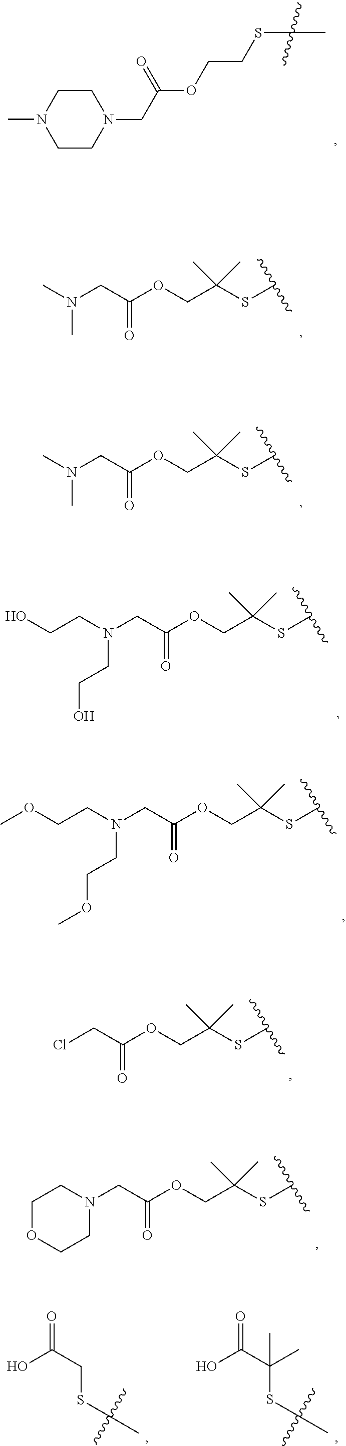

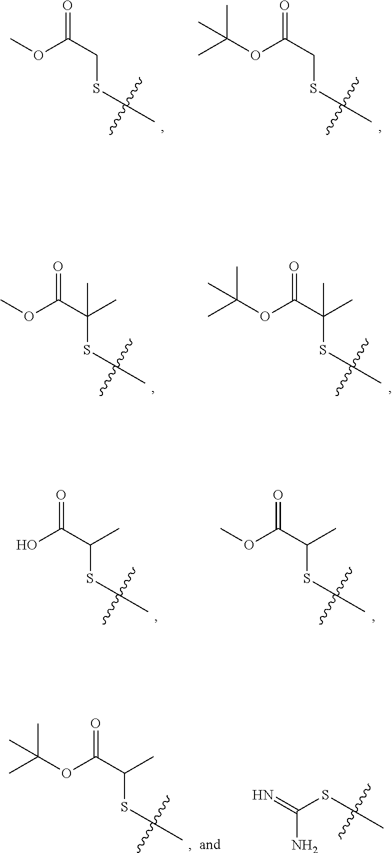

wherein [symbol] denotes the point of attachment of X to Z, or X to Y when Z is absent, in Formula (I); the point of attachment of X to the carbon atom of the —C(R^{7A})(R^{7B}) group in Formula (I-I); or the point of attachment of the sulfur atom to the carbon atom of the —C(R^{7A})(R^{7B}) group in Formula (IC).

A compound of the present disclosure may be a compound of Formula (ID)

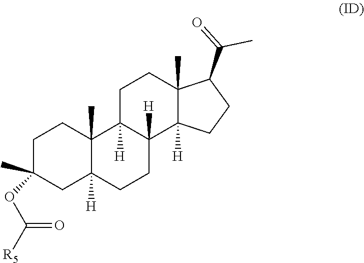

or a pharmaceutically acceptable salt, solvate, hydrate, tautomer, or stereoisomer thereof,
wherein
R⁵ is alkyl, alkenyl, alkynyl, heteroalkyl, haloalkyl, cycloalkyl, heterocyclyl, aryl, heteroaryl, haloalkyl, alkylene-heterocyclyl, alkylene-aryl, alkylene-heteroaryl, heteroalkylene-heterocyclyl, heteroalkylene-aryl, heteroalkylene-heteroaryl, cycloalkylene-alkyl, cycloalkylene-heteroalkyl, heterocyclylene-alkyl, or heterocyclylene-heteroalkyl, wherein the alkyl, alkylene, alkenyl, alkynyl, heteroalkyl, heteroalkylene, cycloalkyl, cycloalkylene, heterocyclyl, heterocyclylene, aryl, or heteroaryl is optionally substituted.

In some embodiments, the X group of Formula (I) or Formula (I-I), or the R⁵ group in Formula (ID), is a moiety selected from group consisting of

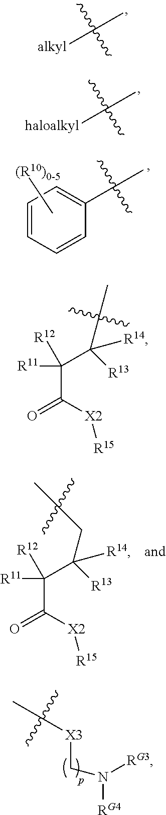

wherein
X2 and X3 are each independently —CH₂—, —O—, —S—, or —N(R^{G3})— (e.g., —NH—);
R¹⁰ is alkyl, alkenyl, alkynyl, haloalkyl, halo, —OR, —N(R^{G3})(R^{G4}); —NO₂, —CN, or —NC;
R¹¹, R¹², R¹³, and R¹⁴ are each independently hydrogen, alkyl, alkenyl, alkynyl, heteroalkyl, haloalkyl, halo, or —N(R^{G3})(R^{G4}), wherein the alkyl, alkenyl, alkynyl, heteroalkyl, or haloalkyl is optionally substituted,
or one of R¹¹ and R¹², and one of R¹³ and R¹⁴, together with the atoms to which they are attached, form a 3-7 membered cycloalkyl, heterocyclyl, aryl, or heteroaryl, wherein the cycloalkyl, heterocyclyl, aryl, or heteroaryl is optionally substituted;
R¹⁵ is hydrogen, alkyl, alkenyl, alkynyl, heteroalkyl, haloalkyl, or halo; wherein the alkyl, alkenyl, alkynyl, heteroalkyl, or haloalkyl is optionally substituted;
R^{G3} and R^{G4} are each independently hydrogen, alkyl, or heteroalkyl, wherein the alkyl or heteroalkyl is optionally substituted;
or R^{G3} and R^{G4}, together with the atoms to which they are attached, form a 3-7 membered cycloalkyl, heterocyclyl, aryl, or heteroaryl, wherein the cycloalkyl, heterocyclyl, aryl, or heteroaryl is optionally substituted;
R^{J} is hydrogen, alkyl, alkylene-heterocyclyl, heteroalkyl, or heterocyclyl, wherein the alkyl, alkylene, heteroalkyl, or heterocyclyl is optionally substituted;
and
p is an integer between 0 and 4; and
[symbol] denotes the point of attachment of X to Z, or X to Y when Z is absent, in Formula (I); the point of attachment of X to the carbon atom of the —C(R^{7A})(R^{7B}) group in Formula (I-I); or the point of attachment of the R⁵ group to the carbon atom of the —C(O)— group in Formula (ID).

The compound may be a compound of Formula (I), (I-I), or (ID), wherein the X group of Formula (I) or (I-I), or the R⁵ group of Formula (ID), is a moiety selected from the group consisting of

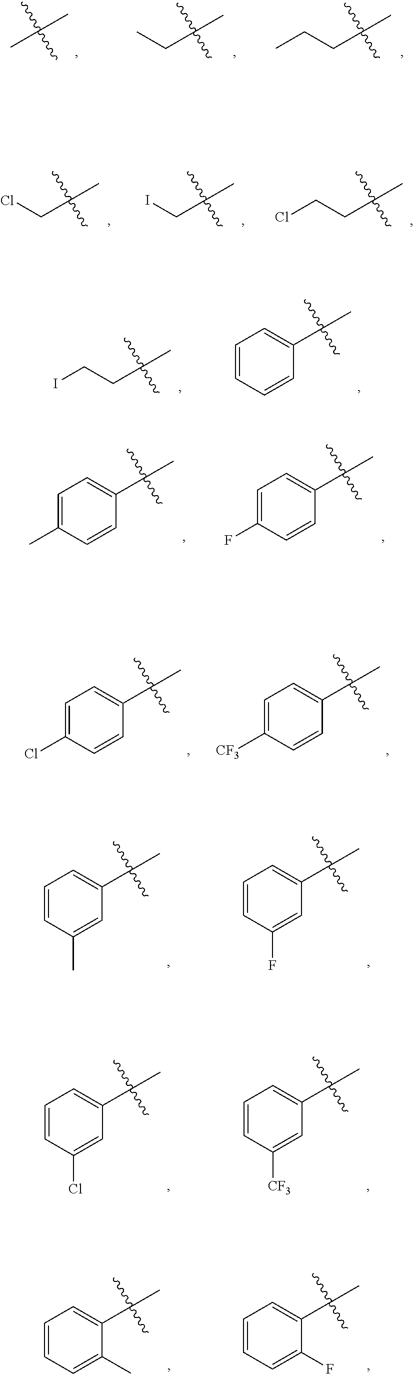

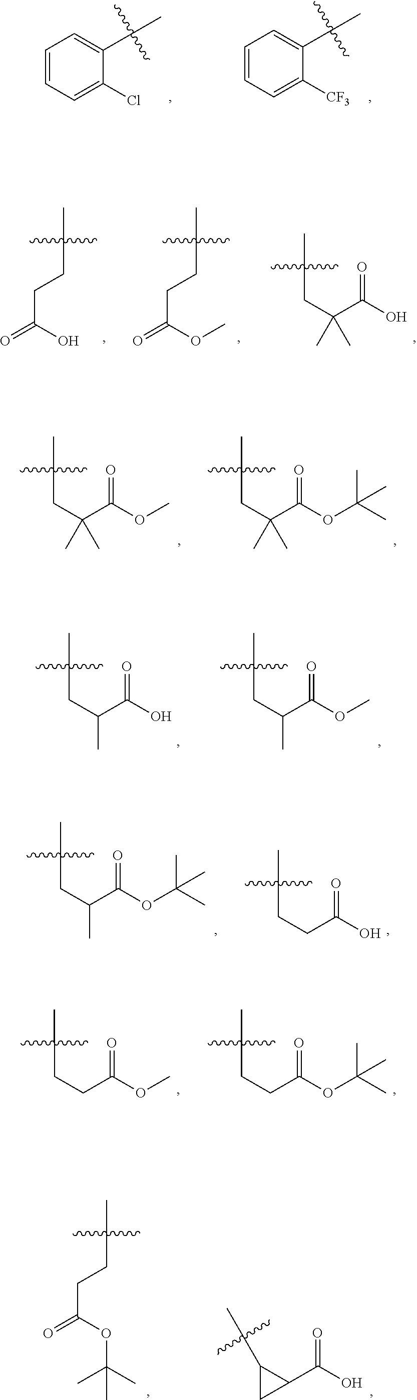

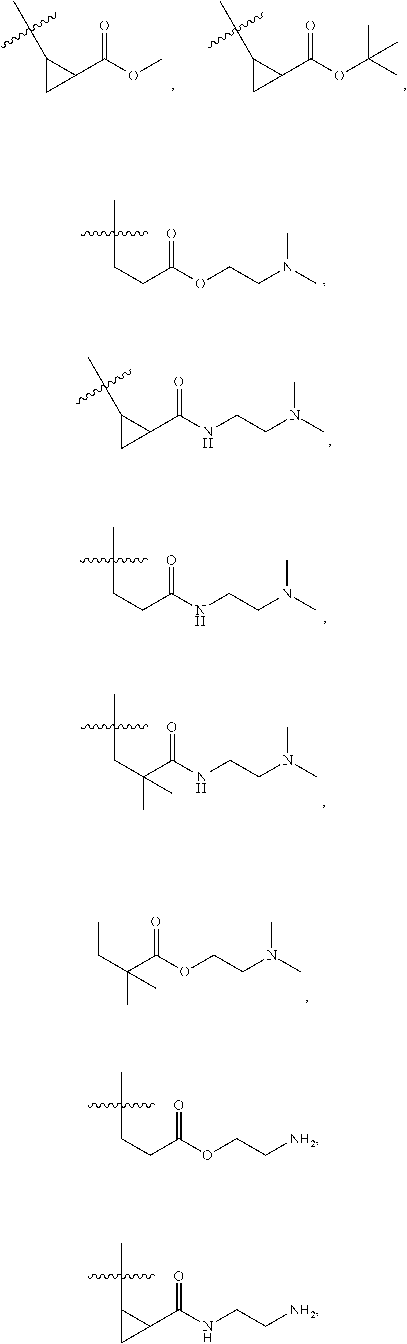

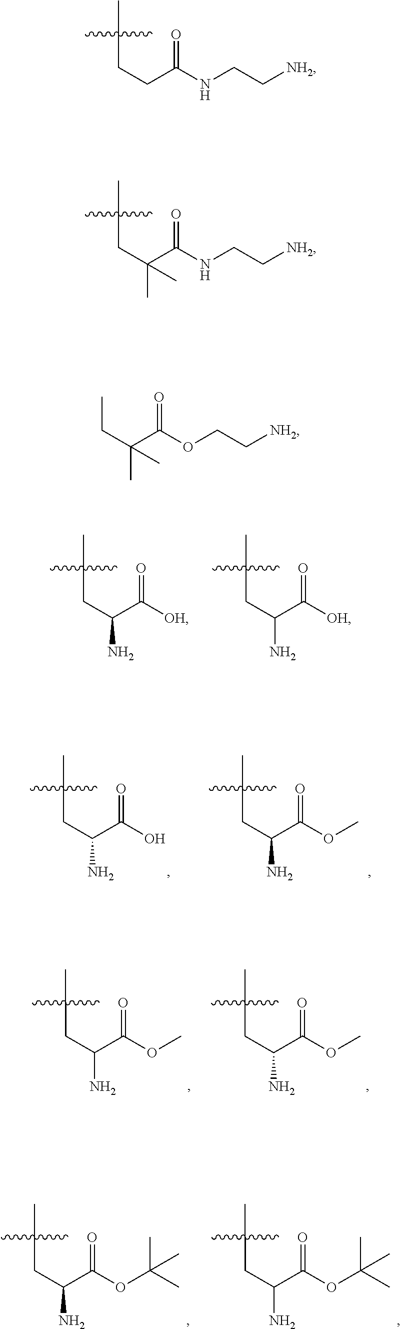

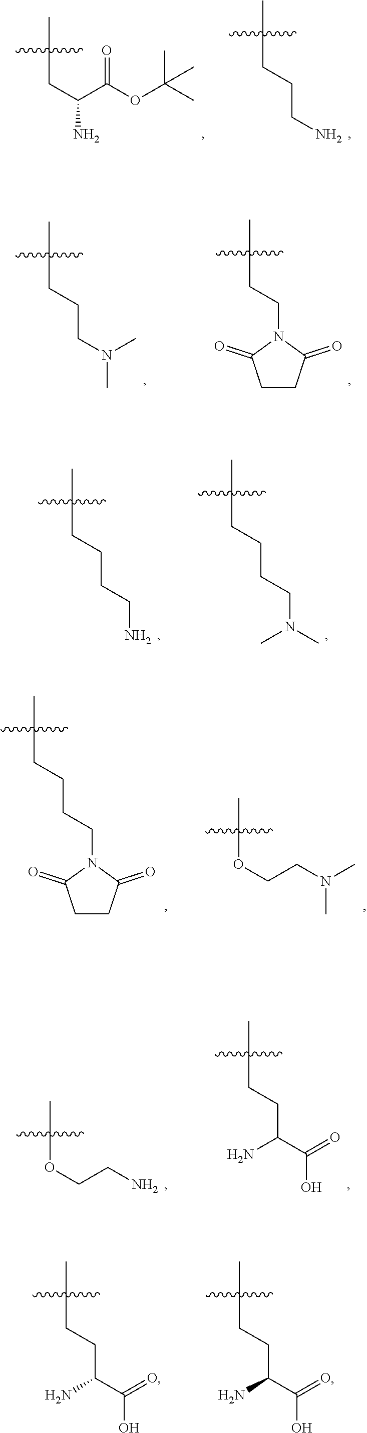

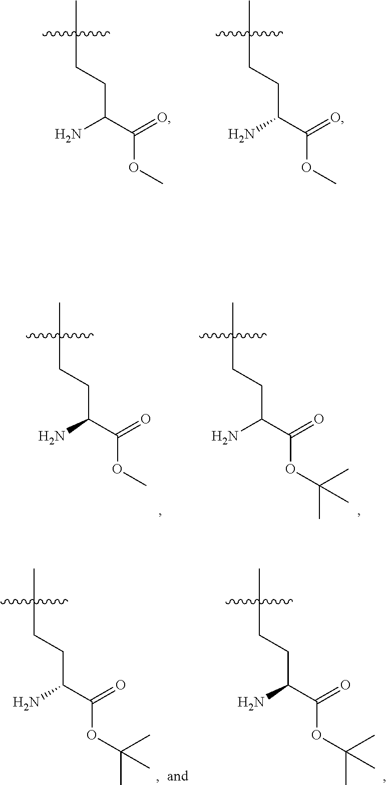

wherein [symbol] denotes the point of attachment of X to Z, or X to Y when Z is absent, in Formula (I); the point of attachment of X to the carbon atom of the —C(R^{7A})(R^{7B}) group in Formula (I-I); or the point of attachment of the R⁵ group to the carbon atom of the —C(O)— group in Formula (ID).

A compound of the present disclosure may be a compound of Formula (IE)

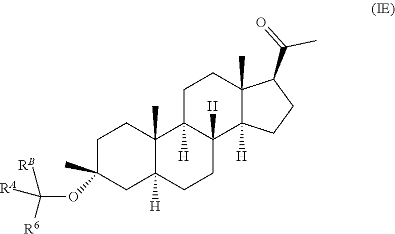

or a pharmaceutically acceptable salt, solvate, hydrate, tautomer, or stereoisomer thereof, wherein
R⁶ is alkyl, alkenyl, alkynyl, heteroalkyl, cycloalkyl, heterocyclyl, aryl, heteroaryl, haloalkyl, alkylene-cycloalkyl, alkylene-heterocyclyl, alkylene-aryl, alkylene-heteroaryl, heteroalkylene-cycloalkyl, heteroalkylene-heterocyclyl, heteroalkylene-aryl, heteroalkylene-heteroaryl, —OC(O)-alkyl, —C(O)O-alkyl, —OC(O)-heteroalkyl, —C(O)O-heteroalkyl, —N₃, —SH, or —S-alkyl (e.g., —S-Me), wherein the alkyl, alkylene, alkenyl, alkynyl, heteroalkyl, heteroalkylene, cycloalkyl, heterocyclyl, aryl, or heteroaryl is optionally substituted; and
R^{A} and R^{B} are each independently hydrogen, alkyl, heteroalkyl, cycloalkyl, heterocyclyl, aryl, or heteroaryl, wherein the alkyl, heteroalkyl, cycloalkyl, heterocyclyl, aryl, or heteroaryl is optionally substituted.

In some embodiments, the X group of Formula (I) or Formula (I-I), or the R⁶ group of Formula (IE), is a moiety selected from the group consisting of

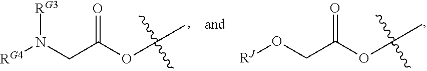

wherein
R^{G3} and R^{G4} are each independently hydrogen, alkyl, or heteroalkyl, wherein the alkyl or heteroalkyl is optionally substituted;
or R^{G3} and R^{G4}, together with the atoms to which they are attached, form a 3-7 membered cycloalkyl, heterocyclyl, aryl, or heteroaryl, wherein the cycloalkyl, heterocyclyl, aryl, or heteroaryl is optionally substituted;
R^{J} is hydrogen, alkyl, alkylene-heterocyclyl, heteroalkyl, or heterocyclyl, wherein the alkyl, alkylene, heteroalkyl, or heterocyclyl is optionally substituted;
and [symbol] denotes the point of attachment of X to Z, or X to Y when Z is absent, in Formula (I); the point of attachment of X to the carbon atom of the —C(R^{7A})(R^{7B}) group in Formula (I-I); or the point of attachment of the R⁶ group to the carbon atom of the —C(R^{A})(R^{B})— group in Formula (IE).

The compound may be a compound of Formula (I), (I-I), or (IE), wherein the X group of Formula (I) or (I-I), or the R⁶ group of Formula (IE), is a moiety selected from the group consisting of

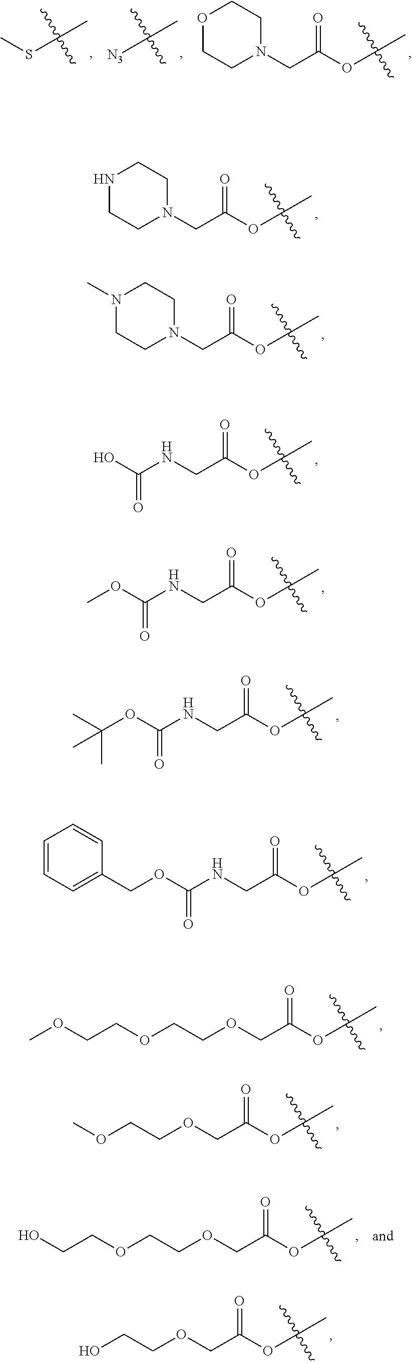

wherein [symbol] denotes the point of attachment of X to Z, or X to Y when Z is absent, in Formula (I); the point of attachment of X to the carbon atom of the —C(R^{7A})(R^{7B}) group in Formula (I-I); or the point of attachment of the R⁶ group to the carbon atom of the —C(R^{A})(R^{B})— group in Formula (IE).

A compound of the present disclosure may be a compound provided in Table 1. For example, the compound may be

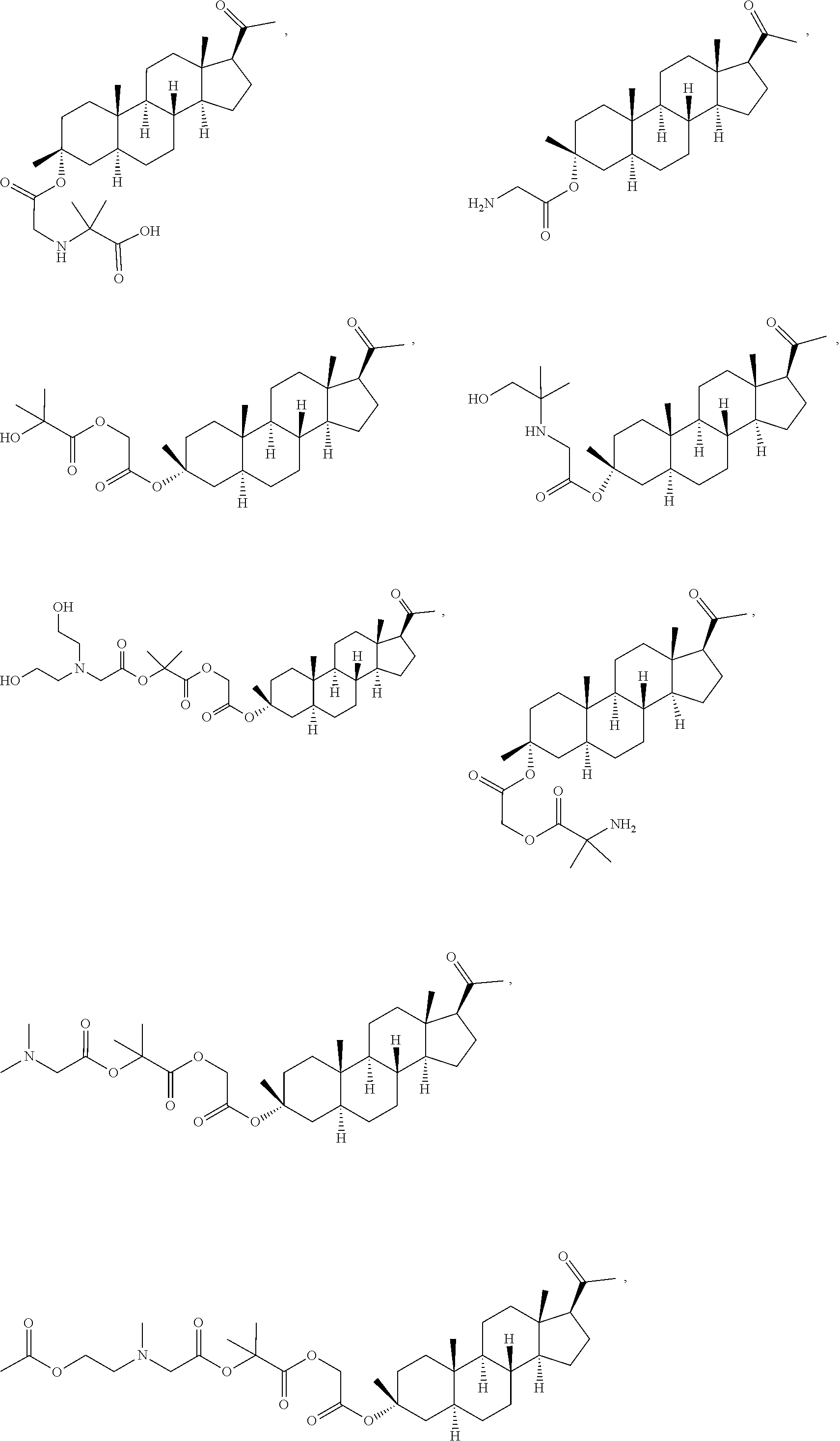

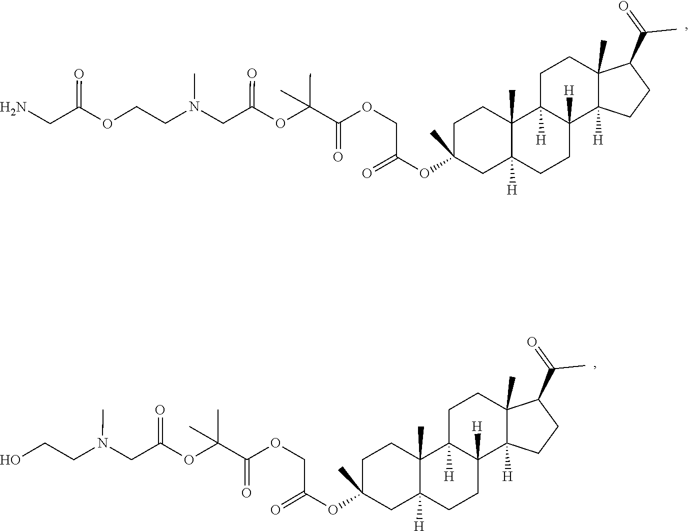

or a pharmaceutically acceptable salt (e.g., hydrochloride salt), solvate, hydrate, tautomer, or stereoisomer thereof. In some embodiments, the compound is

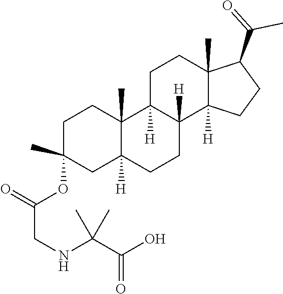

or a pharmaceutically acceptable salt (e.g., hydrochloride salt), solvate, hydrate, tautomer, or stereoisomer thereof. In some embodiments, the compound is

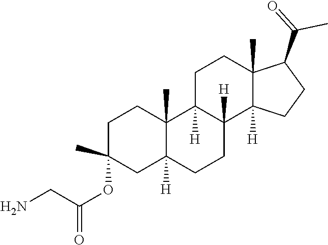

or a pharmaceutically acceptable salt (e.g., hydrochloride salt), solvate, hydrate, tautomer, or stereoisomer thereof. In certain embodiments, the compound is

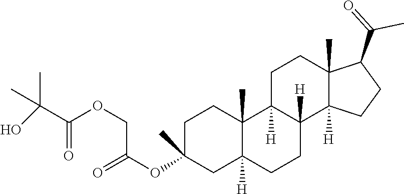

a pharmaceutically acceptable salt (e.g., hydrochloride salt), solvate, hydrate, tautomer, or stereoisomer thereof. In certain embodiments, the compound is

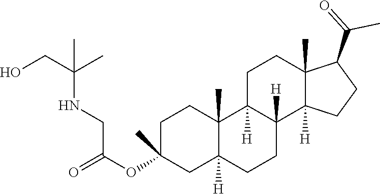

or a pharmaceutically acceptable salt (e.g., hydrochloride salt), solvate, hydrate, tautomer, or stereoisomer thereof. In certain embodiments, the compound is

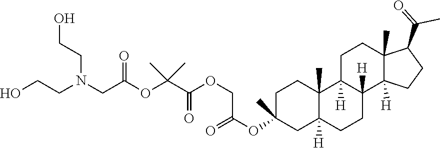

or a pharmaceutically acceptable salt (e.g., hydrochloride salt), solvate, hydrate, tautomer, or stereoisomer thereof. In certain embodiments, the compound is

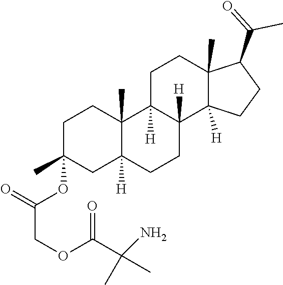

or a pharmaceutically acceptable salt (e.g., hydrochloride salt), solvate, hydrate, tautomer, or stereoisomer thereof. In certain embodiments, the compound is

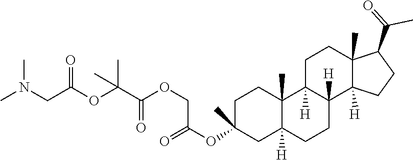

or a pharmaceutically acceptable salt (e.g., hydrochloride salt), solvate, hydrate, tautomer, or stereoisomer thereof. In certain embodiments, the compound is

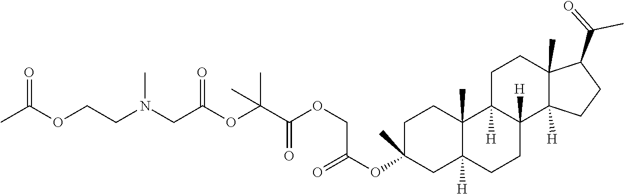

or a pharmaceutically acceptable salt (e.g., hydrochloride salt), solvate, hydrate, tautomer, or stereoisomer thereof. In certain embodiments, the compound is

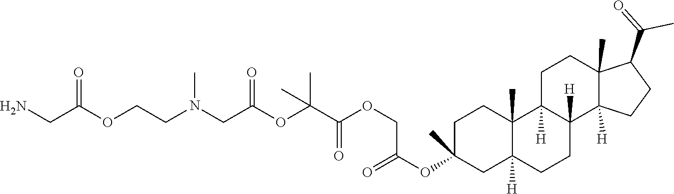

or a pharmaceutically acceptable salt (e.g., hydrochloride salt), solvate, hydrate, tautomer, or stereoisomer thereof. In certain embodiments, the compound is

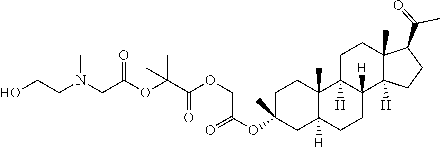

or a pharmaceutically acceptable salt (e.g., hydrochloride salt), solvate, hydrate, tautomer, or stereoisomer thereof. In certain aspects, this disclosure does not include the compound

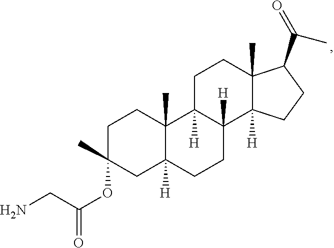

or a pharmaceutically acceptable salt (e.g., hydrochloride salt), solvate, hydrate, tautomer, or stereoisomer thereof.

In some embodiments, a compound of the present disclosure is

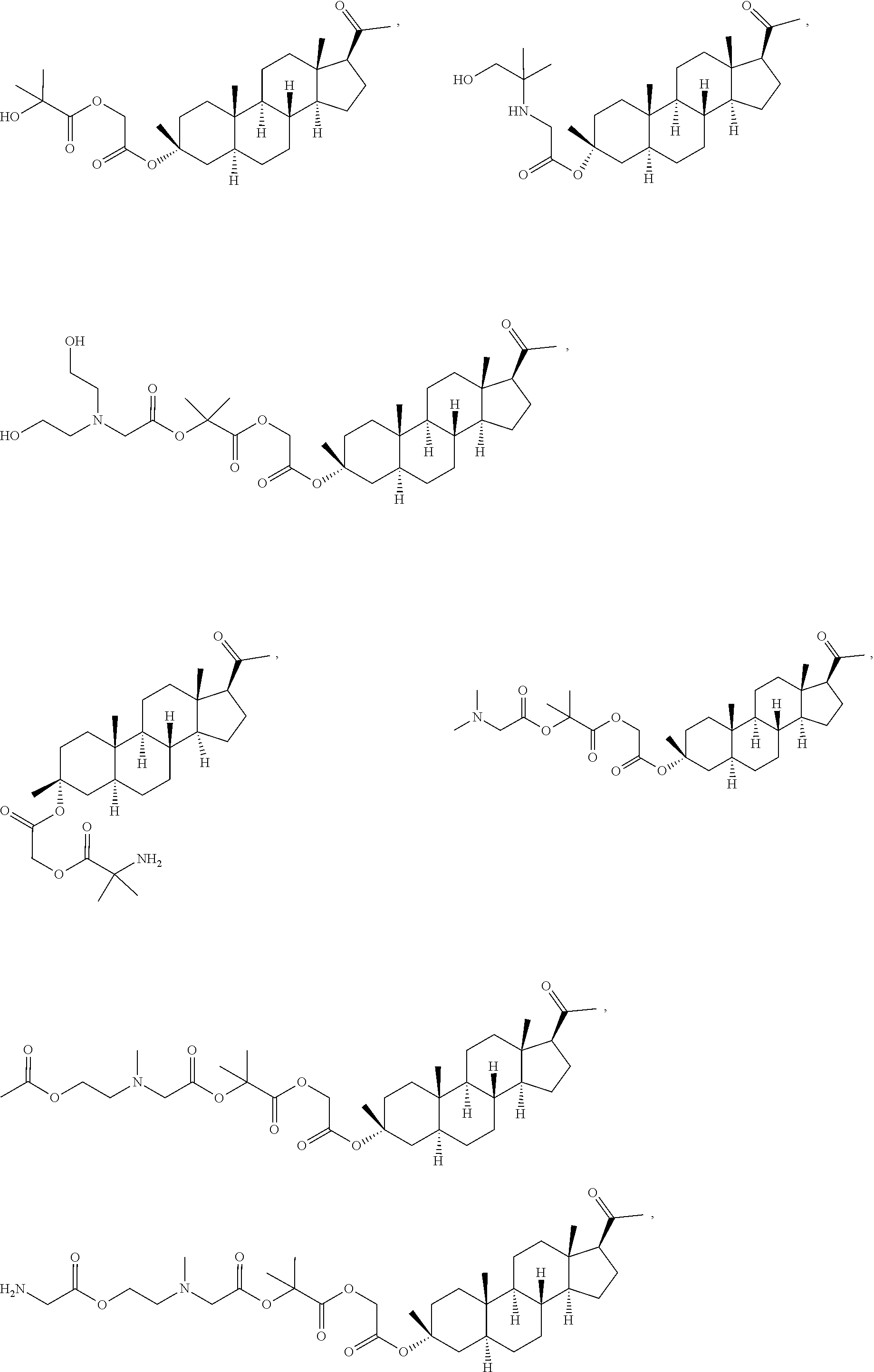

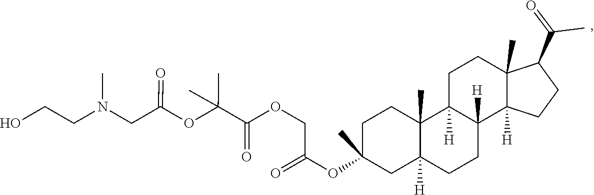

or a pharmaceutically acceptable salt (e.g., hydrochloride salt), solvate, hydrate, tautomer, or stereoisomer thereof.

A compound disclosed herein (e.g., a compound of Formulae (I), (I-I), (IA), (IA-a), (IA-b), (IB), (IC), (ID), (IE), (I-II), (I-IIa), (I-IIb), (I-IIc), (I-IId), (I-IIe), or a compound of Table 1) may have a higher aqueous solubility and/or hydrophilicity relative to ganaxolone, e.g., as determined by a suitable measurement of solubility and/or hydrophilicity, such as logS, logP, logD, topological polar surface area (TPSA), acid pKa, base pKa, or a combination thereof.

In some aspects, a compound disclosed herein may be administered to a subject without a solubility-enhancing agent such as a cyclodextrin (e.g., CAPTISOL®). For example, a compound disclosed herein may be administered to a subject without cyclodextrin (e.g., CAPTISOL®), or may be administered with relatively low amounts of cyclodextrin (e.g., CAPTISOL®) such as in a ratio of compound:cyclodextrin of less than 1:1, e.g., less than 2:1, less than 3:1, less than 4:1, less than 5:1, less than 6:1, less than 7:1, less than 8:1, less than 9:1, less than 10:1, less than 15:1, less than 20:1, less than 25:1, less than 50:1, less than 99:1, or lower. Similarly, a pharmaceutical composition disclosed herein may comprise relatively low amounts of a solubility-enhancing agent (e.g., a cyclodextrin, such as CAPTISOL®), or no solubility-enhancing agent. For example, a pharmaceutical composition disclosed herein may comprise no cyclodextrin (e.g., CAPTISOL®), or relatively low amounts of cyclodextrin (e.g., CAPTISOL®) such as less than 25 wt %, less than 20 wt %, less than 15 wt %, less than 10 wt %, less than 5 wt %, less than 4 wt %, less than 3 wt %, less than 2 wt %, less than 1 wt %, less than 0.5 wt %, of cyclodextrin in the composition.

A compound disclosed herein (e.g., a compound of Formulae (I), (I-I), (IA), (IA-a), (IA-b), (IB), (IC), (ID), (IE), (I-II), (I-IIa), (I-IIb), (I-IIc), (I-IId), (I-IIe), or a compound of Table 1) may have a higher aqueous solubility and/or hydrophilicity relative to a lipid-based derivative of ganaxolone, e.g., as determined by a suitable measurement of solubility and/or hydrophilicity, such as logS, logP, logD, topological polar surface area (TPSA), acid pKa, base pKa, or a combination thereof. The compounds of the present disclosure may also have a higher melting point relative to a lipid-based derivative of ganaxolone. For example, the compounds of the present disclosure may be solid (e.g., in powder or crystal form) at ambient temperature (e.g., about 25° C.), or at temperature between about 25 to about 100° C., or at temperature between about 25 to about 80° C., or at temperature between about 25 to about 60° C., or at temperature between about 25 to about 40° C. As such, the compounds of the present disclosure have certain advantages over lipid-based derivatives of ganaxolone, e.g., more suitable for oral or intravenous administration, greater bioavailability, and/or more easily handled or formulated into pharmaceutical compositions for oral or intravenous administration.

A compound disclosed herein (e.g., a compound of Formulae (I), (I-I), (IA), (IA-a), (IA-b), (IB), (IC), (ID), (IE), (I-II), (I-IIa), (I-IIb), (I-IIc), (I-IId), (I-IIe), or a compound of Table 1) may be converted (e.g., substantially converted) to ganaxolone by a metabolic process, e.g., following administration of the compound to a subject. For example, following administration of the compound to a subject (e.g., orally or intravenously), about 1% or more (e.g., about 2%, about 4%, about 6%, about 8%, about 10%, about 15%, about 20%, about 25%, about 30%, about 40%, about 50%, about 60%, about 70%, about 80%, about 90%, about 95%, about 99%, about 99.9%, or more) of the compound may be converted to ganaxolone, e.g., within a period of about 1 minute or longer (e.g., about 2 minutes, about 4 minutes, about 6 minutes, about 8 minutes, about 10 minutes, about 15 minutes, about 20 minutes, about 30 minutes, about 45 minutes, about 1 hour, about 1.5 hours, about 2 hours, about 3 hours, about 4 hours, about 5 hours, about 6 hours, about 7 hours, about 8 hours, about 9 hours, about 10 hours, about 11 hours, about 12 hours, about 14 hours, about 16 hours, about 18 hours, about 20 hours, about 22 hours, about 24 hours, about 36 hours, about 48 hours, or longer).

A compound disclosed herein (e.g., a compound of Formulae (I), (I-I), (IA), (IA-a), (IA-b), (IB), (IC), (ID), (IE), (I-II), (I-IIa), (I-IIb), (I-IIc), (I-IId), (I-IIe), or a compound of Table 1) may provide a higher bioavailability of ganaxolone, relative to the bioavailability of ganaxolone following administration of ganaxolone itself, e.g., by the same route of administration and at an equivalent dosage. The bioavailability of ganaxolone may be determined by measuring the serum concentration of ganaxolone (e.g., to obtain Cₘₐₓ or AUC).

A compound disclosed herein (e.g., a compound of Formulae (I), (I-I), (IA), (IA-a), (IA-b), (IB), (IC), (ID), (IE), (I-II), (I-IIa), (I-IIb), (I-IIc), (I-IId), (I-IIe), or a compound of Table 1) may provide a higher serum concentration of ganaxolone (e.g., Cₘₐₓ or AUC), relative to the serum concentration achieved following administration of ganaxolone itself, e.g., by the same route of administration and at an equivalent dosage. A compound disclosed herein (e.g., a compound of Formulae (I), (I-I), (IA), (IA-a), (IA-b), (IB), (IC), (ID), (IE), (I-II), (I-IIa), (I-IIb), (I-IIc), (I-IId), (I-IIe), or a compound of Table 1) may provide longer serum half-life of ganaxolone, e.g., following administration to a subject, relative to the serum half-life obtained following administration of ganaxolone itself, e.g., by the same route of administration and at an equivalent dosage.

For example, administering a compound disclosed herein (e.g., a compound of Formulae (I), (I-I), (IA), (IA-a), (IA-b), (IB), (IC), (ID), (IE), (I-II), (I-IIa), (I-IIb), (I-IIc), (I-IId), (I-IIe), or a compound of Table 1) orally may achieve a higher ganaxolone Cₘₐₓ, relative to administering an equivalent amount of ganaxolone orally. Administering a compound disclosed herein (e.g., a compound of Formulae (I), (I-I), (IA), (IA-a), (IA-b), (IB), (IC), (ID), (IE), (I-II), (I-IIa), (I-IIb), (I-IIc), (I-IId), (I-IIe), or a compound of Table 1) orally may achieve a higher ganaxolone AUC, relative to administering an equivalent amount of ganaxolone orally. Alternatively, administering a compound disclosed herein (e.g., a compound of Formulae (I), (I-I), (IA), (IA-a), (IA-b), (IB), (IC), (ID), (IE), (I-II), (I-IIa), (I-IIb), (I-IIc), (I-IId), (I-IIe), or a compound of Table 1) intravenously may achieve a higher ganaxolone Cₘₐₓ, relative to administering an equivalent amount of ganaxolone intravenously. Administering a compound disclosed herein (e.g., a compound of Formulae (I), (I-I), (IA), (IA-a), (IA-b), (IB), (IC), (ID), (IE), (I-II), (I-IIa), (I-IIb), (I-IIc), (I-IId), (I-IIe), or a compound of Table 1) intravenously may achieve a higher ganaxolone AUC, relative to administering an equivalent amount of ganaxolone intravenously.

Also disclosed herein are pharmaceutical compositions comprising a compound disclosed herein (e.g., a compound of Formulae (I), (I-I), (IA), (IA-a), (IA-b), (IB), (IC), (ID), (IE), (I-II), (I-IIa), (I-IIb), (I-IIc), (I-IId), (I-IIe), or a compound of Table 1) and a pharmaceutically acceptable excipient (e.g., a pharmaceutically acceptable excipient disclosed herein).

Also disclosed herein are methods of using a compound disclosed herein (e.g., a compound of Formulae (I), (I-I), (IA), (IA-a), (IA-b), (IB), (IC), (ID), (IE), (I-II), (I-IIa), (I-IIb), (I-IIc), (I-IId), (I-IIe), or a compound of Table 1). For example, disclosed herein are methods of treating a disease or disorder in a subject, comprising administering a compound disclosed herein, or a pharmaceutical composition disclosed herein, to a subject in need thereof.

For example, the method of treating a disease or disorder in a subject may comprise administering a compound of Formulae (I), (I-I), (IA), (IA-a), (IA-b), (IB), (IC), (ID), (IE), (I-II), (I-IIa), (I-IIb), (I-IIc), (I-IId), (I-IIe), or a compound of Table 1, to a subject in need thereof. The method of treating a disease or disorder in a subject may comprise administering a composition comprising a compound of Formulae (I), (I-I), (IA), (IA-a), (IA-b), (IB), (IC), (ID), (IE), (I-II), (I-IIa), (I-IIb), (I-IIc), (I-IId), (I-IIe), or a compound of Table 1, and a pharmaceutically acceptable excipient, to a subject in need thereof. The compound or composition may be administered by any suitable route of administration, e.g., a route of administration disclosed herein, such as by oral administration, or by intravenous administration.

Also disclosed herein are compositions for use in treating a disease or disorder in a subject, comprising a therapeutically effective amount of a compound disclosed herein (e.g., a compound of Formulae (I), (I-I), (IA), (IA-a), (IA-b), (IB), (IC), (ID), (IE), (I-II), (I-IIa), (I-IIb), (I-IIc), (I-IId), (I-IIe), or a compound of Table 1.

In other aspects, the present disclosure relates to a use of a compound disclosed herein (e.g., a compound of Formulae (I), (I-I), (IA), (IA-a), (IA-b), (IB), (IC), (ID), (IE), (I-II), (I-IIa), (I-IIb), (I-IIc), (I-IId), (I-IIe), or a compound of Table 1), or use of a pharmaceutical composition disclosed herein, for the production of a medicament effective for treating a disease or disorder in a subject.

The disease or disorder treated by a method, compound, composition, or medicament disclosed herein may be any suitable disease or disorder, such as a disease or disorder described herein. For example, the disease or disorder may be a neurological disorder. The disease or disorder may be a seizure disorder, epilepsy disorder, genetic epilepsy disorder, epilepsy-related disorder, central nervous system disorder, neurological disorder, or a neurodegenerative disorder. The disease or disorder may be status epilepticus (SE). The disease or disorder may be CDKL5 Deficiency Disorder. The disease or disorder may be Tuberous Sclerosis Complex. The disease or disorder may be PCDH19-related epilepsy. The disease or disorder may be Lennox-Gastaut syndrome (LGS).

### BRIEF DESCRIPTION OF THE DRAWINGS

FIGS. 1A-1B depict the pharmacokinetics of an exemplary ganaxolone prodrug disclosed herein in a dog model. FIG. 1A is a graph depicting the mean plasma concentration of Compound 111 (ng/mL) over time (h) following either intravenous (5 mg/kg) or oral (25 mg/kg) administration of Compound 111 (with vehicle) in a dog model. FIG. 1B is a graph depicting the mean plasma concentration of ganaxolone (ng/mL) over time (h) following either intravenous (5 mg/kg) or oral (25 mg/kg) administration of Compound 111 (with vehicle) in a dog model.

FIGS. 2A-2B depict the pharmacokinetics of an intravenous (IV) dose of an exemplary ganaxolone prodrug disclosed herein in a dog model. FIG. 2A is a graph depicting the mean plasma concentration (ng/mL) of ganaxolone (GNX) and Compound 224 over 8 hours following intravenous (2 mg/kg; bolus) administration of Compound 224 (with vehicle) in a dog model. FIG. 2B is a graph depicting the same pharmacokinetic study following IV administration of Compound 224, with a compressed x axis (0-1 hours).

FIGS. 3A-3B depict the pharmacokinetics of an oral (PO) dose of an exemplary ganaxolone prodrug disclosed herein in a dog model. FIG. 3A is a graph depicting the mean plasma concentration (ng/mL) of ganaxolone (GNX) and Compound 224 over 36 hours following oral (10 mg/kg; bolus) administration of Compound 224 (with vehicle) in a dog model. FIG. 3B is a graph depicting the same pharmacokinetic study following PO administration of Compound 224, with a compressed x axis (over 0-4 hours).

FIGS. 4A-4B depict the pharmacokinetics of an exemplary ganaxolone prodrug disclosed herein in a dog model. FIG. 4A is a graph depicting the mean plasma concentration of Compound 218 (ng/mL) over time (h) following either intravenous (5 mg/kg) or oral (25 mg/kg) administration of Compound 218 (with vehicle) in a dog model. FIG. 4B is a graph depicting the mean plasma concentration of ganaxolone (ng/mL) over time (h) following either intravenous (5 mg/kg) or oral (25 mg/kg) administration of Compound 218 (with vehicle) in a dog model.

### DETAILED DESCRIPTION

This disclosure relates to ganaxolone derivatives. The compounds disclosed herein can be metabolized to ganaxolone (e.g., in vivo), and may be referred to as ganaxolone prodrugs. Also disclosed herein are methods of using the compounds, methods of making the compounds, and pharmaceutical compositions and kits comprising the same.

Ganaxolone may be used for modulation of GABA_{A}, which provides the ability to treat neurological disorders including seizures and epilepsy, among other disorders disclosed herein. However, due to the poor aqueous solubility and low oral bioavailability of ganaxolone, administering therapeutically effective amounts of ganaxolone is best achieved through intravenous infusion or through oral formulations of ganaxolone, such as aqueous suspensions, that require frequent administration and/or that a large amount of the formulation be administered. The compounds disclosed herein can address this shortcoming of ganaxolone, as they have greater aqueous solubility and bioavailability compared to ganaxolone, and can be metabolized to ganaxolone after administration (e.g., oral administration). Without wishing to be bound by theory, it is believed that the greater oral bioavailability of the compounds, relative to ganaxolone, allows for the compound to be readily absorbed from the gut into the bloodstream, and the compounds are subsequently converted to ganaxolone (e.g., by metabolic processes) to achieve therapeutic concentrations of ganaxolone in the bloodstream and/or in the central nervous system (CNS).

Accordingly, this disclosure relates to compounds that comprise a ganaxolone moiety that is modified at the hydroxyl group by a cleavable moiety, e.g., an enzymatically cleavable moiety. For example, the cleavable moiety may be cleavable by an enzyme (e.g., an esterase) or through other mechanisms (e.g., direct chemical mechanisms). For example, following administration of a compound disclosed herein, the cleavable moiety may be cleaved (e.g., enzymatically) to provide ganaxolone. Compounds or compositions of the present disclosure may be administered, without limitation, orally, subcutaneously, intramuscularly, intravenously, intradermally, by inhalation, topically, rectally, nasally, buccally, vaginally, or by an implanted reservoir. In preferred embodiments, the compounds or compositions of the present disclosure are suitable for oral administration. In other preferred embodiments, the compounds or compositions of the present disclosure are suitable for intravenous administration. The disclosure also relates to methods for treating a neurological disorder, such as epilepsy or seizures. The methods disclosed herein can comprise orally administering to a subject in need thereof a therapeutically effective amount of a compound disclosed herein. Alternatively, the methods disclosed herein can comprise intravenously administering to a subject in need thereof a therapeutically effective amount of a compound disclosed herein.

### Definitions

All publications (e.g., scientific journal articles, patent publications, and the like) cited in this disclosure are incorporated by reference in their entirety. To the extent the material incorporated by reference contradicts or is inconsistent with this specification, the specification will supersede any such material. The citation of any references herein is not an admission that such references are prior art to the present disclosure. Various terms relating to aspects of the description are used throughout the specification and claims. Such terms are to be given their ordinary meaning in the art unless otherwise indicated. Other specifically defined terms are to be construed in a manner consistent with the definitions provided herein.

As used herein, the term “alkyl” refers to a radical of a saturated hydrocarbon group having 1 to 18 carbon atoms (C₁₋₁₈-alkyl), such as 1 to about 12 carbon atoms (C₁₋₁₂-alkyl), or 1 to about 6 carbon atoms (C₁₋₆-alkyl). An alkyl group can be straight chain or branched chain hydrocarbon group. Examples of alkyl groups include methyl, ethyl, propyl, isopropyl, butyl, iso-butyl, sec-butyl, tert-butyl, pentyl, hexyl, cyclopropyl, cyclobutyl, cyclopentyl, cyclohexyl, norbornyl, and the like. Throughout this disclosure, abbreviations that are well known in the art to describe various alkyl groups or their derivatives may be used, such as Me (methyl), Et (ethyl), Pr (propyl) including iPr (isopropyl), Bu (butyl) including tBu (tert-butyl), Bn (benzyl). Each instance of an alkyl group may be independently optionally substituted, i.e., unsubstituted (an “unsubstituted alkyl”) or substituted (a “substituted alkyl”) with one or more substituents e.g., for instance from 1 to 5 substituents, 1 to 3 substituents, or 1 substituent.

As used herein, the term “alkenyl” refers to a radical of a straight-chain or branched hydrocarbon group having from 2 to 18 carbon atoms, one or more carbon-carbon double bonds, and no triple bonds. For example, an alkenyl group may have 2 to 8 carbon atoms (C₂₋₈-alkenyl), 2 to 6 carbon atoms (C₂₋₆-alkenyl), 2 to 5 carbon atoms (C₂₋₅-alkenyl), 2 to 4 carbon atoms (C₂₋₄-alkenyl), or 2 to 3 carbon atoms (C₂₋₃-alkenyl). The one or more carbon-carbon double bonds can be internal (such as in 2-butenyl) or terminal (such as in 1-butenyl). Examples of alkenyl groups include ethenyl, 1-propenyl, 2-propenyl, 1-butenyl, 2-butenyl, butadienyl, pentenyl, pentadienyl, hexenyl, heptenyl, octenyl, octatrienyl, and the like. Each instance of an alkenyl group may be independently optionally substituted, i.e., unsubstituted (an “unsubstituted alkenyl”) or substituted (a “substituted alkenyl”) with one or more substituents, e.g., for instance from 1 to 5 substituents, 1 to 3 substituents, or 1 substituent.

As used herein, the term “alkynyl” refers to a radical of a straight-chain or branched hydrocarbon group having from 2 to 18 carbon atoms, and one or more carbon-carbon triple bonds. The alkynyl group may have 2 to 8 carbon atoms (C₂₋₈-alkynyl), 2 to 6 carbon atoms (C₂₋₆-alkynyl), 2 to 5 carbon atoms (C₂₋₅-alkynyl), 2 to 4 carbon atoms (C₂₋₄-alkynyl), or 2 to 3 carbon atoms (C₂₋₃-alkynyl). The one or more carbon-carbon triple bonds can be internal (such as in 2-butynyl) or terminal (such as in 1-butynyl). Examples of alkynyl groups include ethynyl, 1-propynyl, 2-propynyl, 1-butynyl, 2-butynyl, and the like. Each instance of an alkynyl group may be independently optionally substituted, i.e., unsubstituted (an “unsubstituted alkynyl”) or substituted (a “substituted alkynyl”) with one or more substituents, e.g., for instance from 1 to 5 substituents, 1 to 3 substituents, or 1 substituent

As used herein, the term “heteroalkyl” refers to a non-cyclic stable straight or branched chain, or combinations thereof, including at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) O, N, P, S, and Si may be placed at any position of the heteroalkyl group. Where “heteroalkyl” is recited, followed by recitations of specific heteroalkyl groups, such as —(CH₂)—C(O)—OH, —N(R^{G1})(R^{G2}), or the like, it will be understood that the terms heteroalkyl and —(CH₂)—C(O)—OH, —N(R^{G1})(R^{G2}) are not redundant or mutually exclusive. Rather, the specific heteroalkyl groups are recited to add clarity. Thus, the term “heteroalkyl” should not be interpreted herein as excluding specific heteroalkyl groups, such as —(CH₂)—C(O)—OH, —N(R^{G1})(R^{G2}), or the like. Each instance of a heteroalkyl group may be independently optionally substituted, i.e., unsubstituted (an “unsubstituted heteroalkyl”) or substituted (a “substituted heteroalkyl”) with one or more substituents, e.g., for instance from 1 to 5 substituents, 1 to 3 substituents, or 1 substituent.

As used herein, the term “haloalkyl” refers to a non-cyclic straight or branched chain, or combinations thereof, including at least one carbon atom and at least one halogen (e.g., F, Cl, Br, and I). The halogen atom(s) may be placed at any position of the haloalkyl group. Exemplary haloalkyl groups include, but are not limited to: —CF₃, —CCl₃, —CH₂—CF₃, —CH₂—CCl₃, —CH₂—Cl, and —CH₂—I. Each instance of a haloalkyl group may be independently optionally substituted, i.e., unsubstituted (an “unsubstituted haloalkyl”) or substituted (a “substituted haloalkyl”) with one or more substituents, e.g., for instance from 1 to 5 substituents, 1 to 3 substituents, or 1 substituent.

As used herein, the term “alkoxy” refers to a group of formula —O-alkyl. The term “alkoxy” or “alkoxyl” includes substituted and unsubstituted alkyl, alkenyl and alkynyl groups covalently linked to an oxygen atom. Examples of alkoxy groups or alkoxyl radicals include, but are not limited to, methoxy, ethoxy, isopropyloxy, propoxy, butoxy, and pentoxy groups. Examples of substituted alkoxy groups include halogenated alkoxy groups. The alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy and trichloromethoxy.

As used herein, the term “aryl,” refers to stable aromatic ring system, that may be monocyclic or polycyclic, of which all the ring atoms are carbon, and which may be substituted or unsubstituted. The aromatic ring system may have, for example, 3-7 ring atoms. Examples include phenyl (Ph), naphthyl, anthracyl, and the like. Each instance of an aryl group may be independently optionally substituted, i.e., unsubstituted (an “unsubstituted aryl”) or substituted (a “substituted aryl”) with one or more substituents.

As used herein, the term “heteroaryl” refers to an aryl group that includes one or more ring heteroatoms. For example, a heteroaryl can include a stable 5-, 6-, or 7-membered monocyclic or 7-, 8-, or 9-membered bicyclic aromatic heterocyclic ring which consists of carbon atoms, and one or more heteroatoms independently selected from the group consisting of nitrogen, oxygen and sulfur. The nitrogen atom may be substituted or unsubstituted (e.g., N or NR″ wherein R″ is H or another substituent, as defined). Examples of heteroaryl groups include pyrrole, furan, indole, thiophene, thiazole, isothiazole, imidazole, triazole, tetrazole, pyrazole, oxazole, isoxazole, pyridine, pyrazine, pyridazine, pyrimidine, and the like.

As used herein, the term “cycloalkyl” refers to a radical of a non-aromatic cyclic hydrocarbon group having from three to ten carbon atoms in the cyclic structure, and zero heteroatoms in the non-aromatic cyclic structure. Cycloalkyl can include cyclobutyl, cyclopropyl, cyclopentyl, cyclohexyl and the like. The cycloalkyl group can be either monocyclic (“monocyclic cycloalkyl”) or contain a fused, bridged or spiro ring system such as a bicyclic system (“bicyclic cycloalkyl”) and can be saturated or can be partially unsaturated. “Cycloalkyl” also includes ring systems wherein the cycloalkyl ring, as defined above, is fused with one or more aryl groups wherein the point of attachment is on the cycloalkyl ring, and in such instances, the number of carbons continue to designate the number of carbons in the cycloalkyl ring system. Each instance of a cycloalkyl group may be independently optionally substituted, i.e., unsubstituted (an “unsubstituted cycloalkyl”) or substituted (a “substituted cycloalkyl”) with one or more substituents.

As used herein, the term “heterocyclyl” refers to a radical of a 3- to 10-membered non-aromatic cyclic structure comprising atoms of at least two different elements in the ring or rings (i.e., a radical of a heterocyclic ring). The heteroatom may be selected from nitrogen, oxygen, sulfur, boron, phosphorous, and silicon. In heterocyclyl groups that contain one or more nitrogen atoms, the point of attachment can be a carbon or nitrogen atom, as valency permits. A heterocyclyl group can be either monocyclic (“monocyclic heterocyclyl”) or a fused, bridged, or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more cycloalkyl groups wherein the point of attachment is either on the cycloalkyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Each instance of heterocyclyl may be independently optionally substituted, i.e., unsubstituted (an “unsubstituted heterocyclyl”) or substituted (a “substituted heterocyclyl”) with one or more substitutents. Additional reference is made to: Oxford Dictionary of Biochemistry and Molecular Biology, Oxford University Press, Oxford, 1997 as evidence that heterocyclic ring is a term well-established in field of organic chemistry.

The terms “alkylene,” “alkenylene,” “alkynylene,” or “heteroalkylene,” alone or as part of another substituent, mean, unless otherwise stated, a divalent radical derived from an alkyl, alkenyl, alkynyl, or heteroalkyl, respectively. For instance, the term “alkenylene,” by itself or as part of another substituent, means, unless otherwise stated, a divalent radical derived from an alkene. In the case of heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— may represent both —C(O)₂R′- and —R′C(O)₂—. Each instance of an alkylene, alkenylene, alkynylene, or heteroalkylene group may be independently optionally substituted, i.e., unsubstituted (an “unsubstituted alkylene”) or substituted (a “substituted heteroalkylene”) with one or more substituents.

As used herein, the terms “cycloalkylene,” “heterocyclylene,” “arylene,” and “heteroarylene,” alone or as part of another substituent, mean a divalent radical derived from a cycloalkyl, heterocyclyl, aryl, and heteroaryl, respectively. Each instance of a cycloalkylene, heterocyclylene, arylene, or heteroarylene may be independently optionally substituted, i.e., unsubstituted (an “unsubstituted arylene”) or substituted (a “substituted heteroarylene”) with one or more substituents.

As used herein, the term “dipeptide” refers to a moiety that is composed of two amino acid residues. For example, a dipeptide disclosed herein may be leucine-leucine (Leu-Leu), glycine-alanine (Gly-Ala), glycine-valine (Gly-Val). The dipeptide may be substituted or modified at either amino acid residue, or both, for example to attach the dipeptide at a C-terminus or N-terminus, or both, to another group or atom. For example, a compound disclosed herein may comprise a dipeptide that has a free N-terminus (i.e., —NH₂), and a C-terminus that is modified to form an ester group, or a compound disclosed herein may comprise a dipeptide that has an N-terminus modified to form a carbamate group, and a C-terminus that is modified to form an ester group.

As used herein, the term “cyano” or “—CN” refer to a substituent having a carbon atom joined to a nitrogen atom by a triple bond, e.g., C≡N.

As used herein, the term “halo” or “halogen” refers to F, Cl, Br, or I.

As used herein, the term “hydroxy” refers to a group of formula —OH.

As used herein, the term “nitro” refers to a substituent having two oxygen atoms bound to a nitrogen atom, e.g., —NO₂.

As used herein, the term “oxo” refers to an oxygen group which is double bonded to another atom, e.g., carbon. For example, “oxo” refers to the ═O in a carbonyl group (C═O). Oxo may be used in reference to a compound that is substituted with a double bonded oxygen, for example “oxo” may be used to refer to the oxygen substituent in —CH₂—C(O)—CH₃.

As used herein, the term “substituted” in reference to a substituted alkyl, substituted alkylene, substituted alkenyl, substituted alkenylene, substituted alkynyl, substituted alkynylene, substituted heteroalkyl, substituted heteroalkylene, substituted heteroalkenyl, substituted heteroalkenylene, substituted heteroalkynyl, substituted heteroalkynylene, substituted cycloalkyl, substituted cycloalkylene, substituted heterocyclyl, substituted heterocyclylene, substituted aryl, substituted arylene, substituted heteroaryl, substituted heteroarylene, and the like, refers to alkyl, alkylene, alkenyl, alkenylene, alkynyl, alkynylene, heteroalkyl, heteroalkylene, heteroalkenyl, heteroalkenylene, heteroalkynyl, heteroalkynylene, cycloalkyl, cycloalkylene, heterocyclyl, heterocyclylene, aryl, arylene, heteroaryl, heteroarylene moieties, and the like, respectively, having substituents replacing one or more hydrogen atoms on one or more carbons or heteroatoms of the moiety. In general, the term “substituted” means that at least one hydrogen present on a group (e.g., a hydrogen bonded to carbon or nitrogen atom of said group) is replaced with a suitable substituent, such as a substituent described herein. Such substituents can be any suitable substituent including, for example, alkyl (e.g., C₁-C₆ alkyl), alkenyl (e.g., C₂-C₆ alkenyl), alkynyl (e.g., C₂-C₆ alkynyl), heteroalkyl (e.g., C₁-C₆ heteroalkyl), haloalkyl (e.g., C₁-C₆ haloalkyl, e.g., —CH₂Cl, —CH₂I, —(CH₂)₂Cl, —(CH₂)₂I, or —CF₃), cycloalkyl (e.g., C₃-C₈ cycloalkyl, e.g., cyclopropyl, cyclobutyl, cyclopentyl, or cyclohexyl), heterocyclyl (e.g., C₃-C₈ heterocyclyl, e.g., morpholinyl, piperazinyl, N-methylpiperazinyl, azetidinyl, or N-Boc-azetidinyl), alkylaryl (e.g., benzyl), aryl (e.g., phenyl), heteroaryl (e.g., pyrrolyl, imidazolyl, or indolyl), halogen (e.g., —F, —Cl, —Br, or —I), hydroxyl, alkylcarbonyloxy, arylcarbonyloxy, alkoxy (e.g., —OMe, —OEt, or —OBn), alkoxycarbonyloxy, aryloxycarbonyloxy, carboxylate, alkylcarbonyl, arylcarbonyl, alkoxycarbonyl, amino (e.g., —NH₂, —NHMe, or —NMe₂), aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl, and ureido), amidino, imino, sulfhydryl, alkylthio, arylthio, thiocarboxylate, sulfate, sulfinyl, sulfonyl, alkylsulfinyl, sulfonato, sulfamoyl, sulfonamido, oxo, nitro, trifluoromethyl, cyano, azido, alkyl-cycloalkyl, alkyl-heterocyclyl, alkylheteroaryl, —C(O)OH, —C(O)O-alkyl (e.g., —C(O)O-tBu or —C(O)OMe), —C(O)O-heteroalkyl, —C(O)O-alkylaryl, —OC(O)O-alkyl, —OC(O)O-heteroalkyl, —OC(O)O-alkylaryl, —C(O)NH₂, —C(O)NH-alkyl, —C(O)NH-heteroalkyl, —C(O)NH-alkylaryl, —NHC(O)O-alkyl (e.g., —NHC(O)OtBu), —NHC(O)O-heteroalkyl, —NHC(O)O-alkylaryl (e.g., —NH-Cbz), —C(O)N(alkyl)₂, —C(O)N(alkyl)(heteroalkyl), —C(O)N(heteroalkyl)₂, —N(alkyl)C(O)O-alkyl (e.g., N(Me)C(O)OtBu), —N(alkyl)C(O)O— heteroalkyl, —N(alkyl)C(O)O-alkylaryl, —N(alkyl)C(O)N(alkyl)₂, —N(alkyl)C(O)N(alkyl)(heteroalkyl), —N(alkyl)C(O)N(heteroalkyl)₂, —N(alkyl)C(O)NH-alkylaryl, ═NH, or ═N-alkyl. Cyclic groups (e.g., cycloalkyl, heterocyclyl, aryl, and heteroaryl) can be substituted at one or more ring positions with any suitable substituent, such as one of the substituents listed above.

As used herein, the term “protecting group” refers to a group that acts to temporarily block a particular functional moiety, e.g., O, S, or N, so that a reaction can be carried out selectively at another reactive site in a multifunctional compound. It will be appreciated by one of ordinary skill in the art that the synthetic methods and compounds described herein may utilize a variety of protecting groups. Protecting groups may be introduced and removed at appropriate stages during the synthesis of a compound using methods that are known to one of ordinary skill in the art. The protecting groups are applied according to standard methods of organic synthesis as described in the literature (Theodora W. Greene and Peter G. M. Wuts (2007) Protecting Groups in Organic Synthesis, 4^{th} edition, John Wiley and Sons, incorporated by reference in its entirety).

Exemplary protecting groups include, but are not limited to, oxygen, sulfur, nitrogen and carbon protecting groups. For example, oxygen protecting groups include, but are not limited to, methyl ethers, substituted methyl ethers (e.g., MOM (methoxymethyl ether), MTM (methylthiomethyl ether), BOM (benzyloxymethyl ether), PMB (p-methoxybenzyl ether), optionally substituted ethyl ethers, optionally substituted benzyl ethers, silyl ethers (e.g., TMS (trimethylsilyl ether), TES (triethylsilylether), TIPS (triisopropylsilyl ether), TBDMS (t-butyldimethylsilyl ether), tribenzyl silyl ether, TBDPS (t-butyldiphenyl silyl ether), esters (e.g., formate, acetate, benzoate (Bz), trifluoroacetate, dichloroacetate) carbonates, cyclic acetals and ketals. In addition, nitrogen or amino protecting groups include, but are not limited to, carbamates (including methyl, ethyl and substituted ethyl carbamates (e.g., Boc or Troc), amides, cyclic imide derivatives, N-Alkyl and N-Aryl amines, imine derivatives, and enamine derivatives, etc. Amino protecting groups include, but are not limited to fluorenylmethyloxycarbonyl (Fmoc), tert-butyloxycarbonyl (Boc), carboxybenzyl (Cbz), acetamide, trifluoroacetamide, etc. Certain other exemplary protecting groups are detailed herein, however, it will be appreciated that the present disclosure is not intended to be limited to these protecting groups; rather, a variety of additional equivalent protecting groups may be utilized according to methods known to one skilled in the art.

The compounds provided herein may exist in one or more particular geometric, optical, enantiomeric, diastereomeric, epimeric, stereoisomeric, tautomeric, conformational, or anomeric forms, including but not limited to: cis- and trans-forms; E- and Z-forms; endo- and exo-forms; R—, S—, and meso-forms; D- and L-forms; d- and 1-forms; (+) and (−) forms; keto-, enol-, and enolate-forms; syn- and anti-forms; synclinal- and anticlinal-forms; α- and β-forms; axial and equatorial forms; boat-, chair-, twist-, envelope- and half chair-forms; and combinations thereof, hereinafter collectively referred to as “isomers” (or “isomeric forms”).

Compounds described herein may comprise one or more asymmetric centers, and thus can exist in various isomeric forms, e.g., enantiomers and/or diastereomers. For example, the compounds described herein can be in the form of an individual enantiomer, diastereomer, or geometric isomer, or can be in the form of a mixture of stereoisomers, including racemic mixtures and mixtures enriched in one or more stereoisomer. In an embodiment, the stereochemistry depicted in a compound is relative rather than absolute. Isomers can be isolated from mixtures by methods known to those skilled in the art, including chiral high-pressure liquid chromatography (HPLC) and the formation and crystallization of chiral salts; or preferred isomers can be prepared by asymmetric syntheses. This disclosure additionally encompasses compounds described herein as individual isomers substantially free of other isomers, and alternatively, as mixtures of various isomers.

As used herein, a pure enantiomeric compound is substantially free from other enantiomers or stereoisomers of the compound (i.e., in enantiomeric excess). In other words, an “S” form of the compound is substantially free of the “R” form of the compound, and is thus, in enantiomeric excess of the “R” form. Or, using another notation, the (+) form of a compound is substantially free of the (−) form of the compound, and is thus, in enantiomeric excess of the (−) form. The term “enantiomerically pure,” or “enantiopure,” or “pure enantiomer” denotes that the compound comprises more than 75 wt %, more than 80 wt %, more than 85 wt %, more than 90 wt %, more than 91 wt %, more than 92 wt %, more than 93 wt %, more than 94 wt %, more than 95 wt %, more than 96 wt %, more than 97 wt %, more than 98 wt %, more than 99 wt %, more than 99.5 wt %, more than 99.9 wt %, or more, of the enantiomer. The weights may be based upon total weight of all enantiomers or stereoisomers of the compound.

Enantiomerically pure compounds disclosed herein may be present with other active or inactive components. For example, a pharmaceutical composition comprising an enantiomerically pure R-compound can comprise, for example, about 90% excipient and about 10% enantiomerically pure R-compound. A diastereomerically pure compound disclosed herein can also be present with other active or inactive components. For example, a pharmaceutical composition comprising a diastereomerically pure exo compound can comprise, for example, about 90% excipient and about 10% diastereomerically pure exo compound.

The active ingredient of a composition disclosed herein can be formulated with little or no excipient or carrier.

Compounds disclosed herein may also comprise one or more isotopic substitutions. For example, H may be in any isotopic form, including ¹H, ²H (D or deuterium), ³H (T or tritium); C may be in any isotopic form, including ¹²C, ¹³C, and ¹⁴C; O may be in any isotopic form, including ¹⁶O and ¹⁸O; N may be in any isotopic form, including ¹⁴N and ¹⁵N; F may be in any isotopic form, including ¹⁸F, ¹⁹F, and the like.

The term “pharmaceutically acceptable salt” as used herein refers to a salts of the compound prepared with relatively nontoxic acids or bases, depending on the particular substituents found on the respective compound. When compounds of the present disclosure contain relatively acidic functionalities, base addition salts can be obtained by contacting the neutral form of such compounds with a sufficient amount of the desired base, either neat or in a suitable solvent (e.g., an inert solvent). Examples of pharmaceutically acceptable base addition salts include sodium, potassium, calcium, ammonium, organic amino, magnesium salt, or a similar salt. When compounds of the present invention contain relatively basic functionalities, acid addition salts can be obtained by contacting the neutral form of such compounds with a sufficient amount of the desired acid, either neat or in a suitable solvent (e.g., an inert solvent). Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galacturonic acids and the like. Certain compounds of the present disclosure can contain both basic and acidic functionalities that allow the compounds to be converted into either base or acid addition salts. These salts may be prepared by methods known to those skilled in the art. Other pharmaceutically acceptable carriers known to those of skill in the art are suitable for the present invention.

The term “solvate” as used herein refers to forms of a compound that are associated with a solvent, usually by a solvolysis reaction. This physical association may include hydrogen bonding. Conventional solvents include water, methanol, ethanol, acetic acid, dimethylsulfoxide (DMSO), tetrahydrofuran (THF), diethyl ether, and the like. Compounds of the present disclosure may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolable solvates. Representative solvates include hydrates, ethanolates, and methanolates.

The term “hydrate” as used herein refers to a compound which is associated with water. Typically, the number of the water molecules contained in a hydrate of a compound is in a definite ratio to the number of the compound molecules in the hydrate. Therefore, a hydrate of a compound may be represented, for example, by the general formula R·xH₂O, wherein R is the compound and wherein x is a number greater than 0. A given compound may form more than one type of hydrates, including, e.g., monohydrates (x is 1), lower hydrates (x is a number greater than 0 and smaller than 1, e.g., hemihydrates (R·0.5H₂O)), and polyhydrates (x is a number greater than 1, e.g., dihydrates (R·2H₂O) and hexahydrates (R·6H₂O)).

The term “tautomer” as used herein refers to compounds that are interchangeable forms of a particular compound structure, and that vary in the displacement of hydrogen atoms and electrons. Thus, two structures may be in equilibrium through the movement of R electrons and an atom (usually H). For example, enols and ketones are tautomers because they are rapidly interconverted by treatment with either acid or base. Another example of tautomerism is the aci- and nitro-forms of phenylnitromethane that are likewise formed by treatment with acid or base. Tautomeric forms may be relevant to the attainment of the optimal chemical reactivity and biological activity of a compound of interest.

### Other Definitions

The articles “a” and “an” are used herein to refer to one or more than one (i.e., to at least one) of the grammatical object of the article. By way of example, “an element” means one element or more than one element.

The term “about” when referring to a measurable value such as an amount, a temporal duration, and the like, is meant to encompass variations of 20% or less, or in some instances±15% or less, or in some instances±10% or less, or in some instances±5% or less, or in some instances±1% or less, or in some instances±0.1% or less, from the specified value, as such variations are appropriate, e.g., to perform the disclosed methods.

The phrase “and/or” as used herein should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B,” when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

The term “effective amount” as used herein refers to an amount of a compound, or a composition or formulation of the compound, described herein (e.g., a ganaxolone derivative or prodrug, or pharmaceutical composition thereof), which is sufficient to achieve a desired result under the conditions of administration. For example, an effective amount of a compound disclosed herein, or a composition or formulation of the compound, for the treatment of an epilepsy disorder is an amount that can manage seizure activity, suppress seizure, allow the patient to recover from a hyperexcitable state, prevent seizure-relapse, or can provide continued suppression of seizure. A skilled clinician can determine appropriate dosing based on a variety of considerations including the severity of the disorder, the subject's age, weight, general health and other considerations. Typically compounds are administered in an amount of about 0.01 mg to about 1800 mg.

As used herein, the term “pharmaceutical compositions” are compositions comprising at least one active agent (which may be any compound disclosed herein or a pharmaceutically acceptable salt, solvate, or hydrate thereof, such as any ganaxolone derivative or prodrug disclosed herein) and at least one other substance, such as an excipient (e.g., a pharmaceutically acceptable excipient). Pharmaceutical compositions optionally contain one or more additional active agents. When specified, pharmaceutical compositions meet the U.S. FDA's GMP (good manufacturing practice) standards for human or non-human drugs. “Pharmaceutical combinations” are combinations of at least two active agents which may be combined in a single dosage form or provided together in separate dosage forms with instructions that the active agents are to be used together to treat a disorder, such as a neurological disorder, e.g., a seizure disorder, an epilepsy disorder, a genetic epilepsy disorder, an epilepsy-related disorder, a central nervous system disorder, a neurological disorder, or a neurodegenerative disorder, such as status epilepticus, CDKL5 Deficiency Disorder, Tuberous Sclerosis Complex, PCDH19-related epilepsy, or Lennox-Gastaut syndrome (LGS).

The term “pharmaceutically acceptable excipient” as used herein refers to a non-toxic material that may be formulated with a compound disclosed herein to provide a pharmaceutical composition. Preferably, the pharmaceutically acceptable excipient is inert and does not interfere with the pharmacological activity of a compound which it is formulated with. Pharmaceutically acceptable excipients useful in the manufacture of the pharmaceutical compositions disclosed herein are any of those well known in the art, and include without limitation, diluents, dispersing agents, granulating agents, surface active agents, emulsifiers, disintegrating agents, binding agents, preservatives, buffering agents, lubricating agents, ion exchangers, salts, electrolytes, waxes, and/or oils. For example, a pharmaceutically acceptable excipient may be alumina, aluminum stearate, lecithin, a serum protein (e.g., human serum albumin), a phosphate, glycine, sorbic acid, potassium sorbate, a glyceride mixture (e.g., saturated vegetable fatty acids), water, protamine sulfate, disodium hydrogen phosphate, potassium hydrogen phosphate, sodium chloride, a zinc salt, colloidal silica, magnesium trisilicate, polyvinyl pyrrolidone, cellulose or a derivative thereof, polyethylene glycol or a derivative thereof (e.g., PEG-300), sodium carboxymethylcellulose, a polyacrylate, a polyethylene-polyoxypropylene-block polymer, wool fat, a cyclodextrin (e.g., CAPTISOL®), dimethylacetamide (DMA), a polysorbate (e.g., a TWEEN®, e.g., TWEEN-20®), or a combination thereof.

The term “subject” as used herein refers to any animal, such as any mammal, including but not limited to, humans, non-human primates, rodents, and the like. Non-human primates include chimpanzees, cynomolgus monkeys, spider monkeys, and macaques (e.g., Rhesus). Rodents include mice, rats, woodchucks, ferrets, rabbits, and hamsters. Domestic and game animals include cows, horses, pigs, deer, bison, buffalo, feline species (e.g., domestic cat), canine species (e.g., dog, fox, wolf), avian species, and fish. In some embodiments, the subject is a mammal (e.g., a human, a rat, or a mouse). The subject can be male or female. The subject may be of any age, including an elderly human subject (e.g., 65 years or older), a human subject that is not elderly (e.g., less than 65 years old), or a human pediatric subject (e.g., 18 years old or less). In some embodiments, the subject is a human.

As used herein, the terms “treat,” “treatment,” “treating,” or grammatically related terms, refer to a method of reducing the effects of a disease or disorder. As is readily appreciated in the art, full eradication of the disease, disorder, or symptoms thereof is preferred but not a requirement for treatment. Desirable effects of treatment include, but are not limited to, preventing occurrence or recurrence of the disease or disorder, alleviation of symptoms, diminishment of any direct or indirect pathological consequences of the disease or disorder, or other improvement of any sign, symptom, or consequence of the disease or disorder, such as prolonged survival, less morbidity, and/or a lessening of side effects.

Throughout this disclosure, various embodiments can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 5, from 1 to 4, from 1 to 3, from 2 to 6, from 2 to 4, from 3 to 6, etc., as well as individual numbers within that range, e.g., 1, 2, 2.8, 3, 3.6, 4, 5, 5.4, and 6. As another example, a range such as 95-99% includes 95%, 96%, 97%, 98%, or 99% and all subranges such as 96-99%, 96-98%, 96-97%, 97-99%, 97-98%, etc. This applies regardless of the breadth of the range.

Various embodiments of the compounds, compositions, kits, and methods herein are described in further detail below, and additional definitions may be provided throughout the specification.

### Compounds

The compounds disclosed herein may comprise a structure of Formula (I):

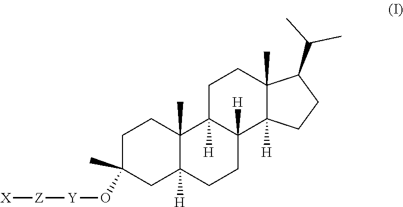

or a pharmaceutically acceptable salt, solvate, hydrate, tautomer, or stereoisomer thereof, wherein:
Y is —C(O)— or —C(R^{A})(R^{B})— (e.g., —CH₂—);
Z is absent, alkylene, alkenylene, alkynylene, heteroalkylene, cycloalkylene, heterocyclylene, arylene, heteroarylene, —NR¹—, —O—, or —S—, wherein the alkylene, alkenylene, alkynylene, or heteroalkylene is optionally substituted;
X is alkyl, alkenyl, alkynyl, heteroalkyl, cycloalkyl, heterocyclyl, aryl, heteroaryl, haloalkyl, alkylene-cycloalkyl, alkylene-heterocyclyl, alkylene-aryl, alkylene-heteroaryl, heteroalkylene-cycloalkyl, heteroalkylene-heterocyclyl, heteroalkylene-aryl, heteroalkylene-heteroaryl, cycloalkylene-alkyl, cycloalkylene-heteroalkyl, heterocyclylene-alkyl, heterocyclylene-heteroalkyl, —OC(O)-alkyl, —C(O)O-alkyl, —OC(O)-heteroalkyl, —C(O)O— heteroalkyl, —N₃, —NR¹R², —OR³, or —SR⁴, wherein the alkyl, alkylene, alkenyl, alkynyl, heteroalkyl, heteroalkylene, cycloalkyl, cycloalkylene, heterocyclyl, heterocyclylene, aryl, or heteroaryl is optionally substituted;
R¹ and R² are each independently hydrogen, alkyl, alkenyl, alkynyl, heteroalkyl, cycloalkyl, heterocyclyl, aryl, heteroaryl, alkylene-cycloalkyl, alkylene-heterocyclyl, alkylene-aryl, alkylene-heteroaryl, heteroalkylene-cycloalkyl, heteroalkylene-heterocyclyl, heteroalkylene-aryl, heteroalkylene-heteroaryl, cycloalkylene-alkyl, cycloalkylene-heteroalkyl, heterocyclylene-alkyl, or heterocyclylene-heteroalkyl, wherein the alkyl, alkylene, alkenyl, alkynyl, heteroalkyl, heteroalkylene, cycloalkyl, cycloalkylene, heterocyclyl, heterocyclylene, aryl, or heteroaryl is optionally substituted;
or R¹ and R², together with the atoms to which they are attached, form a 3-7 membered cycloalkyl, heterocyclyl, aryl, or heteroaryl, wherein the cycloalkyl, heterocyclyl, aryl, or heteroaryl is optionally substituted;
R³ is hydrogen, alkyl, alkenyl, alkynyl, heteroalkyl, cycloalkyl, heterocyclyl, aryl, heteroaryl, alkylene-cycloalkyl, alkylene-heterocyclyl, alkylene-aryl, alkylene-heteroaryl, heteroalkylene-cycloalkyl, heteroalkylene-heterocyclyl, heteroalkylene-aryl, heteroalkylene-heteroaryl, cycloalkylene-alkyl, cycloalkylene-heteroalkyl, heterocyclylene-alkyl, or heterocyclylene-heteroalkyl, wherein the alkyl, alkylene, alkenyl, alkynyl, heteroalkyl, heteroalkylene, cycloalkyl, cycloalkylene, heterocyclyl, heterocyclylene, aryl, or heteroaryl is optionally substituted;
R⁴ is hydrogen, alkyl, alkenyl, alkynyl, heteroalkyl, cycloalkyl, heterocyclyl, aryl, heteroaryl, alkylene-cycloalkyl, alkylene-heterocyclyl, alkylene-aryl, alkylene-heteroaryl, heteroalkylene-cycloalkyl, heteroalkylene-heterocyclyl, heteroalkylene-aryl, heteroalkylene-heteroaryl, wherein the alkyl, alkylene, alkenyl, alkynyl, heteroalkyl, heteroalkylene, cycloalkyl, cycloalkylene, heterocyclyl, heterocyclylene, aryl, or heteroaryl is optionally substituted; and
R^{A} and R^{B} are each independently hydrogen, alkyl, heteroalkyl, cycloalkyl, heterocyclyl, aryl, or heteroaryl, wherein the alkyl, heteroalkyl, cycloalkyl, heterocyclyl, aryl, or heteroaryl is optionally substituted.

Each optional substituent of a group in Formula (I) (e.g., an optional substituent on an alkyl, alkylene, alkenyl, alkenylene, alkynyl, alkynylene, heteroalkyl, heteroalkylene, cycloalkyl, cycloalkylene, heterocyclyl, heterocyclylene, aryl, or heteroaryl group) may be any suitable substituent, e.g., a substituent described herein. For example, the optional substituent can be alkyl (e.g., C₁-C₆ alkyl), alkenyl (e.g., C₂-C₆ alkenyl), alkynyl (e.g., C₂-C₆ alkynyl), heteroalkyl (e.g., C₁-C₆ heteroalkyl), haloalkyl (e.g., C₁-C₆ haloalkyl, e.g., —CH₂Cl, —CH₂I, —(CH₂)₂Cl, —(CH₂)₂I, or —CF₃), cycloalkyl (e.g., C₃-C₈ cycloalkyl, e.g., cyclopropyl, cyclobutyl, cyclopentyl, or cyclohexyl), heterocyclyl (e.g., C₃-C₈ heterocyclyl, e.g., morpholinyl, piperazinyl, N-methylpiperazinyl, azetidinyl, or N-Boc-azetidinyl), alkylaryl (e.g., benzyl), aryl (e.g., phenyl), heteroaryl (e.g., pyrrolyl, imidazolyl, or indolyl), halogen (e.g., —F, —Cl, —Br, or —I), hydroxyl, alkylcarbonyloxy, arylcarbonyloxy, alkoxy (e.g., methoxy (—OMe), ethoxy (—OEt), or benzyloxy (—OBn)), alkoxycarbonyloxy, aryloxycarbonyloxy, carboxylate, alkylcarbonyl, arylcarbonyl, alkoxycarbonyl, amino (e.g., —NH₂, —NHMe, or —NMe₂), aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl, and ureido), amidino, imino, sulfhydryl, alkylthio, arylthio, thiocarboxylate, sulfate, sulfinyl, sulfonyl, alkylsulfinyl, sulfonato, sulfamoyl, sulfonamido, oxo, nitro, trifluoromethyl, cyano, azido, alkyl-cycloalkyl, alkyl-heterocyclyl, alkylheteroaryl, —C(O)OH, —C(O)O-alkyl (e.g., —C(O)O-tBu or —C(O)OMe), —C(O)O-heteroalkyl, —C(O)O-alkylaryl, —OC(O)O-alkyl, —OC(O)O-heteroalkyl, —OC(O)O— alkylaryl, —C(O)NH₂, —C(O)NH-alkyl, —C(O)NH-heteroalkyl, —C(O)NH-alkylaryl, —NHC(O)O— alkyl (e.g., —NHC(O)OtBu), —NHC(O)O-heteroalkyl, —NHC(O)O-alkylaryl (e.g., —NH-Cbz), —C(O)N(alkyl)₂, —C(O)N(alkyl)(heteroalkyl), —C(O)N(heteroalkyl)₂, —N(alkyl)C(O)O-alkyl (e.g., N(Me)C(O)OtBu), —N(alkyl)C(O)O-heteroalkyl, —N(alkyl)C(O)O-alkylaryl, —N(alkyl)C(O)N(alkyl)₂, —N(alkyl)C(O)N(alkyl)(heteroalkyl), —N(alkyl)C(O)N(heteroalkyl)₂, —N(alkyl)C(O)NH-alkylaryl, ═NH, or ═N-alkyl. Cyclic groups (e.g., cycloalkyl, heterocyclyl, aryl, and heteroaryl) can be substituted at one or more ring positions with any suitable substituent, such as one of the substituents listed above.

In some aspects, Y is —C(O)—, and X is —NR¹R², —OR³, or —SR⁴. For example, Y may be —C(O)— and X may be —NR¹R²; Y may be —C(O)— and X may be —OR³; or Y may be —C(O)— and X may be —SR⁴. In some aspects, Y is —C(O)—, and Z is optionally substituted alkylene. For example, Y may be —C(O)—, and Z may be unsubstituted methylene; or Y may be —C(O)—, and Z may be unsubstituted methylene. In some aspects, Y is —C(O)—; X is —NR¹R², —OR³, or —SR⁴; and Z is optionally substituted alkylene. For example Y may be —C(O)—, X may be —NR¹R², and Z may be unsubstituted methylene; Y may be —C(O)—, X may be —OR³, and Z may be unsubstituted methylene; Y may be —C(O)—, X may be —SR⁴, and Z may be unsubstituted methylene; Y may be —C(O)—, X may be —NR¹R², and Z may be substituted methylene; Y may be —C(O)—, X may be —OR³, and Z may be substituted methylene; Y may be —C(O)—, X may be —SR⁴, and Z may be substituted methylene.

In some embodiments, Z is methylene (e.g., unsubstituted methylene). In some embodiments, Z is

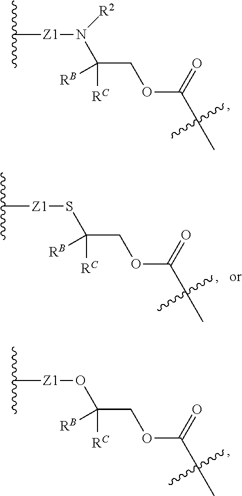

wherein Z1 is absent, alkylene (e.g., methylene, e.g., unsubstituted methylene), alkenylene, alkynylene, heteroalkylene, cycloalkylene, heterocyclylene, arylene, or heteroarylene, wherein the alkylene, alkenylene, alkynylene, or heteroalkylene is optionally substituted (e.g., with a substituent described herein); R² is hydrogen or alkyl; and R^{B} and R^{C} are each independently hydrogen, alkyl, heteroalkyl, cycloalkyl, heterocyclyl, aryl, or heteroaryl, wherein the alkyl, heteroalkyl, cycloalkyl, heterocyclyl, aryl, or heteroaryl is optionally substituted (e.g., with a substituent described herein); and each [symbol] denotes the point of attachment of Z to X or Y. In some embodiments, Z1 is absent.

In some embodiments, Z1 is optionally substituted alkylene (e.g., optionally substituted methylene), wherein the optional substituent is a substituent described herein. In some embodiments, Z is unsubstituted methylene. In some embodiments, Z1 is absent.

In some embodiments, Z is

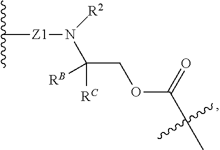

wherein R^{B}, R^{C}, and R² are each independently hydrogen, the nitrogen atom of —N(R²)— is attached to Y, the carbonyl group is attached to X, and Z1 is as defined herein (e.g., unsubstituted methylene). In some embodiments, Z is

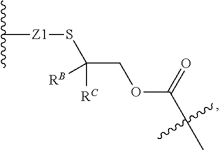

wherein R^{B} and R^{C} are each independently hydrogen, the sulfur atom of —S—C(R^{B})(R^{C})— is attached to Y, the carbonyl group is attached to X, and Z1 is as defined herein (e.g., unsubstituted methylene). In some embodiments, Z is

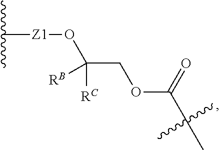

wherein R^{B} and R^{C} are each independently hydrogen, the oxygen atom of —O—C(R^{B})(R^{C})— is attached to Y, the carbonyl group is attached to X, and Z1 is as defined herein (e.g., unsubstituted methylene). In some embodiments, Z is

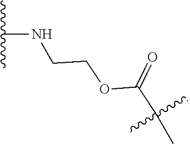 [SMILES: *NCCOC(*)=O]

In some embodiments, Z is

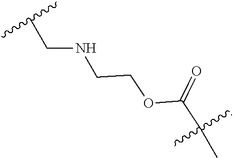 [SMILES: *CNCCOC(*)=O]

In some embodiments Z is

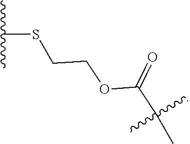 [SMILES: *SCCOC(*)=O]

In some embodiments, Z is

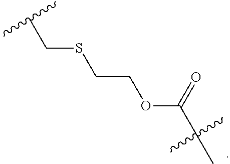 [SMILES: *CSCCOC(*)=O]

In some embodiments, Z is

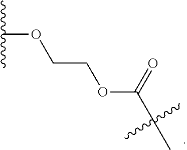 [SMILES: *OCCOC(*)=O]

In some embodiments, Z is

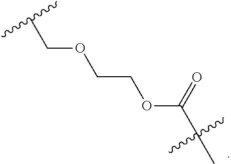 [SMILES: *COCCOC(*)=O]

In some embodiments, Z is absent.

In some embodiments, R¹ is hydrogen, and R² is hydrogen. In some embodiments, one of R¹ and R² is hydrogen, and the other of R¹ and R² is optionally substituted alkyl. In some embodiments, one of R¹ and R² is hydrogen, and the other of R¹ and R² is optionally substituted heteroalkyl. The alkyl or heteroalkyl groups may be substituted with any suitable substituent, e.g., a substituent described herein. For example, the alkyl or heteroalkyl groups may be substituted with one or more of an alkyl, oxo, or hydroxyl group.

Y may be —C(O)—, X may be —NR¹R², Z may be optionally substituted alkylene (e.g., optionally substituted methylene), and each of R¹ and R² may be independently hydrogen; Y may be —C(O)—, X may be —NR¹R², Z may be optionally substituted alkylene (e.g., optionally substituted methylene), R¹ may be hydrogen, and R² may be substituted alkyl (e.g., alkyl substituted with one or more alkyl, oxo, thiol, amino, hydroxyl, heterocyclyl, aryl, or heteroaryl groups); or Y may be —C(O)—, X may be —NR¹R², Z may be optionally substituted alkylene (e.g., optionally substituted methylene), R¹ may be hydrogen, and R² may be substituted heteroalkyl (e.g., heteroalkyl substituted with one or more alkyl, oxo, thiol, amino, hydroxyl, heterocyclyl, aryl, or heteroaryl groups). For example, Y may be —C(O)—, X may be —NR¹R², Z may be optionally substituted alkylene (e.g., optionally substituted methylene), R¹ may be hydrogen, and R² may be substituted tertiary-butyl (e.g., tertiary-butyl substituted with oxo and hydroxyl groups). For example, Y may be —C(O)—, X may be —NR¹R², Z may be unsubstituted methylene, R¹ may be hydrogen, and R² may be tertiary-butyl substituted with oxo and hydroxyl groups; or Y may be —C(O)—, X may be —NR¹R², Z may be substituted methylene, R¹ may be hydrogen, and R² may be tertiary-butyl substituted with oxo and hydroxyl groups.

Y may be —C(O)—, X may be —OR³, Z may be optionally substituted alkylene (e.g., optionally substituted methylene), and R³ may be hydrogen; Y may be —C(O)—, X may be —OR³, Z may be optionally substituted alkylene (e.g., optionally substituted methylene), and R³ may be optionally substituted alkyl (e.g., alkyl substituted with one or more alkyl, oxo, thiol, amino, hydroxyl, heterocyclyl, aryl, or heteroaryl groups); or Y may be —C(O)—, X may be —OR³, Z may be optionally substituted alkylene (e.g., optionally substituted methylene), and R³ may be optionally substituted heteroalkyl (e.g., heteroalkyl substituted with one or more alkyl, oxo, thiol, amino, hydroxyl, heterocyclyl, aryl, or heteroaryl groups). For example, Y may be —C(O)—, X may be —OR³, Z may be optionally substituted alkylene (e.g., optionally substituted methylene), and R³ may be substituted tertiary-butyl (e.g., tertiary-butyl substituted with oxo, thiol, hydroxyl, and/or amino groups). For example, Y may be —C(O)—, X may be —OR³, Z may be unsubstituted methylene, and R³ may be tertiary-butyl substituted with oxo, thiol, hydroxyl, and/or amino groups; or Y may be —C(O)—, X may be —OR³, Z may be substituted methylene, and R³ may be tertiary-butyl substituted with oxo, hydroxyl, thiol, and/or amino groups.

Y may be —C(O)—, X may be —SR⁴, Z may be optionally substituted alkylene (e.g., optionally substituted methylene), and R⁴ may be hydrogen; Y may be —C(O)—, X may be —SR⁴, Z may be optionally substituted alkylene (e.g., optionally substituted methylene), and R⁴ may be optionally substituted alkyl (e.g., alkyl substituted with one or more alkyl, oxo, thiol, amino, hydroxyl, heterocyclyl, aryl, or heteroaryl groups); or Y may be —C(O)—, X may be —SR⁴, Z may be optionally substituted alkylene (e.g., optionally substituted methylene), and R⁴ may be optionally substituted heteroalkyl (e.g., heteroalkyl substituted with one or more alkyl, oxo, thiol, amino, hydroxyl, heterocyclyl, aryl, or heteroaryl groups). For example, Y may be —C(O)—, X may be —SR⁴, Z may be optionally substituted alkylene (e.g., optionally substituted methylene), and R⁴ may be substituted tertiary-butyl (e.g., tertiary-butyl substituted with oxo, thiol, hydroxyl, and/or amino groups). For example, Y may be —C(O)—, X may be —SR⁴, Z may be unsubstituted methylene, and R⁴ may be tertiary-butyl substituted with oxo, thiol, hydroxyl, and/or amino groups; or Y may be —C(O)—, X may be —SR⁴, Z may be substituted methylene, and R⁴ may be tertiary-butyl substituted with oxo, hydroxyl, thiol, and/or amino groups.

The compound of Formula (I) may be a compound of Formula (I-I):

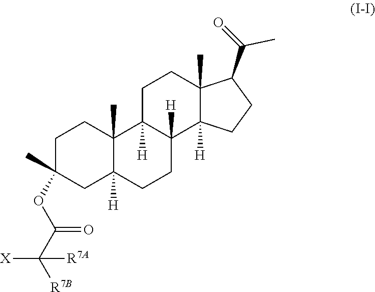

or a pharmaceutically acceptable salt, solvate, hydrate, tautomer, or stereoisomer thereof, wherein:
X is alkyl, alkenyl, alkynyl, heteroalkyl, cycloalkyl, heterocyclyl, aryl, heteroaryl, haloalkyl, alkylene-cycloalkyl, alkylene-heterocyclyl, alkylene-aryl, alkylene-heteroaryl, heteroalkylene-cycloalkyl, heteroalkylene-heterocyclyl, heteroalkylene-aryl, heteroalkylene-heteroaryl, cycloalkylene-alkyl, cycloalkylene-heteroalkyl, heterocyclylene-alkyl, heterocyclylene-heteroalkyl, —OC(O)-alkyl, —C(O)O-alkyl, —OC(O)-heteroalkyl, —C(O)O— heteroalkyl, —N₃, —NR¹R², —OR³, or —SR⁴, wherein the alkyl, alkylene, alkenyl, alkynyl, heteroalkyl, heteroalkylene, cycloalkyl, cycloalkylene, heterocyclyl, heterocyclylene, aryl, or heteroaryl is optionally substituted;
R¹ and R² are each independently hydrogen, alkyl, alkenyl, alkynyl, heteroalkyl, cycloalkyl, heterocyclyl, aryl, heteroaryl, alkylene-cycloalkyl, alkylene-heterocyclyl, alkylene-aryl, alkylene-heteroaryl, heteroalkylene-cycloalkyl, heteroalkylene-heterocyclyl, heteroalkylene-aryl, heteroalkylene-heteroaryl, cycloalkylene-alkyl, cycloalkylene-heteroalkyl, heterocyclylene-alkyl, or heterocyclylene-heteroalkyl, wherein the alkyl, alkylene, alkenyl, alkynyl, heteroalkyl, heteroalkylene, cycloalkyl, cycloalkylene, heterocyclyl, heterocyclylene, aryl, or heteroaryl is optionally substituted;
or R¹ and R², together with the atoms to which they are attached, form a 3-7 membered cycloalkyl, heterocyclyl, aryl, or heteroaryl, wherein the cycloalkyl, heterocyclyl, aryl, or heteroaryl is optionally substituted;
R³ is hydrogen, alkyl, alkenyl, alkynyl, heteroalkyl, cycloalkyl, heterocyclyl, aryl, heteroaryl, alkylene-cycloalkyl, alkylene-heterocyclyl, alkylene-aryl, alkylene-heteroaryl, heteroalkylene-cycloalkyl, heteroalkylene-heterocyclyl, heteroalkylene-aryl, heteroalkylene-heteroaryl, cycloalkylene-alkyl, cycloalkylene-heteroalkyl, heterocyclylene-alkyl, or heterocyclylene-heteroalkyl, wherein the alkyl, alkylene, alkenyl, alkynyl, heteroalkyl, heteroalkylene, cycloalkyl, cycloalkylene, heterocyclyl, heterocyclylene, aryl, or heteroaryl is optionally substituted; and
R^{7A} and R^{7B} are each independently hydrogen or optionally substituted alkyl.

Each optional substituent of a group in Formula (I-I) (e.g., an optional substituent on an alkyl, alkylene, alkenyl, alkenylene, alkynyl, alkynylene, heteroalkyl, heteroalkylene, cycloalkyl, cycloalkylene, heterocyclyl, heterocyclylene, aryl, or heteroaryl group) may be any suitable substituent, e.g., a substituent described herein. For example, the optional substituent can be alkyl (e.g., C₁-C₆ alkyl), alkenyl (e.g., C₂-C₆ alkenyl), alkynyl (e.g., C₂-C₆ alkynyl), heteroalkyl (e.g., C₁-C₆ heteroalkyl), haloalkyl (e.g., C₁-C₆ haloalkyl, e.g., —CH₂Cl, —CH₂I, —(CH₂)₂Cl, —(CH₂)₂I, or —CF₃), cycloalkyl (e.g., C₃-C₈ cycloalkyl, e.g., cyclopropyl, cyclobutyl, cyclopentyl, or cyclohexyl), heterocyclyl (e.g., C₃-C₈ heterocyclyl, e.g., morpholinyl, piperazinyl, N-methylpiperazinyl, azetidinyl, or N-Boc-azetidinyl), alkylaryl (e.g., benzyl), aryl (e.g., phenyl), heteroaryl (e.g., pyrrolyl, imidazolyl, or indolyl), halogen (e.g., —F, —Cl, —Br, or —I), hydroxyl, alkylcarbonyloxy, arylcarbonyloxy, alkoxy (e.g., —OMe, —OEt, or —OBn), alkoxycarbonyloxy, aryloxycarbonyloxy, carboxylate, alkylcarbonyl, arylcarbonyl, alkoxycarbonyl, amino (e.g., —NH₂, —NHMe, or —NMe₂), aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl, and ureido), amidino, imino, sulfhydryl, alkylthio, arylthio, thiocarboxylate, sulfate, sulfinyl, sulfonyl, alkylsulfinyl, sulfonato, sulfamoyl, sulfonamido, oxo, nitro, trifluoromethyl, cyano, azido, alkyl-cycloalkyl, alkyl-heterocyclyl, alkylheteroaryl, —C(O)OH, —C(O)O-alkyl (e.g., —C(O)O-tBu or —C(O)OMe), —C(O)O— heteroalkyl, —C(O)O-alkylaryl, —OC(O)O-alkyl, —OC(O)O-heteroalkyl, —OC(O)O-alkylaryl, —C(O)NH₂, —C(O)NH-alkyl, —C(O)NH-heteroalkyl, —C(O)NH-alkylaryl, —NHC(O)O-alkyl (e.g., —NHC(O)OtBu), —NHC(O)O-heteroalkyl, —NHC(O)O-alkylaryl (e.g., —NH-Cbz), —C(O)N(alkyl)₂, —C(O)N(alkyl)(heteroalkyl), —C(O)N(heteroalkyl)₂, —N(alkyl)C(O)O-alkyl (e.g., N(Me)C(O)OtBu), —N(alkyl)C(O)O-heteroalkyl, —N(alkyl)C(O)O-alkylaryl, —N(alkyl)C(O)N(alkyl)₂, —N(alkyl)C(O)N(alkyl)(heteroalkyl), —N(alkyl)C(O)N(heteroalkyl)₂, —N(alkyl)C(O)NH-alkylaryl, ═NH, or ═N-alkyl. Cyclic groups (e.g., cycloalkyl, heterocyclyl, aryl, and heteroaryl) can be substituted at one or more ring positions with any suitable substituent, such as one of the substituents listed above.

In a compound disclosed herein, e.g., a compound of Formula (I) or Formula (I-I), X may be a moiety selected from the group consisting of

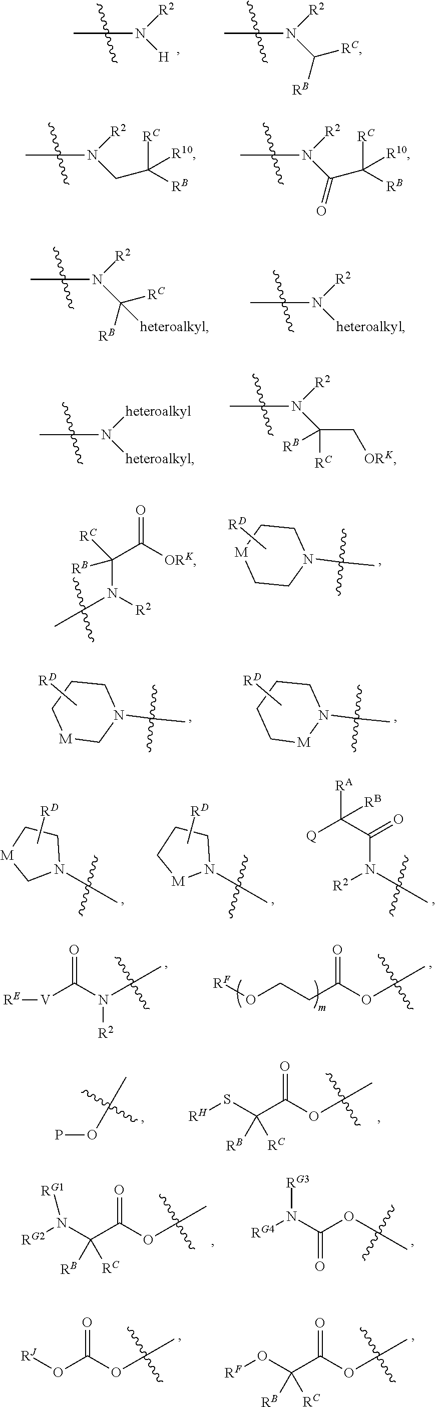

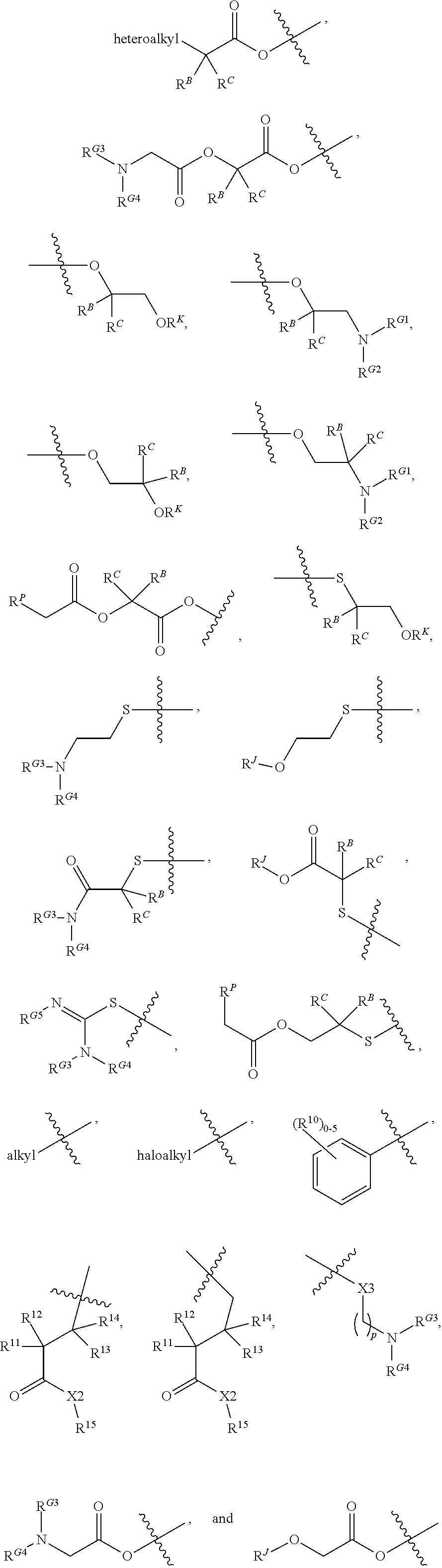

wherein
R² is hydrogen or alkyl;
M is —C(H)(R^{D})— (e.g., —CH₂—), —O—, or —N(R^{D})— (e.g., —N(Me)—);
P is an amino acid residue (e.g., glycine, alanine, valine, leucine, or proline), that may be a D-amino acid residue or L-amino acid residue, and is optionally substituted (e.g., at a terminal nitrogen atom, e.g., by a carbonate group, e.g., a carboxybenzyl (Cbz) group),
or P is a dipeptide moiety (e.g., a leucine-leucine moiety) that may comprise D-amino acids, L-amino acids, or a combination thereof, and is optionally substituted (e.g., at one or more nitrogen atoms, e.g., at a terminal nitrogen atom, e.g., by a carbonate group, e.g., a carboxybenzyl (Cbz) group);
Q is heteroalkyl or heterocyclyl, wherein the heteroalkyl or heterocyclyl is optionally substituted;
V is —O—, —N(H)—, or —N(Me)—;
X2 and X3 are each independently —CH₂—, —O—, —S—, or —N(R^{G3})— (e.g., —NH—);
R^{A}, R^{B}, R^{C}, R^{D}, R^{E}, and R^{K} are each independently hydrogen, alkyl, heteroalkyl, cycloalkyl, heterocyclyl, aryl, or heteroaryl, wherein the alkyl, heteroalkyl, cycloalkyl, heterocyclyl, aryl, or heteroaryl is optionally substituted;
R^{F} and R^{H} are each independently hydrogen or alkyl;
R^{G1} and R^{G2} are each independently hydrogen, alkyl, or an amino acid residue (e.g., glycine, alanine, valine, leucine or proline), that may be a D-amino acid residue or L-amino acid residue, and is optionally substituted (e.g., at a terminal nitrogen atom, e.g., by a carbonate group, e.g., a carboxybenzyl (Cbz) group),
or R^{G1} and R^{G2}, together with the atoms to which they are attached, form a 3-7 membered cycloalkyl, heterocyclyl, aryl, or heteroaryl, wherein the cycloalkyl, heterocyclyl, aryl, or heteroaryl is optionally substituted;
R^{G3} and R^{G4} are each independently hydrogen, alkyl, or heteroalkyl, wherein the alkyl or heteroalkyl is optionally substituted;
or R^{G3} and R^{G4}, together with the atoms to which they are attached, form a 3-7 membered cycloalkyl, heterocyclyl, aryl, or heteroaryl, wherein the cycloalkyl, heterocyclyl, aryl, or heteroaryl is optionally substituted;
R^{G5} is hydrogen, alkyl, or heteroalkyl, wherein the alkyl or heteroalkyl is optionally substituted;
R^{J} is hydrogen, alkyl, alkylene-heterocyclyl, heteroalkyl, or heterocyclyl, wherein the alkyl, alkylene, heteroalkyl, or heterocyclyl is optionally substituted;
R^{P} is halo (e.g., chloro), heterocyclyl (e.g., morpholinyl), —NH-alkyl, —NH-heteroalkyl, —N-(alkyl)₂ (e.g., —N(Me)₂), or —N-(heteroalkyl)₂ (e.g., —N(CH₂CH₂OH)₂);
R¹⁰ is alkyl, alkenyl, alkynyl, haloalkyl, halo, —OR, —N(R^{G3})(R^{G4}); —NO₂, —CN, or —NC;
R¹¹, R¹², R¹³, and R¹⁴ are each independently hydrogen, alkyl, alkenyl, alkynyl, heteroalkyl, haloalkyl, halo, or —N(R^{G3})(R^{G4}), wherein the alkyl, alkenyl, alkynyl, heteroalkyl, or haloalkyl is optionally substituted,
or one of R¹¹ and R¹², and one of R¹³ and R¹⁴, together with the atoms to which they are attached, form a 3-7 membered cycloalkyl, heterocyclyl, aryl, or heteroaryl, wherein the cycloalkyl, heterocyclyl, aryl, or heteroaryl is optionally substituted;
R¹⁵ is hydrogen, alkyl, alkenyl, alkynyl, heteroalkyl, haloalkyl, or halo; wherein the alkyl, alkenyl, alkynyl, heteroalkyl, or haloalkyl is optionally substituted;
m is an integer of 1-5;
p is an integer between 0 and 4; and
[symbol] denotes the point of attachment of X to another group of the compound, e.g., an attachment of X to Z, or X to Y when Z is absent in Formula (I), or an attachment of X to the carbon atom of the —C(R^{7A})(R^{7B}) group in Formula (I-I).

Each optional substituent of a group described above (e.g., an optional substituent on an alkyl, alkylene, alkenyl, alkenylene, alkynyl, alkynylene, heteroalkyl, heteroalkylene, cycloalkyl, cycloalkylene, heterocyclyl, heterocyclylene, aryl, or heteroaryl group) may be any suitable substituent, e.g., a substituent described herein. For example, the optional substituent can be alkyl (e.g., C₁-C₆ alkyl), alkenyl (e.g., C₂-C₆ alkenyl), alkynyl (e.g., C₂-C₆ alkynyl), heteroalkyl (e.g., C₁-C₆ heteroalkyl), haloalkyl (e.g., C₁-C₆ haloalkyl, e.g., —CH₂Cl, —CH₂I, —(CH₂)₂Cl, —(CH₂)₂I, or —CF₃), cycloalkyl (e.g., C₃-C₈ cycloalkyl, e.g., cyclopropyl, cyclobutyl, cyclopentyl, or cyclohexyl), heterocyclyl (e.g., C₃-C₈ heterocyclyl, e.g., morpholinyl, piperazinyl, N-methylpiperazinyl, azetidinyl, or N-Boc-azetidinyl), alkylaryl (e.g., benzyl), aryl (e.g., phenyl), heteroaryl (e.g., pyrrolyl, imidazolyl, or indolyl), halogen (e.g., —F, —Cl, —Br, or —I), hydroxyl, alkylcarbonyloxy, arylcarbonyloxy, alkoxy (e.g., —OMe, —OEt, or —OBn), alkoxycarbonyloxy, aryloxycarbonyloxy, carboxylate, alkylcarbonyl, arylcarbonyl, alkoxycarbonyl, amino (e.g., —NH₂, —NHMe, or —NMe₂), aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl, and ureido), amidino, imino, sulfhydryl, alkylthio, arylthio, thiocarboxylate, sulfate, sulfinyl, sulfonyl, alkylsulfinyl, sulfonato, sulfamoyl, sulfonamido, oxo, nitro, trifluoromethyl, cyano, azido, alkyl-cycloalkyl, alkyl-heterocyclyl, alkylheteroaryl, —C(O)OH, —C(O)O-alkyl (e.g., —C(O)O-tBu or —C(O)OMe), —C(O)O— heteroalkyl, —C(O)O-alkylaryl, —OC(O)O-alkyl, —OC(O)O-heteroalkyl, —OC(O)O-alkylaryl, —C(O)NH₂, —C(O)NH-alkyl, —C(O)NH-heteroalkyl, —C(O)NH-alkylaryl, —NHC(O)O-alkyl (e.g., —NHC(O)OtBu), —NHC(O)O-heteroalkyl, —NHC(O)O-alkylaryl (e.g., —NH-Cbz), —C(O)N(alkyl)₂, —C(O)N(alkyl)(heteroalkyl), —C(O)N(heteroalkyl)₂, —N(alkyl)C(O)O-alkyl (e.g., N(Me)C(O)OtBu), —N(alkyl)C(O)O-heteroalkyl, —N(alkyl)C(O)O-alkylaryl, —N(alkyl)C(O)N(alkyl)₂, —N(alkyl)C(O)N(alkyl)(heteroalkyl), —N(alkyl)C(O)N(heteroalkyl)₂, —N(alkyl)C(O)NH-alkylaryl, ═NH, or ═N-alkyl. Cyclic groups (e.g., cycloalkyl, heterocyclyl, aryl, and heteroaryl) can be substituted at one or more ring positions with any suitable substituent, such as one of the substituents listed above.

When P is an amino acid residue (e.g., glycine, alanine, valine, leucine, or proline) or dipeptide moiety (e.g., a leucine-leucine moiety), it may comprise one or more D-amino acids, L-amino acids, or a combination thereof. The amino acid residue or dipeptide moiety may be attached via its C-terminus, and may further comprise a free N-terminus or an N-terminus that is optionally substituted, e.g., with a carboxybenzyl (Cbz) group). The amino acid residue or dipeptide moiety may alternatively be attached via its N-terminus, and may further comprise a free C-terminus (e.g., —C(O)OH), a C-terminus that is optionally substituted.

In some embodiments, X is

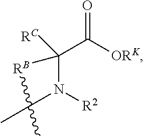

R² is H, R^{B} and R^{C} are each independently alkyl (e.g., methyl), and R^{K} is hydrogen.

In some embodiments, Y is —C(O)— and Z is alkylene (e.g., methylene), and X is

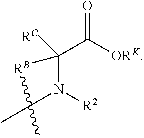

In some embodiments, when X is —NR¹R², R¹ and R² are not both hydrogen. In some embodiments, when X is —NR¹R², and R¹ is hydrogen, R² is not unsubstituted alkyl (e.g., methyl, ethyl, propyl, butyl, pentyl, hexyl, heptyl, octyl, nonyl, decyl, undecyl, or dodecyl). In some embodiments, when X is —NR¹R², and R¹ is alkyl (e.g., methyl), R² is not unsubstituted alkyl (e.g., methyl, ethyl, propyl, butyl, pentyl, hexyl, heptyl, octyl, nonyl, decyl, undecyl, or dodecyl).

In some embodiments, when X is —OR³, R³ is not hydrogen. In some embodiments, when X is —OR³, R³ is not unsubstituted alkyl (e.g., methyl, ethyl, propyl, butyl, pentyl, hexyl, heptyl, octyl, nonyl, decyl, undecyl, or dodecyl).

In some embodiments, when X is —SR⁴, R⁴ is not hydrogen. In some embodiments, when X is —SR⁴, R⁴ is not unsubstituted alkyl (e.g., methyl, ethyl, propyl, butyl, pentyl, hexyl, heptyl, octyl, nonyl, decyl, undecyl, or dodecyl).

In a compound disclosed herein, e.g. a compound of Formula (I) or Formula (I-I), X may be a moiety selected from the group consisting of

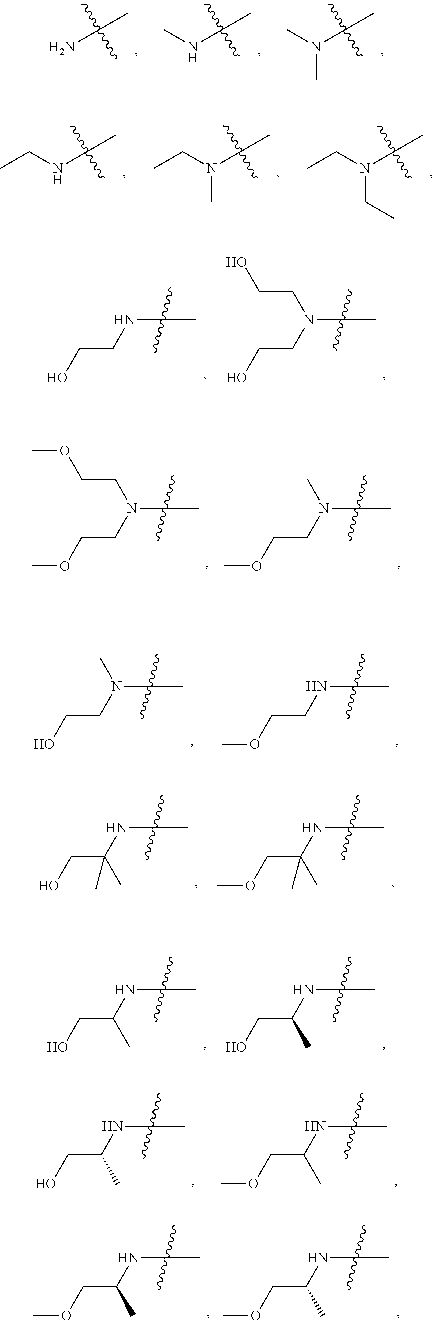

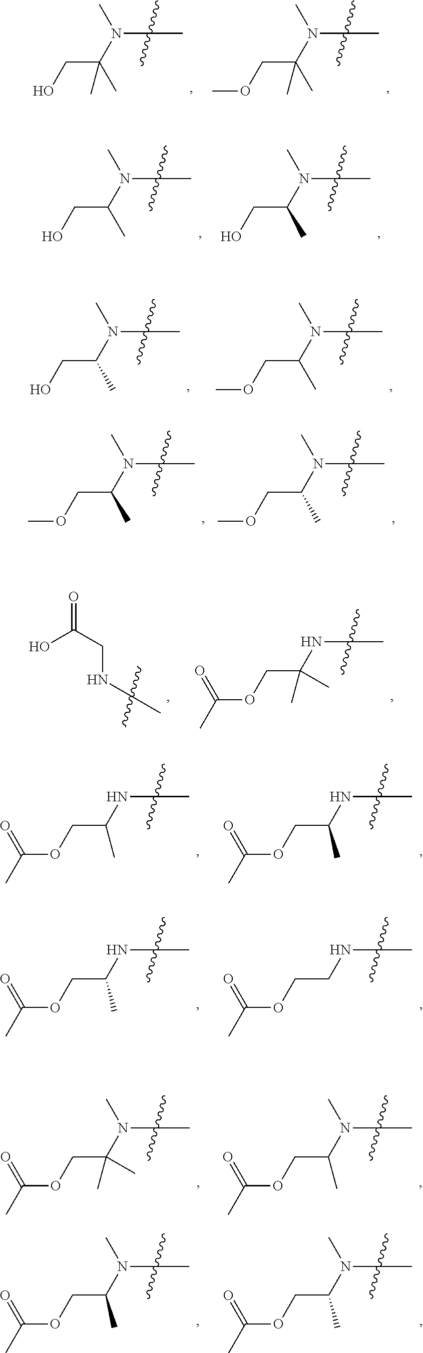

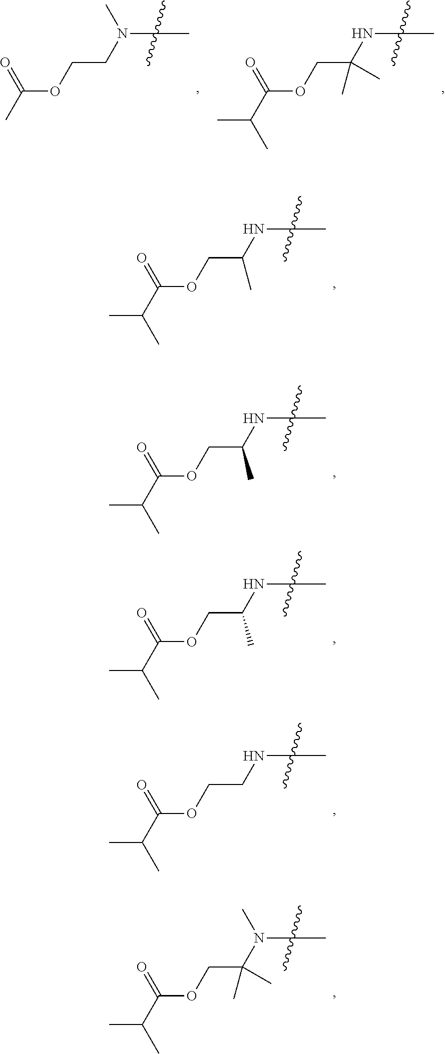

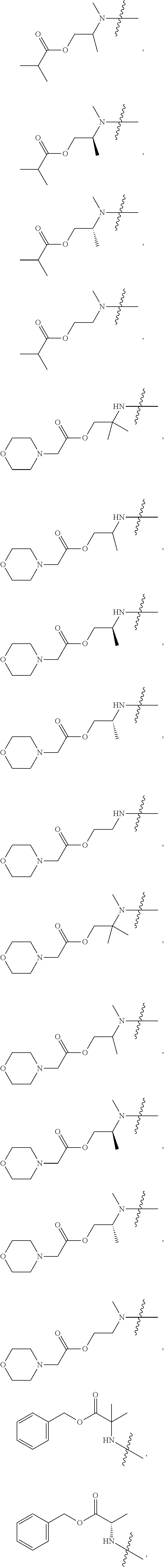

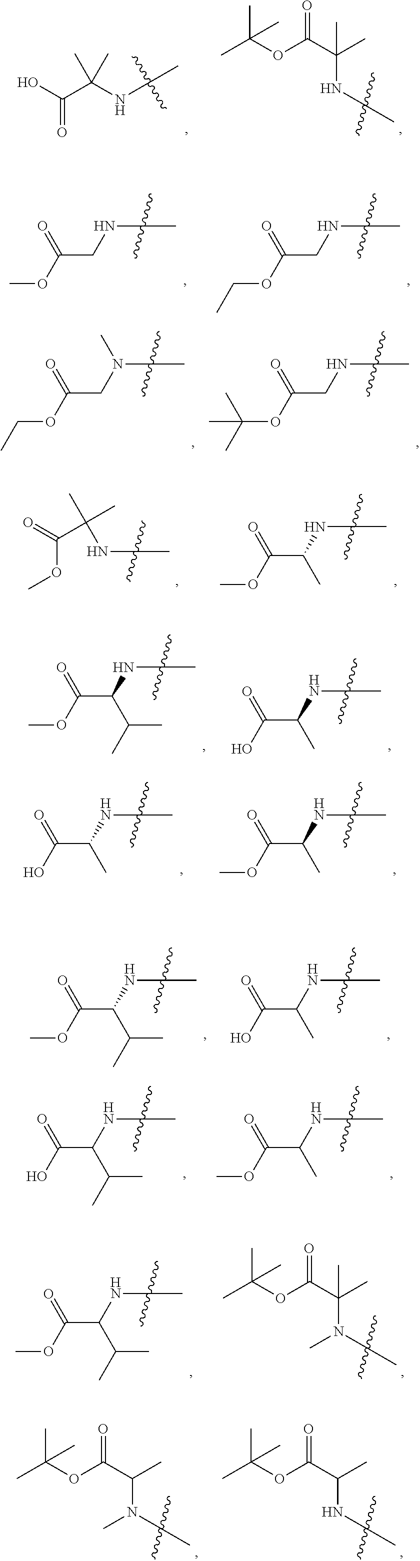

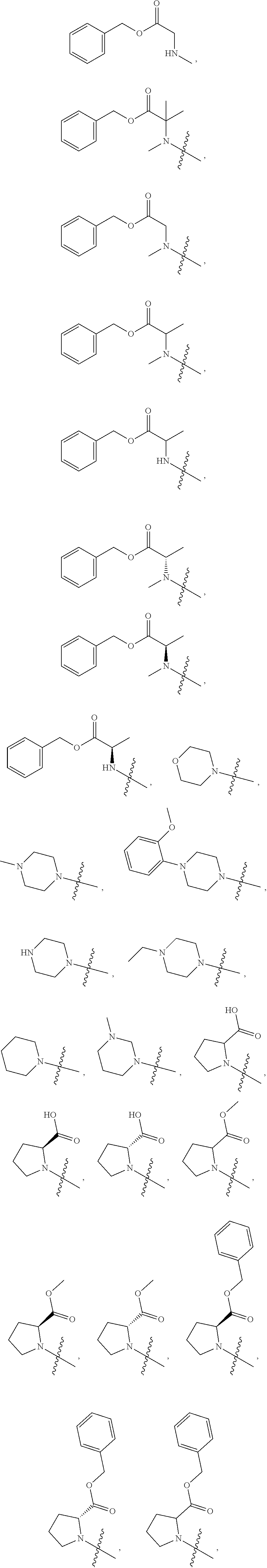

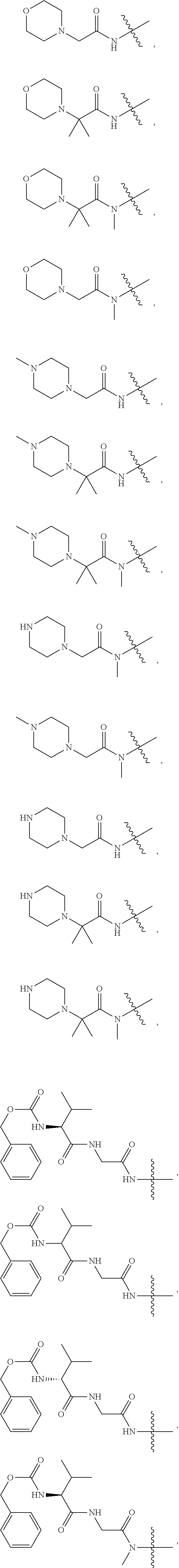

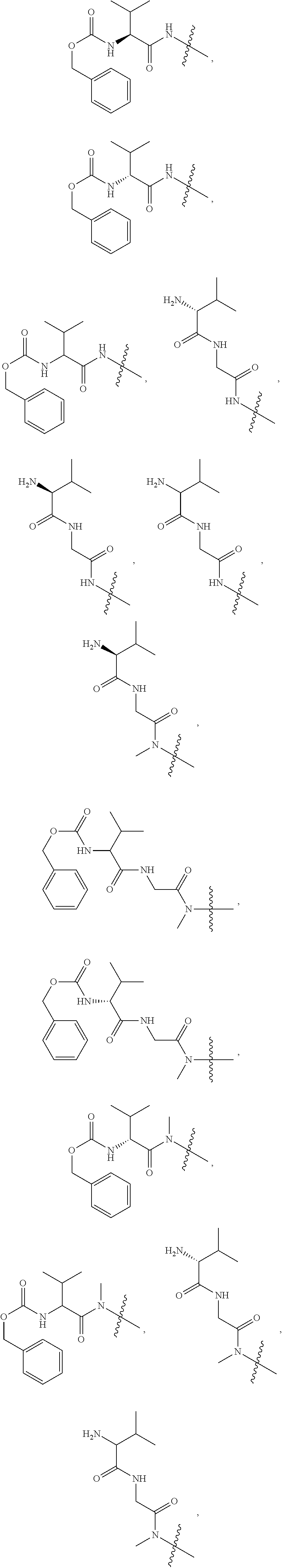

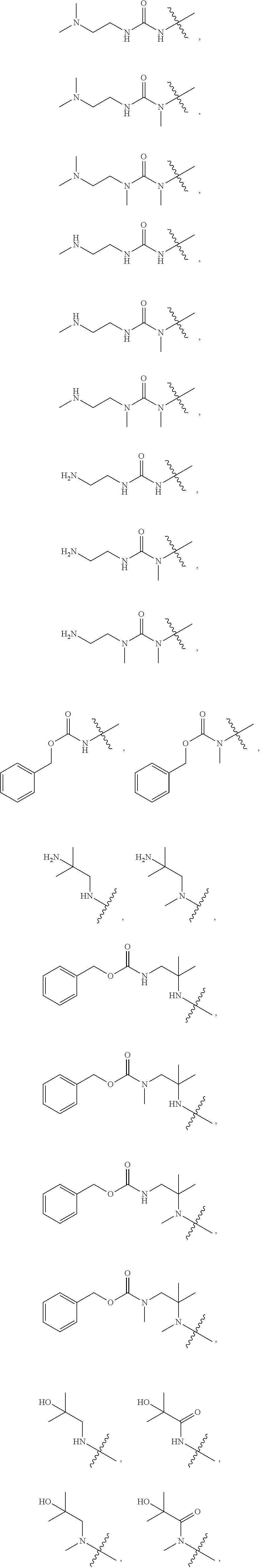

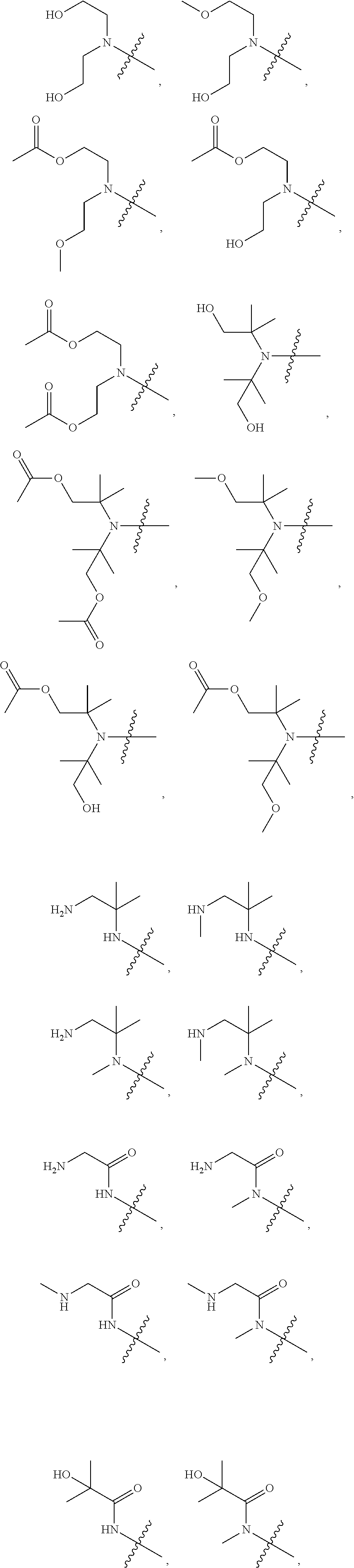

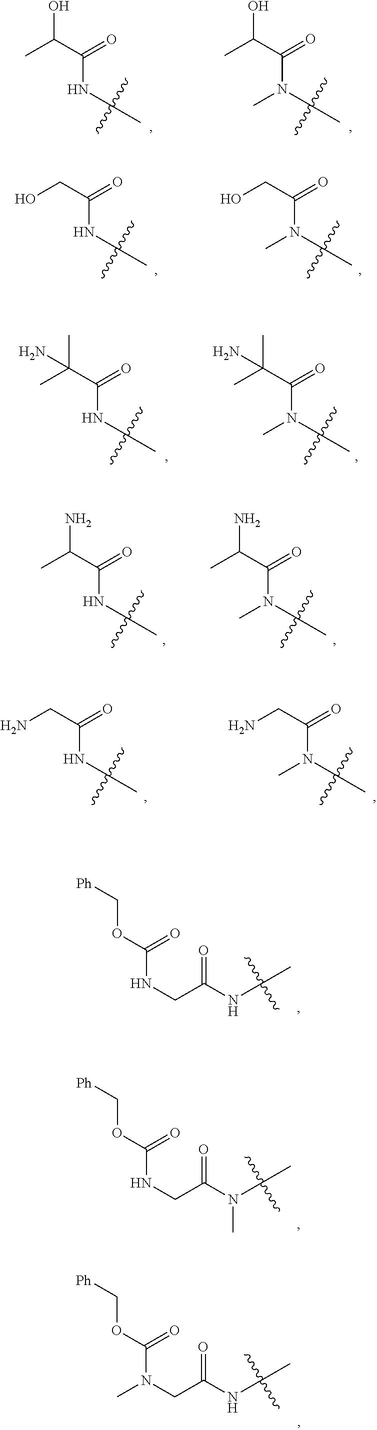

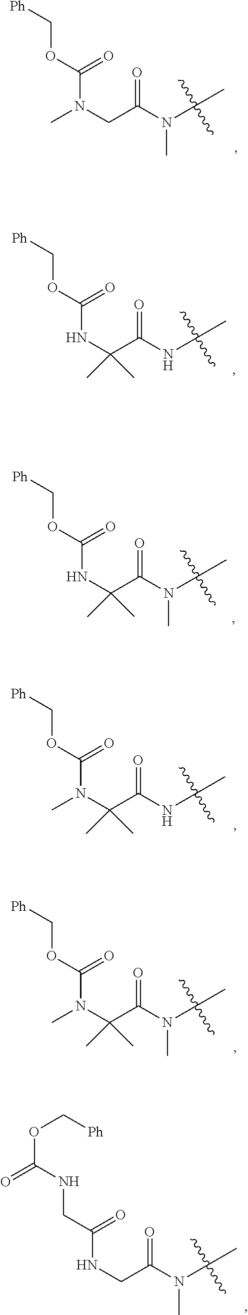

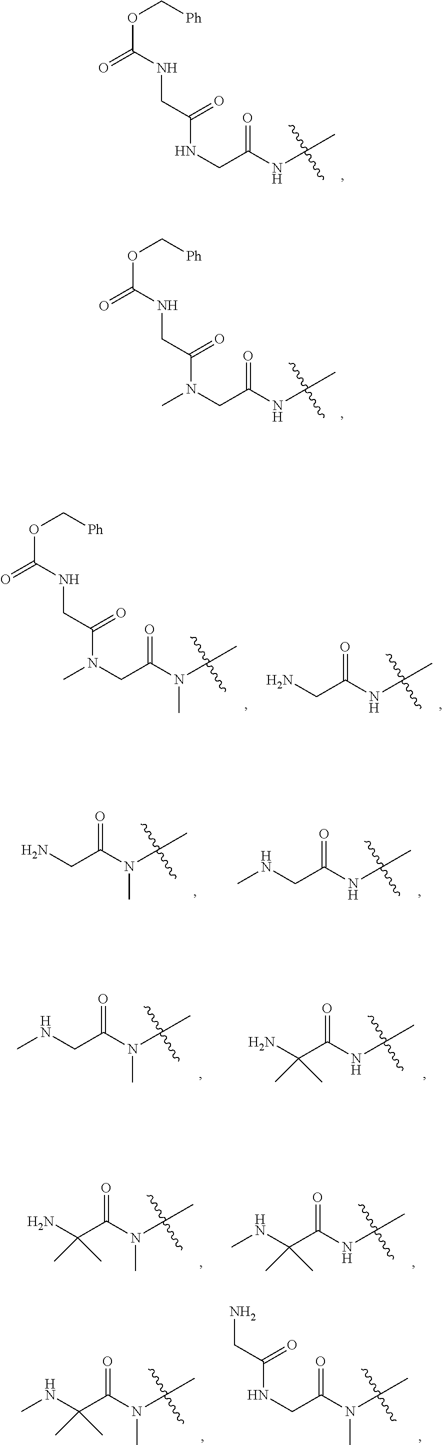

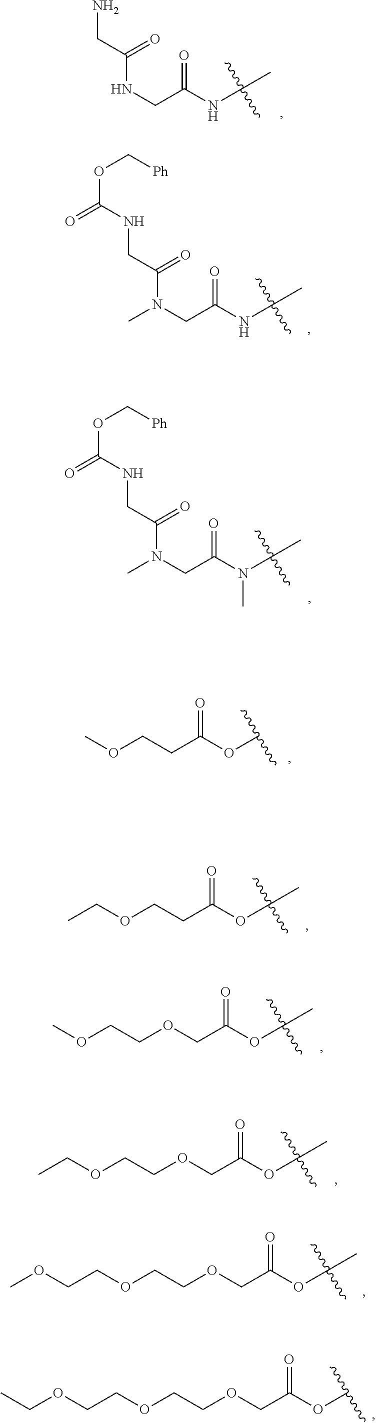

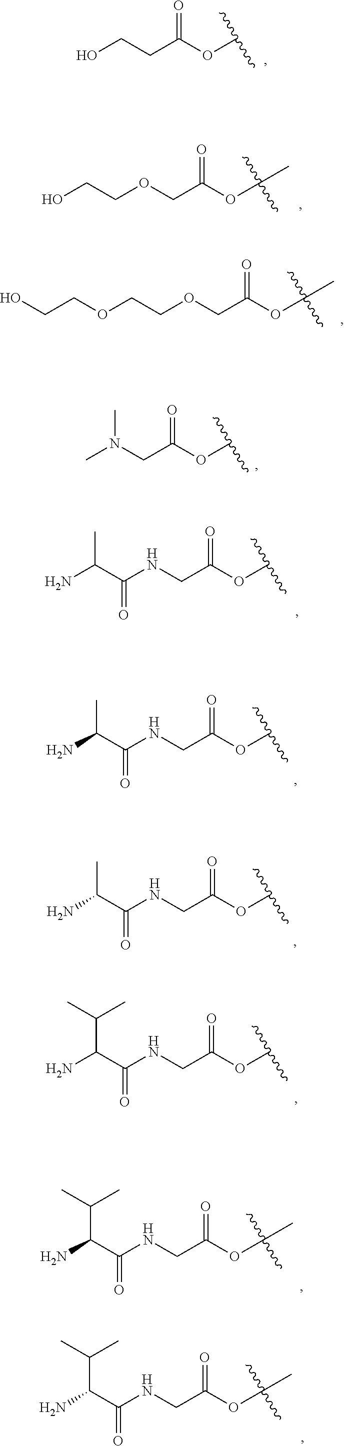

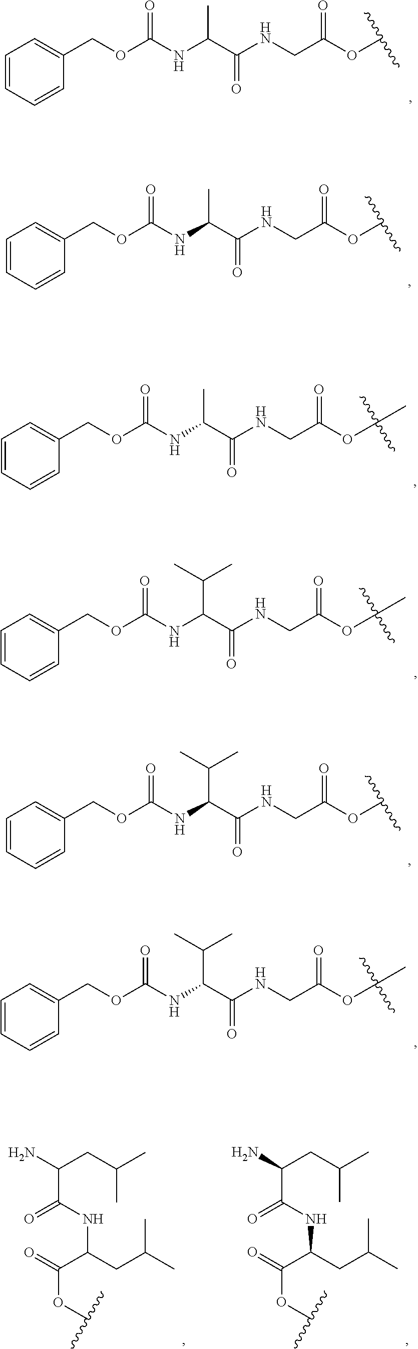

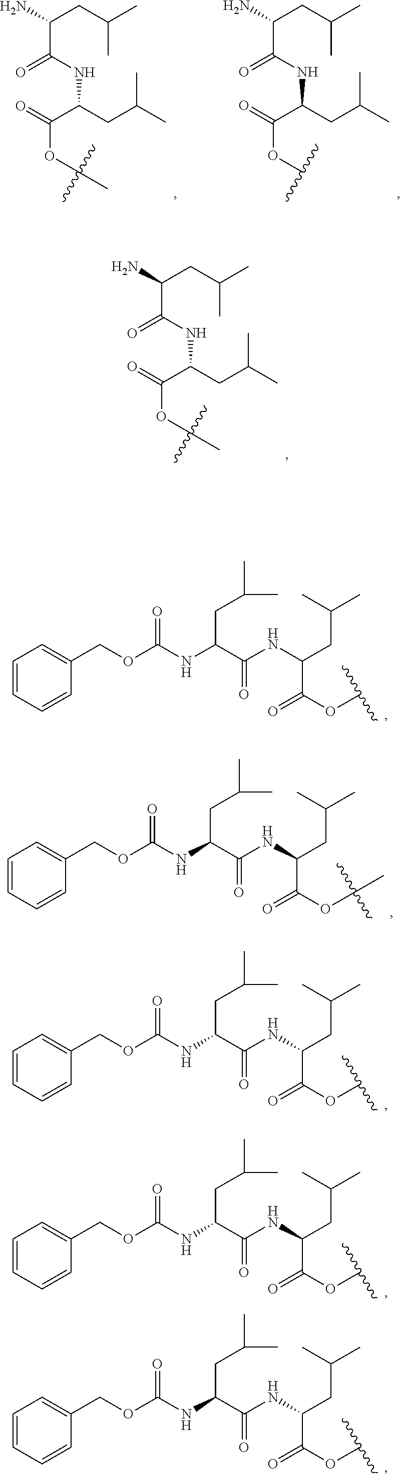

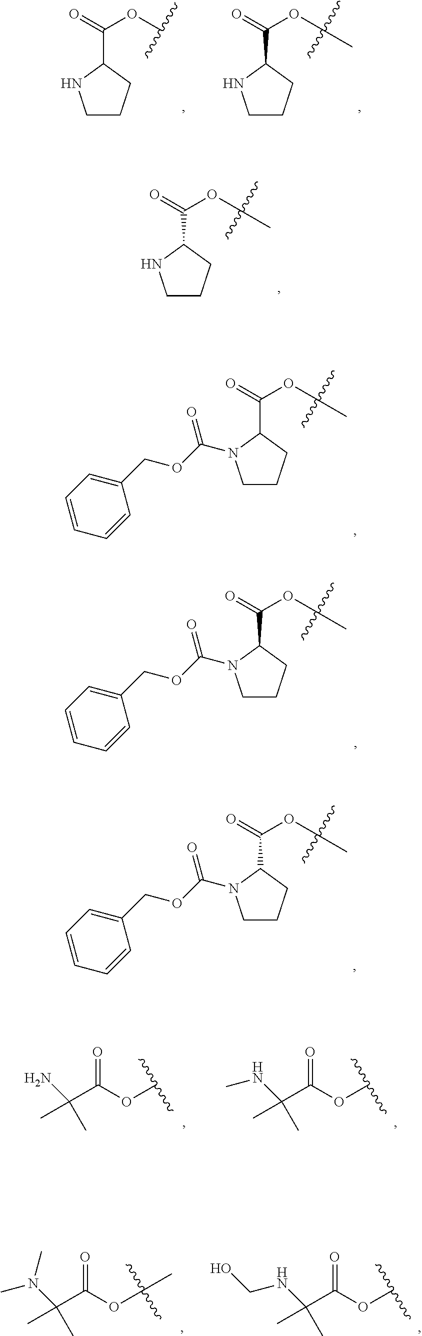

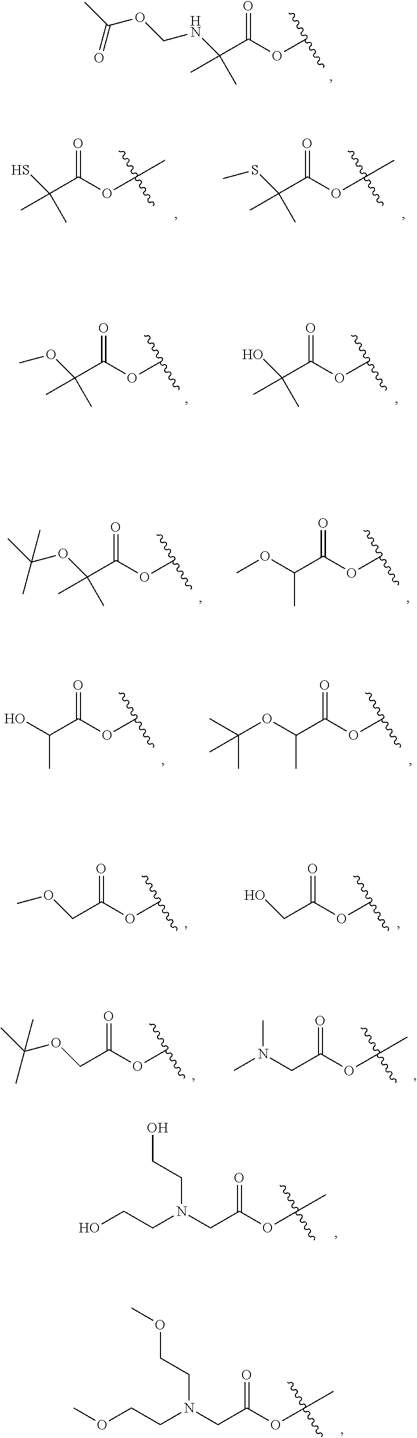

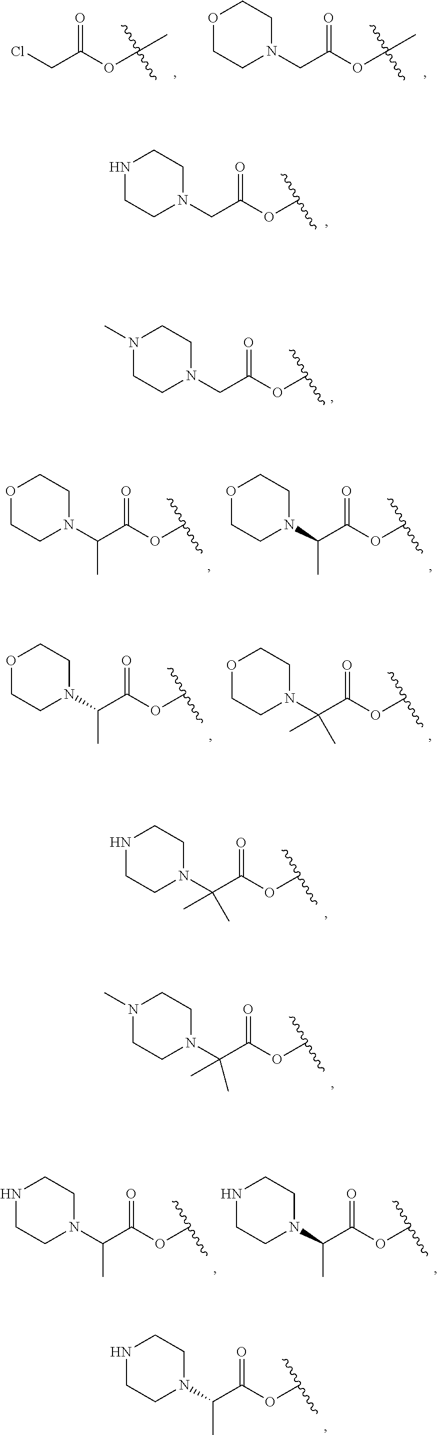

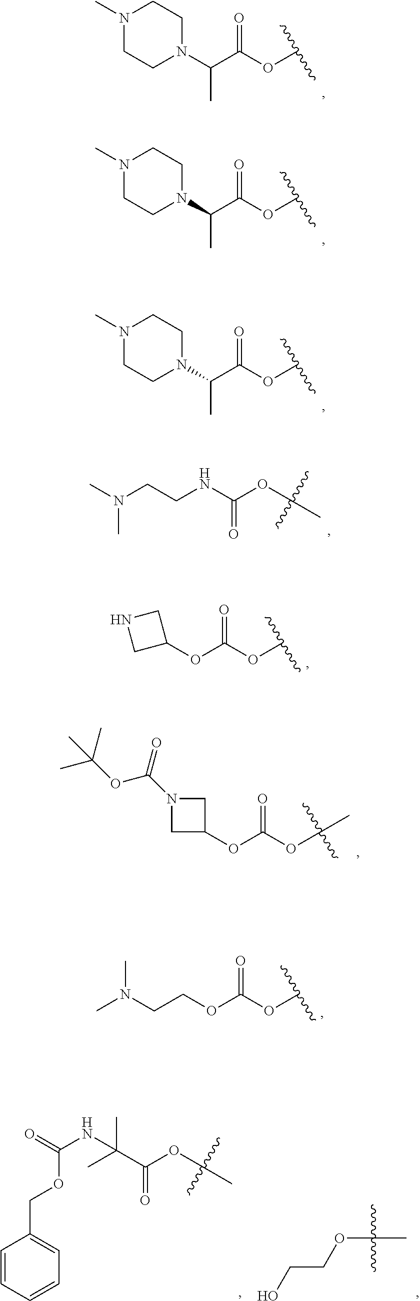

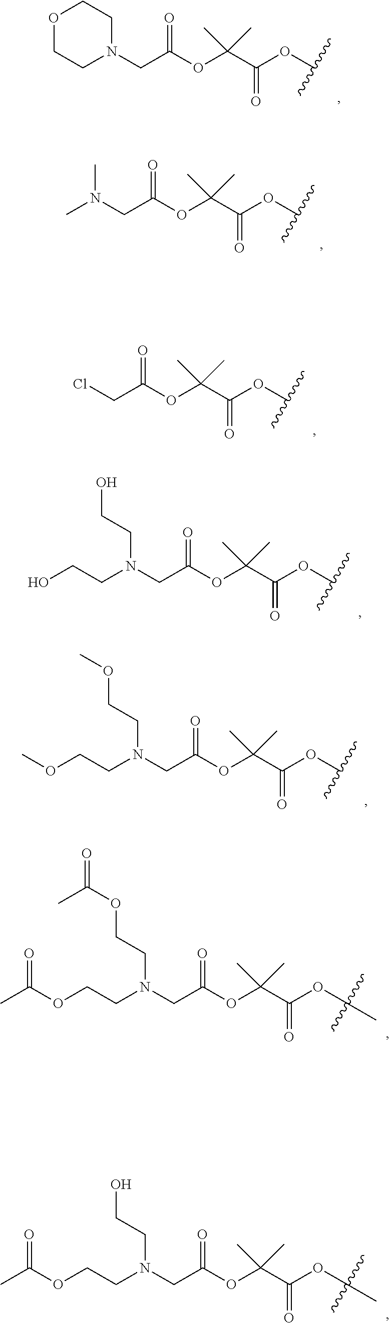

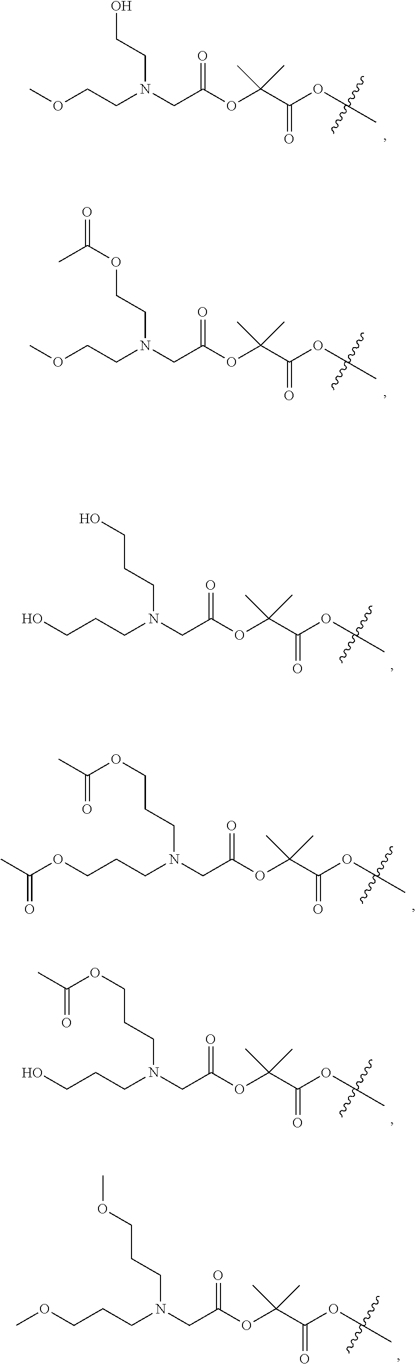

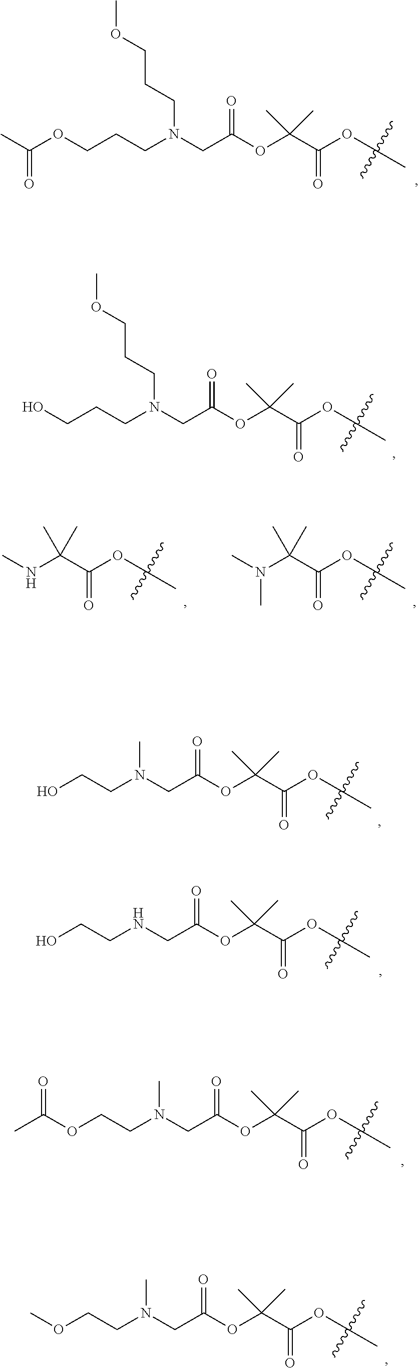

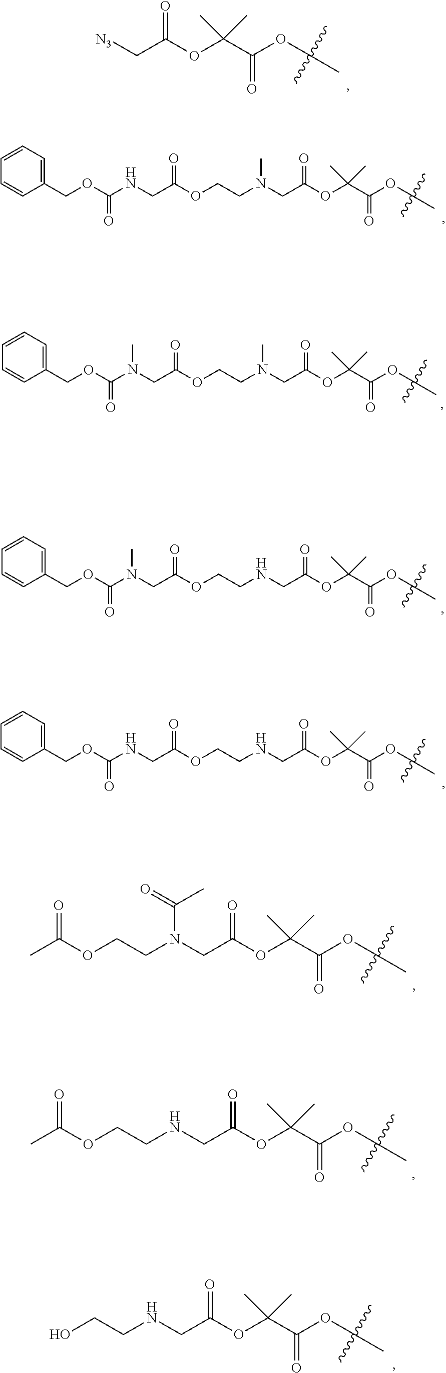

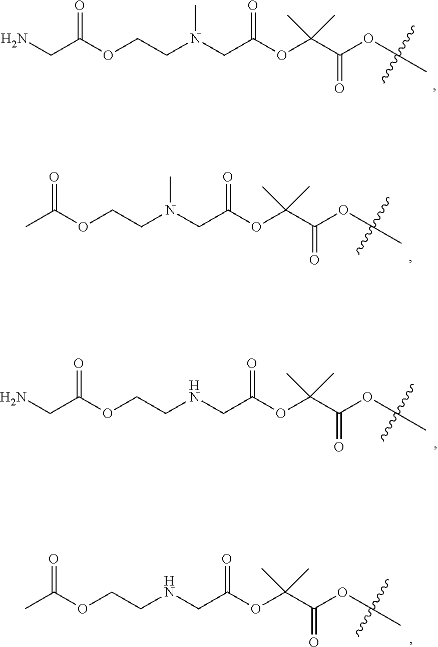

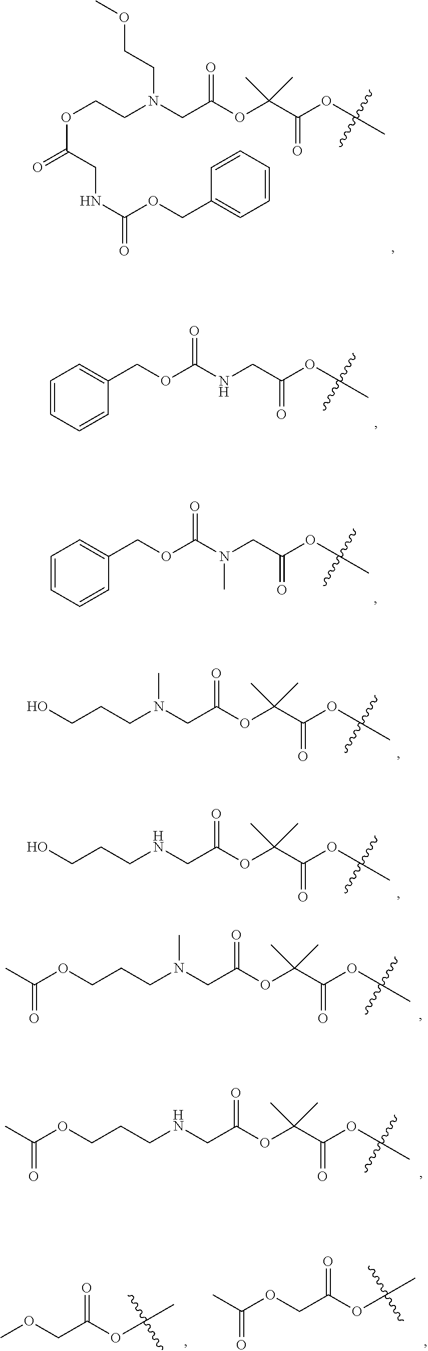

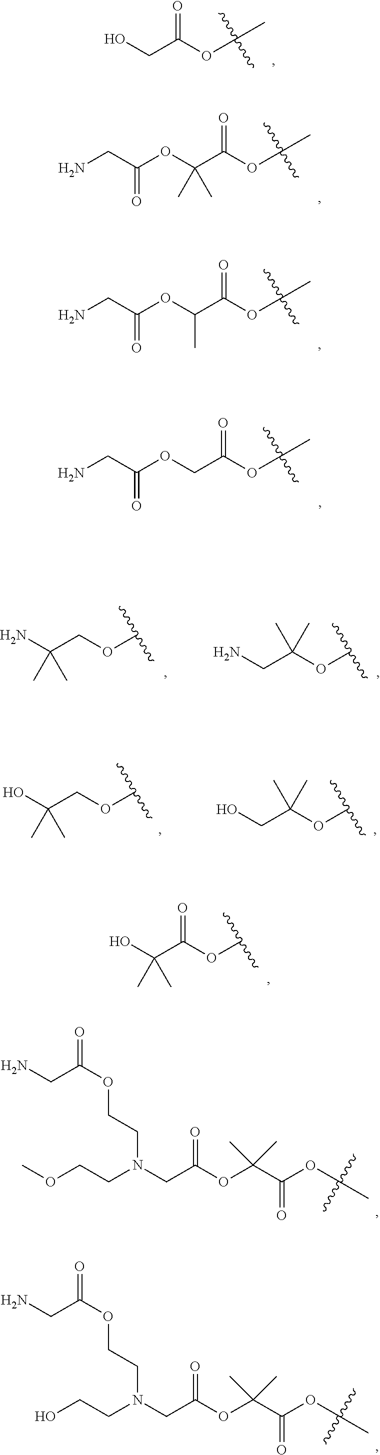

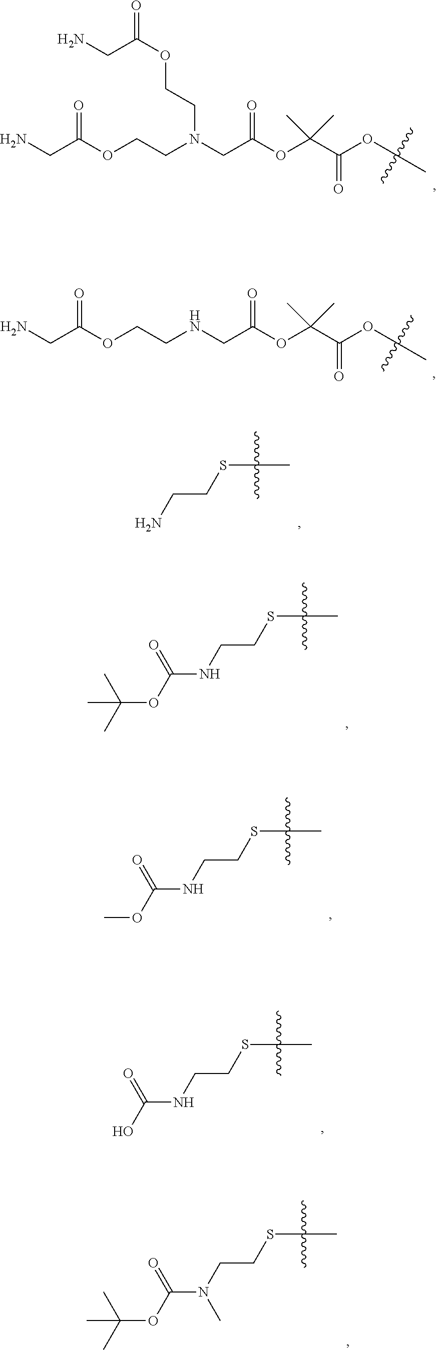

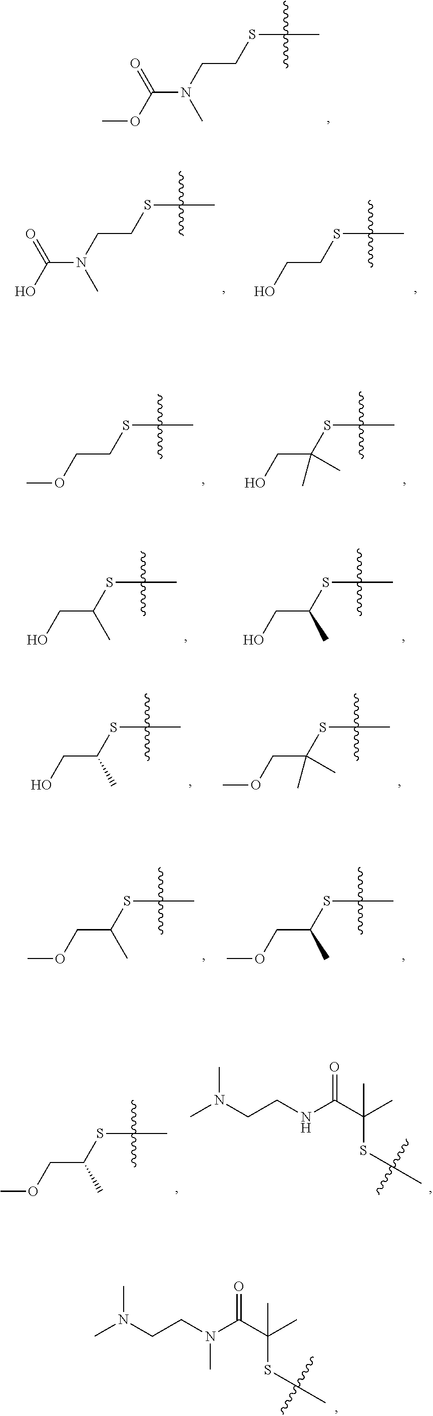

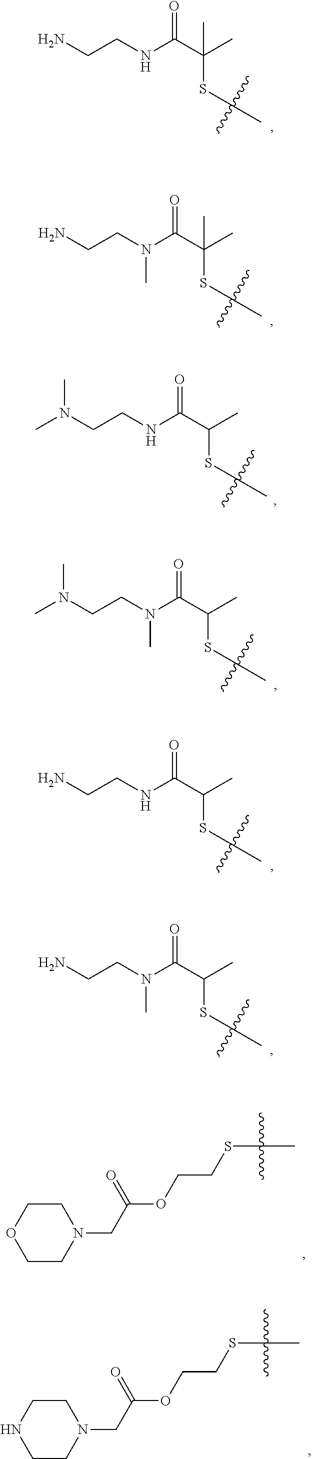

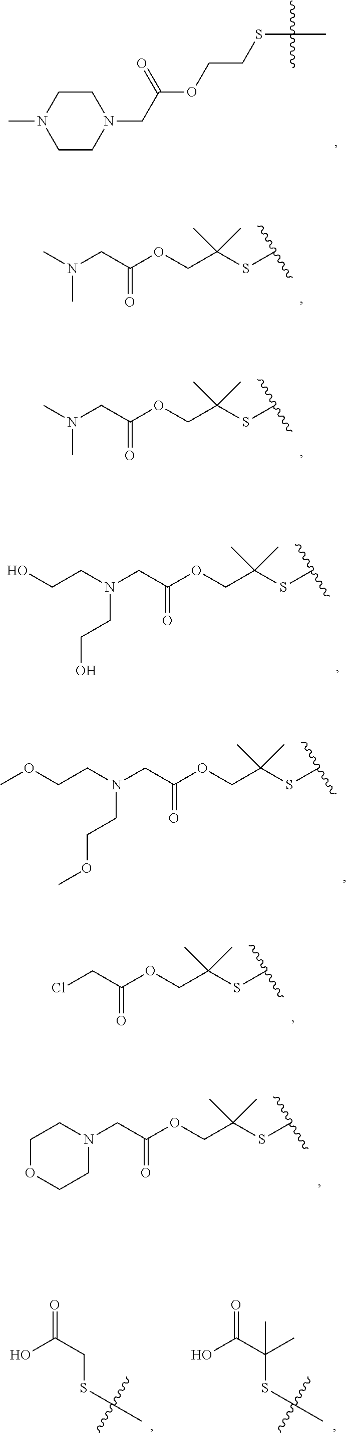

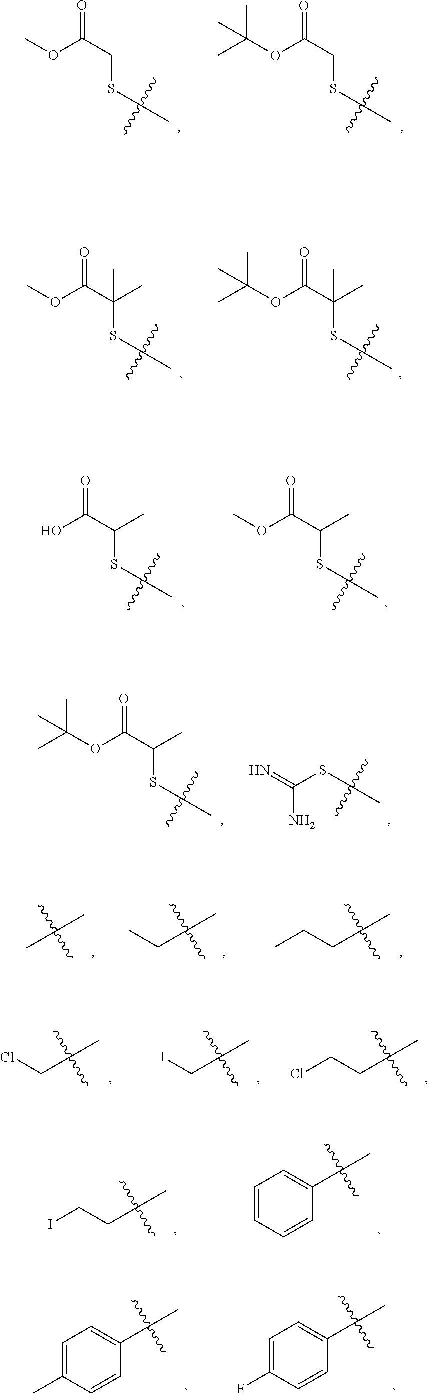

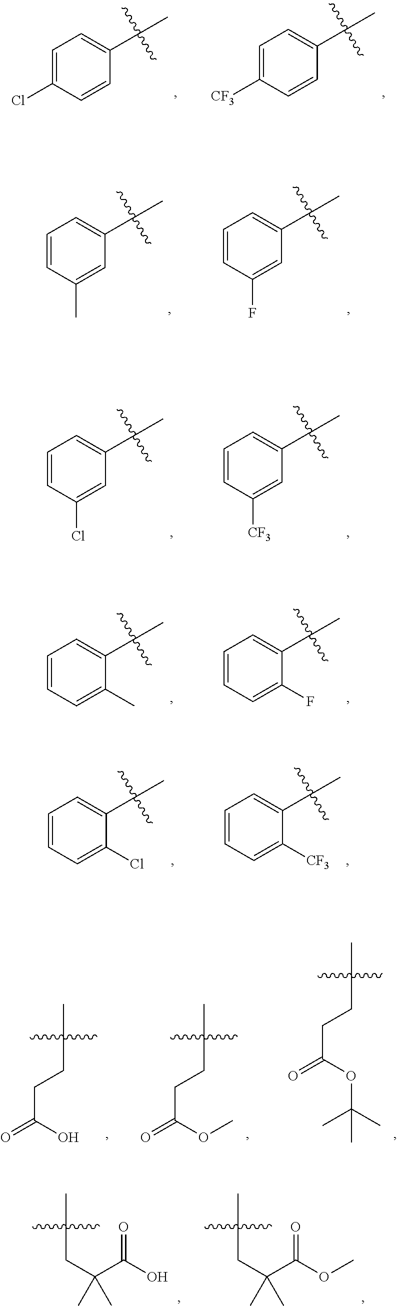

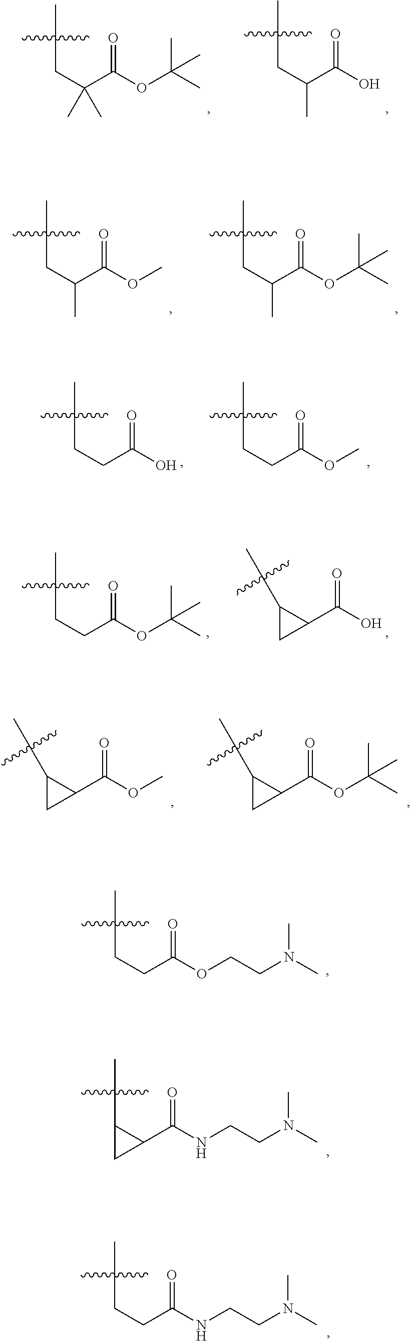

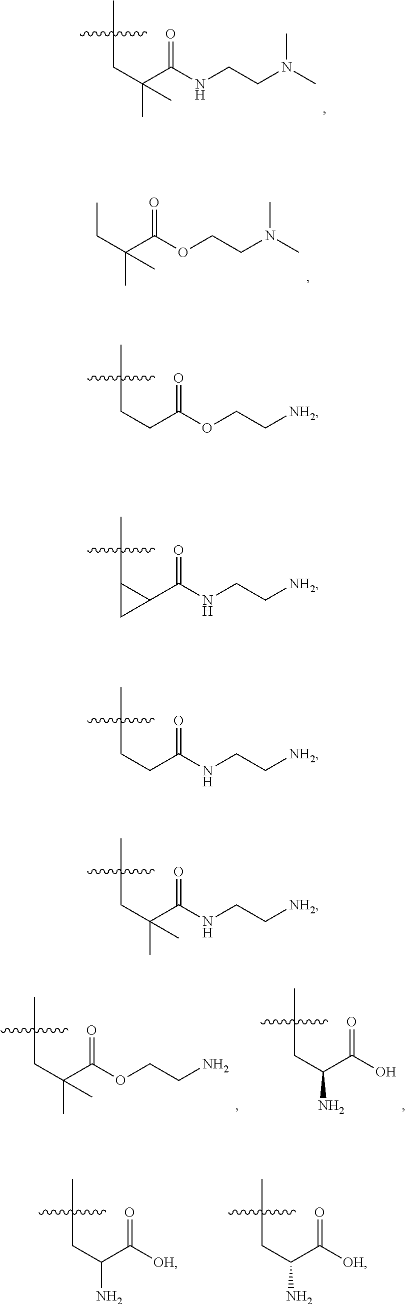

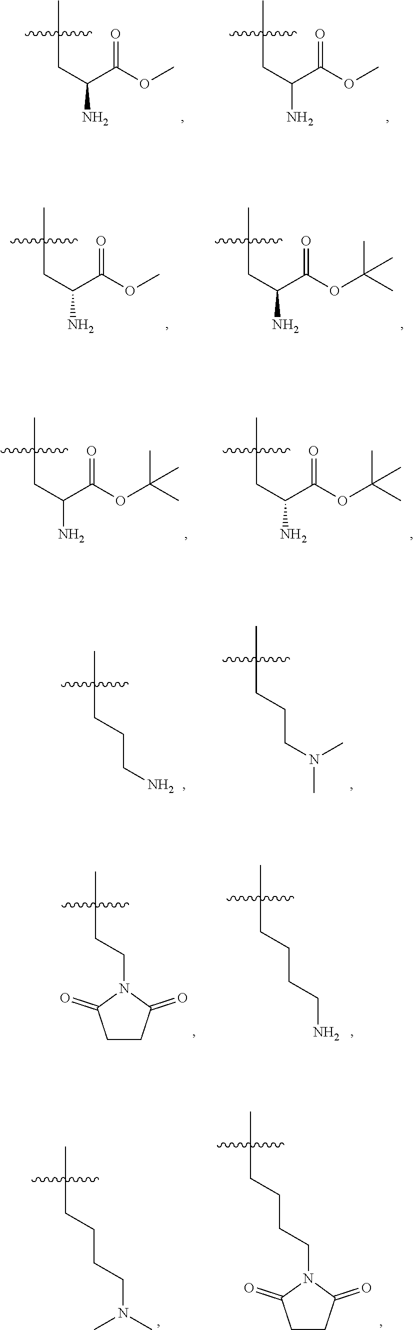

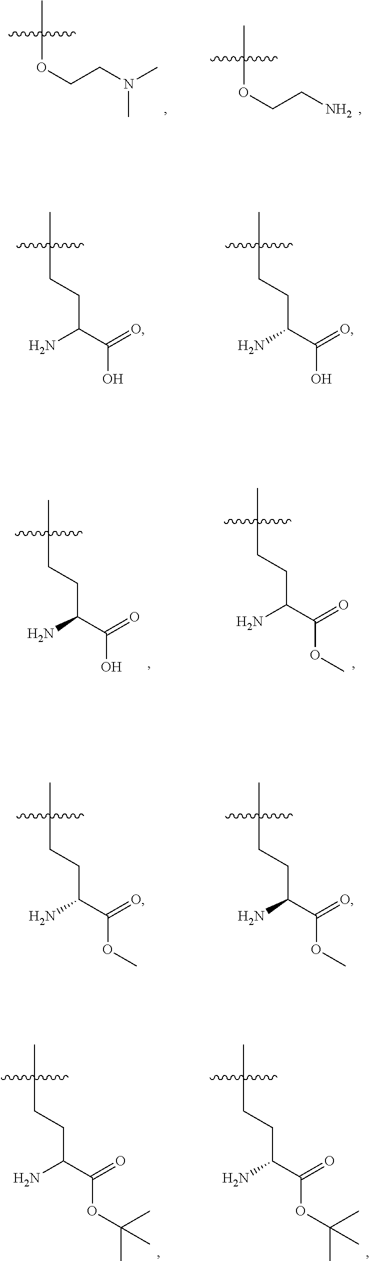

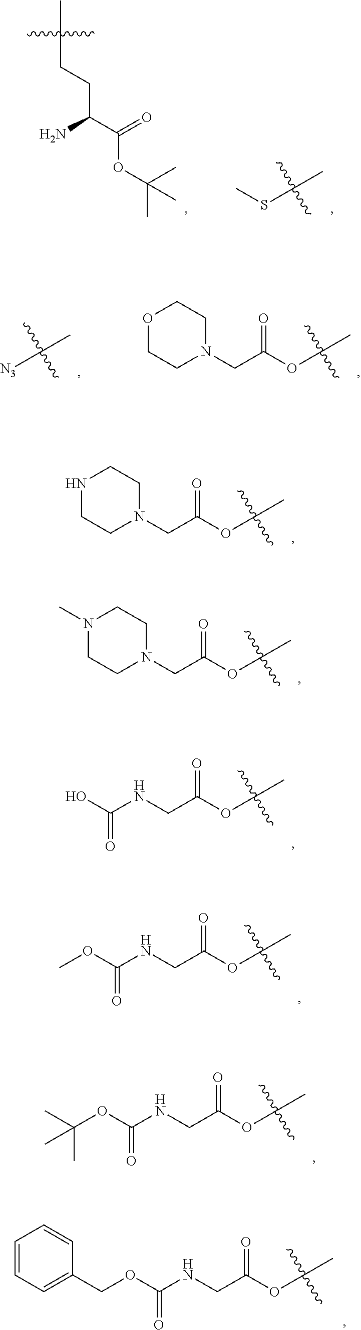

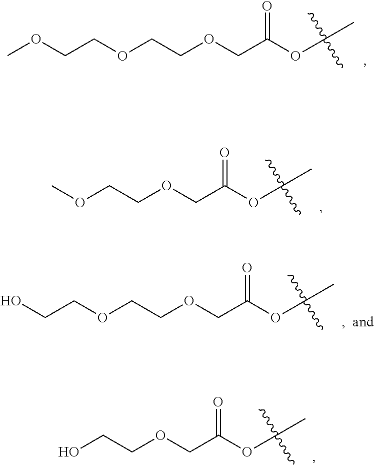

wherein [symbol] denotes the point of attachment of X to another group of the compound, e.g., an attachment of X to Z, or X to Y when Z is absent in Formula (I), or an attachment of X to the carbon atom of the —C(R^{7A})(R^{7B}) group in Formula (I-I).

The compound of Formula (I) or Formula (I-I) may be a compound of Formula (IA):

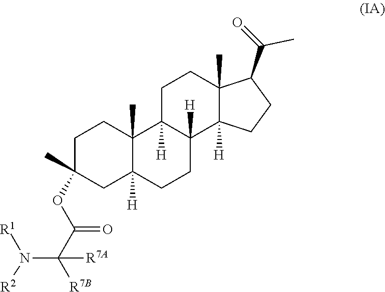

or a pharmaceutically acceptable salt, solvate, hydrate, tautomer, or stereoisomer thereof, wherein
R¹ and R² are each independently hydrogen, alkyl, alkenyl, alkynyl, heteroalkyl, cycloalkyl, heterocyclyl, aryl, heteroaryl, alkylene-cycloalkyl, alkylene-heterocyclyl, alkylene-aryl, alkylene-heteroaryl, heteroalkylene-cycloalkyl, heteroalkylene-heterocyclyl, heteroalkylene-aryl, heteroalkylene-heteroaryl, cycloalkylene-alkyl, cycloalkylene-heteroalkyl, heterocyclylene-alkyl, or heterocyclylene-heteroalkyl, wherein the alkyl, alkylene, alkenyl, alkynyl, heteroalkyl, heteroalkylene, cycloalkyl, cycloalkylene, heterocyclyl, heterocyclylene, aryl, or heteroaryl is optionally substituted;
or R¹ and R², together with the atoms to which they are attached, form a 3-7 membered cycloalkyl, heterocyclyl, aryl, or heteroaryl, wherein the cycloalkyl, heterocyclyl, aryl, or heteroaryl is optionally substituted; and
R^{7A} and R^{7B} are each independently hydrogen or optionally substituted alkyl.

In some embodiments, R¹ and R² are each independently hydrogen. In some embodiments, R¹ is hydrogen, and R² is optionally substituted alkyl (e.g., alkyl substituted with one or more alkyl, oxo, thiol, amino, hydroxyl, heterocyclyl, aryl, or heteroaryl groups). In some embodiments, R¹ is hydrogen, and R² is optionally substituted heteroalkyl (e.g., heteroalkyl substituted with one or more alkyl, oxo, thiol, amino, hydroxyl, heterocyclyl, aryl, or heteroaryl groups). In some embodiments, R^{7A} and R^{7B} are each independently hydrogen. For example, R¹, R^{7A} and R^{7B} may each independently be hydrogen, and R² may be tertiary-butyl substituted with oxo and hydroxyl groups.

The —N(R¹)(R²) group of Formula (IA) may be a moiety selected from the group consisting of

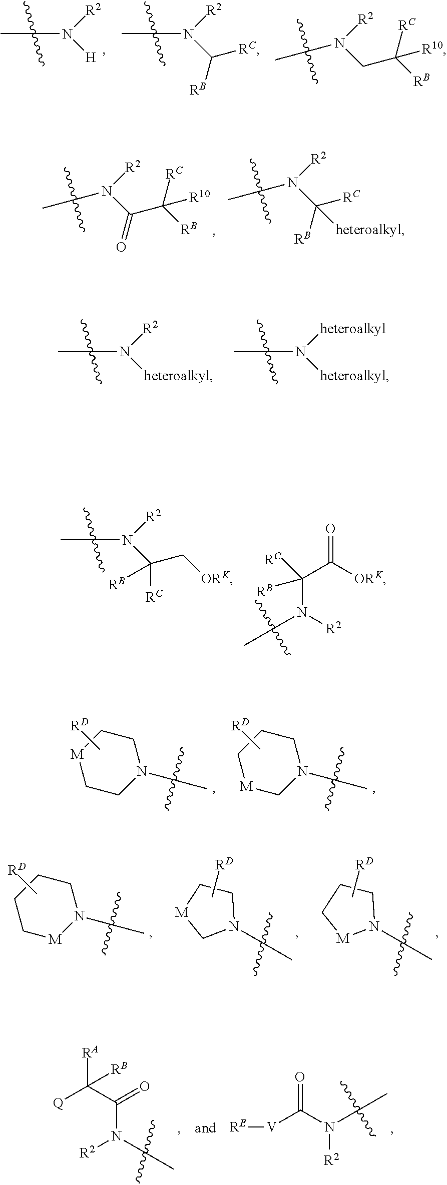

wherein
R² is hydrogen or alkyl;
M is —C(H)(R^{D})— (e.g., —CH₂—), —O—, or —N(R^{D})— (e.g., —N(Me)—);
Q is heteroalkyl or heterocyclyl, wherein the heteroalkyl or heterocyclyl is optionally substituted;
V is —O—, —N(H)—, or —N(Me)—;
R^{A}, R^{B}, R^{C}, R^{D}, R^{E} and R^{K} are each independently hydrogen, alkyl, heteroalkyl, cycloalkyl, heterocyclyl, aryl, or heteroaryl, wherein the alkyl, heteroalkyl, cycloalkyl, heterocyclyl, aryl, or heteroaryl is optionally substituted;
and [symbol] denotes the point of attachment of the nitrogen atom to the carbon atom of the —C(R^{7A})(R^{7B}) group.

Each optional substituent of a group in Formula (IA) (e.g., an optional substituent on an alkyl, alkylene, alkenyl, alkenylene, alkynyl, alkynylene, heteroalkyl, heteroalkylene, cycloalkyl, cycloalkylene, heterocyclyl, heterocyclylene, aryl, or heteroaryl group) may be any suitable substituent, e.g., a substituent described herein. For example, the optional substituent can be alkyl (e.g., C₁-C₆ alkyl), alkenyl (e.g., C₂-C₆ alkenyl), alkynyl (e.g., C₂-C₆ alkynyl), heteroalkyl (e.g., C₁-C₆ heteroalkyl), haloalkyl (e.g., C₁-C₆ haloalkyl, e.g., —CH₂Cl, —CH₂I, —(CH₂)₂Cl, —(CH₂)₂I, or —CF₃), cycloalkyl (e.g., C₃-C₈ cycloalkyl, e.g., cyclopropyl, cyclobutyl, cyclopentyl, or cyclohexyl), heterocyclyl (e.g., C₃-C₈ heterocyclyl, e.g., morpholinyl, piperazinyl, N-methylpiperazinyl, azetidinyl, or N-Boc-azetidinyl), alkylaryl (e.g., benzyl), aryl (e.g., phenyl), heteroaryl (e.g., pyrrolyl, imidazolyl, or indolyl), halogen (e.g., —F, —Cl, —Br, or —I), hydroxyl, alkylcarbonyloxy, arylcarbonyloxy, alkoxy (e.g., —OMe, —OEt, or —OBn), alkoxycarbonyloxy, aryloxycarbonyloxy, carboxylate, alkylcarbonyl, arylcarbonyl, alkoxycarbonyl, amino (e.g., —NH₂, —NHMe, or —NMe₂), aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl, and ureido), amidino, imino, sulfhydryl, alkylthio, arylthio, thiocarboxylate, sulfate, sulfinyl, sulfonyl, alkylsulfinyl, sulfonato, sulfamoyl, sulfonamido, oxo, nitro, trifluoromethyl, cyano, azido, alkyl-cycloalkyl, alkyl-heterocyclyl, alkylheteroaryl, —C(O)OH, —C(O)O-alkyl (e.g., —C(O)O-tBu or —C(O)OMe), —C(O)O— heteroalkyl, —C(O)O-alkylaryl, —OC(O)O-alkyl, —OC(O)O-heteroalkyl, —OC(O)O-alkylaryl, —C(O)NH₂, —C(O)NH-alkyl, —C(O)NH-heteroalkyl, —C(O)NH-alkylaryl, —NHC(O)O-alkyl (e.g., —NHC(O)OtBu), —NHC(O)O-heteroalkyl, —NHC(O)O-alkylaryl (e.g., —NH-Cbz), —C(O)N(alkyl)₂, —C(O)N(alkyl)(heteroalkyl), —C(O)N(heteroalkyl)₂, —N(alkyl)C(O)O-alkyl (e.g., N(Me)C(O)OtBu), —N(alkyl)C(O)O-heteroalkyl, —N(alkyl)C(O)O-alkylaryl, —N(alkyl)C(O)N(alkyl)₂, —N(alkyl)C(O)N(alkyl)(heteroalkyl), —N(alkyl)C(O)N(heteroalkyl)₂, —N(alkyl)C(O)NH-alkylaryl, ═NH, or ═N-alkyl. Cyclic groups (e.g., cycloalkyl, heterocyclyl, aryl, and heteroaryl) can be substituted at one or more ring positions with any suitable substituent, such as one of the substituents listed above.

The —N(R¹)(R²) group of Formula (IA) may be a moiety selected from the group consisting of

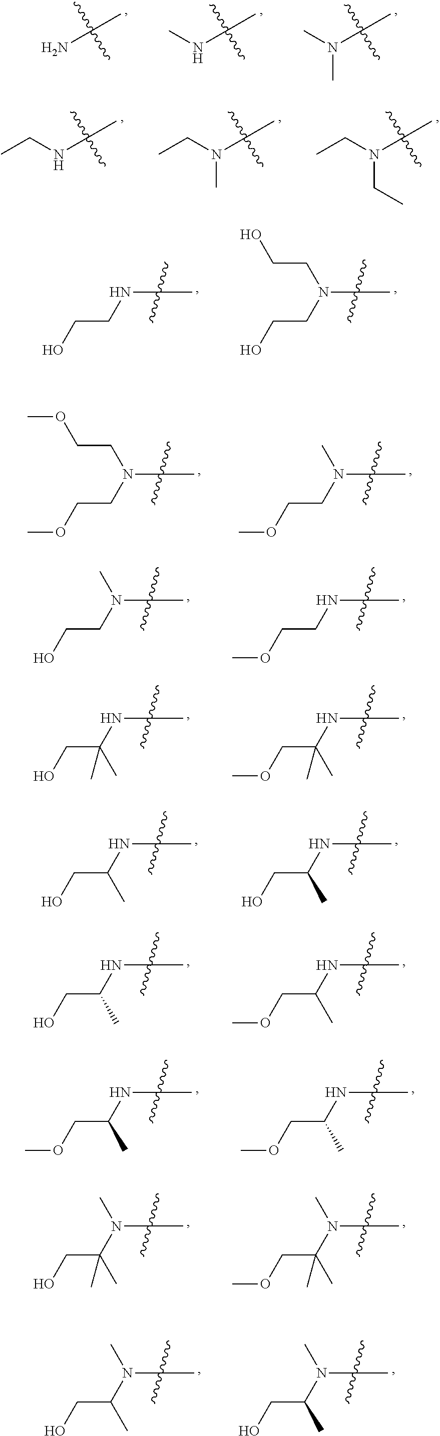

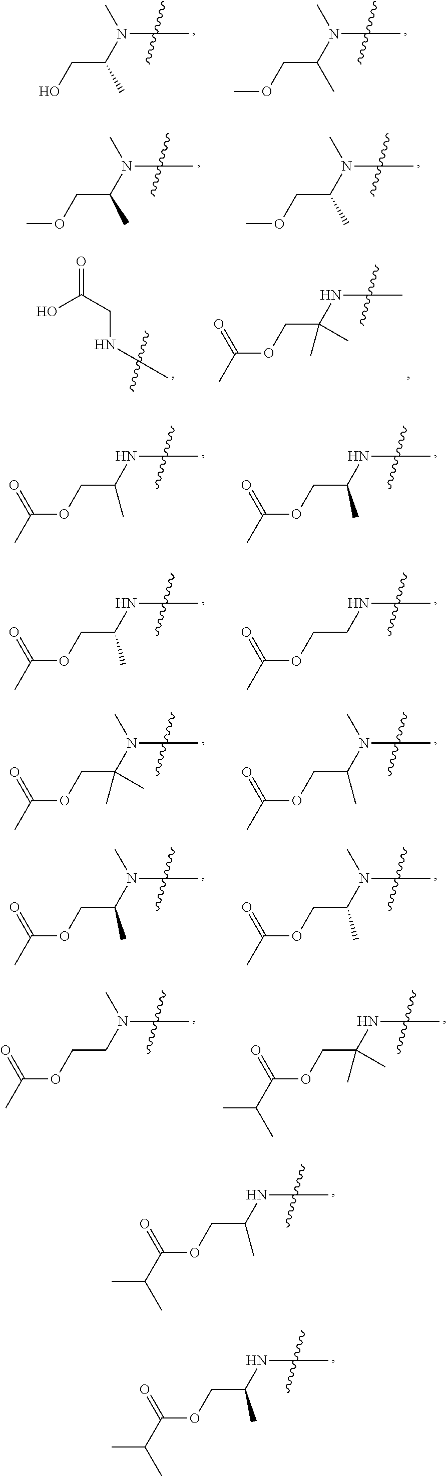

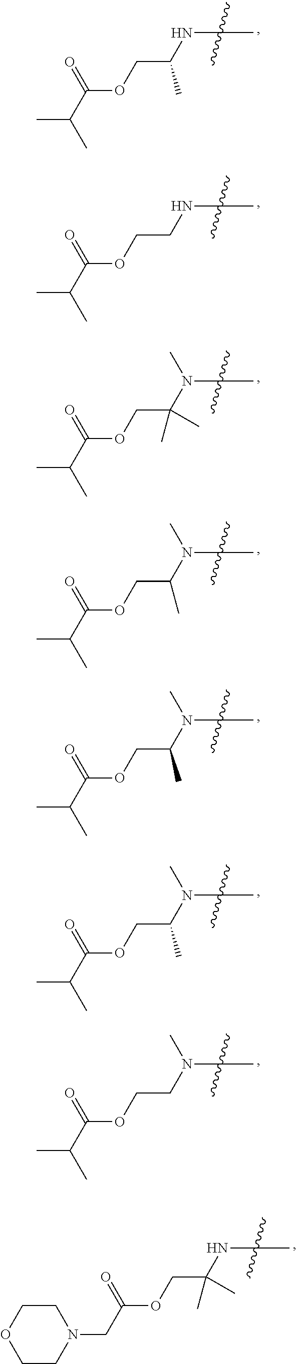

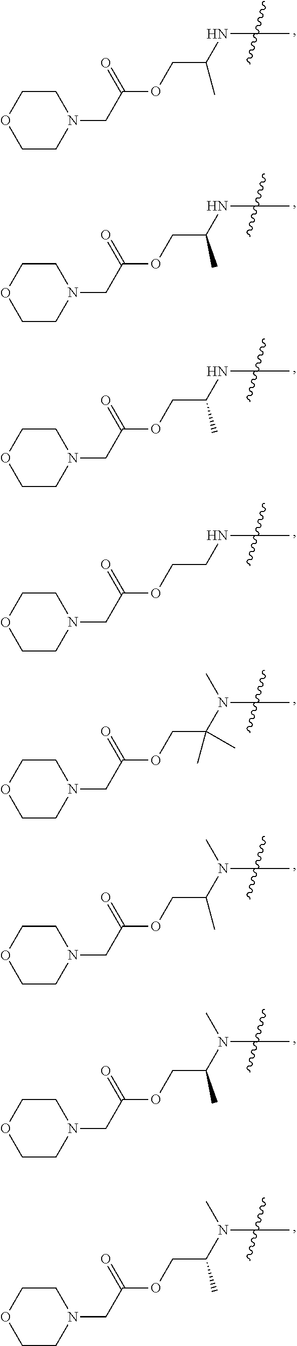

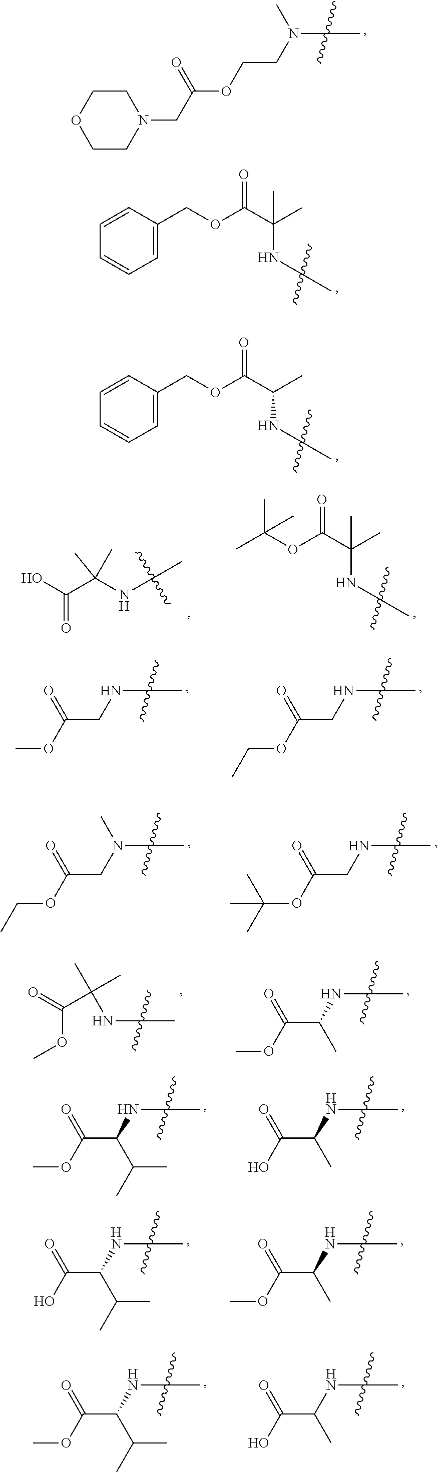

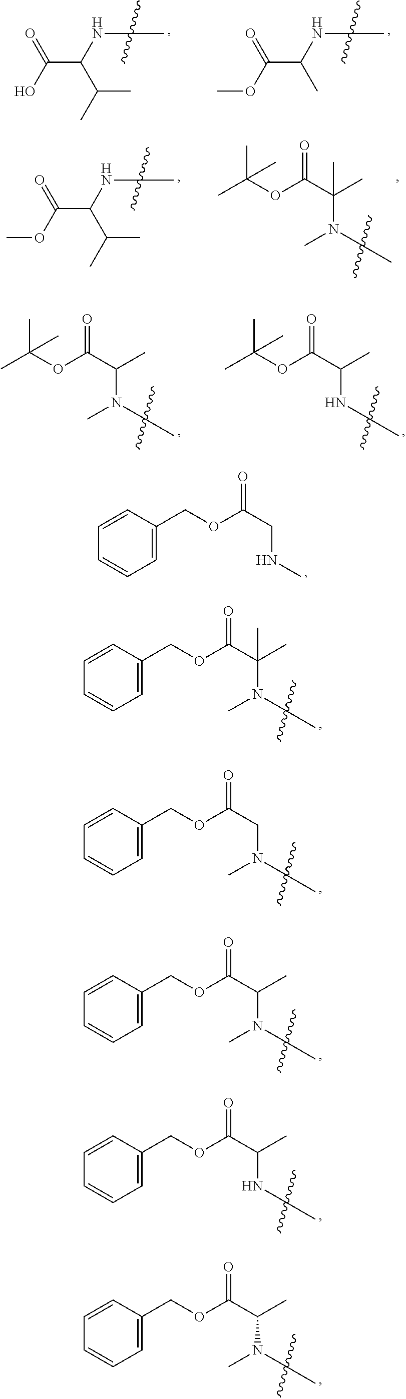

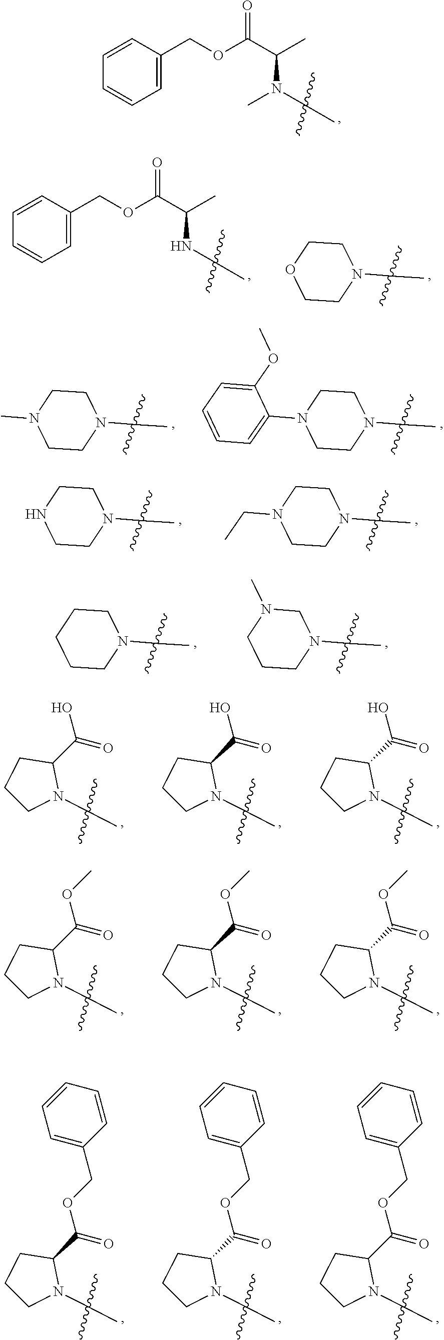

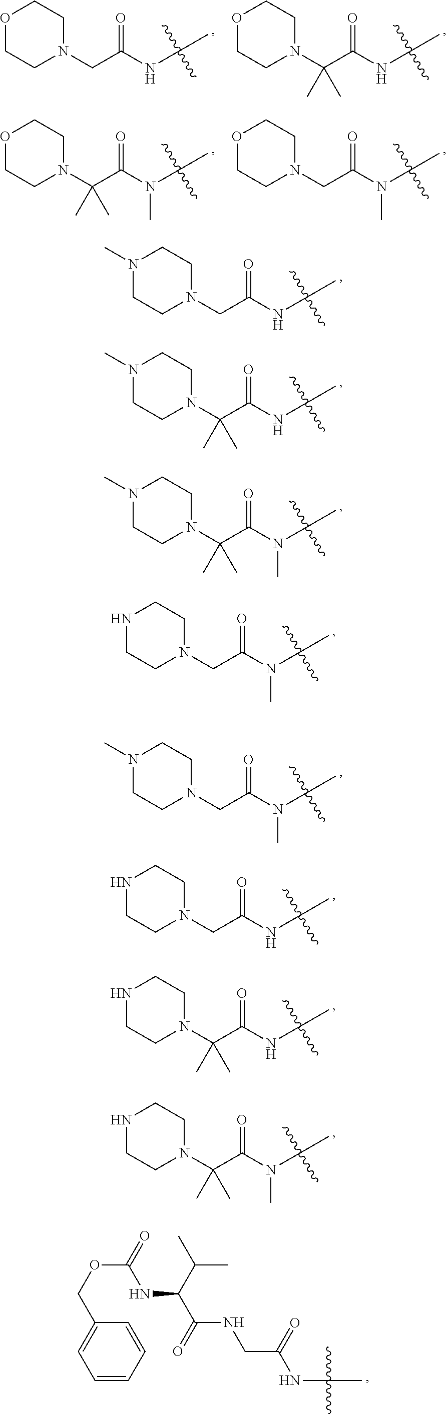

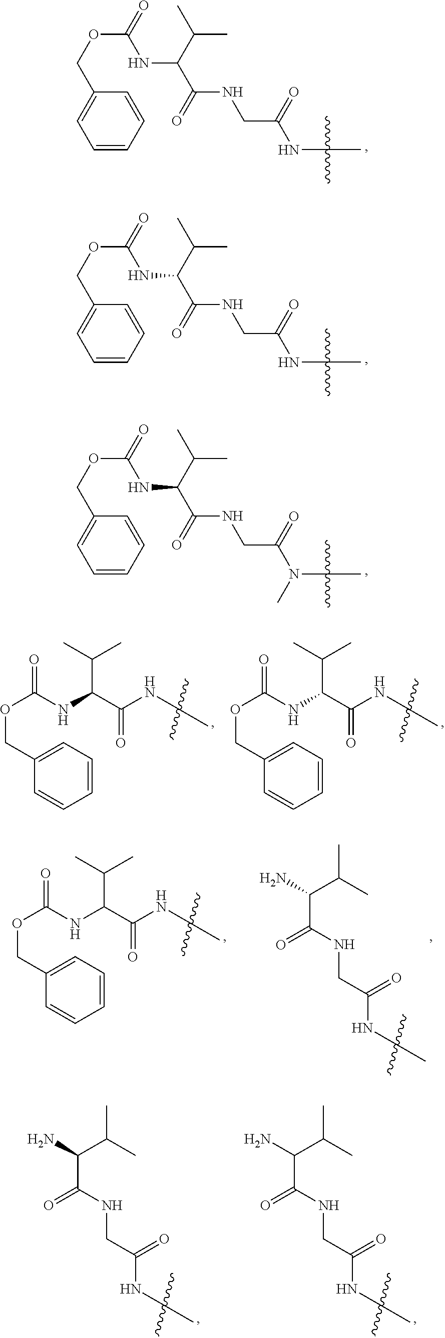

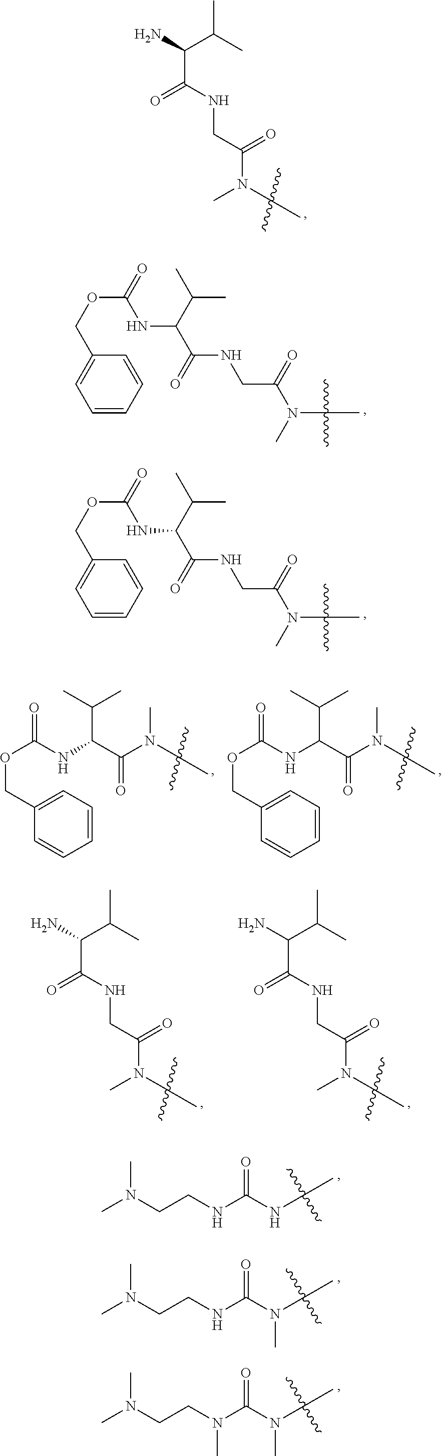

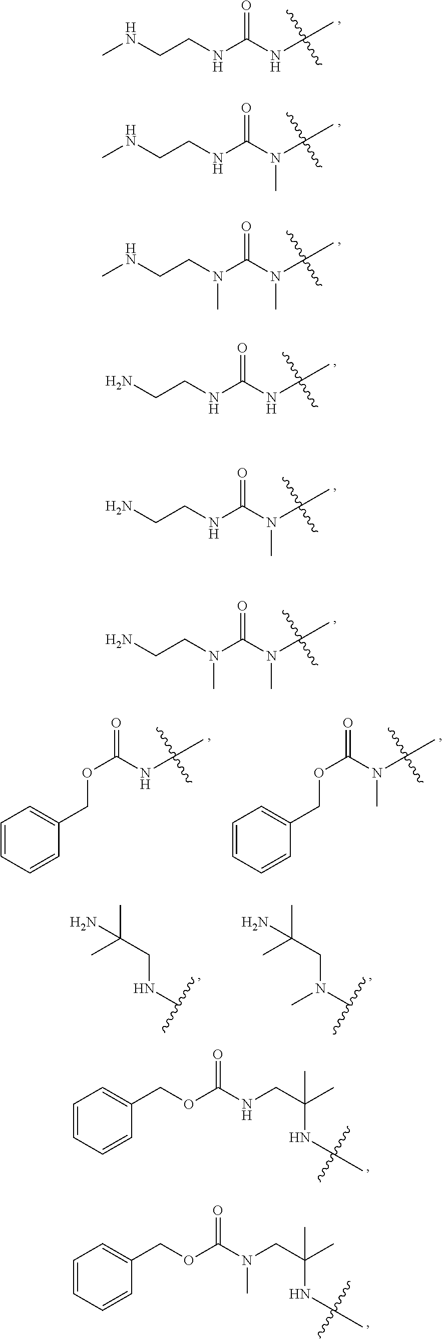

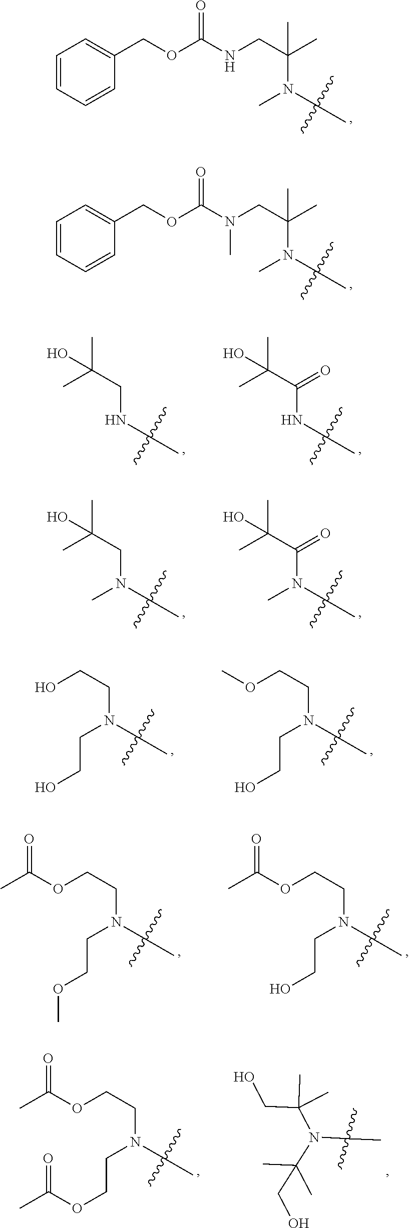

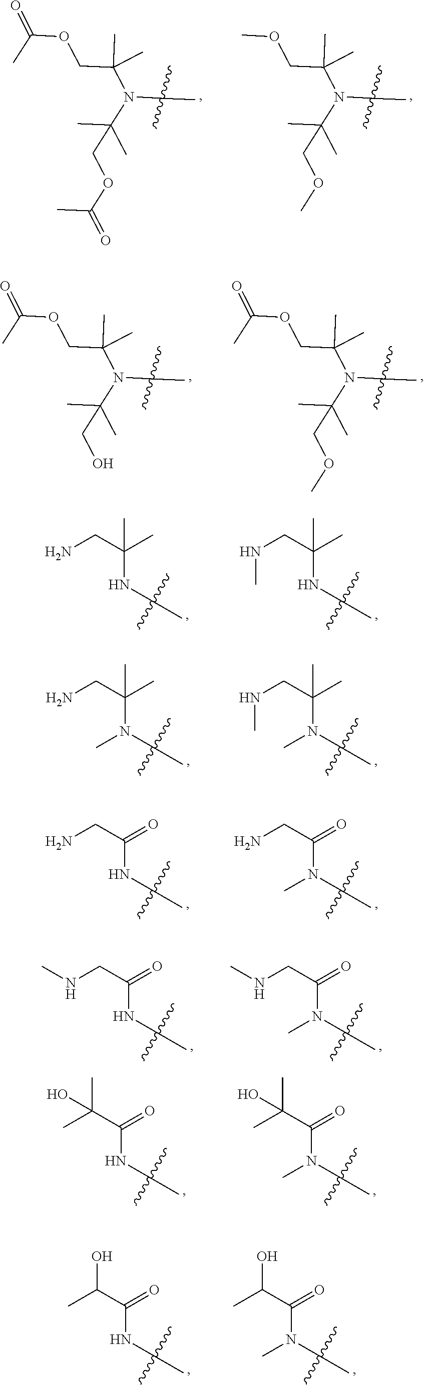

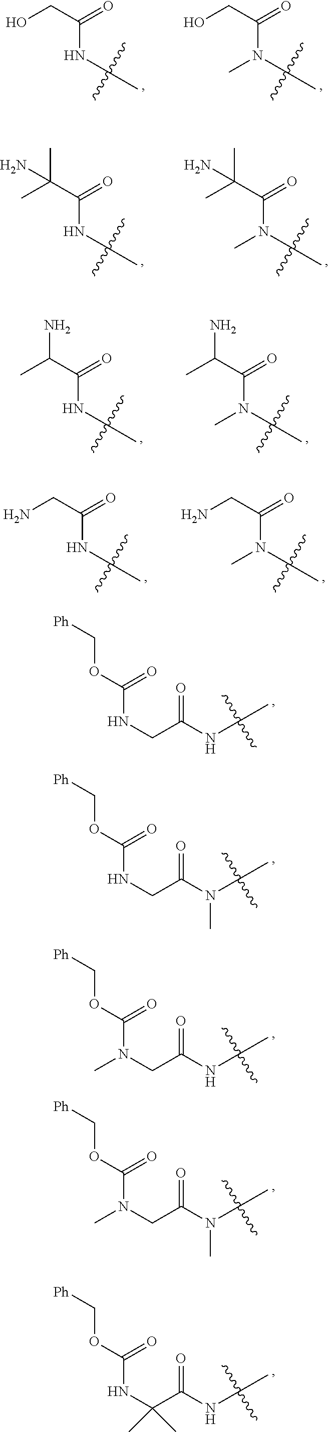

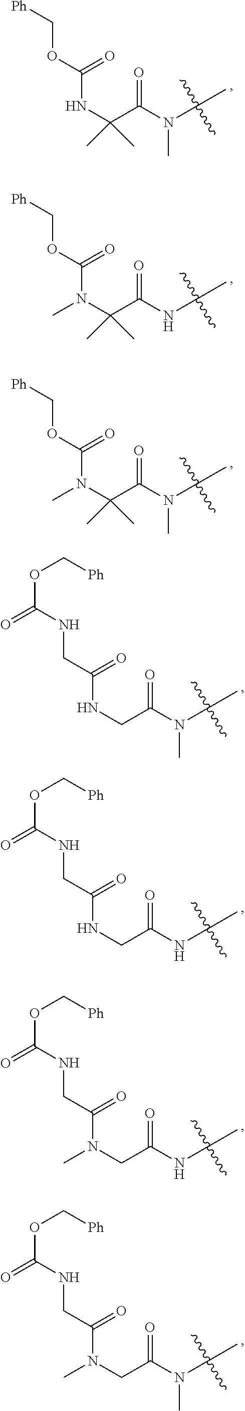

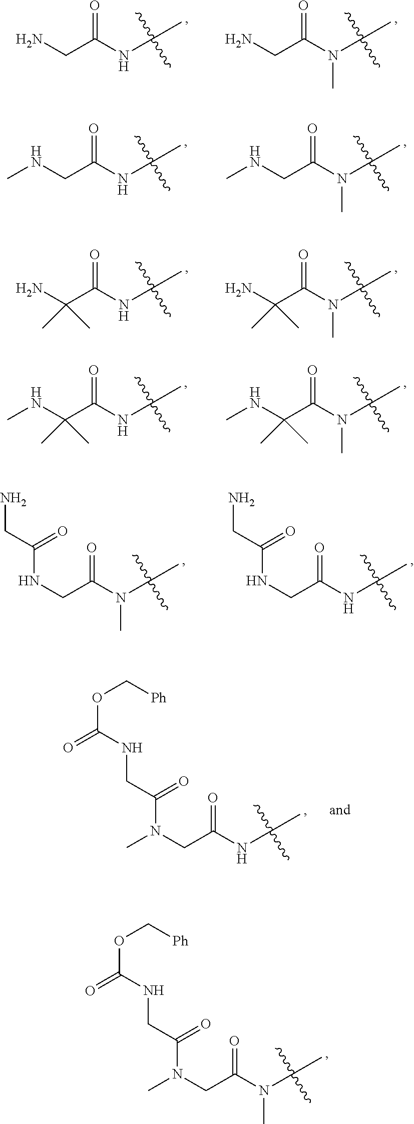

wherein [symbol] denotes the point of attachment of the nitrogen atom to the carbon atom of the —C(R^{7A})(R^{7B}) group.

The compound of Formula (I), Formula (I-I), or Formula (IA) may be a compound of Formula (IA-a):

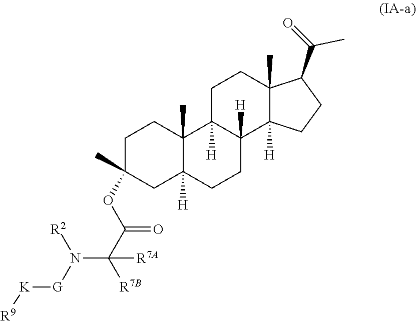

or a pharmaceutically acceptable salt, solvate, hydrate, tautomer, or stereoisomer thereof, wherein
G is absent, —C(O)—, or —C(R^{8A})(R^{8B})— (e.g., —CH₂—),
K is alkylene, heteroalkylene, —C(O)—, —OC(O)—, —C(O)O—, —O—, —O—(CH₂)—, or —(CH₂)—O—, wherein the alkylene or heteroalkylene is optionally substituted;
R², R^{7A}, R^{7B}, R^{8A}, and R^{8B} are each independently hydrogen or optionally substituted alkyl; and
R⁹ is hydrogen, alkyl, alkenyl, alkynyl, heteroalkyl, cycloalkyl, heterocyclyl, aryl, or heteroaryl, wherein the alkyl, alkenyl, alkynyl, heteroalkyl, cycloalkyl, heterocyclyl, aryl, or heteroaryl is optionally substituted.

It will be understood that when K is —OC(O)— or —(CH₂)—O—, the —OC(O)— or —(CH₂)—O— group may be connected to G and R⁹ groups in either orientation (in other words, these groups may be thought of as being equivalent to —C(O)O— and —O—(CH₂)—, respectively). For example, when G is —C(R^{8A})(R^{8B})— and K is —OC(O)—, it will be understood that both

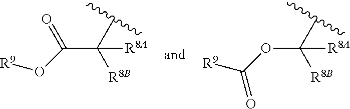

are described. In some embodiments, G is —C(R^{8A})(R^{8B})— and K is —OC(O)—, and the carbonyl group in —OC(O)— is bonded to G, i.e.,

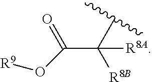

Each optional substituent of a group in Formula (IA-a) (e.g., an optional substituent on an alkyl, alkylene, alkenyl, alkenylene, alkynyl, alkynylene, heteroalkyl, heteroalkylene, cycloalkyl, cycloalkylene, heterocyclyl, heterocyclylene, aryl, or heteroaryl group) may be any suitable substituent, e.g., a substituent described herein. For example, the optional substituent can be alkyl (e.g., C₁-C₆ alkyl), alkenyl (e.g., C₂-C₆ alkenyl), alkynyl (e.g., C₂-C₆ alkynyl), heteroalkyl (e.g., C₁-C₆ heteroalkyl), haloalkyl (e.g., C₁-C₆ haloalkyl, e.g., —CH₂Cl, —CH₂I, —(CH₂)₂Cl, —(CH₂)₂I, or —CF₃), cycloalkyl (e.g., C₃-C₈ cycloalkyl, e.g., cyclopropyl, cyclobutyl, cyclopentyl, or cyclohexyl), heterocyclyl (e.g., C₃-C₈ heterocyclyl, e.g., morpholinyl, piperazinyl, N-methylpiperazinyl, azetidinyl, or N-Boc-azetidinyl), alkylaryl (e.g., benzyl), aryl (e.g., phenyl), heteroaryl (e.g., pyrrolyl, imidazolyl, or indolyl), halogen (e.g., —F, —Cl, —Br, or —I), hydroxyl, alkylcarbonyloxy, arylcarbonyloxy, alkoxy (e.g., —OMe, —OEt, or —OBn), alkoxycarbonyloxy, aryloxycarbonyloxy, carboxylate, alkylcarbonyl, arylcarbonyl, alkoxycarbonyl, amino (e.g., —NH₂, —NHMe, or —NMe₂), aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl, and ureido), amidino, imino, sulfhydryl, alkylthio, arylthio, thiocarboxylate, sulfate, sulfinyl, sulfonyl, alkylsulfinyl, sulfonato, sulfamoyl, sulfonamido, oxo, nitro, trifluoromethyl, cyano, azido, alkyl-cycloalkyl, alkyl-heterocyclyl, alkylheteroaryl, —C(O)OH, —C(O)O-alkyl (e.g., —C(O)O-tBu or —C(O)OMe), —C(O)O— heteroalkyl, —C(O)O-alkylaryl, —OC(O)O-alkyl, —OC(O)O-heteroalkyl, —OC(O)O-alkylaryl, —C(O)NH₂, —C(O)NH-alkyl, —C(O)NH-heteroalkyl, —C(O)NH-alkylaryl, —NHC(O)O-alkyl (e.g., —NHC(O)OtBu), —NHC(O)O-heteroalkyl, —NHC(O)O-alkylaryl (e.g., —NH-Cbz), —C(O)N(alkyl)₂, —C(O)N(alkyl)(heteroalkyl), —C(O)N(heteroalkyl)₂, —N(alkyl)C(O)O-alkyl (e.g., N(Me)C(O)OtBu), —N(alkyl)C(O)O-heteroalkyl, —N(alkyl)C(O)O-alkylaryl, —N(alkyl)C(O)N(alkyl)₂, —N(alkyl)C(O)N(alkyl)(heteroalkyl), —N(alkyl)C(O)N(heteroalkyl)₂, —N(alkyl)C(O)NH-alkylaryl, ═NH, or ═N-alkyl. Cyclic groups (e.g., cycloalkyl, heterocyclyl, aryl, and heteroaryl) can be substituted at one or more ring positions with any suitable substituent, such as one of the substituents listed above.

In some embodiments G is —C(R^{8A})(R^{8B})—, e.g., —C(CH₃)₂—. In some embodiments, K is —OC(O)—. In some embodiments, R² is hydrogen. In some embodiments, R^{7A} and R^{7B} are each independently hydrogen. In some embodiments, R⁹ is hydrogen. In some embodiments, R^{8A} and R^{8B} are each independently alkyl (e.g., methyl). For example, G may be —C(CH₃)₂—. In some embodiments, G is-C(CH₃)₂—, K is —OC(O)—, and R², R^{7A} and R^{7B} are each independently hydrogen.

The compound of Formula (I), Formula (I-I), Formula (IA), or Formula (IA-a) may be a compound of Formula (IA-b):

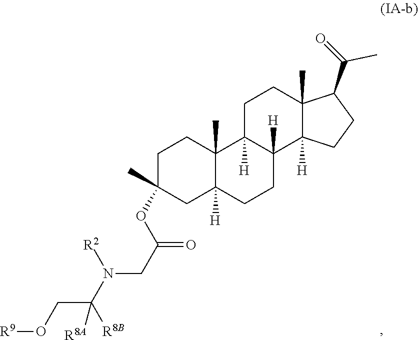

or a pharmaceutically acceptable salt, solvate, hydrate, tautomer, or stereoisomer thereof, wherein
R², R^{8A}, and R^{8B} are each independently hydrogen or optionally substituted alkyl; and
R⁹ is hydrogen, alkyl, alkenyl, alkynyl, heteroalkyl, cycloalkyl, heterocyclyl, aryl, or heteroaryl, wherein the alkyl, alkenyl, alkynyl, heteroalkyl, cycloalkyl, heterocyclyl, aryl, or heteroaryl is optionally substituted.

In some embodiments, R² is hydrogen. In some embodiments, R^{8A} and R^{8B} are each independently alkyl (e.g., methyl). In some embodiments, R^{8A} and R^{8B} are each independently methyl. For example, R² may be hydrogen and R^{8A} and R^{8B} may each be independently methyl. In some embodiments, R⁹ is hydrogen.

In some embodiments, R² is hydrogen, R^{8A} and R^{8B} are each independently methyl, and R⁹ is hydrogen.

The compound of Formula (I) or Formula (I-I) may be a compound of Formula (IB):

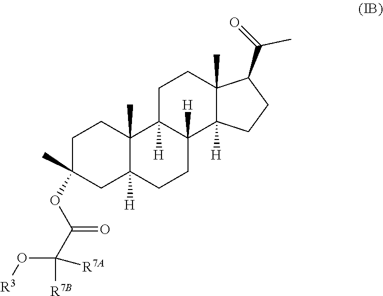

or a pharmaceutically acceptable salt, solvate, hydrate, tautomer, or stereoisomer thereof, wherein
R³ is hydrogen, alkyl, alkenyl, alkynyl, heteroalkyl, cycloalkyl, heterocyclyl, aryl, heteroaryl, alkylene-cycloalkyl, alkylene-heterocyclyl, alkylene-aryl, alkylene-heteroaryl, heteroalkylene-cycloalkyl, heteroalkylene-heterocyclyl, heteroalkylene-aryl, heteroalkylene-heteroaryl, cycloalkylene-alkyl, cycloalkylene-heteroalkyl, heterocyclylene-alkyl, or heterocyclylene-heteroalkyl, wherein the alkyl, alkylene, alkenyl, alkynyl, heteroalkyl, heteroalkylene, cycloalkyl, cycloalkylene, heterocyclyl, heterocyclylene, aryl, or heteroaryl is optionally substituted; and
R^{7A} and R^{7B} are each independently hydrogen or optionally substituted alkyl.

The —O—(R³) group of Formula (IB) may be a moiety selected from the group consisting of

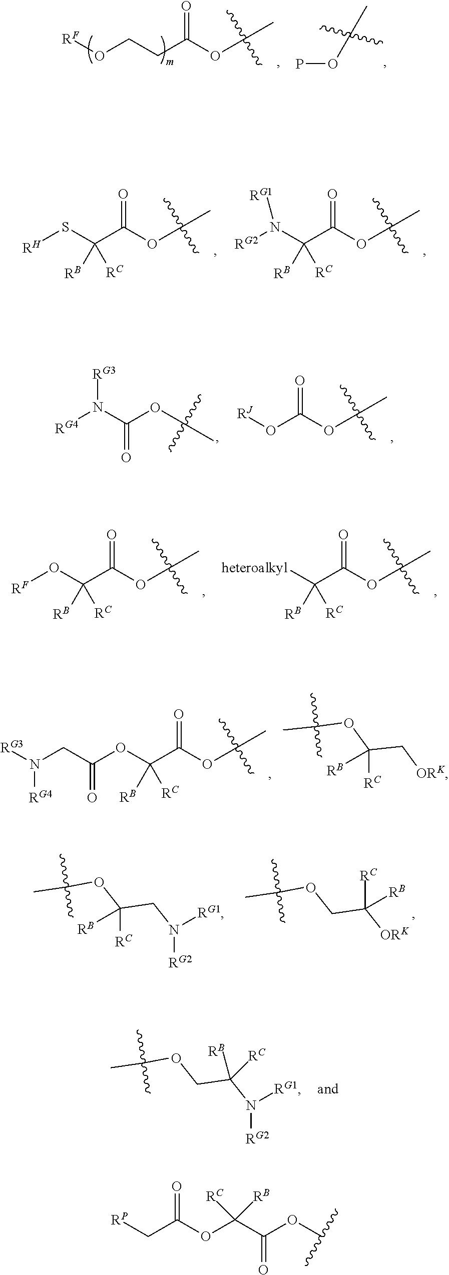

wherein
P is an amino acid residue (e.g., glycine, alanine, valine, leucine, or proline), that may be a D-amino acid residue or L-amino acid residue, and is optionally substituted (e.g., at a terminal nitrogen atom, e.g., by a carbonate group, e.g., a carboxybenzyl (Cbz) group),
or P is a dipeptide moiety (e.g., a leucine-leucine moiety) that may comprise D-amino acids, L-amino acids, or a combination thereof, and is optionally substituted (e.g., at one or more nitrogen atoms, e.g., at a terminal nitrogen atom, e.g., by a carbonate group, e.g., a carboxybenzyl (Cbz) group);
R^{B} and R^{C} are each independently hydrogen, alkyl, heteroalkyl, cycloalkyl, heterocyclyl, aryl, or heteroaryl, wherein the alkyl, heteroalkyl, cycloalkyl, heterocyclyl, aryl, or heteroaryl is optionally substituted;
R^{F} and R^{H} are each independently hydrogen or alkyl;
R^{G1} and R^{G2} are each independently hydrogen, alkyl, or an amino acid residue (e.g., glycine, alanine, valine, leucine or proline), that may be a D-amino acid residue or L-amino acid residue, and is optionally substituted (e.g., at a terminal nitrogen atom, e.g., by a carbonate group, e.g., a carboxybenzyl (Cbz) group),
or R^{G1} and R^{G2}, together with the atoms to which they are attached, form a 3-7 membered cycloalkyl, heterocyclyl, aryl, or heteroaryl, wherein the cycloalkyl, heterocyclyl, aryl, or heteroaryl is optionally substituted;
R^{G3} and R^{G4} are each independently hydrogen, alkyl, or heteroalkyl, wherein the alkyl or heteroalkyl is optionally substituted;
or R^{G3} and R^{G4}, together with the atoms to which they are attached, form a 3-7 membered cycloalkyl, heterocyclyl, aryl, or heteroaryl, wherein the cycloalkyl, heterocyclyl, aryl, or heteroaryl is optionally substituted;
R^{J} is hydrogen, alkyl, alkylene-heterocyclyl, heteroalkyl, or heterocyclyl, wherein the alkyl, alkylene, heteroalkyl, or heterocyclyl is optionally substituted;
R^{K} is hydrogen, alkyl, heteroalkyl, cycloalkyl, heterocyclyl, aryl, or heteroaryl, wherein the alkyl, heteroalkyl, cycloalkyl, heterocyclyl, aryl, or heteroaryl is optionally substituted;
R^{P} is halo, —NH-alkyl, —NH-heteroalkyl, —N-(alkyl)₂ (e.g., —N(Me)₂), —N-(heteroalkyl)₂ (e.g., —N(CH₂CH₂OH)₂), or heterocyclyl (e.g., morpholinyl);
m is an integer of 1-5; and
[symbol] denotes the point of attachment of the oxygen atom to the carbon atom of the —C(R^{7A})(R^{7B}) group.

Each optional substituent of a group in Formula (IB) (e.g., an optional substituent on an alkyl, alkylene, alkenyl, alkenylene, alkynyl, alkynylene, heteroalkyl, heteroalkylene, cycloalkyl, cycloalkylene, heterocyclyl, heterocyclylene, aryl, or heteroaryl group) may be any suitable substituent, e.g., a substituent described herein. For example, the optional substituent can be alkyl (e.g., C₁-C₆ alkyl), alkenyl (e.g., C₂-C₆ alkenyl), alkynyl (e.g., C₂-C₆ alkynyl), heteroalkyl (e.g., C₁-C₆ heteroalkyl), haloalkyl (e.g., C₁-C₆ haloalkyl, e.g., —CH₂Cl, —CH₂I, —(CH₂)₂Cl, —(CH₂)₂I, or —CF₃), cycloalkyl (e.g., C₃-C₈ cycloalkyl, e.g., cyclopropyl, cyclobutyl, cyclopentyl, or cyclohexyl), heterocyclyl (e.g., C₃-C₈ heterocyclyl, e.g., morpholinyl, piperazinyl, N-methylpiperazinyl, azetidinyl, or N-Boc-azetidinyl), alkylaryl (e.g., benzyl), aryl (e.g., phenyl), heteroaryl (e.g., pyrrolyl, imidazolyl, or indolyl), halogen (e.g., —F, —Cl, —Br, or —I), hydroxyl, alkylcarbonyloxy, arylcarbonyloxy, alkoxy (e.g., —OMe, —OEt, or —OBn), alkoxycarbonyloxy, aryloxycarbonyloxy, carboxylate, alkylcarbonyl, arylcarbonyl, alkoxycarbonyl, amino (e.g., —NH₂, —NHMe, or —NMe₂), aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl, and ureido), amidino, imino, sulfhydryl, alkylthio, arylthio, thiocarboxylate, sulfate, sulfinyl, sulfonyl, alkylsulfinyl, sulfonato, sulfamoyl, sulfonamido, oxo, nitro, trifluoromethyl, cyano, azido, alkyl-cycloalkyl, alkyl-heterocyclyl, alkylheteroaryl, —C(O)OH, —C(O)O-alkyl (e.g., —C(O)O-tBu or —C(O)OMe), —C(O)O— heteroalkyl, —C(O)O-alkylaryl, —OC(O)O-alkyl, —OC(O)O-heteroalkyl, —OC(O)O-alkylaryl, —C(O)NH₂, —C(O)NH-alkyl, —C(O)NH-heteroalkyl, —C(O)NH-alkylaryl, —NHC(O)O-alkyl (e.g., —NHC(O)OtBu), —NHC(O)O-heteroalkyl, —NHC(O)O-alkylaryl (e.g., —NH-Cbz), —C(O)N(alkyl)₂, —C(O)N(alkyl)(heteroalkyl), —C(O)N(heteroalkyl)₂, —N(alkyl)C(O)O-alkyl (e.g., N(Me)C(O)OtBu), —N(alkyl)C(O)O-heteroalkyl, —N(alkyl)C(O)O-alkylaryl, —N(alkyl)C(O)N(alkyl)₂, —N(alkyl)C(O)N(alkyl)(heteroalkyl), —N(alkyl)C(O)N(heteroalkyl)₂, —N(alkyl)C(O)NH-alkylaryl, ═NH, or ═N-alkyl. Cyclic groups (e.g., cycloalkyl, heterocyclyl, aryl, and heteroaryl) can be substituted at one or more ring positions with any suitable substituent, such as one of the substituents listed above.

The —O—(R³) group of Formula (IB) may be a moiety selected from the group consisting of

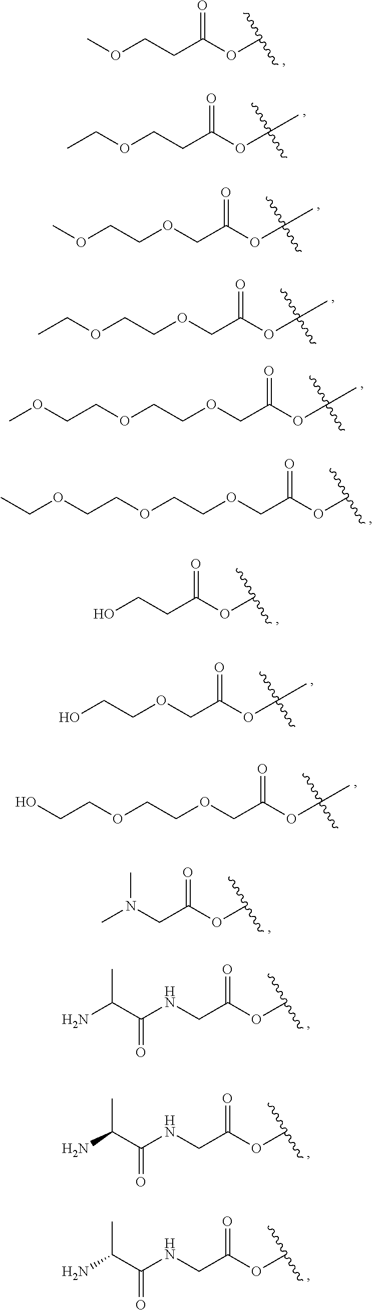

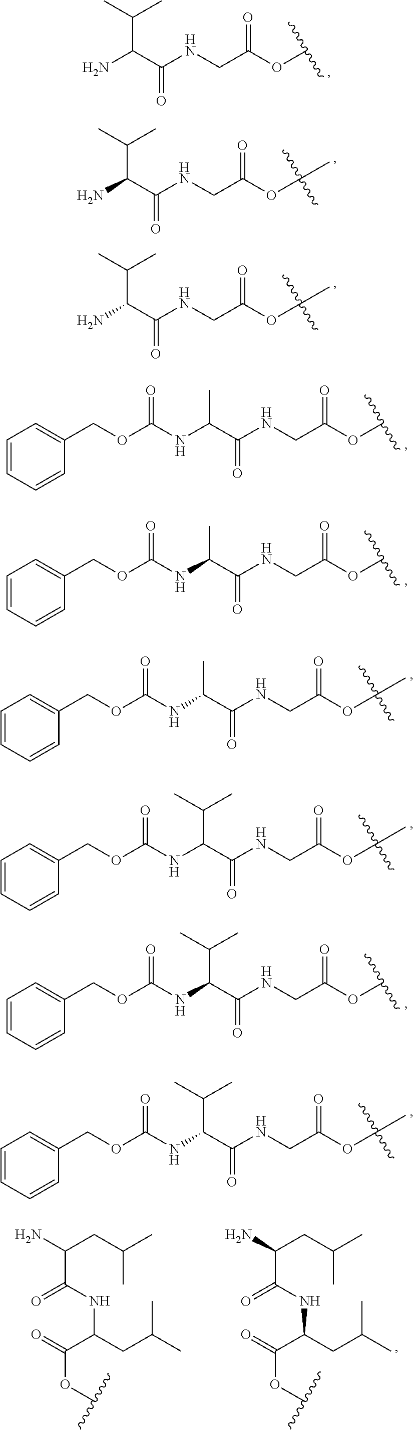

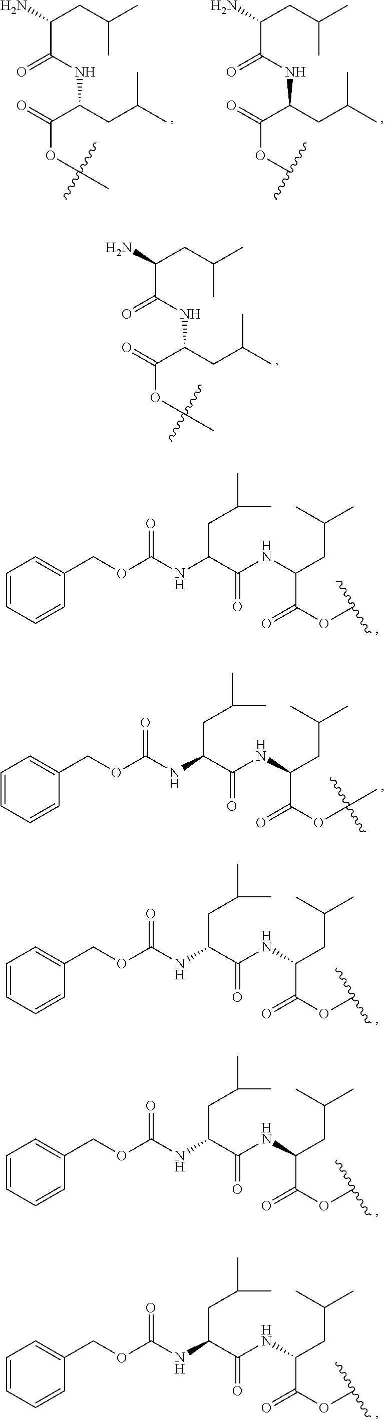

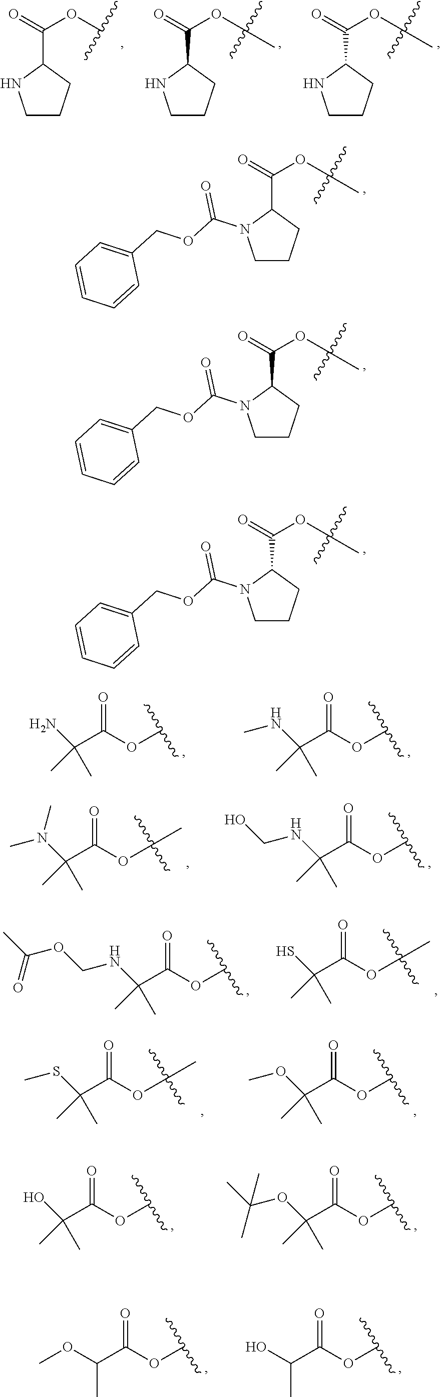

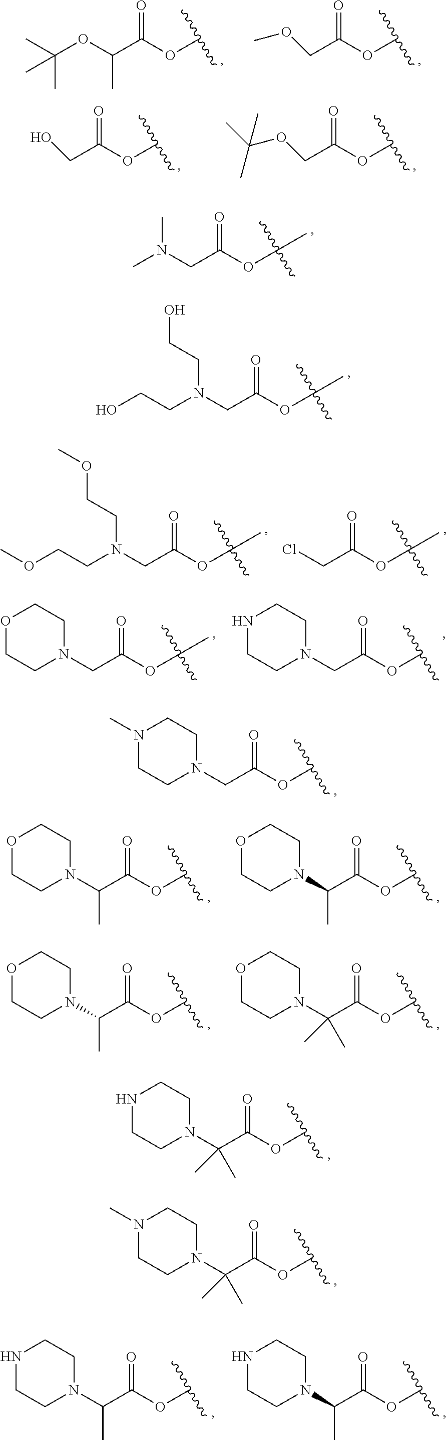

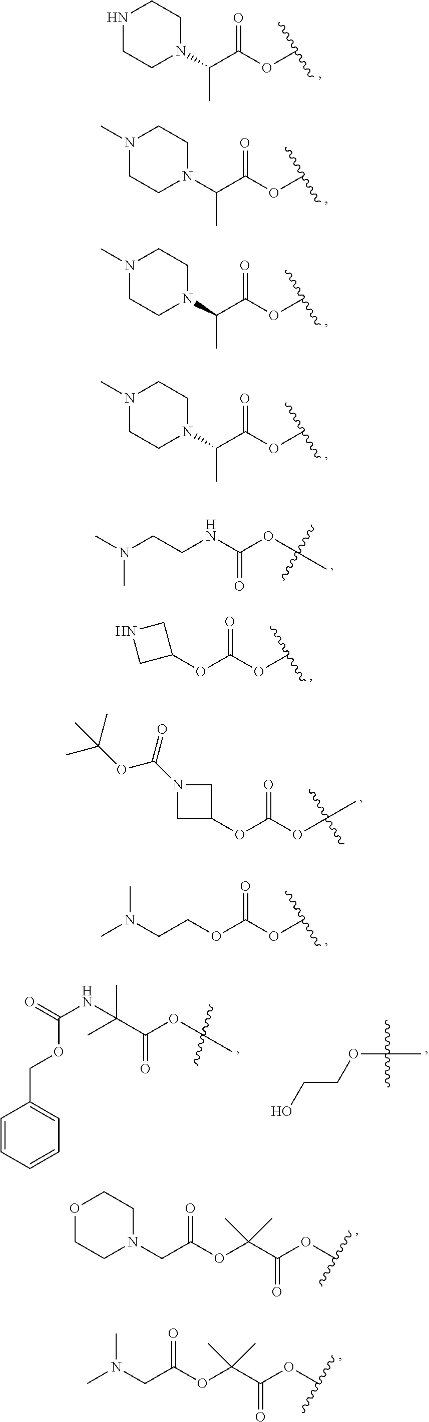

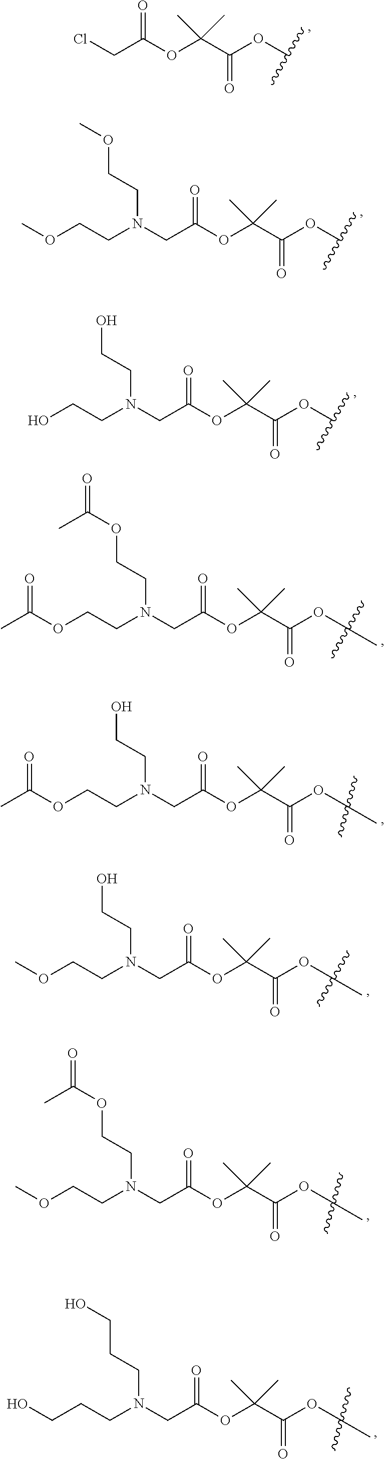

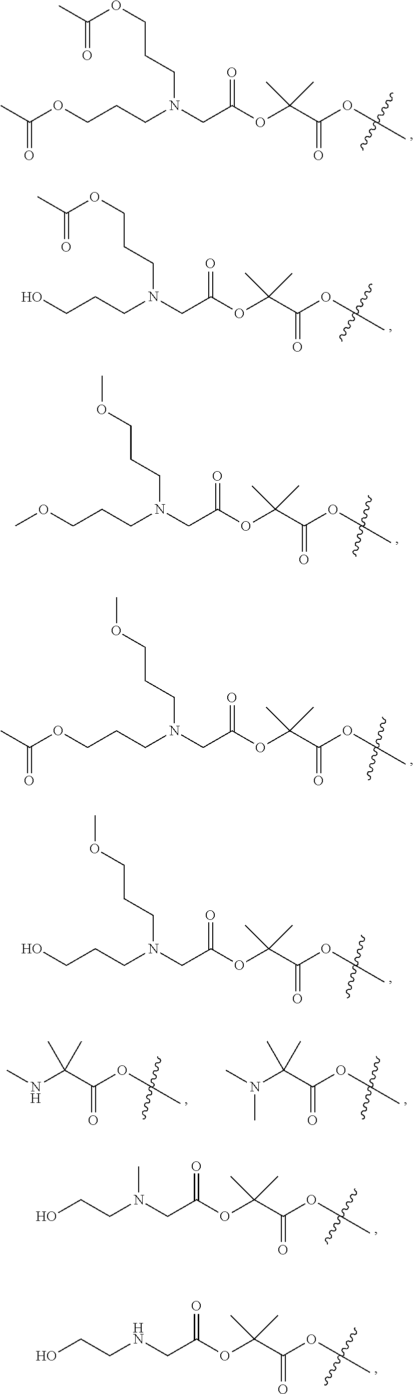

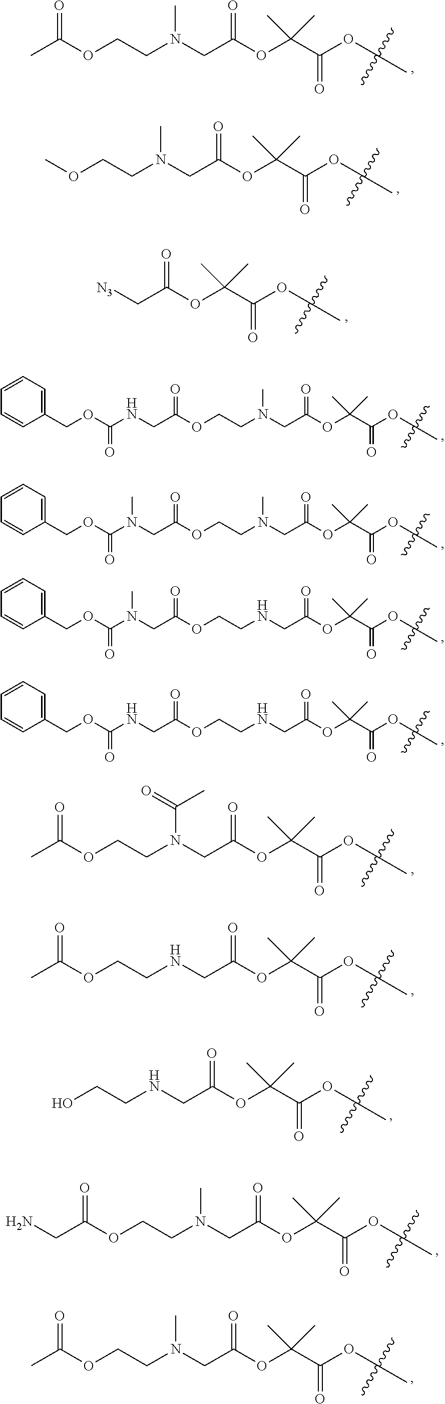

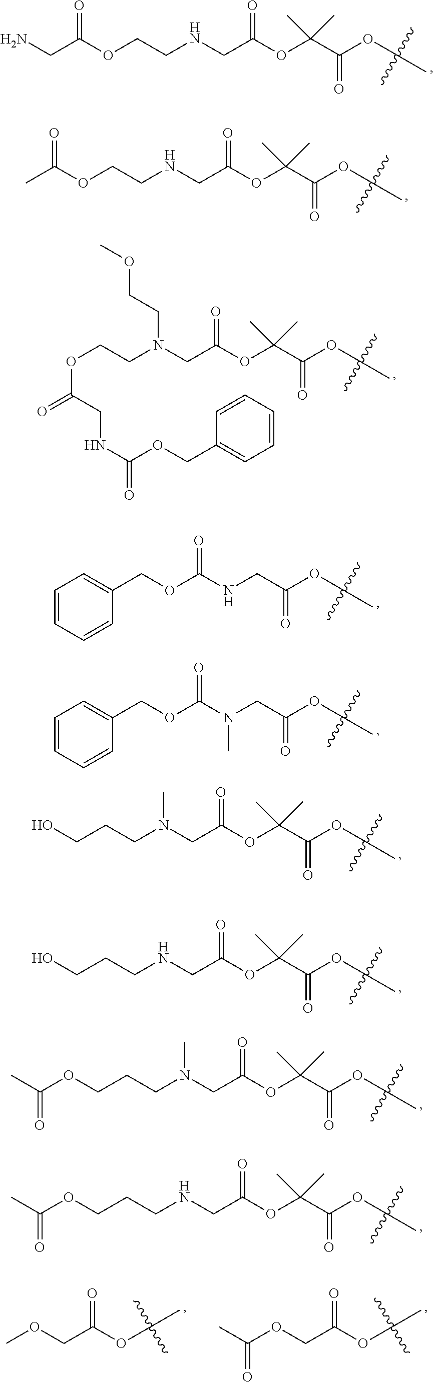

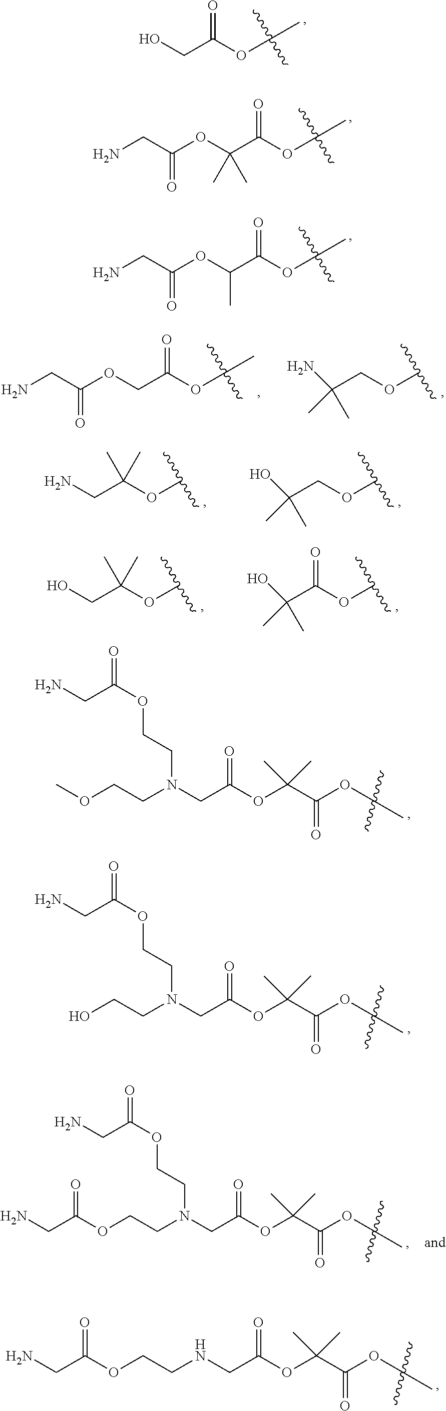

wherein [symbol] denotes the point of attachment of the oxygen atom to the carbon atom of the —C(R^{7A})(R^{7B}) group.

The compound of Formula (I) or Formula (I-I) may be a compound of Formula (IC):

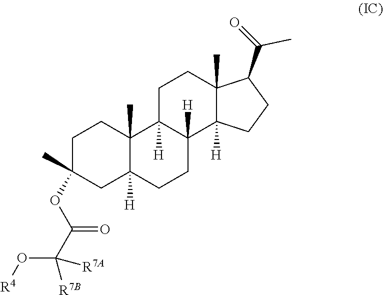

or a pharmaceutically acceptable salt, solvate, hydrate, tautomer, or stereoisomer thereof, wherein
R⁴ is hydrogen, alkyl, alkenyl, alkynyl, heteroalkyl, cycloalkyl, heterocyclyl, aryl, heteroaryl, alkylene-cycloalkyl, alkylene-heterocyclyl, alkylene-aryl, alkylene-heteroaryl, heteroalkylene-cycloalkyl, heteroalkylene-heterocyclyl, heteroalkylene-aryl, or heteroalkylene-heteroaryl, wherein the alkyl, alkylene, alkenyl, alkynyl, heteroalkyl, heteroalkylene, cycloalkyl, cycloalkylene, heterocyclyl, heterocyclylene, aryl, or heteroaryl is optionally substituted; and
R^{7A} and R^{7B} are each independently hydrogen or optionally substituted alkyl.

The —S—(R⁴) group in Formula (IC) may be a moiety selected from the group consisting of

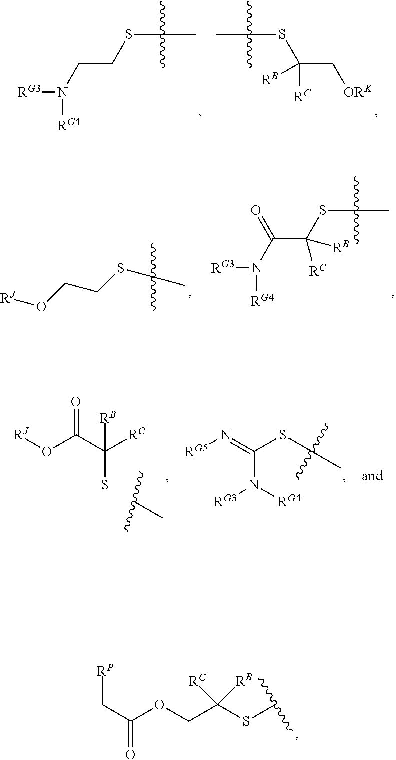

wherein
R^{B} and R^{C} are each independently hydrogen, alkyl, heteroalkyl, cycloalkyl, heterocyclyl, aryl, or heteroaryl, wherein the alkyl, heteroalkyl, cycloalkyl, heterocyclyl, aryl, or heteroaryl is optionally substituted;
R^{G3} and R^{G4} are each independently hydrogen, alkyl, or heteroalkyl, wherein the alkyl or heteroalkyl is optionally substituted;
or R^{G3} and R^{G4}, together with the atoms to which they are attached, form a 3-7 membered cycloalkyl, heterocyclyl, aryl, or heteroaryl, wherein the cycloalkyl, heterocyclyl, aryl, or heteroaryl is optionally substituted;
R^{G5} is hydrogen, alkyl, or heteroalkyl, wherein the alkyl or heteroalkyl is optionally substituted;
R^{J} is hydrogen, alkyl, alkylene-heterocyclyl, heteroalkyl, or heterocyclyl, wherein the alkyl, alkylene, heteroalkyl, or heterocyclyl is optionally substituted;
R^{K} is hydrogen, alkyl, heteroalkyl, cycloalkyl, heterocyclyl, aryl, or heteroaryl, wherein the alkyl, heteroalkyl, cycloalkyl, heterocyclyl, aryl, or heteroaryl is optionally substituted;
R^{P} is halo, —NH-alkyl, —NH-heteroalkyl, —N-(alkyl)₂ (e.g., —N(Me)₂), —N-(heteroalkyl)₂ (e.g., —N(CH₂CH₂OH)₂), or heterocyclyl (e.g., morpholinyl); and
[symbol] denotes the point of attachment of the sulfur atom to the carbon atom of the —C(R^{7A})(R^{7B}) group.

Each optional substituent of a group in Formula (IC) (e.g., an optional substituent on an alkyl, alkylene, alkenyl, alkenylene, alkynyl, alkynylene, heteroalkyl, heteroalkylene, cycloalkyl, cycloalkylene, heterocyclyl, heterocyclylene, aryl, or heteroaryl group) may be any suitable substituent, e.g., a substituent described herein. For example, the optional substituent can be alkyl (e.g., C₁-C₆ alkyl), alkenyl (e.g., C₂-C₆ alkenyl), alkynyl (e.g., C₂-C₆ alkynyl), heteroalkyl (e.g., C₁-C₆ heteroalkyl), haloalkyl (e.g., C₁-C₆ haloalkyl, e.g., —CH₂Cl, —CH₂I, —(CH₂)₂Cl, —(CH₂)₂I, or —CF₃), cycloalkyl (e.g., C₃-C₈ cycloalkyl, e.g., cyclopropyl, cyclobutyl, cyclopentyl, or cyclohexyl), heterocyclyl (e.g., C₃-C₈ heterocyclyl, e.g., morpholinyl, piperazinyl, N-methylpiperazinyl, azetidinyl, or N-Boc-azetidinyl), alkylaryl (e.g., benzyl), aryl (e.g., phenyl), heteroaryl (e.g., pyrrolyl, imidazolyl, or indolyl), halogen (e.g., —F, —Cl, —Br, or —I), hydroxyl, alkylcarbonyloxy, arylcarbonyloxy, alkoxy (e.g., —OMe, —OEt, or —OBn), alkoxycarbonyloxy, aryloxycarbonyloxy, carboxylate, alkylcarbonyl, arylcarbonyl, alkoxycarbonyl, amino (e.g., —NH₂, —NHMe, or —NMe₂), aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl, and ureido), amidino, imino, sulfhydryl, alkylthio, arylthio, thiocarboxylate, sulfate, sulfinyl, sulfonyl, alkylsulfinyl, sulfonato, sulfamoyl, sulfonamido, oxo, nitro, trifluoromethyl, cyano, azido, alkyl-cycloalkyl, alkyl-heterocyclyl, alkylheteroaryl, —C(O)OH, —C(O)O-alkyl (e.g., —C(O)O-tBu or —C(O)OMe), —C(O)O— heteroalkyl, —C(O)O-alkylaryl, —OC(O)O-alkyl, —OC(O)O-heteroalkyl, —OC(O)O-alkylaryl, —C(O)NH₂, —C(O)NH-alkyl, —C(O)NH-heteroalkyl, —C(O)NH-alkylaryl, —NHC(O)O-alkyl (e.g., —NHC(O)OtBu), —NHC(O)O-heteroalkyl, —NHC(O)O-alkylaryl (e.g., —NH-Cbz), —C(O)N(alkyl)₂, —C(O)N(alkyl)(heteroalkyl), —C(O)N(heteroalkyl)₂, —N(alkyl)C(O)O-alkyl (e.g., N(Me)C(O)OtBu), —N(alkyl)C(O)O-heteroalkyl, —N(alkyl)C(O)O-alkylaryl, —N(alkyl)C(O)N(alkyl)₂, —N(alkyl)C(O)N(alkyl)(heteroalkyl), —N(alkyl)C(O)N(heteroalkyl)₂, —N(alkyl)C(O)NH-alkylaryl, ═NH, or ═N-alkyl. Cyclic groups (e.g., cycloalkyl, heterocyclyl, aryl, and heteroaryl) can be substituted at one or more ring positions with any suitable substituent, such as one of the substituents listed above.

The —S—(R⁴) group in Formula (IC) may be a moiety selected from the group consisting of

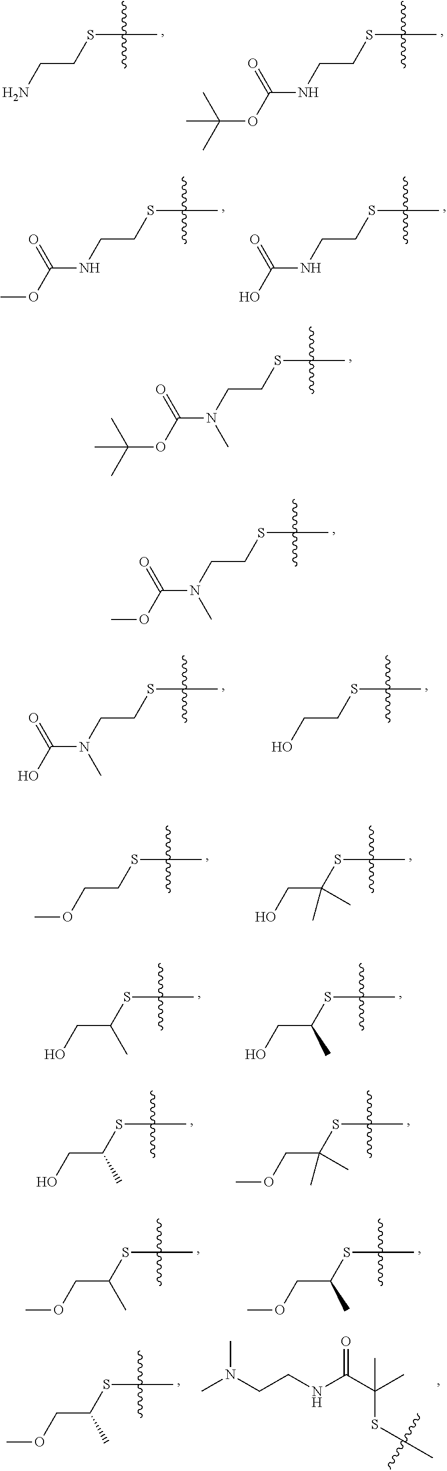

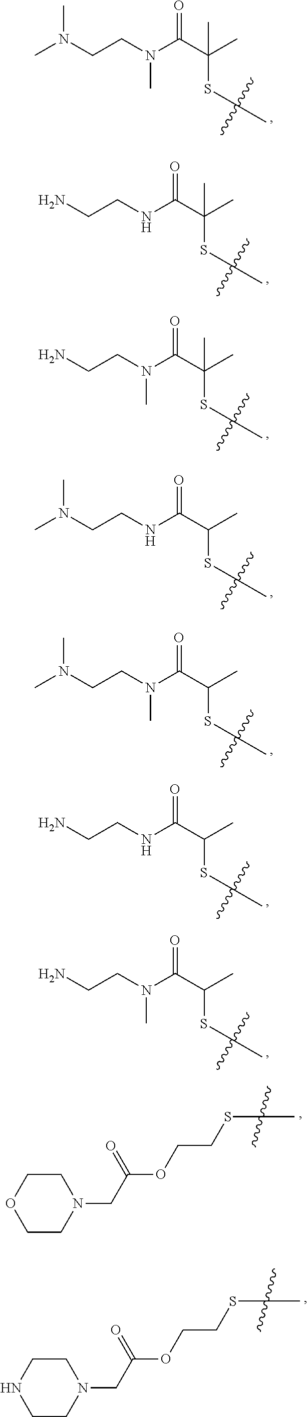

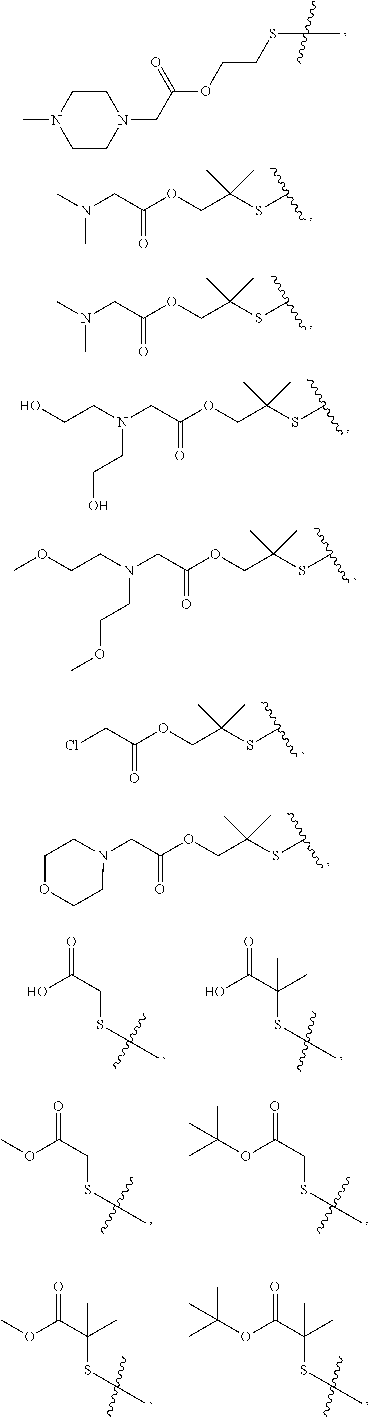

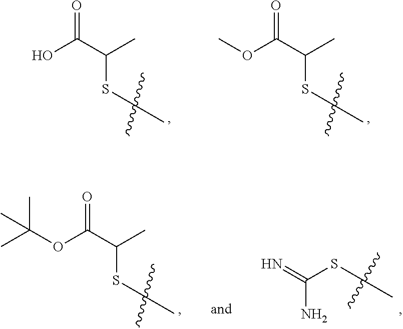

wherein [symbol] denotes the point of attachment of the sulfur atom to the carbon atom of the —C(R^{7A})(R^{7B}) group.

The compound of Formula (I) or Formula (I-I) may be a compound of Formula (ID).

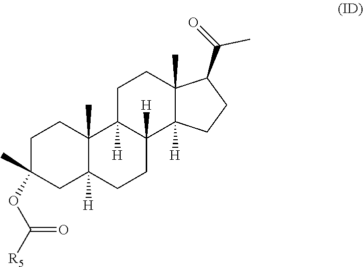

or a pharmaceutically acceptable salt, solvate, hydrate, tautomer, or stereoisomer thereof,
wherein
R⁵ is alkyl, alkenyl, alkynyl, heteroalkyl, haloalkyl, cycloalkyl, heterocyclyl, aryl, heteroaryl, haloalkyl, alkylene-heterocyclyl, alkylene-aryl, alkylene-heteroaryl, heteroalkylene-heterocyclyl, heteroalkylene-aryl, heteroalkylene-heteroaryl, cycloalkylene-alkyl, cycloalkylene-heteroalkyl, heterocyclylene-alkyl, or heterocyclylene-heteroalkyl, wherein the alkyl, alkylene, alkenyl, alkynyl, heteroalkyl, heteroalkylene, cycloalkyl, cycloalkylene, heterocyclyl, heterocyclylene, aryl, or heteroaryl is optionally substituted.

The —R⁵ group in Formula (ID) may be a moiety selected from the group consisting of

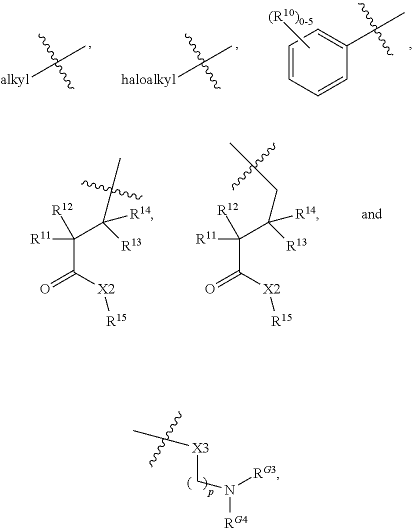

wherein
X2 and X3 are each independently —CH₂—, —O—, —S—, or —N(R^{G3})— (e.g., —NH—);
R¹⁰ is alkyl, alkenyl, alkynyl, haloalkyl, halo, —OR, —N(R^{G3})(R^{G4}); —NO₂, —CN, or —NC;
R¹¹, R¹², R¹³, and R¹⁴ are each independently hydrogen, alkyl, alkenyl, alkynyl, heteroalkyl, haloalkyl, halo, or —N(R^{G3})(R^{G4}), wherein the alkyl, alkenyl, alkynyl, heteroalkyl, or haloalkyl is optionally substituted,
or one of R¹¹ and R¹², and one of R¹³ and R¹⁴, together with the atoms to which they are attached, form a 3-7 membered cycloalkyl, heterocyclyl, aryl, or heteroaryl, wherein the cycloalkyl, heterocyclyl, aryl, or heteroaryl is optionally substituted;
R¹⁵ is hydrogen, alkyl, alkenyl, alkynyl, heteroalkyl, haloalkyl, or halo; wherein the alkyl, alkenyl, alkynyl, heteroalkyl, or haloalkyl is optionally substituted;
R^{G3} and R^{G4} are each independently hydrogen, alkyl, or heteroalkyl, wherein the alkyl or heteroalkyl is optionally substituted;
or R^{G3} and R^{G4}, together with the atoms to which they are attached, form a 3-7 membered cycloalkyl, heterocyclyl, aryl, or heteroaryl, wherein the cycloalkyl, heterocyclyl, aryl, or heteroaryl is optionally substituted;
R^{J} is hydrogen, alkyl, alkylene-heterocyclyl, heteroalkyl, or heterocyclyl, wherein the alkyl, alkylene, heteroalkyl, or heterocyclyl is optionally substituted; and
p is an integer between 0 and 4; and
[symbol] denotes the point of attachment of the R⁵ group to the carbon atom of the —C(O)— group.

Each optional substituent of a group in Formula (ID) (e.g., an optional substituent on an alkyl, alkylene, alkenyl, alkenylene, alkynyl, alkynylene, heteroalkyl, heteroalkylene, cycloalkyl, cycloalkylene, heterocyclyl, heterocyclylene, aryl, or heteroaryl group) may be any suitable substituent, e.g., a substituent described herein. For example, the optional substituent can be alkyl (e.g., C₁-C₆ alkyl), alkenyl (e.g., C₂-C₆ alkenyl), alkynyl (e.g., C₂-C₆ alkynyl), heteroalkyl (e.g., C₁-C₆ heteroalkyl), haloalkyl (e.g., C₁-C₆ haloalkyl, e.g., —CH₂Cl, —CH₂I, —(CH₂)₂Cl, —(CH₂)₂I, or —CF₃), cycloalkyl (e.g., C₃-C₈ cycloalkyl, e.g., cyclopropyl, cyclobutyl, cyclopentyl, or cyclohexyl), heterocyclyl (e.g., C₃-C₈ heterocyclyl, e.g., morpholinyl, piperazinyl, N-methylpiperazinyl, azetidinyl, or N-Boc-azetidinyl), alkylaryl (e.g., benzyl), aryl (e.g., phenyl), heteroaryl (e.g., pyrrolyl, imidazolyl, or indolyl), halogen (e.g., —F, —Cl, —Br, or —I), hydroxyl, alkylcarbonyloxy, arylcarbonyloxy, alkoxy (e.g., —OMe, —OEt, or —OBn), alkoxycarbonyloxy, aryloxycarbonyloxy, carboxylate, alkylcarbonyl, arylcarbonyl, alkoxycarbonyl, amino (e.g., —NH₂, —NHMe, or —NMe₂), aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl, and ureido), amidino, imino, sulfhydryl, alkylthio, arylthio, thiocarboxylate, sulfate, sulfinyl, sulfonyl, alkylsulfinyl, sulfonato, sulfamoyl, sulfonamido, oxo, nitro, trifluoromethyl, cyano, azido, alkyl-cycloalkyl, alkyl-heterocyclyl, alkylheteroaryl, —C(O)OH, —C(O)O-alkyl (e.g., —C(O)O-tBu or —C(O)OMe), —C(O)O— heteroalkyl, —C(O)O-alkylaryl, —OC(O)O-alkyl, —OC(O)O-heteroalkyl, —OC(O)O-alkylaryl, —C(O)NH₂, —C(O)NH-alkyl, —C(O)NH-heteroalkyl, —C(O)NH-alkylaryl, —NHC(O)O-alkyl (e.g., —NHC(O)OtBu), —NHC(O)O-heteroalkyl, —NHC(O)O-alkylaryl (e.g., —NH-Cbz), —C(O)N(alkyl)₂, —C(O)N(alkyl)(heteroalkyl), —C(O)N(heteroalkyl)₂, —N(alkyl)C(O)O-alkyl (e.g., N(Me)C(O)OtBu), —N(alkyl)C(O)O-heteroalkyl, —N(alkyl)C(O)O-alkylaryl, —N(alkyl)C(O)N(alkyl)₂, —N(alkyl)C(O)N(alkyl)(heteroalkyl), —N(alkyl)C(O)N(heteroalkyl)₂, —N(alkyl)C(O)NH-alkylaryl, ═NH, or ═N-alkyl. Cyclic groups (e.g., cycloalkyl, heterocyclyl, aryl, and heteroaryl) can be substituted at one or more ring positions with any suitable substituent, such as one of the substituents listed above.

The —R⁵ group in Formula (ID) may be a moiety selected from the group consisting of

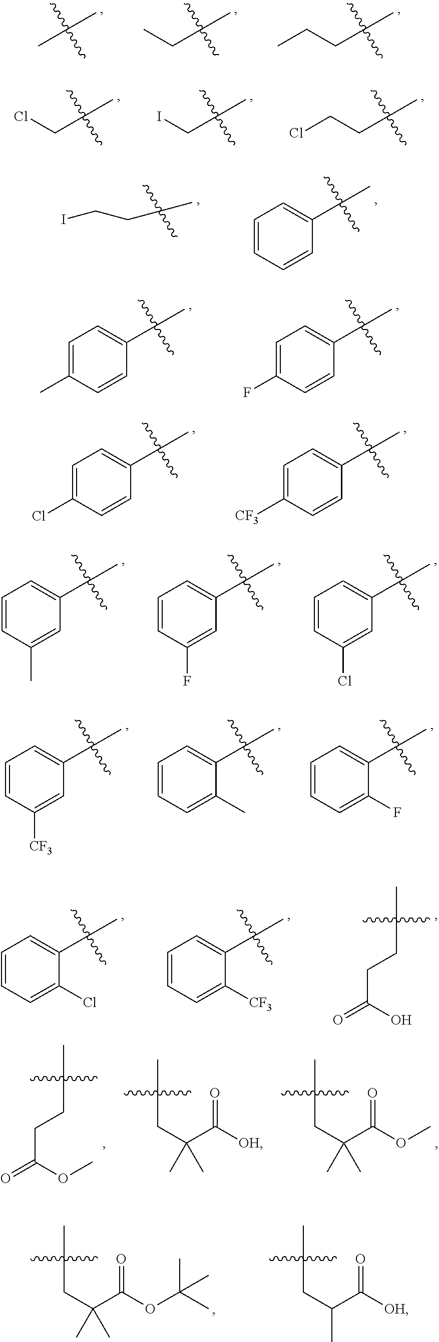

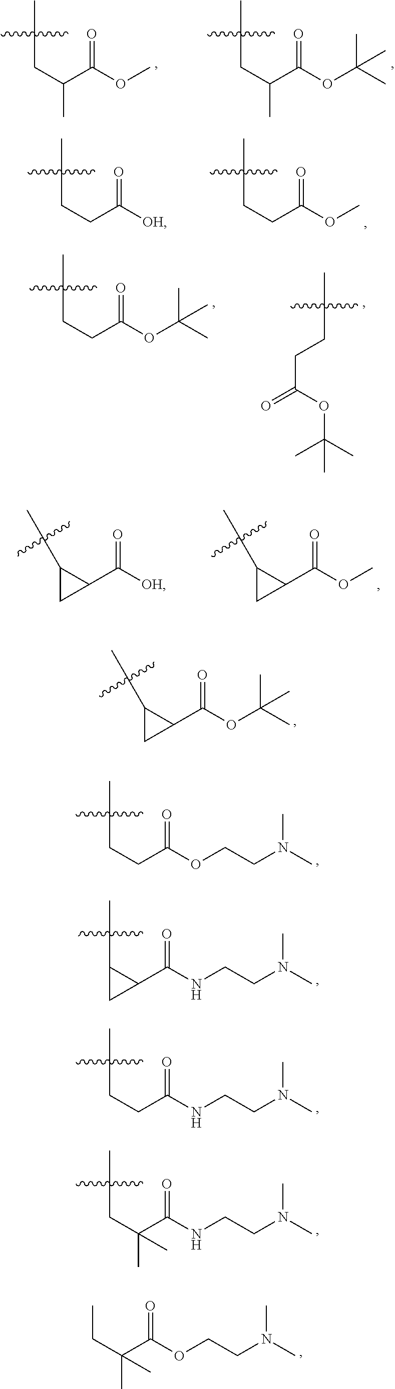

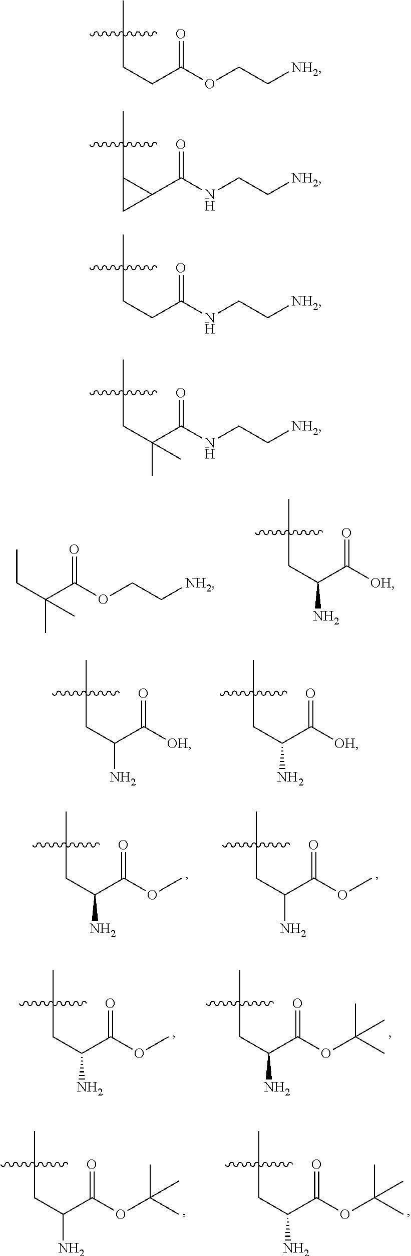

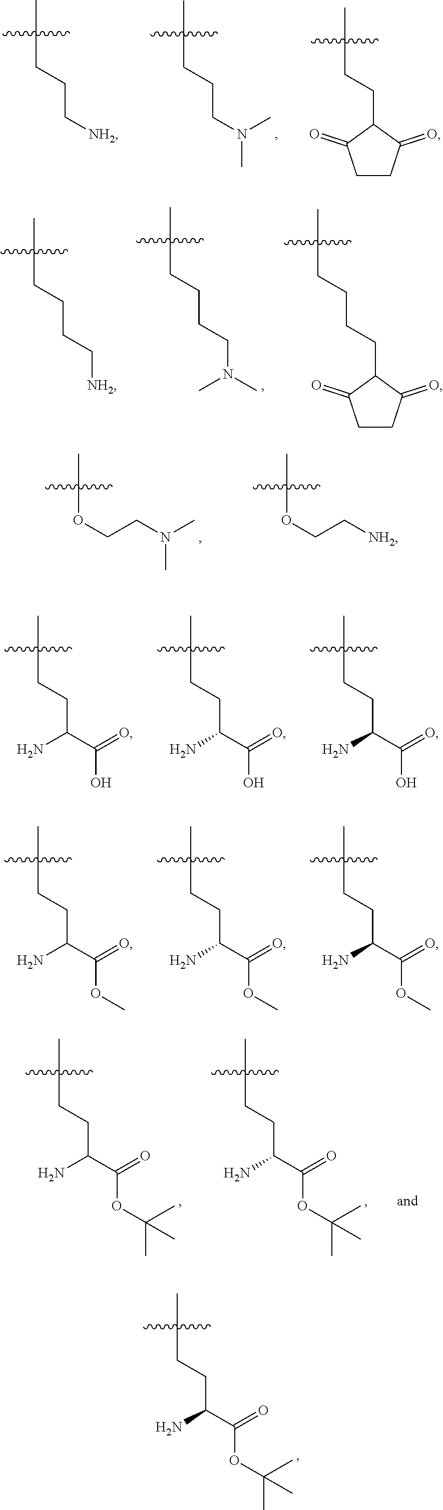

wherein [symbol] denotes the point of attachment of the R⁵ group to the carbon atom of the —C(O)— group.

The compound of Formula (I) or Formula (I-I) may be a compound of Formula (IE):

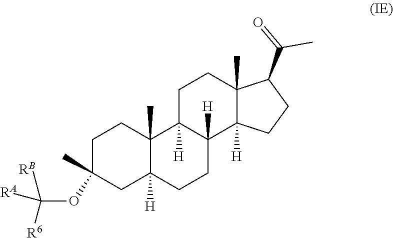

or a pharmaceutically acceptable salt, solvate, hydrate, tautomer, or stereoisomer thereof, wherein
R⁶ is alkyl, alkenyl, alkynyl, heteroalkyl, cycloalkyl, heterocyclyl, aryl, heteroaryl, haloalkyl, alkylene-cycloalkyl, alkylene-heterocyclyl, alkylene-aryl, alkylene-heteroaryl, heteroalkylene-cycloalkyl, heteroalkylene-heterocyclyl, heteroalkylene-aryl, heteroalkylene-heteroaryl, —OC(O)-alkyl, —C(O)O-alkyl, —OC(O)-heteroalkyl, —C(O)O-heteroalkyl, —N₃, —SH, or —S-alkyl (e.g., —S-Me), wherein the alkyl, alkylene, alkenyl, alkynyl, heteroalkyl, heteroalkylene, cycloalkyl, heterocyclyl, aryl, or heteroaryl is optionally substituted; and
R^{A} and R^{B} are each independently hydrogen, alkyl, heteroalkyl, cycloalkyl, heterocyclyl, aryl, or heteroaryl, wherein the alkyl, heteroalkyl, cycloalkyl, heterocyclyl, aryl, or heteroaryl is optionally substituted.

The R⁶ group in Formula (IE) may be a moiety selected from the group consisting of

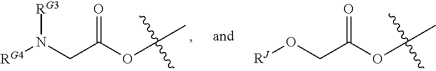

wherein
R^{G3} and R^{G4} are each independently hydrogen, alkyl, or heteroalkyl, wherein the alkyl or heteroalkyl is optionally substituted;
or R^{G3} and R^{G4}, together with the atoms to which they are attached, form a 3-7 membered cycloalkyl, heterocyclyl, aryl, or heteroaryl, wherein the cycloalkyl, heterocyclyl, aryl, or heteroaryl is optionally substituted;
R^{J} is hydrogen, alkyl, alkylene-heterocyclyl, heteroalkyl, or heterocyclyl, wherein the alkyl, alkylene, heteroalkyl, or heterocyclyl is optionally substituted;
and [symbol] denotes the point of attachment of the R⁶ group to the carbon atom of the —C(R^{A})(R^{B})— group.

Each optional substituent of a group in Formula (IE) (e.g., an optional substituent on an alkyl, alkylene, alkenyl, alkenylene, alkynyl, alkynylene, heteroalkyl, heteroalkylene, cycloalkyl, cycloalkylene, heterocyclyl, heterocyclylene, aryl, or heteroaryl group) may be any suitable substituent, e.g., a substituent described herein. For example, the optional substituent can be alkyl (e.g., C₁-C₆ alkyl), alkenyl (e.g., C₂-C₆ alkenyl), alkynyl (e.g., C₂-C₆ alkynyl), heteroalkyl (e.g., C₁-C₆ heteroalkyl), haloalkyl (e.g., C₁-C₆ haloalkyl, e.g., —CH₂Cl, —CH₂I, —(CH₂)₂Cl, —(CH₂)₂I, or —CF₃), cycloalkyl (e.g., C₃-C₈ cycloalkyl, e.g., cyclopropyl, cyclobutyl, cyclopentyl, or cyclohexyl), heterocyclyl (e.g., C₃-C₈ heterocyclyl, e.g., morpholinyl, piperazinyl, N-methylpiperazinyl, azetidinyl, or N-Boc-azetidinyl), alkylaryl (e.g., benzyl), aryl (e.g., phenyl), heteroaryl (e.g., pyrrolyl, imidazolyl, or indolyl), halogen (e.g., —F, —Cl, —Br, or —I), hydroxyl, alkylcarbonyloxy, arylcarbonyloxy, alkoxy (e.g., —OMe, —OEt, or —OBn), alkoxycarbonyloxy, aryloxycarbonyloxy, carboxylate, alkylcarbonyl, arylcarbonyl, alkoxycarbonyl, amino (e.g., —NH₂, —NHMe, or —NMe₂), aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl, and ureido), amidino, imino, sulfhydryl, alkylthio, arylthio, thiocarboxylate, sulfate, sulfinyl, sulfonyl, alkylsulfinyl, sulfonato, sulfamoyl, sulfonamido, oxo, nitro, trifluoromethyl, cyano, azido, alkyl-cycloalkyl, alkyl-heterocyclyl, alkylheteroaryl, —C(O)OH, —C(O)O-alkyl (e.g., —C(O)O-tBu or —C(O)OMe), —C(O)O— heteroalkyl, —C(O)O-alkylaryl, —OC(O)O-alkyl, —OC(O)O-heteroalkyl, —OC(O)O-alkylaryl, —C(O)NH₂, —C(O)NH-alkyl, —C(O)NH-heteroalkyl, —C(O)NH-alkylaryl, —NHC(O)O-alkyl (e.g., —NHC(O)OtBu), —NHC(O)O-heteroalkyl, —NHC(O)O-alkylaryl (e.g., —NH-Cbz), —C(O)N(alkyl)₂, —C(O)N(alkyl)(heteroalkyl), —C(O)N(heteroalkyl)₂, —N(alkyl)C(O)O-alkyl (e.g., N(Me)C(O)OtBu), —N(alkyl)C(O)O-heteroalkyl, —N(alkyl)C(O)O-alkylaryl, —N(alkyl)C(O)N(alkyl)₂, —N(alkyl)C(O)N(alkyl)(heteroalkyl), —N(alkyl)C(O)N(heteroalkyl)₂, —N(alkyl)C(O)NH-alkylaryl, ═NH, or ═N-alkyl. Cyclic groups (e.g., cycloalkyl, heterocyclyl, aryl, and heteroaryl) can be substituted at one or more ring positions with any suitable substituent, such as one of the substituents listed above.

The R⁶ group in Formula (IE) may be a moiety selected from the group consisting of

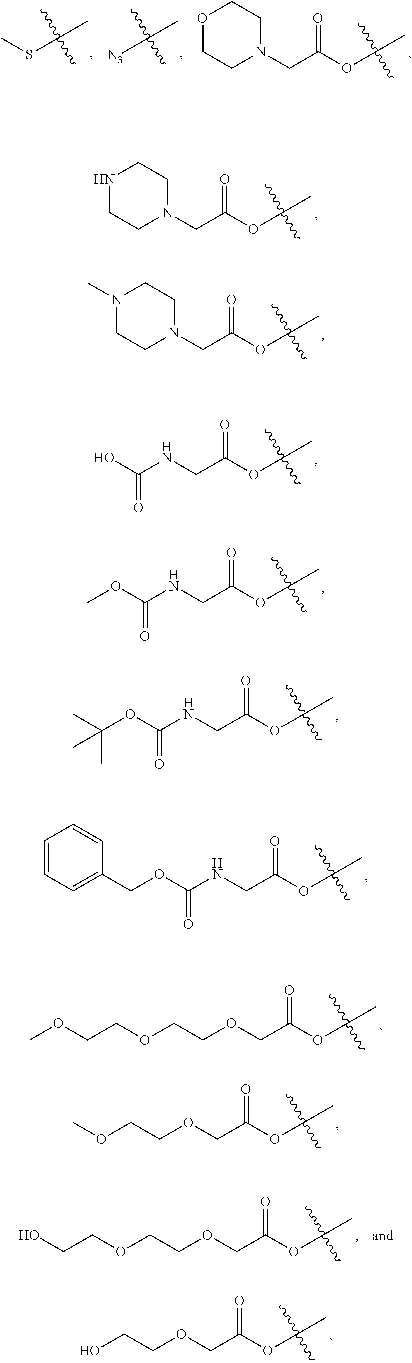

wherein [symbol] denotes the point of attachment of the R⁶ group to the carbon atom of the —C(R^{A})(R^{B})— group.

A compound of the present disclosure may be a compound of Formula (I-II):

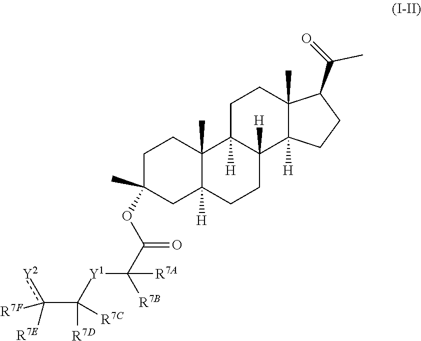

or a pharmaceutically acceptable salt, solvate, hydrate, tautomer, or stereoisomer thereof, wherein:
[symbol] denotes a single bond or a double bond;
Y¹ is alkylene (e.g., methylene), alkenylene, alkynylene, heteroalkylene, —C(O)—, —OC(O)—, —C(O)O—, —NR¹—, —O—, or —S—, wherein the alkylene, alkenylene, alkynylene, or heteroalkylene is optionally substituted;
Y² is heteroalkyl, heterocyclyl, heteroaryl, heteroalkylene-cycloalkyl, heteroalkylene-heterocyclyl, heteroalkylene-aryl, heteroalkylene-heteroaryl, —NR¹R², —OR³, —SR⁴, ═NR¹, or ═O, wherein the heteroalkyl, cycloalkyl, heterocyclyl, aryl, or heteroaryl is optionally substituted;
R¹ and R² are each independently hydrogen, alkyl, alkenyl, alkynyl, heteroalkyl, cycloalkyl, heterocyclyl, aryl, heteroaryl, alkylene-cycloalkyl, alkylene-heterocyclyl, alkylene-aryl, alkylene-heteroaryl, heteroalkylene-cycloalkyl, heteroalkylene-heterocyclyl, heteroalkylene-aryl, heteroalkylene-heteroaryl, cycloalkylene-alkyl, cycloalkylene-heteroalkyl, heterocyclylene-alkyl, or heterocyclylene-heteroalkyl, wherein the alkyl, alkylene, alkenyl, alkynyl, heteroalkyl, heteroalkylene, cycloalkyl, cycloalkylene, heterocyclyl, heterocyclylene, aryl, or heteroaryl is optionally substituted;
or R¹ and R², together with the atoms to which they are attached, form a 3-7 membered cycloalkyl, heterocyclyl, aryl, or heteroaryl, wherein the cycloalkyl, heterocyclyl, aryl, or heteroaryl is optionally substituted;
R³ is hydrogen, alkyl, alkenyl, alkynyl, heteroalkyl, cycloalkyl, heterocyclyl, aryl, heteroaryl, alkylene-cycloalkyl, alkylene-heterocyclyl, alkylene-aryl, alkylene-heteroaryl, heteroalkylene-cycloalkyl, heteroalkylene-heterocyclyl, heteroalkylene-aryl, heteroalkylene-heteroaryl, cycloalkylene-alkyl, cycloalkylene-heteroalkyl, heterocyclylene-alkyl, or heterocyclylene-heteroalkyl, wherein the alkyl, alkylene, alkenyl, alkynyl, heteroalkyl, heteroalkylene, cycloalkyl, cycloalkylene, heterocyclyl, heterocyclylene, aryl, or heteroaryl is optionally substituted;
R⁴ is hydrogen, alkyl, alkenyl, alkynyl, heteroalkyl, cycloalkyl, heterocyclyl, aryl, heteroaryl, alkylene-cycloalkyl, alkylene-heterocyclyl, alkylene-aryl, alkylene-heteroaryl, heteroalkylene-cycloalkyl, heteroalkylene-heterocyclyl, heteroalkylene-aryl, heteroalkylene-heteroaryl, wherein the alkyl, alkylene, alkenyl, alkynyl, heteroalkyl, heteroalkylene, cycloalkyl, cycloalkylene, heterocyclyl, heterocyclylene, aryl, or heteroaryl is optionally substituted;
R^{7A} and R^{7B} are each independently hydrogen or optionally substituted alkyl, or one of R^{7A} and R^{7B} is bonded to Y¹ through one or more carbon atoms or heteroatoms to form a cyclic moiety (e.g., cycloalkyl, heterocyclyl, aryl, or heteroaryl, e.g., pyrrole), and the other of the R^{7A} and R^{7B} groups is hydrogen or optionally substituted alkyl;
R^{7C} and R^{7D} are each independently hydrogen or optionally substituted alkyl, or R^{7C} and R^{7D} are each bonded to the same oxygen atom to form an oxo group; and
R^{7E} and R^{7F} are each independently absent, hydrogen, alkyl, heteroalkyl, —NR¹R², —OR³, or —SR⁴, wherein the alkyl or heteroalkyl is optionally substituted, with the proviso that when [symbol] is a double bond, one of R^{7E} and R^{7F} is absent, or R^{7E} and R^{7F} are each bonded to the same oxygen to form an oxo group.

In some embodiments Y¹ of Formula (I-II) is —O— or —N(R¹)— (e.g., —N(H)—, —N(alkyl)-, or —N(heteroalkyl)-). For example, Y¹ of Formula (I-II) may be —O— or —N(H)—. Alternatively, Y¹ of Formula (I-II) may be —N(Me)—, —N(Et)-, —N(EtOH)—, or —N(ⁿPrOH)—.

The compound of Formula (I-II) may be a compound of Formula (I-IIa):

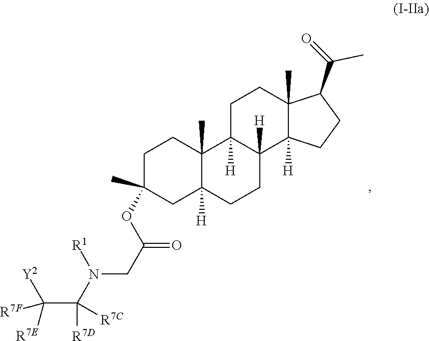

or a pharmaceutically acceptable salt, solvate, hydrate, tautomer, or stereoisomer thereof, wherein Y², R¹, R^{7C}, R^{7D}, R^{7E}, and R^{7F} are as defined above.

The compound of Formula (I-II) may be a compound of Formula (I-IIb):

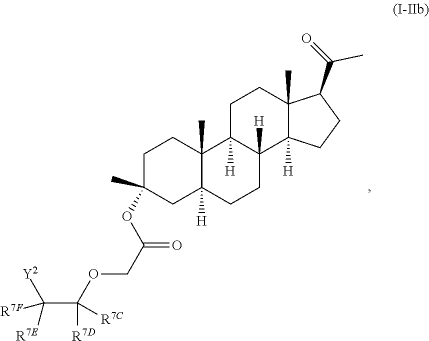

or a pharmaceutically acceptable salt, solvate, hydrate, tautomer, or stereoisomer thereof, wherein Y², R^{7C}, R^{7D}, R^{7E}, and R^{7F} are as defined above.

The compound of Formula (I-II) may be a compound of Formula (I-IIc):

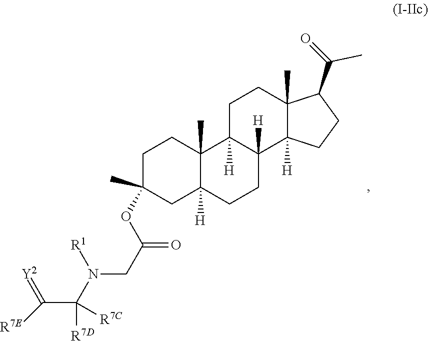

or a pharmaceutically acceptable salt, solvate, hydrate, tautomer, or stereoisomer thereof, wherein Y², R¹, R^{7C}, R^{7D}, and R^{7E} are as defined above.

The compound of Formula (I-II) may be a compound of Formula (I-IId):

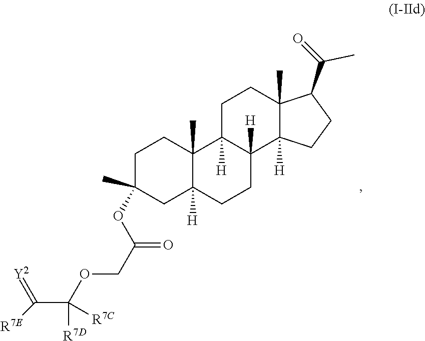

or a pharmaceutically acceptable salt, solvate, hydrate, tautomer, or stereoisomer thereof, wherein Y², R^{7C}, R^{7D}, and R^{7E} are as defined above.

The compound of Formula (I-II) may be a compound of Formula (I-IIe):

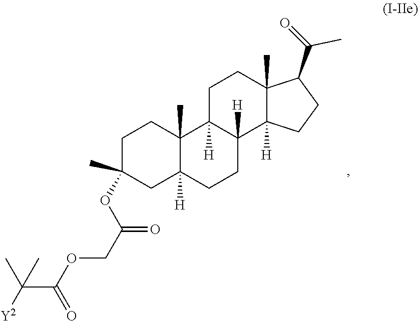

or a pharmaceutically acceptable salt, solvate, hydrate, tautomer, or stereoisomer thereof, wherein Y² is as defined above.

Y² of Formulae (I-II), (I-IIa), (I-IIb), (I-IIc), (I-IId), or (I-IIe), or R^{7E} when [symbol] is a double bond, may be optionally substituted heteroalkyl, —NR¹R²(e.g., —NH₂, —NHMe, or —NMe₂), —OR³ (e.g., —OH, —OMe, —OEt, or —OtBu), or ═O. For example, Y² of Formulae (I-II), (I-IIa), (I-IIb), (I-IIc), (I-IId), or (I-IIe), or R^{7E} when [symbol] is a double bond, may be —NH₂, —NHMe, or —OH.

Alternatively, Y² of Formulae (I-II), (I-IIa), (I-IIb), (I-IIc), (I-IId), or (I-IIe), or R^{7E} when [symbol] is a double bond, may be optionally substituted heteroalkyl, for example a substituted or unsubstituted heteroalkyl selected from the group consisting of:

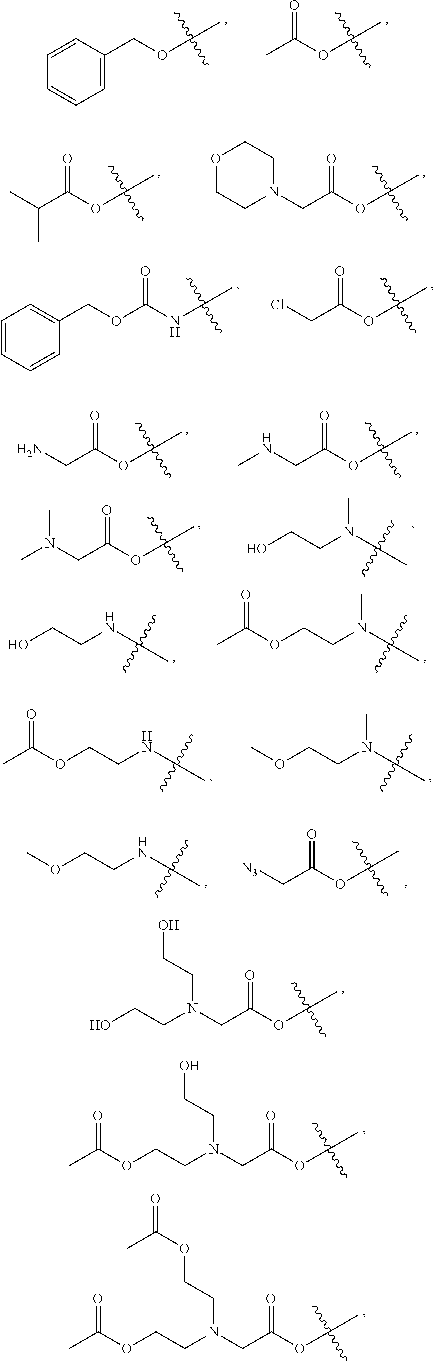

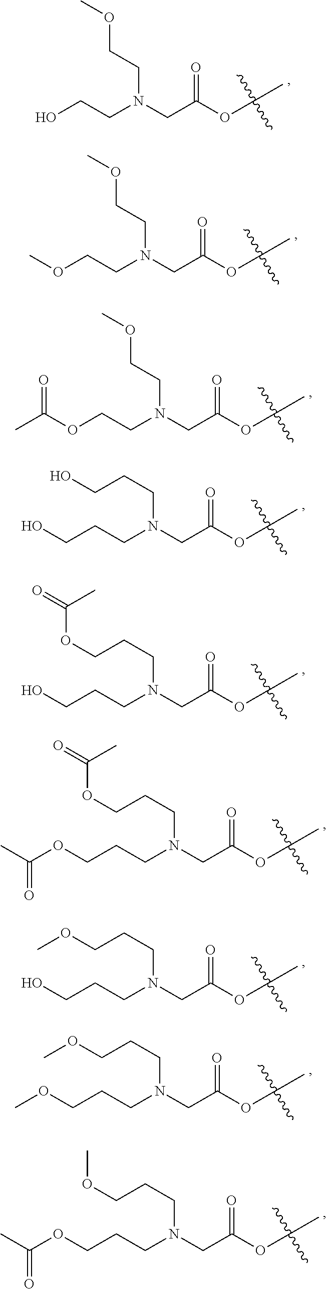

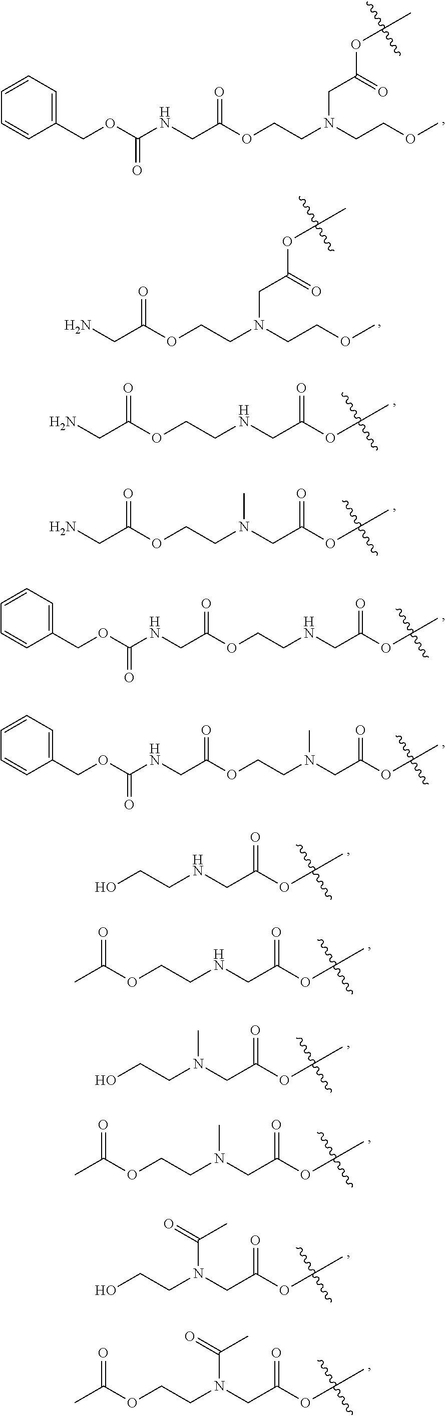

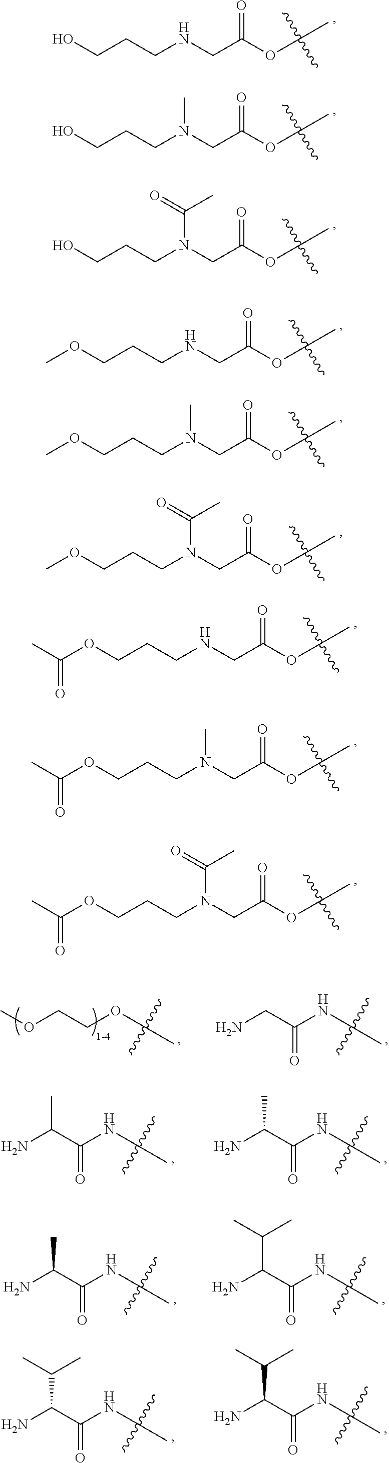

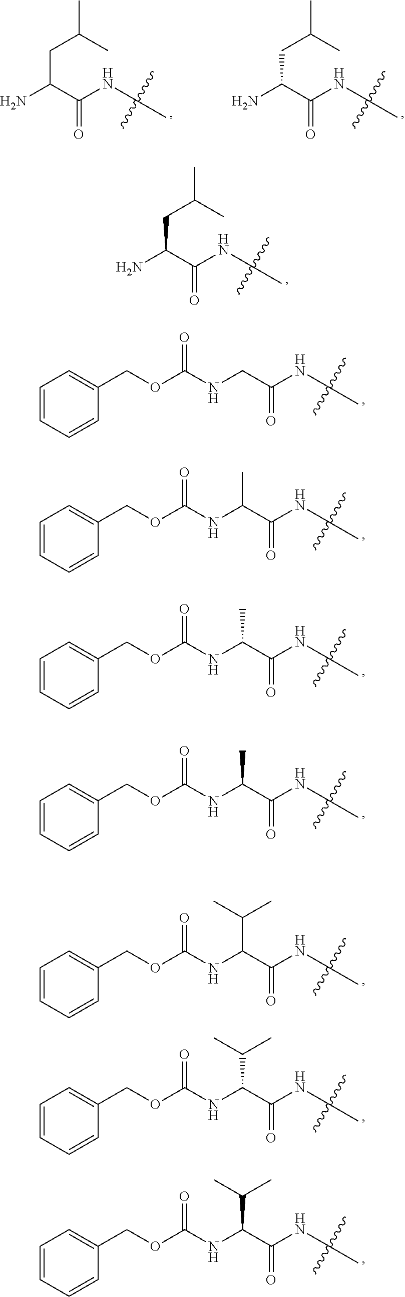

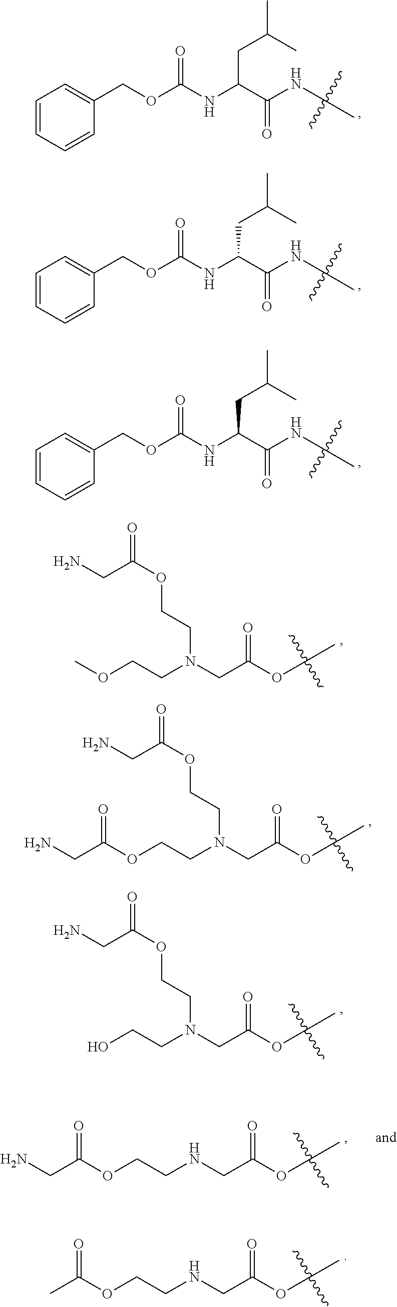

For example, Y² of Formulae (I-II), (I-IIa), (I-IIb), (I-IIc), (I-IId), or (I-IIe) may be selected from the group consisting of: —OH, —NH₂, —NHMe, —NMe₂,

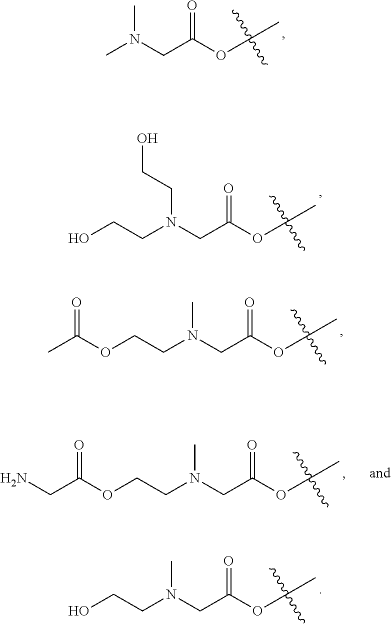

R^{7A} and R^{7B} of Formula (I-II) may each be independently hydrogen.

R^{7C} and R^{7D} of Formulae (I-II), (I-IIa), (I-IIb), (I-IIc), or (I-IId) may each be independently hydrogen or methyl, or R^{7C} and R^{7D} of Formulae (I-II), (I-IIa), (I-IIb), (I-IIc), or (I-IId) may each be bonded to the same oxygen atom to form an oxo group.

R^{7E} and R^{7F} of Formulae (I-II), (I-IIa), (I-IIb), (I-IIc), or (I-IId) may each be independently hydrogen or methyl, or R^{7C} and R^{7D} of Formulae (I-II), (I-IIa), (I-IIb), (I-IIc), or (I-IId) may each be bonded to the same oxygen atom to form an oxo group.

R¹ of Formulae (I-II), (I-IIa), and (I-IIc) may be hydrogen, alkyl, or heteroalkyl. For example, R¹ of Formulae (I-II), (I-IIa), and (I-IIc) may be hydrogen, methyl, ethyl, —MeOH, —EtOH, or —ⁿPrOH.

Each optional substituent of a group in one of Formulae (I-II), (I-IIa), (I-IIb), (I-IIc), (I-IId), or (I-IIe), such as optional substituent on an alkyl, alkylene, alkenyl, alkenylene, alkynyl, alkynylene, heteroalkyl, heteroalkylene, cycloalkyl, cycloalkylene, heterocyclyl, heterocyclylene, aryl, or heteroaryl group) may be any suitable substituent, e.g., a substituent described herein. For example, the optional substituent can be alkyl (e.g., C₁-C₆ alkyl), alkenyl (e.g., C₂-C₆ alkenyl), alkynyl (e.g., C₂-C₆ alkynyl), heteroalkyl (e.g., C₁-C₆ heteroalkyl), haloalkyl (e.g., C₁-C₆ haloalkyl, e.g., —CH₂Cl, —CH₂I, —(CH₂)₂Cl, —(CH₂)₂I, or —CF₃), cycloalkyl (e.g., C₃-C₈ cycloalkyl, e.g., cyclopropyl, cyclobutyl, cyclopentyl, or cyclohexyl), heterocyclyl (e.g., C₃-C₈ heterocyclyl, e.g., morpholinyl, piperazinyl, N-methylpiperazinyl, azetidinyl, or N-Boc-azetidinyl), alkylaryl (e.g., benzyl), aryl (e.g., phenyl), heteroaryl (e.g., pyrrolyl, imidazolyl, or indolyl), halogen (e.g., —F, —Cl, —Br, or —I), hydroxyl, alkylcarbonyloxy, arylcarbonyloxy, alkoxy (e.g., —OMe, —OEt, or —OBn), alkoxycarbonyloxy, aryloxycarbonyloxy, carboxylate, alkylcarbonyl, arylcarbonyl, alkoxycarbonyl, amino (e.g., —NH₂, —NHMe, or —NMe₂), aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl, and ureido), amidino, imino, sulfhydryl, alkylthio, arylthio, thiocarboxylate, sulfate, sulfinyl, sulfonyl, alkylsulfinyl, sulfonato, sulfamoyl, sulfonamido, oxo, nitro, trifluoromethyl, cyano, azido, alkyl-cycloalkyl, alkyl-heterocyclyl, alkylheteroaryl, —C(O)OH, —C(O)O-alkyl (e.g., —C(O)O-tBu or —C(O)OMe), —C(O)O-heteroalkyl, —C(O)O-alkylaryl, —OC(O)O-alkyl, —OC(O)O-heteroalkyl, —OC(O)O-alkylaryl, —C(O)NH₂, —C(O)NH-alkyl, —C(O)NH-heteroalkyl, —C(O)NH-alkylaryl, —NHC(O)O-alkyl (e.g., —NHC(O)OtBu), —NHC(O)O-heteroalkyl, —NHC(O)O-alkylaryl (e.g., —NH-Cbz), —C(O)N(alkyl)₂, —C(O)N(alkyl)(heteroalkyl), —C(O)N(heteroalkyl)₂, —N(alkyl)C(O)O-alkyl (e.g., N(Me)C(O)OtBu), —N(alkyl)C(O)O— heteroalkyl, —N(alkyl)C(O)O-alkylaryl, —N(alkyl)C(O)N(alkyl)₂, —N(alkyl)C(O)N(alkyl)(heteroalkyl), —N(alkyl)C(O)N(heteroalkyl)₂, —N(alkyl)C(O)NH-alkylaryl, ═NH, or ═N-alkyl. Cyclic groups (e.g., cycloalkyl, heterocyclyl, aryl, and heteroaryl) can be substituted at one or more ring positions with any suitable substituent, such as one of the substituents listed above.

A compound disclosed herein, e.g., a compound of Formulae (I), (I-I), (IA), (IA-a), (IA-b), (IB), (IC), (ID), (IE), (I-II), (I-IIa), (I-IIb), (I-IIc), (I-IId), or (I-IIe) may have a structure provided in Table 1, or a pharmaceutically acceptable salt, solvate, hydrate, tautomer, or stereoisomer thereof. In some embodiments, a compound disclosed herein (e.g., a compound of Formulae (I), (I-I), (IA), (IA-a), (IA-b), (IB), (IC), (ID), (IE), (I-II), (I-IIa), (I-IIb), (I-IIc), (I-IId), or (I-IIe) is a hydrochloride (HCl) salt. In some embodiments, a compound disclosed herein is a hydrochloride (HCl) salt of a compound provided in Table 1. In some embodiments, a compound disclosed herein (e.g., a compound of Formulae (I), (I-I), (IA), (IA-a), (IA-b), (IB), (IC), (ID), (IE), (I-II), (I-IIa), (I-IIb), (I-IIc), (I-IId), or (I-IIe) is a trifluoroacetate (TFA) salt. In some embodiments, a compound disclosed herein is a trifluoroacetate (TFA) salt of a compound provided in Table 1.

It will be appreciated that certain compounds may form a salt with more than one counterion, for example, two chloride ions to form a bis-HCl salt. Therefore, it will be appreciated that reference to an HCl salt herein may refer to a monohydrochloride salt or a bis-hydrochloride salt. Similarly, it will be appreciated that reference to a TFA salt herein may refer to a monotrifluoroacetate salt, or a bis-trifluoroacetate salt.

TABLE 1
Exemplary compounds.

| Compound | |
|---|---|
| No. | Structure |
| 100 | 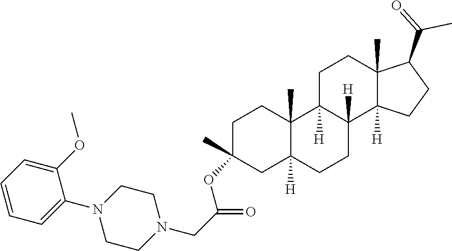 |
| 101 | 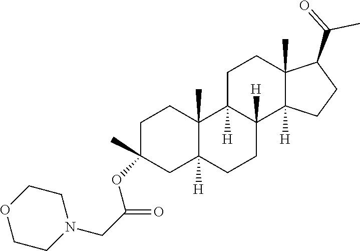 |
| 102 | 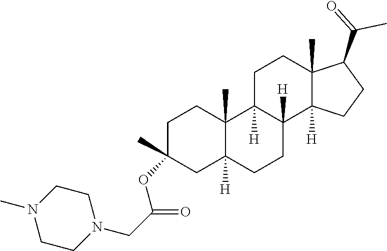 |
| 103 | 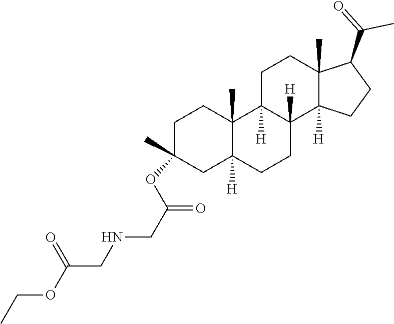 |
| 104 | 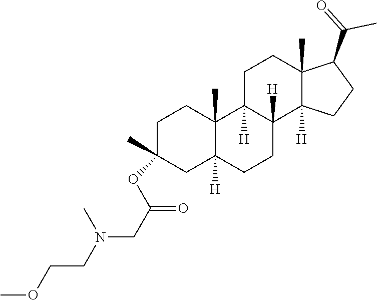 |
| 105 | 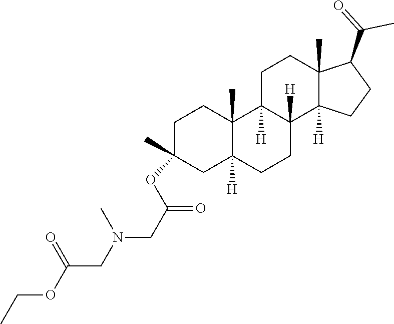 |
| 106 | 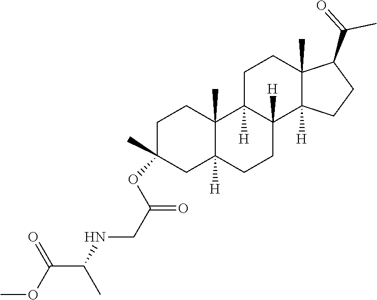 |
| 107 | 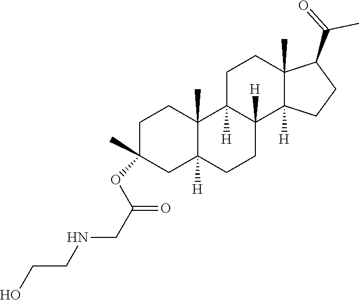 |
| 108 | 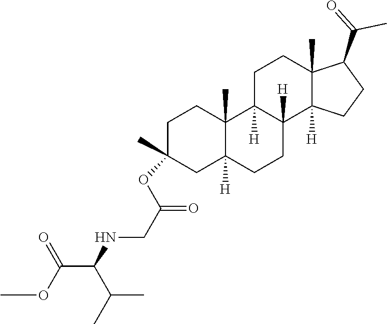 |
| 109 | 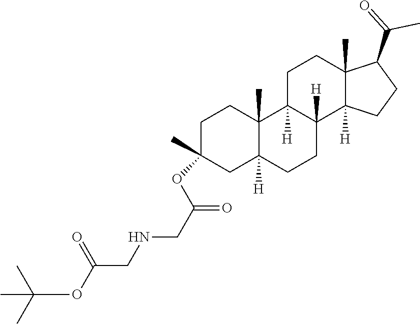 |
| 110 | 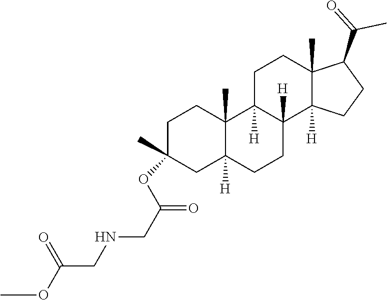 |
| 111 | 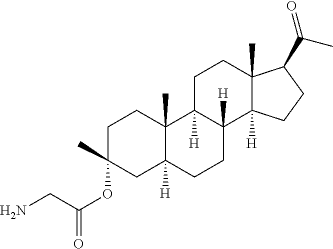 |
| 112 | 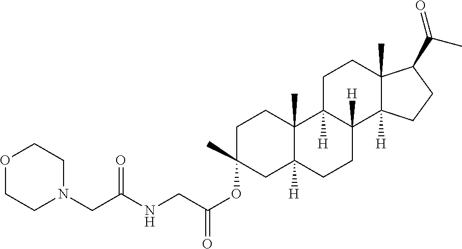 |
| 113 | 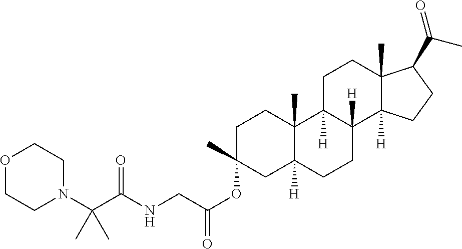 |
| 114 | 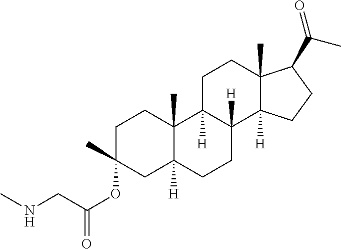 |
| 115 | 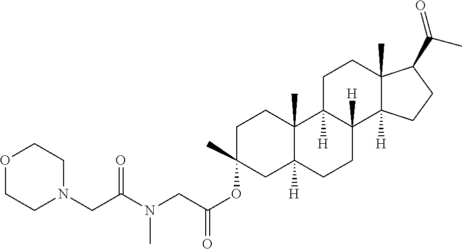 |
| 116 | 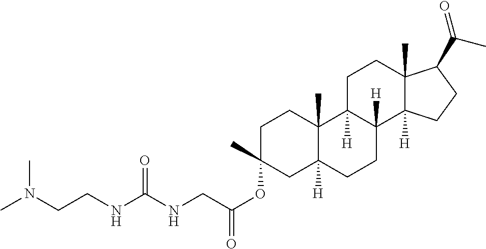 |
| 117 | 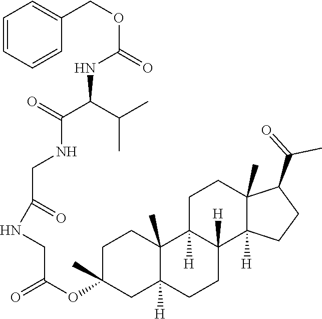 |
| 118 | 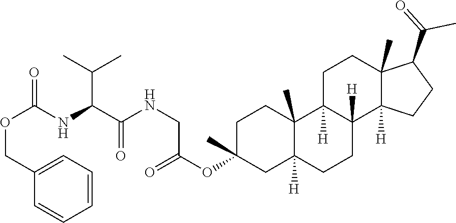 |
| 119 | 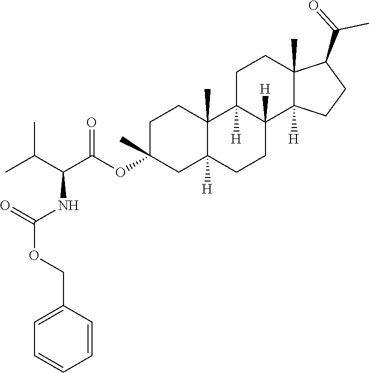 |
| 120 | 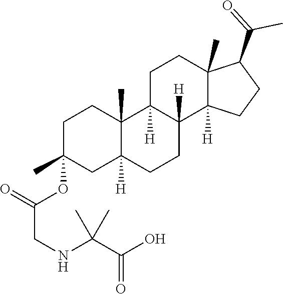 |
| 121 | 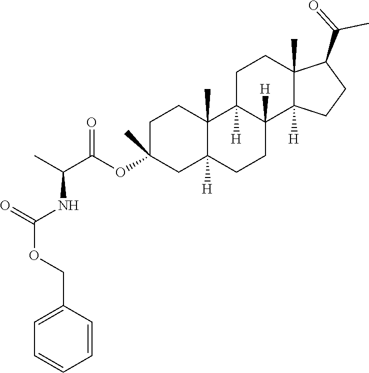 |
| 122 | 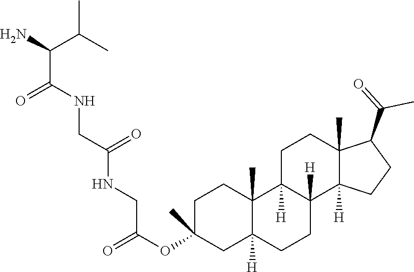 |
| 123 | 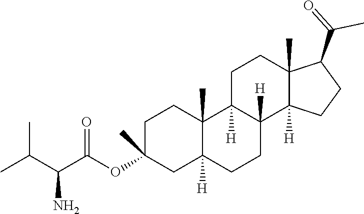 |
| 124 | 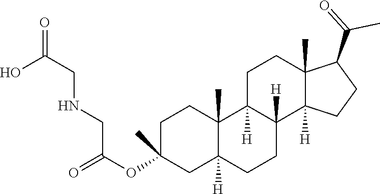 |
| 125 | 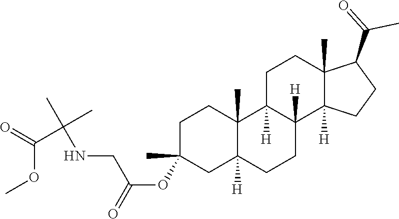 |
| 126 | 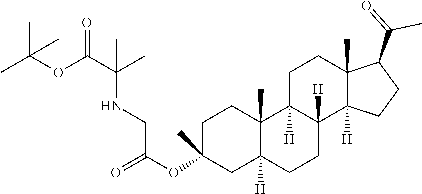 |
| 127 | 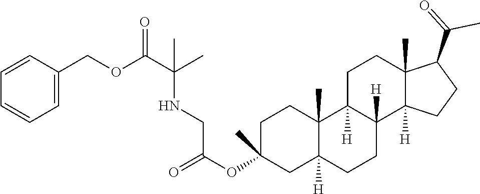 |
| 128 | 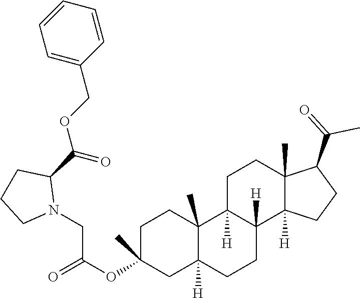 |
| 129 | 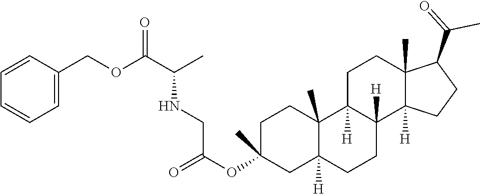 |
| 130 | 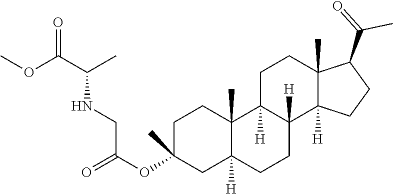 |
| 131 | 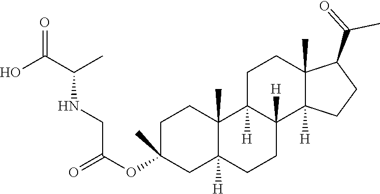 |
| 132 | 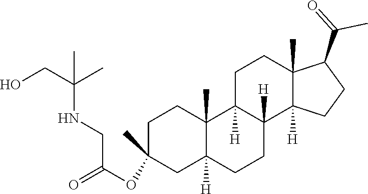 |
| 133 | 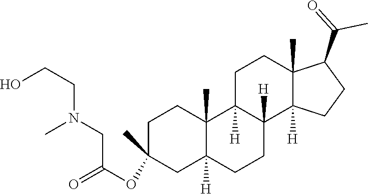 |
| 134 | 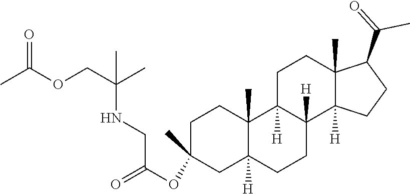 |
| 135 | 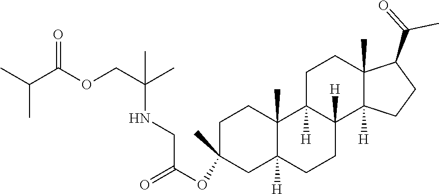 |
| 136 | 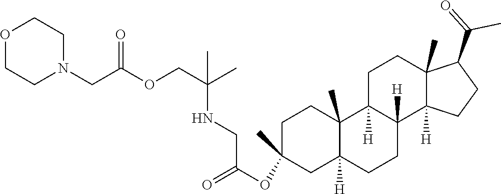 |
| 137 | 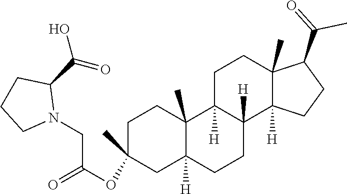 |
| 138 | 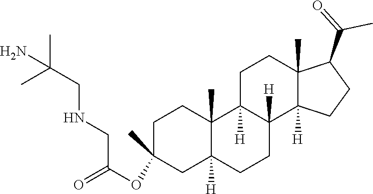 |
| 139 | 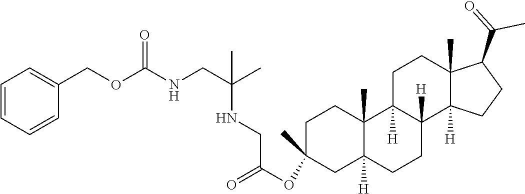 |
| 140 | 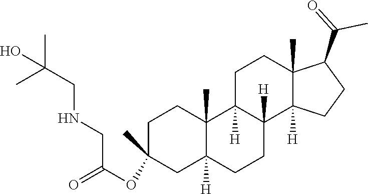 |
| 141 | 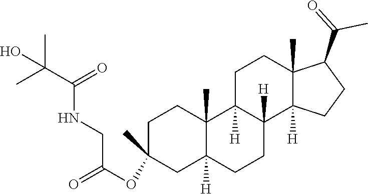 |
| 142 | 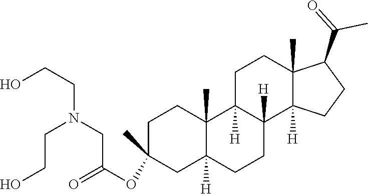 |
| 143 | 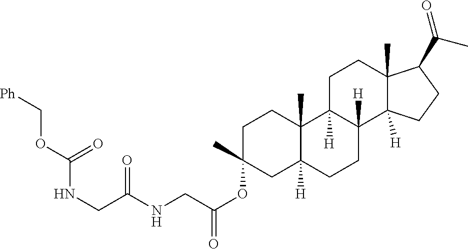 |
| 144 | 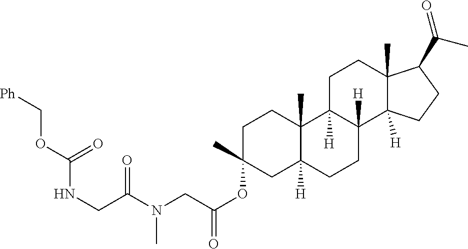 |
| 145 | 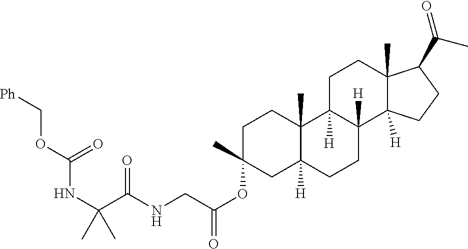 |
| 146 | 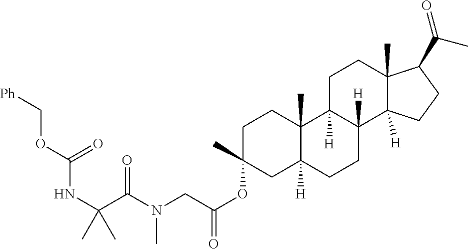 |
| 147 | 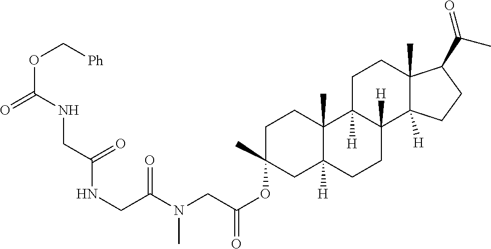 |
| 148 | 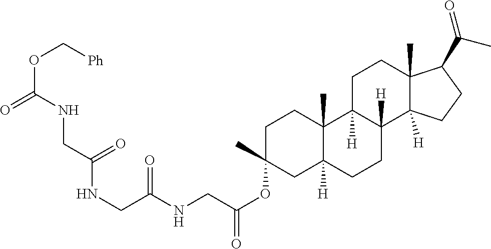 |
| 149 | 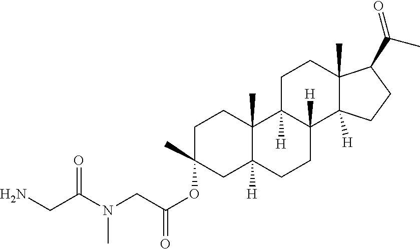 |
| 150 | 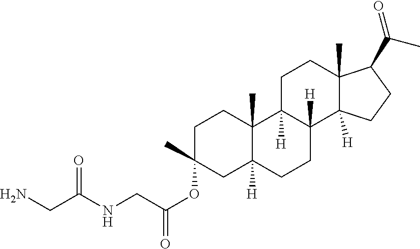 |
| 151 | 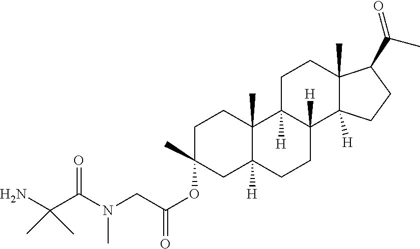 |
| 152 | 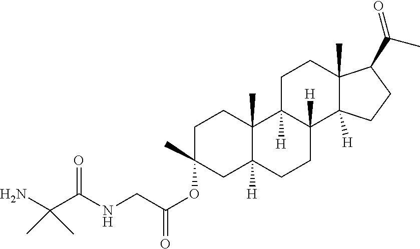 |
| 153 | 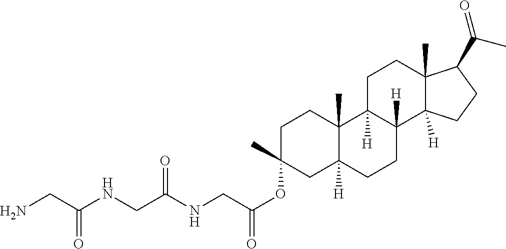 |
| 154 | 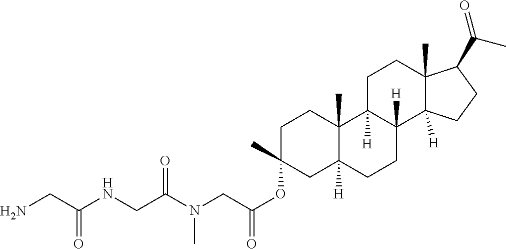 |
| 200 | 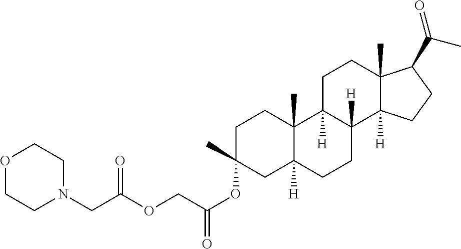 |
| 201 | 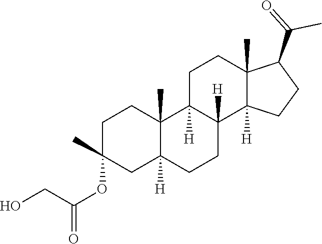 |
| 202 | 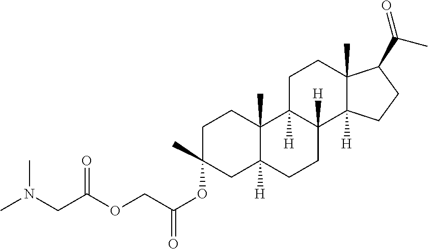 |
| 203 | 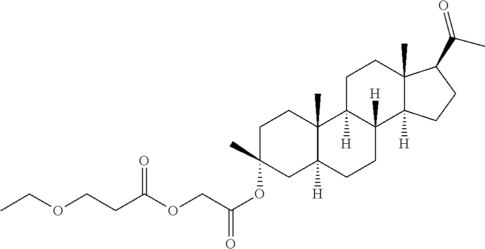 |
| 204 | 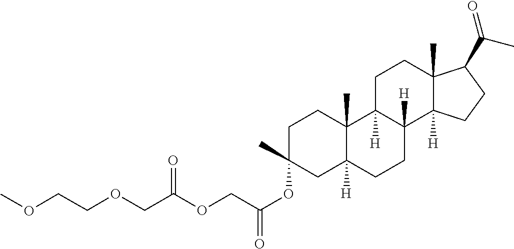 |
| 205 | 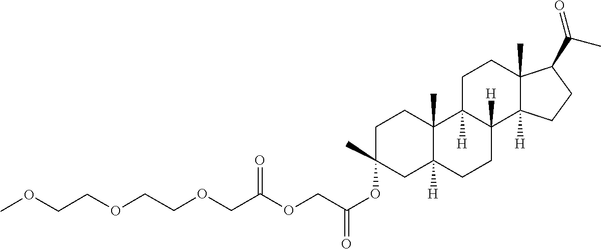 |
| 206 | 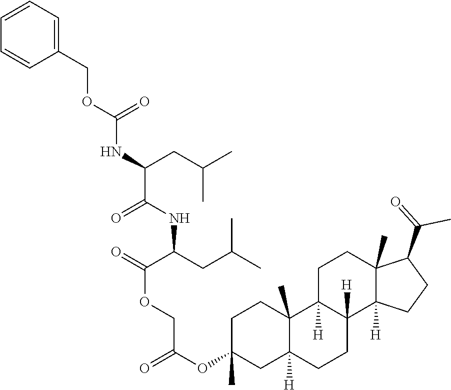 |
| 207 | 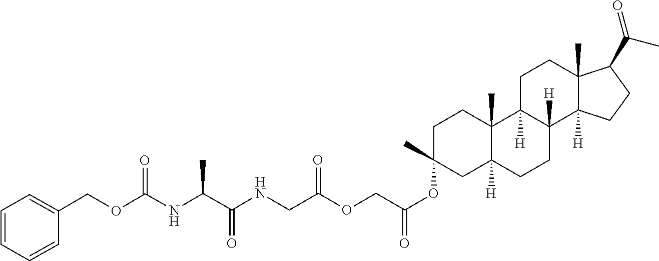 |
| 208 | 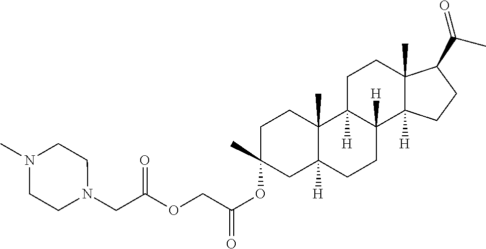 |
| 209 | 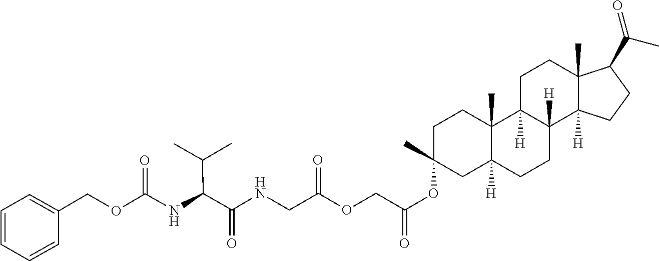 |
| 210 | 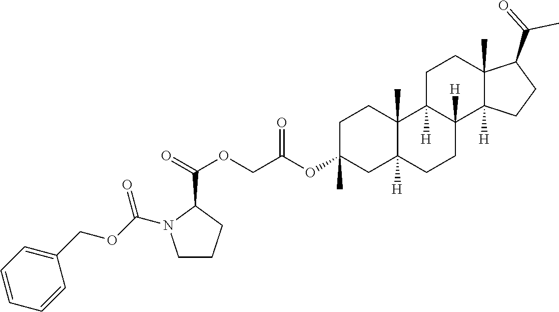 |
| 211 | 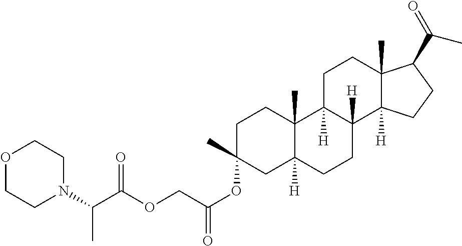 |
| 212 | 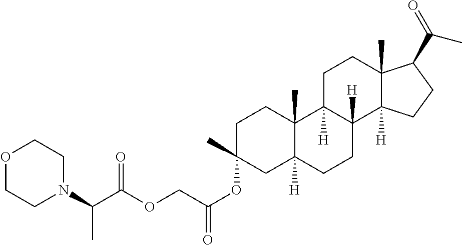 |
| 213 | 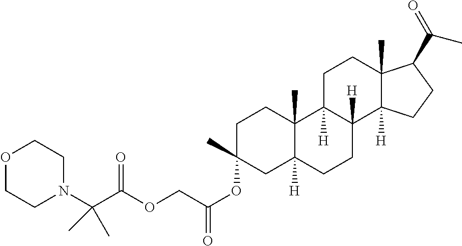 |
| 214 | 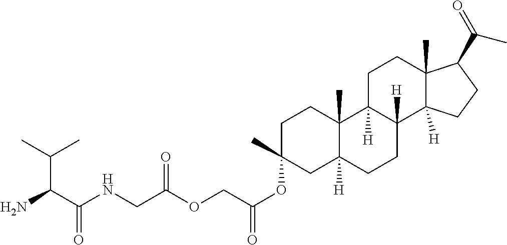 |
| 215 | 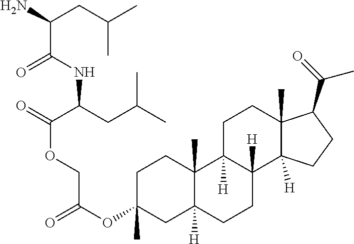 |
| 216 | 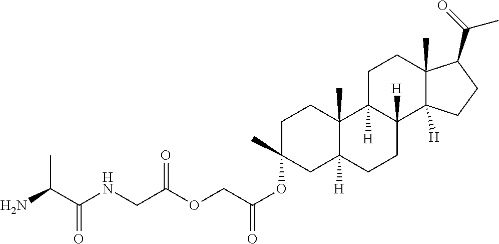 |
| 217 | 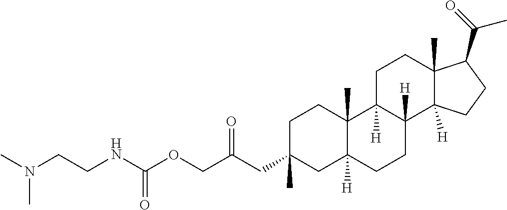 |
| 218 | 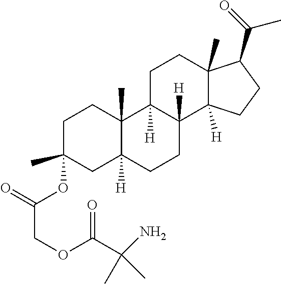 |
| 219 | 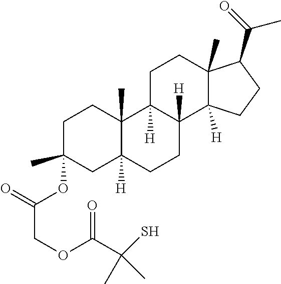 |
| 220 | 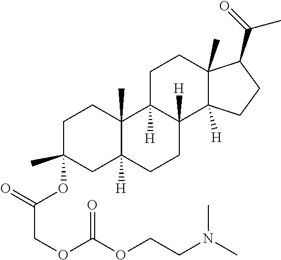 |
| 221 | 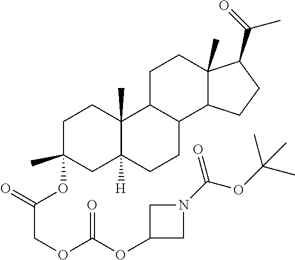 [SMILES: CC(=O)[C@H]1CCC2C3CC[C@H]4C[C@](C)(OC(=O)COC(=O)OC5CN(C(=O)OC(C)(C)C)C5)CC[C@]4(C)C3CC[C@@]21C] |
| 222 | 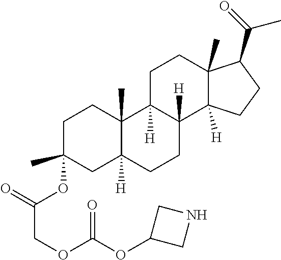 |
| 223 | 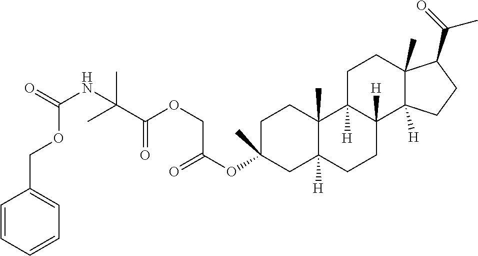 |
| 224 | 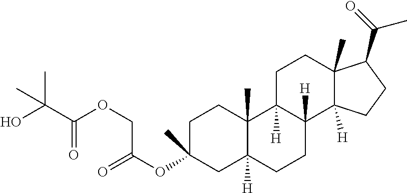 |
| 225 | 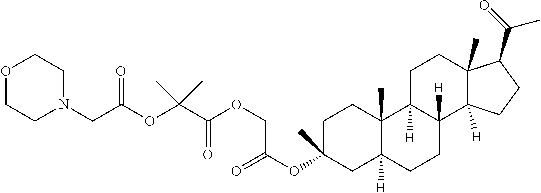 |
| 226 | 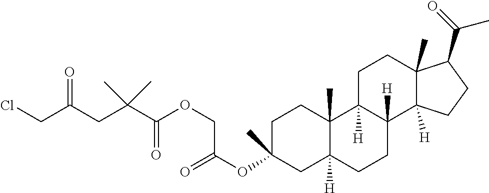 |
| 227 | 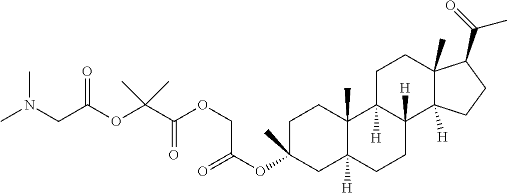 |
| 228 | 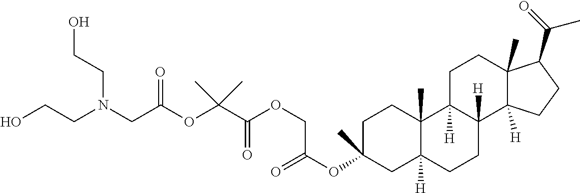 |
| 229 | 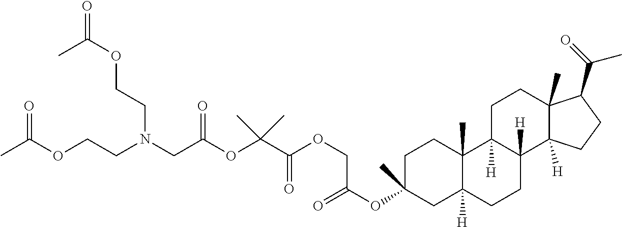 |
| 230 | 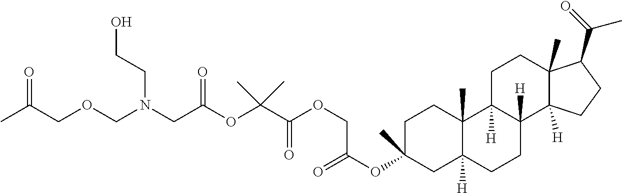 |
| 231 | 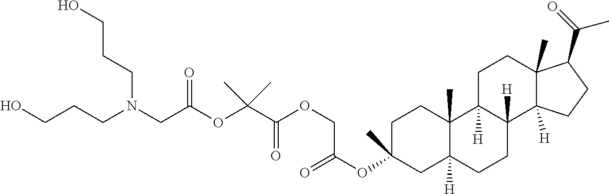 |
| 232 | 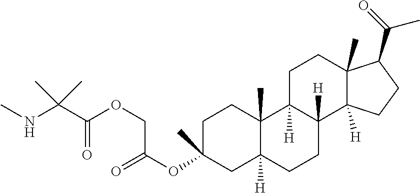 |
| 233 | 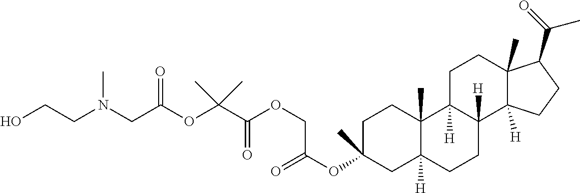 |
| 234 | 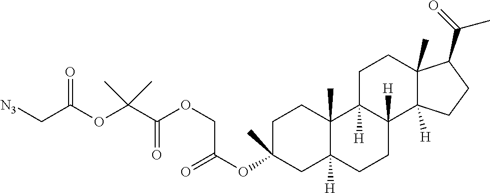 |
| 235 | 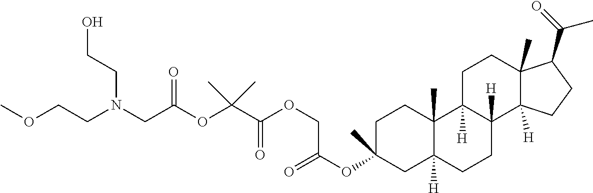 |
| 236 | 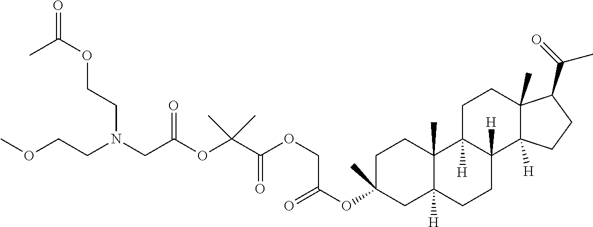 |
| 237 | 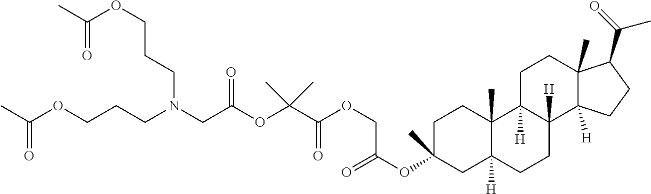 |
| 238 | 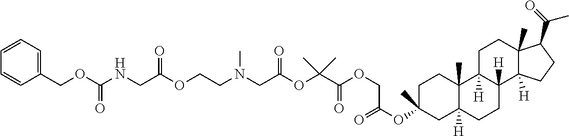 |
| 239 | 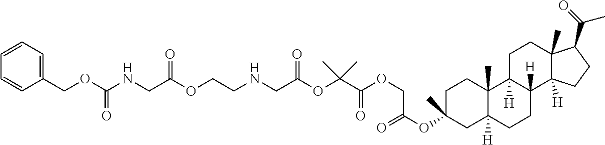 |
| 240 | 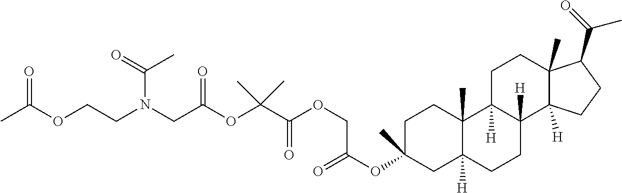 |
| 241 | 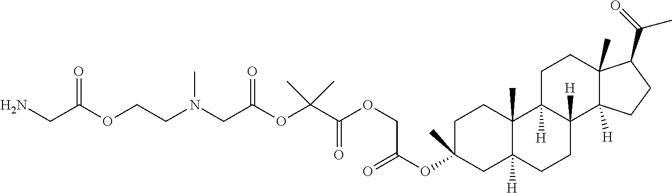 |
| 242 | 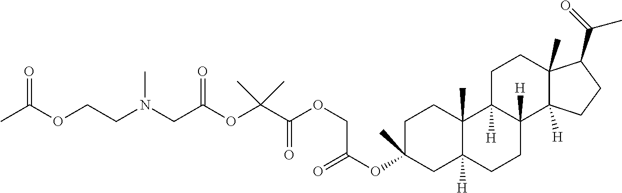 |
| 243 | 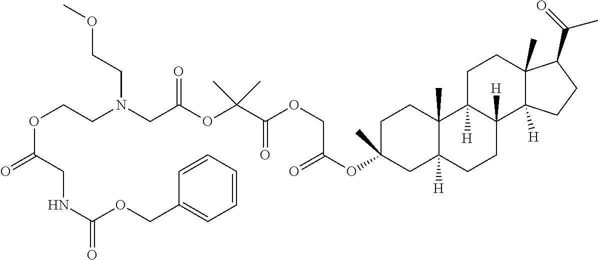 |
| 244 | 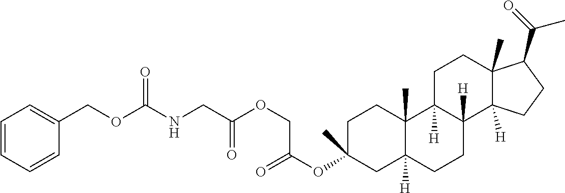 |
| 245 | 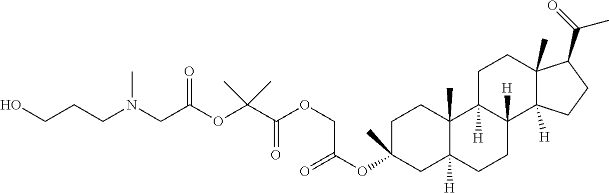 |
| 246 | 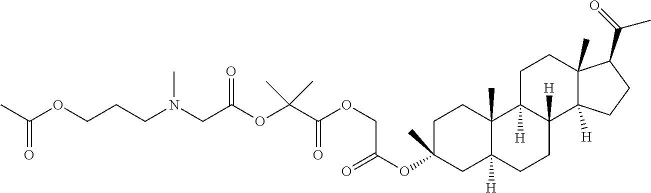 |
| 247 | 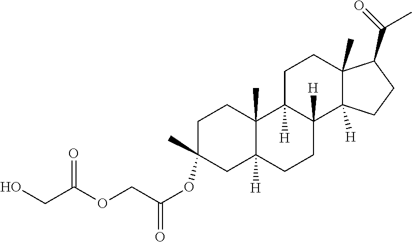 |
| 248 | 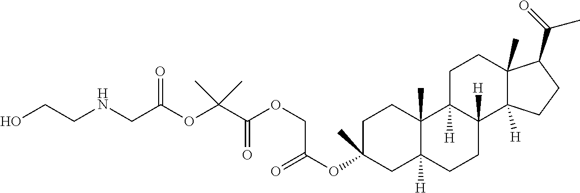 |
| 300 | 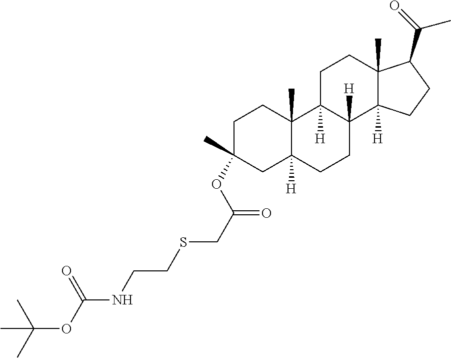 |
| 301 | 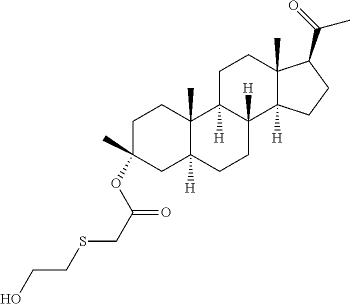 |
| 302 | 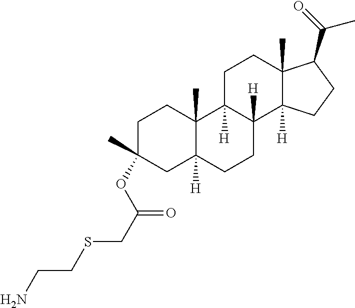 |
| 303 | 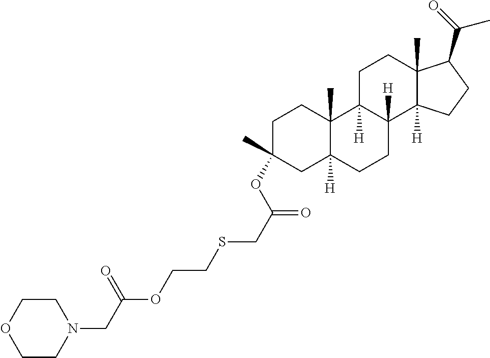 |
| 304 | 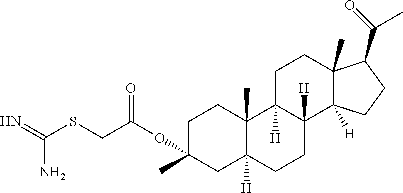 |
| 305 | 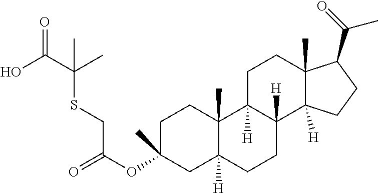 |
| 306 | 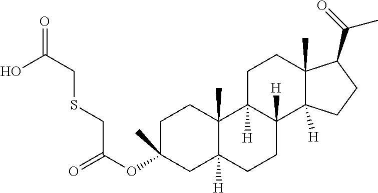 |
| 307 | 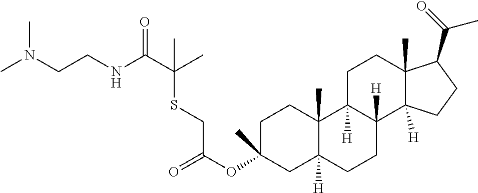 |
| 308 | 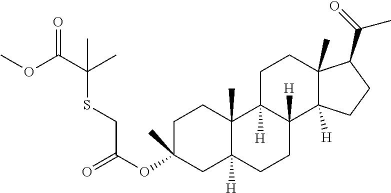 |
| 309 | 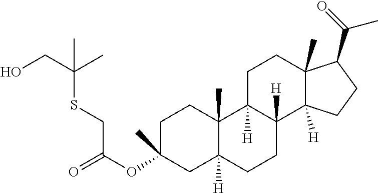 |
| 310 | 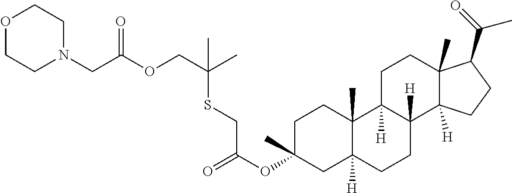 |
| 400 | 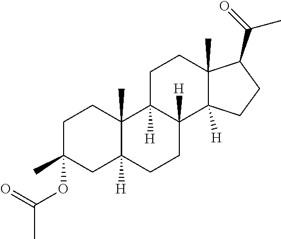 |
| 401 | 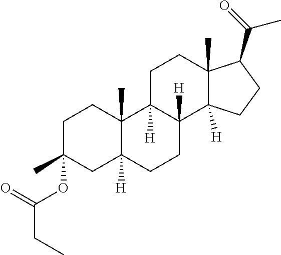 |
| 402 | 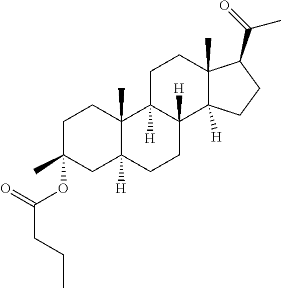 |
| 403 | 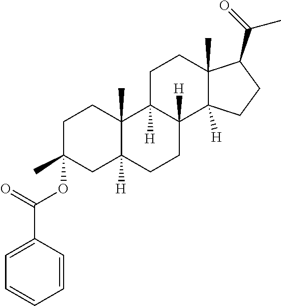 |
| 404 | 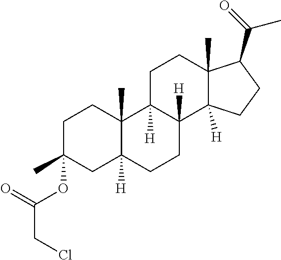 |
| 405 | 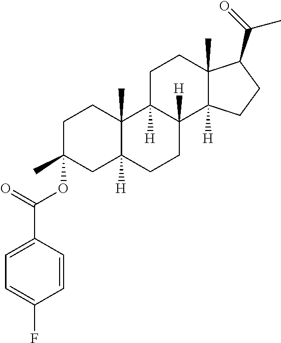 |
| 406 | 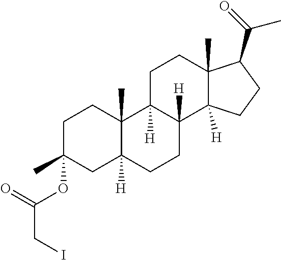 |
| 407 | 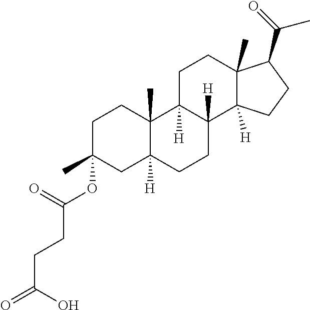 |
| 408 | 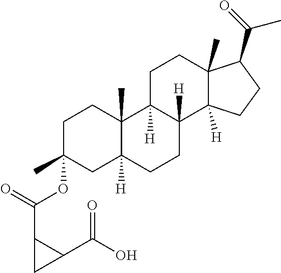 |
| 409 | 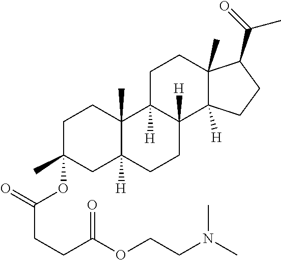 |
| 410 | 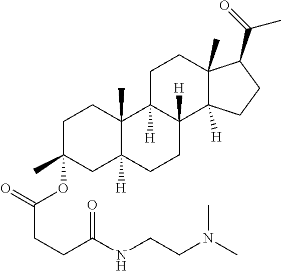 |
| 411 | 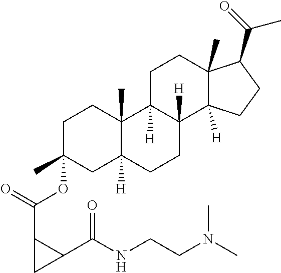 |
| 412 | 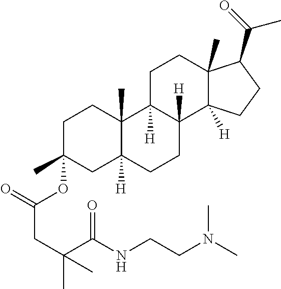 |
| 413 | 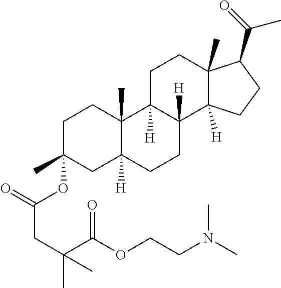 |
| 414 | 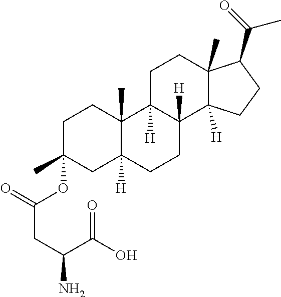 |
| 415 | 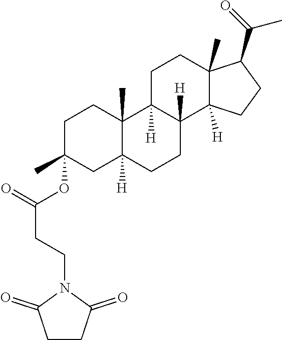 |
| 416 | 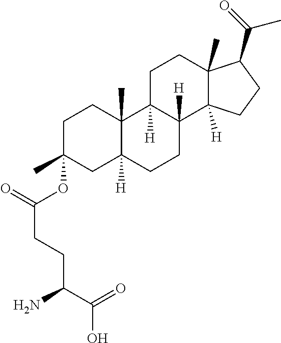 |
| 417 | 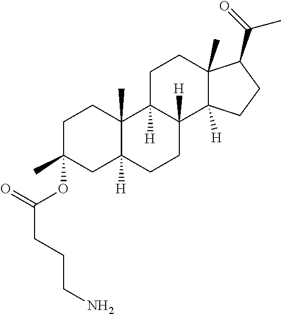 |
| 418 | 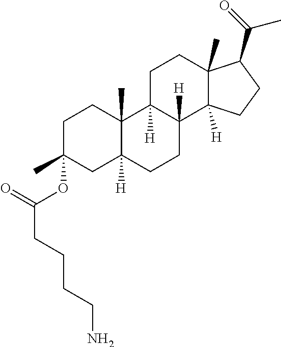 |
| 419 | 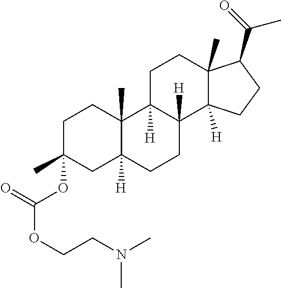 |
| 420 | 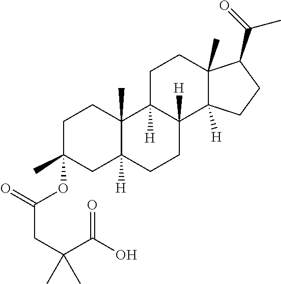 |
| 500 | 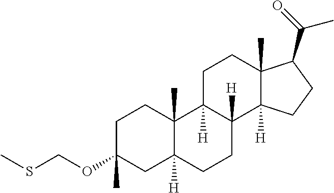 |
| 501 | 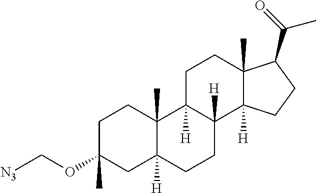 |
| 502 | 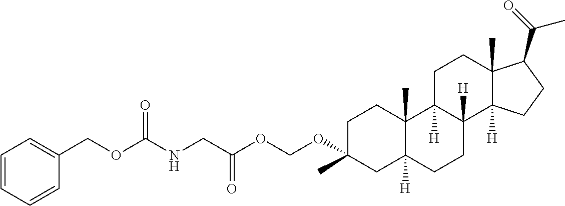 |
| 503 | 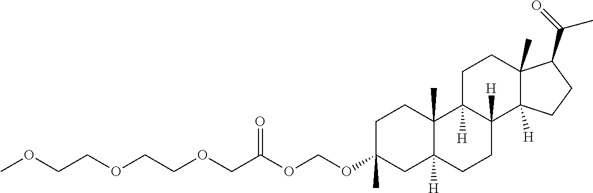 |
| 504 | 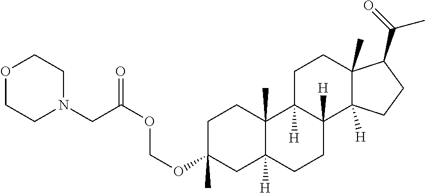 |

In some embodiments, the compound of Formulae (I), (I-I), and (IA) is Compound 100, or a pharmaceutically acceptable salt (e.g., an HCl or TFA salt), solvate, hydrate, tautomer, or stereoisomer thereof. In some embodiments, the compound of Formulas (I), (I-I), and (IA) is Compound 101, or a pharmaceutically acceptable salt (e.g., an HCl or TFA salt), solvate, hydrate, tautomer, or stereoisomer thereof. In some embodiments, the compound of Formulas (I), (I-I), and (IA) is Compound 102, or a pharmaceutically acceptable salt (e.g., an HCl or TFA salt), solvate, hydrate, tautomer, or stereoisomer thereof. In some embodiments, the compound of Formulas (I), (I-I), (IA), (I-II), (I-IIa), and (I-IIc) is Compound 103, or a pharmaceutically acceptable salt (e.g., an HCl or TFA salt), solvate, hydrate, tautomer, or stereoisomer thereof. In some embodiments, the compound of Formulas (I), (I-I), (IA), (I-II), (I-IIa), and (I-IIc) is Compound 104, or a pharmaceutically acceptable salt (e.g., an HCl or TFA salt), solvate, hydrate, tautomer, or stereoisomer thereof. In some embodiments, the compound of Formulas (I), (I-I), (IA), (I-II), (I-IIa), and (I-IIc) is Compound 105, or a pharmaceutically acceptable salt (e.g., an HCl or TFA salt), solvate, hydrate, tautomer, or stereoisomer thereof. In some embodiments, the compound of Formulas (I), (I-I), (IA), (I-II), (I-IIa), and (I-IIc) is Compound 106, or a pharmaceutically acceptable salt (e.g., an HCl or TFA salt), solvate, hydrate, tautomer, or stereoisomer thereof. In some embodiments, the compound of Formulas (I), (I-I), (IA), (I-II), and (I-IIa) is Compound 107, or a pharmaceutically acceptable salt (e.g., an HCl or TFA salt), solvate, hydrate, tautomer, or stereoisomer thereof. In some embodiments, the compound of Formulas (I), (I-I), (IA), (I-II), (I-IIa), and (I-IIc) is Compound 108, or a pharmaceutically acceptable salt (e.g., an HCl or TFA salt), solvate, hydrate, tautomer, or stereoisomer thereof. In some embodiments, the compound of Formulas (I), (I-I), (IA), (I-II), (I-IIa), and (I-IIc) is Compound 109, or a pharmaceutically acceptable salt (e.g., an HCl or TFA salt), solvate, hydrate, tautomer, or stereoisomer thereof. In some embodiments, the compound of Formulas (I), (I-I), (IA), (I-II), (I-IIa), and (I-IIc) is Compound 110, or a pharmaceutically acceptable salt (e.g., an HCl or TFA salt), solvate, hydrate, tautomer, or stereoisomer thereof. In some embodiments, the compound of Formulas (I), (I-I), (IA), and (IA-a) is Compound 111, or a pharmaceutically acceptable salt (e.g., an HCl or TFA salt), solvate, hydrate, tautomer, or stereoisomer thereof. In some embodiments, the compound of Formulas (I), (I-I), (IA), (I-II), and (I-IIa) is Compound 112, or a pharmaceutically acceptable salt (e.g., an HCl or TFA salt), solvate, hydrate, tautomer, or stereoisomer thereof. In some embodiments, the compound of Formulas (I), (I-I), (IA), (I-II), and (I-IIa) is Compound 113, or a pharmaceutically acceptable salt (e.g., an HCl or TFA salt), solvate, hydrate, tautomer, or stereoisomer thereof. In some embodiments, the compound of Formulas (I), (I-I), and (IA) is Compound 114, or a pharmaceutically acceptable salt (e.g., an HCl or TFA salt), solvate, hydrate, tautomer, or stereoisomer thereof. In some embodiments, the compound of Formulas (I), (I-I), (IA), (I-II), and (I-IIa) is Compound 115, or a pharmaceutically acceptable salt (e.g., an HCl or TFA salt), solvate, hydrate, tautomer, or stereoisomer thereof. In some embodiments, the compound of Formulas (I), (I-I), and (IA) is Compound 116, or a pharmaceutically acceptable salt (e.g., an HCl or TFA salt), solvate, hydrate, tautomer, or stereoisomer thereof. In some embodiments, the compound of Formulas (I), (I-I), (IA), (I-II), and (I-IIa) is Compound 117, or a pharmaceutically acceptable salt (e.g., an HCl or TFA salt), solvate, hydrate, tautomer, or stereoisomer thereof. In some embodiments, the compound of Formulas (I), (I-I), (IA), (I-II), and (I-IIa) is Compound 118, or a pharmaceutically acceptable salt (e.g., an HCl or TFA salt), solvate, hydrate, tautomer, or stereoisomer thereof. In some embodiments, the compound of Formulas (I), (I-I), and (IA) is Compound 119, or a pharmaceutically acceptable salt (e.g., an HCl or TFA salt), solvate, hydrate, tautomer, or stereoisomer thereof. In some embodiments, the compound of Formulas (I), (I-I), (IA), (IA-a), (IA-b), (I-II), (I-IIa), and (I-IIc) is Compound 120, or a pharmaceutically acceptable salt (e.g., an HCl or TFA salt), solvate, hydrate, tautomer, or stereoisomer thereof. In some embodiments, the compound of Formulas (I), (I-I), and (IA) is Compound 121, or a pharmaceutically acceptable salt (e.g., an HCl or TFA salt), solvate, hydrate, tautomer, or stereoisomer thereof. In some embodiments, the compound of Formulas (I), (I-I), (IA), (I-II), and (I-IIa) is Compound 122, or a pharmaceutically acceptable salt (e.g., an HCl or TFA salt), solvate, hydrate, tautomer, or stereoisomer thereof. In some embodiments, the compound of Formulas (I), (I-I), and (IA) is Compound 123, or a pharmaceutically acceptable salt (e.g., an HCl or TFA salt), solvate, hydrate, tautomer, or stereoisomer thereof. In some embodiments, the compound of Formulas (I), (I-I), (IA), (I-II), (I-IIa), and (I-IIc) is Compound 124, or a pharmaceutically acceptable salt (e.g., an HCl or TFA salt), solvate, hydrate, tautomer, or stereoisomer thereof. In some embodiments, the compound of Formulas (I), (I-I), (IA), (I-II), (I-IIa), and (I-IIc) is Compound 125, or a pharmaceutically acceptable salt (e.g., an HCl or TFA salt), solvate, hydrate, tautomer, or stereoisomer thereof. In some embodiments, the compound of Formulas (I), (I-I), (IA), (I-II), (I-IIa), and (I-IIc) is Compound 126, or a pharmaceutically acceptable salt (e.g., an HCl or TFA salt), solvate, hydrate, tautomer, or stereoisomer thereof. In some embodiments, the compound of Formulas (I), (I-I), (IA), (I-II), (I-IIa), and (I-IIc) is Compound 127, or a pharmaceutically acceptable salt (e.g., an HCl or TFA salt), solvate, hydrate, tautomer, or stereoisomer thereof. In some embodiments, the compound of Formulas (I), (I-I), (IA), (I-II), (I-IIa), and (I-IIc) is Compound 128, or a pharmaceutically acceptable salt (e.g., an HCl or TFA salt), solvate, hydrate, tautomer, or stereoisomer thereof. In some embodiments, the compound of Formulas (I), (I-I), (IA), (I-II), (I-IIa), and (I-IIc) is Compound 129, or a pharmaceutically acceptable salt (e.g., an HCl or TFA salt), solvate, hydrate, tautomer, or stereoisomer thereof. In some embodiments, the compound of Formulas (I), (I-I), (IA), (IA-a), (IA-b), (I-II), (I-IIa), and (I-IIc) is Compound 130, or a pharmaceutically acceptable salt (e.g., an HCl or TFA salt), solvate, hydrate, tautomer, or stereoisomer thereof. In some embodiments, the compound of Formulas (I), (I-I), (IA), (IA-a), (IA-b), (I-II), (I-IIa), and (I-IIc) is Compound 131, or a pharmaceutically acceptable salt (e.g., an HCl or TFA salt), solvate, hydrate, tautomer, or stereoisomer thereof. In some embodiments, the compound of Formulas (I), (I-I), (IA), (IA-a), (I-II), and (I-IIa) is Compound 132, or a pharmaceutically acceptable salt (e.g., an HCl or TFA salt), solvate, hydrate, tautomer, or stereoisomer thereof. In some embodiments, the compound of Formulas (I), (I-I), (IA), (IA-a), (I-II), and (I-IIa) is Compound 133, or a pharmaceutically acceptable salt (e.g., an HCl or TFA salt), solvate, hydrate, tautomer, or stereoisomer thereof. In some embodiments, the compound of Formulas (I), (I-I), (IA), (IA-a), (I-II), and (I-IIa) is Compound 134, or a pharmaceutically acceptable salt (e.g., an HCl or TFA salt), solvate, hydrate, tautomer, or stereoisomer thereof. In some embodiments, the compound of Formulas (I), (I-I), (IA), (IA-a), (I-II), and (I-IIa) is Compound 135, or a pharmaceutically acceptable salt (e.g., an HCl or TFA salt), solvate, hydrate, tautomer, or stereoisomer thereof. In some embodiments, the compound of Formulas (I), (I-I), (IA), (IA-a), (I-hI), and (I-IIa) is Compound 136, or a pharmaceutically acceptable salt (e.g., an HCl or TFA salt), solvate, hydrate, tautomer, or stereoisomer thereof. In some embodiments, the compound of Formulas (I), (I-I), (IA), (I-II), (I-IIa), and (I-IIc) is Compound 137, or a pharmaceutically acceptable salt (e.g., an HCl or TFA salt), solvate, hydrate, tautomer, or stereoisomer thereof. In some embodiments, the compound of Formulas (I), (I-I), (IA), (IA-a), (I-II), and (I-IIa) is Compound 138, or a pharmaceutically acceptable salt (e.g., an HCl or TFA salt), solvate, hydrate, tautomer, or stereoisomer thereof. In some embodiments, the compound of Formulas (I), (I-I), (IA), (IA-a), (I-II), and (I-IIa) is Compound 139, or a pharmaceutically acceptable salt (e.g., an HCl or TFA salt), solvate, hydrate, tautomer, or stereoisomer thereof. In some embodiments, the compound of Formulas (I), (I-I), (IA), (IA-a), (I-II), and (I-IIa) is Compound 140, or a pharmaceutically acceptable salt (e.g., an HCl or TFA salt), solvate, hydrate, tautomer, or stereoisomer thereof. In some embodiments, the compound of Formulas (I), (I-I), (IA), (IA-a), (I-II), and (I-IIa) is Compound 141, or a pharmaceutically acceptable salt (e.g., an HCl or TFA salt), solvate, hydrate, tautomer, or stereoisomer thereof. In some embodiments, the compound of Formulas (I), (I-I), (IA), (I-II), and (I-IIa) is Compound 142, or a pharmaceutically acceptable salt (e.g., an HCl or TFA salt), solvate, hydrate, tautomer, or stereoisomer thereof. In some embodiments, the compound of Formulas (I), (I-I), (IA), (I-II), and (I-IIa) is Compound 143, or a pharmaceutically acceptable salt (e.g., an HCl or TFA salt), solvate, hydrate, tautomer, or stereoisomer thereof. In some embodiments, the compound of Formulas (I), (I-I), (IA), (I-II), and (I-IIa) is Compound 144, or a pharmaceutically acceptable salt (e.g., an HCl or TFA salt), solvate, hydrate, tautomer, or stereoisomer thereof. In some embodiments, the compound of Formulas (I), (I-I), (IA), (I-II), and (I-IIa) is Compound 145, or a pharmaceutically acceptable salt (e.g., an HCl or TFA salt), solvate, hydrate, tautomer, or stereoisomer thereof. In some embodiments, the compound of Formulas (I), (I-I), (IA), (I-II), and (I-IIa) is Compound 146, or a pharmaceutically acceptable salt (e.g., an HCl or TFA salt), solvate, hydrate, tautomer, or stereoisomer thereof. In some embodiments, the compound of Formulas (I), (I-I), (IA), (I-II), and (I-IIa) is Compound 147, or a pharmaceutically acceptable salt (e.g., an HCl or TFA salt), solvate, hydrate, tautomer, or stereoisomer thereof. In some embodiments, the compound of Formulas (I), (I-I), (IA), (I-II), and (I-IIa) is Compound 148, or a pharmaceutically acceptable salt (e.g., an HCl or TFA salt), solvate, hydrate, tautomer, or stereoisomer thereof. In some embodiments, the compound of Formulas (I), (I-I), (IA), (I-II), and (I-IIa) is Compound 149, or a pharmaceutically acceptable salt (e.g., an HCl or TFA salt), solvate, hydrate, tautomer, or stereoisomer thereof. In some embodiments, the compound of Formulas (I), (I-I), (IA), (I-II), and (I-IIa) is Compound 150, or a pharmaceutically acceptable salt (e.g., an HCl or TFA salt), solvate, hydrate, tautomer, or stereoisomer thereof. In some embodiments, the compound of Formulas (I), (I-I), (IA), (I-II), and (I-IIa) is Compound 151, or a pharmaceutically acceptable salt (e.g., an HCl or TFA salt), solvate, hydrate, tautomer, or stereoisomer thereof. In some embodiments, the compound of Formulas (I), (I-I), (IA), (I-II), and (I-IIa) is Compound 152, or a pharmaceutically acceptable salt (e.g., an HCl or TFA salt), solvate, hydrate, tautomer, or stereoisomer thereof. In some embodiments, the compound of Formulas (I), (I-I), (IA), (I-II), and (I-IIa) is Compound 153, or a pharmaceutically acceptable salt (e.g., an HCl or TFA salt), solvate, hydrate, tautomer, or stereoisomer thereof. In some embodiments, the compound of Formulas (I), (I-I), (IA), (I-II), and (I-IIa) is Compound 154, or a pharmaceutically acceptable salt (e.g., an HCl or TFA salt), solvate, hydrate, tautomer, or stereoisomer thereof.

In some embodiments, the compound of Formulas (I), (I-I), (IB), (I-II), and (I-IIb) is Compound 200, or a pharmaceutically acceptable salt (e.g., an HCl or TFA salt), solvate, hydrate, tautomer, or stereoisomer thereof. In some embodiments, the compound of Formulas (I), (I-I), and (IB) is Compound 201, or a pharmaceutically acceptable salt (e.g., an HCl or TFA salt), solvate, hydrate, tautomer, or stereoisomer thereof. In some embodiments, the compound of Formulas (I), (I-I), (IB), (I-II), and (I-IIb) is Compound 202, or a pharmaceutically acceptable salt (e.g., an HCl or TFA salt), solvate, hydrate, tautomer, or stereoisomer thereof. In some embodiments, the compound of Formulas (I), (I-I), and (IB) is Compound 203, or a pharmaceutically acceptable salt (e.g., an HCl or TFA salt), solvate, hydrate, tautomer, or stereoisomer thereof. In some embodiments, the compound of Formulas (I), (I-I), (IB), (I-II), and (I-IIb) is Compound 204, or a pharmaceutically acceptable salt (e.g., an HCl or TFA salt), solvate, hydrate, tautomer, or stereoisomer thereof. In some embodiments, the compound of Formulas (I), (I-I), (IB), (I-II), and (I-IIb) is Compound 205, or a pharmaceutically acceptable salt (e.g., an HCl or TFA salt), solvate, hydrate, tautomer, or stereoisomer thereof. In some embodiments, the compound of Formulas (I), (I-I), (IB), (I-II), and (I-IIb) is Compound 206, or a pharmaceutically acceptable salt (e.g., an HCl or TFA salt), solvate, hydrate, tautomer, or stereoisomer thereof. In some embodiments, the compound of Formulas (I), (I-I), (IB), (I-II), and (I-IIb) is Compound 207, or a pharmaceutically acceptable salt (e.g., an HCl or TFA salt), solvate, hydrate, tautomer, or stereoisomer thereof. In some embodiments, the compound of Formulas (I), (I-I), (IB), (I-II), and (I-IIb) is Compound 208, or a pharmaceutically acceptable salt (e.g., an HCl or TFA salt), solvate, hydrate, tautomer, or stereoisomer thereof. In some embodiments, the compound of Formulas (I), (I-I), (IB), (I-II), and (I-IIb) is Compound 209, or a pharmaceutically acceptable salt (e.g., an HCl or TFA salt), solvate, hydrate, tautomer, or stereoisomer thereof. In some embodiments, the compound of Formulas (I), (I-I), and (IB) is Compound 210, or a pharmaceutically acceptable salt (e.g., an HCl or TFA salt), solvate, hydrate, tautomer, or stereoisomer thereof. In some embodiments, the compound of Formulas (I), (I-I), (IB), (I-II), and (I-IIb) is Compound 211, or a pharmaceutically acceptable salt (e.g., an HCl or TFA salt), solvate, hydrate, tautomer, or stereoisomer thereof. In some embodiments, the compound of Formulas (I), (I-I), (IB), (I-II), and (I-IIb) is Compound 212, or a pharmaceutically acceptable salt (e.g., an HCl or TFA salt), solvate, hydrate, tautomer, or stereoisomer thereof. In some embodiments, the compound of Formulas (I), (I-I), (IB), (I-II), (I-IIb), and (I-IIe) is Compound 213, or a pharmaceutically acceptable salt (e.g., an HCl or TFA salt), solvate, hydrate, tautomer, or stereoisomer thereof. In some embodiments, the compound of Formulas (I), (I-I), (IB), (I-II), and (I-IIb) is Compound 214, or a pharmaceutically acceptable salt (e.g., an HCl or TFA salt), solvate, hydrate, tautomer, or stereoisomer thereof. In some embodiments, the compound of Formulas (I), (I-I), (IB), (I-II), and (I-IIb) is Compound 215, or a pharmaceutically acceptable salt (e.g., an HCl or TFA salt), solvate, hydrate, tautomer, or stereoisomer thereof. In some embodiments, the compound of Formulas (I), (I-I), (IB), (I-II), and (I-IIb) is Compound 216, or a pharmaceutically acceptable salt (e.g., an HCl or TFA salt), solvate, hydrate, tautomer, or stereoisomer thereof. In some embodiments, the compound of Formulas (I), (I-I), and (IB) is Compound 217, or a pharmaceutically acceptable salt (e.g., an HCl or TFA salt), solvate, hydrate, tautomer, or stereoisomer thereof. In some embodiments, the compound of Formulas (I), (I-I), (IB), (I-II), (I-IIb), and (I-IIe) is Compound 218, or a pharmaceutically acceptable salt (e.g., an HCl or TFA salt), solvate, hydrate, tautomer, or stereoisomer thereof. In some embodiments, the compound of Formulas (I), (I-I), (IB), (I-II), (I-IIb), and (I-IIe) is Compound 219, or a pharmaceutically acceptable salt (e.g., an HCl or TFA salt), solvate, hydrate, tautomer, or stereoisomer thereof. In some embodiments, the compound of Formulas (I), (I-I), and (IB) is Compound 220, or a pharmaceutically acceptable salt (e.g., an HCl or TFA salt), solvate, hydrate, tautomer, or stereoisomer thereof. In some embodiments, the compound of Formulas (I), (I-I), and (IB) is Compound 221, or a pharmaceutically acceptable salt (e.g., an HCl or TFA salt), solvate, hydrate, tautomer, or stereoisomer thereof. In some embodiments, the compound of Formulas (I), (I-I), and (IB) is Compound 222, or a pharmaceutically acceptable salt (e.g., an HCl or TFA salt), solvate, hydrate, tautomer, or stereoisomer thereof. In some embodiments, the compound of Formulas (I), (I-I), (IB), (I-II), (I-IIb), and (I-IIe) is Compound 223, or a pharmaceutically acceptable salt (e.g., an HCl or TFA salt), solvate, hydrate, tautomer, or stereoisomer thereof. In some embodiments, the compound of Formulas (I), (I-I), (IB), (I-II), (I-IIb), and (I-IIe) is Compound 224, or a pharmaceutically acceptable salt (e.g., an HCl or TFA salt), solvate, hydrate, tautomer, or stereoisomer thereof. In some embodiments, the compound of Formulas (I), (I-I), (IB), (I-II), (I-IIb), and (I-IIe) is Compound 225, or a pharmaceutically acceptable salt (e.g., an HCl or TFA salt), solvate, hydrate, tautomer, or stereoisomer thereof. In some embodiments, the compound of Formulas (I), (I-I), (IB), (I-II), (I-IIb), and (I-IIe) is Compound 226, or a pharmaceutically acceptable salt (e.g., an HCl or TFA salt), solvate, hydrate, tautomer, or stereoisomer thereof. In some embodiments, the compound of Formulas (I), (I-I), (IB), (I-II), (I-IIb), and (I-IIe) is Compound 227, or a pharmaceutically acceptable salt (e.g., an HCl or TFA salt), solvate, hydrate, tautomer, or stereoisomer thereof. In some embodiments, the compound of Formulas (I), (I-I), (IB), (I-II), (I-IIb), and (I-IIe) is Compound 228, or a pharmaceutically acceptable salt (e.g., an HCl or TFA salt), solvate, hydrate, tautomer, or stereoisomer thereof. In some embodiments, the compound of Formulas (I), (I-I), (IB), (I-II), (I-IIb), and (I-IIe) is Compound 229, or a pharmaceutically acceptable salt (e.g., an HCl or TFA salt), solvate, hydrate, tautomer, or stereoisomer thereof. In some embodiments, the compound of Formulas (I), (I-I), (IB), (I-II), (I-IIb), and (I-IIe) is Compound 230, or a pharmaceutically acceptable salt (e.g., an HCl or TFA salt), solvate, hydrate, tautomer, or stereoisomer thereof. In some embodiments, the compound of Formulas (I), (I-I), (IB), (I-II), (I-IIb), and (I-IIe) is Compound 231, or a pharmaceutically acceptable salt (e.g., an HCl or TFA salt), solvate, hydrate, tautomer, or stereoisomer thereof. In some embodiments, the compound of Formulas (I), (I-I), (IB), (I-II), (I-IIb), and (I-IIe) is Compound 232, or a pharmaceutically acceptable salt (e.g., an HCl or TFA salt), solvate, hydrate, tautomer, or stereoisomer thereof. In some embodiments, the compound of Formulas (I), (I-I), (IB), (I-II), (I-IIb), and (I-IIe) is Compound 233, or a pharmaceutically acceptable salt (e.g., an HCl or TFA salt), solvate, hydrate, tautomer, or stereoisomer thereof. In some embodiments, the compound of Formulas (I), (I-I), (IB), (I-II), (I-IIb), and (I-IIe) is Compound 234, or a pharmaceutically acceptable salt (e.g., an HCl or TFA salt), solvate, hydrate, tautomer, or stereoisomer thereof. In some embodiments, the compound of Formulas (I), (I-I), (IB), (I-II), (I-IIb), and (I-IIe) is Compound 235, or a pharmaceutically acceptable salt (e.g., an HCl or TFA salt), solvate, hydrate, tautomer, or stereoisomer thereof. In some embodiments, the compound of Formulas (I), (I-I), (IB), (I-II), (I-IIb), and (I-IIe) is Compound 236, or a pharmaceutically acceptable salt (e.g., an HCl or TFA salt), solvate, hydrate, tautomer, or stereoisomer thereof. In some embodiments, the compound of Formulas (I), (I-I), (IB), (I-II), (I-IIb), and (I-IIe) is Compound 237, or a pharmaceutically acceptable salt (e.g., an HCl or TFA salt), solvate, hydrate, tautomer, or stereoisomer thereof. In some embodiments, the compound of Formulas (I), (I-I), (IB), (I-II), (I-IIb), and (I-IIe) is Compound 238, or a pharmaceutically acceptable salt (e.g., an HCl or TFA salt), solvate, hydrate, tautomer, or stereoisomer thereof. In some embodiments, the compound of Formulas (I), (I-I), (IB), (I-II), (I-IIb), and (I-IIe) is Compound 239, or a pharmaceutically acceptable salt (e.g., an HCl or TFA salt), solvate, hydrate, tautomer, or stereoisomer thereof. In some embodiments, the compound of Formulas (I), (I-I), (IB), (I-II), (I-IIb), and (I-IIe) is Compound 240, or a pharmaceutically acceptable salt (e.g., an HCl or TFA salt), solvate, hydrate, tautomer, or stereoisomer thereof. In some embodiments, the compound of Formulas (I), (I-I), (IB), (I-II), (I-IIb), and (I-IIe) is Compound 241, or a pharmaceutically acceptable salt (e.g., an HCl or TFA salt), solvate, hydrate, tautomer, or stereoisomer thereof. In some embodiments, the compound of Formulas (I), (I-I), (IB), (I-II), (I-IIb), and (I-IIe) is Compound 242, or a pharmaceutically acceptable salt (e.g., an HCl or TFA salt), solvate, hydrate, tautomer, or stereoisomer thereof. In some embodiments, the compound of Formulas (I), (I-I), (IB), (I-II), (I-IIb), and (I-IIe) is Compound 243, or a pharmaceutically acceptable salt (e.g., an HCl or TFA salt), solvate, hydrate, tautomer, or stereoisomer thereof. In some embodiments, the compound of Formulas (I), (I-I), (IB), (I-II), and (I-IIb) is Compound 244, or a pharmaceutically acceptable salt (e.g., an HCl or TFA salt), solvate, hydrate, tautomer, or stereoisomer thereof. In some embodiments, the compound of Formulas (I), (I-I), (IB), (I-II), (I-IIb), (I-IIe) is Compound 245, or a pharmaceutically acceptable salt (e.g., an HCl or TFA salt), solvate, hydrate, tautomer, or stereoisomer thereof. In some embodiments, the compound of Formulas (I), (I-I), (IB), (I-II), (I-IIb), and (I-IIe) is Compound 246, or a pharmaceutically acceptable salt (e.g., an HCl or TFA salt), solvate, hydrate, tautomer, or stereoisomer thereof. In some embodiments, the compound of Formulas (I), (I-I), (IB), (I-II), and (I-IIb) is Compound 247, or a pharmaceutically acceptable salt (e.g., an HCl or TFA salt), solvate, hydrate, tautomer, or stereoisomer thereof.

In some embodiments, the compound of Formulas (I), (I-I), (IC), and (I-II) is Compound 300, or a pharmaceutically acceptable salt (e.g., an HCl or TFA salt), solvate, hydrate, tautomer, or stereoisomer thereof. In some embodiments, the compound of Formulas (I), (I-I), (IC), and (I-II) is Compound 301, or a pharmaceutically acceptable salt (e.g., an HCl or TFA salt), solvate, hydrate, tautomer, or stereoisomer thereof. In some embodiments, the compound of Formulas (I), (I-I), (IC), and (I-II) is Compound 302, or a pharmaceutically acceptable salt (e.g., an HCl or TFA salt), solvate, hydrate, tautomer, or stereoisomer thereof. In some embodiments, the compound of Formulas (I), (I-I), (IC), and (I-II) is Compound 303, or a pharmaceutically acceptable salt (e.g., an HCl or TFA salt), solvate, hydrate, tautomer, or stereoisomer thereof. In some embodiments, the compound of Formulas (I), (I-I), and (IC) is Compound 304, or a pharmaceutically acceptable salt (e.g., an HCl or TFA salt), solvate, hydrate, tautomer, or stereoisomer thereof. In some embodiments, the compound of Formulas (I), (I-I), (IC), and (I-II) is Compound 305, or a pharmaceutically acceptable salt (e.g., an HCl or TFA salt), solvate, hydrate, tautomer, or stereoisomer thereof. In some embodiments, the compound of Formulas (I), (I-I), (IC), and (I-II) is Compound 306, or a pharmaceutically acceptable salt (e.g., an HCl or TFA salt), solvate, hydrate, tautomer, or stereoisomer thereof. In some embodiments, the compound of Formulas (I), (I-I), (IC), and (I-II) is Compound 307, or a pharmaceutically acceptable salt (e.g., an HCl or TFA salt), solvate, hydrate, tautomer, or stereoisomer thereof. In some embodiments, the compound of Formulas (I), (I-I), (IC), and (I-II) is Compound 308, or a pharmaceutically acceptable salt (e.g., an HCl or TFA salt), solvate, hydrate, tautomer, or stereoisomer thereof. In some embodiments, the compound of Formulas (I), (I-I), (IC), and (I-II) is Compound 309, or a pharmaceutically acceptable salt (e.g., an HCl or TFA salt), solvate, hydrate, tautomer, or stereoisomer thereof. In some embodiments, the compound of Formulas (I), (I-I), (IC), and (I-II) is Compound 310, or a pharmaceutically acceptable salt (e.g., an HCl or TFA salt), solvate, hydrate, tautomer, or stereoisomer thereof.

In some embodiments, the compound of Formulas (I), (I-I), and (ID) is Compound 400, or a pharmaceutically acceptable salt (e.g., an HCl or TFA salt), solvate, hydrate, tautomer, or stereoisomer thereof. In some embodiments, the compound of Formulas (I), (I-I), and (ID) is Compound 401, or a pharmaceutically acceptable salt (e.g., an HCl or TFA salt), solvate, hydrate, tautomer, or stereoisomer thereof. In some embodiments, the compound of Formulas (I), (I-I), and (ID) is Compound 402, or a pharmaceutically acceptable salt (e.g., an HCl or TFA salt), solvate, hydrate, tautomer, or stereoisomer thereof. In some embodiments, the compound of Formulas (I), (I-I), and (ID) is Compound 403, or a pharmaceutically acceptable salt (e.g., an HCl or TFA salt), solvate, hydrate, tautomer, or stereoisomer thereof. In some embodiments, the compound of Formulas (I), (I-I), and (ID) is Compound 404, or a pharmaceutically acceptable salt (e.g., an HCl or TFA salt), solvate, hydrate, tautomer, or stereoisomer thereof. In some embodiments, the compound of Formulas (I), (I-I), and (ID) is Compound 405, or a pharmaceutically acceptable salt (e.g., an HCl or TFA salt), solvate, hydrate, tautomer, or stereoisomer thereof. In some embodiments, the compound of Formulas (I), (I-I), and (ID) is Compound 406, or a pharmaceutically acceptable salt (e.g., an HCl or TFA salt), solvate, hydrate, tautomer, or stereoisomer thereof. In some embodiments, the compound of Formulas (I), (I-I), and (ID) is Compound 407, or a pharmaceutically acceptable salt (e.g., an HCl or TFA salt), solvate, hydrate, tautomer, or stereoisomer thereof. In some embodiments, the compound of Formulas (I), (I-I), and (ID) is Compound 408, or a pharmaceutically acceptable salt (e.g., an HCl or TFA salt), solvate, hydrate, tautomer, or stereoisomer thereof. In some embodiments, the compound of Formulas (I), (I-I), and (ID) is Compound 409, or a pharmaceutically acceptable salt (e.g., an HCl or TFA salt), solvate, hydrate, tautomer, or stereoisomer thereof. In some embodiments, the compound of Formulas (I), (I-I), and (ID) is Compound 410, or a pharmaceutically acceptable salt (e.g., an HCl or TFA salt), solvate, hydrate, tautomer, or stereoisomer thereof. In some embodiments, the compound of Formulas (I), (I-I), and (ID) is Compound 411, or a pharmaceutically acceptable salt (e.g., an HCl or TFA salt), solvate, hydrate, tautomer, or stereoisomer thereof. In some embodiments, the compound of Formulas (I), (I-I), and (ID) is Compound 412, or a pharmaceutically acceptable salt (e.g., an HCl or TFA salt), solvate, hydrate, tautomer, or stereoisomer thereof. In some embodiments, the compound of Formulas (I), (I-I), and (ID) is Compound 413, or a pharmaceutically acceptable salt (e.g., an HCl or TFA salt), solvate, hydrate, tautomer, or stereoisomer thereof. In some embodiments, the compound of Formulas (I), (I-I), and (ID) is Compound 414, or a pharmaceutically acceptable salt (e.g., an HCl or TFA salt), solvate, hydrate, tautomer, or stereoisomer thereof. In some embodiments, the compound of Formulas (I), (I-I), and (ID) is Compound 415, or a pharmaceutically acceptable salt (e.g., an HCl or TFA salt), solvate, hydrate, tautomer, or stereoisomer thereof. In some embodiments, the compound of Formulas (I), (I-I), (ID), and (I-II) is Compound 416, or a pharmaceutically acceptable salt (e.g., an HCl or TFA salt), solvate, hydrate, tautomer, or stereoisomer thereof. In some embodiments, the compound of Formulas (I), (I-I), and (ID) is Compound 417, or a pharmaceutically acceptable salt (e.g., an HCl or TFA salt), solvate, hydrate, tautomer, or stereoisomer thereof. In some embodiments, the compound of Formulas (I), (I-I), (ID), and (I-II) is Compound 418, or a pharmaceutically acceptable salt (e.g., an HCl or TFA salt), solvate, hydrate, tautomer, or stereoisomer thereof. In some embodiments, the compound of Formulas (I), (I-I), and (ID) is Compound 419, or a pharmaceutically acceptable salt (e.g., an HCl or TFA salt), solvate, hydrate, tautomer, or stereoisomer thereof. In some embodiments, the compound of Formulas (I), (I-I), and (ID) is Compound 420, or a pharmaceutically acceptable salt (e.g., an HCl or TFA salt), solvate, hydrate, tautomer, or stereoisomer thereof.

In some embodiments, the compound of Formulas (I), (I-I), and (IE) is Compound 500, or a pharmaceutically acceptable salt (e.g., an HCl or TFA salt), solvate, hydrate, tautomer, or stereoisomer thereof. In some embodiments, the compound of Formulas (I), (I-I), and (IE) is Compound 501, or a pharmaceutically acceptable salt (e.g., an HCl or TFA salt), solvate, hydrate, tautomer, or stereoisomer thereof. In some embodiments, the compound of Formulas (I), (I-I), and (IE) is Compound 502, or a pharmaceutically acceptable salt (e.g., an HCl or TFA salt), solvate, hydrate, tautomer, or stereoisomer thereof. In some embodiments, the compound of Formulas (I), (I-I), and (IE) is Compound 503, or a pharmaceutically acceptable salt (e.g., an HCl or TFA salt), solvate, hydrate, tautomer, or stereoisomer thereof. In some embodiments, the compound of Formulas (I), (I-I), and (IE) is Compound 504, or a pharmaceutically acceptable salt (e.g., an HCl or TFA salt), solvate, hydrate, tautomer, or stereoisomer thereof.

### Pharmaceutical Compositions and Kits

The present disclosure provides pharmaceutical compositions comprising a compound disclosed herein (e.g., a Compound of Formulae (I), (I-I), (IA), (IA-a), (IA-b), (IB), (IC), (ID), (IE), (I-II), (I-IIa), (I-IIb), (I-IIc), (I-IId), (I-IIe), or a compound listed in Table 1, or a pharmaceutically acceptable salt, solvate, hydrate, tautomer, or stereoisomer thereof), and optionally a pharmaceutically acceptable excipient.

In some aspects, a pharmaceutical composition described herein comprises a Compound of Formula (I), or a pharmaceutically acceptable salt, solvate, hydrate, tautomer, or stereoisomer thereof, and optionally a pharmaceutically acceptable excipient.

In some aspects, a pharmaceutical composition described herein comprises a Compound of Formula (I-I), or a pharmaceutically acceptable salt, solvate, hydrate, tautomer, or stereoisomer thereof, and optionally a pharmaceutically acceptable excipient.

In some aspects, a pharmaceutical composition described herein comprises a Compound of Formula (IA), or a pharmaceutically acceptable salt, solvate, hydrate, tautomer, or stereoisomer thereof, and optionally a pharmaceutically acceptable excipient. In some aspects, a pharmaceutical composition described herein comprises a Compound of Formula (IA-a), or a pharmaceutically acceptable salt, solvate, hydrate, tautomer, or stereoisomer thereof, and optionally a pharmaceutically acceptable excipient. In some aspects, a pharmaceutical composition described herein comprises a Compound of Formula (IA-b), or a pharmaceutically acceptable salt, solvate, hydrate, tautomer, or stereoisomer thereof, and optionally a pharmaceutically acceptable excipient.

In some aspects, a pharmaceutical composition described herein comprises a Compound of Formula (IB), or a pharmaceutically acceptable salt, solvate, hydrate, tautomer, or stereoisomer thereof, and optionally a pharmaceutically acceptable excipient.

In some aspects, a pharmaceutical composition described herein comprises a Compound of Formula (IC), or a pharmaceutically acceptable salt, solvate, hydrate, tautomer, or stereoisomer thereof, and optionally a pharmaceutically acceptable excipient.

In some aspects, a pharmaceutical composition described herein comprises a Compound of Formula (ID), or a pharmaceutically acceptable salt, solvate, hydrate, tautomer, or stereoisomer thereof, and optionally a pharmaceutically acceptable excipient.

In some aspects, a pharmaceutical composition described herein comprises a Compound of Formula (IE), or a pharmaceutically acceptable salt, solvate, hydrate, tautomer, or stereoisomer thereof, and optionally a pharmaceutically acceptable excipient.

In some aspects, a pharmaceutical composition described herein comprises a Compound of Formula (I-II), or a pharmaceutically acceptable salt, solvate, hydrate, tautomer, or stereoisomer thereof, and optionally a pharmaceutically acceptable excipient. In some aspects, a pharmaceutical composition described herein comprises a Compound of Formula (I-IIa), or a pharmaceutically acceptable salt, solvate, hydrate, tautomer, or stereoisomer thereof, and optionally a pharmaceutically acceptable excipient. In some aspects, a pharmaceutical composition described herein comprises a Compound of Formula (I-IIb), or a pharmaceutically acceptable salt, solvate, hydrate, tautomer, or stereoisomer thereof, and optionally a pharmaceutically acceptable excipient. In some aspects, a pharmaceutical composition described herein comprises a Compound of Formula (I-IIc), or a pharmaceutically acceptable salt, solvate, hydrate, tautomer, or stereoisomer thereof, and optionally a pharmaceutically acceptable excipient. In some aspects, a pharmaceutical composition described herein comprises a Compound of Formula (I-IId), or a pharmaceutically acceptable salt, solvate, hydrate, tautomer, or stereoisomer thereof, and optionally a pharmaceutically acceptable excipient. In some aspects, a pharmaceutical composition described herein comprises a Compound of Formula (I-IIe), or a pharmaceutically acceptable salt, solvate, hydrate, tautomer, or stereoisomer thereof, and optionally a pharmaceutically acceptable excipient.

In some aspects, a pharmaceutical composition described herein comprises a compound that is listed in Table 1, or a pharmaceutically acceptable salt, solvate, hydrate, tautomer, or stereoisomer thereof, and optionally a pharmaceutically acceptable excipient. The compound, or pharmaceutically acceptable salt, solvate, hydrate, tautomer, or stereoisomer thereof, may be provided in an effective amount in the pharmaceutical composition.

Pharmaceutical compositions of the present disclosure may be prepared by any suitable method known in the art. Such methods can involve a step of bringing the compound (the “active ingredient,” e.g., a compound of Formulae (I), (I-I), (IA), (IA-a), (IA-b), (IB), (IC), (ID), (IE), (I-II), (I-IIa), (I-IIb), (I-IIc), (I-IId), (I-IIe), or Table 1), into association with one or more pharmaceutically acceptable excipients (e.g., a carrier or binding agent), and may further involve a step of shaping and/or packaging the composition into a single- or multi-dose unit.

Pharmaceutical compositions may be prepared, packaged, and/or sold in bulk as a single unit dose or a plurality of single unit doses. A single unit dose describes a discrete amount of the pharmaceutical composition comprising a predetermined amount of the active ingredient (e.g., a compound disclosed herein, e.g., a compound of Formulae (I), (I-I), (IA), (IA-a), (IA-b), (IB), (IC), (ID), (IE), (I-II), (I-IIa), (I-IIb), (I-IIc), (I-IId), (I-IIe), or Table 1). The amount of the active ingredient is typically equal to the dosage of the active ingredient would be administered to a subject, or a convenient fraction of such a dosage.

Relative amounts of the active ingredient, and the one or more pharmaceutically acceptable excipients, in a pharmaceutical composition of the present disclosure will vary depending on the identity, size, and/or condition of the subject to be treated, and also depending on the route by which the composition is to be administered. For example, the pharmaceutical composition may comprise between about 0.1 wt % and about 100 wt % of the active ingredient (e.g., a compound of Formulae (I), (I-I), (IA), (IA-a), (IA-b), (IB), (IC), (ID), (IE), (I-II), (I-IIa), (I-IIb), (I-IIc), (I-IId), (I-IIe), or Table 1).

Compositions of the present disclosure may be administered orally, parenterally (including, without limitation, subcutaneously, intramuscularly, intravenously, and intradermally), by inhalation (e.g., oral inhalation), topically, rectally, nasally, buccally, vaginally, or by an implanted reservoir. In some embodiments, the compositions of the present disclosure are administered orally. In some embodiments, the compositions of the present disclosure are administered intravenously.

Pharmaceutical compositions of the present disclosure may be orally administered in any acceptable dosage form including, without limitation, capsules, tablets, caplets, powders, lozenges, pastilles, suspensions (e.g., aqueous suspensions), or solutions. Oral dosage forms can include a pharmaceutically acceptable excipient (e.g., a pharmaceutically acceptable excipient disclosed herein), e.g., carriers, lubricating agents, diluents, emulsifiers, suspending agents, flavoring agents, coloring agents, disintegrants, or the like. For example, an oral dose form disclosed herein may contain one or more of lactose, corn starch, magnesium stearate, a sweetener, a dye, a polyether (e.g., polyethylene glycol, e.g., PEG-300), a cyclodextrin (e.g., CAPTISOL®), or a polysorbate (e.g., a TWEEN®, e.g., TWEEN-20®).

Pharmaceutical compositions of the present disclosure may be intravenously administered in any acceptable dosage form including, without limitation, aqueous solutions. Intravenous dosage forms can include a pharmaceutically acceptable excipient (e.g., a pharmaceutically acceptable excipient disclosed herein), e.g., carriers, diluents, or the like. For example, an intravenous dose form disclosed herein may contain one or more of a polyether (e.g., polyethylene glycol, e.g., PEG-300), a cyclodextrin (e.g., CAPTISOL®), or a polysorbate (e.g., a TWEEN®, e.g., TWEEN-20®).

A pharmaceutical composition or dosage form disclosed herein (e.g., intravenous dosage form) may be a composition or dosage form that does not contain a solubility-enhancing agent, such as a cyclodextrin (e.g., CAPTISOL®). For example, a pharmaceutical composition or dosage form disclosed herein (e.g., intravenous dosage form) may include a compound disclosed herein without cyclodextrin (e.g., CAPTISOL®), or with a relatively low amount of the cyclodextrin, such as a compound:cyclodextrin ratio of less than 1:1, e.g., less than 2:1, less than 3:1, less than 4:1, less than 5:1, less than 6:1, less than 7:1, less than 8:1, less than 9:1, less than 10:1, less than 15:1, less than 20:1, less than 25:1, less than 50:1, less than 99:1, or lower.

A pharmaceutical composition or dosage form (e.g., intravenous dosage form) disclosed herein may comprise relatively low amounts of a solubility-enhancing agent (e.g., a cyclodextrin, such as CAPTISOL®), or no solubility-enhancing agent. For example, a pharmaceutical composition or dosage form (e.g., intravenous dosage form) disclosed herein may comprise no cyclodextrin (e.g., CAPTISOL®), or relatively low amounts of cyclodextrin (e.g., CAPTISOL®) such as less than 25 wt %, less than 20 wt %, less than 15 wt %, less than 10 wt %, less than 5 wt %, less than 4 wt %, less than 3 wt %, less than 2 wt %, less than 1 wt %, less than 0.5 wt %, of cyclodextrin in the composition.

Although the descriptions of pharmaceutical compositions provided herein are primarily directed to pharmaceutical compositions which are suitable for administration to humans, it will be understood that such compositions are generally suitable for administration to other animals. It will also be well understood that modifications of pharmaceutical compositions are possible to render the compositions suitable for administration to various animals, and the ordinarily skilled person can design and/or perform such modification with ordinary experimentation.

Compounds disclosed herein are typically formulated in a dosage unit form, e.g., a single unit dosage form, for ease of administration and uniformity of dosage. However, it will be understood that the total daily usage of the compounds or compositions of the present disclosure will be decided by the attending physician within the scope of sound medical judgment. The specific therapeutically effective dose level for any particular subject or organism will depend upon a variety of factors including, without limitation: the disease or disorder being treated, and the severity of the disease or disorder being treated; the activity of the specific compound or composition being administered; the specific compound or composition being administered; the age, body weight, general heath, sex, and/or diet of the subject; the time of administration, route of administration, and rate of excretion or metabolism of the specific compound or composition being administered; the duration of treatment, drugs used in combination or coincidental with the specific compound or composition being administered; and like factors known in the art.

The amount of a compound or composition required to achieve an effective amount will vary from subject to subject, depending on various factors including, e.g., species, age, and general condition of the subject; severity of the side-effects or disorder; identity of the particular compound or composition; mode of administration, and the like. The desired dosage may be delivered one or more times a day, e.g., once daily, twice daily, thrice daily, etc., or may be delivered less frequently, e.g., every other day, every third day, every week, every two weeks, every three weeks, monthly, etc. The desired dosage may be delivered on an as needed basis. The desired dosage may also be delivered using multiple administrations, e.g., two, three, four, five, or more administrations.

An effective amount of a compound for administration will vary depending on the body weight of the subject. For example, an effective amount of a compound for administration one or more times a day to an adult human weighing 70 kg may comprise about 0.0001 mg to about 3 g, e.g., about 0.0001 mg to about 2 g, about 0.0001 mg to about 1 g, about 0.001 mg to about 1 g, about 0.01 mg to about 1 g, about 0.1 mg to about 1 g, about 1 mg to about 1 g, about 1 mg to about 750 mg, about 1 mg to about 500 mg, about 1 mg to about 400 mg, about 1 mg to about 300 mg, about 1 mg to about 250 mg, about 1 mg to about 200 mg, about 1 mg to about 150 mg, about 1 mg to about 100 mg, about 5 mg to about 100 mg, or about 10 mg to about 100 mg, of a compound per unit dosage form.

It will be understood that dose ranges described herein provide guidance for the administration to an adult, and that the amount to be administered to, e.g., pediatric subjects, can be determined by a medical practitioner or person skilled in the art, and may be lower or the same as that administered to an adult.

The compounds and compositions described herein may be administered in combination with one or more additional pharmaceutical agents. The compounds and compositions disclosed herein can be administered in combination with additional pharmaceutical agents that improve their bioavailability, improve effectiveness of treatment, reduce or modify metabolism of the compound, or inhibit excretion and/or modify distribution of the compound within the body. It will be understood that the additional pharmaceutical agent employed may achieve a desired effect for the same disorder as the compound or composition, and/or may achieve different effects.

The compound or composition can be administered concurrently with, prior to, or subsequent to, one or more additional pharmaceutical agents, which can be useful as, e.g., combination therapies. Pharmaceutical agents include therapeutically active agents. Pharmaceutical agents also include prophylactically active agents. Each additional pharmaceutical agent may be administered at a dose and/or at a time determined for that pharmaceutical agent. The additional pharmaceutical agents may also be administered together with each other and/or with the compound or composition described herein in a single dose or administered separately in different doses. The particular combination to employ in a regimen will take into account compatibility of the inventive compound with the additional pharmaceutical agents and/or the desired therapeutic and/or prophylactic effect to be achieved. In general, it is expected that the additional pharmaceutical agents utilized in combination be utilized at levels that do not exceed the levels and which they are utilized individually. In some aspects, the levels utilized in combination will be lower than those utilized individually.

Also encompassed by the present disclosure are kits (e.g., pharmaceutical packs). The kits may be useful for preventing and/or treating a disease or disorder disclosed herein, such as a neurological disorder (e.g., epilepsy). The kits may comprise a compound or composition disclosed herein and a container (e.g., a blister pack, vial, bottle, ampule, dispenser package, syringe, or other suitable container). In some embodiments, kits may further include a second container comprising a pharmaceutical excipient, e.g., for dilution or suspension of the compound or composition. In some embodiments, the compound or composition provided in the container, and the second container, are combined to form one unit dosage form, or a multi-unit dosage form.

### Pharmacokinetics

The compounds and pharmaceutical compositions disclosed herein can provide enhanced pharmacokinetic and pharmacodynamic profiles, e.g., relative to ganaxolone. In particular, compounds and pharmaceutical compositions disclosed herein have superior oral bioavailability compared to ganaxolone. Following administration (e.g., oral administration or intravenous administration), the compounds disclosed herein may be converted in vivo (e.g., by metabolic processes) to ganaxolone. The compounds disclosed herein may be referred to as ganaxolone prodrugs. Thus, administering a compound disclosed herein may provide a higher amount of serum ganaxolone (e.g., as determined by ganaxolone Cₘₐₓ or AUC), compared to administering ganaxolone. Without wishing to be bound by theory, it is believed that the increased bioavailability of the compounds disclosed herein ultimately increase exposure of ganaxolone and/or improve maintenance of the ganaxolone steady state following administration (e.g., by oral administration), relative to the amounts achieved by administration of ganaxolone itself. This effect can be due in part to the increased solubility, hydrophilicity, and/or rate of absorption of the compounds disclosed herein, compared to ganaxolone, and their ability to be converted to ganaxolone by metabolic processes.

Similarly, the compounds and pharmaceutical compositions disclosed herein can provide a higher concentration of ganaxolone in the CNS, following administration (e.g., by intravenous or oral administration) to a subject, relative to the concentration achieved when ganaxolone is administered to the subject in an equivalent amount and by the same route of administration.

The compounds and pharmaceutical compositions disclosed herein can have a bioavailability, e.g., oral bioavailability, with respect to serum concentration of ganaxolone, that is higher as compared to the bioavailability of administering ganaxolone itself (e.g., as determined by ganaxolone Cₘₐₓ or AUC), by at least about 5%, about 10%, about 15%, about 20%, about 30%, about 40%, about 50%, about 60%, about 70, about 80%, about 90%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 325%, about 350%, about 375%, about 400%, about 425%, about 450%, about 475%, about 500%, about 525%, about 550%, about 575%, about 600%, about 625%, about 650%, about 675%, about 700%, about 725%, about 750%, about 775%, about 800%, about 825%, about 850%, about 875%, about 900%, about 925%, about 950%, about 975%, about 1000% or greater.

Because the compounds disclosed herein may be converted in situ to ganaxolone (e.g., by metabolic processes), the enhanced bioavailability achieved by the compounds disclosed herein can be measured by the Cₘₐₓ levels of ganaxolone in the blood plasma and/or area under the curve (AUC) level of ganaxolone, in addition to measuring the Cₘₐₓ and AUC levels of the prodrug compound itself. In some embodiments, the compounds and pharmaceutical compositions disclosed herein may increase the Cₘₐₓ and/or the AUC of ganaxolone, relative to the Cₘₐₓ and/or AUC achieved following oral administration of a composition comprising ganaxolone.

The compounds and pharmaceutical compositions described herein may result in a maximum ganaxolone plasma concentration (Cₘₐₓ) from about 100 ng/mL to about 1200 ng/mL. For example, the compound may achieve a Cₘₐₓ of about 100 ng/mL, about 110 ng/mL, about 120 ng/mL, about 130 ng/mL, about 140 ng/mL, about 150 ng/mL, about 160 ng/mL, about 170 ng/mL, about 180 ng/mL, about 190 ng/mL, about 200 ng/mL, about 210 ng/mL, about 220 ng/mL, about 230 ng/mL, about 240 ng/mL, about 250 ng/mL, about 260 ng/mL, about 270 ng/mL, about 280 ng/mL, about 290 ng/mL, about 300 ng/mL, about 310 ng/mL, about 320 ng/mL, about 330 ng/mL, about 340 ng/mL, about 350 ng/mL, about 360 ng/mL, about 370 ng/mL, about 380 ng/mL, about 390 ng/mL, about 400 ng/mL, about 410 ng/mL, about 420 ng/mL, about 430 ng/mL, about 440 ng/mL, about 450 ng/mL, about 460 ng/mL, about 470 ng/mL, about 480 ng/mL, about 490 ng/mL, about 500 ng/mL, about 510 ng/mL, about 520 ng/mL, about 530 ng/mL, about 540 ng/mL, about 550 ng/mL, about 560 ng/mL, 570 ng/mL, about 580 ng/mL, about 590 ng/mL, about 600 ng/mL, about 610 ng/mL, about 620 ng/mL, about 630 ng/mL, about 640 ng/mL, about 650 ng/mL, about 660 ng/mL, about 670 ng/mL, about 680 ng/mL, about 690 ng/mL, about 700 ng/mL, about 710 ng/mL, about 720 ng/mL, about 730 ng/mL, about 740 ng/mL, about 750 ng/mL, about 760 ng/mL, about 770 ng/mL, about 780 ng/mL, about 790 ng/mL, 800 ng/mL, about 810 ng/mL, about 820 ng/mL, about 830 ng/mL, about 840 ng/mL, about 850 ng/mL, about 860 ng/mL, about 870 ng/mL, about 880 ng/mL, about 890 ng/mL, about 900 ng/mL, about 910 ng/mL, about 920 ng/mL, about 930 ng/mL, 940 ng/mL, about 950 ng/mL, about 960 ng/mL, about 970 ng/mL, about 980 ng/mL, about 990 ng/mL, about 1000 ng/mL, about 1100 ng/mL, or about 1200 ng/mL, or greater.

The compounds and pharmaceutical compositions described herein can increase ganaxolone Cₘₐₓ by at least about 1 fold, about 2 fold, about 3 fold, about 4 fold, about 5 fold, about 6 fold, about 7 fold, about 8 fold, about 9 fold, about 10 fold, about 11 fold, about 12 fold, about 13 fold, about 15 fold, about 16 fold, about 17 fold, about 18 fold, about 19 fold, about 20 fold, or greater, relative to other oral compositions comprising ganaxolone.

The compounds and pharmaceutical compositions disclosed herein can increase the AUC of ganaxolone, relative to AUC achieved following oral administration of another composition comprising ganaxolone. For example, oral administration of a compound or pharmaceutical composition disclosed herein can result in an AUC of ganaxolone of about 100 ng/mL/hr to about 1,200 ng/mL/hr, e.g., about 200 ng/mL/hr to about 1,200 ng/mL/hr, about 300 ng/mL/hr to about 1,200 ng/mL/hr, about 400 ng/mL/hr to about 1,200 ng/mL/hr, about 500 ng/mL/hr to about 1,200 ng/mL/hr, about 600 ng/mL/hr to about 1,200 ng/mL/hr, about 700 ng/mL/hr to about 1,200 ng/mL/hr, about 800 ng/mL/hr to about 1,200 ng/mL/hr, about 900 ng/mL/hr to about 1,200 ng/mL/hr. The AUC may refers to the AUC calculated to the last measured concentration (AUC_{0,t}), such as over a period of 24 hours (AUC₀₋₂₄).

The compounds and pharmaceutical compositions disclosed herein can be administered at a dose that results in an average ganaxolone plasma concentration (Cₐᵥₑ) of about 100 ng/mL to about 1,200 ng/mL, about 200 ng/mL to about 1,200 ng/mL, about 300 ng/mL to about 1,200 ng/mL, about 400 ng/mL to about 1,200 ng/mL, about 500 ng/mL to about 1,200 ng/mL, about 600 ng/mL to about 1,200 ng/mL, about 700 ng/mL to about 1,200 ng/mL, about 800 ng/mL to about 1,200 ng/mL, or about 900 ng/mL to about 1,200 ng/mL.

The compounds and pharmaceutical compositions disclosed herein can be administered at a dose that results in plasma ganaxolone concentration of about 100 ng/mL to about 1,200 ng/mL, about 200 ng/mL to about 1,200 ng/mL, about 300 ng/mL to about 1,200 ng/mL, about 400 ng/mL to about 1,200 ng/mL, about 500 ng/mL to about 1,200 ng/mL, about 600 ng/mL to about 1,200 ng/mL, about 700 ng/mL to about 1,200 ng/mL, about 800 ng/mL to about 1,200 ng/mL, or about 900 ng/mL to about 1,200 ng/mL.

### Methods of Use

The present disclosure also relates to methods of treating a disease or disorder. The methods disclosed herein can comprise administering (e.g., orally or intravenously administering) a therapeutically effective amount of a compound or a pharmaceutical composition disclosed herein, to a subject in need thereof.

The methods can be suitable for treating any type of seizure disorder, epilepsy disorder, genetic epilepsy disorder, epilepsy related disorder, central nervous system disorder, neurological disorder, neurodegenerative disorder, or the like.

Types of disorders, including epilepsy disorders and/or epilepsy-related disorders, the methods disclosed herein can be suitable for treating include, but are not limited to, focal seizure, essential tremor, a generalized seizure, acute repetitive seizures, pediatric epilepsy, reflex epilepsy, benign rolandic epilepsy, status epilepticus, refractory status epilepticus, super-refractory status epilepticus, SCN8 A epilepsy, catamenial epilepsy, Angelman syndrome, benign epilepsy with centro-temporal spikes (BECTS), CDKL5 deficiency disorder (CDKL5 disorder), autosomal dominant nocturnal frontal lobe epilepsy (ADFE), absence epilepsy, childhood absence epilepsy (CAE), Doose syndrome, Dravet syndrome, early myoclonic epilepsy (EME), epilepsy with generalized tonic-clonic seizures, epilepsy with myoclonic-absences, infantile spasms (West syndrome), Landau-Kleffner syndrome, Lennox-Gastaut syndrome (LGS), epilepsy with myoclonic absences, frontal lobe epilepsy, juvenile myoclonic epilepsy (JME), Lyfora progressive myoclonus epilepsy, Ohtahara syndrome, Rett syndrome, intractable childhood epilepsy (ICE), Panayiotopoulos syndrome, Rasmussen's syndrome, progressive myoclonic epilepsies, Ring chromosome 20 syndrome, temporal lobe epilepsy, epilepsy of infancy with migrating focal seizures, epilepsy in Fragile X syndrome, Sturge-Weber Syndrome, PCDH19-related epilepsy including PCDH19 pediatric epilepsy, Tuberous Sclerosis Complex, Dup15q Syndrome, Jeavons Syndrome, Febrile Illness-Related Epilepsy Syndrome (FIRES), Autoimmune Encephalitis/Encephalopathy due to Anti-LGI1, Anti-NMDAR, Anti-Gaba-B receptor, or Anti-GAD65 antibodies, FOXG1-related epilepsy, Syngapl-related epilepsy, Aicardi syndrome, sleep-related hypermotor epilepsy, epileptic encephalopathy with Continuous Spike and Wave During Sleep (CSWS), myoclonic epilepsy of infancy, Rasmussen's syndrome, developmental and epileptic encephalopathies (unclassified), focal epilepsy, juvenile absence epilepsy, posttraumatic epilepsy, epilepsy due to cortical dysplasia, and catamenial epilepsy, increased seizure activity, breakthrough seizures, and infantile spasms.

Epilepsy disorders and/or epilepsy related disorders that are suitable for treatment by the methods disclosed herein include CDKL5 Deficiency Disorder, Tuberous Sclerosis Complex, and PCDH19-related epilepsy. Another disorder that is suitable for treatment by the methods disclosed herein is Lennox-Gastaut syndrome (LGS).

The methods disclosed herein may also be used for treating other neurological disorders, such as anxiety, agitation, or depression (e.g., post-partum depression).

Additional medical conditions manifesting with seizures that can be treated with the methods disclosed herein include, but are not limited, to preeclampsia/eclampsia, drug-related seizures (including prescriptions drugs uses as prescribed, e.g., antihistamines, antibiotics, antidepressants, antipsychotics, and also illicit substances, e.g., PCP, cocaine, and amphetamines), drug withdrawal (such as withdrawal from THC, opioids, alcohol, or barbiturates), or antiepileptic drugs (AED) non-compliance.

Other disorders that may be treated by a method, compound, or composition disclosed herein are described in International Application Nos. PCT/US2006/045626, PCT/US2007/024606, PCT/US2010/045176, PCT/US2016/016977, PCT/US2016/057120, PCT/US2017/056565, PCT/US2018/028151, PCT/US2018/060037, PCT/US2019/064850, PCT/US2020/044843, PCT/US2020/063648, PCT/US2021/060459, and PCT/US2021/061937; U.S. Pat. No. 10,391,105; and U.S. Patent Publication No. 2019/0111059, each of which are incorporated herein by reference in their entireties.

The methods disclosed herein are suitable for treating seizures. For example, the methods disclosed herein are suitable for treating status epilepticus (SE). The methods disclosed herein are suitable for any form of SE. For example, early status epilepticus, established status epilepticus, refractory status epilepticus, super-refractory status epilepticus, non-convulsive status epilepticus (e.g., focal non-convulsive status epilepticus, complex partial non-convulsive status epilepticus, simple partial non-convulsive status epilepticus, subtle non-convulsive status epilepticus), generalized convulsive status epilepticus, complex partial status epilepticus; generalized periodic epileptiform discharges, and periodic lateralized epileptiform discharges. The method can also be used to treat subjects that have failed first-line treatment (e.g., benzodiazepine), second-line treatment (e.g. fosphenytoin, valproic acid or levetiracetam), and/or third-line treatment (thiopental, midazolam, pentobarbital, or propofol).

Other forms of seizures the methods disclosed herein can be suitable for treating include epileptic seizures, acute repetitive seizures, cluster seizures, continuous seizures, unremitting seizures, prolonged seizures, recurrent seizures, refractory seizures, myoclonic seizures, tonic seizures, tonic-clonic seizures, simple partial seizures, complex partial seizures, secondarily generalized seizures, atypical absence seizures, absence seizures, atonic seizures, benign Rolandic seizures, febrile seizures, emotional seizures, focal seizures, gelastic seizures, generalized onset seizures, infantile spasms, Jacksonian seizures, massive bilateral myoclonus seizures, multifocal seizures, neonatal onset seizures, nocturnal seizures, occipital lobe seizures, post traumatic seizures, subtle seizures, Sylvan seizures, visual reflex seizures, seizures from traumatic brain injury, seizures resulting from exposure to chemical weapons (e.g., organophosphate nerve gas or withdrawal seizures).

The compounds and pharmaceutical compositions disclosed herein can also be administered as a prophylactic for treating central nervous system disorders and/or neurological disorders. Exemplary central nervous system disorders and/or neurological disorders that may be suitable for treating using the formulations disclosed herein include autism spectrum disorders, Rett Syndrome, Tourette Syndrome, Obsessive Compulsive Disorder, insomnia, parasomnias, oppositional defiant disorder, conduct disorder, disruptive mood dysregulation disorder, agitation, anxiety, generalized anxiety disorder, social anxiety disorder, panic disorder, anxiety or agitation due to Alzheimer's dementia, schizophrenia, substance withdrawal syndrome (alcohol, benzodiazepine, barbiturate, and cocaine), post-traumatic stress disorder (PTSD), tremors, essential tremor, spasticity due to cerebral palsy, depression (including major depression, major depressive disorder, severe depression, unipolar depression, unipolar disorder, recurrent depression), postnatal or postpartum depression, atypical depression, melancholic depression, Psychotic Major Depression (PMD), catatonic depression, Seasonal Affective Disorder (SAD), dysthymia, double depression, Depressive Personality Disorder (DPD), Recurrent Brief Depression (RBD), minor depressive disorder, bipolar disorder or manic depressive disorder, post-traumatic stress disorders, post-menopausal depression, depression caused by chronic medical conditions, treatment-resistant depression, refractory depression, suicidality, suicidal ideation, mood disorder or suicidal behavior, attention deficit hyperactivity disorder, or disruptions in neurocognition including delirium. The compounds or compositions may also be used as a sedative agent or analgesic agent.

The disclosure further includes methods of treating seizures arising from neurodegenerative disorders. Such neurodegenerative disorders include Parkinson's disease, Alzheimer's disease, Amyotrophic Lateral Sclerosis, and Huntington's disease. The disclosure includes methods of treating seizure arising from inflammatory disorders, such as multiple sclerosis. The disclosure includes methods of treating seizure disorders arising from lysosomal storage disorders including Neimann-Pick disease type C (NPS, Tay-Sachs, Batten disease, Sandhoff disease, and Gaucher disease.

Although preferred subjects are human, typically any mammal including domestic animals such as dogs, cats and horses, may also be treated according to the methods disclosed herein.

Modified forms of ganaxolone intended as prodrugs are not predictably converted to ganaxolone. For example, conventional approaches to forming ganaxolone prodrugs have led to compounds that, while having improved aqueous solubility or oral absorption compared to ganaxolone, are not sufficiently converted to ganaxolone in vivo to provide therapeutically effective serum concentrations ganaxolone. Thus, there is a significant level of unpredictability in designing compounds suitable as ganaxolone prodrugs that are sufficiently stable (e.g., relatively bench stable, relatively stable in plasma, and relatively stable in gastric fluid and/or intestinal fluid), that have sufficient oral bioavailability, and can be converted to ganaxolone following administration to provide therapeutic serum concentrations of ganaxolone. Thus, it is both surprising and unexpected that certain compounds disclosed herein are sufficiently stable, have improved solubility and other physicochemical properties linked to bioavailability, and are also efficiently converted to ganaxolone by metabolic processes (see, e.g., Example 121). Even minor modifications to a compound structure was found to significantly reduce or eliminate the ability of the compound to be converted to ganaxolone, or negatively affect its stability, which demonstrates the high degree of unpredictability and difficulty in designing useful ganaxolone prodrugs.

### Methods of Synthesis

The compounds disclosed herein can be prepared according to known processes. Schemes 1-3 below represent general synthetic schemes for preparing compounds of the present disclosure (e.g., compounds of Formulae (I), (I-I), (IA), (IA-a), (IA-b), (IB), (IC), (ID), (IE), (I-II), (I-IIa), (I-IIb), (I-IIc), (I-IId), (I-IIe), and compounds of Table 1). These schemes are illustrative and are not meant to limit the possible techniques one skilled in the art may use to prepare compounds disclosed herein. Different methods will be evident to those skilled in the art. Various modifications to these methods may be envisioned by those skilled in the art to achieve similar results to those described below. Optional protecting groups can be used as described, for example, in Greene et al. Protective Groups in Organic Synthesis (4^{th} ed. 2006). It will be appreciated that one or more additional steps of protecting and/or deprotecting, e.g., a nitrogen or oxygen atom, saponification, and/or a step of forming a salt (e.g., by treatment with an acid) may be involved in the syntheses that are not depicted in the generalized schemes for clarity.

A compound of Formula (I) can generally be prepared, for example, according to Scheme 1:

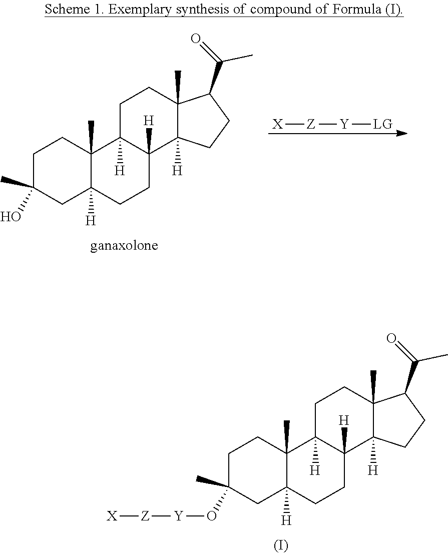

wherein X, Z, and Y are as defined herein, and LG is a suitable leaving group (e.g., halogen, OSO₂Me, OMs, OTs, OTf, or OH). The reaction may be catalyzed by heat and/or a reagent (e.g., acid or base) or catalyst, and can be carried out in any suitable solvent, such as an inert organic solvent.

A compound of Formula (I-I) can generally be prepared, for example, according to Scheme 2:

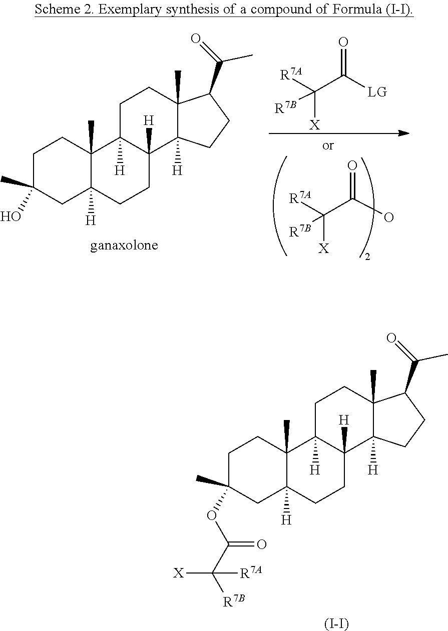

wherein X, R^{7A} and R^{7B} are as defined herein, and LG is a suitable leaving group (e.g., halogen, OSO₂Me, OMs, OTs, OTf, or OH). The reaction may be catalyzed by heat and/or a reagent (e.g., acid or base) or catalyst, and can be carried out in any suitable solvent, such as an inert organic solvent.

Alternatively, a compound of Formula (I-I) can generally be prepared, for example, according to Scheme 3:

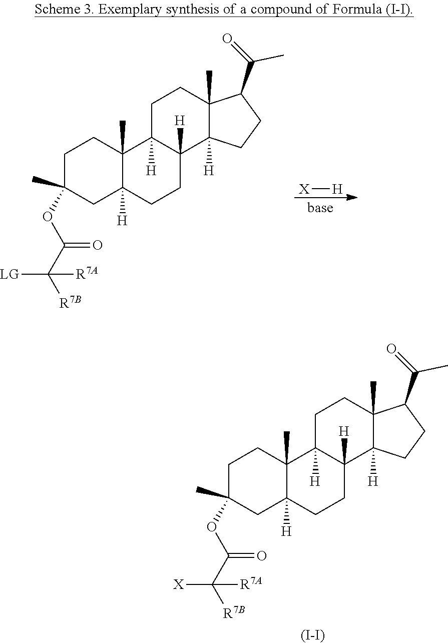

wherein X, R^{7A} and R^{7B} are as defined herein, and LG is a suitable leaving group (e.g., halogen, OSO₂Me, OMs, OTs, OTf, or OH). The reaction may be catalyzed by heat and/or a reagent (e.g., acid or base) or catalyst, and can be carried out in any suitable solvent, such as an inert organic solvent.

A base used in the synthesis of a compound disclosed herein, e.g., according to one of Schemes 1-3, may include an inorganic base such as potassium tert-butoxide, sodium hydride, sodium methoxide, sodium ethoxide, sodium hydroxide, potassium hydroxide, or the like, or may include an organic base such as an amine base (e.g., triethylamine (TEA), diisopropylamine, diisopropylethylamine (DIPEA), pyrrolidine, or pyridine).

An acid used in the synthesis of a compound disclosed herein, e.g., according to one of Schemes 1-3, may include hydrochloric acid, sulfuric acid, nitric acid, tosic acid, trifluoroacetic acid, acetic acid, or the like.

A solvent used in the synthesis of a compound disclosed herein, e.g., according to one of Schemes 1-3, may include dimethylformamide (DMF), N-methyl-2-pyrrolidone (NMP), tetrahydrofuran (THF), dimethyl sulfoxide (DMSO), dichloromethane (DCM), 1,4-dioxane, acetonitrile, methanol, ethanol, toluene, diethyl ether, methyl tert-butyl ether (MTBE), or the like. Water may also be used as a solvent, either alone or as a mixture with one or more other solvents, e.g., a solvent described herein.

A reaction to prepare a compound disclosed herein, e.g., according to one of Schemes 1-3, may be carried out at any suitable temperature. For example, a reaction to prepare a compound disclosed herein may be carried out at room temperature, below room temperature, or above room temperature. A reaction to prepare a compound disclosed herein may be carried out at 0° C. or less, e.g., −5° C., −10° C., −25° C., −50° C., −78° C., −80° C., −100° C., or less. A reaction to prepare a compound disclosed herein may be carried out at 25° C. or more, e.g., 30° C., 35° C., 40° C., 45° C., 50° C., 60° C., 70° C., 80° C., 90° C., 100° C., 110° C., 120° C., or more.

A reaction described herein, e.g., according to one of Schemes 1-3, can be subsequently followed by further separation and purification steps, such as chromatography (e.g., flash column chromatography, high-performance liquid chromatography (HPLC), ultra-performance liquid chromatography (UPLC), etc.), crystallization, trituration, filtration, lyophilization, and the like.

### EQUIVALENTS

It will be readily apparent to those skilled in the art that other suitable modifications and adaptions of the compounds, compositions, kits, and methods of the present disclosure are obvious and may be made using suitable equivalents without departing from the scope of the disclosure or the embodiments. Having now described certain compounds, compositions, kits, and methods in detail, the same will be more clearly understood by reference to the following examples, which are introduced for illustration only and not intended to be limiting.

### EXAMPLES

### Materials and Methods

Unless otherwise noted, all starting materials were obtained from commercial suppliers and used without purification.

### Preparative and Analytical Methods

LC/MS data (ESI+) were determined with a Waters Alliance 2695 IPLC/MS (Waters Symmetry C18, 4.6×75 mm, 3.5 m) or (Phenomenex C18, 4.6×75 mm, 3.0 m) with a 2996 diode array detector from 210-400 nm; the solvent system is 5-95% acetonitrile (MeCN) in water (with 0.1% trifluoroacetic acid (TFA)) over nine minutes using a linear gradient, and retention times are in minutes. Mass spectrometry was performed on a Waters ZQ using electrospray in positive mode.

LC/MS data (ESI−) were determined with a Shimadzu Prominence HPLC/MS (Phenomenex Luna C18, 3.0×50 mm, 3 m) with a 2996 diode array detector from 210-400 nm; the solvent system is 5-95% MeCN in water (with 0.1% formic acid) over five minutes using a linear gradient, and retention times are in minutes. Mass spectrometry was performed on a Applied Biosystems MDS Sciex API 2000 using electrospray in negative mode. Alternatively LC/MS data (ESI−) were determined with a Waters Alliance 2695 HPLC/MS (Phenomenex C18, 4.6×75 mm, 3.0 μm) with a 2996 diode array detector from 210-400 nm; the solvent system is 5-95% MeCN in water (with 0.1% formic acid) over nine minutes using a linear gradient, and retention times are in minutes. Mass spectrometry was performed on a Waters ZQ using electrospray in positive mode.

HRMS data were determined by The University of Notre Dame Mass Spectrometry & Proteomics Facility on a Bruker micrOTOF II.

Preparative reversed phase HPLC was performed on a Waters Sunfire column (19×50 mm, C18, 5 μm) with a 10 min mobile phase gradient of 10% acetonitrile/water to 90% acetonitrile/water, with 0.1% TFA as buffer, using 214 and 254 nm as detection wavelengths. Injection and fraction collection were performed with a Gilson 215 liquid handling apparatus using Trilution LC software.

NMR was recorded with a Varian Oxford 300 MHz, in CDCl3, unless otherwise noted. Chemical shifts (δ) are expressed in ppm downfield from tetramethylsilane (TMS).

Purities are generally ≥95% by NMR or LCMS except otherwise noted.

### Example 1. Synthesis of Compound 404

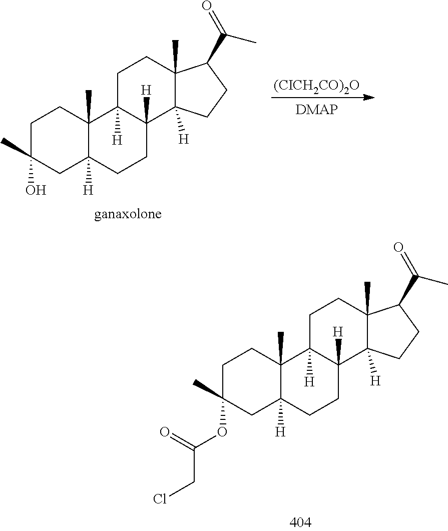

To a 250 mL 3-neck flask equipped with an overhead stirrer, drying tube and addition funnel was charged ganaxolone (1.01 g, 3.04 mmol) and 30 mL of 1,2-dichloroethane to produce a thick slurry. To this was charged DMAP (0.74 g, 6.04 mmol), which clarified the reaction mixture. To this was charged Et₃N (2.55 mL, 13.28 mmol), producing a clear solution which was stirred at ambient temperature for 15 min. A solution of chloroacetic anhydride (5.18 g, 30.3 mmol) dissolved in 35 mL of 1,2-DCE was added via an addition funnel over the course of 15 min. Upon the addition, the reaction instantly became a neon yellow solution, then a slurry and finally a dark brown solution. The reaction was allowed to stir for 1 h and then was quenched with 100 mL of 1M HCl. This mixture was allowed to stirred for 5 minutes. The phases were separated and the lower organic was dried with Na₂SO₄ for ˜30 minutes, concentrated and dried on high vacuum pump to a dark semi solid material. The solid was dissolved in 10 mL of DCM and then concentrated onto silica gel. The resulting brown solid was dried under high vacuum for 30 minutes. A 20-gram silica cartridge was then prepped for use, a gradient of 0-5% EtOAc/hexanes was run over 10 volumes, followed by a hold at 5% for 15 volumes. After the hold, the EtOAc was ramped to 100%, then held for 5 volumes. Concentration of the product containing fractions provided compound 404 as a solid (1.1439 g). HRMS (ESI+): calculated for C₂₄H₃₈ClO₃ m/z [M+H]⁺: 409.2504, observed 409.2517; ¹H NMR 300 MHz DMSO, d6 δ: 3.98-4.05 (s, 2H), 2.47-2.58 (m, 1H), 2.09-2.30 (m, 4H), 1.89-2.06 (m, 2H), 1.47-1.77 (m, 10H), 1.90-1.46 (m, 10H), 0.69-1.03 (m, 5H), 0.57-0.67 (m, 3H).

### Example 2. Synthesis of Compound 100

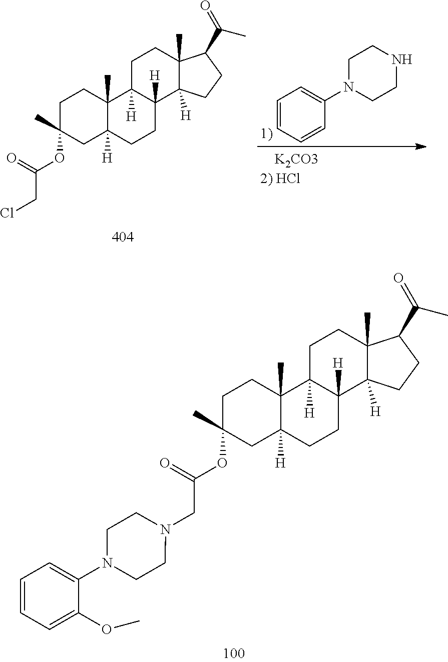

To a solution of 1-(2-methoxyphenyl)-piperazine (12.9 mg, 0.07 mmol) and K₂CO₃ (10 mg, 0.07 mmol) in MeCN (0.55 mL) was added Compound 404 (25 mg, 0.06 mmol), and the contents were stirred and heated to 50° C. overnight. Once the reaction was deemed complete by TLC, 0.5 M HCl (15 mL) was added, followed by ethyl acetate (15 mL). The organic layer was separated, washed with water (15 mL) and brine (15 mL), and dried with Na₂SO₄. The organic layer was decanted, and solvent was removed in vacuo. The crude product was purified by column chromatography using an ISCO™ chromatography system. The resulting free base was treated with 2.0 M HCl/diethyl ether (2 mL), and solvent was removed in vacuo to provide compound 100 as a solid (44.7 mg). LCMS (ESI+): RT=5.55 min, (M+H)+=565.55; HRMS (ESI+): calculated for C₃₅H₅₃N₂O₄ m/z [M+H]⁺: 565.4000, observed 565.4003; ¹H NMR 300 MHz CD₃OD δ: 6.87-7.13 (m, 4H), 4.21 (s, 2H), 3.85 (s, 3H), 3.44-3.67 (m, 4H), 3.14-3.39 (m, 4H, coincident with solvent), 2.54-2.63 (br. s, 1H), 2.10-2.30 (m, 2H), 2.09 (s, 3H), 1.84-2.07 (m, 2H), 1.57-1.77 (m, 5H), 1.55 (s, 3H), 1.09-1.52 (m, 11H), 0.85-1.06 (m, 1H), 0.83 (s, 3H), 0.74 (m, 1H), 0.59 (s, 3H).

### Example 3. Synthesis of Compound 101

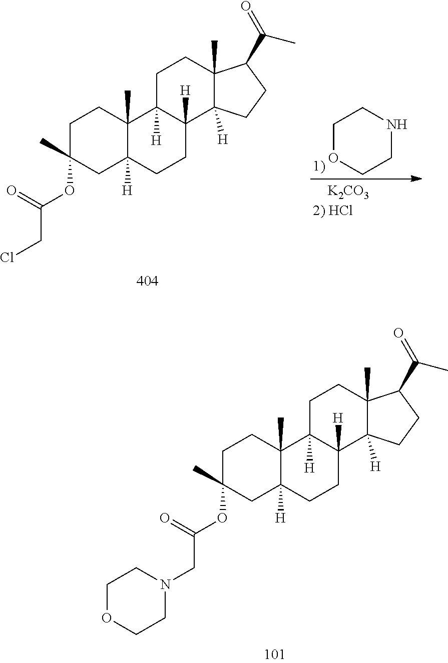

In a similar manner to the procedure outlined in Example 2, substituting 1-(2-methoxyphenyl)-piperazine with morpholine, Compound 101 was obtained as an HCl salt. LCMS (ESI) m/z (M+1)+460.36; HRMS (ESI+) calculated for C₂₈H₄₆NO₄ m/z [M+H]⁺: 460.3421, observed 460.3416; ¹H NMR δ: 4.03-4.22 (br. s, 4H), 3.77 (s, 2H), 3.38-3.52 (br. s, 4H), 2.48-2.57 (br. s, 1H), 2.15-2.29 (m, 2H), 2.11 (s, 3H), 1.80-2.06 (m, 2H), 1.54-1.75 (m, 5H), 1.53 (s, 3H), 1.12-1.49 (m, 11H), 0.82-1.11 (m, 1H), 0.77 (s, 3H), 0.69-0.75 (m, 1H), 0.60 (s, 3H).

### Example 4. Synthesis of Compound 102

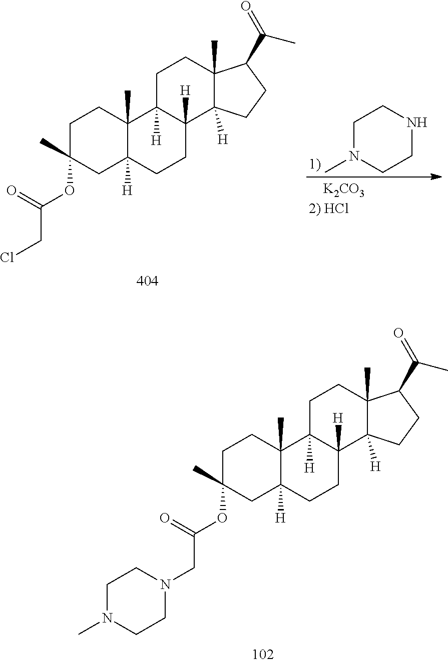

In a similar manner to the procedure outlined in Example 2, substituting 1-(2-methoxyphenyl)-piperazine with methylpiperazine, Compound 102 was obtained as an HCl salt. LCMS (ESI) m/z (M+1)+473.39; HRMS (ESI+) calculated for C₂₉H₄₉N₂O₃ m/z [M+H]+: 473.3738, observed 473.3732; ¹H NMR (CD₃OD) δ: 3.83 (s, 2H), 3.12-3.62 (m, 8H, coincident with solvent), 2.94 (s, 3H), 2.54-2.62 (br. s, 1H), 2.10-2.28 (m, 2H), 2.08 (s, 3H), 1.82-2.05 (m, 2H), 1.52-1.75 (m, 5H), 1.50 (s, 3H), 1.08-1.48 (m, 11H), 0.83-1.06 (m, 1H), 0.81 (s, 3H), 0.68-0.79 (m, 1H), 0.58 (s, 3H).

### Example 5. Synthesis of Compound 103

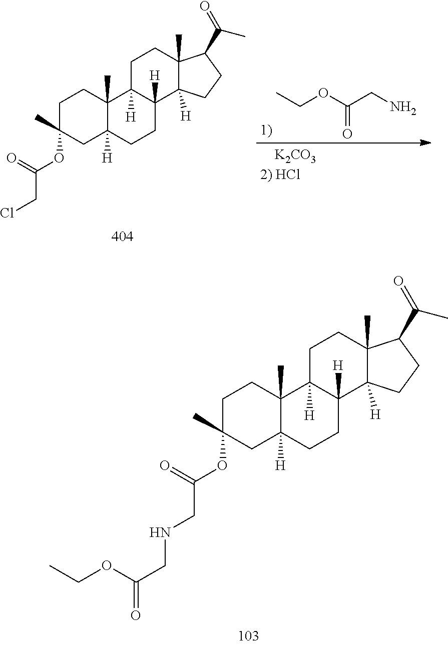

In a similar manner to the procedure outlined in Example 2, substituting 1-(2-methoxyphenyl)-piperazine with glycine ethyl ester, Compound 103 was obtained as an HCl salt. LCMS (ESI) m/z (M+1)+476.45; HRMS (ESI+) calculated for C₂₈H₄₆NO₅ m/z [M+H]⁺: 476.3370, observed 476.3362; ¹H NMR CD₃OD δ: 4.30 (q, J=7.4 Hz, 2H), 4.00 (s, 2H), 3.98 (s, 2H), 2.59 (t, J=8.4 Hz, 1H), 2.10-2.25 (m, 2H), 2.09 (s, 3H), 1.85-2.06 (m, 2H), 1.54-1.76 (m, 5H), 1.52 (s, 3H), 1.08-1.50 (m, 14H), 0.84-1.02 (m, 1H), 0.82 (s, 3H), 0.64-0.80 (m, 1H), 0.59 (s, 3H).

### Example 6. Synthesis of Compound 104

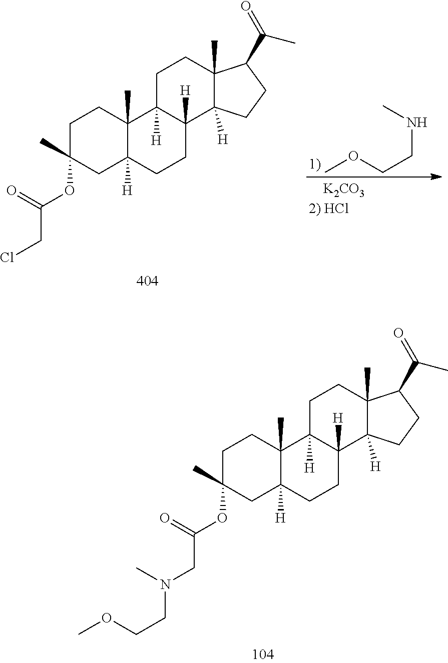

In a similar manner to the procedure outlined in Example 2, substituting 1-(2-methoxyphenyl)-piperazine with N-(2-methoxyethyl)methylamine, Compound 104 was obtained as an HCl salt. LCMS (ESI) m/z (M+1)⁺ 462.36; HRMS (ESI+) calculated for C₂₈H₄₈NO₄ m/z [M+H]⁺: 462.3578, observed 462.3566; ¹H NMR CD₃OD δ: 4.09-4.23 (br. s., 2H), 3.75 (t, J=5.0 Hz, 2H), 3.43-3.54 (br. s, 2H), 3.41 (s, 3H), 2.99 (s, 3H), 2.60 (t, J=9.08 Hz, 1H), 2.12-2.29 (m, 2H), 2.10 (s, 3H), 1.85-2.08 (m, 2H), 1.55-1.79 (m, 5H), 1.54 (s, 3H), 1.11-1.53 (m, 11H), 0.86-1.05 (m, 1H), 0.84 (s, 3H), 0.65-0.82 (m, 1H), 0.61 (s, 3H).

### Example 7. Synthesis of Compound 105

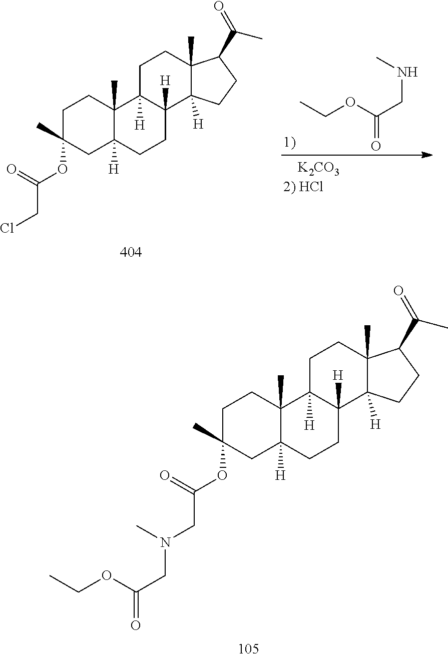

In a similar manner to the procedure outlined in Example 2, substituting 1-(2-methoxyphenyl)-piperazine with sarcosine ethyl ester, Compound 105 was obtained as an HCl salt. LCMS (ESI) m/z (M+1)⁺490.41; ¹H NMR CD₃OD δ: 4.33 (q, J=7.0 Hz, 2H), 4.20 (s, 4H), 3.05 (s, 3H), 2.60 (t, J=8.6 Hz, 1H), 2.12-2.29 (m, 2H), 2.10 (s, 3H), 1.87-2.08 (m, 2H), 1.56-1.82 (m, 5H), 1.55 (s, 3H), 1.13-1.54 (m, 14H), 0.86-1.07 (m, 1H), 0.84 (s, 3H), 0.69-0.83 (m, 1H), 0.60 (s, 3H).

### Example 8. Synthesis of Compound 106

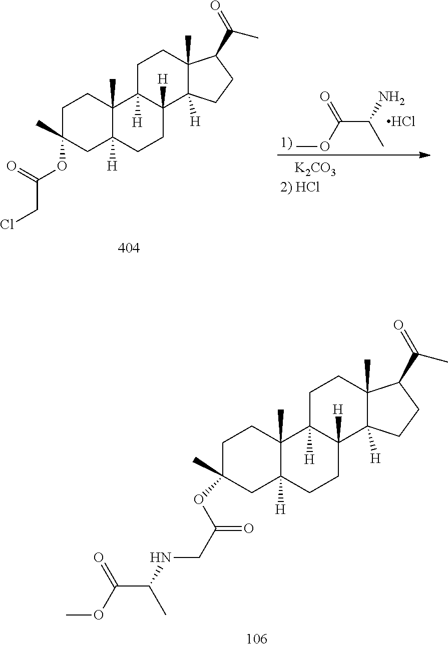

In a similar manner to the procedure outlined in Example 2, substituting 1-(2-methoxyphenyl)-piperazine with D-alanine methyl ester hydrochloride, Compound 106 was obtained as an HCl salt. LCMS (ESI) m/z (M+1)⁺476.31; HRMS (ESI+) calculated for C₂₈H₄₆NO₅ m/z [M+H]⁺: 476.3370, observed 476.3376; ¹H NMR CD₃OD δ: 4.15 (q, J=7.0 Hz, 1H), 3.98 (d, J=2.3 Hz, 2H), 3.86 (s, 3H), 2.60 (t, J=8.8 Hz, 1H), 2.12-2.28 (m, 2H), 2.10 (s, 3H), 1.87-2.08 (m, 2H), 1.55-1.78 (m, 8H), 1.54 (s, 3H), 1.12-1.53 (m, 11H), 0.87-1.06 (m, 1H), 0.84 (s, 3H), 0.74-0.82 (m, 1H), 0.60 (s, 3H).

### Example 9. Synthesis of Compound 107

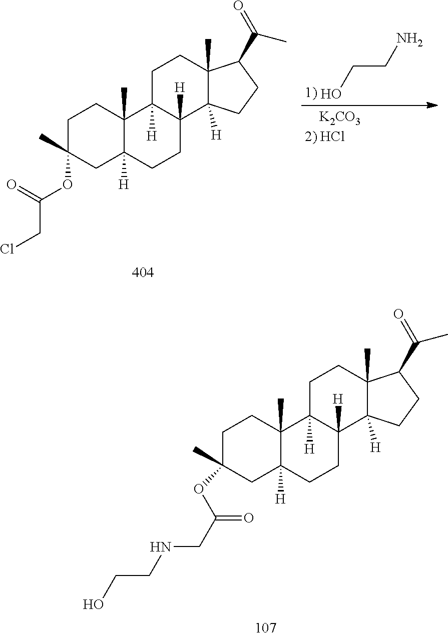

In a similar manner to the procedure outlined in Example 2, substituting 1-(2-methoxyphenyl)-piperazine with aminoethanol, Compound 107 was obtained as an HCl salt. LCMS (ESI) m/z (M+1)⁺434.16; HRMS (ESI+) calculated for C₂₆H₄₄NO₄ m/z [M+H]⁺: 434.3265, observed 434.3258; ¹H NMR CD₃OD δ: 3.95 (s, 2H), 3.81 (t, J=5.2 Hz, 2H), 3.18 (t, J=5.0 Hz, 2H), 2.60 (t, J=8.8 Hz, 1H), 2.12-2.28 (m, 2H), 2.10 (s, 3H), 1.88-2.08 (m, 2H), 1.55-1.77 (m, 5H), 1.53 (s, 3H), 1.11-1.52 (m, 11H), 0.88-1.06 (m, 1H), 0.84 (s, 3H), 0.74-0.82 (m, 1H), 0.60 (s, 3H).

### Example 10. Synthesis of Compound 108

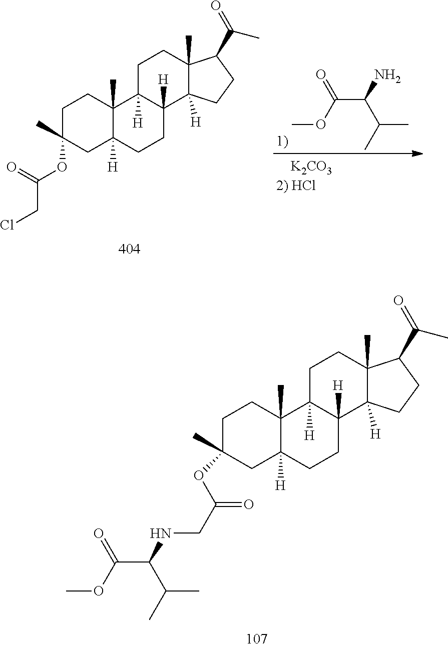

In a similar manner to the procedure outlined in Example 2, substituting 1-(2-methoxyphenyl)-piperazine with L-valine methyl ester, Compound 108 was obtained as an HCl salt. LCMS (ESI) m/z (M+1)⁺504.30; HRMS (ESI+) calculated for C₃₀H₅₀NO₅ m/z [M+H]⁺: 504.3684, observed 504.3695; ¹H NMR CD₃OD δ: 4.03 (d, J=3.5 Hz, 1H), 3.98 (s, 2H), 3.88 (s, 3H), 2.61 (t, J=8.8 Hz, 1H), 2.31-2.46 (m, 1H), 2.13-2.30 (m, 2H), 2.11 (s, 3H), 1.86-2.09 (m, 2H), 1.55-1.79 (m, 5H), 1.54 (s, 3H), 1.17-1.53 (m, 11H), 1.14 (d, J=7.0 Hz, 3H), 1.07 (d, J=7.0 Hz, 3H), 0.87-1.02 (m, 1H), 0.84 (s, 3H), 0.65-0.83 (m, 1H), 0.60 (s, 3H).

### Example 11. Synthesis of Compound 109

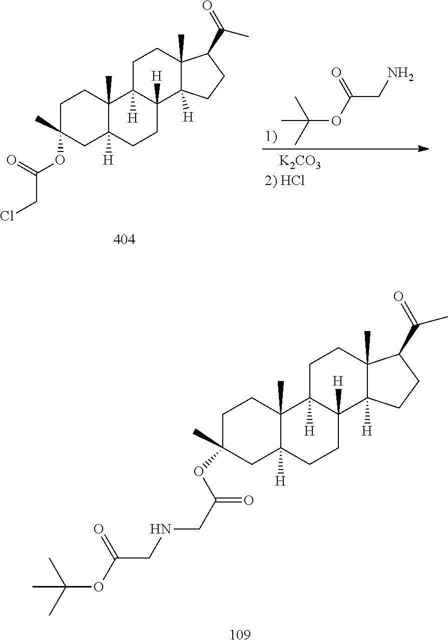

In a similar manner to the procedure outlined in Example 2, substituting 1-(2-methoxyphenyl)-piperazine with glycine tert-butyl ester, Compound 109 was obtained as an HCl salt. LCMS (ESI) m/z (M+1)⁺504.23; HRMS (ESI+) calculated for C₃₀H₅₀NO₅ m/z [M+H]⁺: 504.3684, observed 504.3673; ¹H NMR δ: 3.39-3.50 (m, 4H), 2.75-2.90 (br. s, 1H), 2.51 (t, J=9.1 Hz, 1H), 2.11-2.26 (m, 2H), 2.10 (s, 3H), 1.82-2.09 (m, 2H), 1.51-1.73 (m, 5H), 1.49 (s, 3H), 1.48 (s, 9H), 1.04-1.46 (m, 11H), 0.82-1.02 (m, 1H), 0.76 (s, 3H), 0.64-0.75 (m, 1H), 0.59 (s, 3H).

### Example 12. Synthesis of Compound 110

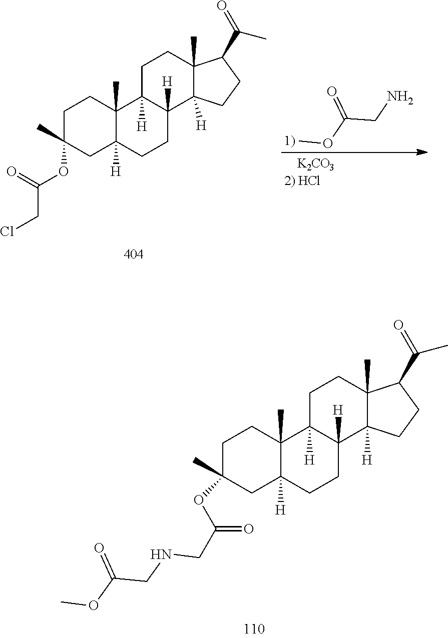

In a similar manner to the procedure outlined in Example 2, substituting 1-(2-methoxyphenyl)-piperazine with glycine methyl ester, Compound 110 was obtained as an HCl salt. LCMS (ESI) m/z (M+1)⁺462.29; HRMS (ESI+) calculated for C₂₇H₄₄NO₅ m/z [M+H]⁺: 462.3214, observed 462.3207; ¹H NMR δ: 4.03 (s, 2H), 4.00 (s, 2H), 3.86 (s, 3H), 2.61 (t, J=8.8 Hz, 1H), 2.13-2.28 (m, 2H), 2.11 (s, 3H), 1.88-2.08 (m, 2H), 1.56-1.80 (m, 5H), 1.54 (s, 3H), 1.14-1.54 (m, 11H), 0.87-1.07 (m, 1H), 0.84 (m, 3H), 0.72-0.82 (m, 1H), 0.60 (s, 3H).

### Example 13. Synthesis of Compound 300

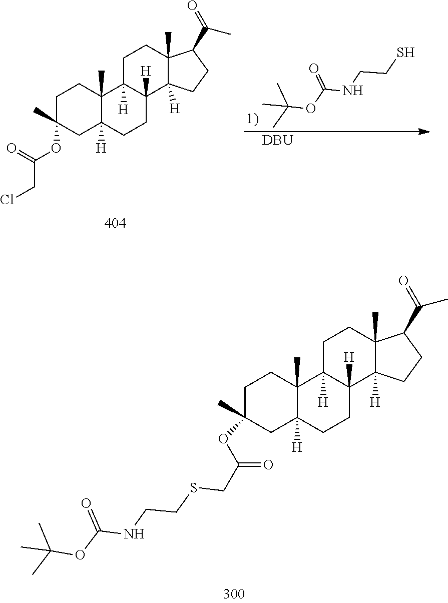

To a solution of BOC-cysteamine (23.8 mg, 0.13 mmol), 1,8-diazabicyclo[5.4.0]undec-7-ene (20.5 mg, 0.13 mmol), and NaI (9.2 mg, 0.06 mmol) in MeCN (2 mL) was added compound 404 (50 mg, 0.12 mmol). The contents were stirred at ambient temperature overnight. Once the reaction was deemed complete by TLC, a 1:1 mixture (7 mL) of saturated aqueous NH₄Cl and water was added, followed by dichloromethane (DCM) (9 mL). The organic layer was separated, washed with 1:1 NH₄Cl:water (9 mL), dried over Na₂SO₄, and filtered through a phase separator column. The aqueous phase from the extraction was washed with DCM (9 mL), which was then separated, dried over Na₂SO₄, and filtered through the aforementioned phase separator column. The organic layers were combined, and solvent was removed in vacuo. The crude product was purified by column chromatography using an ISCO™ chromatography system to provide compound 300 as an oil (61.6 mg). LCMS (ESI+): RT=7.91 min, (M+H)⁺=550.45; HRMS (ESI+): calculated for C₃₁H₅₁NNaO₅S m/z [M+H]⁺: 572.3380, observed 572.3371; ¹H NMR 300 MHz δ: 4.92-5.05 (br. s, 1H), 3.28-3.50 (m, 2H), 3.18 (s, 2H), 2.77 (t, J=6.1, 2H), 2.53 (t, J=8.8, 1H), 2.13-2.25 (m, 2H), 2.11 (s, 3H), 1.88-2.03 (m, 2H), 1.49-1.74 (m, 5H), 1.48 (s, 3H), 1.45 (s, 9H), 1.08-1.43 (m, 11H), 0.83-1.01 (m, 1H), 0.77 (s, 3H), 0.68-0.75 (m, 1H), 0.60 (s, 3H).

### Example 14. Synthesis of Compound 301

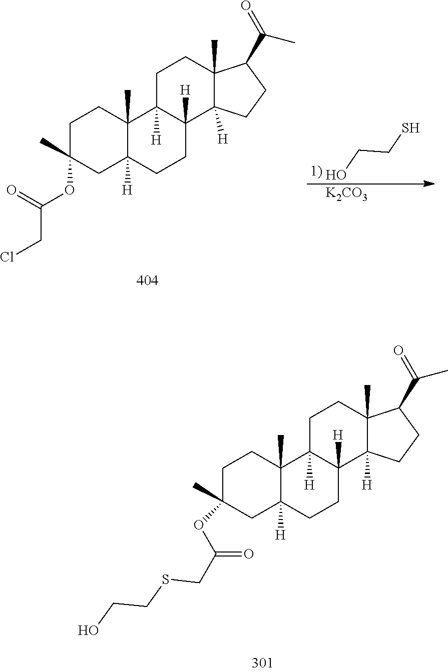

To a solution of 2-mercaptoethanol (57.3 mg, 0.73 mmol) and K₂CO₃ (101 mg, 0.73 mmol) in MeCN (2 mL) was added compound 404 (200 mg, 0.49 mmol). The contents were stirred and heated to 50° C. overnight. Once the reaction was deemed complete by TLC, a 1:1 mixture (7 mL) of saturated aqueous NH₄Cl and water was added, followed by DCM (9 mL). The organic layer was separated, washed with 1:1 NH₄Cl:water (9 mL), dried over Na₂SO₄, and filtered through a phase separator column. The aqueous phase from the extraction was washed with DCM (9 mL), which was then separated, dried over Na₂SO₄, and filtered through the aforementioned phase separator column. The organic layers were combined, and solvent was removed in vacuo. The crude product was purified by column chromatography using an ISCO™ chromatography system to provide compound 301 as a solid (177 mg). LCMS (ESI+): RT=6.86 min, (M+H)+=451.25; HRMS (ESI+): calculated for C26H42NaO4S m/z [M+H]⁺: 473.2696, observed 473.2692; ¹H NMR 300 MHz δ: 3.78 (t, J=5.5 Hz, 2H), 3.22 (s, 2H), 2.84 (t, J=5.5 Hz, 2H), 2.52 (t, J=8.8 Hz, 1H), 2.13-2.24 (m, 2H), 2.11 (s, 3H), 1.90-2.07 (m, 2H), 1.51-1.73 (m, 6H), 1.48 (s, 3H), 1.09-1.47 (m, 11H), 0.83-1.00 (m, 1H), 0.77 (s, 3H), 0.66-0.76 (m, 1H), 0.60 (s, 3H).

### Example 15. Synthesis of Compound 302

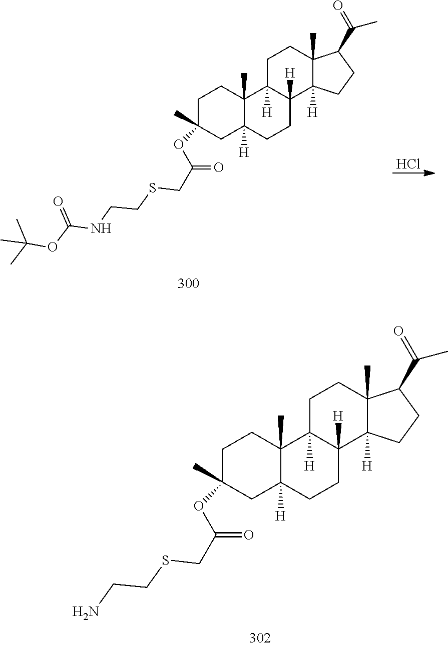

A solution of compound 300 (43.2 mg, 0.08 mmol) in 4 N HCl/dioxane (1 mL) was vigorously stirred at ambient temperature for 10 min. The mixture was diluted with DCM (9 mL), followed by water (8 mL). The organic layer was separated, washed with brine (9 mL), dried over Na₂SO₄, and filtered through a phase separator column. The aqueous layer was washed with DCM (9 mL), which was then separated, dried over Na₂SO₄, and filtered through the aforementioned phase separator column. The organic layers were combined, and solvent was removed in vacuo, followed by lyophilization to remove residual dioxane. Compound 302 was isolated as a solid (HCl salt; 30.7 mg). LCMS (ESI+): RT=5.64 min, (M+H)⁺=450.25; HRMS (ESI+): calculated for C₂₆H₄₄NO₃S m/z [M+H]⁺: 450.3036, observed 450.3042; ¹H NMR 300 MHz CD3OD δ: 3.33 (s, 2H), 3.15 (t, J=6.7 Hz, 2H), 2.91 (t, J=6.7 Hz, 2H), 2.61 (t, J=9.1 Hz, 1H), 2.13-2.24 (m, 2H), 2.11 (s, 3H), 1.87-2.08 (m, 2H), 1.49-1.83 (m, 5H), 1.48 (s, 3H), 1.13-1.48 (m, 11H), 0.86-1.06 (m, 1H), 0.83 (s, 3H), 0.63-0.82 (m, 1H), 0.60 (s, 3H).

### Example 16. Preparation of Compound 303

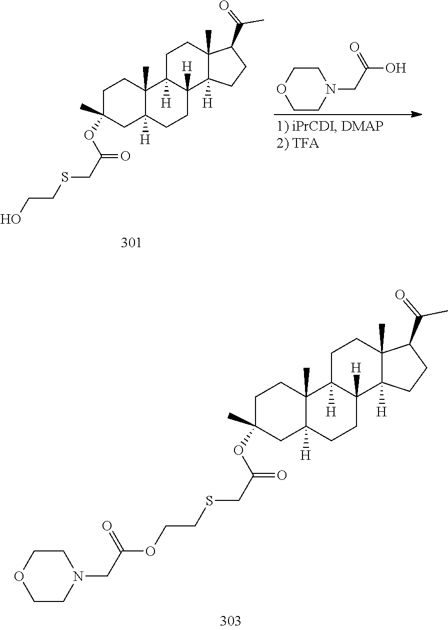

To a solution of morpholin-4-yl-acetic acid (184 mg, 1.3 mmol), N,N′-diisopropylcarbodiimide (479 mg, 3.8 mmol), and 4-(dimethylamino)pyridine (464 mg, 3.8 mmol) in DCM (4 mL) was added compound 301 (190 mg, 0.42 mmol). The contents were stirred at ambient temperature overnight. Once the reaction was deemed complete by TLC, a 1:1 mixture (18 mL) of saturated aqueous NH₄Cl and water was added, followed by DCM (14 mL). The organic layer was separated, washed with 1:1 NH₄Cl:water (18 mL), dried over Na₂SO₄, and filtered through a phase separator column. The aqueous phase from the extraction was washed with DCM (18 mL), which was then separated, dried over Na₂SO₄, and filtered through the aforementioned phase separator column. The organic layers were combined, and solvent was removed in vacuo. The crude product was purified by column chromatography using an ISCO™ chromatography system, followed by further purification using a Gilson™ Semi Prep HPLC system to provide compound 303 as a solid (TFA salt; 166 mg). LCMS (ESI+): RT=5.51 min, (M+H)⁺=578.34; HRMS (ESI+): calculated for C₃₂H₅₂NO₆S m/z [M+H]⁺: 578.3510, observed 578.3504; ¹H NMR 300 MHz CD30D δ: 4.48 (t, J=6.5 Hz, 2H), 4.24 (s, 2H), 3.88-4.00 (br. s, 4H), 3.35-3.50 (br. s, 4H), 3.27 (s, 2H), 2.96 (t, J=4.7, 2H), 2.61 (t, J=9.1 Hz, 1H), 2.12-2.22 (m, 2H), 2.11 (s, 3H), 1.88-2.07 (m, 2H), 1.48-1.77 (m, 5H), 1.47 (s, 3H), 1.15-1.45 (m, 11H), 0.86-1.05 (m, 1H), 0.83 (s, 3H), 0.72-0.81 (m, 1H), 0.60 (s, 3H).

### Example 17: Synthesis of Compound 200

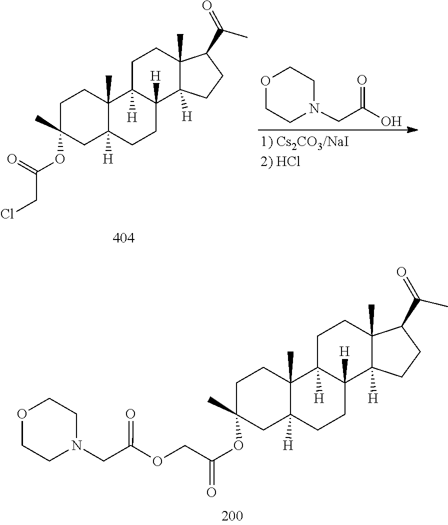

Anhydrous DMF (3 mL) was added to compound 404 (75.6 mg, 0.18 mmol), 4-morpholineacetic acid (125 mg, 0.95 mmol) and sodium iodide (46.7 mg, 0.31 mmol) to produce a clear orange solution. The solution was stirred ambient temperature for 15 minutes. After 15 mins, Cs₂CO₃ (292 mg, 0.90 mmol) was added and the resulting mixture was heated to 70° C. and allowed to stir overnight. The reaction mixture was cooled and diluted with 25 mL of EtOAc, and then quenched with 15 mL of water. The layers were separated and the EtOAc layer was washed with 2×15 ml of saturated NaHCO₃ solution, dried with Na₂SO₄ for 30 minutes, filtered and concentrated to provide a clear orange oil. The crude mixture was purified by column chromatography. The oil was dissolved in DCM and stripped on to silica gel. The silica gel was dried under high vacuum for 15 minutes, then loaded onto a 4-gram cartridge and eluted with EtOAc in hexanes running isocratic at 25% EtOAc, then ramping to 50%, followed by ramping to 100% EtOAc. The compound 200 (free base; 81.1 mg) was obtained as an oil. HRMS (ESI+): calculated for C30H48NO6 m/z [M+H]⁺: 518.3476, observed 518.3484; ¹H NMR 300 MHz δ: 4.50-4.68 (m, 2H), 3.71-3.83 (m, 4H), 3.29-3.38 (m, 2H), 2.58-2.73 (m, 4H), 2.45-2.57 (m, 1H), 2.06-2.30 (m, 6H), 1.94-2.05 (m, 1H), 1.86 (dd, J=11.14 Hz, 2.34 Hz, 1H), 1.05-1.76 (m, 19H), 0.71-1.03 (m, 4H), 0.57-0.65 (m, 3H).

Compound 200 (free base; 42.4 mg, 0.08 mmol) was dissolved in 2 mL of Et₂O to produce a clear solution and stirred for 5 minutes. 2M HCl in Et₂O (50 μL, 0.1 mmol) was added and a white precipitate formed. The reaction was allowed to stir for 10 minutes. The precipitate was isolated by filtration and washed with 5 mL of Et₂O. The solid was then placed under high vacuum to dry overnight to afford compound 200 HCl salt. HRMS (ESI+): calculated for C₃₀H₄₈NO₆ m/z [M+H]⁺: 518.3476, observed 518.3484; ¹H NMR 300 MHz δ: 4.58-4.81 (m, 2H), 4.02-4.36 (m, 3H), 3.94 (br. s., 2H), 3.40-3.61 (m, 4H), 2.53 (t, J=8.76 Hz, 1H), 2.08-2.28 (m, 5H), 1.95-2.07 (m, 1H), 1.86 (d, J=8.79 Hz, 1H), 1.08-1.78 (m, 19H), 0.74-1.04 (m, 4H), 0.59-0.73 (m, 4H).

### Example 18: Synthesis of Compound 202

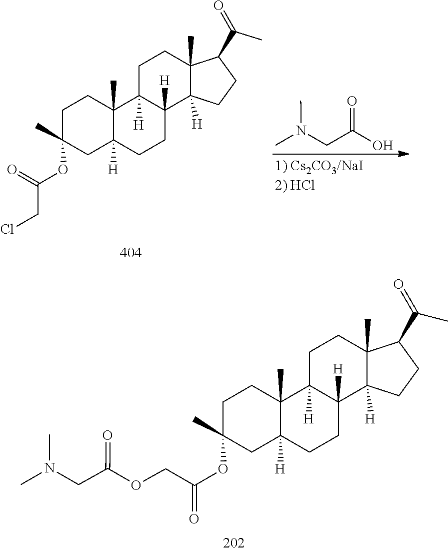

In a similar manner to the procedure outlined in Example 17, substituting 4-morpholineacetic acid with dimethyl glycine, Compound 202 was obtained as an HCl salt. HRMS (ESI+) calculated for C₂₈H₄₆NO₅ m/z [M+H]⁺: 476.3370, observed 476.3372; ¹H NMR δ: Free base) δ: 4.45-4.75 (m, 2H), 3.29 (s, 2H), 2.28-2.72 (m, 4H), 1.76-2.28 (m, 5H), 1.02-1.75 (m, 23H), 0.67-1.02 (M, 6H), 0.58 (s, 3H).

### Example 19: Synthesis of Compound 203

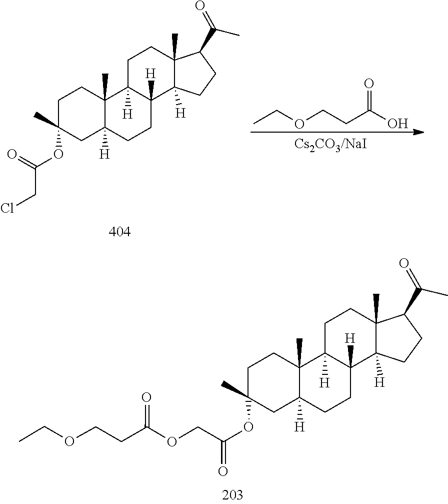

In a similar manner to the procedure outlined in Example 17 (not including the step for forming an HCl salt), substituting 4-morpholineacetic acid with 3-ethoxypropionic acid, Compound 203 was obtained. HRMS (ESI+) calculated for C₂₉H₄₆NaNO₆ m/z [M+H]⁺: 513.3187, observed 513.3198; ¹H NMR δ: 4.46-4.64 (m, 2H), 3.66-3.81 (m, 2H), 3.43-3.59 (m, 2H), 2.63-2.77 (m, 2H), 2.45-2.57 (m, 1H), 2.05-2.29 (m, 5H), 1.93-2.04 (m, 1H), 1.81-1.92 (m, 1H), 1.05-1.73 (m, 22H), 0.71-1.00 (m, 5H), 0.51-0.66 (s, 3H).

### Example 20: Synthesis of Compound 204

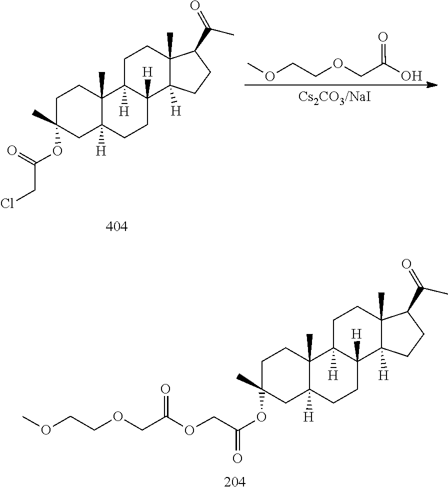

In a similar manner to the procedure outlined in Example 17 (not including the step for forming an HCl salt), substituting 4-morpholineacetic acid with 2-(2-methoxyethoxy)acetic acid, Compound 204 was obtained. HRMS (ESI+) calculated for C₂₉H₄₆NaNO₇ m/z [M+H]⁺: 529.3136, observed 529.3137; ¹H NMR δ: 4.54-4.72 (m, 2H), 4.27 (s, 2H), 3.71-3.83 (m, 2H), 3.56-3.69 (m, 2H), 3.33-3.47 (m, 3H), 2.44-2.59 (m, 1H), 2.07-2.28 (m, 5H), 1.82-2.05 (m, 2H), 1.07-1.76 (m, 19H), 0.70-1.01 (m, 5H), 0.58 (s, 3H).

### Example 21: Synthesis of Compound 205

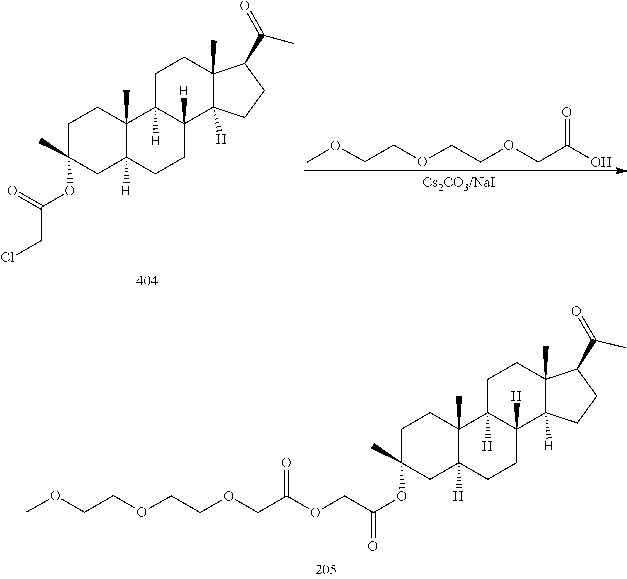

In a similar manner to the procedure outlined in Example 17 (not including the step for forming an HCl salt), substituting 4-morpholineacetic acid with 2-[2-(2-methoxyethoxy)ethoxy]acetic acid, Compound 205 was obtained. HRMS (ESI+) calculated for C₃₁H₅₀NaO₈ m/z [M+H]⁺: 573.3398, observed 573.3399; ¹H NMR δ: 4.51-4.69 (m, 2H), 4.25 (s, 2H), 3.50-3.86 (m, 8H), 3.36 (s, 3H), 2.46-2.55 (m, 1H), 2.06-2.27 (m, 6H), 1.97 (d, J=11.72 Hz, 1H), 1.86 (d, J=11.14 Hz, 2H), 1.09-1.73 (m, 18H), 0.71-1.01 (m, 4H), 0.57 (s, 3H).

### Example 22: Synthesis of Compound 206

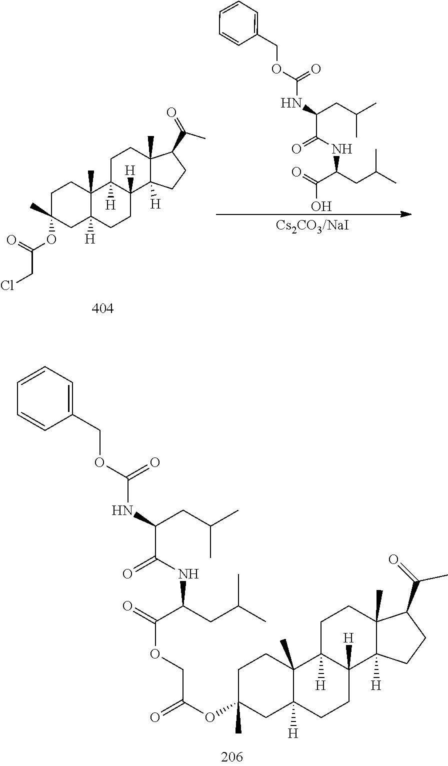

In a similar manner to the procedure outlined in Example 17 (not including the step for forming an HCl salt), substituting 4-morpholineacetic acid with N-[(phenylmethoxy)carbonyl]-L-leucyl-L-leucine, Compound 206 was obtained. HRMS (ESI+) calculated for C₄₄H₆₇N₂O₈ m/z [M+H]⁺: 751.489194, observed 751.489134; ¹H NMR δ: 7.22-7.42 (m, 5H), 6.52 (d, J=7.62 Hz, 1H), 5.35 (d, J=8.21 Hz, 1H), 5.08 (s, 2H), 4.55-4.73 (m, 2H), 4.20-4.24 (m, 1H), 2.41-2.56 (m, 1H), 1.83-2.27 (m, 8H), 1.08-1.78 (m, 24H), 0.83-1.00 (m, 13H), 0.67-0.80 (m, 4H), 0.57 (s, 3H).

### Example 23: Synthesis of Compound 207

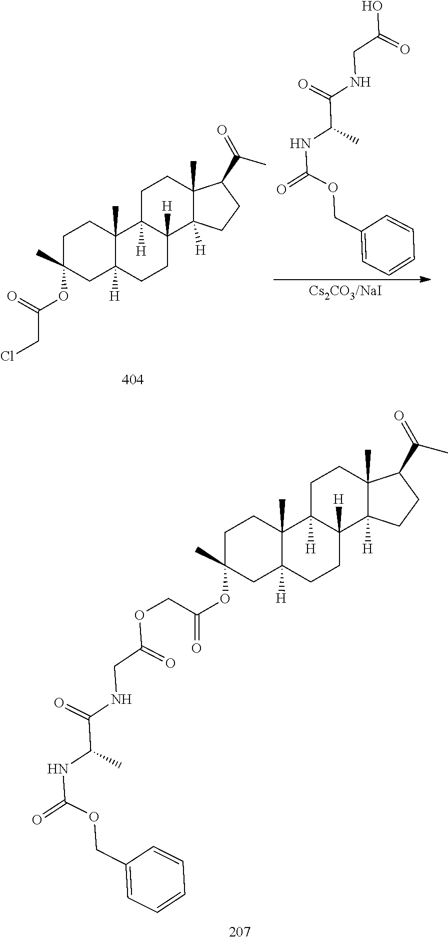

In a similar manner to the procedure outlined in Example 17 (not including the step for forming an HCl salt), substituting 4-morpholineacetic acid with CBz-L-Ala-Gly-OH, Compound 207 was obtained. HRMS (ESI+) calculated for C₃₇H₅₃N₂O₈ m/z [M+H]⁺: 653.379643, observed 653.378352; ¹H NMR δ: 7.28-7.45 (m, 5H), 6.74 (br. s. 1H), 5.44 (d, J=6.45 Hz, 1H), 5.03-5.23 (m, 2H), 4.51-4.68 (m, 2H), 4.24-4.41 (m, 1H), 4.10-4.24 (m, 2H), 2.46-2.59 (m, 1H), 2.07-2.30 (m, 5H), 1.85-2.04 (m, 2H), 1.07-1.77 (m, 20H), 0.83-1.02 (m, 1H), 0.68-1.04 (m, 7H), 0.59 (s, 3H).

### Example 24: Synthesis of Compound 208

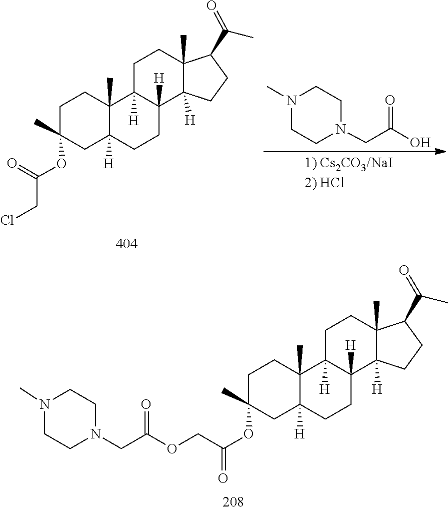

In a similar manner to the procedure outlined in Example 17, substituting 4-morpholineacetic acid with 4-methyl-1-piperazineacetic acid, Compound 208 was obtained as a bis-HCl salt. HRMS (ESI+) calculated for C₃₁H₅₁N₂O₅ m/z [M+H]⁺: 531.379249, observed 531.378222; ¹H NMR (MeOD) δ: 4.88-5.00 (m, 4H), 4.73-4.85 (m, 2H), 4.29-4.43 (m, 2H), 3.02 (s, 3H), 2.63 (t, J=8.79 Hz, 1H), 1.98-2.27 (m, 7H), 1.86-1.95 (m, 1H), 1.13-1.78 (m, 23H), 0.92-1.08 (m, 2H), 0.73-0.86 (m, 4H), 0.60 (s, 3H).

### Example 25: Synthesis of Compound 201

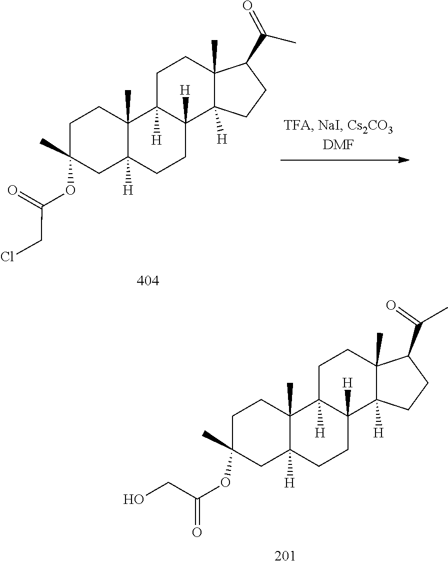

To a suspension of compound 404 (72.1 mg, 0.18 mmol), trifluoroacetic acid (100 mg, 0.88 mmol) and sodium iodide (30 mg, 0.20 mmol) was added in 3 mL of anhydrous DMF to produce a clear orange solution. This solution was allowed to stir at ambient temperature for 15 minutes. After 15 mins, Cs₂CO₃ (278 mg, 0.85 mmol) was added and the resulting mixture was heated to 70° C. After stirring for 18 h, the reaction was cooled, diluted with 25 mL of EtOAc, and then quenched with 15 mL of water. After separation of the layers, the EtOAc layer was washed with 2×15 ml of saturated NaHCO₃ solution, dried with Na₂SO₄, filtered and concentrated to provide a clear orange oil. The crude mixture was purified by column chromatography. The oil was dissolved in DCM and stripped on to silica gel. The silica gel was dried under high vacuum for 30 minutes, then loaded onto a 4-gram cartridge and eluted with EtOAc in hexanes running 0-10% EtOAc over 10 volumes, hold at 10% for 10 volumes, then ramping to 50%, followed by a final ramp to 100% EtOAc. Compound 201 (73.3 mg) was obtained as a crystalline solid. HRMS (ESI+): calculated for C₂₄H₃₈NaNO₄ m/z [M+H]⁺: 413.2662, observed 413.2662; ¹H NMR 300 MHz CDCl3 δ: 4.04-4.11 (m, 2H), 2.52 (t, J=9.08 Hz, 1H), 2.38-2.45 (m, 1H), 2.08-2.27 (m, 5H), 1.89-2.05 (m, 2H), 1.05-1.73 (m, 20H), 0.87-1.01 (m, 1H), 0.67-0.80 (m, 4H), 0.59 (s, 3H).

### Example 26: Synthesis of Compound 500

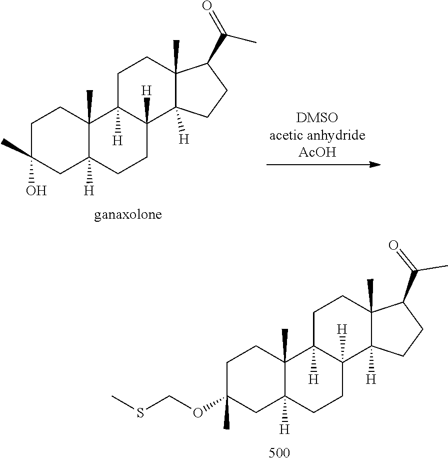

Ganaxolone (2.53 g, 7.61 mmol) was added to a solution of 75 mL of DMSO and acetic anhydride (26 mL, 275.05 mmol) to produce a thin slurry. To this slurry was added acetic acid (0.5 mL, 8.73 mmol) and the reaction was stirred at room temperature overnight resulting in a clear solution. The reaction was diluted with 300 mL of EtOAc, followed by 75 mL of H₂O. The mixture was agitated and allowed to settle. The lower aqueous layer was dropped and the process was repeated a total of 4 times. The washed organic was dried with sodium sulfate, filtered and concentrated to provide a clear faint yellow oil, which was dried under vacuum. The crude reaction mixture was purified via column chromatography. The oil was loaded onto an 80-gram cartridge eluting with 0.5% to 3% EtOAc/hexanes. The desired fractions were combined and concentrated. Compound 500 was obtained as a solid after drying under vacuum (2.31 g). HRMS (ESI+): calculated for C24H4102S m/z [M+H]⁺: 393.2822, observed 393.2830; ¹H NMR 300 MHz CDCl3 δ: 4.36-4.50 (m, 2H), 2.46-2.57 (m, 1H), 2.06-2.23 (m, 7H), 1.94-2.06 (m, 2H), 1.10-1.79 (m, 22H), 0.83-1.01 (m, 1H), 0.70-0.81 (m, 4H), 0.59 (s, 3H).

### Example 27: Synthesis of Compound 503

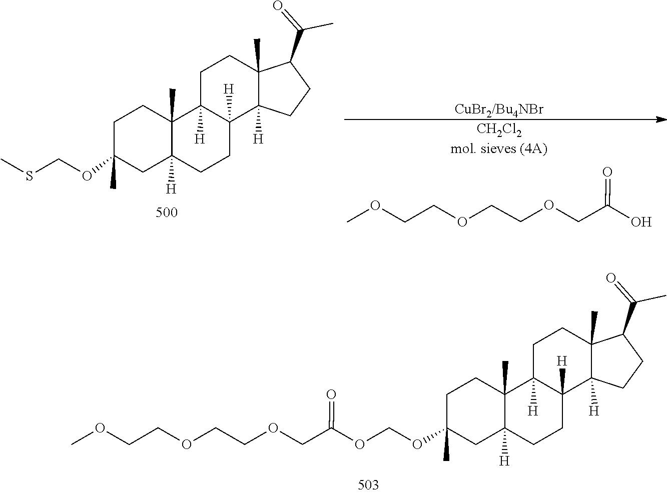

A molecular sieves (0.25 g) were added to a solution of compound 500 (107 mg, 0.27 mmol), 2-[2-(2-methoxyethoxy)ethoxy]acetic acid (60.4 mg, 0.34 mmol) and 3 mL of dichloromethane to produce a thick white slurry. The reaction was cooled to 0° C. in an ice bath and Bu₄NBr (0.1 mg, 0.31 mmol) was added followed by CuBr₂ (68.5 mg, 0.31 mmol). Upon addition of the CuBr₂, the reaction turned a deep purple color. The reaction was warmed to ambient temperature. After stirring for 18 h, the reaction was diluted with 25 mL of EtOAc, then filter to remove the molecular sieves. The sieves were washed with 15 ml of EtOAc. The combined organic phase was then washed with 2×15 ml of saturated NaHCO₃ solution, followed by 15 mL of sat. NaCl solution. The washed organic phase was dried with Na₂SO₄, filtered and concentrated to provide a clear orange oil. The crude mixture was purified by column chromatography. The oil was dissolved in CH₂Cl₂ and stripped on to silica gel. The silica gel was dried under high vacuum for 30 minutes, then loaded onto a 4-gram cartridge and eluted with EtOAc in hexanes: 15% EtOAc, hold for 10 volumes then ramp to 100 over 5 volumes then hold for 10 volumes. Compound 503 (57.3 mg) was obtained as an oil. HRMS (ESI+): calculated for C₃₀H₅₀NaO₇ m/z [M+H]⁺: 545.3449, observed 545.3448; ¹H NMR 300 MHz CDCl3 δ: 5.33-5.44 (m, 2H), 4.10 (s, 2H), 3.59-3.77 (m, 6H), 3.49-3.57 (m, 2H), 3.34 (s, 3H), 2.44-2.55 (m, 1H), 2.05-2.18 (m, 4H), 1.91-2.01 (m, 1H), 1.09-1.77 (m, 21H), 0.97-1.08 (m, 1H), 0.70-0.96 (m, 4H), 0.55 (s, 3H).

### Example 28: Synthesis of Compound 502

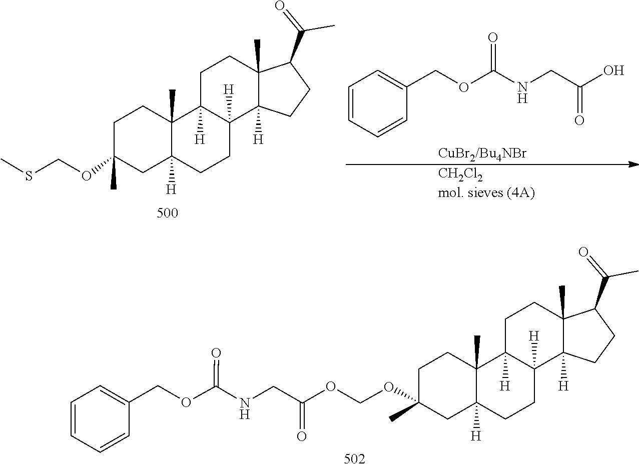

In a similar manner to the procedure outlined in Example 27, substituting 2-[2-(2-methoxyethoxy)ethoxy]acetic acid with N-Cbz-glycine, Compound 502 was obtained. HRMS (ESI+) calculated for C₃₃H₄₇NNaO₆ m/z [M+H]⁺: 576.3296, observed 576.3287; ¹H NMR δ: 7.27-7.45 (m, 5H), 5.37-5.50 (M, 2H), 5.24-5.32 (m, 1H), 5.10-5.18 (m, 2H), 3.95-4.02 (m, 2H), 2.46-2.57 (m, 1H), 2.07-2.20 (m, 4H), 1.92-2.05 (m, 1H), 1.10-1.82 (m, 21H), 0.74-1.04 (m, 5H), 0.55-0.69 (m, 3H).

### Example 29: Synthesis of Compound 209

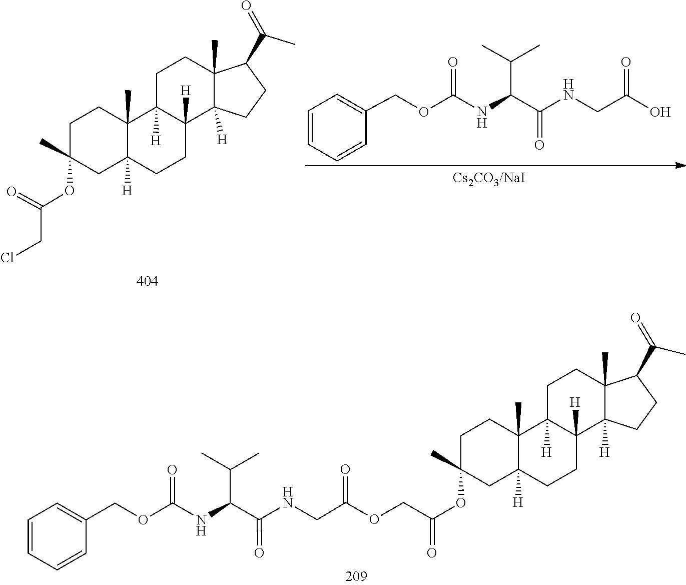

In a similar manner to the procedure outlined in Example 17 (not including the step for forming an HCl salt), substituting 4-morpholineacetic acid with Cbz-L-valyl-glycine, Compound 209 was obtained. HRMS (ESI+) calculated for C₃₉H₅₇N₂O₈ m/z [M+H]⁺: 681.410943, observed 681.411352; ¹H NMR δ: 7.30-7.41 (m, 5H), 6.81 (d, J=4.69 Hz, 1H), 5.53 (d, J=9.38 Hz, 1H), 5.06-5.14 (m, 2H), 4.55-4.63 (m, 2H), 4.05-4.20 (m, 3H), 2.46-2.58 (m, 1H), 2.07-2.27 (m, 5H), 1.93-2.04 (m, 2H), 1.82-1.92 (m, 1H), 1.43-1.75 (m, 1OH), 1.08-1.43 (m, 1H), 0.89-1.07 (m, 6H), 0.71-0.84 (m, 3H), 0.57 (s, 3H).

### Example 30: Synthesis of Compound 210

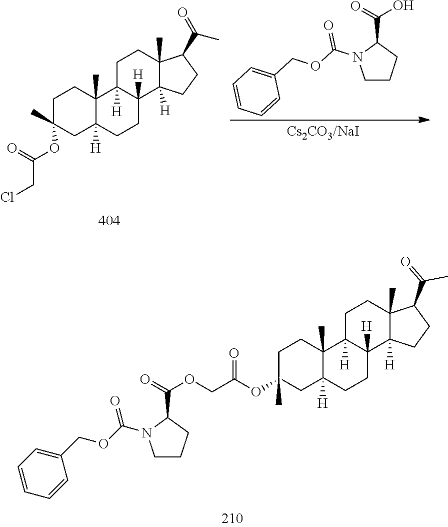

In a similar manner to the procedure outlined in Example 17 (not including the step for forming an HCl salt), substituting 4-morpholineacetic acid with Cbz-D-proline, Compound 210 was obtained. HRMS (ESI+) calculated for C₃₇H₅₂NO₇ m/z [M+H]⁺: 644.355774, observed 644.355320; ¹H NMR δ: 7.26-7.44 (m, 5H), 5.06-5.22 (m, 2H), 4.33-4.55 (m, 2H), 3.59-3.71 (m, 1H), 3.43-3.58 (m, 1H), 2.53 (t, J=8.79 Hz, 1H), 2.16-2.30 (m, 3H), 2.11 (s, 3H), 1.80-2.04 (m, 4H), 1.10-1.74 (m, 21H), 0.83-1.02 (m, 2H), 0.76 (s 3H), 0.60 (s, 3H).

### Example 31: Synthesis of Compound 211

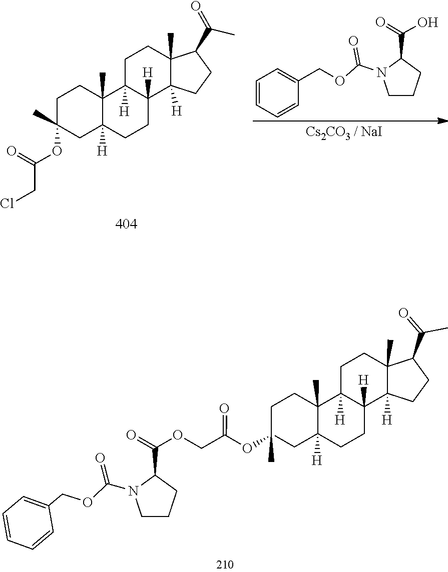

In a similar manner to the procedure outlined in Example 17, substituting 4-morpholineacetic acid with (S)-2-morpholin-4-yl-propionic acid hydrochloride, Compound 211 was obtained as a hydrochloride salt. HRMS (ESI+) calculated for C₃₁H₅₀NO₆ m/z [M+H]⁺: 532.363265, observed 532.362510; ¹H NMR δ: 4.08-4.30 (m, 2H), 3.74-4.07 (m, 2H), 3.39-3.74 (m, 4H), 2.80-3.28 (m, 5H), 1.95-2.13 (m, 1H), 1.47-1.83 (m, 7H), 0.59-1.47 (m, 21H), 0.43 (br s, 1H), 0.19-0.36 (m, 4H), 0.04-0.19 (m, 3H).

### Example 32: Synthesis of Compound 212

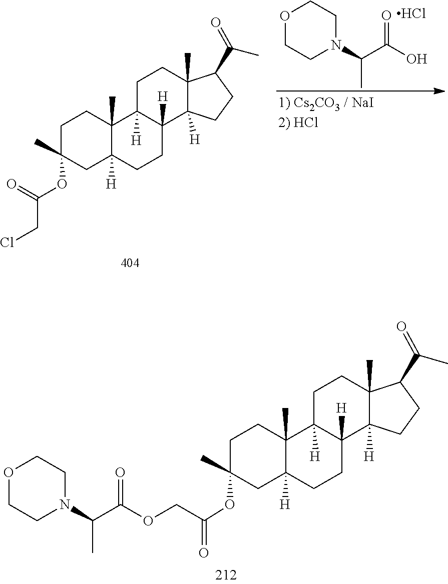

In a similar manner to the procedure outlined in Example 17, substituting 4-morpholineacetic acid with (R)-2-morpholin-4-yl-propionic acid hydrochloride, Compound 212 was obtained as a hydrochloride salt. HRMS (ESI+) calculated for C₃₁H₅₀NO₆ m/z [M+H]⁺: 532.363265, observed 532.363688; ¹H NMR δ: 4.57-4.76 (m, 2H), 4.24-4.52 (m, 2H), 3.92-4.17 (M, 3H), 3.29-3.70 (m, 4H), 2.52 (t, J=8.79 Hz, 1H), 2.08-2.29 (m, 5H), 2.01 (d, J=11.14 Hz, 1H), 1.85 (d, J=4.69 Hz, 3H), 1.53-1.76 (m, 9H), 1.49 (s, 3H), 1.04-1.47 (m, 11H), 0.87-1.03 (m, 1H), 0.69-0.82 (m, 3H), 0.60 (s, 3H).

### Example 33: Synthesis of Compound 213

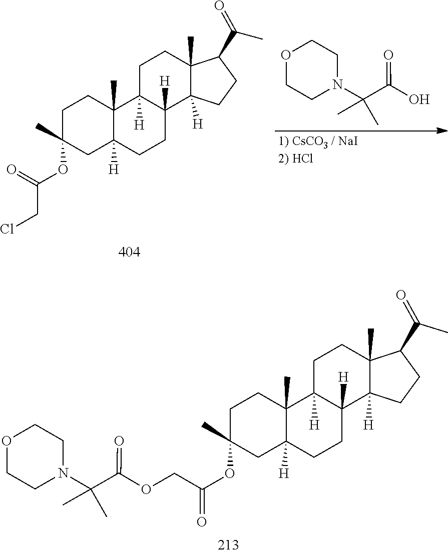

In a similar manner to the procedure outlined in Example 17, substituting 4-morpholineacetic acid with α,α-dimethyl-4-morpholineacetic acid hydrochloride, Compound 213 was obtained as a hydrochloride salt. HRMS (ESI+) calculated for C₃₂H₅₂NO₆ m/z [M+H]⁺: 546.378915, observed 546.378119; ¹H NMR δ: 4.59-4.75 (m, 2H), 4.42-4.58 (m, 2H), 3.98 (d, J=12.31 Hz, 2H), 3.54 (br. s 4H), 2.51 (t, J=9.08 Hz, 1H), 2.07-2.28 (m, 6H), 1.80-2.06 (m, 9H), 1.04-1.75 (m, 24H), 0.85-1.01 (m, 1H), 0.68-0.81 (m, 4H), 0.60 (s, 3H).

### Example 34: Synthesis of Compound 111

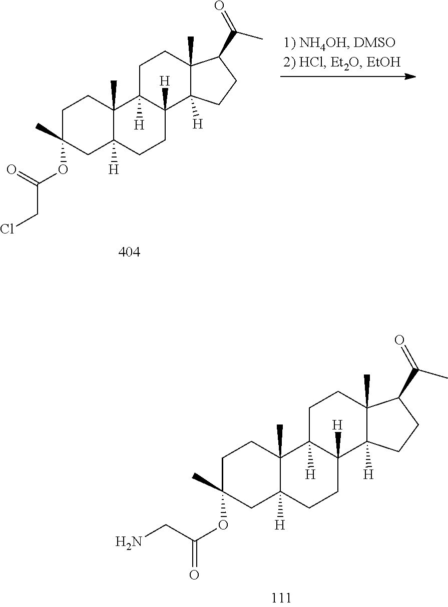

Compound 404 (114 mg 0.28 mmol) was dissolved in 5 mL of DMSO to produce a clear nearly colorless solution after 10 minutes. To this solution was added ammonium hydroxide 28-30 wt % (0.25 mL, 2.07 mmol) causing an immediate color change to a red-orange. The reaction was allowed to stir at ambient temperature. After stirring for 18 h, the reaction was worked up as follows: dilute with 25 mL of EtOAc, then quench with 15 mL of water. Transfer to a separatory funnel and drop the lower aqueous. Extract with 2×15 ml of H₂O, dropping the lower aqueous each time. The washed organic layers were then dried with Na₂SO₄ for 30 minutes, filtered and concentrated to provide a clear orange oil upon drying. The sample was dissolved in 2 mL of absolute EtOH to produce a clear light brown solution. To this was charged 2N HCl in Et₂O (0.2 mL, 0.4 mmol), the solution darkened, but no solids were observed. After stirring for 30 minutes, the reaction was placed on the rotary evaporator and the solvent removed. This resulted in the formation of a foam that was allowed to dry under high vacuum, to provide Compound 111 (91.2 mg) as a hydrochloride salt. HRMS (ESI+): calculated for C₂₄H₄₁ClNO₃ m/z [M+H]⁺: 390.300271, observed 390.300526; ¹H NMR (MeOD) δ: 3.98 (d, J=2.34 Hz, 1H), 3.25-3.42 (m, 2H overlaps with solvent), 2.61 (t, J=8.50 Hz, 1H), 1.89-2.28 (m, 6H), 1.15-1.79 (m, 24H), 0.75-1.06 (m, 4H), 0.60 (s, 3H).

### Example 35: Synthesis of Compound 112

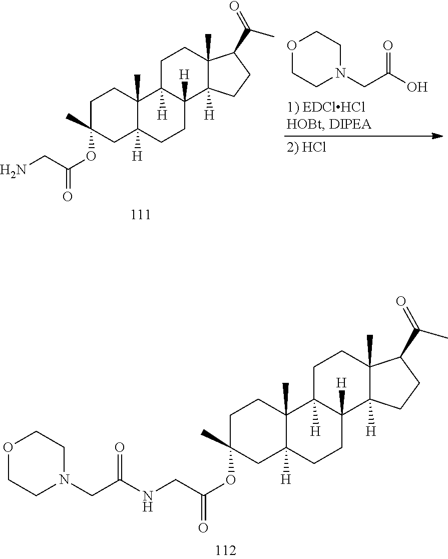

DCM (4 mL) was added to compound 111 (from Example 34, 31.6 mg, 0.07 mmol) and 4-morpholinylacetic acid (13.2 mg, 0.09 mmol). The mixture was cooled to 0° C. in an ice water bath and N-(3-dimethylaminopropyl)—N′-ethylcarbodiimide hydrochloride (19 mg, 0.10 mmol), and hydroxybenzotriazole (14.5 mg, 0.11 mmol) were added to produce a slurry that clarified upon the addition of diisopropylethylamine (40 μL, 0.23 mmol). The resulting orange solution was allowed to warm to ambient temperature overnight. The reaction was diluted with 10 mL of DCM and quenched with 10 mL of H₂O. The mixture was transferred to a separatory funnel and the phases were separated. The organic was then washed with 10 mL of saturated NaHCO₃, then 10 mL of NaCl. The washed organic was dried with Na₂SO₄, filtered and concentrated to provide the crude product. The crude product was purified by column chromatography using an ISCO™ chromatography system to provide compound 112 (free base) as a solid (17.1 mg). HRMS (ESI+): calculated for C₃₀H₄₉N₂O₅ m/z [M+H]⁺: 517.3636, observed 517.3635; ¹H NMR δ: 7.57 (br. s., 1H), 3.93-4.16 (m, 3H), 3.66-3.83 (m, 5H), 3.06 (s, 2H), 2.45-2.65 (m, 6H), 1.93-2.30 (m, 9H), 1.85 (d, J=12.89 Hz, 1H), 1.04-1.75 (m, 27H), 0.84-1.02 (m, 2H), 0.68-0.81 (m, 5H), 0.59 (s, 3H).

The resulting free base was treated with 2.0 M HCl/diethyl ether (2 mL), and solvent was removed in vacuo to provide Compound 112 as a solid hydrochloride salt (19.8 mg). HRMS (ESI+): calculated for C₃₀H₄₉N₂O₅ m/z [M+H]⁺: 517.3636, observed 517.3635; ¹H NMR δ: 9.07 (br. s., 1H), 4.11 (br. s., 4H), 3.96 (br. s., 4H), 3.74 (br. s., 2H), 3.28 (br. s., 2H), 2.60-2.49 (m, 1H), 2.30-2.07 (m, 6H), 2.06-1.95 (mm 2H), 1.83 (d, J=13.5 hz, 2H), 1.76-7.56 (m, 5H), 1.54-1.08 (m, 21H), 1.00 (br. s., 1H), 0.85-0.71 (m, 5H), 0.60 (s, 3H).

### Example 36: Synthesis of Compound 114

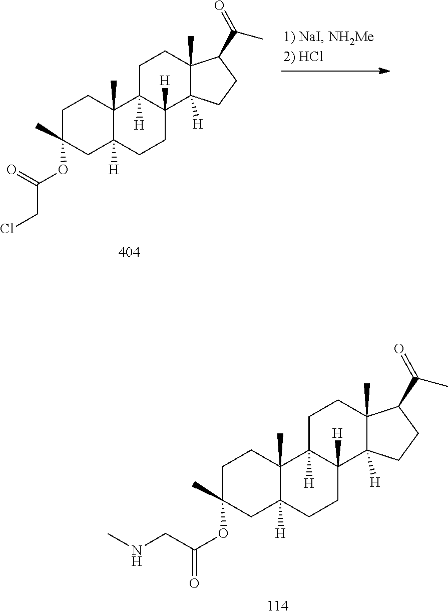

Compound 404 (125 mg, 0.31 mmol) and sodium iodide (46.7 mg, 0.31 mmol) were dissolved in 3 mL of anhydrous DMF to produce a clear orange solution. This solution was allowed to stir at ambient temperature for 15 minutes and then a 2M solution of methylamine in tetrahydrofuran (THF) (150 μL, 0.30 mmol) was added followed by Cs₂CO₃ (127.4 mg, 0.39 mmol) and the resulting mixture was heated to 70° C. After stirring for 2 h, the reaction was cooled, diluted with 25 mL of EtOAc, and then quenched with 15 mL of water. After separation of the layers the EtOAc layer was washed with 2×15 ml of saturated NaHCO₃ solution. The washed organic phase was dried with Na₂SO₄, filtered and concentrated to provide a clear orange oil. The crude product was purified by column chromatography using an ISCO™ chromatography system to provide compound 114 (free base; 36.1 mg) as a solid. HRMS (ESI+): calculated for C₂₅H₄₂NO₃ m/z [M+H]⁺: 404.3159, observed 404.3159; ¹H NMR δ: 3.36-3.54 (m, 2H), 2.42-2.59 (m, 4H), 1.81-2.30 (m, 8H), 10.03-1.73 (m, 23H), 0.82-1.02 (m, 2H), 0.65-0.82 (m, 5H), 0.49-0.63 (m, 3H).

Diethyl ether (Et₂O; 2 mL) was added to the free base 114 (33.6 mg, 0.08 mmol) to produce a clear solution. The solution was allowed to stir for 5 minutes. 2M HCl in Et₂O (50 ul 0.100 mmol) was added to the reaction flask and a precipitate formed. The reaction was stirred for an additional 10 minutes. The precipitate was isolated by filtration and washed with 5 mL of Et₂O and dried under vacuum to obtain Compound 114 as a hydrochloride salt (31.4 mg). HRMS (ESI+): calculated for C25H42NO3 m/z [M+H]⁺: 404.3159, observed 404.3159; ¹H NMR δ: 3.78 (br. s., 2H), 2.84 (br. s., 3H), 2.53 (t, J=8.50 Hz, 1H), 2.08-2.35 (m, 5H), 2.00 (d, J=11.14 Hz, 1H), 1.89 (d, J=8.21 Hz, 1H), 1.07-1.78 (m, 23H), 0.97 (d, J=8.21 Hz, 1H), 0.72-0.85 (m, 4H), 0.60 (s, 3H).

### Example 37: Synthesis of Compound 115

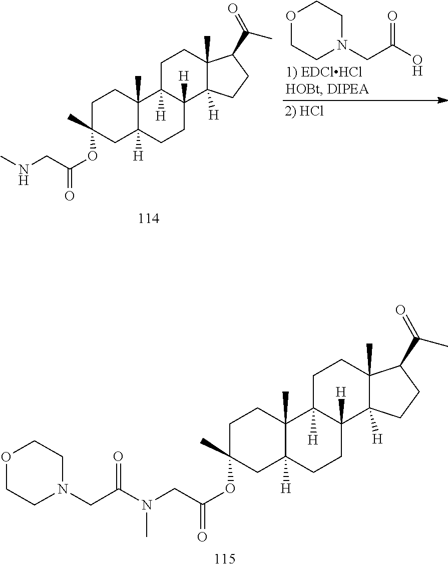

In a similar manner to the procedure outlined in Example 35, substituting Compound 404 with Compound 114, compound 115 was obtained as a hydrochloride salt (18.7 mg). HRMS (ESI+): calculated for C₃₁H₅₁N₂O₅ m/z [M+H]⁺: 531.3792, observed 531.3782; ¹H NMR δ: 4.25 (s, 1H), 4.17-3.95 (m, 2H), 3.66-3.86 (m, 7H), 3.25 (s, 2H), 3.22-3.12 (m, 4H), 2.98 (s, 2H), 2.58-2.52 (m, 8H), 2.40-2.09 (m, 11H), 2.07-1.80 (m, 4H), 1.79-1.08 (m, 33H), 1.03-0.75 (m, 7H), 0.74-0.57 (m, 4H).

### Example 38: Synthesis of Compound 218

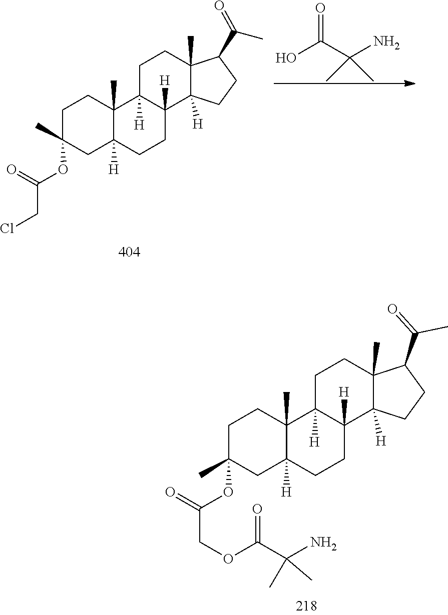

In a similar manner to the procedure outlined in Example 17, substituting 4-morpholineacetic acid with 2-aminoisobutyric acid, Compound 218 (101.6 mg) was obtained as a solid. HRMS (ESI+): calculated for C28H46NO5 m/z [M+H]⁺: 476.3370, observed 476.3370; ¹H NMR δ: 4.63-4.45 (m, 2H), 2.56-2.43 (m, 1H), 2.30-2.03 (m, 6H), 2.01-1.92 (m, 1H), 1.89-1.73 (m, 4H), 1.72-1.05 (m, 29H), 1.00-0.82 (m, 1H), 0.81-0.66 (m, 4H), 0.62-0.51 (m, 3H).

### Example 39: Preparation of Compound 125

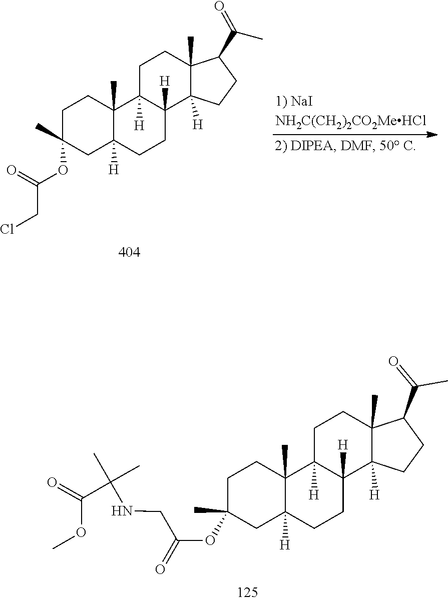

Anhydrous DMF (3 mL) was added compound 404 (150 mg, 0.37 mmol) and sodium iodide (96 mg, 0.64 mmol) to produce a clear orange solution. This solution was allowed to stir at ambient temperature for 15 minutes and methyl-2-aminoisobutyrate·HCl (302 mg, 1.96 mmol) was added followed by diisopropylethylamine (850 μL, 0.49 mmol) and the resulting mixture was heated to 50° C. After 22 h, the reaction was cooled. The reaction was diluted with 25 mL of EtOAc, then quenched with 15 mL of water. After separation of layers the EtOAc layer was washed with 2×15 ml of 1N HCl, and then 15 mL of brine. The washed organic was dried with Na₂SO₄ for 30 minutes, filtered and concentrated to provide a clear light brown oil. The crude product was purified by column chromatography using an ISCO™ chromatography system to provide Compound 125 (85.1 mg) as an oil. HRMS (ESI+): calculated for C₂₉H₄₈NO₅ m/z [M+H]⁺: 490.3527, observed 490.3527; ¹H NMR δ: 3.71 (s, 3H), 3.30 (s, 2H), 2.52 (t, J=8.8 Hz, 1H), 2.30-2.08 (m, 6H), 2.05-1.87 (m, 2H), 1.74-1.38 (m, 20H), 1.38-1.33 (m, 8H), 0.92 (dq, J=5.3, 11.9 Hz, 1H), 0.81-0.68 (m, 4H), 0.60 (s, 3H).

### Example 40: Synthesis of Compound 126

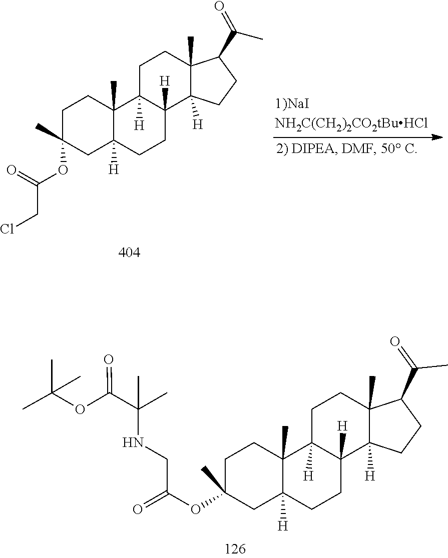

In a similar manner to the procedure outlined in Example 39, substituting methyl-2-aminoisobutyrate·HCl with tert-butyl 2-amino-2-methylpropanoate·HCl (302 mg, 1.96 mmol), compound 126 (188 mg) was obtained as a solid. HRMS (ESI+): calculated for C₃₂H₅₄NO₅ m/z [M+H]⁺: 532.3997, observed 532.3999; ¹H NMR δ: 8.03 (s, 1H), 3.79-3.56 (m, 2H), 2.96 (s, 3H), 2.89 (s, 3H), 2.52 (t, J=8.8 Hz, 1H), 2.31-2.08 (m, 6H), 2.04-1.88 (m, 2H), 1.78-1.11 (m, 27H), 1.06-0.89 (m, 1H), 0.85-0.73 (m, 4H), 0.60 (s, 3H).

### Example 41: Synthesis of Compound 305

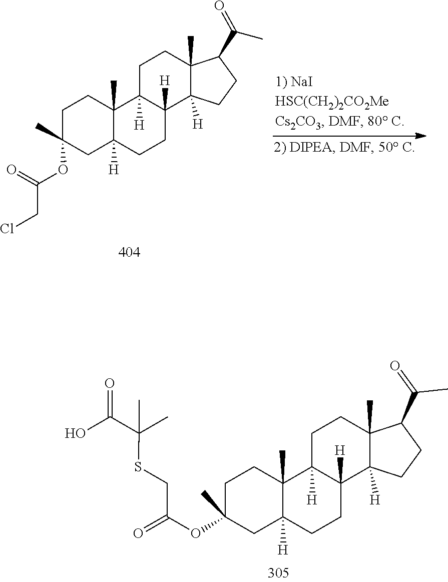

In a similar manner to the procedure outlined in Example 39, substituting methyl-2-aminoisobutyrate·HCl with 2-mercapto-2-methylpropanoic acid methyl ester, a methyl ester compound (39.1 mg) was obtained as an oil that was used directly in the next step without further purification. HRMS (ESI+): calculated for C₂₉H₄₇O₅S m/z [M+H]⁺: 507.313872, observed 507.313659; ¹H NMR δ: 3.77-3.69 (m, 3H), 3.37 (s, 2H), 2.58-2.47 (m, 1H), 2.23-2.08 (m, 6H), 2.04-1.88 (m, 2H), 1.74-1.43 (m, 21H), 1.42-1.32 (m, 4H), 1.31-1.11 (m, 8H), 0.92 (dq, J=5.3, 11.9 Hz, 1H), 0.82-0.71 (m, 4H), 0.59 (s, 3H).

Aqueous NaOH (1.0 M, 1 mL) was added to the ester at room temperature (62 mg, 0.13 mmol) in methanol (5 mL). After 18 hr the reaction was diluted with 25 mL of EtOAc, and then quenched with 15 mL of H₂O. The layers were separated and the aqueous layer was acidified with 2.0 mL of 2N HCl to a pH of 1. The aqueous layer was extracted 2×15 mL of DCM. The DCM phase was dried using Na₂SO₄, filtered and concentrated to provide Compound 305 as an oil (10 mg). ¹H NMR δ: 3.42 (s, 2H), 2.51 (t, J=9.08 Hz, 1H), 2.26-2.06 (m, 6H), 2.06-1.85 (m, 3H), 1.72-1.58 (m, 4H), 1.54 (s, 6H), 1.51-1.45 (m, 5H), 1.42-1.31 (m, 5H), 1.30-1.08 (m, 1OH), 1.00-0.81 (m, 2H), 0.80-0.70 (m, 4H), 0.58 (s, 3H).

### Example 42: Synthesis of Compound 307

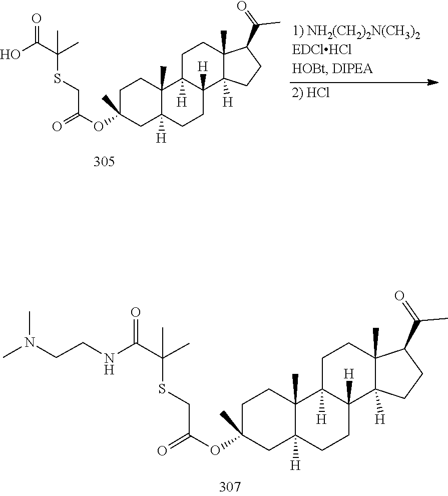

N,N-Dimethyl-1,2-ethylenediamine (38.9 mg, 0.44 mmol) and Compound 305 (45 mg, 0.091 mmol) were dissolved in 4 ml of DCM. The mixture was cooled to −0° C. in an ice bath and EDCI·HCl (22 mg, 0.11 mmol), HOBt (16 mg, 0.12 mmol) and DIPEA (40 l, 0.23 mmol) were added. The resulting pale-yellow solution was allowed to slowly warm to ambient temperature overnight. After 21 h the reaction was diluted with 25 mL of EtOAc, then quenched with 15 ml of H₂O. The mixture was transferred to a separatory funnel and the lower aqueous was discarded. The organic was washed with 15 mL of saturated NaHCO₃ solution, then 15 ml of saturated brine. The organic layer was then dried with Na₂SO₄, filtered and concentrated. The crude product was purified by column chromatography using an ISCO™ chromatography system to provide Compound 307 as an oil (free base; 34.3 mg). The free base was treated with 2.0 M HCl/diethyl ether (2 mL), and solvent was removed in vacuo to provide Compound 307 as a solid hydrochloride salt (38.3 mg). HRMS (ESI+): calculated for C₃₂H₅₅N₂O₄S m/z [M+H]⁺: 563.3863, observed 563.3877; ¹H NMR δ: 8.10 (br. s., 1H), 3.76-3.53 (m, 3H), 3.38 (s, 2H), 3.21 (t, J=5.0 Hz, 2H), 2.88 (s, 6H), 2.53 (t J=9.1 Hz, 1H), 2.25-2.06 (m, 6H), 1.99 (d, J=12.3 Hz, 1H), 1.87 (d, J=14.1 Hz, 1H), 1.73-1.09 (m, 24H), 1.02-0.70 (m, 5H), 0.59 (s, 3H).

### Example 43: Synthesis of Compound 124

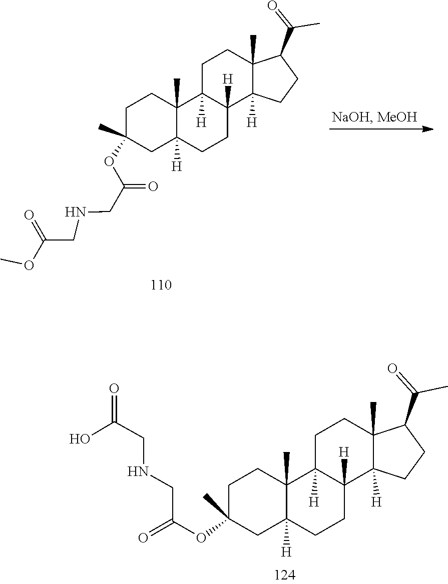

Compound 110 (from Example 12; 62 mg, 0.13 mmol) was dissolved in 5 mL of MeOH. 1 mL of 1M aqueous NaOH was added and the resulting solution was allowed to stir at ambient temperature. After 21 h the reaction was diluted with 25 mL of EtOAc, then quenched with 15 ml of H₂O. The mixture was transferred to a separatory funnel and the upper organic was discarded. The aqueous layer was then dried acidified with 2.0 mL of 2N HCl to a pH of 1. The aqueous layer was extracted 2×15 mL of DCM. The organic was dried using Na₂SO₄, filtered and concentrated to provide Compound 124 as an oil (58.9 mg). HRMS (ESI+): calculated for C₂₆H₄₂NO₅ m/z [M+H]⁺: 448.3027, observed 448.3051; ¹H NMR δ: 4.01-3.88 (m, 2H), 3.68 (s, 2H), 2.62 (t, J=8.8 Hz, 1H), 2.30-1.86 (m, 7H), 1.78-1.10 (m, 19H), 1.07-0.69 (m, 7H), 0.60 (s, 3H).

### Example 44: Synthesis of Compound 304

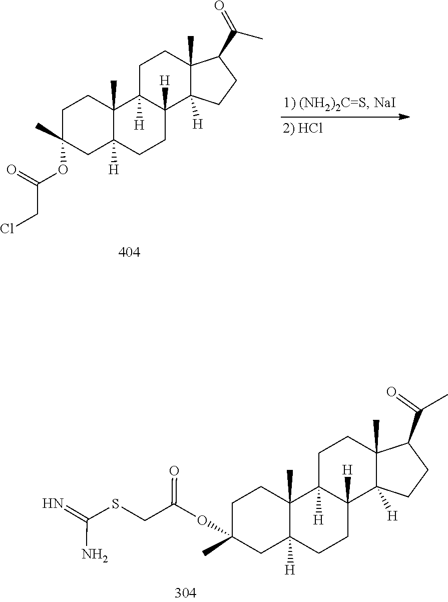

Compound 404 (74 mg, 0.18 mmol) and sodium iodide (49 mg, 0.33 mmol) were suspended in 5 mL of 1,4 dioxane, thiourea (14.5 mg, 0.19 mmol) was added and the mixture was then heated to 85° C. After stirring for 18 h, the reaction was cooled, diluted with 25 mL of EtOAc, then quenched with 15 mL of water. The EtOAc layer was washed with 1×15 ml of H₂O, then 15 mL of brine and dried with Na₂SO₄ to then in vacuo provide a yellow solid. The solid was then taken up in 10 mL of 1,4 dioxane to provide a clear yellow solution. To this was added 4N HCl in 1,4 dioxane (50 ul, 0.200 mmol). The solution was allowed to stir for 15 minutes, then was frozen in dry ice. The yellow solid was placed on the lyophilizer and freeze dried to provide compound 304 as a solid hydrochloride salt (77.8 mg). HRMS (ESI+): calculated for C25H41C1N2O3S m/z [M+H]⁺: 449.2832, observed 449.2827; ¹H NMR δ: 9.63-8.79 (bs. s., 3H), 4.03-3.84 (s, 1H), 3.77-3.58 (m, 3H), 2.51 (q, J=8.8 Hz, 1H), 2.26-2.02 (m, 6H), 1.51-1.06 (m, 17H), 1.51-1.06 (m, 17H), 1.04-0.78 (m, 2H), 0.76-0.69 (m, 3H), 0.56 (s, 3H).

### Example 45: Synthesis of Compound 214

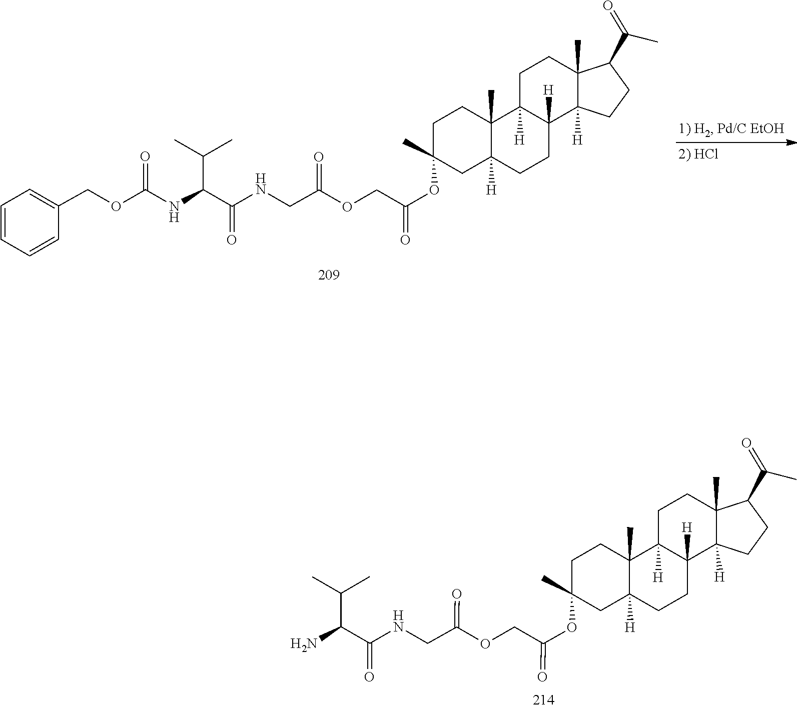

Compound 209 (from Example 29; 97 mg, 0.14 mmol) was dissolved in 10 mL of anhydrous EtOH. The solution was degassed under vacuum, then back filled with nitrogen. This was completed 3 times. To the solution was charged 5% wet Pd/C (98.1 mg, 0.05 mmol). The dark suspension was degassed under vacuum, then back filled with hydrogen. This was completed 3 times and the reaction was allowed to stir overnight under a H₂ balloon. After stirring for 18 h the reaction was diluted with 25 mL of EtOAc and filtered through a Celite plug. The plug was rinsed with additional EtOAc, then concentrated to provide a hazy residue. To the flask containing the residue was dissolved in 2 mL of Et₂O. 2M HCl in Et₂O (100 μL 0.2 mmol) was added to the reaction flask. Upon addition of the HCl, the resulting precipitate was isolated by filtration and washed with 5 mL of Et₂O. The solid was then placed under high vacuum to dry overnight to provide compound 214 (57.3 mg) as a hydrochloride salt. HRMS (ESI+): calculated for C₃₁H₅₁ClN₂O₆ m/z [M+H]⁺: 547.3742, observed 547.3731; ¹H NMR δ: 4.71-4.53 (m, 2H), 4.31-3.92 (m, 3H), 3.25 (br. s., 1H), 2.59-2.48 (m, 1H), 2.28-2.08 (m, 5H), 2.07-1.77 (m, 5H), 1.75-1.35 (m, 14H), 1.33-1.03 (m, 16H), 1.03-0.87 (m, 3H), 0.82-0.69 (m, 4H), 0.61 (s, 3H).

### Example 46: Synthesis of Compound 215

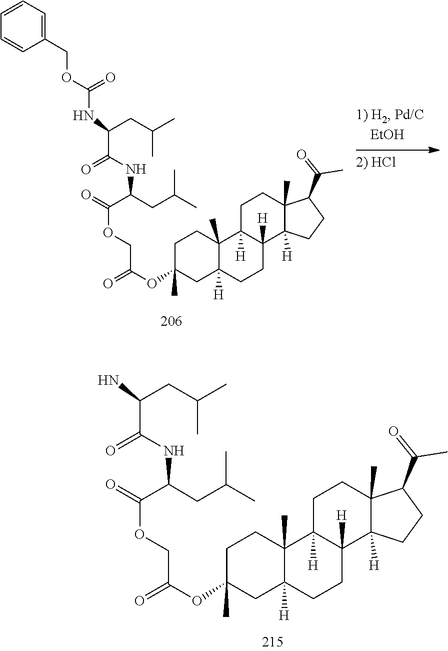

In a similar manner to the procedure outlined in Example 45, substituting Compound 209 with Compound 206 (from Example 22), Compound 215 was obtained. HRMS (ESI+) calculated for C₃₆H₆₁ClN₂O₆ m/z [M+H]⁺: 617.4524, observed 617.4526; ¹H NMR δ: 4.85-4.73 (m, 1H), 4.73-4.59 (m, 2H), 4.58-4.39 (m, 1H), 2.84-2.60 (m, 1H), 2.50 (t, J=8.5 Hz, 1H), 2.30-2.06 (m, 5H), 2.04-1.78 (m, 4H), 1.75-1.53 (m, 7H), 1.52-1.06 (m, 19H), 1.03-0.83 (m, 13H), 0.74 (s, 4H), 0.58 (s, 3H).

### Example 47: Synthesis of Compound 216

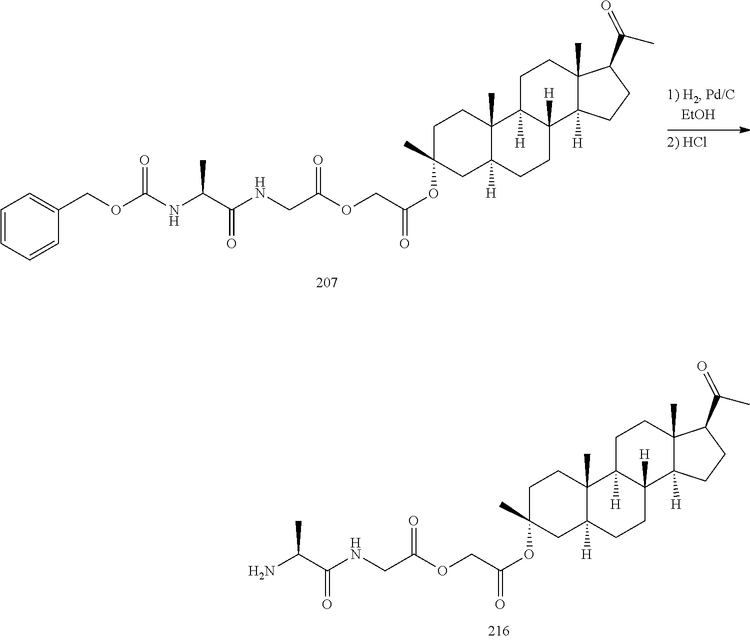

In a similar manner to the procedure outlined in Example 45, substituting Compound 209 with Compound 207 (from Example 23), Compound 216 was obtained. HRMS (ESI+) calculated for C₂₉H₄₇CN₂O₆ m/z [M+H]⁺: 519.3429, observed 519.3418; ¹H NMR (MeOD) δ: 4.59-4.69 (m, 2H), 4.10-4.69 (m, 2H), 4.02 (s, 1H), 3.90-3.95 (m, 1H), 3.05 (q, J=7.03 Hz, 1H), 2.57-2.68 (m, 1H), 1.85-2.27 (m, 7H), 1.12-1.76 (m, 29H). 0.73-1.06 (m, 6H), 0.60 (s, 3H 6).

### Example 48: Synthesis of Compound 128

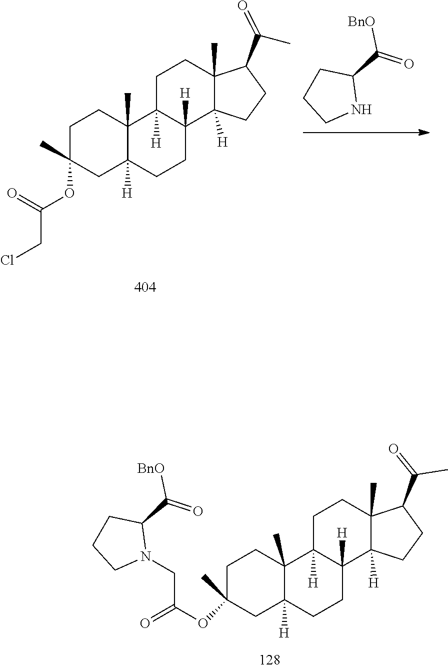

In a similar manner to the procedure outlined in Example 2, substituting 1-(2-methoxyphenyl)-piperazine with L-proline benzyl ester hydrochloride (297 mg, 1.23 mmol) Compound 128 (79.6 mg) was obtained as a solid. HRMS (ESI+): calculated for C₃₆H₅₂NO₅ m/z [M+H]⁺: 578.3840, observed 578.3844; ¹H NMR δ: 7.45-7.19 (m, 5H), 5.28-5.07 (m, 2H), 3.77 (dd, J=4.4, 9.1 Hz, 1H), 3.59-3.44 (m, 2H), 3.23-3.08 (m, 1H), 2.92 (q, J=7.6 Hz, 1H), 2.51 (t, J=8.5 Hz, 1H), 2.31-1.81 (m, 13H), 1.76-1.06 (m, 16H), 1.03-0.67 (m, 6H), 0.60 (s, 3H).

### Example 49: Synthesis of Compound 224

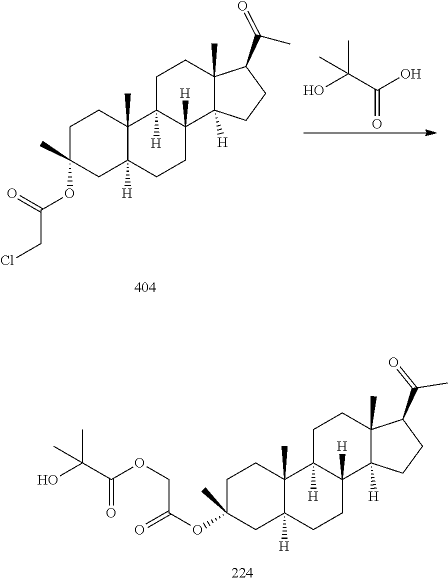

In a similar manner to the procedure outlined in Example 17 (not including the step for forming an HCl salt), substituting 4-morpholineacetic acid with 2-hydroxyisobutyric acid (166 mg, 1.60 mmol), Compound 224 (91.3 mg) was obtained as a foam. HRMS (ESI+): calculated for C₂₈H₄₄NaO₆ m/z [M+H]⁺: 499.3030, observed 499.3027; ¹H NMR δ: 4.63 (s, 2H), 3.00 (s, 1H), 2.60-2.46 (m, 1H), 2.35-1.97 (m, 8H), 1.85 (d, J=11.7 Hz, 1H), 1.75-1.07 (m, 21H), 1.04-0.86 (m, 2H), 0.83-0.68 (m, 5H), 0.61 (s, 3H).

### Example 50: Synthesis of Compound 504

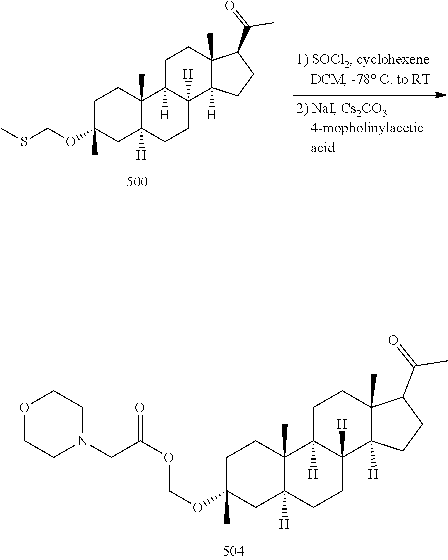

A solution of Compound 500 (from Example 26; 122 mg, 0.31 mmol), cyclohexene (0.5 ml, 4.94 mmol) in DCM was cooled to −78° C. in a dry ice acetone bath. After stirring for 5 minutes, thionyl chloride (22 μL, 0.30 mmol) was added in a single portion and the contents were allowed to slowly warm to ambient temperature overnight. After stirring for 18 h the reaction mixture was concentrated to a residue. The residue was then dissolved in 5 mL of DMF and NaI (62.1 mg, 0.41 mmol) and 4-morpholinylacetic acid (182 mg, 1.25 mmol) were added. The resulting mixture was stirred for 5 minutes, then cesium carbonate (430 mg, 1.32 mmol) was added resulting in a bright yellow slurry. The slurry was heated to 70° C. After stirring for 18 h the reaction was diluted with 25 mL of EtOAc and 15 mL of H₂O to produce a clear biphasic solution. After separation, EtOAc layer was washed with 2×15 mL of saturated NaHCO₃, then 15 mL of saturated NaCl solution. The organic phase was dried using Na₂SO₄, filtered and concentrated to produce an orange residue which was then dried under high vacuum. The crude product was purified by column chromatography using an ISCO™ chromatography system to provide Compound 504 as a solid (47.1 mg). HRMS (ESI+): calculated for C₂₉H₄₈NO₅ m/z [M+H]⁺: 490.3527, observed 490.3527; ¹H NMR δ: 5.44-5.30 (m, 2H), 3.80-3.65 (m, 4H), 3.16-3.12 (s, 2H), 2.59-2.52 (m, 5H), 2.15-1.91 (m, 9H), 1.73-1.49 (m, 8H), 1.46-1.02 (m, 22H), 0.95-0.78 (m, 1H), 0.58-0.53 (m, 4H), 0.54 (s, 3H).

### Example 51: Synthesis of Compound 217

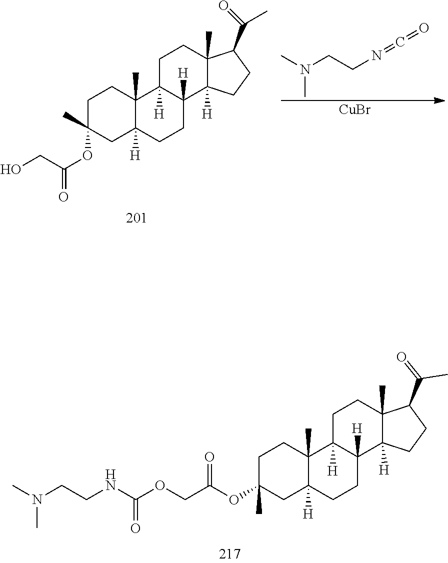

Compound 201 (From Example 25; 75.7 mg, 0.19 mmol) and CuBr (40 mg, 0.18 mmol) were dissolved in 5 mL of DCM to produce a deep green solution. To this solution was added N-(2-isocyanatoethyl)—N, N-dimethylamine (23.4 mg, 0.21 mmol). The reaction was stirred at ambient temperature for 20 h, then diluted with 25 mL of EtOAc, washed with 3×15 ml with H₂O, followed by 15 mL of brine. The washed aqueous was then dried with Na₂SO₄, filtered and concentrated to provide the crude product as a clear oil. The crude product was purified by column chromatography using an ISCO™ chromatography system to provide Compound 217 (free base; 18.7 mg). ¹H NMR δ: 5.71 (br. s., 1H), 4.65-4.41 m, 2H), 3.46-3.30 (m, 3H), 2.67-2.48 (m, 4H), 2.46-2.31 (m, 7H), 2.31-2.07 (m, 6H), 2.05-1.83 (m, 2H), 1.78-1.07 (m, 23H), 1.04-0.075 (m, 5H), 0.59 (s, 3H).

This free base was treated with 2.0 M HCl/diethyl ether (200 uL), and solvent was removed in vacuo to provide Compound 217 as a solid hydrochloride salt (21.9 mg). HRMS (ESI+): calculated for C₂₉H₄₉N₂O₅ m/z [M+H]⁺: 505.3636, observed 505.3624; ¹H NMR δ: 7.01-6.84 (m, 1H), 4.65-4.39 (m, 2H), 3.69 (br. s., 2H), 3.25 (br. s., 2H), 2.90 (br. s, 6H), 2.52 (t, J=8.5 Hz, 1H), 2.79-1.78 (m, 8H), 1.75-1.54 (m, 5H), 1.53-1.07 (m, 22H), 1.03-0.67 (m, 7H), 0.59 (s, 3H).

### Example 52. Synthesis of Compound 407

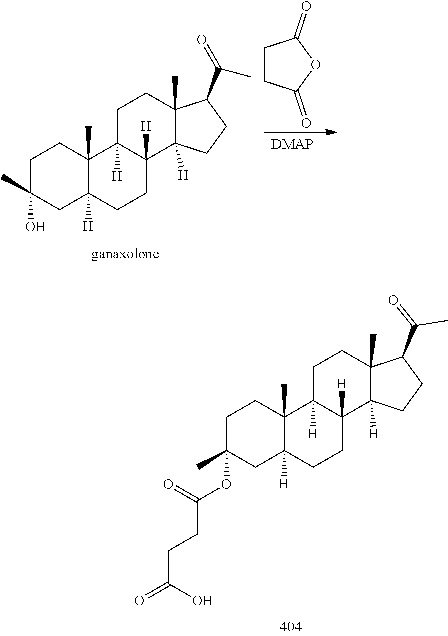

To a solution of ganaxolone (665 mg, 2.0 mmol) in pyridine (4.65 mL) was added succinic anhydride (1.2 g, 12 mmol) and DMAP (244 mg, 2.0 mmol). The reaction vessel was sealed and heated to reflux, which dissolved the reagents. After 42 h, the reaction mixture was cooled to ambient temperature and poured into cold water (95 mL). The pH was adjusted to 3 with 1N HCl and the organic layer was extracted with ethyl aetate (75 mL, 2×). The combined organic layers were washed with brine and dried over sodium sulfate. After filtration, the organic extracts were concentrated and purified via silica gel chromatography using 0-100% ethyl acetate/hexanes to provide Compound 404 as a solid 225 mg. HRMS (ESI+): calculated for C₂₆H₄₀NaO₅ m/z [M+Na]⁺: 455.2768, observed 455.2762; ¹H NMR δ=2.96-2.78 (m, 2H), 2.70-2.53 (m, 1OH), 2.44-2.40 (m, 1H), 2.40-2.36 (m, 1H), 2.25-2.15 (m, 2H), 2.11 (s, 4H), 1.99 (br d, J=11.7 Hz, 1H), 1.94-1.84 (m, 2H), 1.72-1.60 (m, 4H), 1.59-1.49 (m, 2H), 1.48-1.42 (m, 5H), 1.41-1.33 (m, 4H), 1.26 (t, J=7.0 Hz, 5H), 1.22-1.09 (m, 6H), 0.89 (s, 1H), 0.80-0.72 (m, 4H), 0.59 (s, 3H).

### Example 53: Synthesis of Compound 408

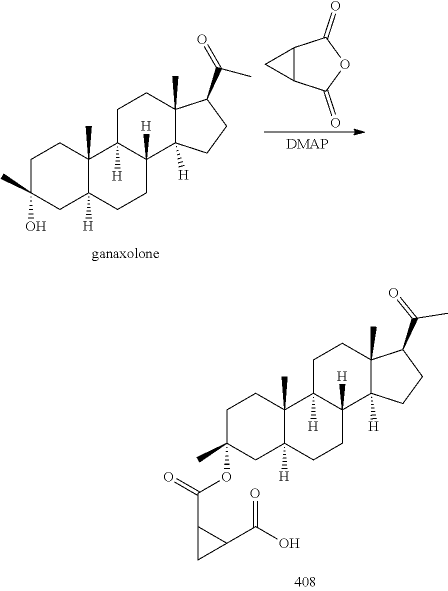

In a similar manner to the procedure outlined in Example 52, substituting succinic anhydride with 3-oxabicyclo[3.1.0]hexane-2,4-dione, Compound 408 was obtained. LCMS (ESI) m/z 467.41 (M+Na)⁺; HRMS (ESI+) calculated for C₂₇H₄₁O₅ m/z [M+H]⁺: 445.2949, observed 445.2950; ¹H NMR CDCl₃ δ: 2.79 (dd, J=2.6, 8.5 Hz, 1H), 2.59-2.45 (m, 1H), 2.22 (br d, J=2.9 Hz, 1H), 2.17 (br s, 1H), 2.30-2.15 (m, 1H), 2.11 (s, 5H), 2.09 (s, 1H), 2.08 (s, 1H), 2.04-1.95 (m, 2H), 1.89 (ddd, J=2.6, 6.6, 11.9 Hz, 2H), 1.74-1.52 (m, 8H), 1.47 (s, 4H), 1.43-1.29 (m, 7H), 1.26 (t, J=7.0 Hz, 6H), 0.90 (s, 1H), 0.83-0.72 (m, 5H), 0.60 (s, 3H).

### Example 54: Synthesis of Compound 420

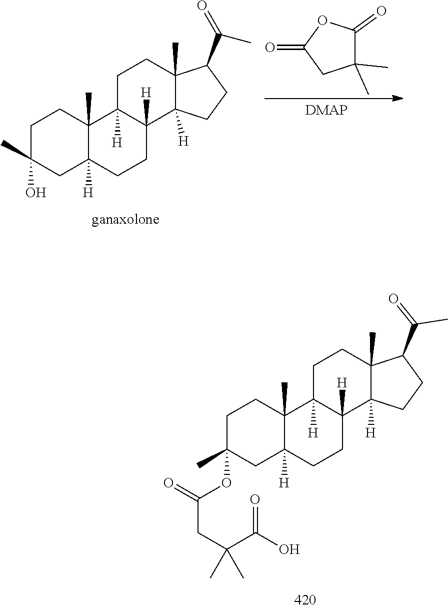

In a similar manner to the procedure outlined in Example 52, substituting succinic anhydride with 2,2-dimethylsuccinic anhydride, Compound 420 was obtained. LCMS (ESI) m/z 483.24 (M+Na)⁺; ¹H NMR δ: 2.62 (s, 1H), 2.60 (d, J=2.9 Hz, 2H), 2.52 (br t, J=8.8 Hz, 2H), 2.27-2.13 (m, 3H), 2.11 (s, 6H), 2.05-1.96 (m, 3H), 1.90 (br d, J=12.9 Hz, 2H), 1.77-1.54 (m, 8H), 1.45 (d, J=1.2 Hz, 7H), 1.39 (br d, J=2.9 Hz, 4H), 1.31 (s, 14H), 1.28-1.16 (m, 13H), 1.02-0.80 (m, 4H), 0.77 (s, 2H), 0.76 (s, 4H), 0.74-0.65 (m, 3H), 0.60 (s, 5H).

### Example 55. Synthesis of Compound 410

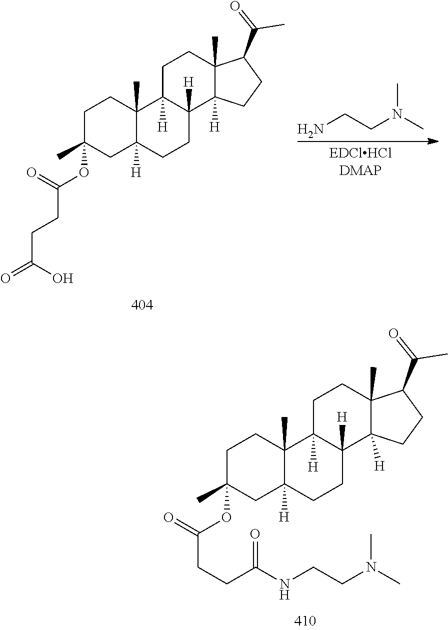

To a solution of Compound 404 (from Example 52; 96 mg, 0.22 mmol) in dichloromethane (1.1 mL), was added sequentially EDCI·HCl (85 mg, 0.44 mmol), 2-dimethylaminoethylamine (0.048 mL, 0.44 mmol), and DMAP (27 mg, 0.22 mmol). The reaction mixture was stirred overnight at ambient temperature. The reaction was diluted with ethyl acetate and 1 N HCl (aq) was added. Brine was added to break up the resulting emulsion and the phases were separated. The aqueous layer was extracted with ethyl acetate (2×) and the combined organic layers were dried over sodium sulfate and filtered. The combined extracts were concentrated and purified via silica gel chromatography using 0-10% methanol/dichloromethane buffered with ammonium hydroxide to give impure material which was purified via reversed-phase preparative HPLC 5-95% acetonitrile/water buffered with trifluoroacetic acid. Lyophilization of the fractions containing product provided Compound 410 as the trifluoroacetic acid salt (53 mg). HRMS (ESI+): calculated for C₃₀H₅₁N₂O₄ m/z [M+H]⁺: 503.3843, observed 503.3856; ¹H NMR (DMSO-d₆) δ=3.42-3.29 (m, 3H), 3.10 (q, J=5.9 Hz, 3H), 2.78 (d, J=4.1 Hz, 7H), 2.53 (s, 1H), 2.45 (d, J=5.9 Hz, 2H), 2.39-2.28 (m, 2H), 2.16-1.89 (m, 7H), 1.79-1.67 (m, 1H), 1.66-1.46 (m, 5H), 1.43-1.19 (m, 11H), 1.12 (br s, 5H), 0.73 (s, 4H), 0.49 (s, 3H).

### Example 56: Synthesis of Compound 409

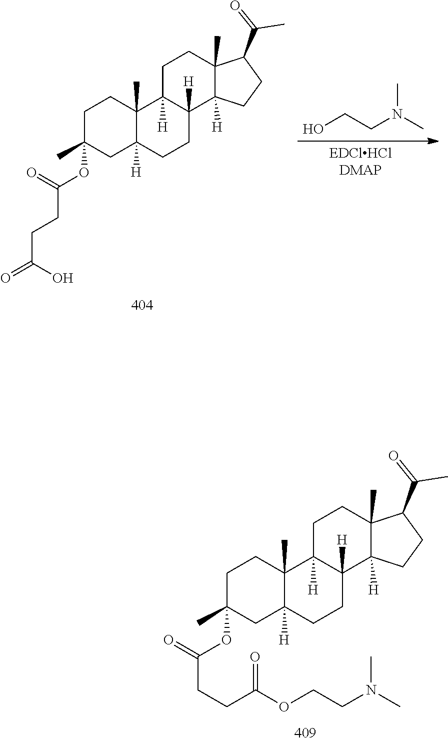

In a similar manner to the procedure outlined in Example 55, substituting dimethylaminoethylamine with 2-dimethylaminoethanol, Compound 409 was obtained as a hydrochloride salt. LCMS (ESI) m/z 504.31 (M+H)⁺; HRMS (ESI+) calculated for C₃₀H₅₀NO₅ m/z [M+H]⁺: 504.3684, observed 504.3690; ¹H NMR DMSO-d₆) δ=4.38-4.27 (m, 3H), 3.36 (br d, J=4.7 Hz, 2H), 2.81 (d, J=4.7 Hz, 5H), 2.54 (td, J=2.6, 4.8 Hz, 4H), 2.09 (br s, 1H), 1.95 (br d, J=11.7 Hz, 1H), 1.80-1.70 (m, 1H), 1.68-1.47 (m, 3H), 1.40 (br s, 1H), 1.38 (s, 3H), 1.33-1.20 (m, 3H), 1.12 (br s, 4H), 0.99-0.78 (m, 1H), 0.74 (s, 2H), 0.71-0.62 (m, 1H), 0.50 (s, 2H).

### Example 57: Synthesis of Compound 411

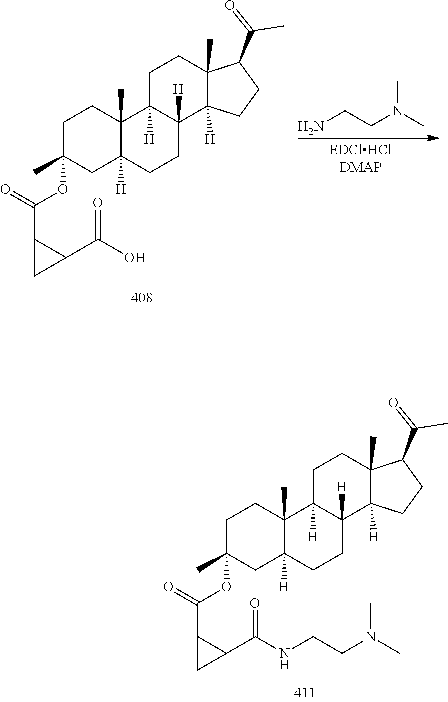

In a similar manner to the procedure outlined in Example 55, substituting Compound 404 with Compound 408 (from Example 53), Compound 411 was obtained as a trifluoroacetate salt. LCMS (ESI) m/z 515.75 (M+H)⁺; HRMS (ESI+) calculated for C₃₁H₅₁N₂O₄ m/z [M+H]⁺: 515.3843, observed 515.3857; ¹H NMR (DMSO-d₆) δ=8.23-8.13 (m, 2H), 6.87 (d, J=7.0 Hz, 2H), 3.11 (s, 6H), 2.80-2.66 (m, 2H), 2.31-2.22 (m, 1H), 2.09-2.02 (m, 4H), 2.01-1.87 (m, 4H), 1.82-1.68 (m, 1H), 1.64-1.47 (m, 4H), 1.34-1.30 (m, 5H), 1.21 (s, 2H), 1.17-1.06 (m, 5H), 0.75-0.68 (m, 4H), 0.49 (s, 3H).

### Example 58: Synthesis of Compound 412

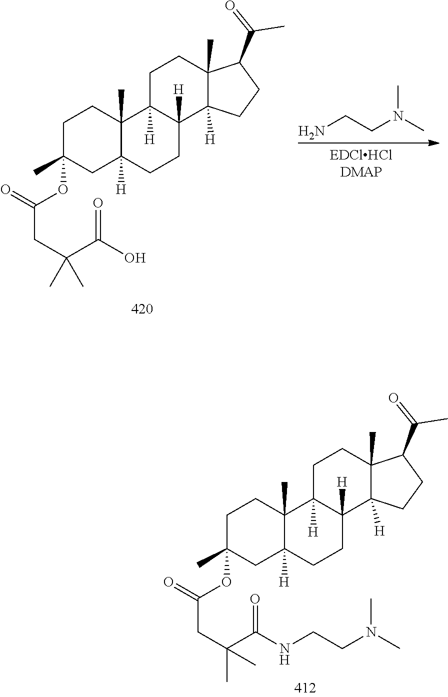

In a similar manner to the procedure outlined in Example 55, substituting Compound 404 with Compound 420 (from Example 54), Compound 412 was obtained as a trifluoroacetate salt. LCMS (ESI) m/z 531.41 (M+H)⁺; HRMS (ESI+) calculated for C₃₂H₅₅N₂O₄ m/z [M+H]⁺: 531.4156, observed 531.4150; ¹H NMR (DMSO-d₆) δ=7.79 (t, J=5.6 Hz, 1H), 3.36 (q, J=5.9 Hz, 3H), 3.10 (q, J=5.9 Hz, 2H), 2.80 (d, J=4.7 Hz, 6H), 2.59-2.50 (m, 1H), 2.02 (br s, 2H), 1.73 (br d, J=11.7 Hz, 1H), 1.67-1.47 (m, 4H), 1.36 (s, 7H), 1.29-1.20 (m, 3H), 1.14 (s, 7H), 0.97-0.80 (m, 1H), 0.73 (s, 3H), 0.70-0.63 (m, 1H), 0.49 (s, 3H).

### Example 59: Synthesis of Compound 413

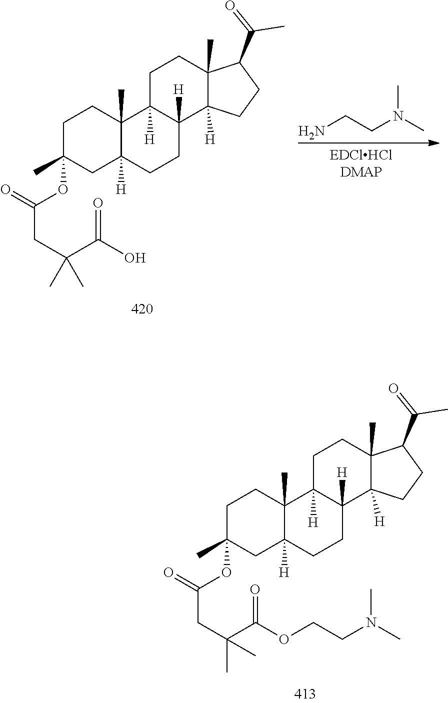

In a similar manner to the procedure outlined in Example 55, substituting Compound 404 with Compound 420 (from Example 54), and substituting dimethylaminoethylamine with 2-dimethylaminoethanol, Compound 413 was obtained as a trifluoroacetate salt. LCMS (ESI) m/z 532.39 (M+H)⁺; HRMS (ESI+) calculated for C₃₂H₅₄NOₛ m/z [M+H]⁺: 532.3997, observed 532.3986; ¹H NMR (DMSO-d6) δ=4.36-4.22 (m, 3H), 3.37 (br s, 3H), 3.15-2.86 (m, 6H), 2.81 (br d, J=4.7 Hz, 7H), 2.76-2.69 (m, 1H), 2.65-2.56 (m, 4H), 2.04 (s, 6H), 1.99-1.91 (m, 2H), 1.77-1.68 (m, 1H), 1.55 (br d, J=10.5 Hz, 6H), 1.37 (s, 8H), 1.31-1.23 (m, 4H), 1.18 (br s, 8H), 1.12 (br s, 4H), 0.73 (s, 3H), 0.49 (s, 3H).

### Example 60. Synthesis of Compound 419

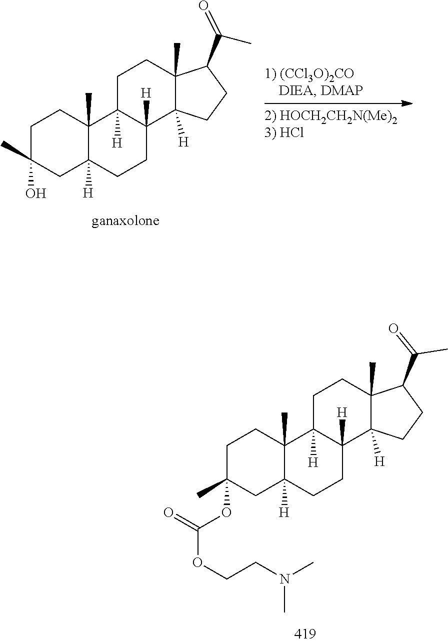

To a solution of ganaxolone (83 mg, 0.25 mmol) and DMAP (37 mg, 0.3 mmol) in dichloromethane (2 mL) under a nitrogen atmosphere at 0° C. was added N,N-diisopropylethylamine (0.174 mL, 1 mmol) followed by dropwise addition a solution of triphosgene (49 mg, 0.165 mmol) in dichloromethane (1.1 mL). The reaction stirred for 3.5 h at this temperature before being cooled to 0° C. and 2-dimethylaminoethylamine (0.13 mL, 1.25 mmol) was added. The reaction was allowed to warm to ambient temperature as it stirred overnight. The reaction was then diluted with water and extracted with ethyl acetate (2×). The combined organic layers were washed with brine and dried over sodium sulfate. The extracts were filtered, concentrated, and purified via silica gel chromatography using 0-10% methanol/dichloromethane to provide Compound 419 which was converted to the hydrochloride salt with HCl (15 mg). HRMS (ESI+): calculated for C₂₇H₄₆NO₄ m/z [M+H]⁺: 448.3421, observed 448.3421; ¹H NMR δ=4.17 (t, J=5.9 Hz, 2H), 2.62 (t, J=5.9 Hz, 2H), 2.56-2.45 (m, 1H), 2.31 (s, 5H), 2.10 (s, 3H), 2.03-1.94 (m, 1H), 1.88 (td, J=2.6, 14.1 Hz, 1H), 1.73-1.53 (m, 4H), 1.46 (s, 3H), 1.44-1.33 (m, 3H), 1.27-1.11 (m, 7H), 1.08 (s, 1H), 1.01-0.80 (m, 2H), 0.76 (s, 4H), 0.59 (s, 3H).

### Example 61. Synthesis of Compound 220

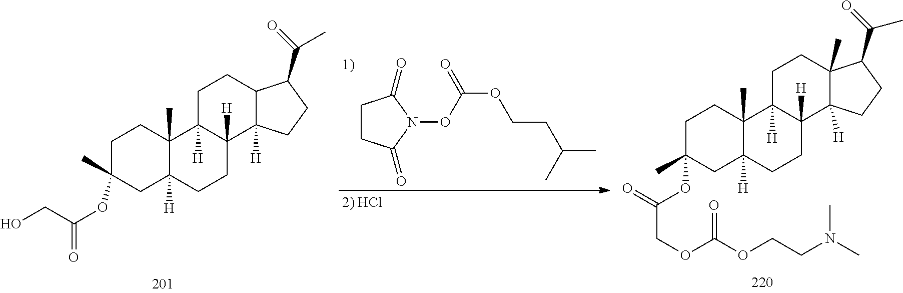

To a solution of di(N-succinimidyl) carbonate (384 mg, 1.5 mmol) in dichloromethane (14 mL) under nitrogen was added, dropwise, 2-dimethylaminoethanol (0.17 mL, 1.65 mmol). The reaction was stirred overnight at ambient temperature to give a 0.11 M solution of N′,N′-dimethylaminoethyl O-succinimidyl carbonate which was used directly in the next step without any purification. LCMS: m/z=252.54 (M+Na)⁺.

To a mixture of Compound 201 (from Example 25; 76 mg, 0.2 mmol) and DMAP (48 mg, 0.39 mmol) under nitrogen was added a 0.11 M solution of solution of N′,N′-dimethylaminoethyl O-succinimidyl carbonate in dichloromethane (5.6 mL) followed by N,N-diisopropylethylamine (0.07 mL, 0.39 mmol). The reaction was stirred overnight under nitrogen at ambient temperature. The mixture was diluted with water and the organic phase was extracted with ethyl acetate (2×). The combined organic layers were washed with brine and dried over sodium sulfate. The extracts were filtered, concentrated, and purified via silica gel chromatography with 0-10% methanol/dichloromethane to provide Compound 220 which was converted to the hydrochloride salt using HCl (36 mg). HRMS (ESI+): calculated for C₂₉H₄₈NO₆ m/z [M+H]⁺: 506.3476, observed 506.3486; ¹H NMR δ=4.55 (d, J=3.5 Hz, 2H), 4.29 (t, J=5.9 Hz, 2H), 2.63 (t, J=5.9 Hz, 2H), 2.56-2.47 (m, 1H), 2.33-2.28 (m, 6H), 2.27-2.15 (m, 1H), 2.11 (s, 3H), 2.03-1.95 (m, 1H), 1.91 (dd, J=2.6, 11.4 Hz, 1H), 1.51-1.46 (m, 4H), 1.45-1.11 (m, 13H), 0.97 (t, J=7.3 Hz, 1H), 0.94-0.85 (m, 1H), 0.82-0.73 (m, 4H), 0.59 (s, 3H).

### Example 62. Synthesis of Compound 222

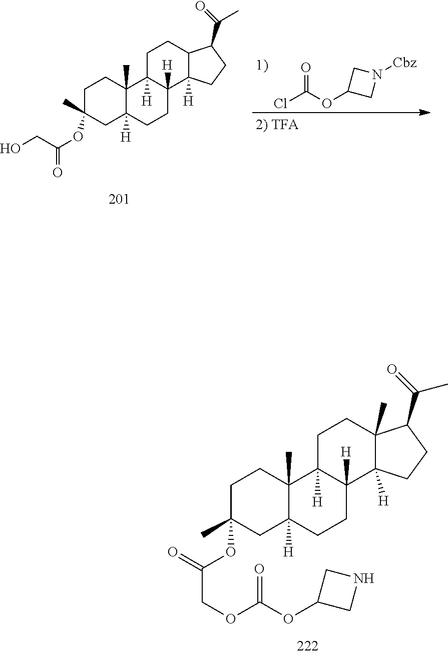

To a solution of Compound 201 (from Example 25, 78 mg, 0.2 mmol) in pyridine under nitrogen cooled to −20° C. was added dropwise a solution of tert-butyl 3-[(chlorocarbonyl)oxy]azetidine-1-carboxylate (235 mg, 1.0 mmol) in dichloromethane (1 mL). The reaction was slowly warmed to ambient temperature over 4 hours as it stirred. The reaction was cooled to 0° C. and quenched with 1 N HCl (aq) and extracted with ethyl acetate (2×). The combined organic layers were washed with brine and dried over sodium sulfate. The extracts were filtered, concentrated, and purified via silica gel chromatography using 0-25% ethyl acetate/hexanes to give an intermediate Cbz-containing compound that was used immediately in the next step (109 mg). LCMS: m/z 612.37 (M+Na)⁺.

To the Cbz containing compound (25 mg, 0.045 mmol) in dichloromethane (0.95 mL) was added trifluoroacetic acid (0.047 mL). The reaction was stirred (2 h) before being concentrated and purified via reversed-phase preparative HPLC. Lyophilization of the fractions containing product provided Compound 111 as a trifluoroacetate salt, 14 mg. HRMS (ESI+): calculated for C₂₈H₄₄NO₆ m/z [M+H]⁺: 490.3163, observed 490.3165; ¹H NMR (DMSO-d₆) δ=5.36-5.09 (m, 1H), 4.68 (s, 2H), 4.31 (br dd, J=5.9, 12.3 Hz, 2H), 4.12-3.93 (m, 2H), 2.71 (quin, J=1.8 Hz, 1H), 2.57-2.53 (m, 2H), 2.42 (t, J=1.8 Hz, 1H), 2.25 (quin, J=1.9 Hz, 1H), 2.13-2.06 (m, 1H), 2.05-2.01 (m, 4H), 2.00-1.91 (m, 2H), 1.87-1.71 (m, 1H), 1.56 (br d, J=5.9 Hz, 4H), 1.53 (s, 2H), 1.41 (s, 3H), 1.31 (br d, J=8.2 Hz, 4H), 1.22 (s, 1H), 1.11 (br d, J=2.9 Hz, 5H), 0.76 (s, 1H), 0.74 (s, 2H), 0.49 (s, 3H).

### Example 63. Synthesis of Compound 414

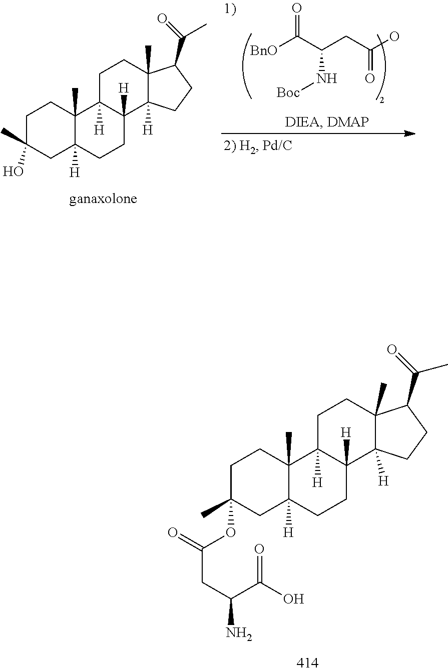

To a solution of N-(tert-butoxycarbonyl) aspartic acid 1-benzyl ester (1.94 g, 6 mmol) in dichloromethane (6 mL) was added a 1M solution of N,N′-dicyclohexylcarbodiimide (3 mL, 3 mmol). The reaction was stirred at ambient temperature overnight before being filtered and concentrated. The resulting oily residue, N-(tert-butoxycarbonyl) 1-benzyl ester 4-aspartic anhydride, was used in the next step without further purification.

To a solution of ganaxolone (100 mg, 0.3 mmol) and DMAP (73 mg, 0.6 mmol) in dichloromethane (3 mL) under nitrogen was added N,N-diisopropylethylamine (0.23 mL, 1.3 mmol) followed by a solution of the N-(tert-butoxycarbonyl) 1-benzyl ester 4-aspartic anhydride (3 mmol) in dichloromethane (3.5 mL). The reaction was stirred overnight under nitrogen at ambient temperature. The reaction was quenched with the addition of saturated aqueous sodium bicarbonate and diluted with dichloromethane and the layers were separated. The organic layer was washed with 1 N HCl (aq) and dried over sodium sulfate. The extract was filtered, concentrated, and purified via silica gel chromatography using 0-25% ethyl acetate to provide an intermediate containing Boc and benzyl ester protecting groups (194 mg) as a solid that was used directly in the next step without further purification. LCMS: m/z=660.48 (M+Na)*.

To a solution of the protected intermediate (190 mg, 0.3 mmol) in methanol (6.0 mL) was added palladium on carbon (35 mg, 10% by mass). The reaction mixture was purged and back-filled with hydrogen (3×) and stirred vigorously overnight. Consumption of starting material was apparent by LC/MS and the reaction mixture was filtered and the filtrate concentrated to give a Boc-protected intermediate that was used directly in the next step.

The Boc-protected intermediate (64 mg, 0.117 mmol) was dissolved in 2 N HCl/diethyl ether (0.58 mL, 1.17 mmol) at ambient temperature. Complete consumption of starting material was apparent by LC/MS after approximately 1 h and the reaction mixture was concentrated. Purification via reversed-phase preparative HPLC using 5-95% acetonitrile buffered with trifluoroacetic acid, and lyophilization, provided Compound 414 as a trifluoroacetate salt, 20 mg. HRMS (ESI+): calculated for C₂₆H₄₂NO₅ m/z [M+H]⁺: 448.3057, observed 448.3050; ¹H NMR (DMSO-d₆) δ=2.99-2.65 (m, 2H), 2.59-2.51 (m, 1H), 2.16-2.06 (m, 1H), 2.03 (s, 3H), 1.97 (t, J=11.7 Hz, 2H), 1.76 (br d, J=11.1 Hz, 1H), 1.67-1.47 (m, 5H), 1.40 (s, 4H), 1.37-1.24 (m, 5H), 1.12 (br s, 6H), 0.98-0.82 (m, 1H), 0.73 (s, 3H), 0.71-0.63 (m, 1H), 0.49 (s, 3H); MS: m/z=448.23 (M+H+).

### Example 64: Synthesis of Compound 416

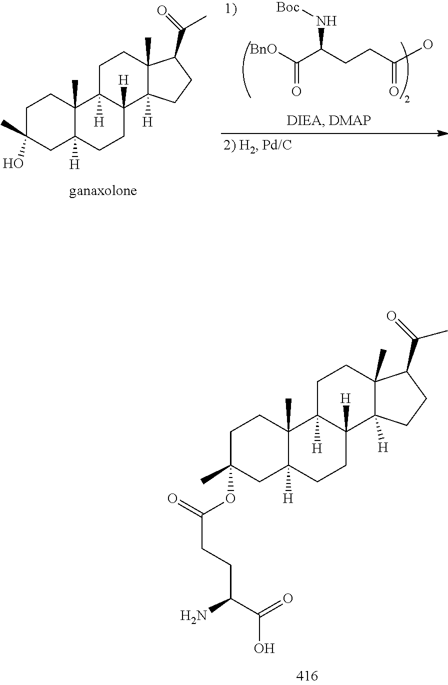

In a similar manner to the procedure outlined in Example 63, substituting N-(tert-butoxycarbonyl) aspartic acid 1-benzyl ester with N-(tert-butoxycarbonyl)-L-glutamic acid 1-benzyl ester, Compound 416 was obtained as a trifluoroacetic acid salt. LCMS (ESI) m/z 484.21 (M+Na)⁺; HRMS (ESI+) calculated for C₂₇H₄₄NO₅ m/z [M+H]⁺: 448.3057, observed 448.3050; ¹H NMR (DMSO-d₆) δ=3.80-3.65 (m, 2H), 3.58 (d, J=11.1 Hz, 1H), 2.85-2.75 (m, 1H), 2.75-2.68 (m, 1H), 2.58-2.52 (m, 2H), 2.46-2.33 (m, 1H), 2.33-2.23 (m, 1H), 2.15-2.06 (m, 2H), 2.05 (s, 1H), 2.04 (s, 3H), 2.00-1.87 (m, 4H), 1.80-1.67 (m, 2H), 1.66-1.48 (m, 5H), 1.39 (s, 5H), 1.37-1.21 (m, 7H), 1.12 (br s, 7H), 0.96-0.86 (m, 1H), 0.86-0.82 (m, 1H), 0.74 (s, 4H), 0.70-0.62 (m, 1H), 0.49 (s, 3H).

### Example 65. Synthesis of Compound 415

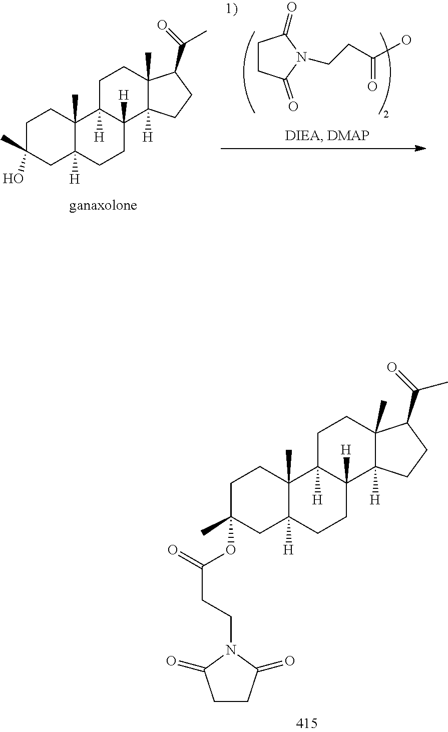

To a solution of 2,5-dioxo-1-pyrrolidinepropanoic acid (465 mg, 2.7 mmol) in dichloromethane (5.4 mL) was added a 1M solution of N,N′-dicyclohexylcarbodiimide (1.35 mL, 1.35 mmol). The reaction was stirred at ambient temperature 3 h before being filtered and the filtrate concentrated. The resulting oily residue, 2,5-dioxo-1-pyrrolidinepropanoic anhydride, was used as-is in the next step.

To a solution of ganaxolone (50 mg, 0.15 mmol) and DMAP (37 mg, 0.3 mmol) in dichloromethane (1.5 mL) under nitrogen was added N,N-diisopropylethylamine (0.12 mL, 0.66 mmol) followed by a solution of 2,5-dioxo-1-pyrrolidinepropanoic anhydride (1.35 mmol) in dichloromethane (1.75 mL). The reaction was stirred overnight under nitrogen at ambient temperature. The reaction was quenched with the addition of 1 N HCl (aq) and diluted with dichloromethane. The layers were separated and the organic layer was washed with brine and dried over sodium sulfate. The extract was filtered, concentrated, and purified via silica gel chromatography using 0-75% ethyl acetate/hexanes to provide Compound 415 as a solid (54 mg). HRMS (ESI+): calculated for C₂₉H₄₃NNaO₅ m/z [M+Na]⁺: 508.3033, observed 508.3035; ¹H NMR δ=3.84-3.75 (m, 2H), 2.72 (s, 4H), 2.66-2.47 (m, 4H), 2.25-2.13 (m, 1H), 2.11 (s, 3H), 2.04-1.94 (m, 1H), 1.93-1.82 (m, 1H), 1.75-1.65 (m, 3H), 1.65-1.62 (m, 2H), 1.57-1.56 (m, 1H), 1.57 (s, 20H), 1.53-1.49 (m, 1H), 1.45 (s, 4H), 1.43-1.32 (m, 4H), 1.30-1.14 (m, 9H), 1.13-1.04 (m, 2H), 0.76 (s, 4H), 0.60 (s, 3H).

### Example 66. Synthesis of Compound 417

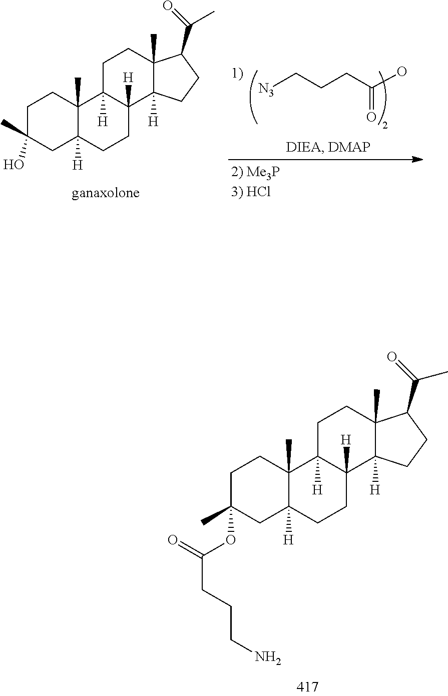

To a solution of 4-azidobutanoic acid (387 mg, 3.0 mmol) in dichloromethane (3.0 mL) was added a 1M solution of N,N′-dicyclohexylcarbodiimide (1.5 mL, 1.5 mmol). The reaction was stirred at ambient temperature for 1 h before being filtered and concentrated. The resulting oily residue, 4-azidobutanoic anhydride, was used as-is in the next step.

To a solution of ganaxolone (50 mg, 0.15 mmol) and DMAP (37 mg, 0.3 mmol) in dichloromethane (1.5 mL) under nitrogen was added N,N-diisopropylethylamine (0.12 mL, 0.66 mmol) followed by a solution of 4-azidobutanoic anhydride (1.5 mmol) in dichloromethane (1.75 mL). The reaction was stirred overnight under nitrogen at ambient temperature. The reaction was quenched with the addition of 1 N HCl (aq) and diluted with dichloromethane. The layers were separated and the organic layer was washed with brine and dried over sodium sulfate. The extract was filtered, concentrated, and purified via silica gel chromatography using 0-25% ethyl acetate to provide 43 mg of an azide-containing intermediate as a solid that was used directly in the next step.

To a solution of the azide-containing intermediate (24 mg, 0.054 mmol) in tetrahydrofuran (0.22 mL) was added dropwise a 1M solution of trimethylphosphine (0.07 mL, 0.07 mmol) in tetrahydrofuran. The reaction mixture was stirred 3.5 h at ambient temperature. Water (1.5 mL) was added and the mixture was stirred 10 min and then diluted with dichloromethane (3 mL). The layers were separated and the organic phase was extracted with dichloromethane (3 mL, 2×). The combined organic layers were dried over sodium sulfate. The extract was filtered, concentrated, and purified via silica gel chromatography using 0-15% methanol/dichloromethane. Concentration of the fractions containing product provided Compound 417 which was converted to the hydrochloride salt (36 mg) using HCl.

### Example 67: Synthesis of Compound 418

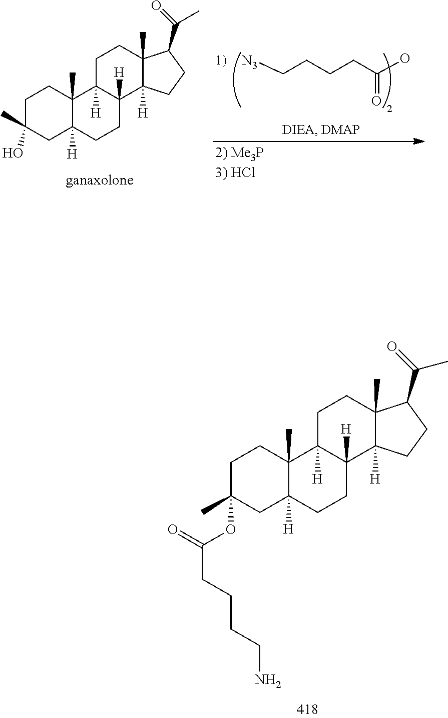

In a similar manner to the procedure outlined in Example 66, substituting 4-azidobutanoic acid with 5-azidopentanoic acid, Compound 418 was obtained as a hydrochloride salt. LCMS (ESI) m/z 432.33 (M+H)⁺; HRMS (ESI+) calculated for C₂₇H₄₆NO₃ m/z [M+H]⁺: 432.3472, observed 432.3471; ¹H NMR δ: 3.43-3.14 (m, 1H), 2.64-2.46 (m, 1H), 2.42-2.25 (m, 8H), 2.24-2.13 (m, 3H), 2.11 (s, 3H), 1.86 (br d, J=11.7 Hz, 10H), 1.79-1.55 (m, 14H), 1.43 (s, 3H), 1.29-1.03 (m, 16H), 0.76 (s, 4H), 0.59 (s, 3H).

### Example 68: Synthesis of Compound 120 (Using Compound 125 as Precursor)

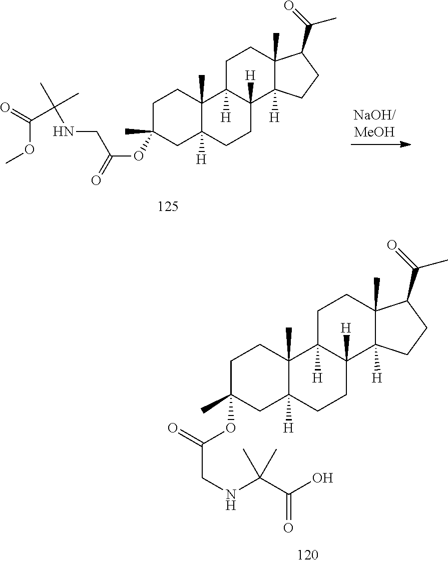

Aqueous NaOH (1M, 250 uL) was added to a solution of Compound 125 (from Example 39, 119.5 mg, 0.244 mmol) and 2.5 mL of MeOH and the resulting solution was allowed to stir at ambient temperature for 2.5 hours. The reaction mixture was then concentrated with a rotary evaporator to remove the bulk of the MeOH, and diluted with 25 mL of EtOAc, then quenched with 5 ml of 1N NaOH. The mixture was transferred to a separatory funnel and the upper organic layer was discarded. The aqueous layer was then acidified with 3.0 mL of 2N HCl to a pH of 1. The aqueous layer was extracted 2×15 mL of DCM. The organic was dried using Na₂SO₄, filtered and concentrated to provide a foam (30.1 mg) containing a 9:1 ratio of Compound 120 to Compound 125, as determined by LCMS. HRMS (ESI+): calculated for C₂₈H₄₆NO₅ m/z [M+H]⁺: 476.337050, observed 476.336738; ¹H NMR δ: 3.84 (br s, 2H) 2.52 (br. s, 1H), 2.27-2.11 (m, 9H), 2.01-1.98 (m, 2H), 1.93-1.82 (m, 1H), 1.79-1.10 (m, 21H), 1.06-0.92 (m, 1H), 0.88-0.72 (m, 6H), 0.60 (2s, 3H).

### Example 69. Synthesis of Compound 127

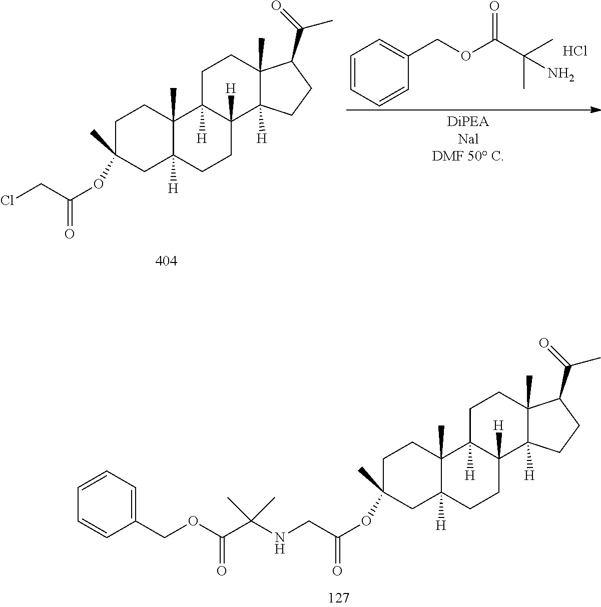

Compound 404 (from Example 1; 496 mg, 1.21 mmol), and sodium iodide (256 mg, 1.71 mmol) were dissolved in anhydrous DMF (8 mL) to produce a clear orange solution. After stirring for 15 minutes, benzyl-2-amino-2-methylpropanoate·HCl (1.35 g, 6.10 mmol) was added followed by DiPEA (2.5 mL, 14.35 mmol) and the resulting mixture was heated to 50° C. After stirring for 22 hours, the reaction mixture was cooled then diluted with 40 mL of EtOAc, then quenched with 35 mL of water. The EtOAc phase was extracted with 2×25 mL of H₂O, then 25 mL of brine. The washed organic phase was dried with Na₂SO₄, filtered and concentrated to provide an oil that was purified by column chromatography using an ISCO™ chromatography system, to provide Compound 127 (375 mg) as an oil. HRMS (ESI+): calculated for C₂₈H₄₄NaO₆ m/z [M+H]⁺: 499.3030, observed 499.3027; ¹H NMR δ: 7.44-7.28 (m, 5H), 5.20-5.11 (m, 2H), 3.35-3.23 (m, 2H), 2.55-2.50 (t, J=8.8 Hz, 1H), 2.22-2.08 (m, 5H), 2.06-1.87 (m, 3H), 1.74-1.07 (m, 26H), 1.02-0.83 (m, 1H), 0.83-0.66 (m, 4H), 0.60 (m, 3H).

### Example 70. Synthesis of Compound 120 (Using Compound 127 as a Precursor)

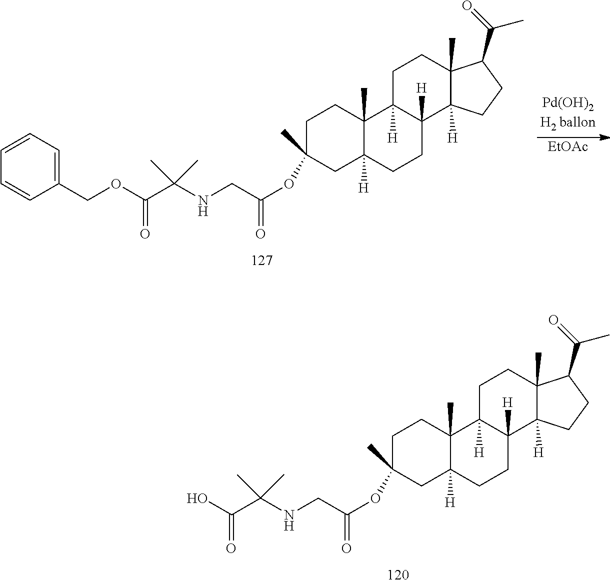

Compound 127 (from Example 69; 257 mg, 0.45 mmol) was dissolved in EtOAc (10 mL) and 20 wt % Pd(OH)₂ (27.6 mg, 0.0393 mmol) to produce a black slurry. The reaction was evacuated, then back filled with H₂. This was repeated 3 times, then the reaction was allowed to stir under a H₂ atmosphere until complete. The reaction mixture was filtered through a plug of CELITE® and rinsed with MeOH (3×15 mL). The solution was concentrated and dried to provide Compound 120 as a solid (190.7 mg). HRMS (ESI+): calculated for C₂₈H₄₆NO₅ m/z [M+H]⁺: 476.337050, observed 476.336738; ¹H NMR δ: 3.85-3.69 (m, 2H), 2.52 (t, J=8.5 Hz, 1H), 2.27-2.07 (m, 6H), 2.05-1.95 (m, 1H), 1.91-1.81 (m, 1H), 1.75-1.05 (m, 28H), 1.04-0.85 (m, 1H), 0.84-0.71 (m, 4H), 0.60 (s, 3H).

### Example 71. Synthesis of Compound 130

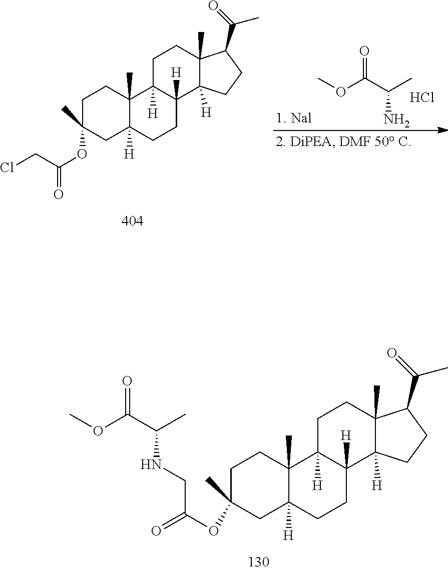

Compound 130 was prepared following a similar procedure as outlined in Example 69, using L-alanine methyl ester·HCl (279 mg, 2.0 mmol) instead of benzyl-2-amino-2-methylpropanoate·HCl. Compound 130 was obtained as a solid (98.7 mg). ¹H NMR δ: 3.75 (s, 3H), 3.39-3.32 (m, 3H), 2.59-2.46 (m, 1H), 2.28-1.79 (m, 11H), 1.67-1.06 (m, 18H), 1.01-0.86 (m, 2H), 0.77-0.72 (m, 4H), 0.61 (s, 3H).

### Example 72. Synthesis of Compound 131

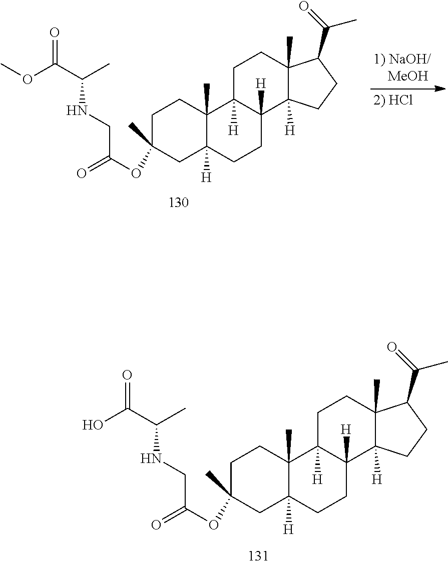

1N NaOH (1 mL) was added dropwise to a solution of Compound 130 (from Example 71; 99 mg, 0.21 mmol) in MeOH (5 mL). The reaction was concentrated to remove a significant portion of the MeOH. The mixture was transferred to a separatory funnel and diluted with 5 mL of 1N NaOH. The mixture was then extracted DCM (2×5 mL). The extractions formed emulsions that took time to split. The organic layers were combined, dried using Na₂SO₄, filtered and then concentrated. Compound 131 was obtained as an oil (20 mg). HRMS (ESI+): calculated for C₂₆H₄₄N₃O₄ m/z [M+H]⁺: 462.332633, observed 462.331726; ¹H NMR δ: 3.99-3.80 (m, 3H), 2.51 (t, J=8.8 Hz, 1H), 2.29-2.06 (m, 6H), 2.04-1.94 (m, 2H), 1.87 (d, J=11.1 Hz, 1H), 1.73-1.06 (m, 23H), 1.04-0.86 (m, 1H), 0.75 (m, 4H), 0.59 (s, 3).

### Example 73. Synthesis of Compound 132

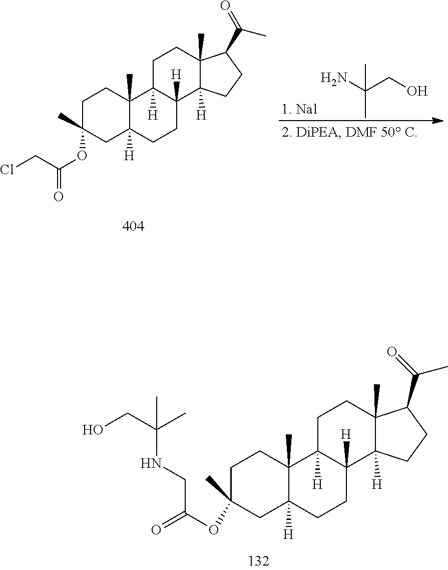

Compound 132 was prepared following a similar procedure as outlined in Example 69, using 2-amino-2-methyl-1-propanol (203 mg, 2.28 mmol) instead of benzyl-2-amino-2-methylpropanoate·HCl. Compound 132 was obtained as a solid (110.8 mg). HRMS (ESI+): calculated for C₂₈H₄₈NO₄ m/z [M+H]⁺: 462.357787, observed 462.358015; ¹H NMR δ: 3.31-3.24 (m, 2H), 3.23-3.17 (m, 2H), 2.48 (t, J=8.8 Hz, 1H), 2.22-2.02 (m, 5H), 2.01-1.83 (m, 2H), 1.71-1.51 (m, 4H), 1.50-0.98 (m, 23H), 0.97-0.81 (m, 4H), 0.55 (s, 3H).

### Example 74. Synthesis of Compound 133

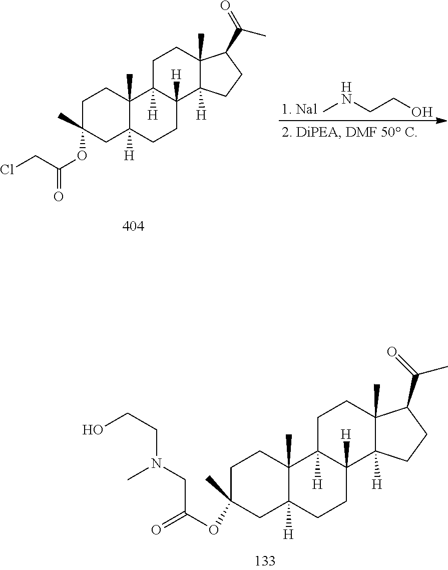

Compound 133 was prepared following a similar procedure as outlined in Example 69, using 2-(methylamino)ethanol (0.16 mL, 2.13 mmol) instead of benzyl-2-amino-2-methylpropanoate·HCl. Compound 133 was obtained as an oil (118.9 mg). HRMS (ESI+): calculated for C₂₇H₄₆NO₄ m/z [M+H]⁺: 448.342135, observed 448.342812; ¹H NMR δ: 3.55 (d, J=4.7 Hz, 2H), 3.42-3.21 (m, 2H), 3.13 (d, J=13.5 Hz, 1H), 2.77-2.60 (m, 2H), 2.58-2.32 (m, 3H), 2.25-1.84 (m, 5H), 1.75-1.00 (m, 14H), 0.87 (dt, J=4.7, 11.7 Hz, 1H), 0.72-0.58 (m, 3H), 0.57-0.36 (m, 3H).

### Example 75. Synthesis of Compound 309

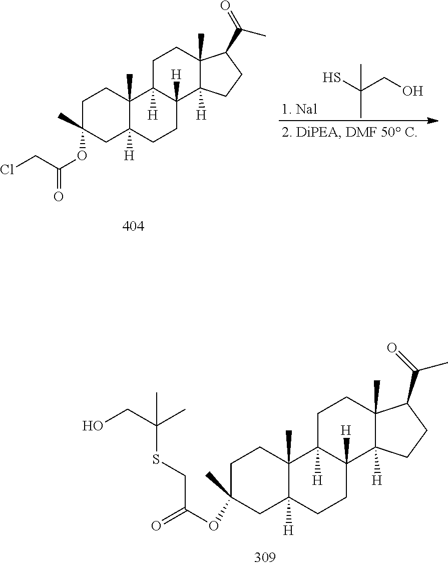

Compound 309 was prepared following a similar procedure as outlined in Example 69, using 2-methyl-2-sulfanylpropan-1-ol (0.25 mL, 2.35 mmol) instead of benzyl-2-amino-2-methylpropanoate·HCl. Compound 309 was obtained as an oil (172.6 mg). HRMS (ESI+): calculated for C₂₈H₄₇O₄S m/z [M+H]⁺: 479.318957, observed 479.318148; ¹H NMR δ: 3.32 (br. s. 2H), 3.27 (br. s. 1H), 3.19 (d, J=1.2 Hz, 2H), 2.56-2.45 (m, 1H), 2.24-2.06 (m, 5H), 2.04-1.87 (m, 2H), 1.73-1.53 (m, 4H), 1.52-1.07 (m, 21H), 0.99-0.83 (m, 1H), 0.79-0.65 (m, 4H), 0.57 (s, 3H).

### Example 76. Synthesis of Compound 134

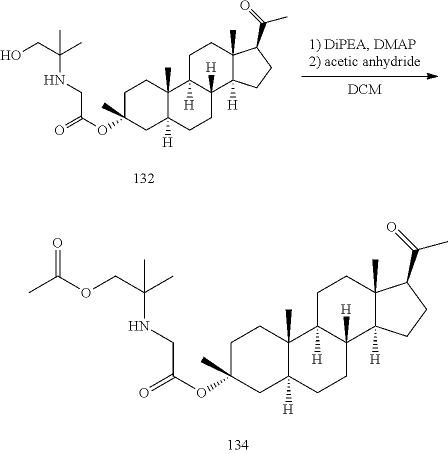

Acetic anhydride (0.023 mL, 0.24 mmol) was added to a solution of Compound 132 (from Example 73; 104 mg, 0.23 mmol), DMAP (3 mg, 0.03 mmol) and DiPEA (0.04 mL, 0.23 mmol) in DCM (5 mL), and the mixture was stirred at ambient temperature overnight. Water (10 mL) was added and the mixture was stirred for 15 minutes. The mixture was concentrated to remove the DCM, then acidified by the addition of 2 mL of 2N HCl. The mixture was stirred for 5 minutes, then transferred to a separatory funnel and extracted with EtOAc (2×25 mL). The combined EtOAc extracts were dried with Na₂SO₄, filtered and concentrated to provide Compound 134 (102.5 mg) as a oil. HRMS (ESI+): calculated for C₃₀H₅₀NO₅ m/z [M+H]⁺: 504.368350, observed 504.368827; ¹H NMR δ: 6.02 (br. s. 1H), 4.03-3.93 (m, 2H), 3.51-3.36 (m, 2H), 2.48 (t, J=8.8 Hz, 1H), 2.20-2.07 (m, 9H), 2.02-1.83 (m, 4H), 1.69-1.03 (m, 24H), 0.99-0.80 (m, 1H), 0.77-0.67 (m, 4H), 0.55 (s, 3H).

### Example 77. Synthesis of Compound 135

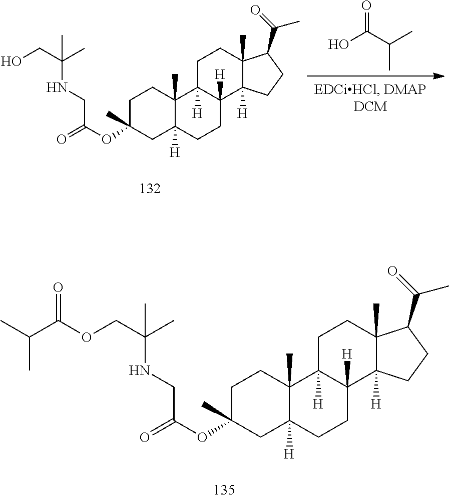

EDCi·HCl (79 mg, 0.41 mmol) was added to a solution of Compound 132 (from Example 73; 116 mg, 0.25 mmol), isobutyric acid (0.04 mL, 0.45 mmol), DMAP (6 mg, 0.05 mmol) in DCM (3 mL) and the reaction mixture was stirred overnight at ambient temperature. The reaction was worked up as follows: add 5 mL of H₂O and stir for 15 minutes. The reaction mixture was diluted with DCM (10 mL) and the aqueous phase was separated and discarded. The DCM phase was washed with saturated NaCl solution, dried (Na₂SO₄), filtered and concentrated. The crude product was purified by column chromatography using an ISCO™ chromatography system to provide Compound 135 (97 mg) as an oil. HRMS (ESI+): calculated for C₃₂H₅₄NO₅ m/z [M+H]⁺: 532.399650, observed 532.398550; ¹H NMR δ: 3.93-3.86 (m, 2H), 3.33-3.26 (m, 2H), 2.64-2.43 (m, 2H), 2.21-2.03 (m, 5H), 2.01-1.83 (m, 3H), 1.70-1.50 (m, 4H), 1.49-1.01 (m, 27H), 0.98-0.80 (m, 1H), 0.77-0.64 (m, 4H), 0.56 (s, 3H).

### Example 78. Synthesis of Compound 136

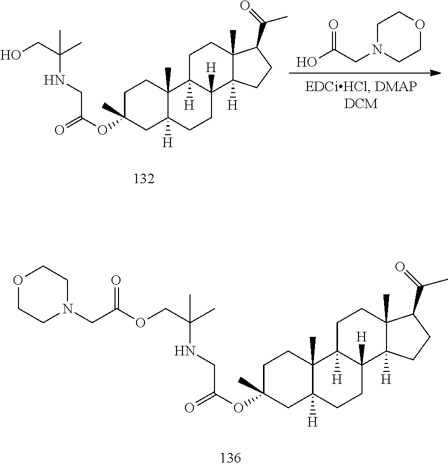

Following a similar procedure as in Example 77, except using morpholin-4-yl-acetic acid (29 mg, 0.20 mmol) instead of isobutyric acid, Compound 136 (172.6 mg) was obtained as a solid. HRMS (ESI+): calculated for C₃₄H₅₇N₂O₆ m/z [M+H]⁺: 589.421114, observed 589.420110; ¹H NMR δ: 4.03-3.91 (m, 2H), 3.81-3.69 (m, 4H), 3.30 (s, 2H), 3.25 (2, 2H), 2.64-2.46 (m, 5H), 2.23-2.06 (m, 5H), 2.05-1.85 (m, 3H), 1.73-1.04 (m, 25H), 1.02-0.84 (m, 1H), 0.81-0.67 (m, 4H), 0.58 (s, 3H).

### Example 79. Synthesis of Compound 310

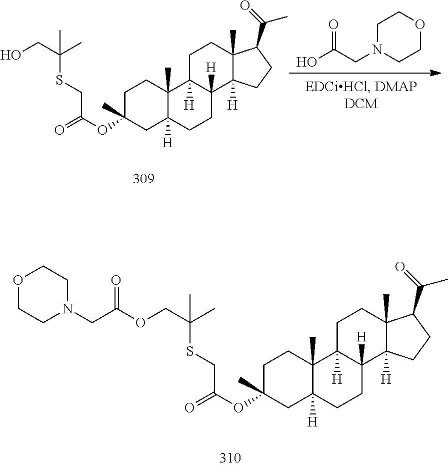

Following a similar procedure as in Example 77, except using morpholin-4-yl-acetic acid (29 mg, 0.20 mmol) instead of isobutyric acid, and using Compound 309 (from Example 75; 60 mg, 0.13 mmol) instead of Compound 132, Compound 310 (42.0 mg) was obtained as a solid. HRMS (ESI+): calculated for C₃₄H₅₆NO₆S m/z [M+H]⁺: 606.382286, observed 606.382306; ¹H NMR δ: 4.13-4.06 (m, 2H), 3.77-3.66 (m, 6H), 3.29-3.20 (m, 2H), 2.63-2.43 (m, 5H), 2.21-2.10 (m, 2H), 2.08 (s, 3H), 2.02-1.85 (m, 2H), 1.70-1.53 (m, 4), 1.51-1.09 (m, 26H), 0.98-0.81 (m, 1H), 0.79-0.67 (m, 4H), 0.57 (s, 3H).

### Example 80. Synthesis of Compound 225

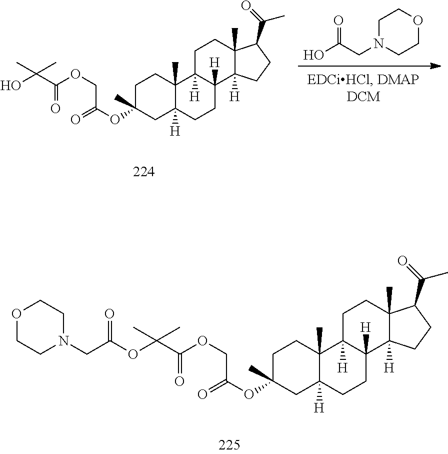

EDCi·HCl (122 mg, 0.64 mmol) was added to a solution of Compound 224 (from Example 49; 40 mg, 0.08 mmol), morpholin-4-yl-acetic acid (86 mg, 0.59 mmol), and DMAP (5 mg, 0.04 mmol) in DCM (3 mL) and the reaction mixture was stirred overnight at ambient temperature. Water (5 mL) was added and the mixture was stirred for 15 minutes. The mixture was transferred to a separatory funnel, diluted with 10 mL of DCM, shaken and the layers were separated. The upper aqueous layer was discarded and the lower organic layer was washed with saturated NaCl solution. The organic layer was then dried with Na₂SO₄, filtered and concentrated using a rotatory evaporator. The crude material was purified by column chromatography using an ISCO™ chromatography system to afford Compound 225 (25.2 mg) as a solid. HRMS (ESI+): calculated for C₃₄H₅₄NO₈ m/z [M+H]⁺: 604.384394, observed 604.384184; ¹H NMR δ: 4.65-4.48 (m, 2H), 3.83-3.69 (m, 4H), 3.32-3.20 (m, 2H), 2.64-2.57 (m, 4H), 2.53 (t, J=9.1 Hz, 1H), 2.30-2.10 (m, 6H), 2.00 (d, J=11.7 Hz, 1H), 1.92-1.81 (m, 2H), 1.77-1.09 (m, 24H), 1.03-0.83 (m, 1H), 0.83-0.76 (m, 4H), 0.60 (s, 3H).

### Example 81. Synthesis of Compound 137

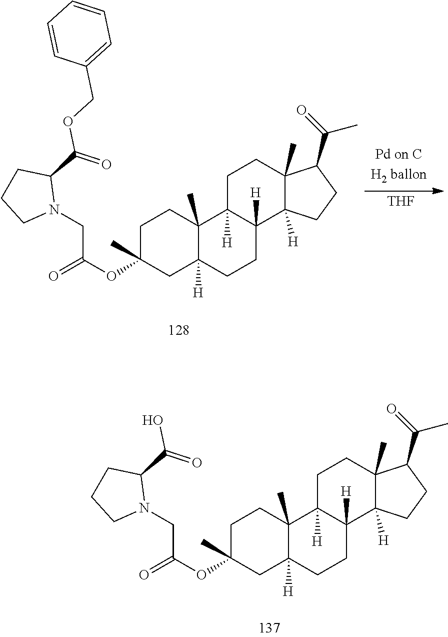

To a solution of Compound 128 (from Example 48; 54 mg, 0.09 mmol) in EtOAc (10 mL) was added 5 wt % wet Pd(C) (29 mg, 0.014 mmol) to produce a black slurry. The reaction was evacuated, then back filled with H₂. This was repeated 3 times, then the reaction was allowed to stir under a H₂ atmosphere until complete. The reaction was worked up as follows: filter the reaction through a plug of CELITE® to remove the catalyst. Rinse the flask with 3×15 mL of MeOH and run it through the CELITE® plug. The filtrate was concentrated to provide Compound 137 as an oil (17.4 mg). HRMS (ESI+): calculated for C₂₉H₄₆NO₅ m/z [M+H]⁺: 488.337050, observed 488.334346; ¹H NMR δ: 4.08 (s, 2H), 3.80 (br. s. 1H), 3.22 (d, J=8.2 Hz, 1H), 2.62-2.41 (m, 3H), 2.38-1.79 (m, 12H), 1.75-1.10 (m, 18H), 0.97 (m, 1H), 0.85-0.70 (m, 4H), 0.60 (s, 3H).

### Example 82. Synthesis of Compound 226

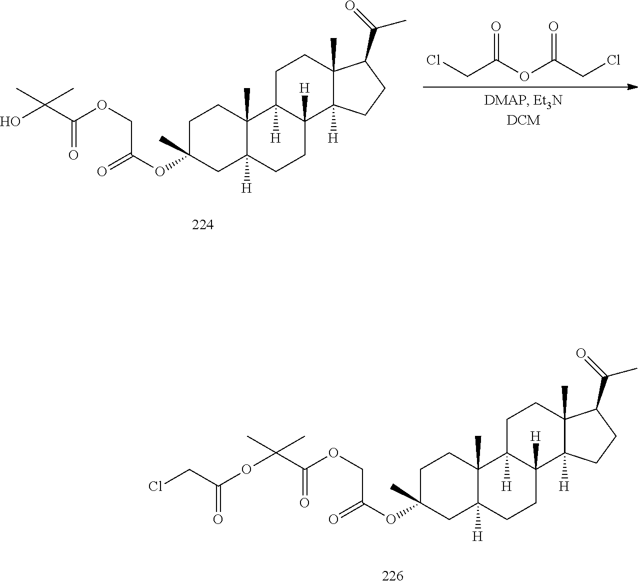

DMAP (5 mg, 0.04 mmol) was added to a solution of Compound 224 (from Example 49; 0.31 g, 0.66 mmol) in DCM (5 mL). After stirring for 10 minutes, Et₃N (0.6 mL, 4.3 mmol) was added dropwise over 1 minute. The resulting solution was stirred at ambient temperature for 5 mins. A solution of chloroacetic anhydride (632 mg, 3.69 mmol) in 5 mL of DCM (5 mL) was then added to the reaction mixture via a syringe. The addition took 2 min to complete and the reaction mixture was allowed to stir for 3 hours at ambient temperature. The reaction was quenched with 15 mL of 1M HCl and then stirred for 10 minutes. The reaction mixture was transferred to a 60 mL separatory funnel and the phases were separated. The lower organic phase was washed with 15 mL of HCl, then 15 ml of H₂O. The washed organic phase was dried with Na₂SO₄, filtered and concentrated, and the concentrate was purified by column chromatography using an ISCO™ chromatography system to provide Compound 226 (330.8 mg) as an oil. ¹H NMR δ: 4.64-4.48 (m, 2H), 4.10-4.04 (m, 2H), 2.54 (t, J=8.8 Hz, 1H), 2.33-2.07 (m, 5H), 2.05-1.97 (m, 1H), 1.91-1.81 (m, 1H), 1.75-1.09 (m, 25H), 1.05-0.86 (m, 1H), 0.84-0.70 (m, 4H), 0.61 (s, 3H).

### Example 83. Synthesis of Compound 227

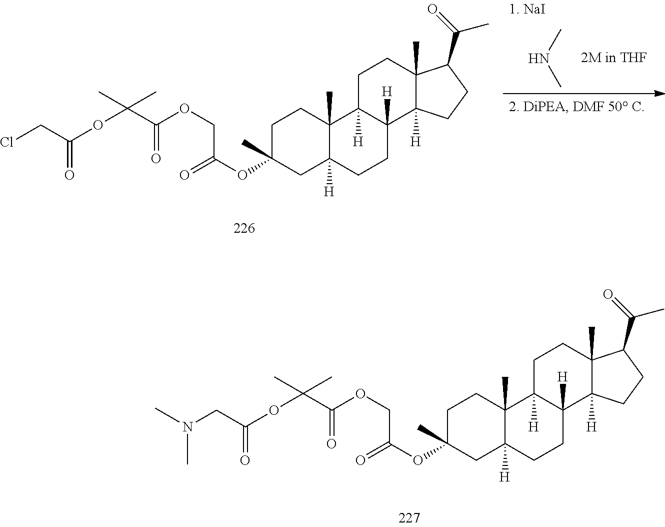

Dimethylamine 2M in THE (0.24 mL, 0.48 mmol) followed by DiPEA (0.1 mL, 0.57 mmol) was added to a solution of Compound 226 (from Example 82; 52 mg, 0.09 mmol) and sodium iodide (90 mg, 0.60 mmol) in DMF (4 mL) and the reaction mixture was stirred at 50° C. for 17 hours. The reaction was diluted with 25 mL of EtOAc, then quenched with 25 mL of water. It was transferred to a separatory funnel and the layers were separated. The EtOAc extract was washed with 2×25 mL of 1N HCl, followed by 15 mL of NaCl. The washed organic phase was then dried with Na₂SO₄, filtered and concentrated, and the concentrate was purified by column chromatography using an ISCO™ chromatography system to provide Compound 227 (39.8 mg) as an oil. HRMS (ESI+): calculated for C₃₂H₅₂NO₇ m/z [M+H]⁺: 562.373829, observed 562.372967; ¹H NMR CDCl3 300 MHz δ: 466-4.47 (m, 2H), 3.26-3.14 (m, 2H), 2.59-2.48 (m, 1H), 2.35 (s, 6H), 2.31-2.08 (m, 5H), 1.99 (d, J=11.7 Hz, 1H), 1.91-1.80 (m, 1H), 1.75-1.54 (m, 1OH), 1.53-1.11 (m, 16H), 1.04-0.85 (m, 1H), 0.82-0.71 (m, 4H), 0.59 (s, 3H).

### Example 84. Synthesis of Compound 228

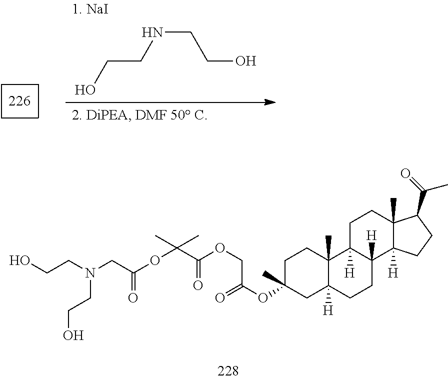

Diethanolamine (0.1 mL, 1.04 mmol) followed by DiPEA (0.180 mL, 1.03 mmol) was added to a solution of Compound 226 (from Example 82; 96 mg, 0.17 mmol) and sodium iodide (89 mg, 0.49 mmol) in anhydrous DMF (4 mL) and the reaction mixture was stirred at 50° C. for 2 hours. The reaction was diluted with 25 mL of EtOAc, then quenched with 25 mL of water. It was transferred to a separatory funnel and the layers were separated. The EtOAc extract was washed with 2×25 mL of 1N HCl, followed by 15 mL of NaCl. The washed organic phase was then dried with Na₂SO₄, filtered and concentrated, and the concentrate was purified by column chromatography using an ISCO™ chromatography system to provide Compound 228 (18.5 mg) as an oil. HRMS (ESI+): calculated for C₃₄H₅₆NO₉ m/z [M+H]⁺: 622.394959, observed 622.394161; ¹H NMR CDCl₃ 300 MHz δ: 4.70-4.49 (m, 2H), 3.60 (t, J=5.0 Hz, 4H), 3.48 (s, 2H), 2.91-2.78 (m, 4H), 2.53 (t, J=8.8 Hz, 1H), 2.34-2.08 (m, 5H), 2.00 (d, J=11.7 Hz, 1H), 1.85 (d, J=12.9 Hz, 1H), 1.74-1.56 (m, 8H), 1.55-1.08 (m, 19H), 1.05-0.84 (m, 2H), 0.84-0.70 (m, 4H), 0.60 (s, 3H).

### Example 85. Synthesis of Compound 138

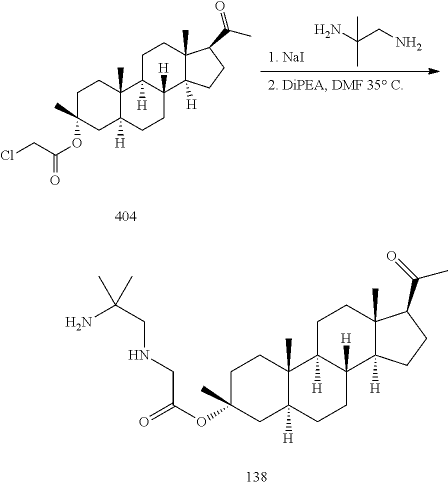

Compound 404 (150 mg, 0.37 mmol) and sodium iodide (176 mg, 1.18 mmol) were added to a 40 mL capped vial with stir bar. The solids were dissolved with anhydrous DMF (4 mL) and stirred at ambient temperature for 15 minutes, then DiPEA (750 μL, 4.31 mmol) was added in a single portion followed by 1,2-diamino-2-methylpropane (190 μL, 1.86 mmol) and the resulting mixture was heated to 35° C. After stirring for 22 hours the reaction mixture was cooled and diluted with ethyl acetate (40 mL), then quenched with water (35 mL). The organic layer was washed with water (2×25 mL) then brine (25 mL), dried with Na₂SO₄ for 30 minutes, filtered and concentrated to provide crude product that was purified by column chromatography using an ISCO™ chromatography system to provide Compound 138 (69 mg) as an oil. HRMS (ESI+): calculated for C₂₈H₄₉N₂O₃ m/z [M+H]⁺: 461.3733, observed 461.3738; ¹H NMR δ: 3.42-3.30 (m, 2H), 3.12 (br. s. 4H), 2.62-2.42 (m, 3H), 2.26-2.03 (m, 5H), 2.02-1.82 (m, 2H), 1.71-1.00 (m, 22H), 0.98-0.80 (m, 2H), 0.78-0.66 (m, 5H), 0.61-0.51 (s, 3H).

### Example 86. Synthesis of Compound 140

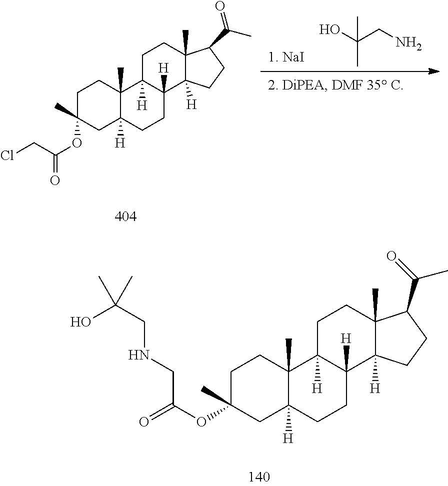

Following a similar procedure as in Example 85, but using 1-amino-2-methylpropan-2-ol instead of 1,2-diamino-2-methylpropane, Compound 140 (164.2 mg) was obtained as an oil. HRMS (ESI+): calculated for C₂₈H₄₈NO₄ m/z [M+H]⁺: 462.3578, observed 462.3573; ¹H NMR δ: 3.38-3.28 (m, 2H), 2.56-2.39 (m, 3H), 2.22-2.00 (m, 5H), 1.99-1.79 (m, 2H), 1.69-0.99 (m, 1.69-0.99 (m, 27H), 0.96-0.76 (m, 1H), 0.74-0.59 (m, 4H), 0.53 (s, 3H).

### Example 87. Synthesis of Compound 232

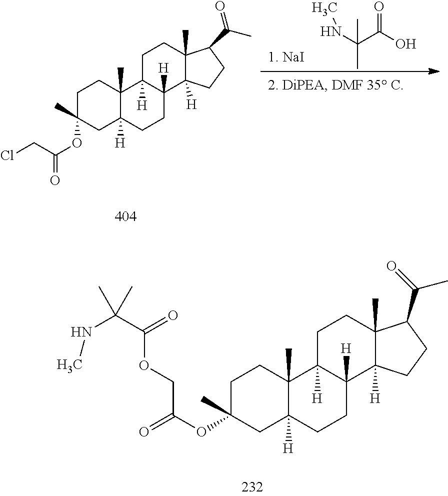

Following a similar procedure as in Example 85, but using 2-(methylamino)isobutyric acid (215 mg, 2.01 mmol) instead of 1,2-diamino-2-methylpropane, Compound 232 (48.8 mg) was obtained as an oil. HRMS (ESI+): calculated for C₂₉H₄₈NO₅ m/z [M+H]⁺: 490.3527, observed 490.3523; ¹H NMR δ: 4.64-4.47 (m, 2H), 2.57-2.46 (m, 1H), 2.38-2.30 (m, 3H), 2.29-2.20 (m, 1H), 2.18-1.93 (m, 7H), 1.90-1.81 (m, 1H), 1.73-1.05 (m, 24H), 1.02-0.83 (m, 1H), 0.81-0.68 (m, 4H), 0.58 (s, 3H).

### Example 88. Synthesis of Compound 244

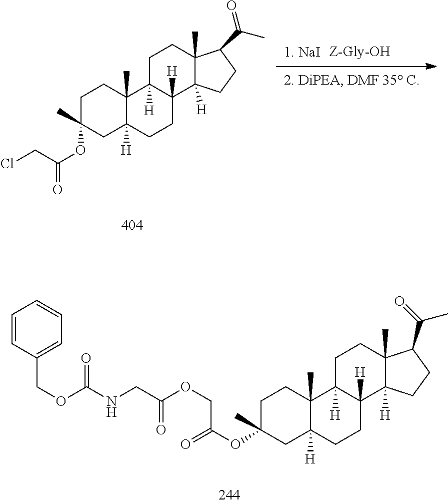

Following a similar procedure as in Example 85, but using Z-Gly-OH instead of 1,2-diamino-2-methylpropane, Compound 244 (135 mg) was obtained as a foam. ¹H NMR δ: 7.44-7.30 (m, 5H), 5.26 (br. s. 1H), 5.15 (s, 2H), 4.69-4.55 (m, 2H), 4.19-4.07 (m, 2H), 2.50 (t, J=8.8 Hz, 1H), 2.28-2.07 (m, 5H), 2.05-1.96 (m, 1H), 1.89 (dd, J=2.9 Hz, 11.1 Hz, 1H), 1.76-1.05 (m, 19H), 1.03-0.87 (m, 1H), 0.82-0.72 (m, 4H), 0.60 (S, 3H).

### Example 89. Synthesis of Compound 247

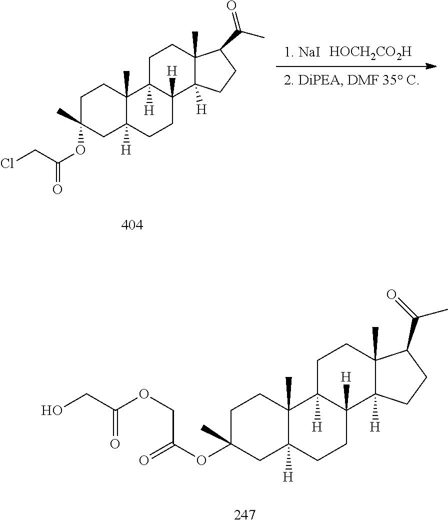

Following a similar procedure as in Example 85, but using glycolic acid (159 mg, 2.09 mmol) instead of 1,2-diamino-2-methylpropane, Compound 247 (170 mg) was obtained as a solid. ¹H NMR CDCl₃ 400 MHz δ: 4.74-4.60 (m, 2H), 4.30 (br. s. 2H), 2.53 (t, J=9.0 Hz, 1H), 2.32 (br. s. 1H), 2.28-2.09 (m, 5H), 2.06-1.98 (m, 1H), 1.94-1.86 (m, 1H), 1.76-1.09 (m, 18H), 1.04-0.82 (m, 1H), 0.78 (s, 3H), 0.61 (s, 3H).

### Example 90. Synthesis of Compound 142

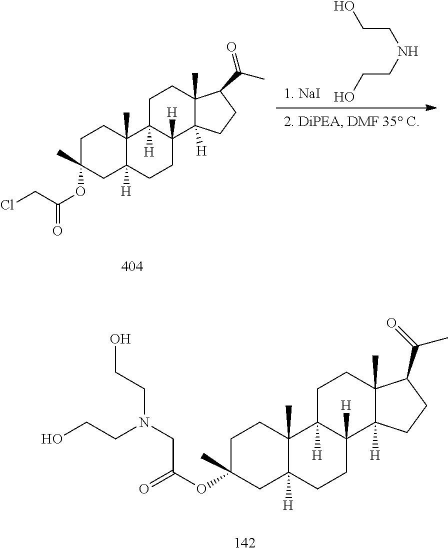

Following a similar procedure as in Example 85, but using diethanolamine (180 μL, 1.88 mmol) instead of 1,2-diamino-2-methylpropane, Compound 142 (108.6 mg) was obtained as an oil. HRMS (ESI+): calculated for C₂₈H₄₈NO₅ m/z [M+H]⁺: 478.3527, observed 478.3534; ¹H NMR δ: 3.56 (t, J=5.0 Hz, 6H), 3.41-3.31 (m, 2H), 2.86-2.75 (m, 4H), 2.54-2.43 (m, 1H), 2.25-2.06 (m, 5H), 2.00-1.92 (m, 1H), 1.71-1.08 (m, 18H), 0.97-0.80 (m, 2H), 0.72 (s, 3H), 0.71-0.62 (m, 1H), 0.55 (s, 3H).

### Example 91. Synthesis of Compound 234

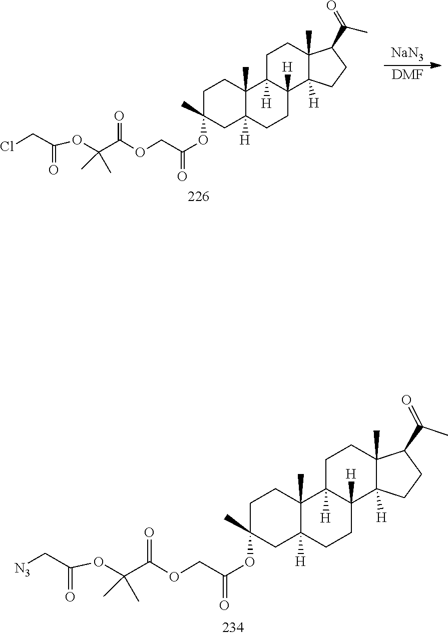

Compound 226 (from Example 82; 122 mg, 0.22 mmol) was added to DMF (4 mL) in an aluminum foil-covered vial, and sodium azide (66.4 mg, 1.02 mmol) was added in a single portion. The mixture was stirred at ambient temperature overnight, then diluted with ethyl acetate (50 mL), extracted with water (2×25 mL) then brine (25 mL), and the organic layer was dried with Na₂SO₄, filtered and concentrated to provide Compound 234 as a solid in 93% yield. ¹H NMR δ: 4.65-4.48 (m, 2H), 3.93-3.84 (m, 2H), 2.57-2.44 (m, 1H), 2.31-2.03 (m, 5H), 2.01-1.91 (m, 1H), 1.88-1.78 (m, 1H), 1.73-1.02 (m, 25H), 1.00-0.80 (m, 1H), 0.79-0.67 (m, 4H), 0.56 (s, 3H).

### Example 92. Synthesis of Compound 233

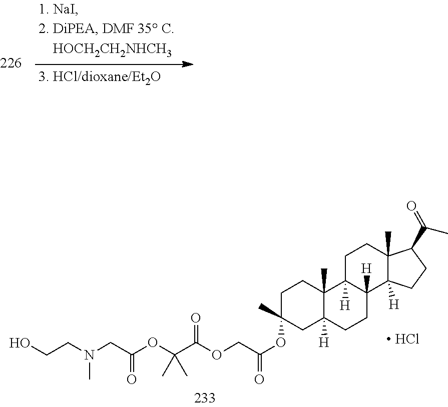

Compound 226 (from Example 82; 103 mg, 0.19 mmol), and sodium iodide (202 mg, 1.35 mmol) were dissolved in anhydrous DMF (4 mL) and stirred at ambient temperature for 15 minutes. Then DiPEA (450 μL, 2.58 mmol) was added in a single portion followed by 2-(methylamino)ethanol (80 μL, 1.07 mmol) and the resulting mixture was heated to 35° C. After stirring for 2.5 hours the reaction mixture was cooled and then diluted with ethyl acetate (40 mL), quenched with water (35 mL), extracted with water (2×25 mL) then brine (25 mL), and the organic layer was dried with Na₂SO₄ for 30 minutes, filtered and concentrated to provide a crude oil which was purified by column chromatography using an ISCO™ chromatography system to provide Compound 233 (free base; 58.8 mg) as an oil. ¹H NMR δ: 4.65-4.49 (m, 2H), 3.66-3.53 (m, 2H), 3.41-3.30 (m, 2H), 2.92-2.66 (m, 3H), 2.51 (t, J=8.8 Hz, 1H), 2.48-2.37 (m, 3H), 2.30-2.05 (m, 5H), 1.97 (d, J=11.7 Hz, 1H), 1.90-1.77 (m, 1H), 1.73-1.05 (m, 25H), 1.01-0.82 (m, 1H), 0.80-0.67 (m, 4H), 0.57 (s, 3).

The free base of Compound 233 (47.4 mg; 0.08 mmol) was then dissolved in a flask with 1,4-dioxane (2 mL), and a 1M solution of HCl in diethyl ether (90 μL, 0.09 mmol) was added in single portion, and the solution was stirred at ambient temperature for 15 minutes. The flask was then placed in dry ice for 30 minutes and frozen to a solid, and then lyophilized to provide Compound 233 as a solid hydrochloride salt. HRMS (ESI+): calculated for C₃₃H₅₄NO₈ m/z [M+H]⁺: 592.3844, observed 592.3851; ¹H NMR δ: 5.05 (q, J=5.1 Hz, 1H), 4.70-5.54 (m, 2H), 4.12 (br. s. 2H), 4.03 (br. s. 2H), 3.50 (br. s. 2H), 3.09 (s, 3H), 2.53 (t, J=9.1 Hz, 1H), 2.29-2.08 (m, 5H), 2.01 (d, J=11.7 Hz, 1H), 1.84 (d, J=8.8 Hz, 1H), 1.79-1.04 (m, 28H), 1.02-0.82 (m, 1H), 0.82-0.69 (m, 4H), 0.61 (s, 3H).

### Example 93. Synthesis of Compound 242

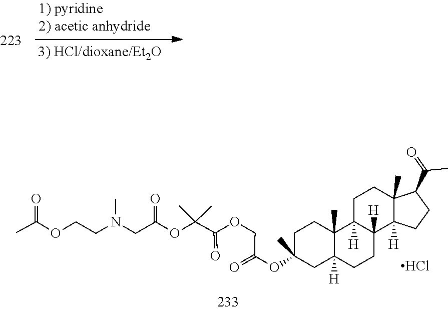

Compound 233 (from Example 92; 142 mg, 0.24 mmol) was dissolved in DCM (5 mL) and pyridine (250 μL) was added, and the mixture was then cooled to 0° C. in an ice water bath. After stirring for 5 minutes acetic anhydride (500 μL, 5.3 mmol) was added dropwise over −5 minutes. The resulting mixture was warmed to ambient temperature and stirred overnight. The reaction mixture was then concentrated under reduced pressure to provide an oil, which was then placed under high vacuum for a period of time to remove residual pyridine and acetic anhydride. The residue was purified by column chromatography using an ISCO™ chromatography system to provide Compound 242 (free base) as an oil in 68% yield. ¹H NMR δ: 4.63-4.46 (m, 2H), 4.20-4.10 (m, 2H), 3.40-3.32 (m, 2H), 2.82 (t, J=5.6 Hz, 2H), 2.50 (t, J=8.8 Hz, 1H), 2.45-2.40 (m, 3H), 2.30-2.16 (m, 1H), 2.16-2.03 (m, 6H), 2.02-1.93 (m, 1H), 1.88-1.76 (m, 1H), 1.71-1.53 (m, 8H), 1.52-1.04 (m, 18H), 1.01-0.81 (m, 1H), 0.80-0.67 (m, 4H), 0.57 (s, 3H).

The free base of Compound 242 (112.9 mg) was converted to the hydrochloride salt following the same procedure outlined in Example 92. HRMS (ESI+): calculated for C₃₅H₅₆NO₉ m/z [M+H]⁺: 634.3950, observed 634.3955; ¹H NMR δ: 5.04 (d, J=5.3 Hz, 1H), 4.61 (d, J=2.9 Hz, 2H), 4.54 (d, J=4.7 Hz, 2H), 3.98 (br. s. 2H), 3.55 (br. s. 2H), 3.00 (br. s. 3H), 2.53 (t, J=8.5 Hz, 1H), 2.29-2.09 9 m, 9H0, 2.0 (d, 10.5 Hz, 1H), 1.84 (d, J=11.1 Hz, 1H), 1.76-1.08 (m, 22H), 0.90 (br. S. 1H), 0.80-0.75 (m, 4H), 0.60 (s, 3).

### Example 94. Synthesis of Compound 238

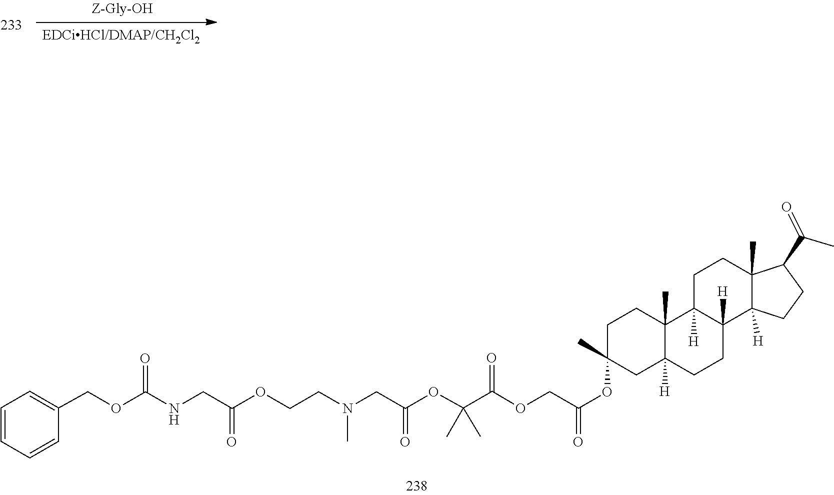

Compound 233 (from Example 92; 46.5 mg, 0.079 mmol), Z-Gly-OH (24.3 mg, 0.12 mmol) and DMAP (5.1 mg, 0.042 mmol) were stirred in DCM (3 mL). EDCi·HCl (24.4 mg, 0.13 mmol) was then added to the mixture to provide a solution that was stirred overnight at ambient temperature. The mixture was then diluted with water (10 mL) and stirred for 10 minutes, then transferred to a separatory funnel and the upper aqueous was discarded. The organic phase was then dried with Na₂SO₄, filtered and concentrated to provide a crude product which was purified by column chromatography using an ISCO™ chromatography system to provide Compound 238 (101.5 mg) as an oil. ¹H NMR δ: 7.44-7.22 (m, 5H), 5.70-5.65 (m, 1H), 5.18-5.02 (m, 2), 4.65-4.45 (m, 2H), 4.34-4.03 (m, 4H), 2.58-2.39 (m, 1H), 2.31-1.93 (m, 9H), 1.90-1.78 (m, 1H), 1.75-1.06 (m, 1H), 0.82-0.68 (m, 4), 0.58 (s, 3).

### Example 95. Synthesis of Compound 241

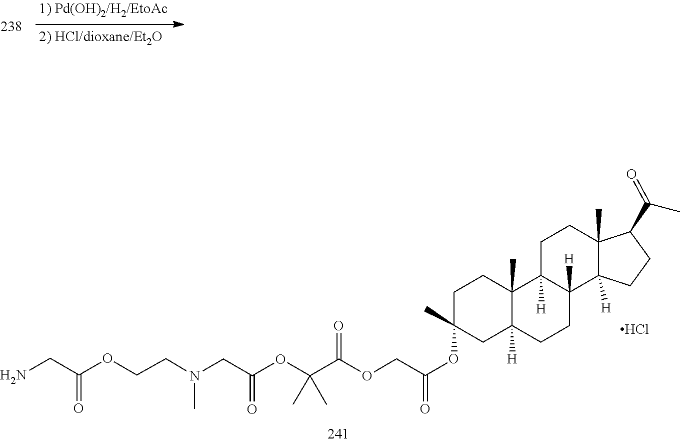

Compound 238 (from Example 94; 72 mg, 0.094 mmol) was dissolved in EtOAc (10 mL), and 20 wt % Pd(OH)₂ (17.8 mg, 0.025 mmol) was then added to the solution. The reaction was evacuated and back filled with hydrogen (H₂) gas three times, and then the mixture was stirred under an H₂ atmosphere for 22 hours. The reaction mixture was then filtered through a plug of Celite®, and the reaction vessel was rinsed with ethyl acetate (3×15 mL) and the washings were also passed through the Celite® plug. The filtrate was concentrated to a residue and purified by column chromatography using an ISCO™ chromatography system to provide Compound 241 (free base; 18.2 mg) as a solid. ¹H NMR δ: 4.67-4.48 (m, 2H), 4.32-4.20 (m, 2H), 3.51-3.43 (m, 1H), 3.43-3.30 (m, 2H), 2.86 (t, J=5.3 Hz, 2H), 2.74-2.60 (m, 1H), 2.58-2.49 (m, 1H), 2.49-2.40 (m, 3H), 2.26 (d, J=13.5 Hz, 1H), 2.16-2.09 (m, 3H), 2.07-1.95 (m, 2H), 1.75-1.06 (m, 26H), 1.03-0.82 (m, 2H), 0.81-0.70 (m, 4H), 0.60 (s, 3H).

The free base of Compound 241 was converted to the hydrochloride salt (solid; 12.7 mg) using the procedure in Example 92. HRMS (ESI+): calculated for C₃₅H₅₇N₂O₉ m/z [M+H]⁺: 649.4059, observed 649.4070; ¹H NMR CDCl3 400 MHz δ: 4.72-4.45 (m, 4H), 4.30-3.97 (m, 4H), 3.88-3.66 (m, 1H), 3.24-3.00 (m, 4H), 2.55 (t, J=8.8 Hz, 1H), 2.33-2.08 (m, 6H), 2.00 (d, J=12.1 Hz, 1H), 1.91-1.81 (m, 1H), 1.79-1.59 (m, 8H), 1.57-1.06 (m, 18H), 1.03-0.88 (m, 1H), 0.84-0.72 (m, 4H), 0.61 (s, 3H).

### Example 96. Synthesis of Compound 245

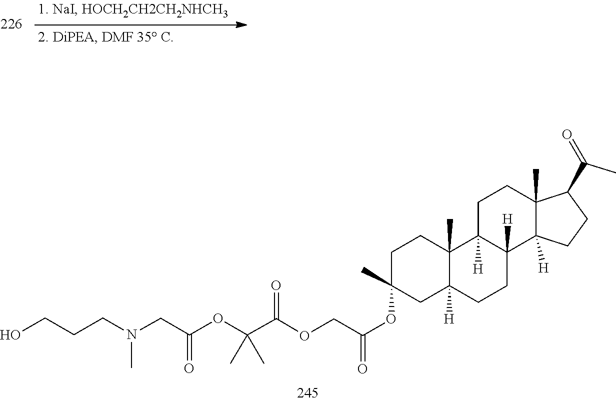

Following a similar procedure as in Example 92, but using 3-methylamino-1-propanol instead of 2-(methylamino)ethanol, Compound 245 was obtained as a foam in 78% yield. ¹H NMR CDCl₃ 400 MHz δ: 4.60-4.45 (m, 2H), 3.78-3.70 (m, 2H), 3.29-3.19 (m, 2H), 2.70-2.60 (m, 2H), 2.48 (t, J=9.0 Hz, 1H), 2.34 (s, 3H), 2.25-2.16 (m, 1H), 2.15-2.03 (m, 4H), 1.98-1.91 (m, 1H), 1.85-1.75 (m, 1H), 1.71-1.04 (m, 28H), 0.99-0.81 (m, 1H), 0.78-0.64 (m, 4H), 0.55 (s, 3H).

### Example 97. Synthesis of Compound 246

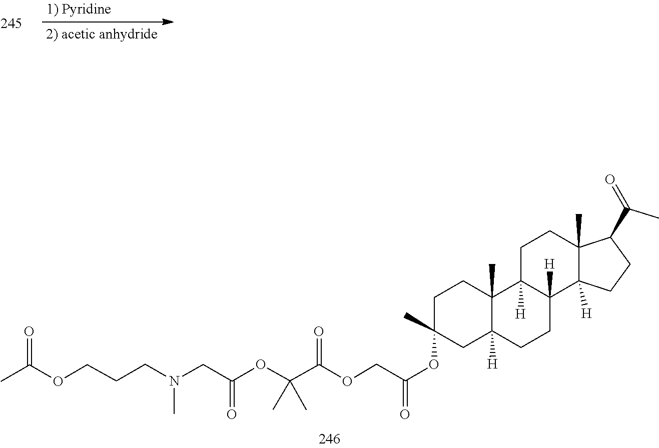

Following a similar procedure as in Example 93, but using Compound 245 (from Example 96) instead of Compound 233, Compound 246 was obtained as an oil. HRMS (ESI+): calculated for C₃₆H₅₈NO₉ m/z [M+H]⁺: 648.4106, observed 648.4123; ¹H NMR CDCl₃ 400 MHz δ: 4.61-4.45 (m, 2H), 4.14-4.02 (m, 2H), 3.31-3.20 (m, 2H), 2.62-2.53 (m, 2H), 2.49 (t, J=9.0 Hz, 1H), 2.35 (s, 3H), 2.22 (dd, J=2.7, 14.4 Hz, 1H), 2.16-2.04 (m, 4H), 2.02-1.91 (m, 4H), 1.85-1.72 (m, 3H), 1.70-1.52 (m, 9H), 1.51-1.04 (m, 16H), 0.98-0.84 (m 1H), 0.79-0.65 (m, 4H), 0.56 (s, 3H).

### Example 98. Synthesis of Compound 248

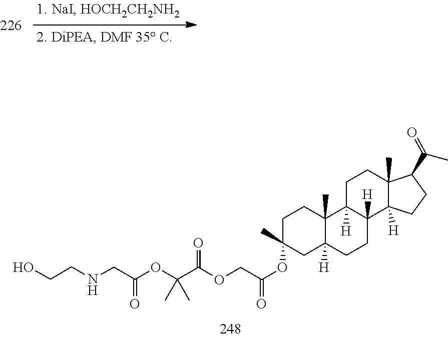

Following a similar procedure as in Example 92, but using aminoethanol instead of 2-(methylamino)ethanol, Compound 248 was obtained as an oil in 95% yield. ¹H NMR δ: 4.65-4.47 (m, 2H), 3.67-3.53 (m, 2H), 3.49-3.38 (m, 2H), 2.82-2.74 (m, 2H), 2.59-2.38 (m, 3H), 2.30-2.04 (m, 6H), 2.02-1.92 (m, 1H), 1.73-1.53 (m, 8H), 1.53-1.03 (m, 17H), 1.00-0.82 (m, 1H), 0.80-0.67 (m, 4H), 0.57 (s, 3H).

### Example 99. Synthesis of Compound 239

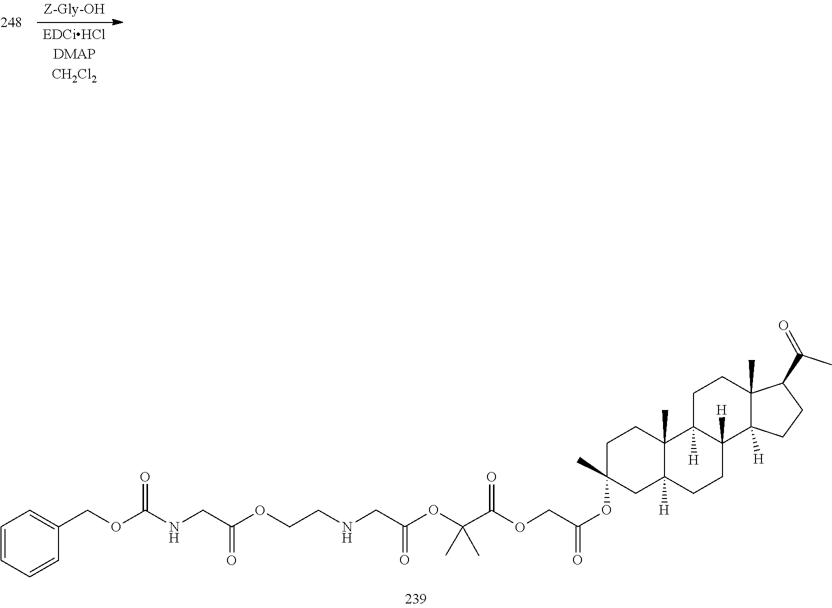

Following a similar procedure as in Example 94, but using Compound 248 instead of Compound 233, Compound 239 was obtained as a solid in 39% yield. ¹H NMR δ: 7.44-7.26 (m, 5H), 5.43 (br. s. 1H), 5.18-5.08 (m, 2H), 4.65-4.48 (m, 2H), 4.25 (t, J=5.3 Hz, 2H), 4.00 (d, J=5.3 Hz, 2H), 3.36 (s, 2H), 2.84 (t, J=5.6 Hz, 2H), 2.57-2.36 (m, 4H), 2.31-2.06 (m, 5H), 2.04-1.94 (m, 1H), 1.90-1.78 (m, 1H), 1.74-1.04 (m, 24H), 1.02-0.68 (m, 6H), 0.59 (s, 3).

### Example 100. Synthesis of Compound 235

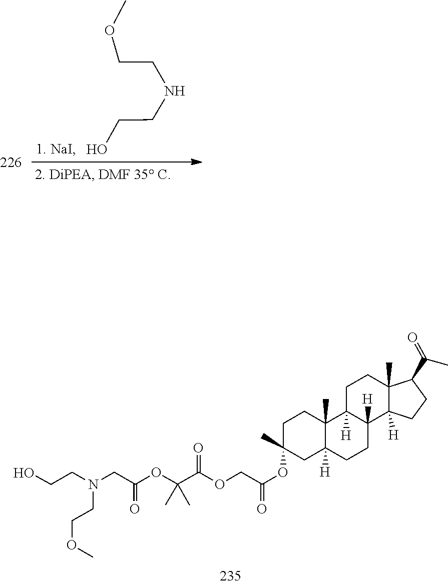

Following a similar procedure as in Example 92, but using 2-[(2-methoxyethyl)amino]ethan-1-ol instead of 2-(methylamino)ethanol, Compound 235 was obtained as an oil in 97% yield. ¹H NMR δ: 4.66-4.48 (m, 2H), 3.58-3.49 (m, 4H), 3.49-3.42 (m, 2H), 3.34 (s, 3H), 2.98-2.81 (m, 8H), 2.52 (t, J=9.1 Hz, 1H), 2.31-2.07 (m, 5H), 1.99 (d, J=11.7 Hz, 1H), 1.89-1.80 (m, 1H), 1.73-1.07 (m, 22H), 0.99-0.86 (m, 1H), 0.81-0.69 (m, 4H), 0.59 (s, 3H).

### Example 101. Synthesis of Compound 243

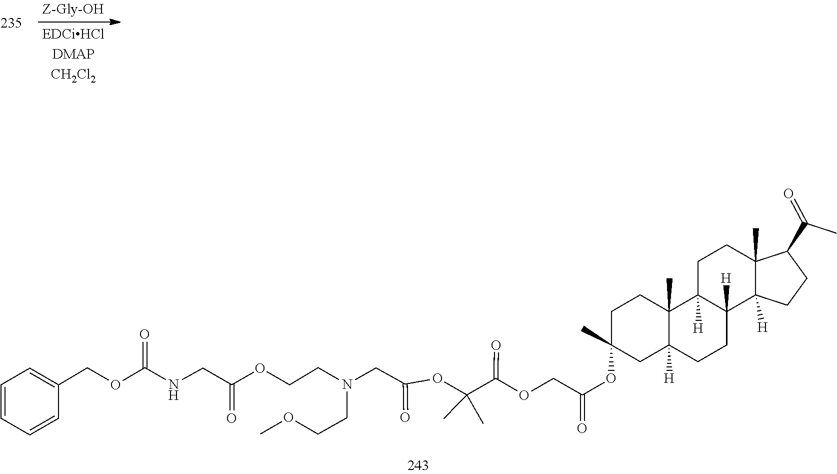

Following a similar procedure as in Example 94, but using Compound 235 (from Example 100) instead of Compound 233, Compound 243 was obtained as a solid in 88% yield. ¹H NMR δ: 7.44-7.29 (m, 5H), 5.38 (br. s. 1H), 5.14 (s, 2H), 4.66-4.50 (m, 2H), 4.24 (t, J=5.6 Hz, 2H), 4.01 (d, J=5.3 Hz, 2H), 3.59-3.40 (m, 4H), 3.33 (s, 3H), 3.04-2.85 (m, 4H), 2.60-2.48 (m, 1H), 2.32-2.08 (m, 5H), 2.08-1.96 (m, 1H), 1.85 (d, J=10.5 Hz, 1H), 1.76-1.09 (m, 25H), 1.05-0.85 (m, 1H), 0.80-0.76 (m, 4H), 0.61 (s, 3H).

### Example 102. Synthesis of Compound 236

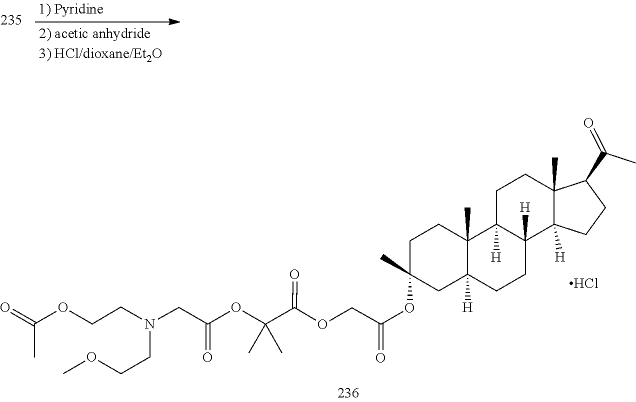

Following a similar procedure as in Example 93, but using Compound 235 (from Example 100) instead of Compound 233, Compound 236 (free base) was obtained as an oil. ¹H NMR δ: 4.65-4.48 (m, 2H), 4.13 (t, J=6.2 Hz, 2H), 3.61-3.52 (m, 2H), 3.46 (t, J=5.6 Hz, 2H), 2.94 (td, J=5.8, 16.4 Hz, 4H), 2.52 (t, J=8.8 Hz, 1H), 2.34-1.94 (m, 1OH), 1.90-1.79 (m, 1H), 1.76-1.05 (m, 24H), 1.02-0.83 (m, 1H), 0.83-0.69 (m, 4H), 0.59 (s, 3H). The free base of Compound 236 was converted to the hydrochloride salt (solid; 79% yield) following the procedure used in Example 92. HRMS (ESI+): calculated for C₃₇H₅₉NO₁₀ m/z [M+H]⁺: 678.4212, observed 678.4208; ¹H NMR CDCl3 400 MHz δ: 4.67-4.51 (m, 2H), 4.44 (t, J=5.1 Hz, 2H), 4.05-3.91 (m, 2H), 3.79 (d, J=4.3 Hz, 2H), 3.55-3.26 (m, 4H), 2.57-2.47 (m, 1H), 2.31-2.06 (m, 9H), 2.03-1.94 (m, 1H), 1.90-1.80 (m, 1H), 1.76-1.57 (m, 9H), 1.54-1.05 (m, 19H), 1.00-0.86 (m, 1H), 0.83-0.71 (m, 4H), 0.59 (s, 3H).

### Example 103. Synthesis of Compound 240

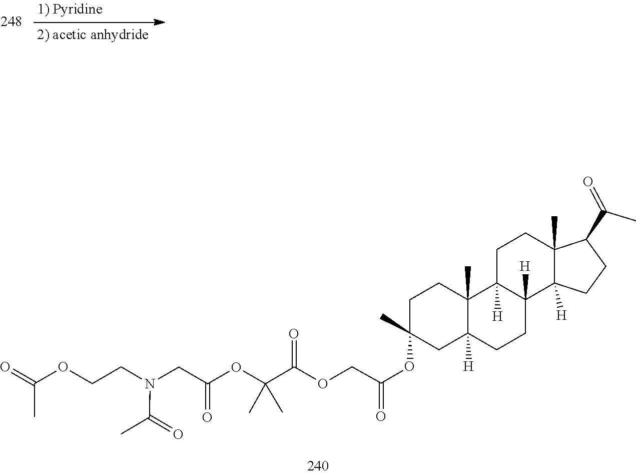

Following a similar procedure as in Example 93, but using Compound 248 (from Example 98) instead of Compound 233, Compound 240 (75.4 mg) was obtained. HRMS (ESI+): calculated for C₃₆H₅₆NO₁₀ m/z [M+H]⁺: 662.3899, observed 662.3910; ¹H NMR δ: 4.63-4.46 (m, 2H), 4.25-4.03 (m, 5H), 3.62 (q, J=5.7 Hz, 2H), 2.50 (t, J=8.8 Hz, 1H), 2.30-2.03 (m, 11H), 2.03-1.90 (m, 1H), 1.91-1.77 (m, 2H), 1.77-1.06 (m, 24H), 1.02-0.72 (m, 5H), 0.57 (s, 3).

### Example 104. Synthesis of Compound 139

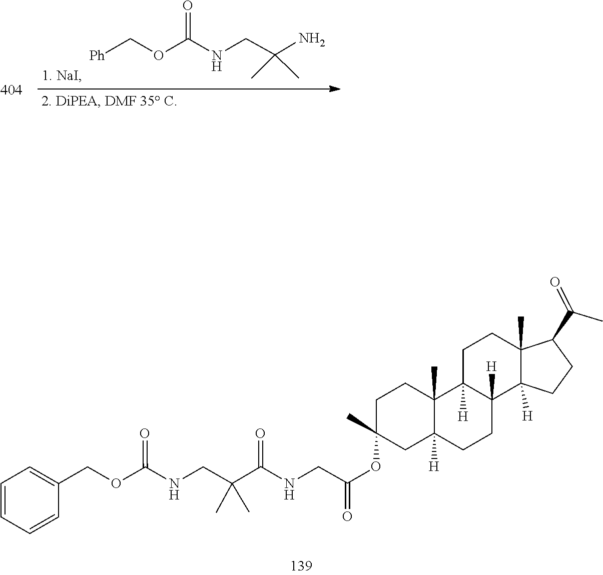

Following a similar procedure as in Example 85, but using benzyl (2-amino-2-methylpropyl)carbamate instead of 1,2-diamino-2-methylpropane, Compound 139 was obtained as an oil in 71% yield. HRMS (ESI+): calculated for C₃₆H₅₅N₂O₅ m/z [M+H]⁺: 595.4105, observed 595.4121; ¹H NMR δ: 7.40-7.19 (m, 5H), 5.50 (t, J=5.6 Hz, 1H), 5.16-4.97 (m, 2H), 3.25 (s, 2H), 3.04 (d, J=5.9 Hz, 2H), 2.57-2.39 (m, 1H), 2.24-1.80 (m, 6H), 1.73-0.80 (m, 28H), 0.77-0.64 (m, 4H), 0.55 (s, 3H).

### Example 105. Synthesis of Compound 231

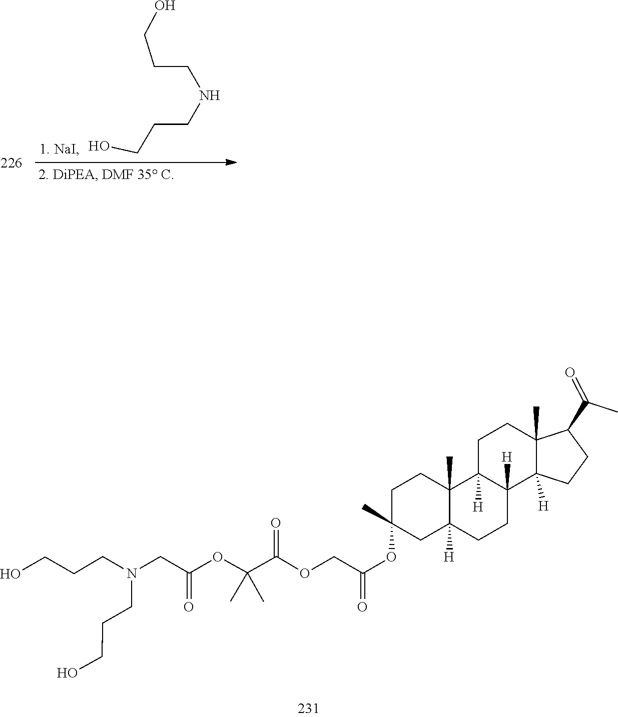

Following a similar procedure as in Example 92, but using 1,2-diamino-2-methylpropane instead of 2-(methylamino)ethanol, Compound 231 was obtained as an oil in 41% yield. ¹H NMR δ: 3.42-3.30 (m, 2H), 3.12 (br. s. 4H), 2.62-2.42 (m, 3H), 2.26-2.03 (m, 5H), 2.02-1.82 (m, 2H), 1.71-1.00 (m, 22H), 0.98-0.80 (m, 2H), 0.78-0.66 (m, 5H), 0.61-0.51 (s, 3H).

### Example 106. Synthesis of Compound 237

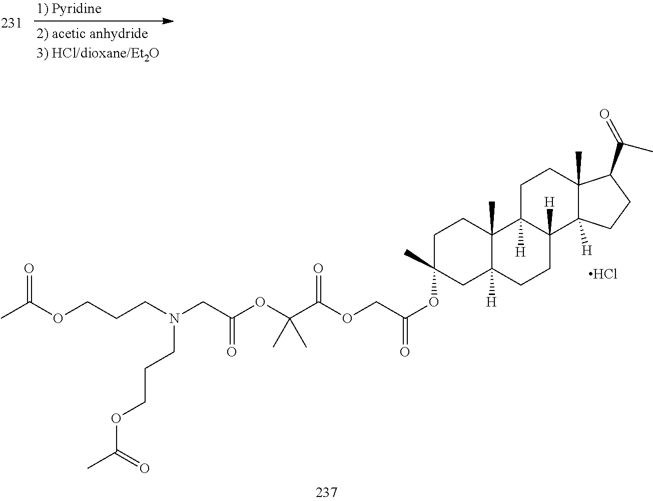

Following a similar procedure as in Example 93, but using Compound 231 (from Example 105) instead of Compound 233, Compound 237 (free base) was obtained as an oil in 91% yield. ¹H NMR δ: 4.65-4.50 (m, 2H), 4.12 (t, J=6.4 Hz, 4H), 3.37 (s, 2H), 2.70 (t, J=7.0 Hz, 4H), 2.54 (t, J=8.8 Hz, 1H), 2.34-1.98 (m, 13H), 1.92-1.09 (m, 29H), 1.05-0.87 (m, 1H), 0.83-0.71 (m, 4H), 0.61 (s, 3H). The free base of Compound 237 was converted to the hydrochloride salt (solid; 98% yield) using the procedure in Example 92. HRMS (ESI+): calculated for C₄₀H₆₄NO₁₁ m/z [M+H]⁺: 734.4474, observed 734.4452; ¹H NMR δ: 4.70-4.53 (m, 2H), 4.17 (t, J=5.9 Hz, 4H), 3.92 (br. s. 2H), 3.35 (br. s. 4H), 2.58-2.48 (m, 1H), 2.37-2.15 (m, 4H), 2.12 (s, 3H), 2.08-1.97 (m, 8H), 1.85 (d, J=8.8 Hz, 1H), 1.78-1.07 (m, 24H), 1.03-0.82 (m, 1H), 0.78-0.72 (m, 4H), 0.61 (s, 3H).

### Example 107. Synthesis of Compounds 229 and 230

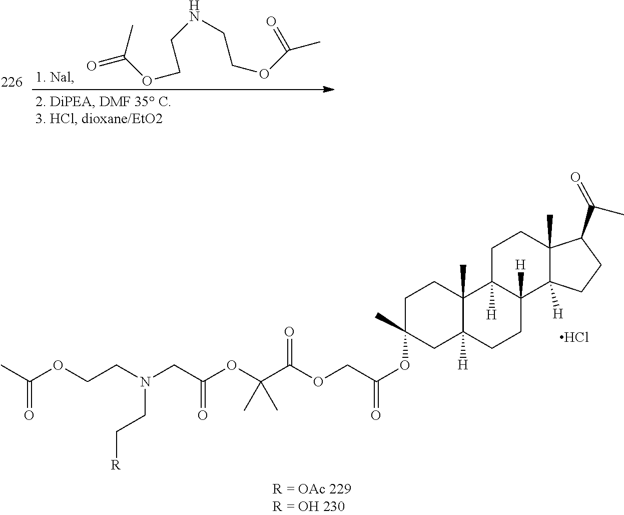

Following a similar procedure as in Example 92, but using 2-[(2-acetoxyethy)amino]ethyl acetate HCl instead of 2-(methylamino)ethanol, a mixture of Compound 229 (free base) and Compound 230 (free base) was obtained. The mixture was purified by ISCO chromatography (gradient 0% EtOAc for 2 minutes, then ramping to 30% over 12 minutes, then holding at 30% until the product eluted, then ramping to 100% EtOAc) to provide:

Compound 229 (free base) as an oil in 31% yield. ¹H NMR δ: 4.62-4.54 (m, 2H), 4.12 (t, J=5.9 Hz, 4H), 3.56-3.50 (m, 2H), 2.98 (t, J=5.9 Hz, 4H), 2.52 (t, J=8.8 Hz, 1H), 2.32-2.13 (m, 2H), 2.11 (s, 3H), 2.08-2.04 (m, 5H), 1.99 (d, J=11.7 Hz, 1H), 1.88-1.80 (m, 1H), 1.76-1.54 (m, 1OH), 1.54-1.06 (m, 17H), 1.01-0.84 (m, 1H), 0.81-0.68 (m, 4H), 0.59 (s, 3H); and

Compound 230 (free base) as an oil in 16% yield. ¹H NMR δ: 4.66-4.50 (m, 2H), 4.20-4.11 (m, 2H), 3.59-3.47 (m, 4H), 2.98 (t, J=5.6 Hz, 2H), 2.87 (t, J=5.0 Hz, 2H), 2.52 (t, J=8.8 Hz, 1H), 2.31-2.09 (m, 5H), 2.07 (s, 3H), 2.04-1.95 (m, 1H), 1.89-1.79 (m, 1H), 1.73-1.05 (m, 26H), 1.00-0.84 (m, 1H), 0.81-0.70 (m, 4H), 0.59 (s, 3H).

The free base of Compound 229 was converted to the hydrochloride salt (solid; 83% yield) using the procedure in Example 92. HRMS (ESI+): calculated for C₃₈H₆₀NO₁₁ m/z [M+H]⁺: 706.4161, observed 706.4151; ¹H NMR δ: 4.69-4.54 (m, 2H), 4.43 (br. s. 4H), 3.91 (br. s. 2H), 3.38 (br. s. 4H), 2.53 (t, J=8.8 Hz, 1H), 2.31-2.14 (m, 2H), 2.12-2.09 (m, 8H), 2.07 (d, J=2.9 Hz, 1H), 2.03 (br. s. 1H), 1.90-1.86 (m, 1H), 1.83 (br. s. 1H), 1.77-1.63 (m, 1OH), 1.63-1.46 (m, 8H), 1.45-1.09 (m, 1OH), 1.01-0.84 (m, 1H), 0.80-0.76 (m, 4H), 0.61 (s, 3H).

The free base of Compound 230 was converted to the hydrochloride salt (solid; 97% yield) using the procedure in Example 92. HRMS (ESI+): calculated for C₃₆H₅₈NO₁₀ m/z [M+H]⁺: 664.4055, observed 664.4072; ¹H NMR δ: 4.62 (d, J=2.9 Hz, 2H), 4.54 (d, J=4.1 Hz, 2H), 4.13 (s, 2H), 3.97 (d, J=5.3 Hz, 2H), 3.59 (d, J=4.1 Hz, 2H), 3.44 (d, J=5.9 Hz, 2H), 2.53 (t, J-9.1 Hz, 1H), 2.28-2.17 (m, 1H), 2.15-2.12 (m, 6H), 2.06-1.97 (m, 2H), 1.83 (br. s. 1H), 1.80-1.65 (m, 8H), 1.54-1.08 (m, 18H), 1.01-0.84 (m, 1H), 0.82-0.72 (m, 4H), 0.61 (S, 3H).

### Example 108. Synthesis of Compound 141

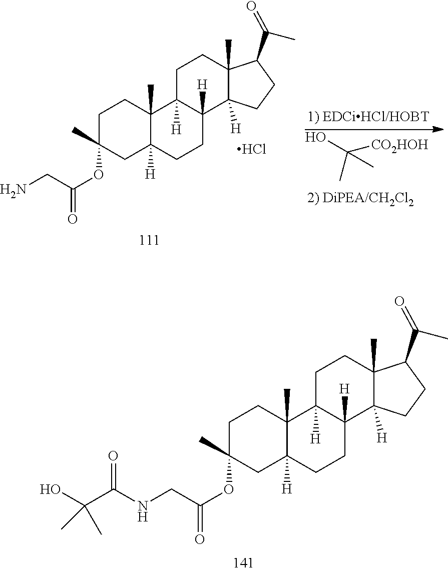

A mixture of Compound 111 (HCl salt, 81.4 mg, 0.19 mmol) and 2-hydroxyisobutyric acid (74.7 mg, 0.72 mmol) was stirred in DCM (4 mL) at 0° C., then EDCi·HCl (79.4 mg, 0.41 mmol) and then HOBt (41.4 mg, 0.31 mmol) were added, followed by DiPEA (150 μL, 0.86 mmol). The resulting pale-yellow solution was slowly warmed to ambient temperature overnight, then diluted with EtOAc (25 mL) and quenched with a 1N solution of HCl (25 mL). The organic phase was washed with saturated NaHCO₃ (25 mL) followed by saturated NaCl (25 mL), and then dried with Na₂SO₄, filtered and concentrated. The crude product was purified by column chromatography using an ISCO™ chromatography system to provide Compound 141 (19.5 mg) as a solid. HRMS (ESI+): calculated for C₂₈H₄₆NO₅ m/z [M+H]⁺: 476.3370, observed 476.3373; ¹H NMR δ: 7.18 (br. s. 1H), 4.03-3.95 (m, 2H), 2.68 (br. s. 1H), 2.53 (t, J=9.1 Hz, 1H), 2.29-2.06 (m, 7H), 1.99 (d, J=11.7 Hz, 1H), 1.92-1.07 (m, 21H), 1.03-0.83 (m, 3H), 0.83-0.69 (m, 5H), 0.60 (s, 3H).

### Example 109. Synthesis of Compound 143

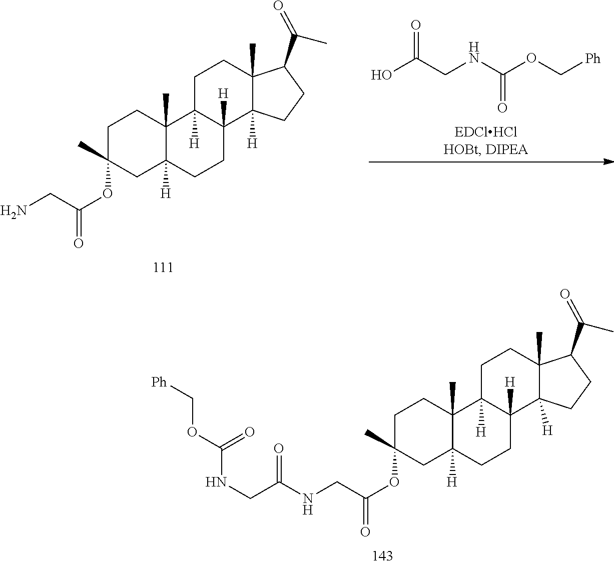

Following a similar procedure as in Example 35, but using Z-Gly-OH instead of 4-morpholinylacetic acid, Compound 143 will be obtained.

### Example 110. Synthesis of Compound 150

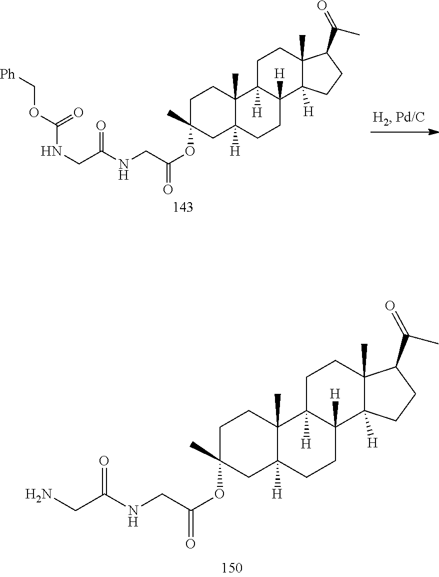

Following a similar procedure as in Example 81, but using Compound 143 (from Example 109) instead of Compound 128, Compound 150 will be obtained.

### Example 111. Synthesis of Compound 144

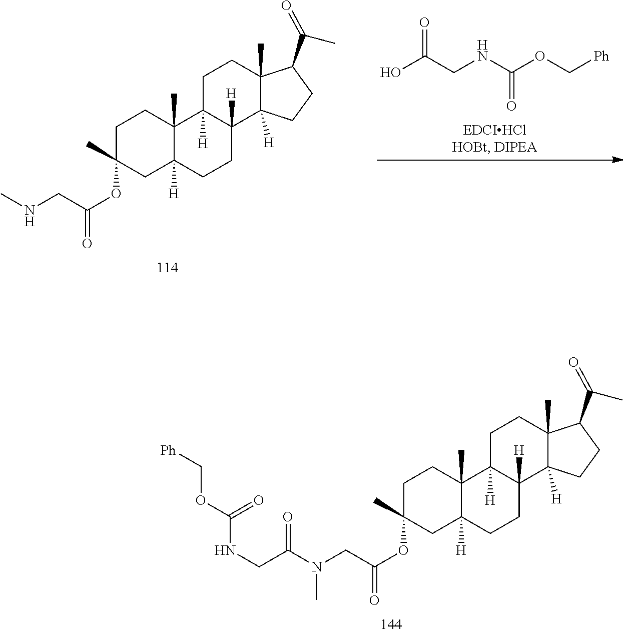

Compound 114 (From Example 36; 133.8 mg, 0.33 mmol), Z-Gly-OH (90.8 mg, 0.43 mmol) and DMAP (10.1 mg, 0.083 mmol) were dissolved in DCM (10 mL), and the mixture was cooled to 0° C. EDCi·HCl (98.7 mg, 0.51 mmol) was then added in a single portion, and the resulting solution was warmed to ambient temperature overnight. The mixture was then diluted with EtOAc (40 mL) and quenched with water (35 mL). The aqueous phase was separated, and the organic phase was washed with water (2×25 mL) then brine (25 mL), dried with Na₂SO₄ for 30 minutes, filtered and concentrated to provide an oil, which was purified by column chromatography using an ISCO™ chromatography system to provide Compound 144 (133.2 mg) as an oil. LCMS (ESI+): Rf=7.57, (M+H)=595.41; ¹H NMR 400 MHz δ: 7.42-7.29 (m, 5H), 5.79 (br. s., 1H), 5.18-5.10 (m, 2H), 4.16-3.99 (m, 4H), 3.10-3.30 (s, 22.51-2.44 (m, 1H), 2.28-2.20 (m, 1H), 2.20-2.07 (m, 4H), 1.99 (d, J=12.1 Hz, 1H), 1.74-1.04 (m, 19H), 1.00-0.87 (m, 1H), 0.81-0.69 (m, 4H), 0.59 (s, 3H).

### Example 112. Synthesis of Compound 149

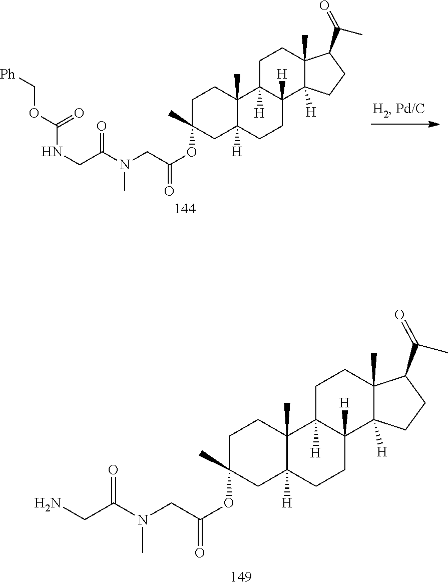

Following a similar procedure as in Example 81, but using Compound 144 (from Example 111) instead of Compound 128, Compound 149 will be obtained.

### Example 113. Synthesis of Compound 145

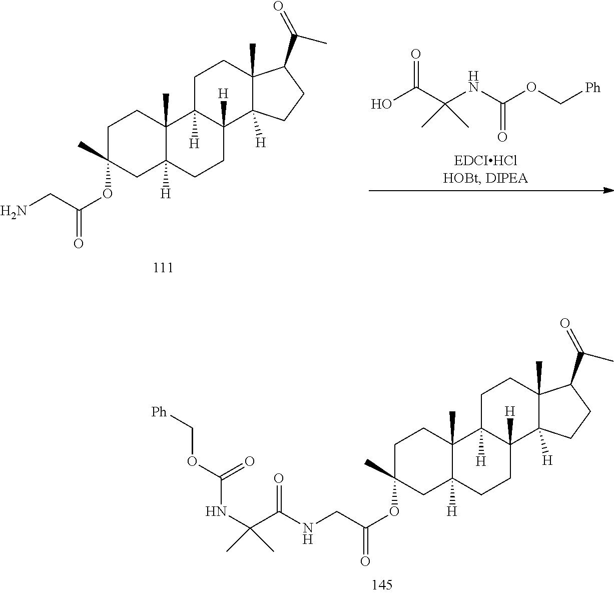

Following a similar procedure as in Example 35, but using Z-Aib-OH instead of 4-morpholinylacetic acid, Compound 144 will be obtained.

### Example 114. Synthesis of Compound 152

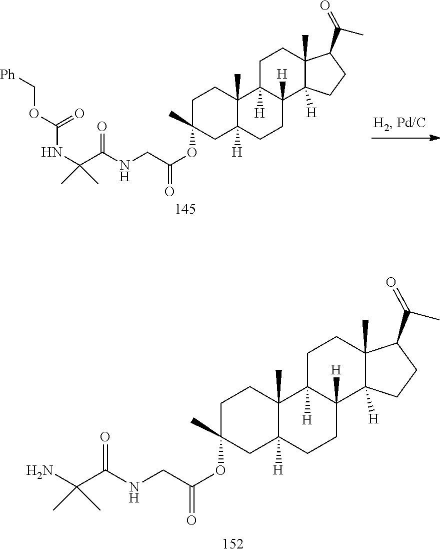

Following a similar procedure as in Example 81, but using Compound 145 (from Example 113) instead of Compound 128, Compound 152 will be obtained.

### Example 115. Synthesis of Compound 146

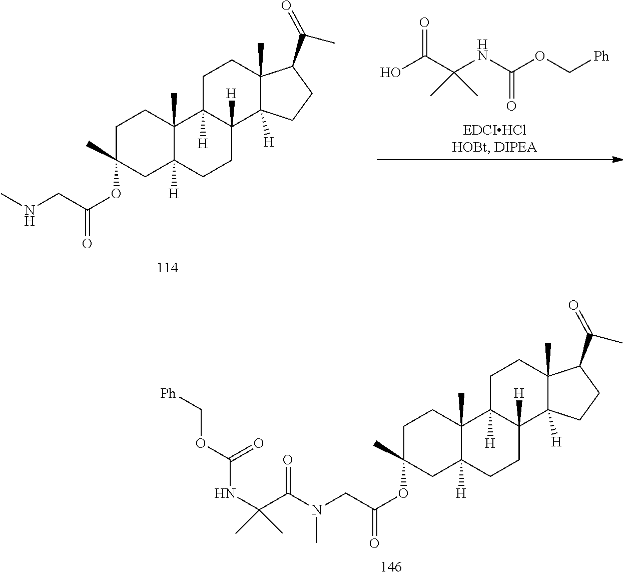

Following a similar procedure as in Example 35, but using Z-Aib-OH instead of 4-morpholinylacetic acid, and Compound 114 instead of Compound 111, Compound 146 will be obtained.

### Example 116. Synthesis of Compound 151

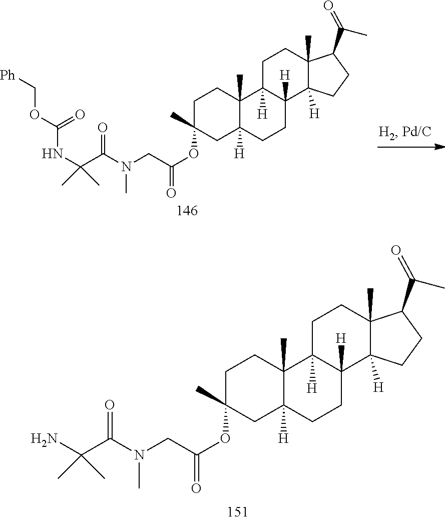

Following a similar procedure as in Example 81, but using Compound 146 (from Example 115) instead of Compound 128, Compound 151 will be obtained.

### Example 117. Synthesis of Compound 147

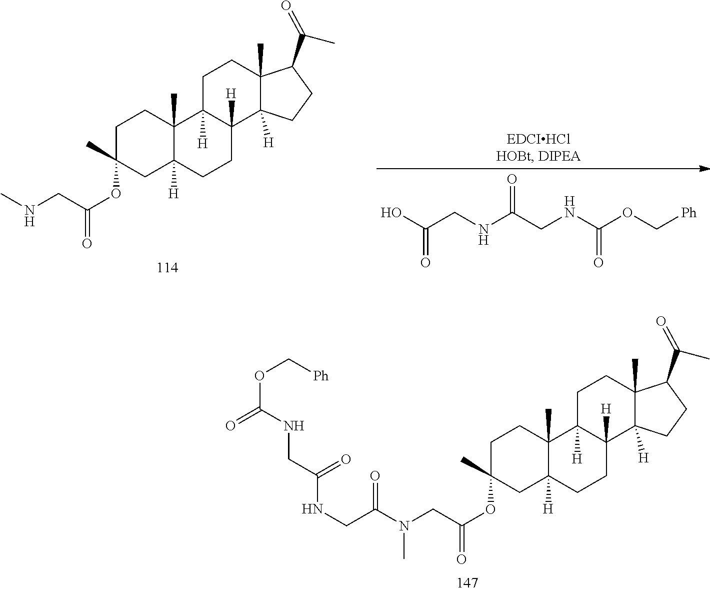

Following a similar procedure as in Example 35, but using (2-benzyloxycarbonylamino-acetylamino)-acetic acid instead of 4-morpholinylacetic acid, and Compound 114 instead of Compound 111, Compound 147 will be obtained.

### Example 118. Synthesis of Compound 154

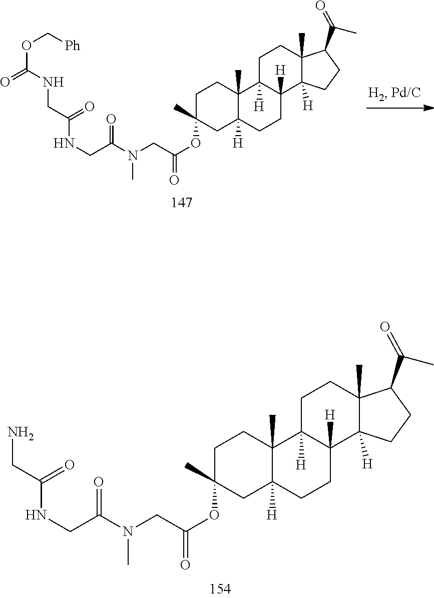

Following a similar procedure as in Example 81, but using Compound 147 (from Example 117) instead of Compound 128, Compound 154 will be obtained.

### Example 119. Physicochemical Properties of Compounds

The physicochemical properties of the compounds (Log S, Log D, Log P, TPSA, acid pKa and base pKa) disclosed herein were calculated using commercially available software (ChemAxon), and provided below in Table 2.1.

TABLE 2.1
Calculated physicochemical properties of exemplary compounds.

| Compound | | Log S | Log S | Log D | | | acid | base |
|---|---|---|---|---|---|---|---|---|
| No. | Salt | pH 7.4 | pH 2.0 | pH 7.4 | LogP | TPSA | pKa | pKa |
| ganaxolone | — | −5.38 | −5.38 | 4.27 | 4.27 | 37 | 19.1 | −1.0 |
| 100 | HCl | NC | NC | 6.18 | 6.18 | 59 | 19.4 | 5.4 |
| 101 | HCl | NC | NC | 4.38 | 4.38 | 56 | 19.4 | 4.6 |
| 102 | HCl | NC | NC | 4.15 | 4.45 | 50 | 19.4 | 7.4 |
| 103 | HCl | NC | NC | 4.20 | 4.20 | 82 | 19.4 | 2.7 |
| 104 | HCl | NC | NC | 4.52 | 4.55 | 56 | 19.4 | 6.3 |
| 105 | HCl | NC | NC | 4.58 | 4.58 | 73 | 19.4 | 4.1 |
| 106 | HCl | NC | NC | 4.41 | 4.41 | 82 | 19.4 | 2.8 |
| 107 | HCl | NC | NC | 4.89 | 3.53 | 76 | 15.6 | 6.0 |
| 108 | HCl | NC | NC | 5.30 | 5.30 | 82 | 19.4 | 3.1 |
| 109 | — | NC | NC | 4.01 | 4.89 | 82 | 19.4 | 2.7 |
| 110 | HCl | NC | NC | 3.84 | 3.84 | 82 | 19.4 | 2.7 |
| 111 | HCl | −5.61 | −0.70 | 3.61 | 3.78 | 69 | 19.4 | 7.1 |
| 112 | HCl | −5.61 | NC | 3.27 | 3.28 | 85 | 12.62 | 5.2 |
| 113 | — | NC | NC | 4.27 | 4.28 | 85 | 12.6 | 5.8 |
| 114 | HCl | −5.54 | NC | 4.17 | 4.22 | 55 | 19.4 | 6.4 |
| 115 | HCl | −5.41 | NC | 3.50 | 3.50 | 76 | 19.4 | 5.4 |
| 116 | — | −4.60 | NC | 2.37 | 3.51 | 88 | 14.9 | 8.5 |
| 117 | — | NC | NC | 5.19 | 5.19 | 140 | 12.2 | −4.3 |
| 118 | — | NC | NC | 6.30 | 6.30 | 111 | 12.5 | −4.6 |
| 119 | — | NC | NC | 7.40 | 7.40 | 82 | 13.6 | −7.0 |
| 120 | — | −4.04 | −5.01 | 1.60 | 3.39 | 93 | 3.1 | 5.3 |
| 121 | — | NC | NC | 6.52 | 6.52 | 82 | 13.5 | −7.0 |
| 122 | HCl | −6.20 | NC | 2.16 | 3.03 | 128 | 12.4 | 8.2 |
| 124 | — | NC | NC | 0.56 | 2.50 | 93 | 3.0 | 5.2 |
| 125 | — | NC | NC | 4.84 | 4.84 | 82 | 19.4 | 2.9 |
| 126 | — | NC | NC | 5.90 | 5.90 | 82 | 19.4 | 2.9 |
| 127 | — | NC | NC | 6.57 | 6.57 | 82 | 19.4 | 2.9 |
| 128 | — | NC | NC | 6.57 | 6.57 | 73 | 19.4 | 4.7 |
| 129 | — | NC | NC | 6.13 | 6.13 | 82 | 19.4 | 2.8 |
| 130 | | NC | NC | 4.41 | 4.41 | 82 | 19.4 | 2.8 |
| 131 | — | −6.88 | NC | 1.15 | 3.02 | 93 | 3.0 | 5.3 |
| 132 | — | −5.97 | −1.67 | 4.20 | 4.22 | 76 | 14.8 | 6.1 |
| 133 | | NC | NC | 3.87 | 3.91 | 67 | 15.6 | 6.3 |
| 134 | | NC | NC | 4.66 | 4.67 | 82 | 19.4 | 5.68 |
| 135 | — | −5.63 | −3.21 | 5.90 | 5.91 | 82 | 19.4 | 5.7 |
| 136 | — | −5.63 | NC | 4.33 | 4.34 | 94 | 19.4 | 5.7 |
| 137 | — | NC | NC | 1.59 | 2.85 | 84 | 3.1 | 5.9 |
| 138 | — | −3.46 | 0.00 | 1.77 | 4.12 | 81 | 19.4 | 9.8 |
| 139 | | NC | NC | 6.27 | 6.28 | 94 | 14.8 | 5.9 |
| 140 | — | −5.78 | −1.53 | 4.19 | 4.22 | 76 | 15.0 | 6.3 |
| 141 | — | −6.27 | −6.27 | 3.79 | 3.79 | 93 | 12.4 | −3.5 |
| 142 | — | −5.02 | −0.91 | 3.20 | 3.22 | 87 | 15.3 | 6.1 |
| 143 | — | −7.66 | −7.66 | 4.84 | 4.84 | 111 | 12.4 | −5.2 |
| 144 | — | −7.44 | −7.44 | 5.07 | 5.07 | 102 | 13.5 | −5.0 |
| 145 | — | −8.02 | −8.02 | 5.84 | 5.84 | 111 | 12.4 | −4.7 |
| 146 | — | −7.80 | −7.80 | 6.07 | 6.07 | 102 | 13.4 | −4.5 |
| 147 | — | −7.42 | −7.42 | 3.96 | 3.96 | 131 | 12.5 | −4.5 |
| 148 | — | −7.65 | −7.65 | 3.74 | 3.74 | 140 | 12.1 | −4.6 |
| 149 | — | −5.76 | 0.00 | 2.10 | 2.90 | 90 | 19.4 | 8.1 |
| 150 | — | −5.95 | 0.00 | 1.87 | 2.68 | 98 | 12.7 | 8.1 |
| 151 | — | −6.18 | 0.00 | 2.65 | 3.90 | 90 | 19.4 | 8.6 |
| 152 | — | −6.37 | 0.00 | 2.42 | 3.68 | 98 | 12.7 | 8.6 |
| 153 | — | −6.06 | −0.22 | 1.00 | 1.57 | 128 | 12.3 | 7.8 |
| 154 | — | −5.86 | −0.02 | 1.22 | 1.80 | 119 | 12.7 | 7.8 |
| 200 | HCl | −5.53 | −3.17 | 3.89 | 4.01 | 82 | 19.4 | 4.4 |
| 201 | — | −5.69 | −5.69 | 3.89 | 3.89 | 64 | 13.1 | −3.6 |
| 202 | HCl | −5.64 | NC | 4.16 | 4.22 | 73 | 19.4 | 6.6 |
| 203 | — | NC | NC | 4.75 | 4.75 | 79 | 19.4 | −4.1 |
| 204 | — | NC | NC | 4.11 | 4.11 | 88 | 19.4 | −3.7 |
| 205 | — | −5.54 | −5.54 | 4.06 | 4.06 | 97 | 19.4 | −3.7 |
| 206 | — | NC | NC | 8.11 | 8.11 | 137 | 12.2 | −3.7 |
| 207 | — | NC | NC | 5.03 | 5.03 | 137 | 12.1 | −4.9 |
| 208 | bis | NC | NC | 3.78 | 4.07 | 76 | 19.4 | 7.4 |
| | HCl | | | | | | | |
| 209 | — | NC | NC | 5.92 | 5.92 | 137 | 12.2 | −4.6 |
| 210 | — | NC | NC | 6.41 | 6.41 | 99 | 19.4 | −6.9 |
| 211 | HCl | −5.85 | NC | 4.57 | 4.57 | 82 | 19.4 | 4.6 |
| 212 | HCl | −5.85 | NC | 4.57 | 4.57 | 82 | 19.4 | 4.6 |
| 213 | HCl | −6.09 | NC | 5.01 | 5.01 | 82 | 19.4 | 4.9 |
| 214 | HCl | −5.86 | NC | 2.62 | 3.76 | 125 | 12.4 | 8.5 |
| 215 | HCl | −7.75 | NC | 4.88 | 5.95 | 125 | 12.4 | 8.4 |
| 216 | HCl | −5.25 | NC | 1.85 | 2.87 | 125 | 12.3 | 8.4 |
| 217 | HCl | NC | NC | 3.10 | 4.24 | 85 | 14.8 | 8.5 |
| 218 | — | −5.89 | −0.71 | 4.00 | 4.41 | 96 | 19.4 | 7.6 |
| 219 | — | NC | NC | 5.55 | 5.56 | 70 | 9.1 | −6.8 |
| 220 | HCl | NC | NC | 3.92 | 4.97 | 82 | 19.4 | 8.4 |
| 221 | — | NC | NC | 5.76 | 5.76 | 108 | 19.4 | −5.6 |
| 222 | TFA | NC | NC | 3.48 | 4.48 | 91 | 19.4 | 8.4 |
| 223 | — | NC | NC | 6.57 | 6.57 | 108 | 13.2 | −6.9 |
| 224 | — | −6.2 | −6.2 | 4.52 | 4.52 | 90 | 12.8 | −3.7 |
| 225 | — | −6.04 | −3.62 | 4.63 | 4.63 | 108 | 19.4 | 4.4 |
| 226 | — | NC | NC | 5.50 | 5.50 | 96 | 19.4 | −6.8 |
| 227 | — | −6.14 | −1.63 | 4.79 | 4.85 | 99 | 19.4 | 6.6 |
| 228 | — | −5.52 | −1.41 | 3.45 | 3.47 | 140 | 15.3 | 6.1 |
| 229 | — | −6.06 | −2.96 | 4.35 | 4.35 | 152 | 19.4 | 5.1 |
| 230 | — | −5.89 | −2.20 | 3.90 | 3.91 | 146 | 15.6 | 5.6 |
| 231 | — | −5.56 | −1.00 | 3.52 | 3.59 | 140 | 15.6 | 6.6 |
| 232 | — | −6.01 | −1.42 | 4.77 | 4.84 | 82 | 19.4 | 6.7 |
| 233 | — | −5.84 | −1.54 | 4.12 | 4.16 | 119 | 15.6 | 6.3 |
| 234 | — | −6.58 | −6.58 | 4.85 | 4.96 | 125 | 19.4 | −6.8 |
| 235 | — | −5.64 | −1.58 | 4.09 | 4.11 | 129 | 15.6 | 6.1 |
| 236 | — | −5.92 | −2.36 | 4.55 | 4.55 | 135 | 19.4 | 5.6 |
| 237 | — | −6.01 | −1.07 | 4.25 | 4.47 | 152 | 19.4 | 7.2 |
| 238 | — | NC | NC | 5.83 | 5.84 | 163.84 | 13.2 | 5.8 |
| 239 | — | NC | NC | 5.45 | 5.46 | 172.63 | 13.2 | 5.5 |
| 240 | — | −6.39 | −6.39 | 3.83 | 3.83 | 142.58 | 19.4 | −2.0 |
| 241 | — | −5.82 | 0.00 | 3.49 | 3.68 | 152 | 19.4 | 7.1 |
| 242 | — | −6.16 | −2.36 | 4.59 | 4.60 | 126 | 19.4 | 5.8 |
| 243 | — | −6.94 | −3.38 | 5.79 | 5.79 | 173 | 13.1 | 5.6 |
| 244 | — | NC | NC | 5.57 | 5.57 | 108 | 13.2 | −6.8 |
| 245 | — | −5.85 | −1.21 | 4.14 | 4.22 | 119 | 15.9 | 6.7 |
| 246 | — | −6.20 | −1.44 | 4.55 | 4.66 | 126 | 19.4 | 6.9 |
| 247 | — | −5.78 | −5.78 | 3.52 | 3.52 | 90 | 13.1 | −3.6 |
| 248 | — | −6.20 | −1.44 | 4.55 | 4.66 | 126 | 19.4 | 6.9 |
| 300 | — | NC | NC | 5.76 | 5.76 | 82 | 15.1 | −7.0 |
| 301 | — | NC | NC | 4.37 | 4.37 | 64 | 15.5 | −2.6 |
| 302 | HCl | NC | NC | 2.04 | 4.27 | 69 | 19.4 | 9.7 |
| 303 | TFA | NC | NC | 4.49 | 4.49 | 82 | 19.4 | 4.3 |
| 304 | HCl | −3.81 | NC | 2.18 | 4.51 | 93 | 19.4 | 10.5 |
| 305 | — | NC | NC | 2.37 | 5.49 | 81 | 4.1 | −7.0 |
| 306 | — | NC | NC | 1.34 | 4.49 | 81 | 4.0 | −7.0 |
| 307 | HCl | −6.26 | −0.89 | 3.79 | 4.93 | 76 | 15.5 | 8.5 |
| 308 | — | NC | NC | 5.64 | 5.64 | 70 | 19.4 | −6.8 |
| 309 | — | NC | NC | 5.02 | 5.02 | 64 | 14.8 | −2.9 |
| 310 | — | NC | NC | 5.13 | 5.13 | 82 | 19.4 | 4.2 |
| 400 | — | NC | NC | 4.71 | 4.71 | 43 | 19.4 | −6.8 |
| 401 | — | NC | NC | 5.41 | 5.41 | 43 | 19.4 | −6.9 |
| 402 | — | NC | NC | 5.85 | 5.85 | 43 | 19.4 | −6.9 |
| 403 | — | NC | NC | 6.76 | 6.76 | 43 | 19.4 | −6.8 |
| 404 | — | NC | NC | 5.25 | 5.25 | 43 | 19.4 | −7.0 |
| 405 | — | NC | NC | 6.91 | 6.91 | 43 | 19.4 | −6.8 |
| 406 | — | NC | NC | 5.65 | 5.65 | 43 | 19.4 | −7.0 |
| 407 | — | −2.26 | NC | 1.49 | 4.53 | 81 | 4.2 | −6.9 |
| 408 | — | −2.60 | NC | 1.61 | 4.71 | 81 | 4.1 | −6.9 |
| 409 | HCl | −4.26 | NC | 3.64 | 4.70 | 73 | 19.4 | 8.4 |
| 410 | TFA | NC | NC | 2.83 | 3.97 | 76 | 15.7 | 8.5 |
| 411 | TFA | NC | NC | 3.01 | 4.15 | 76 | 15.6 | 8.5 |
| 412 | TFA | NC | NC | 3.93 | 5.07 | 76 | 15.7 | 8.5 |
| 413 | TFA | NC | NC | 4.74 | 5.80 | 73 | 19.4 | 8.4 |
| 414 | TFA | NC | NC | 1.43 | 1.45 | 107 | 2.0 | 8.6 |
| 415 | — | NC | NC | 3.88 | 3.88 | 81 | 19.4 | −6.5 |
| 416 | TFA | −5.67 | −5.33 | 1.73 | 1.74 | 107 | 2.1 | 9.5 |
| 417 | HCl | −2.74 | NC | 1.72 | 4.31 | 69 | 19.4 | 10.2 |
| 418 | HCl | −2.97 | NC | 2.15 | 4.75 | 69 | 19.4 | 10.2 |
| 419 | HCl | −4.63 | NC | 4.29 | 5.35 | 56 | 19.4 | 8.4 |
| 420 | — | NC | NC | 2.62 | 5.63 | 81 | 4.2 | −6.9 |
| 500 | — | NC | NC | 5.61 | 5.61 | 26 | 19.4 | −4.2 |
| 501 | — | NC | NC | 5.04 | 5.15 | 56 | 19.2 | −4.3 |
| 502 | — | NC | NC | 6.01 | 6.01 | 91 | 13.3 | −4.3 |
| 503 | — | −5.41 | −5.41 | 4.50 | 4.50 | 80 | 19.4 | −3.6 |
| 504 | — | −5.40 | NC | 4.44 | 4.44 | 65 | 19.4 | 4.6 |

TPSA = Topological Polar Surface Area; HCl = hydrochloride; TFA = trifluoroacetate; NC = not calculated.

The solubility of exemplary compounds of the present disclosure, and ganaxolone, were measured by the following protocol and provided in Table 2.2. Each test compound was weighed and placed in 1.5 mL glass vial to which an appropriate amount of 50 mM phosphate buffer at pH 7.4 was added to yield a mixture of more than 10 mg/mL. The mixture was vortexed and a magnetic stirring bar was subsequently added to the vial. The vial was placed on a magnetic stirrer with constant stirring for 16-24 hours at room temperature. The pH of the top layer solution was measured. Subsequently, 200 μL of the top layer solution was placed in a 1.5 mL microcentrifuge tube and centrifuged at 18,000 g for 10 min at room temperature. A 10 μL aliquot of supernatant was added to 990 μL of methanol:water (1/:1, v/v) and vortexed. Then, one volume of acetonitrile containing internal standard was added to one volume of the above 100-fold diluted solution and the resulting solution was vortexed and placed in 96-well plate for LC-MS/MS analysis. Calibration standards were made in a concentration range of 10 ng/mL to g/mL in methanol:water (1/:1, v/v) to which one volume of acetonitrile containing internal standard was also added, vortexed, and placed in a 96-well plate along with the solubility samples for LC-MS/MS analysis. LC-MS-MS analysis was performed utilizing multiple reaction monitoring for detection of characteristic ions for each test compound, additional related analytes and internal standard.

TABLE 2.2
Measured solubility of exemplary compounds.

Solubility

| Compound No. | pH 2.0, mg/mL | pH 7.4, ug/mL |
|---|---|---|
| ganaxolone | 0.002 | 4 |
| 111 | 1.52 | 29.1 |
| 120 | 0.039 | 35 |
| 200 | 11.8 | 29 |
| 201 | 0.036 | 33 |
| 218 | 16.9 | 152 |
| 224 | 0.024 | 26 |
| 229 | 0.022 | 11 |

### Example 120. Stability of Compounds in Simulated Gastric Fluid and Simulated Intestinal Fluid

The stability of exemplary compounds in simulated gastric fluid (SGF) and simulated intestinal fluid (SIF) was tested according to the following protocol. For each test compound, samples at a final concentration of 2 μg/mL were prepared in respective blank simulated gastric fluid (SGF) with pepsin (pH 1.2) and simulated intestinal fluid (SIF) with pancreatin (pH 6.8). All samples are incubated at 37° C. on a 150-RPM orbital shaker, and an aliquot was removed at pre-determined time points (0 to 180 minutes). Samples were precipitated with three volumes of acetonitrile containing propranolol as an internal standard, and centrifuged for 10 min at 2000 g before LC-MS/MS analysis of (the supeatant solutions.

Percent prodrug compound remaining was determined relative to 0-minute incubation samples. If a sufficient number of time points was available, the degradation half-life was obtained, based on the natural log of (compound remaining vs. time plot. The following parameters were calculated to estimate the compound stability in in vitro SGF and SIF:
1. T90=time to reach 900 prodrug compound remaining.
2. t_{1/2}=the half-life (min), where t_{1/2} is equal to 0.693/slope.

The results of the study, and general observations, are provided in Table 3 below.

TABLE 3
Stability of exemplary compounds in SGF and SIF.

| | SGF | SGF (% | Conversion | SIF | SIF (% | Conversion |
|---|---|---|---|---|---|---|
| | t_{1/2} | remain, 60 | in SGF (60 | t_{1/2} | remain, | in SIF (60 |
| Compound No. | (min) | min) | min) | (min) | 60 min) | min) |
| ganaxolone | | 95.6 | N/A | | 94.6 | N/A |
| 100 | | 90.8 | | | 93.2 | |
| 101 | | 90.5 | | | 90.2 | |
| 102 | | 56.3 | | | 74.7 | |
| 103 | | 98.4 | | | 75.9 | |
| 106 | | 2.80 | | | | |
| 109 | | 96.2 | | | 92.9 | |
| 111 | 746 | 95 | conversion to | 256 | 79.0 | no conversion |
| | | | GNX | | | to GNX |
| 120 | 250 | 83 | no conversion | 199 | 74 | no conversion |
| | | | to GNX | | | to GNX |
| 200 | | 99.0 | no conversion | | 31 | converted to |
| | | | to GNX | | | Compound |
| | | | | | | 201 |
| 201 | | | | 1583 | 98.0 | very little |
| | | | | | | conversion to |
| | | | | | | GNX |
| 218 | 2099 | 97 | no conversion | 1363 | 95 | no conversion |
| | | | to GNX | | | to GNX |
| 224 | 365 | 88 | no conversion | 98 | 60 | no conversion |
| | | | to GNX | | | to GNX |
| 228 | 511 | 89 | no conversion | 32 | 1.7 | no conversion |
| | | | to GNX | | | to GNX |
| 242 | 4595 | 99.1 | no conversion | 146 | 29 | no conversion |
| | | | to GNX | | | to GNX |
| 503 | 118.0 | 68 | conversion to | ~30 | ~90 | conversion to |
| | | | GNX | | | GNX |
| 504 | 7.41 | 0.4 | small amount | 88.4 | 49.0 | small amount |
| | | | GNX | | | GNX |
| | | | produced | | | produced |

GNX = ganaxolone.

### Example 121. Liver Microsomal Stability of Compounds

The liver microsomal stability of exemplary compounds was tested using rat, mice, dog, and human liver microsomes according to the following protocol. For several compounds only human liver microsomal stability was tested. For each test compound, samples at a final concentration of 1 μM were prepared in 25 mM potassium phosphate buffer with liver microsomes of each species at a final concentration of 0.5 mg/mL. The microsomal assays were carried out with and without the addition of NADPH at a final concentration of 1 mM. For negative control samples, NADPH reagent was not added. All samples were incubated at 37° C. on a 100-RPM orbital shaker, and an aliquot was removed at pre-determined time points. Samples were precipitated with three volume of acetonitrile containing propranolol as internal standard, and centrifuged for 10 min at 2000 g before LC/MS/MS analysis of the supernatant solutions. Note that compounds undergo only Phase I metabolism in liver microsomes.

Percent prodrug compound remaining was determined relative to 0-minute incubation samples, from which the elimination half-life was calculated based on the natural log of % compound remaining vs. time plot. The following parameters were calculated to estimate the compounds in vitro metabolic stability:
1. Cmp=concentration of microsomal proteins (mg/mL);
2. t_{1/2}=the half-life (min), where t_{1/2} is equal to 0.693/slope.
3. CLint=the intrinsic hepatic clearance (uL/min/mg), where CLint is equal to 0.693/(t_{1/2}×Cmp)

Concentrations of each compound were 0.5 μM in all liver microsome assays. Results of the assay and general observations are provided in Table 4.

TABLE 4
Rat liver microsomal stability and human liver microsomal stability results.

| | Rat Liver Microsomal | | | Human Liver | | | |
|---|---|---|---|---|---|---|---|
| | Stability | | | Microsomal Stability | | | |
| | | CLint | no | | CLint | no | |
| | | (uL/ | NADPH, | | (uL/ | NADPH, | |
| Comp. | t_{1/2} | min/mg | % remain | t_{1/2} | min/mg | % remain | |
| No. | (min) | liver) | 1 h | (min) | liver) | 1 h | Comments |
| GNX | 12.0 | 116.0 | 98.0 | 12.6 | 110.0 | 99.0 | |
| 100 | 12.7 | 109.0 | 98.0 | 11.9 | 116.0 | 100.0 | GNX not produced in human or rat |
| 101 | 6.8 | 204.0 | 100.0 | 10.2 | 137.0 | 99.0 | GNX not produced in human or rat |
| 104 | 7.41 | 187 | 99 | 12 | 116 | 98 | GNX not produced in human or rat |
| 106 | | | | 4.57 | 303 | 3.05 | GNX not produced in human |
| 107 | 21.9 | 63 | 98 | 64.7 | 21.4 | 99 | GNX not produced in human or rat |
| 108 | | | | 12.2 | 114 | 98 | GNX not produced in human |
| 110 | | | | 4.15 | 344 | 1.5 | In human GNX not produced |
| 111 | 335 | 4.13 | 99.7 | 190 | 7.28 | 82.6 | In rat no GNX produced; |
| | | | | | | | In human GNX produced |
| 112 | | | | 46.7 | 29.6 | 98.6 | In human no GNX produced |
| 114 | | | | 277 | 5.00 | 99.5 | In human GNX produced |
| 115 | | | | 13.8 | 100 | 99 | In human no GNX produced |
| 120 | 36 | 38 | 72 | 16 | 86 | 8 | In rat no GNX produced; |
| | | | | | | | In human GNX produced |
| 122 | | | | 345 | 4.02 | 99 | In human no GNX produced |
| 124 | | | | 532 | 2.61 | 99 | In human small amount of GNX produced |
| 125 | | | | 3.9 | 354 | 1 | In human GNX produced |
| 128 | | | | 9 | 153 | 20 | In human no GNX produced |
| 131 | | | | 207 | 6.7 | 82 | In human no GNX produced |
| 132 | | | | 11.7 | 118 | 1 | In human GNX produced |
| 135 | | | | 3.6 | 385 | 1.3 | In human GNX produced |
| 136 | | | | 3 | 457 | 0.5 | In human GNX produced, Compounds 132 |
| | | | | | | | and 120 also produced |
| 137 | | | | 908 | 0.5 | 95 | In human no GNX produced |
| 138 | | | | 33.2 | 41.8 | 95.3 | In human small amount of GNX produced |
| 140 | | | | 39.1 | 35.5 | 99 | In human GNX produced |
| 141 | | | | 38.3 | 36.2 | 99.7 | In human no GNX produced |
| 142 | | | | 32.3 | 42.9 | 99 | In human GNX produced |
| 200 | 12.1 | 115 | 7.5 | 6 | 229 | 1 | In rat and human, both GNX and |
| | | | | | | | Compound 201 produced |
| 201 | 32.4 | 42.7 | 99.5 | 17.4 | 79.5 | 70 | In rat, may be converted to GNX very |
| | | | | | | | slowly |
| | | | | | | | In human, GNX produced |
| 202 | | | | 2.62 | 529 | 1.85 | In human GNX produced |
| 203 | | | | 2.24 | 618 | 0.79 | In human GNX produced |
| 204 | | | | 4.12 | 337 | 10.5 | In human GNX produced |
| 205 | | | | 3.58 | 387 | 5.7 | In human GNX produced |
| 208 | | | | 37 | 37.4 | 72 | In human GNX not produced |
| 211 | | | | 10.5 | 132 | 27 | In human GNX produced, more Compound |
| | | | | | | | 201 produced than GNX |
| 212 | | | | 14.3 | 96.9 | 99 | In human small amount of GNX produced |
| 213 | | | | 27.3 | 50.7 | 98 | In human small amount of GNX produced |
| 214 | | | | 161 | 8.6 | 79.5 | In human both GNX and Compound 201 |
| | | | | | | | produced |
| 215 | | | | 257 | 5.39 | 88 | In human small amounts of Compound 201 |
| | | | | | | | and GNX produced |
| 216 | | | | 192 | 7.22 | 80 | In human both GNX and Compound 201 |
| | | | | | | | produced |
| 217 | | | | 316 | 4.39 | 99.7 | In human small amount of GNX produced |
| 218 | 56.4 | 26 | 83 | 14.4 | 95.3 | 10.0 | In rat no GNX produced; |
| | | | | | | | In human GNX produced |
| 220 | | | | 11.8 | 117 | 4 | In human small amount of GNX produced, |
| | | | | | | | small amount of Compound 201 produced |
| 222 | | | | 63.7 | 21.8 | 61 | In human no GNX produced |
| 224 | 9.8 | 142 | 1.4 | 1.67 | 828 | 0.4 | In rat no GNX produced; |
| | | | | | | | In human GNX produced |
| 227 | | | | 23.9 | 58.1 | 71 | In human no GNX produced |
| 228 | 16.4 | 84.4 | 15 | 6.2 | 225 | 6 | In rat no GNX produced; |
| | | | | | | | In human GNX produced. |
| 229 | | | | 6.1 | 227 | 1 | In human GNX produced |
| 230 | | | | 2.94 | 771 | 1 | In human GNX produced |
| 231 | | | | 5.30 | 261 | 98 | In human GNX produced |
| 232 | | | | 13.5 | 103 | 27 | In human GNX produced |
| 233 | | | | 5.3 | 261 | 5 | In human GNX produced |
| 235 | | | | 5.2 | 269 | 36 | In human GNX produced |
| 236 | | | | 3.72 | 372 | <1 | In human GNX produced |
| 237 | | | | 5.18 | 267 | <1 | In human GNX produced |
| 241 | | | | 3.9 | 356 | 3.68 | In human GNX produced |
| 242 | | | | 1.41 | 982 | 0.26 | In human GNX produced |
| 245 | | | | 11.4 | 122 | 69.7 | In human GNX produced |
| 247 | | | | 2.01 | 689 | 1.1 | In human GNX produced |
| 302 | | | | 248 | 5.59 | 99 | In human GNX not produced |
| 303 | | | | 2.48 | 599 | 90 | In human GNX not produced |
| 304 | | | | 173 | 8.03 | 83.8 | In human small amount of GNX produced |
| 305 | | | | 171 | 812 | 98.8 | In human small amount of GNX produced |
| 307 | | | | 391 | 3.55 | 99 | In human no GNX produced |
| 310 | | | | 2.9 | 474 | 1 | In human no GNX produced |
| 407 | | | | 79.8 | 17.4 | 99 | In human no GNX produced |
| 408 | | | | 143 | 9.72 | 76 | In human no GNX produced |
| 409 | | | | 12.8 | 108 | 7 | In human small amount of GNX produced |
| 410 | | | | 439 | 3.16 | 99 | In human no GNX produced |
| 411 | | | | 444 | 3.12 | 99 | In human small amount of GNX produced |
| 412 | | | | 273 | 5.07 | 94 | In human small amount of GNX produced |
| 413 | | | | 126 | 11 | 89 | In human no GNX produced |
| 414 | | | | 178 | 7.81 | 82 | In human no GNX produced |
| 415 | | | | 8.2 | 969 | 99 | In human small amount of GNX produced |
| 416 | | | | 273 | 5.08 | 99 | In human no GNX produced |
| 417 | | | | 494 | 2.08 | 99 | In human no GNX produced |
| 418 | | | | 451 | 3.08 | 99 | In human no GNX produced |
| 419 | | | | 39.5 | 35.1 | 88 | In human small amount of GNX produced |
| 420 | | | | 445 | 3.11 | 97.4 | In human no GNX produced |
| 503 | | | | 5.65 | 245 | 11.6 | In human GNX produced |
| 504 | | | | 2.5 | 554 | 0.8 | In human GNX produced |

GNX = ganaxolone.

Certain compounds were also tested in mice and dog liver microsome assays according to the protocol outlined above. The concentration of the compound was 0.5 μM in the assay. Results and general observations are provided in Table 5.

TABLE 5
Mouse liver microsomal stability and dog liver microsomal stability results.

| | Mouse Liver Microsomal Stability | | | Dog Liver Microsomal Stability | | | |
|---|---|---|---|---|---|---|---|
| | | CLint | no NADPH, | | CLint | no NADPH, | |
| Comp. | t1/2 | (uL/min/mg | % remain 1 | t1/2 | (uL/min/mg | % remain 1 | |
| No. | (min) | liver) | h | (min) | liver) | h | Comments |
| GNX | | | | 49.8 | 27.8 | 43.6 | |
| 111 | 118 | 11.8 | 92.7 | 280 | 4.95 | 92.3 | In mouse small |
| | | | | | | | amount of GNX |
| | | | | | | | produced |
| | | | | | | | In dog GNX |
| | | | | | | | produced. |
| 120 | | | | 81 | 17 | 83 | In dog GNX |
| | | | | | | | produced |
| 218 | | | | 20.1 | 69.0 | 44.4 | In dog GNX |
| | | | | | | | produced |
| 224 | | | | 7.1 | 196 | 0.6 | In dog GNX |
| | | | | | | | produced |
| 228 | | | | 10.7 | 129 | 12.9 | In dog GNX |
| | | | | | | | produced |
| 229 | | | | 6.2 | 224 | 1 | In dog GNX |
| | | | | | | | produced |
| 242 | | | | 2.07 | 668 | 0.89 | In dog GNX |
| | | | | | | | produced |

GNX = ganaxolone.

### Example 122. Plasma Stability of Compounds

The plasma stability of exemplary compounds was tested using rat, mice, dog, and human plasma, according to the following protocol. For each test compound, samples at a final concentration of 2 μg/mL were prepared in blank plasma of selected species. All samples were incubated at 37° C. on a 150-RPM orbital shaker, and an aliquot was removed at pre-determined time points (0, 15, 30, 60, and 120 minutes). Samples were precipitated with three volumes of acetonitrile containing propranolol as internal standard, and centrifuged for 10 min at 2000 g before LC/MS/MS analysis of the supernatant solutions. Percent prodrug compound remaining was determined relative to 0-minute incubation samples for each replicate, from which the degradation half-life was calculated based on the natural log of % compound remaining vs. time plot. The following parameters were calculated to estimate the compounds in vitro plasma stability:
1. t90=time to reach 9000 prodrug compound remaining.
2. t_{1/2}=the half-life (min), where t_{1/2} is equal to 0.693/slope.

Results and general observations of the assay are provided in Table 6.

TABLE 6
Rat, human, mouse, and dog plasma stability assay.

| | Rat | | Human | | Mouse | | Dog | | |
|---|---|---|---|---|---|---|---|---|---|
| | Half | | Half | | Half | | Half | | |
| | life | | life | | life | | life | | |
| Compound | (t_{1/2}) | t90 | (t1/2) | t90 | (t_{1/2}) | t90 | (t_{1/2}) | t90 | |
| No. | (min) | (min) | (min) | (min) | (min) | (min) | (min) | (min) | Observations |
| ganaxolone | 625.0 | 94.2 | 1401.0 | 211.0 | | | 3745 | 564 | |
| 100 | 185.0 | 27.9 | 184.0 | 27.8 | | | | | Stable in human, GNX not |
| | | | | | | | | | apparent in metabolites |
| 101 | 2269.0 | 342.0 | 1616.0 | 243.0 | | | | | Stable in human |
| 111 | 876 | 132 | 3602 | 542 | 446 | 67.2 | 1611 | 243 | GNX is produced in rat and |
| | | | | | | | | | mouse |
| | | | | | | | | | Very stable in human and |
| | | | | | | | | | dog (no GNX) |
| 120 | 520 | 78 | 1488 | 218 | | | 583 | 88 | No GNX produced in rat, |
| | | | | | | | | | human, or dog; |
| 200 | 9.5 | 1.4 | 14.6 | 2.19 | | | | | In rat, compound converted |
| | | | | | | | | | to GNX, not Compound |
| | | | | | | | | | 201. |
| | | | | | | | | | In human, compound not |
| | | | | | | | | | converted to GNX, but is |
| | | | | | | | | | converted to Compound |
| | | | | | | | | | 201 |
| 201 | 543 | 82 | 2076 | 313 | | | | | In rat and human, |
| | | | | | | | | | compound very slowly |
| | | | | | | | | | converted to GNX |
| 208 | | | 177 | 26.6 | | | | | No GNX produced in |
| | | | | | | | | | human |
| 218 | 56.3 | 8.5 | 56.9 | 8.6 | | | 5198 | 783 | No GNX produced in |
| | | | | | | | | | human or dog; |
| | | | | | | | | | GNX produced in rat |
| 220 | | | 21.1 | 3.18 | | | | | In human no GNX |
| | | | | | | | | | produced |
| 224 | 14.4 | 2.16 | 43 | 6.5 | | | 838 | 126 | No GNX produced in |
| | | | | | | | | | human or dog; |
| | | | | | | | | | GNX produced in rat |
| 228 | 12 | 1.8 | 9 | 1.4 | | | 20 | 3 | No GNX produced in |
| | | | | | | | | | human or dog; Compound |
| | | | | | | | | | 224 produced in human |
| | | | | | | | | | GNX produced in rat |
| 242 | | | 33 | 4.99 | | | 3842 | 578 | No GNX produced in |
| | | | | | | | | | human or dog |
| 503 | | | <<30 | | | | | | In human amount of |
| | | | | | | | | | Compound 503 at |
| | | | | | | | | | start: 3883 nM. Amount at |
| | | | | | | | | | end: 0.2 nM. Amount of |
| | | | | | | | | | GNX at start 349 nM. |
| | | | | | | | | | Amount at end 2469 nM |
| 504 | | | 17.1 | 2.54 | | | | | In human very small |
| | | | | | | | | | amount of GNX produced |

GNX = ganaxolone

### Example 123. Pharmacokinetic Study with Compound 111

A pharmacokinetic study was carried out using Compound 111, comparing intravenous administration (5 mg/kg; n=3) and oral (PO) administration (25 mg/kg; n=3) in fasted male beagle dogs. A single dose of Compound 111 was administered either orally or intravenously, with the compound formulated in the following vehicle: 1% DMA, 48% CAPTISOL® (30% concentration), 1% TWEEN-20® (10% concentration), 25% CAPTISOL® (30% concentration), and 25% PEG-300. Analysis time points for the IV arm were 0.083, 0.25, 0.5, 1, 2, 4, 8, 12, 24, 36 and 48 hours post dose and analysis time points for the PO arm were 0.25, 0.5, 1, 2, 4, 8, 12, 24, 36 and 48 hours post dose. Pharmacokinetic parameters were obtained for both Compound 111 (prodrug) and ganaxolone (metabolite), with the expectation of observing the appearance of ganaxolone in the dog plasma following conversion of Compound 111 by metabolic processes.

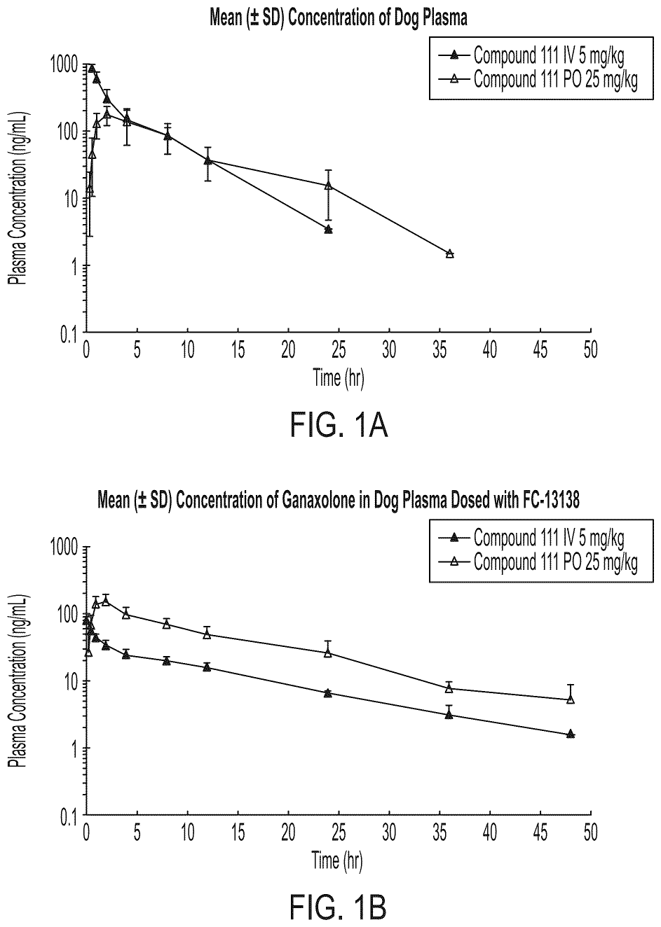

The results are provided below in Tables 7 and 8, and in the plasma concentration-over-time curves provided as FIGS. 1A and 1B.

TABLE 7
Pharmacokinetic parameters for Compound 111 after IV (5
mg/kg) or oral (25 mg/kg) administration of Compound 111.
Compound 111 PK (after IV admin. of Compound 111)

| | t_{1/2} | C₀ | AUCₗₐₛₜ | AUC_{inf} | V_{z} | Vₛₛ | CL | MRT_{Inf} |
|---|---|---|---|---|---|---|---|---|
| | (hr) | (ng/mL) | (hr*ng/mL) | (hr*ng/mL) | (L/kg) | (L/kg) | (mL/min/kg) | (hr) |
| Mean | 3.54 | 1505 | 2743 | 2786 | 9.47 | 6.71 | 31.7 | 3.68 |

Compound 111 PK (after oral admin. of Compound 111)

| | t_{1/2} | Tₘₐₓ | Cₘₐₓ | AUCₗₐₛₜ | AUC_{Inf} | MRT_{Inf} | AUC_{Inf}/D | |
|---|---|---|---|---|---|---|---|---|
| | (hr) | (hr) | (ng/mL) | (hr*ng/mL) | (hr*ng/mL) | (hr) | (hr*kg*ng/mL/mg) | F (%) |
| Mean | 7.15 | 2.67 | 177 | 1566 | 1716 | 10.6 | 68.7 | 11.4 |

TABLE 8
Pharmacokinetic parameters for ganaxolone after IV (5 mg/kg)
or oral (25 mg/kg) administration of Compound 111.
Ganaxolone PK (after IV admin. of Compound 111)

| | t_{1/2} | Tₘₐₓ | Cₘₐₓ | AUCₗₐₛₜ | AUC_{Inf} | MRT_{Inf} | Cₘₐₓ ratio | AUCₗₐₛₜ ratio |
|---|---|---|---|---|---|---|---|---|
| | (hr) | (hr) | (ng/mL) | (hr*ng/mL) | (hr*ng/mL) | (hr) | (metabolite/prodrug) | (metabolite/prodrug) |
| Mean | 13.7 | 0.0833 | 81.1 | 521 | 567 | 15.9 | 0.0599 | 0.201 |

Ganaxolone PK (after oral admin. of Compound 111)

| | t_{1/2} | Tₘₐₓ | Cₘₐₓ | AUCₗₐₛₜ | AUC_{Inf} | MRT_{Inf} | Cₘₐₓ ratio | AUCₗₐₛₜ ratio |
|---|---|---|---|---|---|---|---|---|
| | (hr) | (hr) | (ng/mL) | (hr*ng/mL) | (hr*ng/mL) | (hr) | (metabolite/prodrug) | (metabolite/prodrug) |
| Mean | 10.1 | 1.33 | 150 | 1724 | 1802 | 14.3 | 0.859 | 1.21 |

### Example 124. Pharmacokinetic Study with Compound 224

A pharmacokinetic study was carried out using Compound 224, comparing intravenous (IV) administration (2 mg/kg; n=3) and oral (PO) administration (10 mg/kg; n=3) in fasted male beagle dogs. A single dose of Compound 224 was administered either orally or intravenously, with the compound formulated in the following vehicle: 1% DMA/1% of 10% Tween-20/25% of PEG400/73% of 30% kleptose. Analysis time points for the IV arm were 0.083, 0.25, 0.5, 1, 2, 4, 8, 12, 24, 36 and 48 hours post dose and analysis time points for the PO arm were 0.25, 0.5, 1, 2, 4, 8, 12, 24, 36 and 48 hours post dose. Pharmacokinetic parameters were obtained for both Compound 224 (prodrug) and ganaxolone (metabolite), with the expectation of observing the appearance of ganaxolone in the dog plasma following conversion of Compound 224 by metabolic processes.

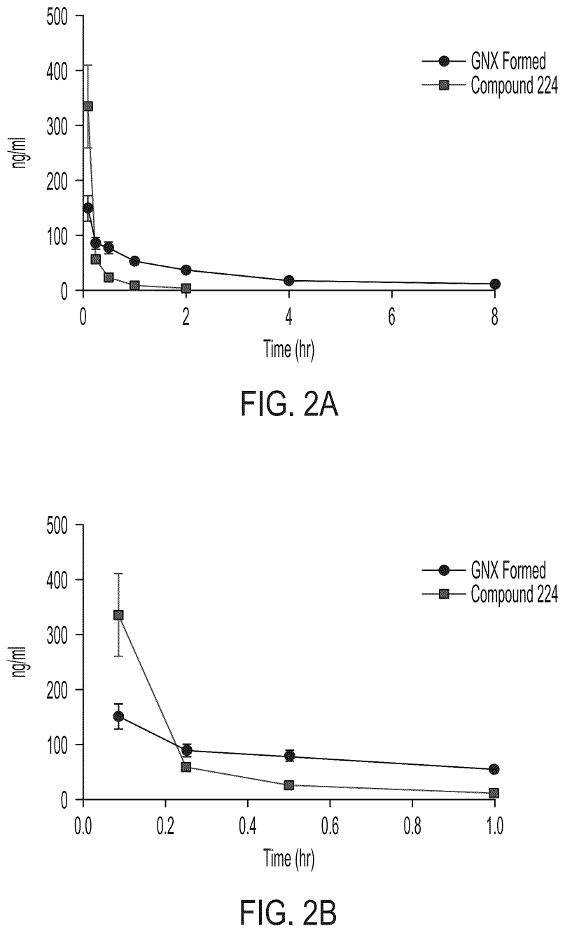

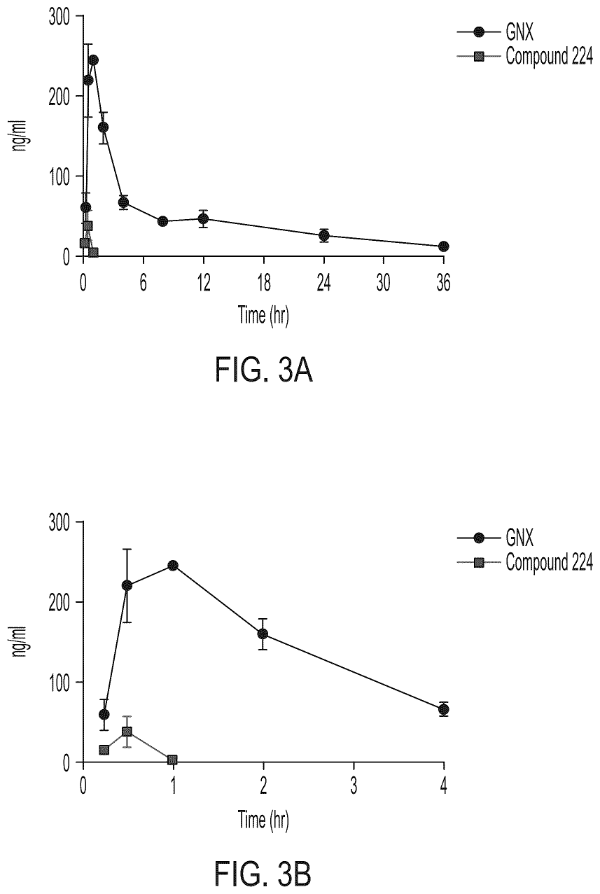

The results are provided below in Tables 9 and 10, and in the plasma concentration-over-time curves provided as FIGS. 2A, 2B, 3A, and 3B.

TABLE 9
Pharmacokinetic parameters for Compound 224 after IV (2
mg/kg) or oral (10 mg/kg) administration of Compound 224.
Compound 224 PK (after IV admin. of Compound 224)

| | t_{1/2} | C₀ | AUCₗₐₛₜ | AUC_{inf} | V_{z} | Vₛₛ | CL | MRT_{Inf} |
|---|---|---|---|---|---|---|---|---|
| | (hr) | (ng/mL) | (hr*ng/mL) | (hr*ng/mL) | (L/kg) | (L/kg) | (mL/min/kg) | (hr) |
| Mean | .0595 | 833 | 105 | 109 | 16.1 | 5.92 | 340 | 0.289 |

Compound 224 PK (after oral admin. of Compound 224)

| | t_{1/2} | Tₘₐₓ | Cₘₐₓ | AUCₗₐₛₜ | AUC_{Inf} | MRT_{Inf} | AUC_{Inf}/D | |
|---|---|---|---|---|---|---|---|---|
| | (hr) | (hr) | (ng/mL) | (hr*ng/mL) | (hr*ng/mL) | (hr) | (hr*kg*ng/mL/mg) | F (%) |
| Mean | NA | 0.5 | 38.3 | 19.3 | NA | NA | NA | 3.67 |

TABLE 10
Pharmacokinetic parameters for ganaxolone after IV (2 mg/kg)
or oral (10 mg/kg) administration of Compound 224.
Ganaxolone PK (after IV admin. of Compound 224)

| | t_{1/2} | Tₘₐₓ | Cₘₐₓ | AUCₗₐₛₜ | AUC_{Inf} | MRT_{Inf} | Cₘₐₓ ratio | AUCₗₐₛₜ ratio |
|---|---|---|---|---|---|---|---|---|
| | (hr) | (hr) | (ng/mL) | (hr*ng/mL) | (hr*ng/mL) | (hr) | (metabolite/prodrug) | (metabolite/prodrug) |
| Mean | 13.3 | .0833 | 149 | 419 | 475 | 14.6 | 0.467 | 4.36 |

Ganaxolone PK (after oral admin. of Compound 224)

| | t_{1/2} | Tₘₐₓ | Cₘₐₓ | AUCₗₐₛₜ | AUC_{Inf} | MRT_{Inf} | Cₘₐₓ ratio | AUCₗₐₛₜ ratio |
|---|---|---|---|---|---|---|---|---|
| | (hr) | (hr) | (ng/mL) | (hr*ng/mL) | (hr*ng/mL) | (hr) | (metabolite/prodrug) | (metabolite/prodrug) |
| Mean | 15.5 | .0833 | 265 | 1761 | 1960 | 18.8 | 7.61 | 100 |

### Example 125. Pharmacokinetic Study with Compound 218

A pharmacokinetic study was carried out using Compound 218, comparing intravenous (IV) administration (5 mg/kg; n=3) and oral (PO) administration (25 mg/kg; n=3) in fasted male beagle dogs. A single dose of Compound 218 was administered either orally or intravenously, with the compound formulated in the following vehicle: 1% DMA/1% of 1% Tween-20/25% of PEG400/73% of 30% Captisol. Analysis time points for the IV arm were 0.083, 0.25, 0.5, 1, 2, 4, 8, 12, 24, 36 and 48 hours post dose and analysis time points for the PO arm were 0.25, 0.5, 1, 2, 4, 8, 12, 24, 36 and 48 hours post dose. Pharmacokinetic parameters were obtained for both Compound 218 (prodrug) and ganaxolone (metabolite), with the expectation of observing the appearance of ganaxolone in the dog plasma following conversion of Compound 218 by metabolic processes.

The volume of distribution (Vd) (dose/Co) was 1.82 L/kg.

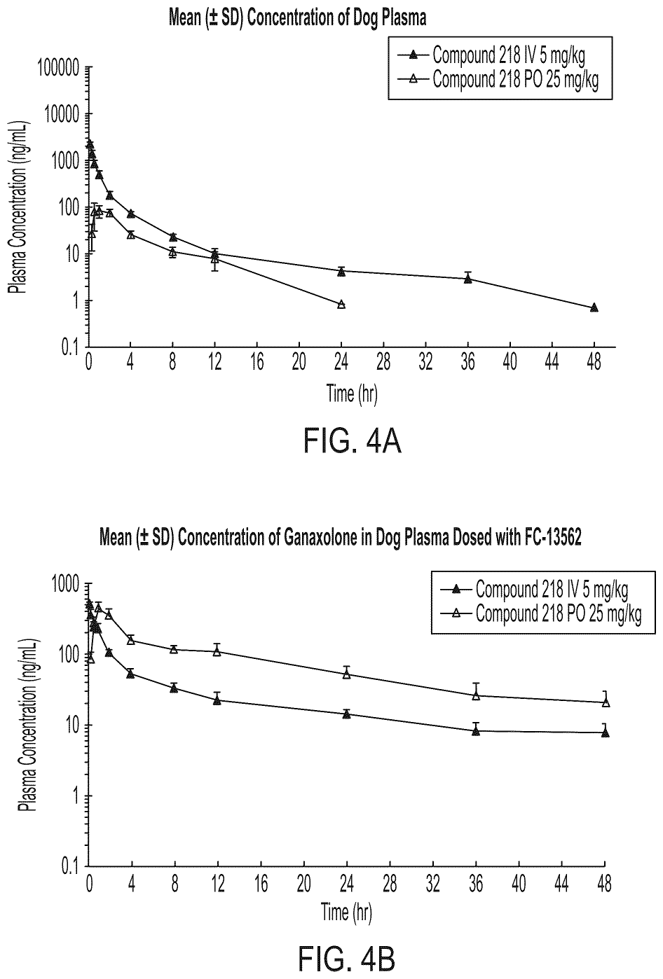

The results are provided below in Tables 11 and 12, and in the plasma concentration-over-time curves provided as FIGS. 4A and 4B.

TABLE 11
Pharmacokinetic parameters for Compound 218 after IV (5
mg/kg) or oral (25 mg/kg) administration of Compound 218.
Compound 218 PK (after IV admin. of Compound 218)

| | t_{1/2} | C₀ | AUCₗₐₛₜ | AUC_{inf} | V_{z} | Vₛₛ | CL | MRT_{Inf} |
|---|---|---|---|---|---|---|---|---|
| | (hr) | (ng/mL) | (hr*ng/mL) | (hr*ng/mL) | (L/kg) | (L/kg) | (mL/min/kg) | (hr) |
| Mean | 17.7 | 2754 | 2127 | 2204 | 57.5 | 11.4 | 37.9 | 5.03 |

Compound 218 PK (after oral admin. of Compound 218)

| | t_{1/2} | Tₘₐₓ | Cₘₐₓ | AUCₗₐₛₜ | AUC_{Inf} | MRT_{Inf} | | |
|---|---|---|---|---|---|---|---|---|
| | (hr) | (hr) | (ng/mL) | (hr*ng/mL) | (hr*ng/mL) | (hr) | F (%) | — |
| Mean | 6.13 | 1.17 | 89.8 | 359 | 415 | 6.38 | 3.76 | — |

TABLE 12
Pharmacokinetic parameters for ganaxolone after IV (5 mg/kg)
or oral (25 mg/kg) administration of Compound 218.
Ganaxolone PK (after IV admin. of Compound 218)

| | t_{1/2} | Tₘₐₓ | Cₘₐₓ | AUCₗₐₛₜ | AUC_{Inf} | MRT_{Inf} | Cₘₐₓ ratio | AUCₗₐₛₜ ratio |
|---|---|---|---|---|---|---|---|---|
| | (hr) | (hr) | (ng/mL) | (hr*ng/mL) | (hr*ng/mL) | (hr) | (metabolite/prodrug) | (metabolite/prodrug) |
| Mean | 29.4 | 0.0833 | 507 | 1348 | 1715 | 27.8 | 0.23 | 0.633 |

Ganaxolone PK (after oral admin. of Compound 218)

| | t_{1/2} | Tₘₐₓ | Cₘₐₓ | AUCₗₐₛₜ | AUC_{Inf} | MRT_{Inf} | Cₘₐₓ ratio | AUCₗₐₛₜ ratio |
|---|---|---|---|---|---|---|---|---|
| | (hr) | (hr) | (ng/mL) | (hr*ng/mL) | (hr*ng/mL) | (hr) | (metabolite/prodrug) | (metabolite/prodrug) |
| Mean | 17.6 | 1 | 441 | 3839 | 4397 | 20.1 | 5.58 | 10.8 |

### Example 126. Pharmacokinetic Study with Compound 242

A pharmacokinetic study will be carried out using Compound 242, comparing intravenous (IV) administration (3 mg/kg; n=3) and intramuscular (IM) administration (2.5 mg/kg; n=3) in fasted male beagle dogs. A single dose of Compound 242 will be administered either intramuscular or intravenously, with the compound formulated in the following vehicle: 2% DMA, 1% of 1% tween, 32% of PEG400, 65% of 8% captisol. Analysis time points for the IV and IM arms will be 0.083, 0.25, 0.5, 1, 2, 4, 8, 12, 24, 36 and 48 hours post dose. Pharmacokinetic parameters will be obtained for both Compound 242 (prodrug) and ganaxolone (metabolite), with the expectation of observing the appearance of ganaxolone in the dog plasma following conversion of Compound 242 by metabolic processes.

What is claimed is:

1. A compound of Formula (I): 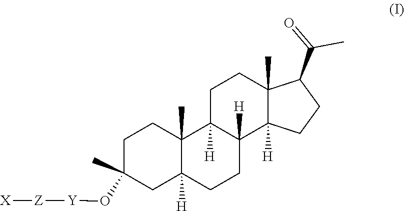
or a pharmaceutically acceptable salt, solvate, hydrate, tautomer, or stereoisomer thereof, wherein
Y is —C(O)— or —C(R^{A})(R^{B})— (e.g., —CH₂—);
Z is absent, alkylene, alkenylene, alkynylene, heteroalkylene, cycloalkylene, heterocyclylene, arylene, heteroarylene, —NR¹—, —O—, or —S—, wherein the alkylene, alkenylene, alkynylene, or heteroalkylene is optionally substituted;
X is alkyl, alkenyl, alkynyl, heteroalkyl, cycloalkyl, heterocyclyl, aryl, heteroaryl, haloalkyl, alkylene-cycloalkyl, alkylene-heterocyclyl, alkylene-aryl, alkylene-heteroaryl, heteroalkylene-cycloalkyl, heteroalkylene-heterocyclyl, heteroalkylene-aryl, heteroalkylene-heteroaryl, cycloalkylene-alkyl, cycloalkylene-heteroalkyl, heterocyclylene-alkyl, heterocyclylene-heteroalkyl, —OC(O)-alkyl, —C(O)O-alkyl, —OC(O)-heteroalkyl, —C(O)O— heteroalkyl, —N₃, —NR¹R², —OR³, or —SR⁴, wherein the alkyl, alkylene, alkenyl, alkynyl, heteroalkyl, heteroalkylene, cycloalkyl, cycloalkylene, heterocyclyl, heterocyclylene, aryl, or heteroaryl is optionally substituted;
R¹ and R² are each independently hydrogen, alkyl, alkenyl, alkynyl, heteroalkyl, cycloalkyl, heterocyclyl, aryl, heteroaryl, alkylene-cycloalkyl, alkylene-heterocyclyl, alkylene-aryl, alkylene-heteroaryl, heteroalkylene-cycloalkyl, heteroalkylene-heterocyclyl, heteroalkylene-aryl, heteroalkylene-heteroaryl, cycloalkylene-alkyl, cycloalkylene-heteroalkyl, heterocyclylene-alkyl, or heterocyclylene-heteroalkyl, wherein the alkyl, alkylene, alkenyl, alkynyl, heteroalkyl, heteroalkylene, cycloalkyl, cycloalkylene, heterocyclyl, heterocyclylene, aryl, or heteroaryl is optionally substituted;
or R¹ and R², together with the atoms to which they are attached, form a 3-7 membered cycloalkyl, heterocyclyl, aryl, or heteroaryl, wherein the cycloalkyl, heterocyclyl, aryl, or heteroaryl is optionally substituted;
R³ is hydrogen, alkyl, alkenyl, alkynyl, heteroalkyl, cycloalkyl, heterocyclyl, aryl, heteroaryl, alkylene-cycloalkyl, alkylene-heterocyclyl, alkylene-aryl, alkylene-heteroaryl, heteroalkylene-cycloalkyl, heteroalkylene-heterocyclyl, heteroalkylene-aryl, heteroalkylene-heteroaryl, cycloalkylene-alkyl, cycloalkylene-heteroalkyl, heterocyclylene-alkyl, or heterocyclylene-heteroalkyl, wherein the alkyl, alkylene, alkenyl, alkynyl, heteroalkyl, heteroalkylene, cycloalkyl, cycloalkylene, heterocyclyl, heterocyclylene, aryl, or heteroaryl is optionally substituted;
R⁴ is hydrogen, alkyl, alkenyl, alkynyl, heteroalkyl, cycloalkyl, heterocyclyl, aryl, heteroaryl, alkylene-cycloalkyl, alkylene-heterocyclyl, alkylene-aryl, alkylene-heteroaryl, heteroalkylene-cycloalkyl, heteroalkylene-heterocyclyl, heteroalkylene-aryl, heteroalkylene-heteroaryl, wherein the alkyl, alkylene, alkenyl, alkynyl, heteroalkyl, heteroalkylene, cycloalkyl, cycloalkylene, heterocyclyl, heterocyclylene, aryl, or heteroaryl is optionally substituted; and
R^{A} and R^{B} are each independently hydrogen, alkyl, heteroalkyl, cycloalkyl, heterocyclyl, aryl, or heteroaryl, wherein the alkyl, heteroalkyl, cycloalkyl, heterocyclyl, aryl, or heteroaryl is optionally substituted.

2. The compound of claim 1, wherein Y is —C(O)—.

3. The compound of claim 1 or 2, wherein Z is methylene (e.g., unsubstituted methylene), 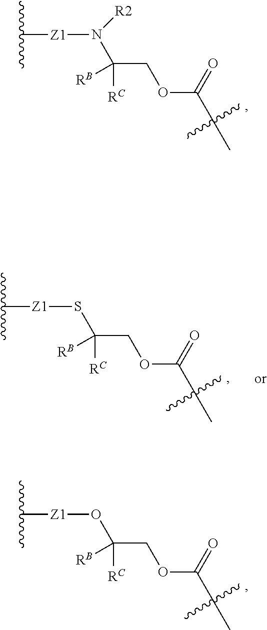
wherein
Z1 is absent, alkylene (e.g., methylene, e.g., unsubstituted methylene), alkenylene, alkynylene, heteroalkylene, cycloalkylene, heterocyclylene, arylene, or heteroarylene, wherein the alkylene, alkenylene, alkynylene, or heteroalkylene is optionally substituted;
R² is hydrogen or alkyl;
R^{B} and R^{C} are each independently hydrogen, alkyl, heteroalkyl, cycloalkyl, heterocyclyl, aryl, or heteroaryl, wherein the alkyl, heteroalkyl, cycloalkyl, heterocyclyl, aryl, or heteroaryl is optionally substituted; and
each [symbol] denotes the point of attachment of Z to X or Y.

4. The compound of any one of the preceding claims, wherein X is a moiety selected from the group consisting of 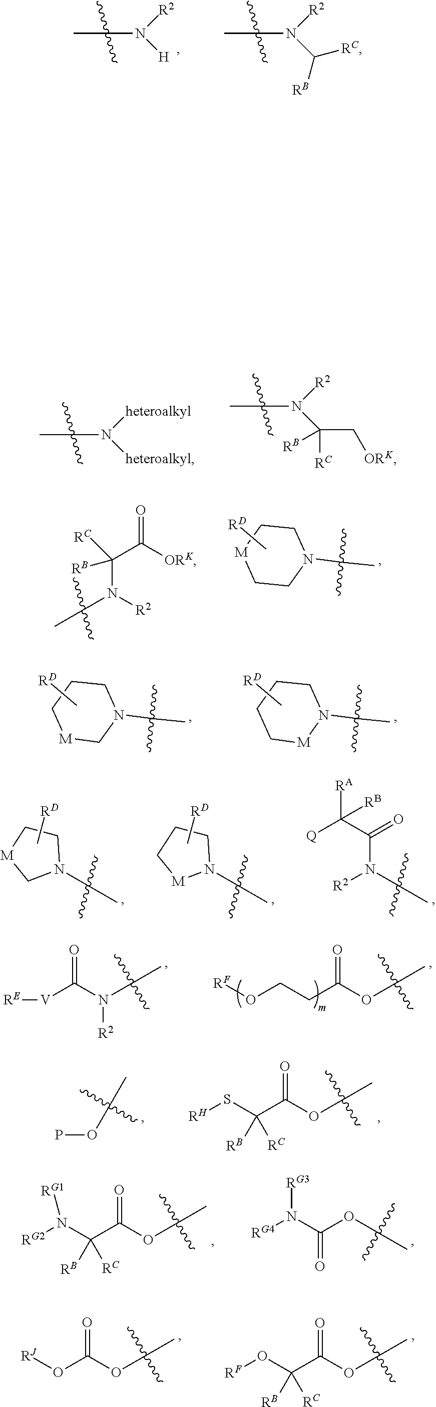 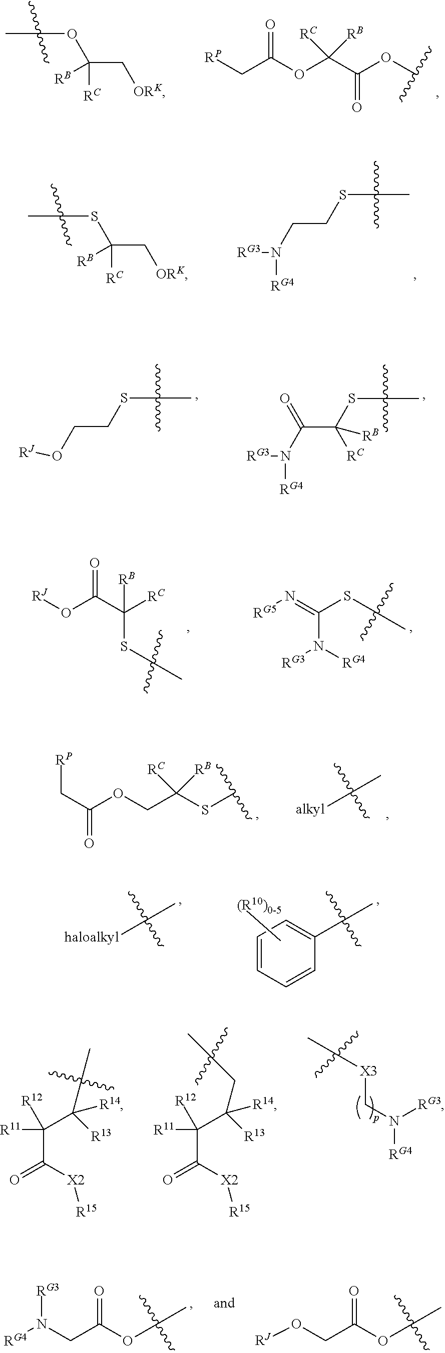
wherein
R² is hydrogen or alkyl;
M is-C(H)(R^{D})— (e.g., —CH₂—), —O—, or —N(R^{D})— (e.g., —N(Me)—);
P is an amino acid residue (e.g., glycine, alanine, valine, leucine, or proline), that may be a D-amino acid residue or L-amino acid residue, and is optionally substituted (e.g., at a terminal nitrogen atom, e.g., by a carbonate group, e.g., a carboxybenzyl (Cbz) group),
or P is a dipeptide moiety (e.g., a leucine-leucine moiety) that may comprise D-amino acids, L-amino acids, or a combination thereof, and is optionally substituted (e.g., at one or more nitrogen atoms, e.g., at a terminal nitrogen atom, e.g., by a carbonate group, e.g., a carboxybenzyl (Cbz) group);
Q is heteroalkyl or heterocyclyl, wherein the heteroalkyl or heterocyclyl is optionally substituted;
V is —O—, —N(H)—, or —N(Me)—;
X2 and X3 are each independently —CH₂—, —O—, —S—, or —N(R^{G3})— (e.g., —NH—);
R^{A}, R^{B}, R^{C}, R^{D}, R^{E}, and R^{K} are each independently hydrogen, alkyl, heteroalkyl, cycloalkyl, heterocyclyl, aryl, or heteroaryl, wherein the alkyl, heteroalkyl, cycloalkyl, heterocyclyl, aryl, or heteroaryl is optionally substituted;
R^{F} and R^{H} are each independently hydrogen or alkyl;
R^{G1} and R^{G2} are each independently hydrogen, alkyl, or an amino acid residue (e.g., glycine, alanine, valine, leucine or proline), that may be a D-amino acid residue or L-amino acid residue, and is optionally substituted (e.g., at a terminal nitrogen atom, e.g., by a carbonate group, e.g., a carboxybenzyl (Cbz) group),
or R^{G1} and R^{G2}, together with the atoms to which they are attached, form a 3-7 membered cycloalkyl, heterocyclyl, aryl, or heteroaryl, wherein the cycloalkyl, heterocyclyl, aryl, or heteroaryl is optionally substituted;
R^{G3} and R^{G4} are each independently hydrogen, alkyl, or heteroalkyl, wherein the alkyl or heteroalkyl is optionally substituted;
or R^{G3} and R^{G4}, together with the atoms to which they are attached, form a 3-7 membered cycloalkyl, heterocyclyl, aryl, or heteroaryl, wherein the cycloalkyl, heterocyclyl, aryl, or heteroaryl is optionally substituted;
R^{G5} is hydrogen, alkyl, or heteroalkyl, wherein the alkyl or heteroalkyl is optionally substituted;
R^{J} is hydrogen, alkyl, alkylene-heterocyclyl, heteroalkyl, or heterocyclyl, wherein the alkyl, alkylene, heteroalkyl, or heterocyclyl is optionally substituted;
R^{P} is halo, —NH-alkyl, —NH-heteroalkyl, —N-(alkyl)₂ (e.g., —N(Me)₂), —N-(heteroalkyl)₂ (e.g., —N(CH₂CH₂OH)₂), or heterocyclyl (e.g., morpholinyl);
R¹⁰ is alkyl, alkenyl, alkynyl, haloalkyl, halo, —OR, —N(R^{G3})(R^{G4}); —NO₂, —CN, or —NC;
R¹¹, R¹², R¹³, and R¹⁴ are each independently hydrogen, alkyl, alkenyl, alkynyl, heteroalkyl, haloalkyl, halo, or —N(R^{G3})(R^{G4}), wherein the alkyl, alkenyl, alkynyl, heteroalkyl, or haloalkyl is optionally substituted,
or one of R¹¹ and R¹², and one of R¹³ and R¹⁴, together with the atoms to which they are attached, form a 3-7 membered cycloalkyl, heterocyclyl, aryl, or heteroaryl, wherein the cycloalkyl, heterocyclyl, aryl, or heteroaryl is optionally substituted;
R¹⁵ is hydrogen, alkyl, alkenyl, alkynyl, heteroalkyl, haloalkyl, or halo; wherein the alkyl, alkenyl, alkynyl, heteroalkyl, or haloalkyl is optionally substituted; and
m is an integer of 1-5;
p is an integer between 0 and 4; and [symbol]
denotes the point of attachment of X to Z, or X to Y when Z is absent.

5. The compound of any one of the preceding claims, wherein X is a moiety selected from the group consisting of 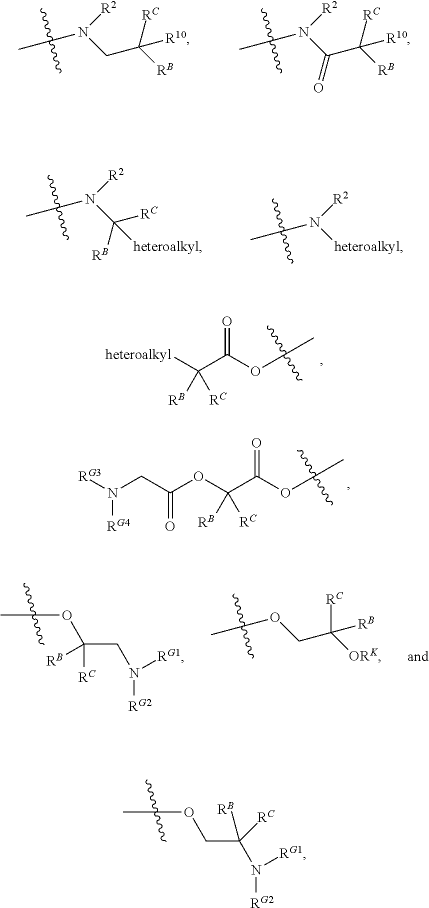
wherein
R^{B}, R^{C}, and R^{K} are each independently hydrogen, alkyl, heteroalkyl, cycloalkyl, heterocyclyl, aryl, or heteroaryl, wherein the alkyl, heteroalkyl, cycloalkyl, heterocyclyl, aryl, or heteroaryl is optionally substituted;
R^{G1} and R^{G2} are each independently hydrogen, alkyl, or an amino acid residue (e.g., glycine, alanine, valine, leucine or proline), that may be a D-amino acid residue or L-amino acid residue, and is optionally substituted (e.g., at a terminal nitrogen atom, e.g., by a carbonate group, e.g., a carboxybenzyl (Cbz) group),
or R^{G1} and R^{G2}, together with the atoms to which they are attached, form a 3-7 membered cycloalkyl, heterocyclyl, aryl, or heteroaryl, wherein the cycloalkyl, heterocyclyl, aryl, or heteroaryl is optionally substituted;
R^{G3} and R^{G4} are each independently hydrogen, alkyl, or heteroalkyl, wherein the alkyl or heteroalkyl is optionally substituted;
or R^{G3} and R^{G4}, together with the atoms to which they are attached, form a 3-7 membered cycloalkyl, heterocyclyl, aryl, or heteroaryl, wherein the cycloalkyl, heterocyclyl, aryl, or heteroaryl is optionally substituted;
R² is hydrogen or alkyl; and
R¹⁰ is alkyl, alkenyl, alkynyl, haloalkyl, halo, —OR, —N(R^{G3})(R^{G4}); —NO₂, —CN, or —NC.

6. The compound of any one of the preceding claims, wherein Y is —C(O)— and Z is alkylene (e.g., methylene).

7. The compound of any one of the preceding claims, wherein R¹ and R² are not both hydrogen.

8. A compound of Formula (I-II): 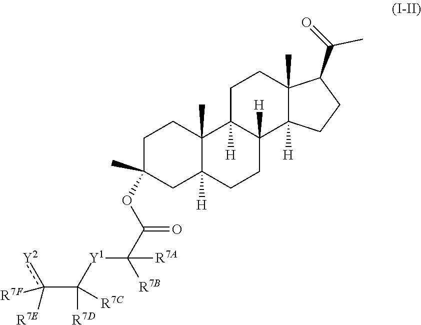
or a pharmaceutically acceptable salt, solvate, hydrate, tautomer, or stereoisomer thereof, wherein: [symbol]
denotes a single bond or a double bond;
Y¹ is alkylene (e.g., methylene), alkenylene, alkynylene, heteroalkylene, —C(O)—, —OC(O)—, —C(O)O—, —NR¹—, —O—, or —S—, wherein the alkylene, alkenylene, alkynylene, or heteroalkylene is optionally substituted;
Y² is heteroalkyl, heterocyclyl, heteroaryl, heteroalkylene-cycloalkyl, heteroalkylene-heterocyclyl, heteroalkylene-aryl, heteroalkylene-heteroaryl, —NR¹R², —OR³, —SR⁴, ═NR¹, or ═O, wherein the heteroalkyl, cycloalkyl, heterocyclyl, aryl, or heteroaryl is optionally substituted;
R¹ and R² are each independently hydrogen, alkyl, alkenyl, alkynyl, heteroalkyl, cycloalkyl, heterocyclyl, aryl, heteroaryl, alkylene-cycloalkyl, alkylene-heterocyclyl, alkylene-aryl, alkylene-heteroaryl, heteroalkylene-cycloalkyl, heteroalkylene-heterocyclyl, heteroalkylene-aryl, heteroalkylene-heteroaryl, cycloalkylene-alkyl, cycloalkylene-heteroalkyl, heterocyclylene-alkyl, or heterocyclylene-heteroalkyl, wherein the alkyl, alkylene, alkenyl, alkynyl, heteroalkyl, heteroalkylene, cycloalkyl, cycloalkylene, heterocyclyl, heterocyclylene, aryl, or heteroaryl is optionally substituted;
or R¹ and R², together with the atoms to which they are attached, form a 3-7 membered cycloalkyl, heterocyclyl, aryl, or heteroaryl, wherein the cycloalkyl, heterocyclyl, aryl, or heteroaryl is optionally substituted;
R³ is hydrogen, alkyl, alkenyl, alkynyl, heteroalkyl, cycloalkyl, heterocyclyl, aryl, heteroaryl, alkylene-cycloalkyl, alkylene-heterocyclyl, alkylene-aryl, alkylene-heteroaryl, heteroalkylene-cycloalkyl, heteroalkylene-heterocyclyl, heteroalkylene-aryl, heteroalkylene-heteroaryl, cycloalkylene-alkyl, cycloalkylene-heteroalkyl, heterocyclylene-alkyl, or heterocyclylene-heteroalkyl, wherein the alkyl, alkylene, alkenyl, alkynyl, heteroalkyl, heteroalkylene, cycloalkyl, cycloalkylene, heterocyclyl, heterocyclylene, aryl, or heteroaryl is optionally substituted;
R⁴ is hydrogen, alkyl, alkenyl, alkynyl, heteroalkyl, cycloalkyl, heterocyclyl, aryl, heteroaryl, alkylene-cycloalkyl, alkylene-heterocyclyl, alkylene-aryl, alkylene-heteroaryl, heteroalkylene-cycloalkyl, heteroalkylene-heterocyclyl, heteroalkylene-aryl, heteroalkylene-heteroaryl, wherein the alkyl, alkylene, alkenyl, alkynyl, heteroalkyl, heteroalkylene, cycloalkyl, cycloalkylene, heterocyclyl, heterocyclylene, aryl, or heteroaryl is optionally substituted;
R^{7A} and R^{7B} are each independently hydrogen or optionally substituted alkyl, or one of R^{7A} and R^{7B} is bonded to Y¹ through one or more carbon atoms or heteroatoms to form a cyclic moiety (e.g., cycloalkyl, heterocyclyl, aryl, or heteroaryl, e.g., pyrrole), and the other of the R^{7A} and R^{7B} groups is hydrogen or optionally substituted alkyl;
R^{7C} and R^{7D} are each independently hydrogen or optionally substituted alkyl, or R^{7C} and R^{7D} are each bonded to the same oxygen atom to form an oxo group; and
R^{7E} and R^{7F} are each independently absent, hydrogen, alkyl, heteroalkyl, —NR¹R², —OR³, or —SR⁴, wherein the alkyl or heteroalkyl is optionally substituted, or R^{7E} and R^{7F} are each bonded to the same oxygen to form an oxo group, with the proviso that when [symbol] is a double bond, one of R^{7E} and R^{7F} is absent.

9. The compound of claim 8, wherein Y¹ is —O— or —N(R¹)— (e.g., —N(H)—, —N(alkyl)-, or —N(heteroalkyl)-).

10. The compound of claim 8 or 9, selected from the group consisting of: 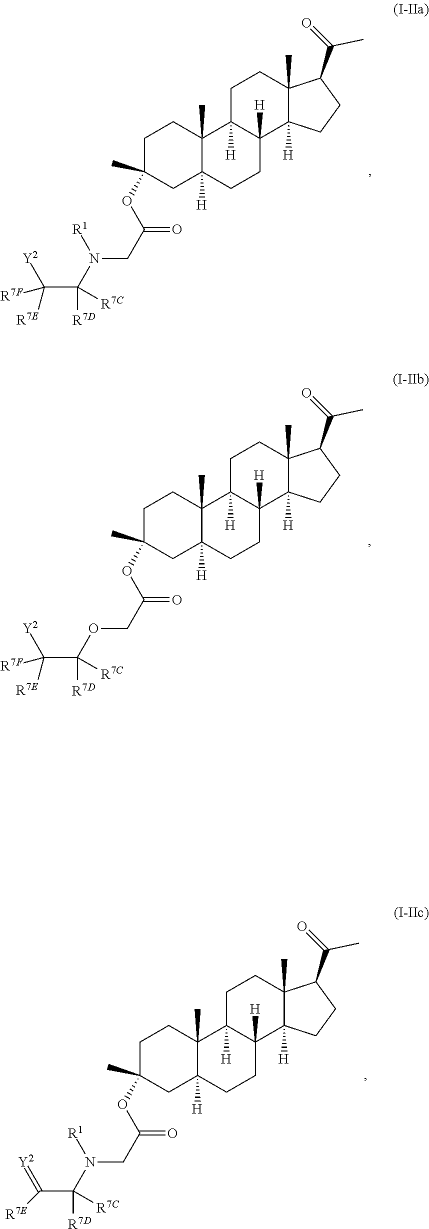 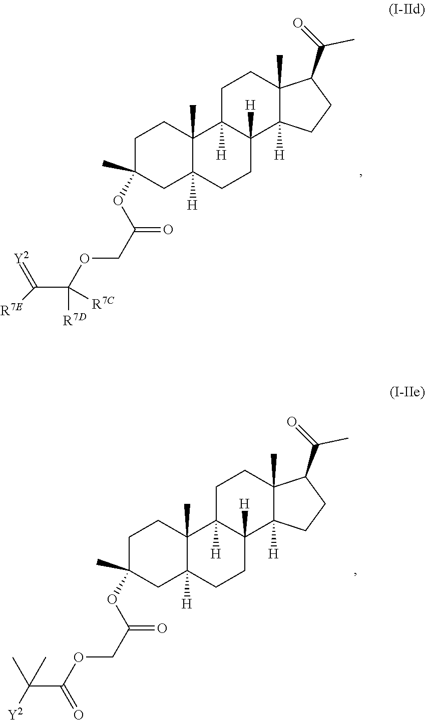
and pharmaceutically acceptable salts, solvates, hydrates, tautomers, and stereoisomer thereof, wherein Y², R¹, R^{7C}, R^{7D}, R^{7E}, and R^{7F} are as defined in claim 8.

11. The compound of any one of claims 8-10, wherein Y², or R^{7E} when [symbol] is a double bond, is optionally substituted heteroalkyl, —NR¹R² (e.g., —NH₂, —NHMe, or —NMe₂), —OR³ (e.g., —OH, —OMe, —OEt, or —OtBu), or ═O, wherein R¹, R², and R³ are as defined in claim 8.

12. The compound of any one of claims 8-11, wherein Y², or R^{7E} when [symbol] is a double bond, is selected from the group consisting of:

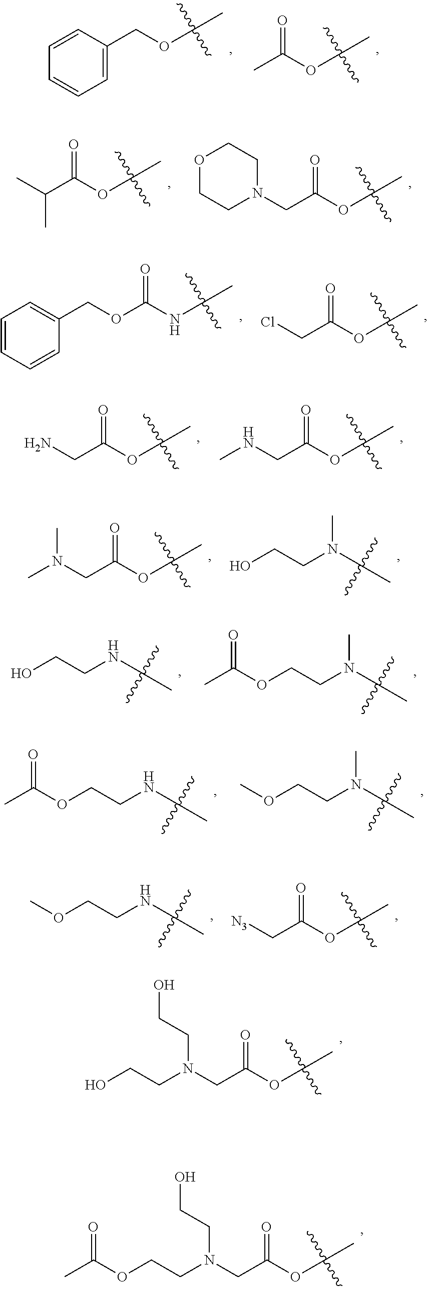

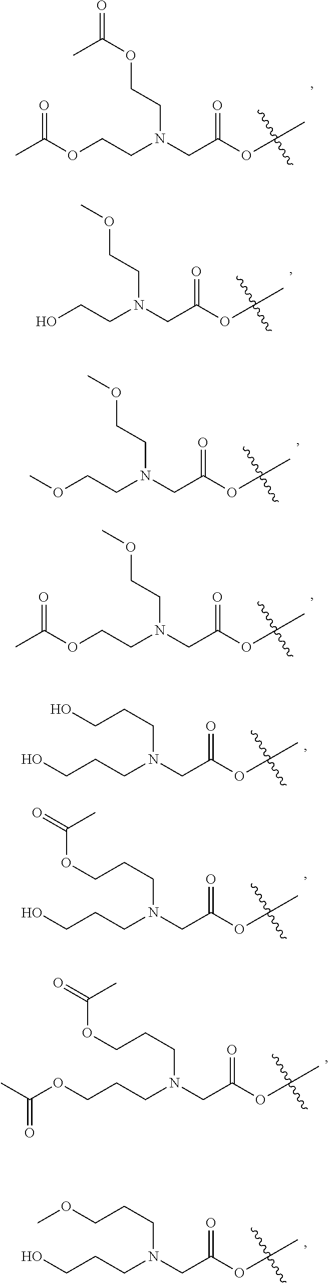

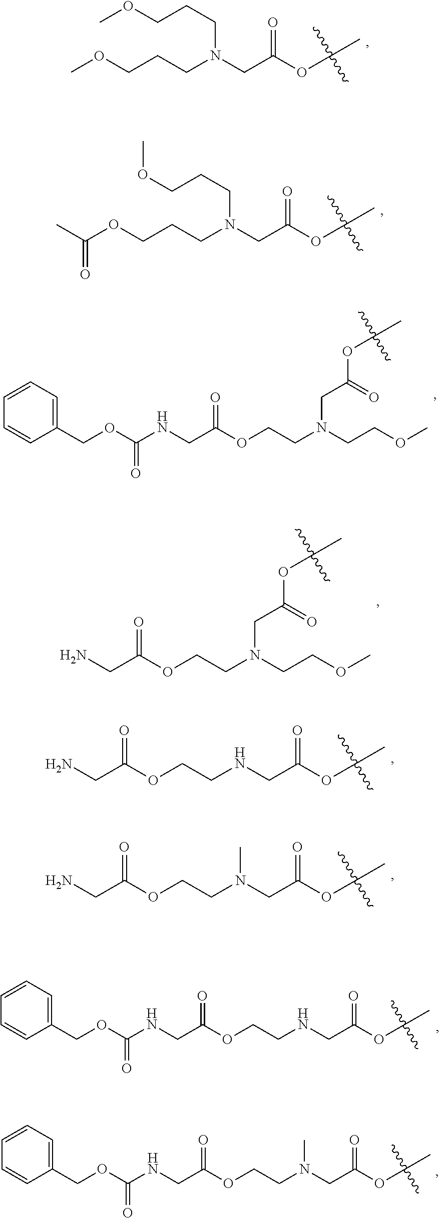

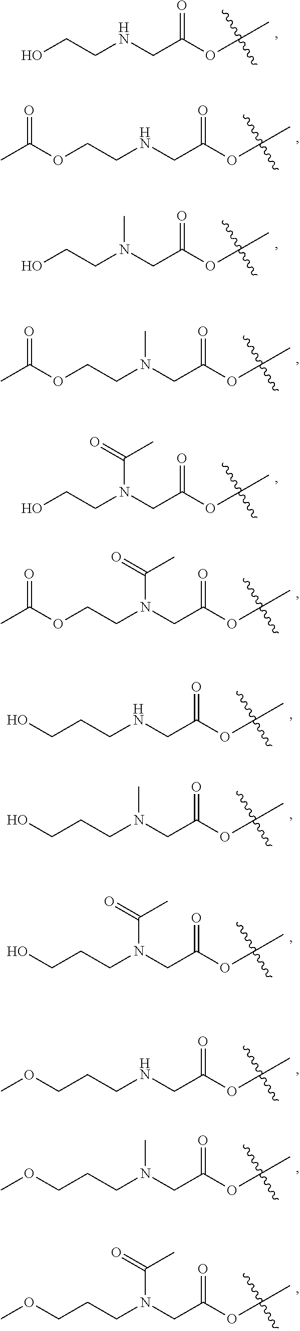

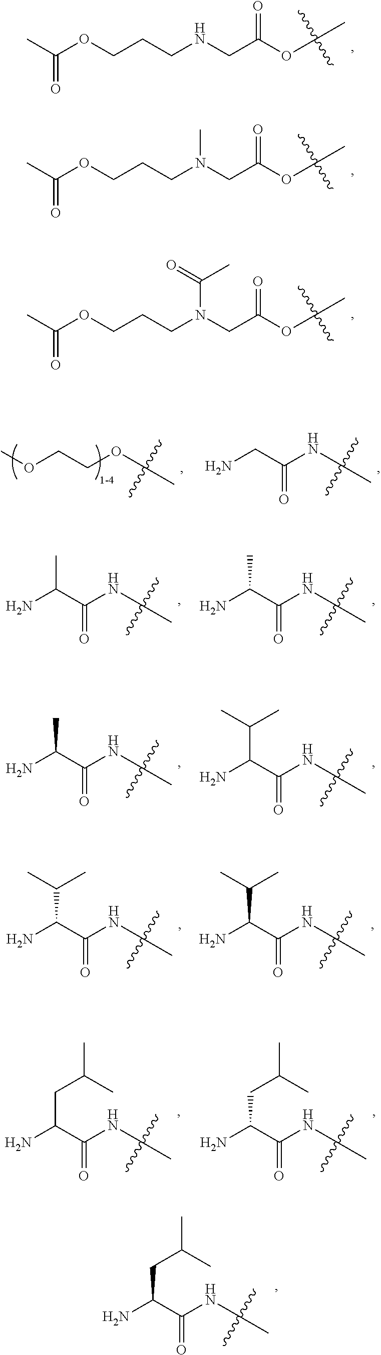

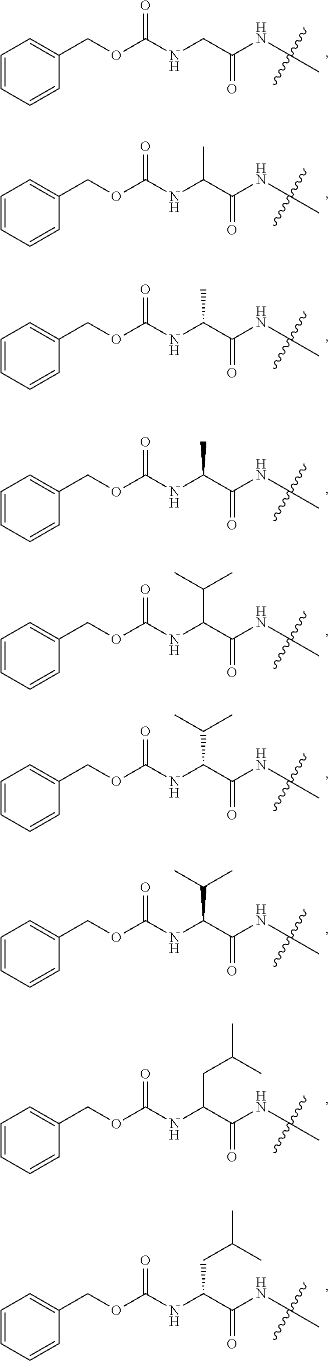

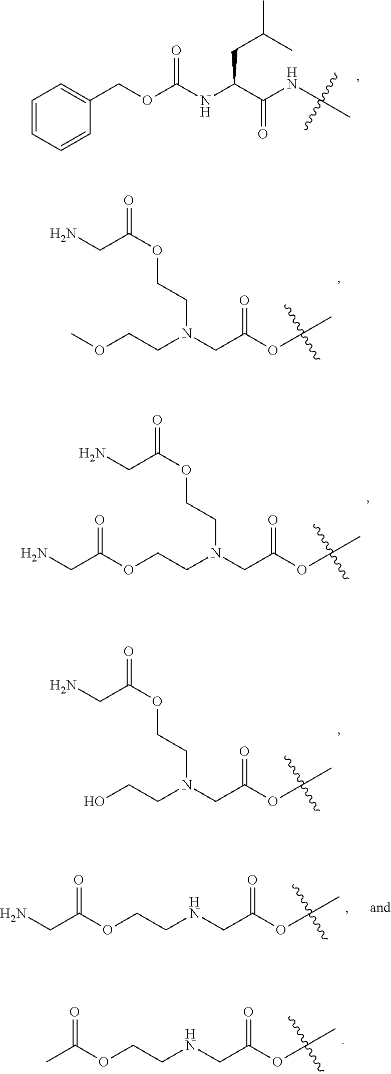 [SMILES: *NC(=O)[C@H](CC(C)C)NC(=O)OCc1ccccc1 ; *OC(=O)CN(CCOC)CCOC(=O)CN ; *OC(=O)CN(CCOC(=O)CN)CCOC(=O)CN ; *OC(=O)CN(CCO)CCOC(=O)CN ; *OC(=O)CNCCOC(=O)CN ; *OC(=O)CNCCOC(C)=O]

13. A compound of Formula (IA) 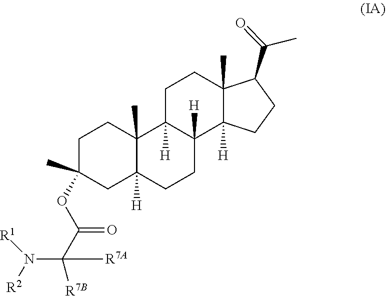
or a pharmaceutically acceptable salt, solvate, hydrate, tautomer, or stereoisomer thereof, wherein
R¹ and R² are each independently hydrogen, alkyl, alkenyl, alkynyl, heteroalkyl, cycloalkyl, heterocyclyl, aryl, heteroaryl, alkylene-cycloalkyl, alkylene-heterocyclyl, alkylene-aryl, alkylene-heteroaryl, heteroalkylene-cycloalkyl, heteroalkylene-heterocyclyl, heteroalkylene-aryl, heteroalkylene-heteroaryl, cycloalkylene-alkyl, cycloalkylene-heteroalkyl, heterocyclylene-alkyl, or heterocyclylene-heteroalkyl, wherein the alkyl, alkylene, alkenyl, alkynyl, heteroalkyl, heteroalkylene, cycloalkyl, cycloalkylene, heterocyclyl, heterocyclylene, aryl, or heteroaryl is optionally substituted;
or R¹ and R², together with the atoms to which they are attached, form a 3-7 membered cycloalkyl, heterocyclyl, aryl, or heteroaryl, wherein the cycloalkyl, heterocyclyl, aryl, or heteroaryl is optionally substituted; and
R^{7A} and R^{7B} are each independently hydrogen or optionally substituted alkyl.

14. The compound of claim 13, wherein the —N(R¹)(R²) group is a moiety selected from the group consisting of 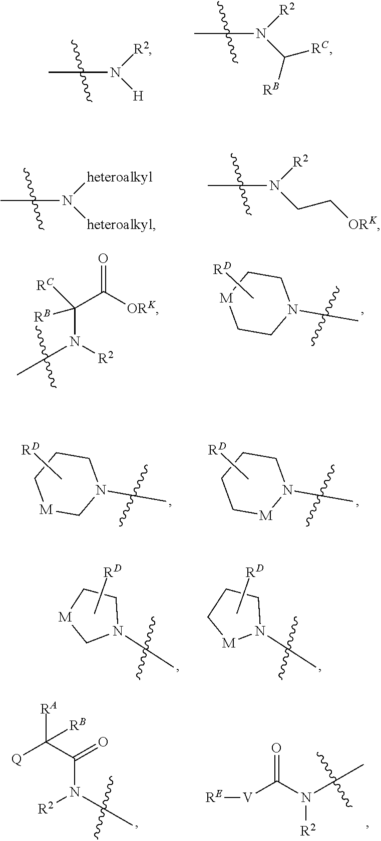
wherein R², M, Q, V, R^{A}, R^{B}, R^{C}, R^{D}, R^{E} and R^{K} are as described in claim 1, and [symbol] denotes the point of attachment of the nitrogen atom to the carbon atom of the —C(R^{7A})(R^{7B}) group.

15. The compound of claim 13, wherein the —N(R¹)(R²) group is a moiety selected from the group consisting of 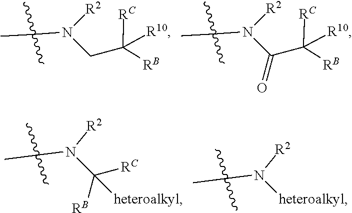
wherein R², R¹⁰, R^{B}, R^{C}, R^{D}, R^{E} and R^{K} are as described in claim 1, and [symbol] denotes the point of attachment of the nitrogen atom to the carbon atom of the —C(R^{7A})(R^{7B}) group.

16. The compound of any one of claims 1-6 or 13-15, wherein the X group of Formula (I), or the —N(R¹)(R²) group of Formula (IA), is a moiety selected from the group consisting of 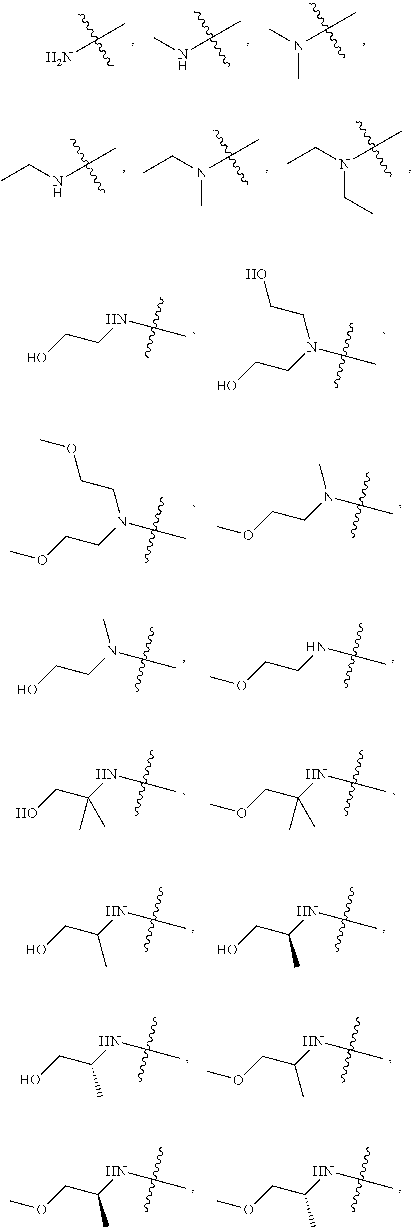 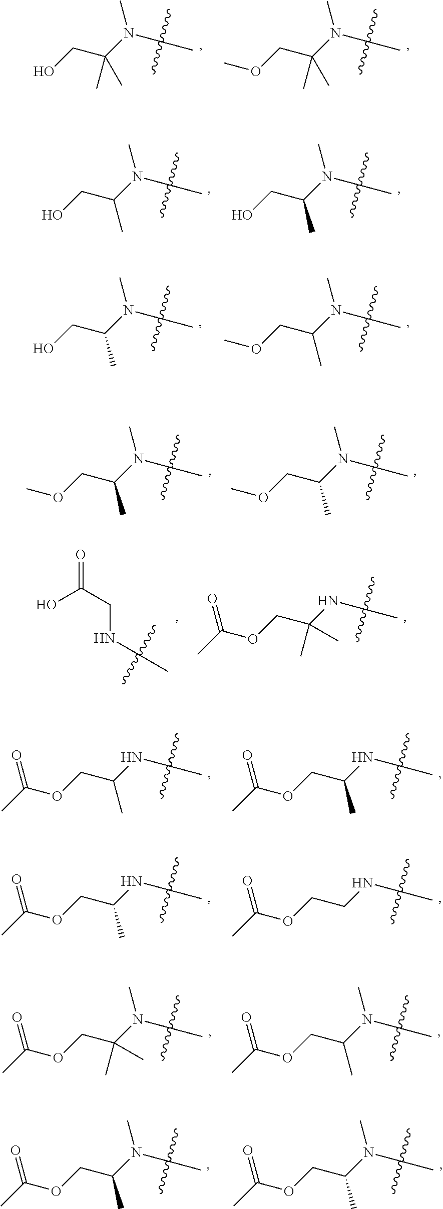 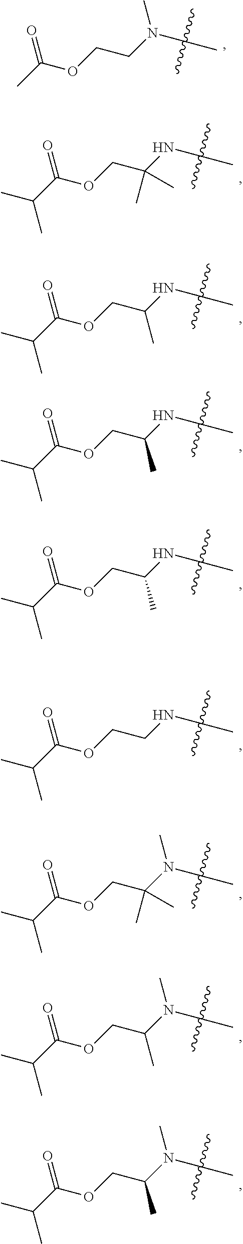 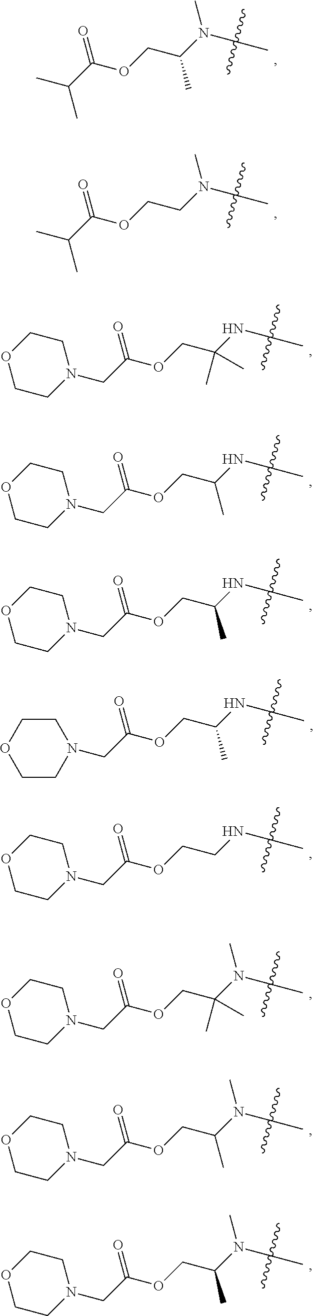 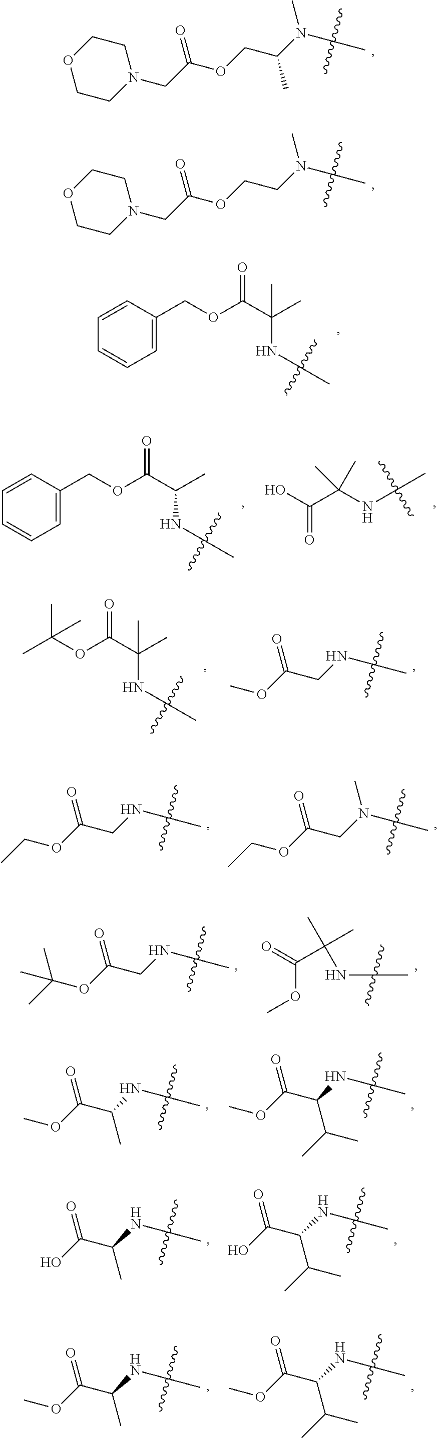 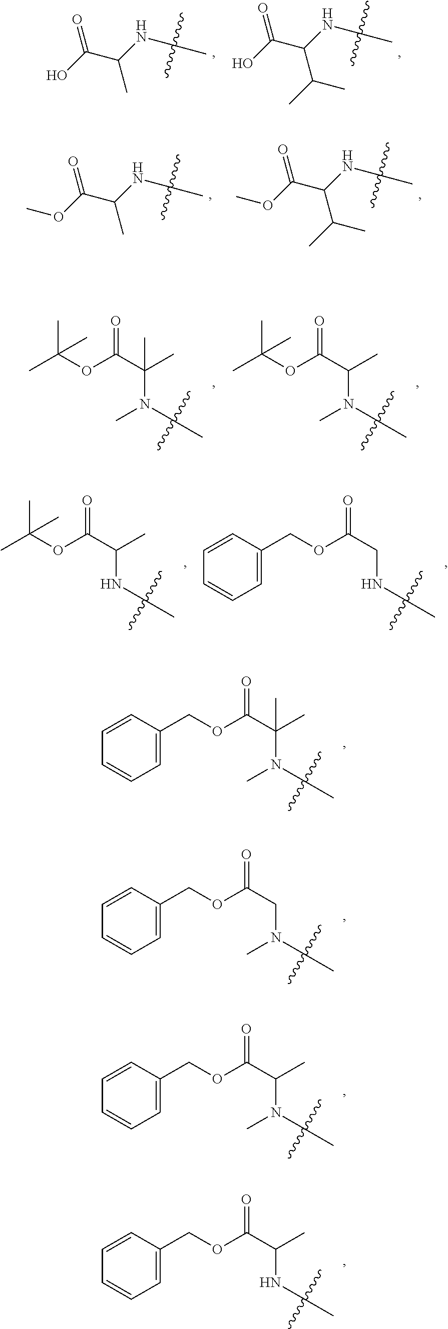 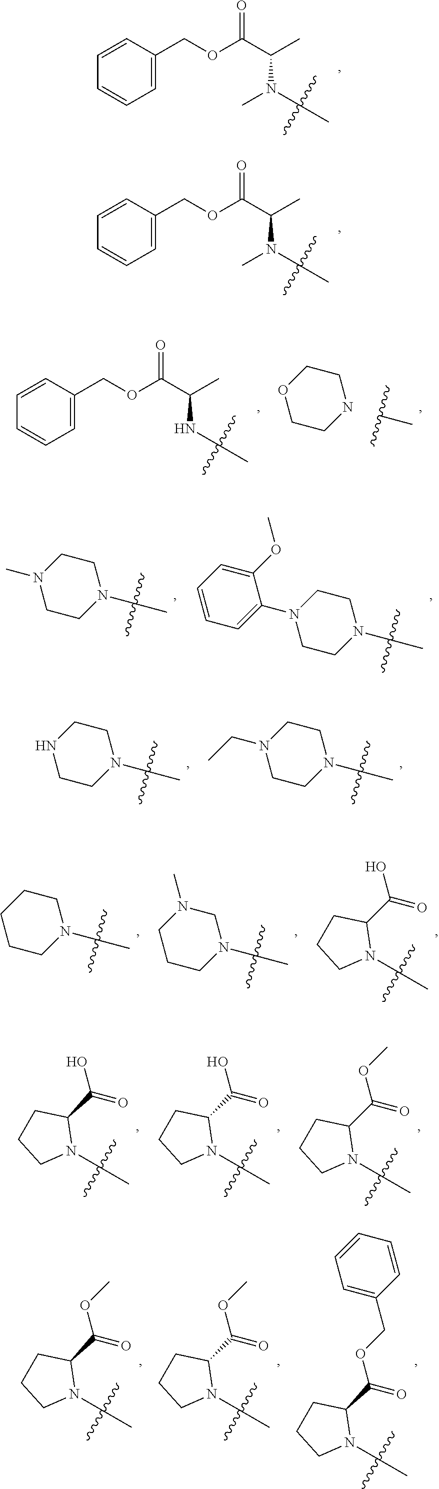 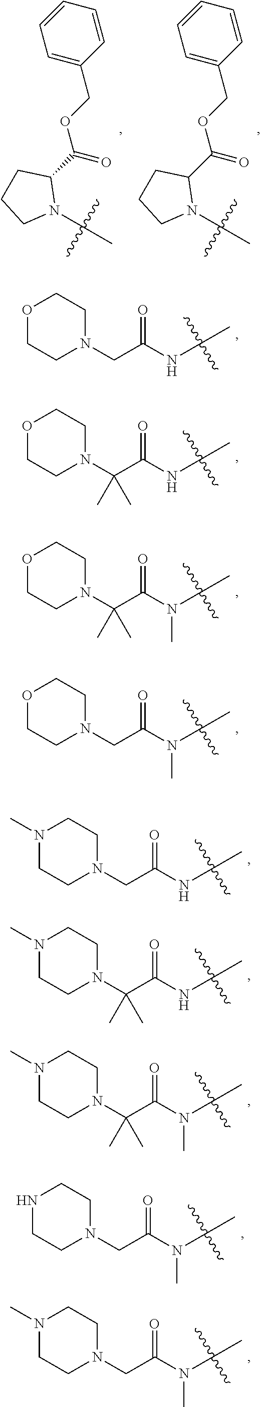 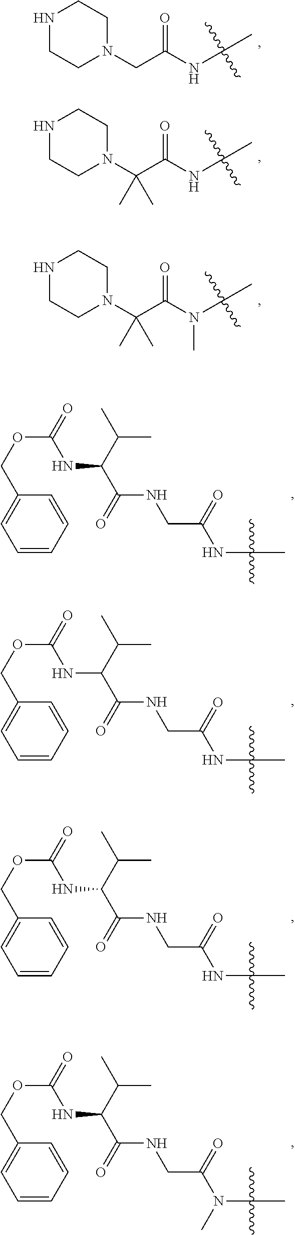 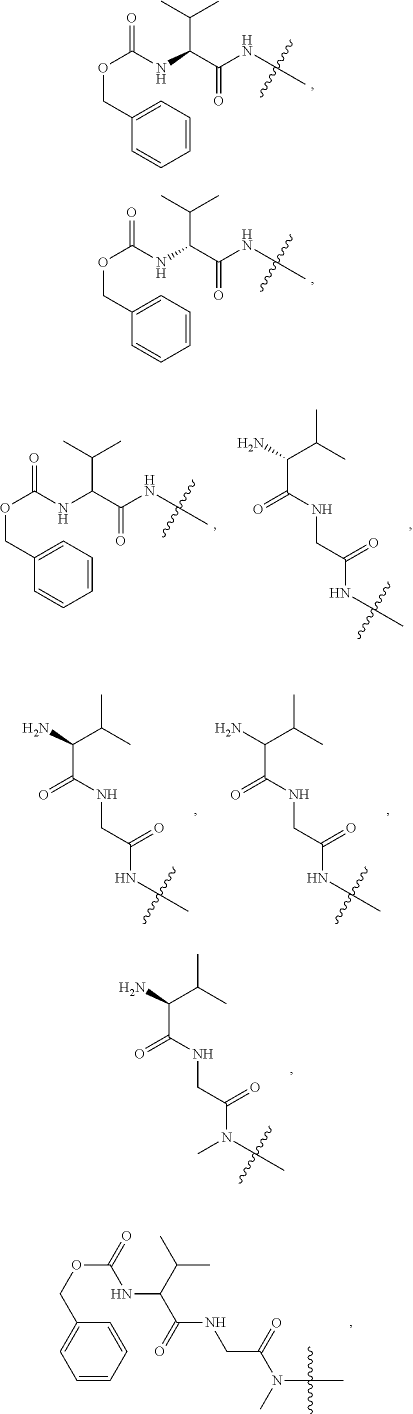 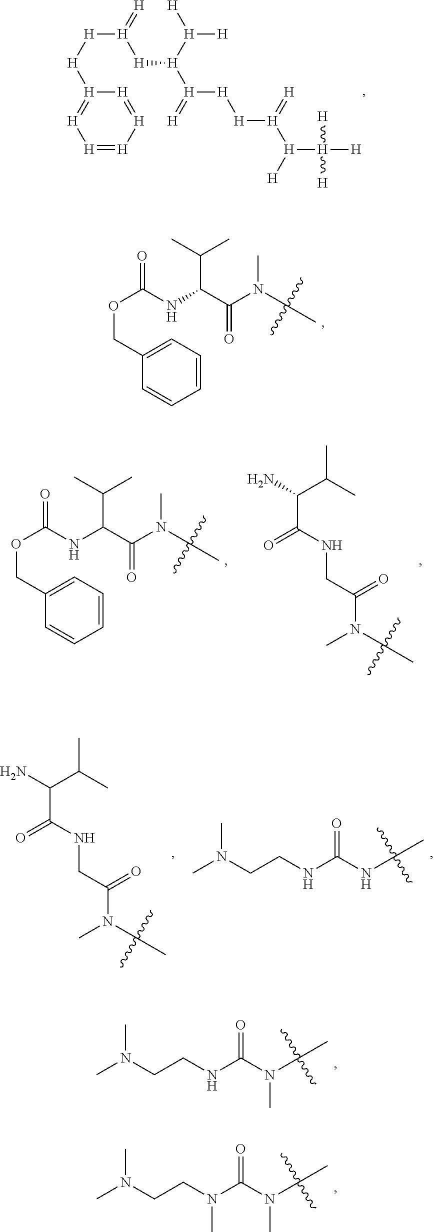 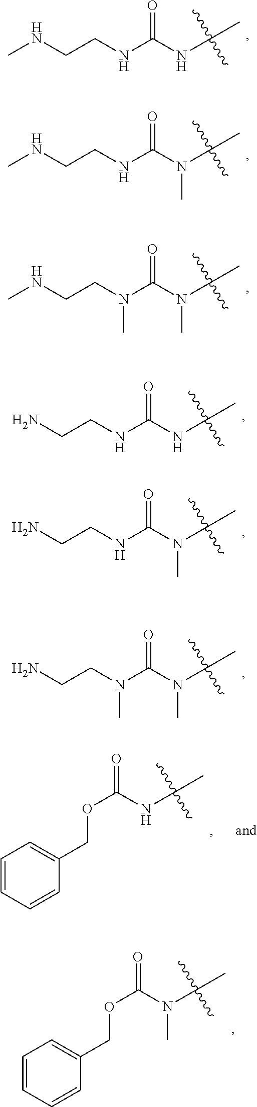
wherein [symbol] denotes the point of attachment of X to Z, or X to Y when Z is absent, in Formula (I); or the point of attachment of the nitrogen atom to the carbon atom of the —C(R^{7A})(R^{7B}) group in Formula (IA).

17. The compound of any one of claims 1-6 or 13-15, wherein the X group of Formula (I), or the —N(R¹)(R²) group of Formula (IA), is a moiety selected from the group consisting of 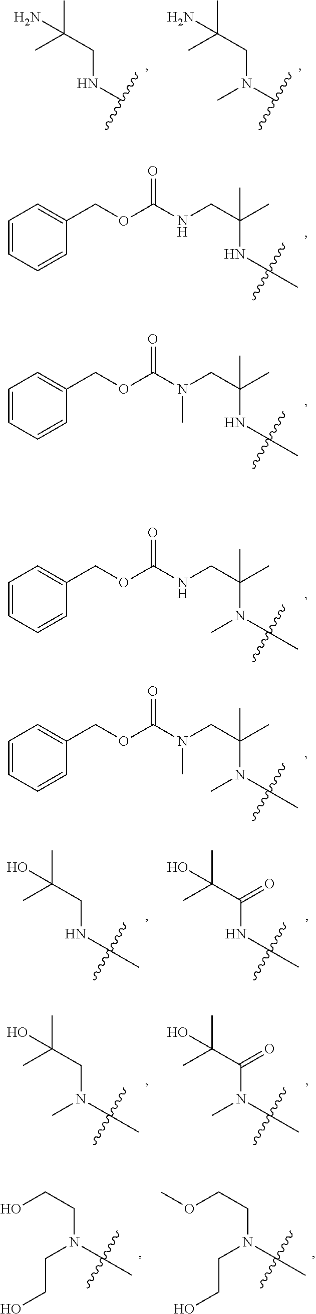 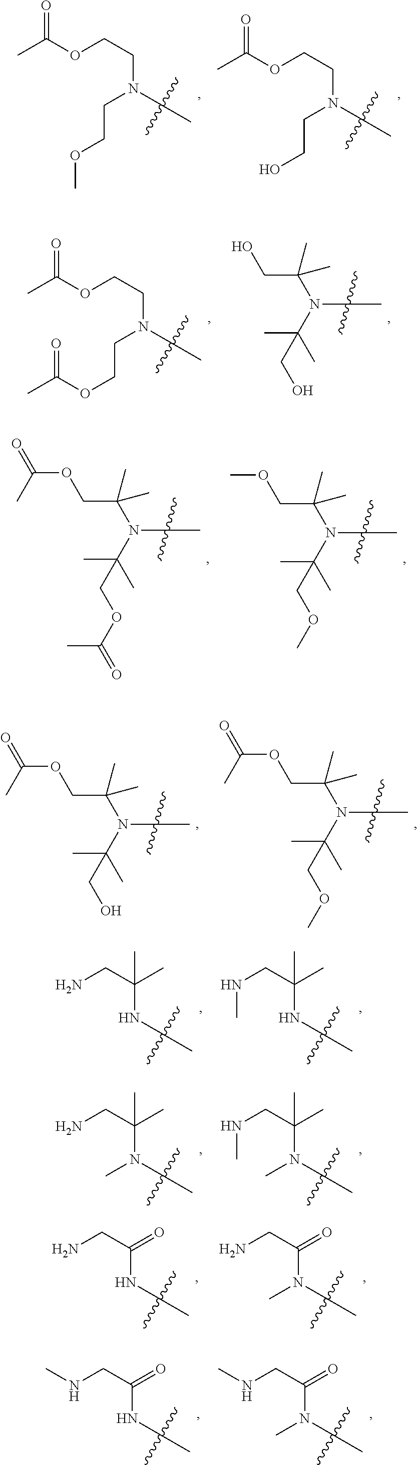 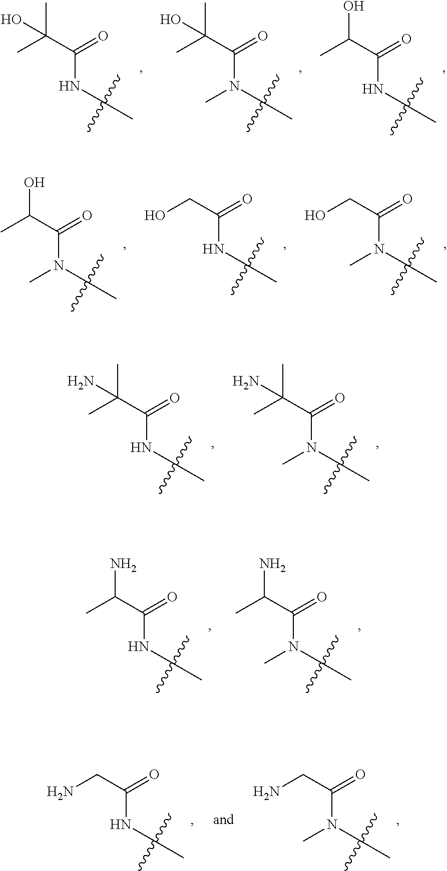
wherein [symbol] denotes the point of attachment of X to Z, or X to Y when Z is absent, in Formula (I); or the point of attachment of the nitrogen atom to the carbon atom of the —C(R^{7A})(R^{7B}) group in Formula (IA).

18. A compound of any one of the preceding claims, wherein the compound is a compound of Formula (IA-a) 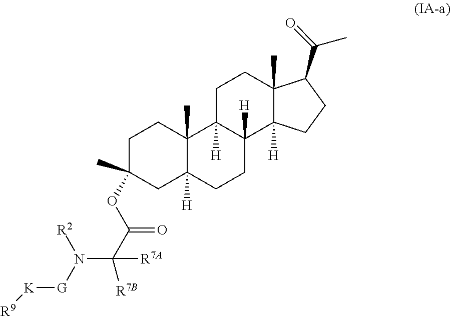
or a pharmaceutically acceptable salt, solvate, hydrate, tautomer, or stereoisomer thereof, wherein
G is absent, —C(O)—, or —C(R^{8A})(R^{8B})— (e.g., —CH₂—),
K is alkylene, heteroalkylene, —C(O)—, —OC(O)—, —C(O)O—, —O—, —O—(CH₂)—, or —(CH₂)—O—, wherein the alkylene or heteroalkylene is optionally substituted;
R², R^{7A}, R^{7B}, R^{8A}, and R^{8B} are each independently hydrogen or optionally substituted alkyl; and
R⁹ is hydrogen, alkyl, alkenyl, alkynyl, heteroalkyl, cycloalkyl, heterocyclyl, aryl, or heteroaryl, wherein the alkyl, alkenyl, alkynyl, heteroalkyl, cycloalkyl, heterocyclyl, aryl, or heteroaryl is optionally substituted.

19. The compound of claim 18, wherein
G is —C(R^{8A})(R^{8B})—;
K is —OC(O)—;
R², R^{7A}, R^{7B}, and R⁹ are each hydrogen; and
R^{8A} and R^{8B} are each independently hydrogen or alkyl (e.g., methyl), wherein the carbon atom of the carbonyl group in —OC(O)— is bonded to G.

20. The compound of any one of the preceding claims, wherein the compound is a compound of Formula (IA-b) 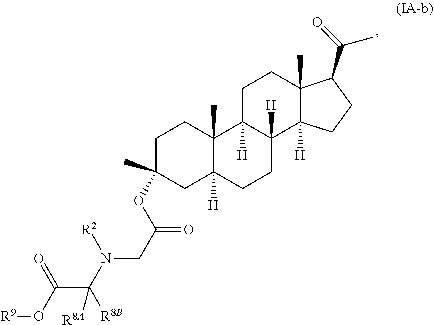
or a pharmaceutically acceptable salt, solvate, hydrate, tautomer, or stereoisomer thereof, wherein
R², R^{8A}, and R^{8B} are each independently hydrogen or optionally substituted alkyl; and
R⁹ is hydrogen, alkyl, alkenyl, alkynyl, heteroalkyl, cycloalkyl, heterocyclyl, aryl, or heteroaryl, wherein the alkyl, alkenyl, alkynyl, heteroalkyl, cycloalkyl, heterocyclyl, aryl, or heteroaryl is optionally substituted.

21. The compound of any one of claims 18-20, wherein
R² is hydrogen; and
R^{8A} and R^{8B} are each independently methyl.

22. A compound of Formula (IB): 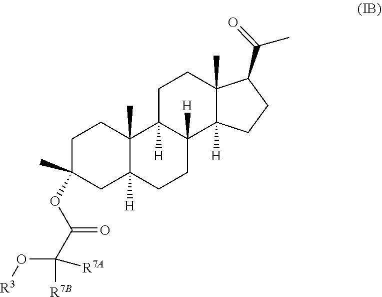
or a pharmaceutically acceptable salt, solvate, hydrate, tautomer, or stereoisomer thereof, wherein
R³ is hydrogen, alkyl, alkenyl, alkynyl, heteroalkyl, cycloalkyl, heterocyclyl, aryl, heteroaryl, alkylene-cycloalkyl, alkylene-heterocyclyl, alkylene-aryl, alkylene-heteroaryl, heteroalkylene-cycloalkyl, heteroalkylene-heterocyclyl, heteroalkylene-aryl, heteroalkylene-heteroaryl, cycloalkylene-alkyl, cycloalkylene-heteroalkyl, heterocyclylene-alkyl, or heterocyclylene-heteroalkyl, wherein the alkyl, alkylene, alkenyl, alkynyl, heteroalkyl, heteroalkylene, cycloalkyl, cycloalkylene, heterocyclyl, heterocyclylene, aryl, or heteroaryl is optionally substituted; and
R^{7A} and R^{7B} are each independently hydrogen or optionally substituted alkyl.

23. The compound of claim 22, wherein the —O—(R³) group is a moiety selected from the group consisting of 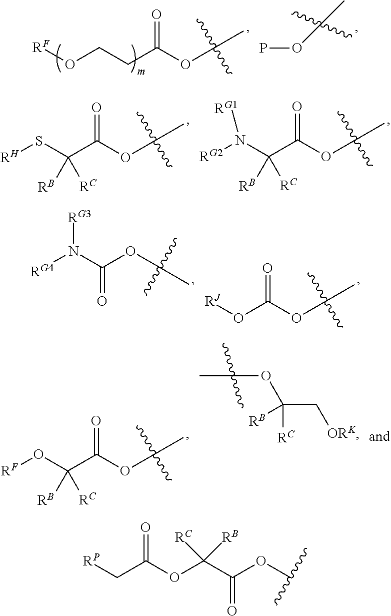
wherein
P is an amino acid residue (e.g., glycine, alanine, valine, leucine, or proline), that may be a D-amino acid residue or L-amino acid residue, and is optionally substituted (e.g., at a terminal nitrogen atom, e.g., by a carbonate group, e.g., a carboxybenzyl (Cbz) group),
or P is a dipeptide moiety (e.g., a leucine-leucine moiety) that may comprise D-amino acids, L-amino acids, or a combination thereof, and is optionally substituted (e.g., at one or more nitrogen atoms, e.g., at a terminal nitrogen atom, e.g., by a carbonate group, e.g., a carboxybenzyl (Cbz) group);
R^{B} and R^{C} are each independently hydrogen, alkyl, heteroalkyl, cycloalkyl, heterocyclyl, aryl, or heteroaryl, wherein the alkyl, heteroalkyl, cycloalkyl, heterocyclyl, aryl, or heteroaryl is optionally substituted;
R^{F} and R^{H} are each independently hydrogen or alkyl;
R^{G1} and R^{G2} are each independently hydrogen, alkyl, or an amino acid residue (e.g., glycine, alanine, valine, leucine or proline), that may be a D-amino acid residue or L-amino acid residue, and is optionally substituted (e.g., at a terminal nitrogen atom, e.g., by a carbonate group, e.g., a carboxybenzyl (Cbz) group),
or R^{G1} and R^{G2}, together with the atoms to which they are attached, form a 3-7 membered cycloalkyl, heterocyclyl, aryl, or heteroaryl, wherein the cycloalkyl, heterocyclyl, aryl, or heteroaryl is optionally substituted;
R^{G3} and R^{G4} are each independently hydrogen, alkyl, or heteroalkyl, wherein the alkyl or heteroalkyl is optionally substituted;
or R^{G3} and R^{G4}, together with the atoms to which they are attached, form a 3-7 membered cycloalkyl, heterocyclyl, aryl, or heteroaryl, wherein the cycloalkyl, heterocyclyl, aryl, or heteroaryl is optionally substituted;
R^{J} is hydrogen, alkyl, alkylene-heterocyclyl, heteroalkyl, or heterocyclyl, wherein the alkyl, alkylene, heteroalkyl, or heterocyclyl is optionally substituted;
R^{K} is hydrogen, alkyl, heteroalkyl, cycloalkyl, heterocyclyl, aryl, or heteroaryl, wherein the alkyl, heteroalkyl, cycloalkyl, heterocyclyl, aryl, or heteroaryl is optionally substituted;
R^{P} is halo, —NH-alkyl, —NH-heteroalkyl, —N-(alkyl)₂ (e.g., —N(Me)₂), —N-(heteroalkyl)₂ (e.g., —N(CH₂CH₂OH)₂), or heterocyclyl (e.g., morpholinyl);
m is an integer of 1-5; and [symbol]
denotes the point of attachment of the oxygen atom to the carbon atom of the —C(R^{7A})(R^{7B}) group.

24. The compound of claim 22, wherein the —O—(R³) group is a moiety selected from the group consisting of 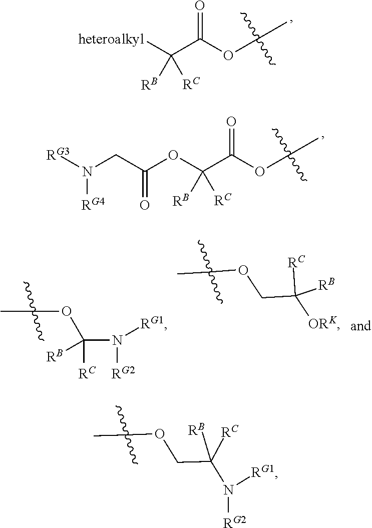
wherein
R^{B} and R^{C} are each independently hydrogen, alkyl, heteroalkyl, cycloalkyl, heterocyclyl, aryl, or heteroaryl, wherein the alkyl, heteroalkyl, cycloalkyl, heterocyclyl, aryl, or heteroaryl is optionally substituted;
R^{K} is hydrogen, alkyl, heteroalkyl, cycloalkyl, heterocyclyl, aryl, or heteroaryl, wherein the alkyl, heteroalkyl, cycloalkyl, heterocyclyl, aryl, or heteroaryl is optionally substituted;
R^{G1} and R^{G2} are each independently hydrogen, alkyl, or an amino acid residue (e.g., glycine, alanine, valine, leucine or proline), that may be a D-amino acid residue or L-amino acid residue, and is optionally substituted (e.g., at a terminal nitrogen atom, e.g., by a carbonate group, e.g., a carboxybenzyl (Cbz) group),
or R^{G1} and R^{G2}, together with the atoms to which they are attached, form a 3-7 membered cycloalkyl, heterocyclyl, aryl, or heteroaryl, wherein the cycloalkyl, heterocyclyl, aryl, or heteroaryl is optionally substituted; and
R^{G3} and R^{G4} are each independently hydrogen, alkyl, or heteroalkyl, wherein the alkyl or heteroalkyl is optionally substituted;
or R^{G3} and R^{G4}, together with the atoms to which they are attached, form a 3-7 membered cycloalkyl, heterocyclyl, aryl, or heteroaryl, wherein the cycloalkyl, heterocyclyl, aryl, or heteroaryl is optionally substituted.

25. The compound of any one of claims claim 1, 2, or 22-24, wherein the X group of Formula (I), or the —O—R³ group of Formula (IB), is a moiety selected from the group consisting of 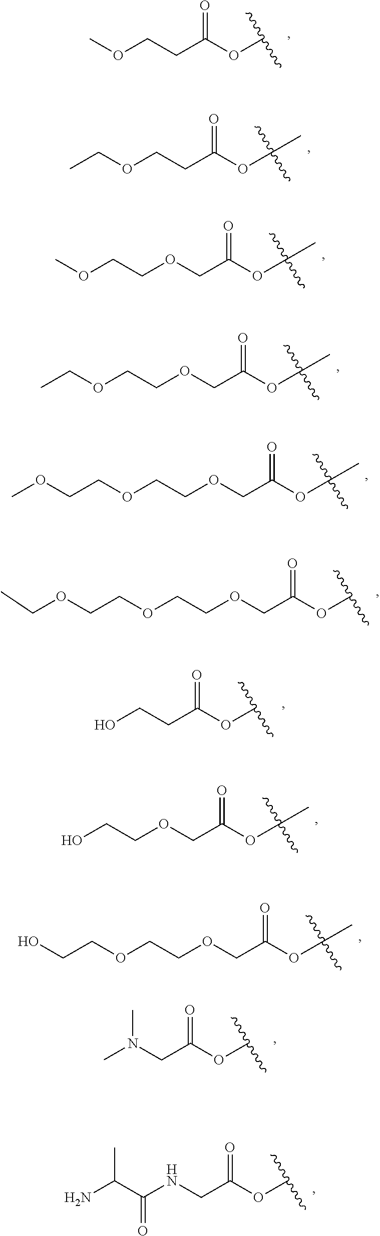 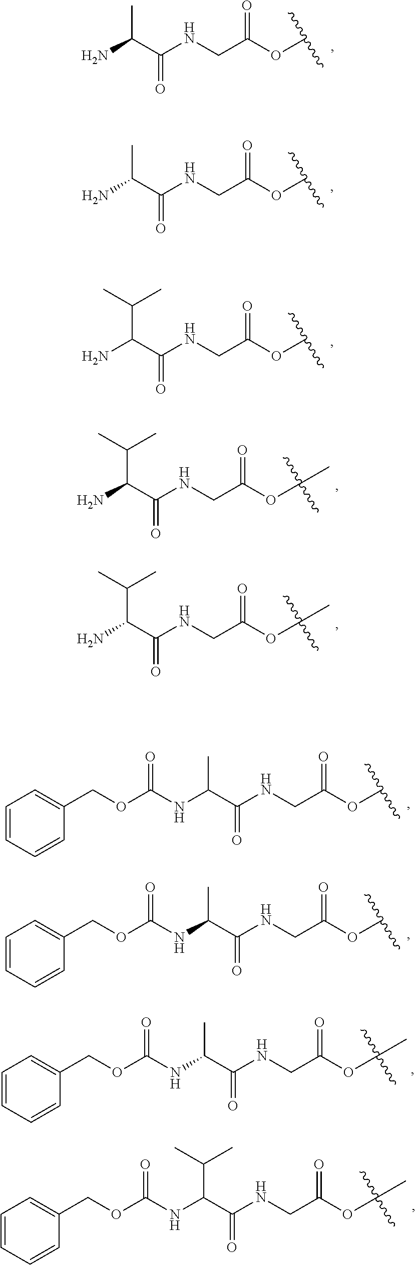 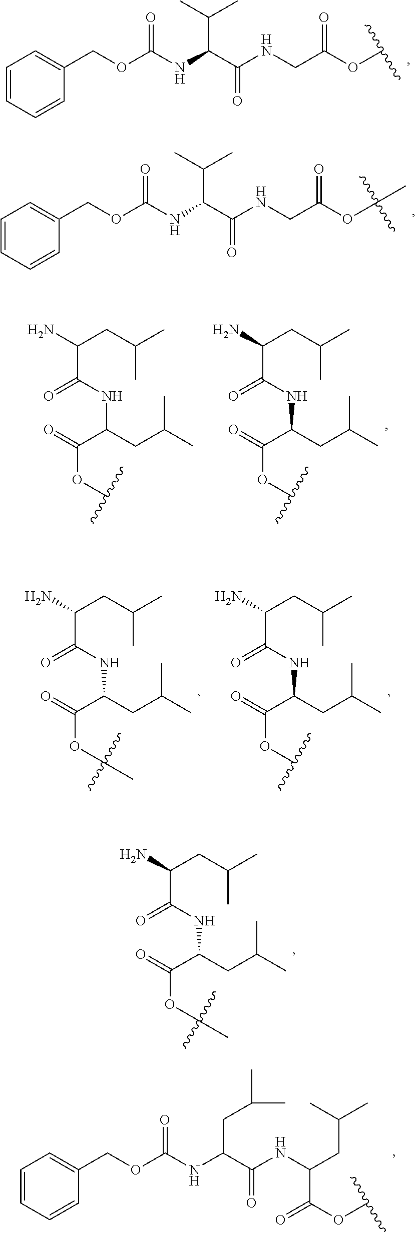 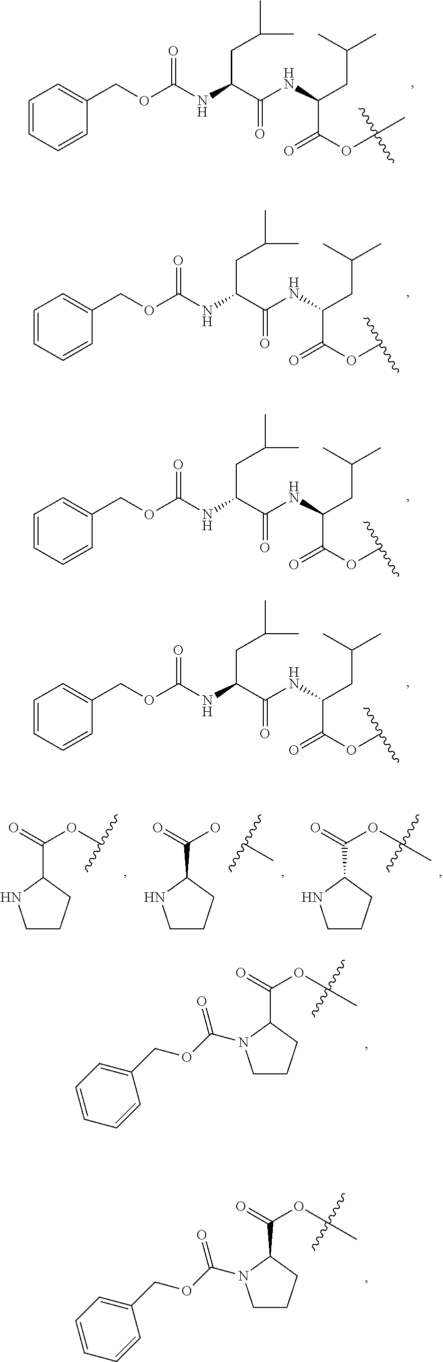 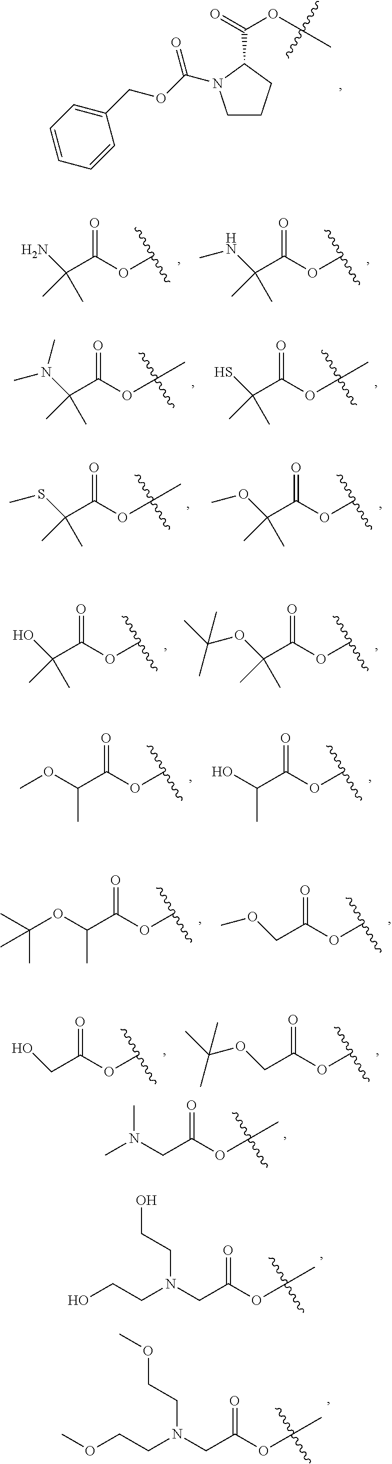 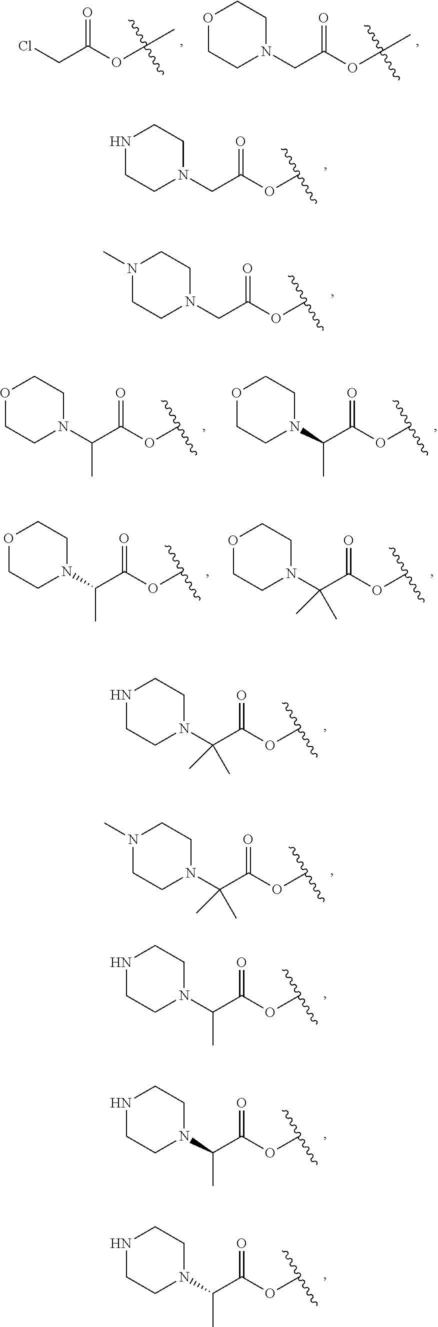 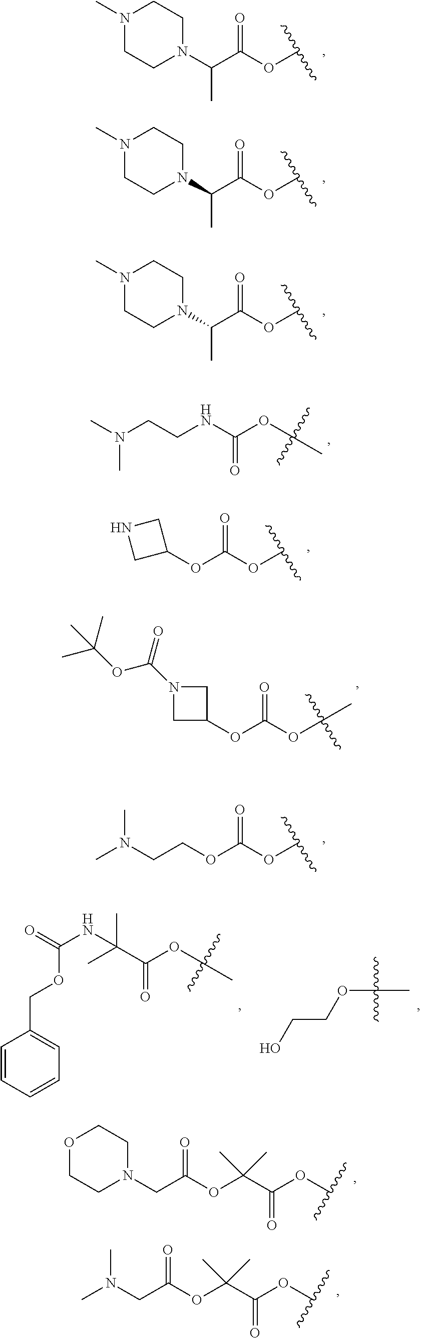 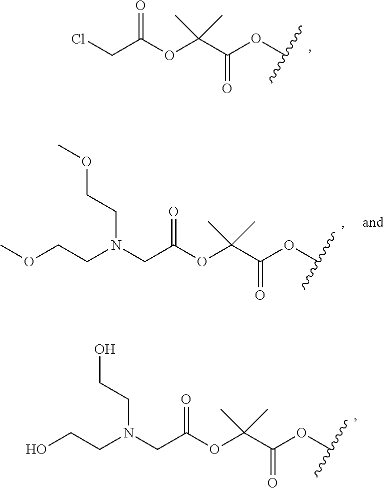
wherein [symbol] denotes the point of attachment of X to Z, or X to Y when Z is absent, in Formula (I); or the point of attachment of the oxygen atom to the carbon atom of the —C(R^{7A})(R^{7B}) group in Formula (IB).

26. The compound of any one of claims claim 1, 2, or 22-24, wherein the X group of Formula (I), or the —O—R³ group of Formula (IB), is a moiety selected from the group consisting of 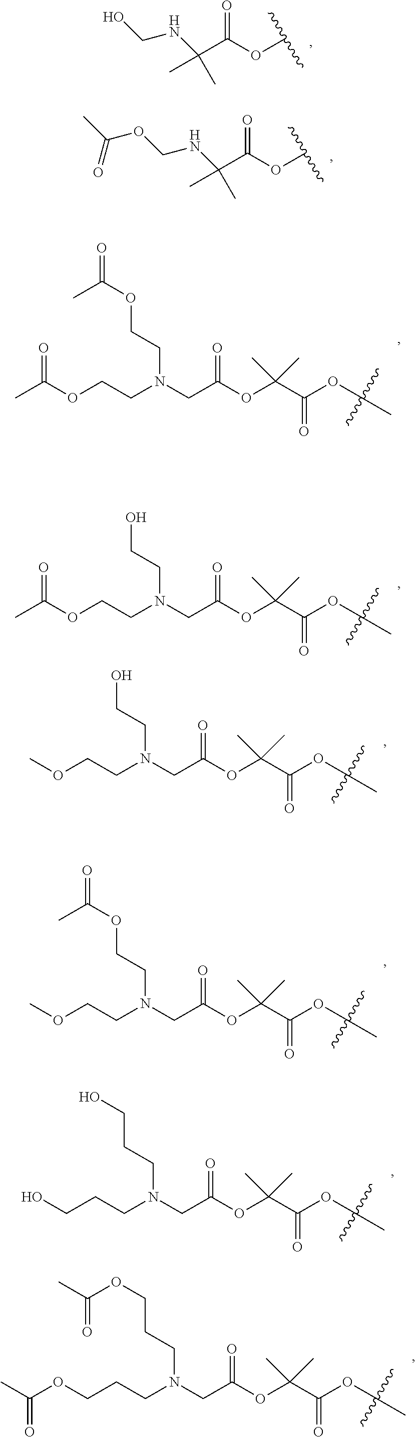 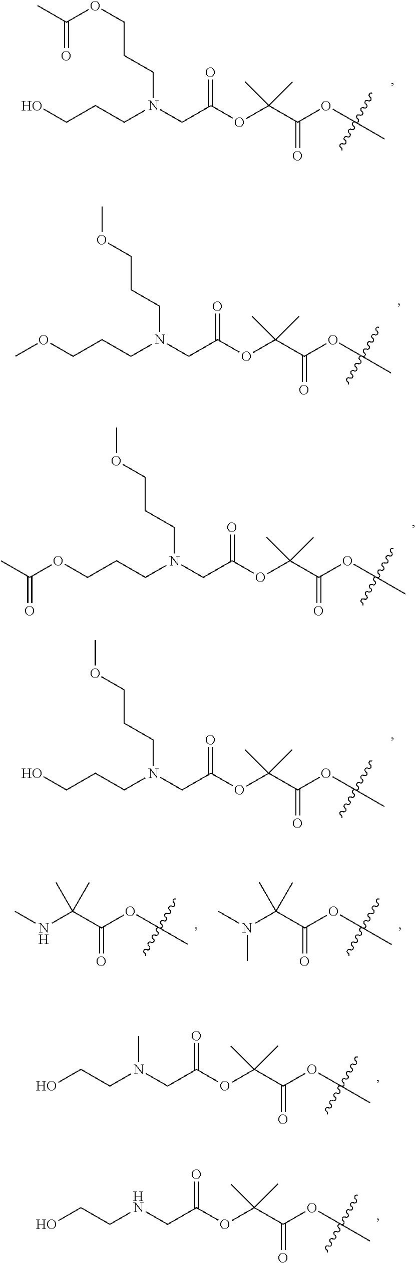 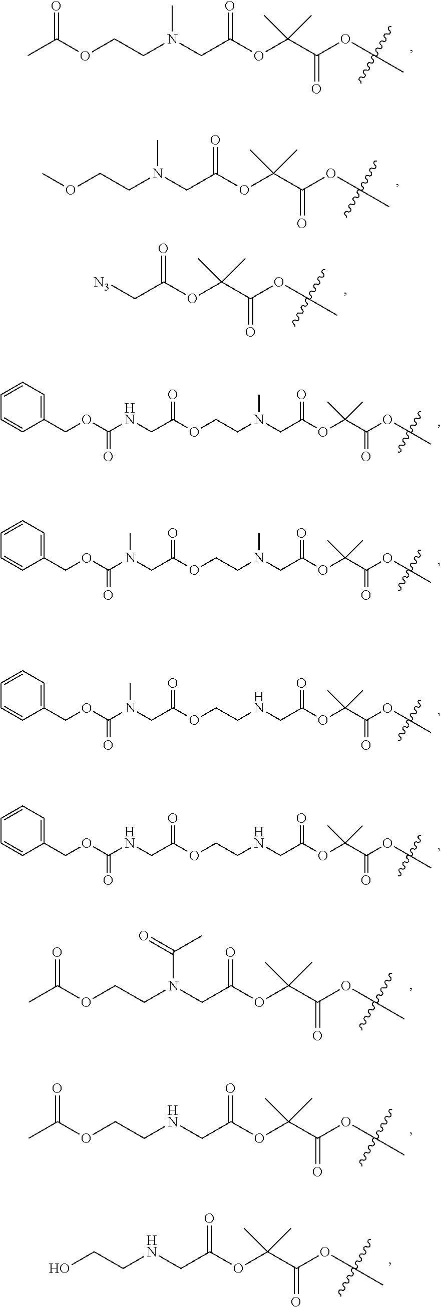 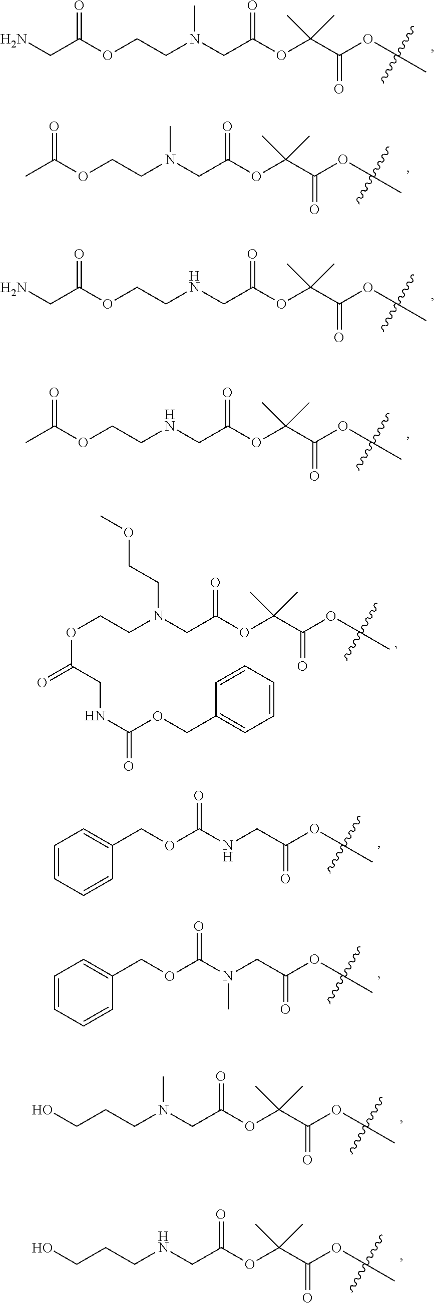 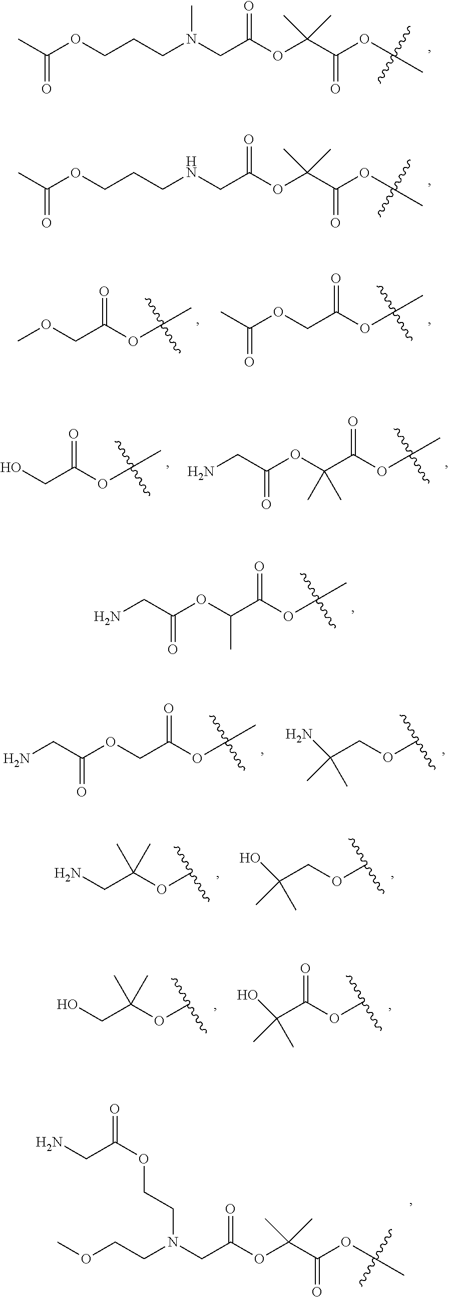 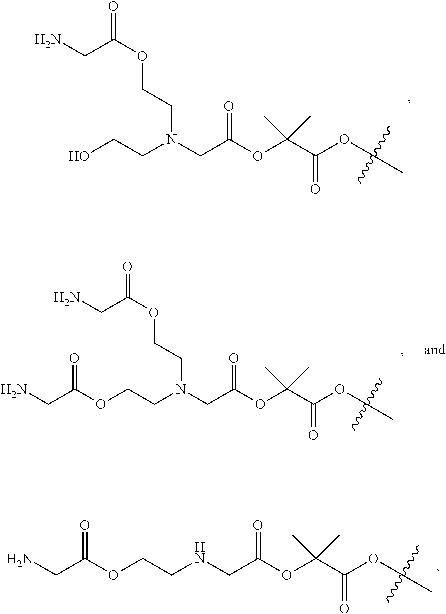
wherein [symbol] denotes the point of attachment of X to Z, or X to Y when Z is absent, in Formula (I); or the point of attachment of the oxygen atom to the carbon atom of the —C(R^{7A})(R^{7B}) group in Formula (IB).

27. A compound of Formula (IC) 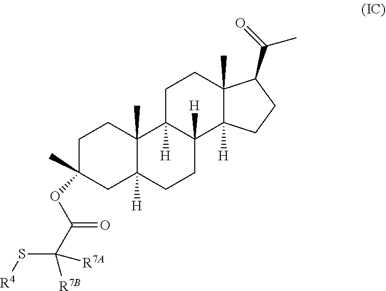
or a pharmaceutically acceptable salt, solvate, hydrate, tautomer, or stereoisomer thereof, wherein
R⁴ is hydrogen, alkyl, alkenyl, alkynyl, heteroalkyl, cycloalkyl, heterocyclyl, aryl, heteroaryl, alkylene-cycloalkyl, alkylene-heterocyclyl, alkylene-aryl, alkylene-heteroaryl, heteroalkylene-cycloalkyl, heteroalkylene-heterocyclyl, heteroalkylene-aryl, or heteroalkylene-heteroaryl, wherein the alkyl, alkylene, alkenyl, alkynyl, heteroalkyl, heteroalkylene, cycloalkyl, cycloalkylene, heterocyclyl, heterocyclylene, aryl, or heteroaryl is optionally substituted; and
R^{7A} and R^{7B} are each independently hydrogen or optionally substituted alkyl.

28. The compound of claim 27, wherein the-S—(R⁴) group is a moiety selected from the group consisting of 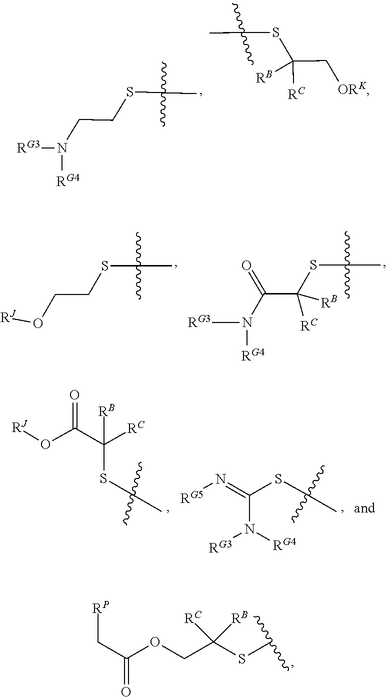
wherein
R^{B} and R^{C} are each independently hydrogen, alkyl, heteroalkyl, cycloalkyl, heterocyclyl, aryl, or heteroaryl, wherein the alkyl, heteroalkyl, cycloalkyl, heterocyclyl, aryl, or heteroaryl is optionally substituted;
R^{G3} and R^{G4} are each independently hydrogen, alkyl, or heteroalkyl, wherein the alkyl or heteroalkyl is optionally substituted;
or R^{G3} and R^{G4}, together with the atoms to which they are attached, form a 3-7 membered cycloalkyl, heterocyclyl, aryl, or heteroaryl, wherein the cycloalkyl, heterocyclyl, aryl, or heteroaryl is optionally substituted;
R^{G5} is hydrogen, alkyl, or heteroalkyl, wherein the alkyl or heteroalkyl is optionally substituted;
R is hydrogen, alkyl, alkylene-heterocyclyl, heteroalkyl, or heterocyclyl, wherein the alkyl, alkylene, heteroalkyl, or heterocyclyl is optionally substituted;
R^{K} is hydrogen, alkyl, heteroalkyl, cycloalkyl, heterocyclyl, aryl, or heteroaryl, wherein the alkyl, heteroalkyl, cycloalkyl, heterocyclyl, aryl, or heteroaryl is optionally substituted;
R^{P} is halo, —NH-alkyl, —NH-heteroalkyl, —N-(alkyl)₂ (e.g., —N(Me)₂), —N-(heteroalkyl)₂ (e.g., —N(CH₂CH₂OH)₂), or heterocyclyl (e.g., morpholinyl); and [symbol]
denotes the point of attachment of the sulfur atom to the carbon atom of the —C(R^{7A})(R^{7B}) group.

29. The compound of any one of claims 1, 2, 27 or 28, wherein the X group of Formula (I), or the —S—R⁴ group of Formula IC, is a moiety selected from the group consisting of 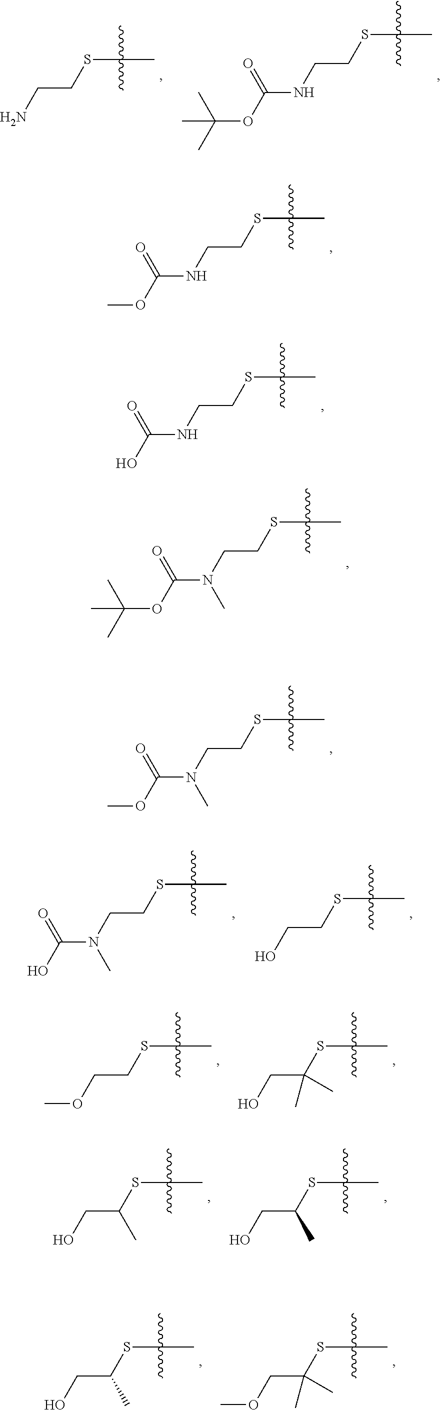 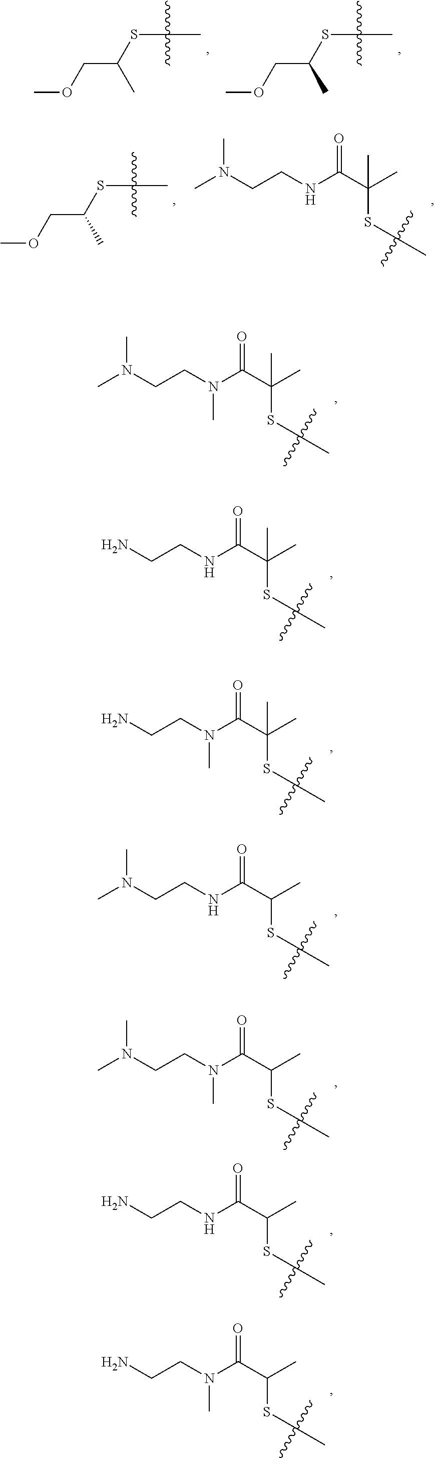 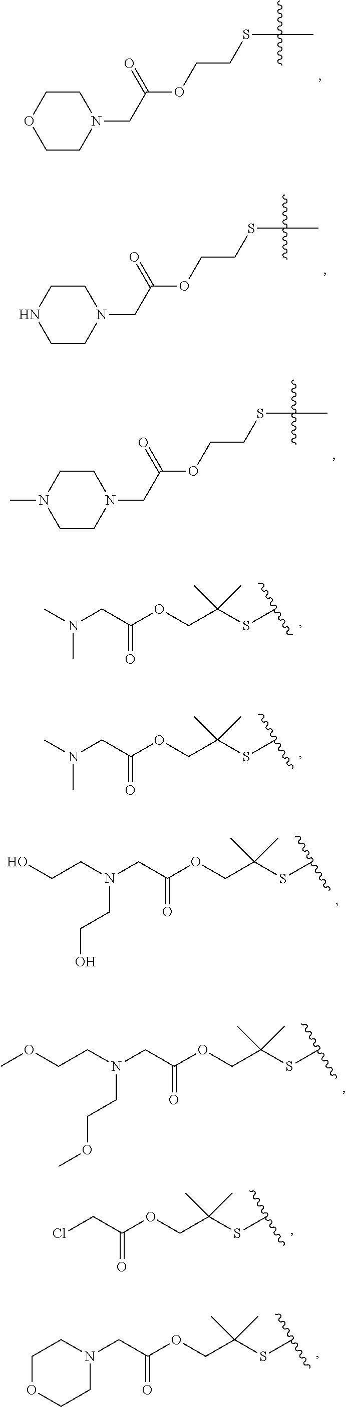 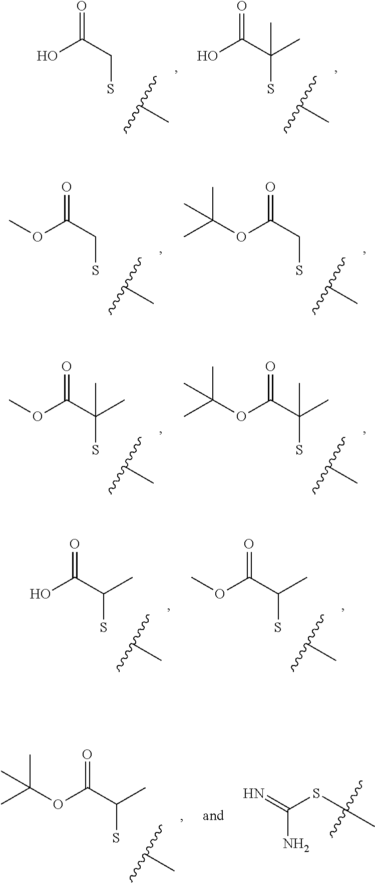
wherein [symbol] denotes the point of attachment of X to Z, or X to Y when Z is absent, in Formula (I); or the point of attachment of the sulfur atom to the carbon atom of the —C(R^{7A})(R^{7B}) group in Formula (IC).

30. A compound of Formula (ID) 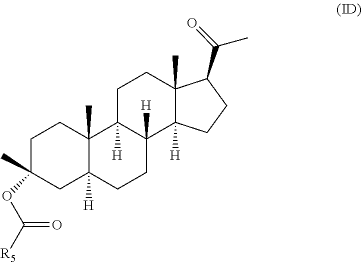
or a pharmaceutically acceptable salt, solvate, hydrate, tautomer, or stereoisomer thereof, wherein
R⁵ is alkyl, alkenyl, alkynyl, heteroalkyl, haloalkyl, cycloalkyl, heterocyclyl, aryl, heteroaryl, haloalkyl, alkylene-heterocyclyl, alkylene-aryl, alkylene-heteroaryl, heteroalkylene-heterocyclyl, heteroalkylene-aryl, heteroalkylene-heteroaryl, cycloalkylene-alkyl, cycloalkylene-heteroalkyl, heterocyclylene-alkyl, or heterocyclylene-heteroalkyl, wherein the alkyl, alkylene, alkenyl, alkynyl, heteroalkyl, heteroalkylene, cycloalkyl, cycloalkylene, heterocyclyl, heterocyclylene, aryl, or heteroaryl is optionally substituted.

31. The compound of claim 30, wherein R⁵ comprises a moiety selected from group consisting of 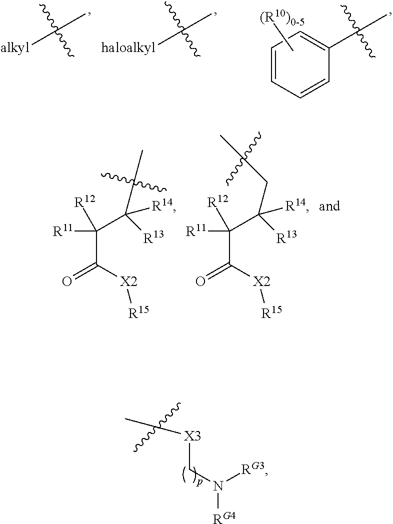
wherein
X2 and X3 are each independently —CH₂—, —O—, —S—, or —N(R^{G3})— (e.g., —NH—);
R¹⁰ is alkyl, alkenyl, alkynyl, haloalkyl, halo, —OR, —N(R^{G3})(R^{G4}); —NO₂, —CN, or —NC;
R¹¹, R¹², R¹³, and R¹⁴ are each independently hydrogen, alkyl, alkenyl, alkynyl, heteroalkyl, haloalkyl, halo, or —N(R^{G3})(R^{G4}), wherein the alkyl, alkenyl, alkynyl, heteroalkyl, or haloalkyl is optionally substituted,
or one of R¹¹ and R¹², and one of R¹³ and R¹⁴, together with the atoms to which they are attached, form a 3-7 membered cycloalkyl, heterocyclyl, aryl, or heteroaryl, wherein the cycloalkyl, heterocyclyl, aryl, or heteroaryl is optionally substituted;
R¹⁵ is hydrogen, alkyl, alkenyl, alkynyl, heteroalkyl, haloalkyl, or halo; wherein the alkyl, alkenyl, alkynyl, heteroalkyl, or haloalkyl is optionally substituted;
R^{G3} and R^{G4} are each independently hydrogen, alkyl, or heteroalkyl, wherein the alkyl or heteroalkyl is optionally substituted;
or R^{G3} and R^{G4}, together with the atoms to which they are attached, form a 3-7 membered cycloalkyl, heterocyclyl, aryl, or heteroaryl, wherein the cycloalkyl, heterocyclyl, aryl, or heteroaryl is optionally substituted;
R^{J} is hydrogen, alkyl, alkylene-heterocyclyl, heteroalkyl, or heterocyclyl, wherein the alkyl, alkylene, heteroalkyl, or heterocyclyl is optionally substituted;
and
p is an integer between 0 and 4; and [symbol]
denotes the point of attachment of the R⁵ group to the carbon atom of the —C(O)— group.

32. The compound of any one of claims 1, 2, 30, or 31, wherein the X group of Formula (I), or the R⁵ group of Formula (ID), is a moiety selected from the group consisting of 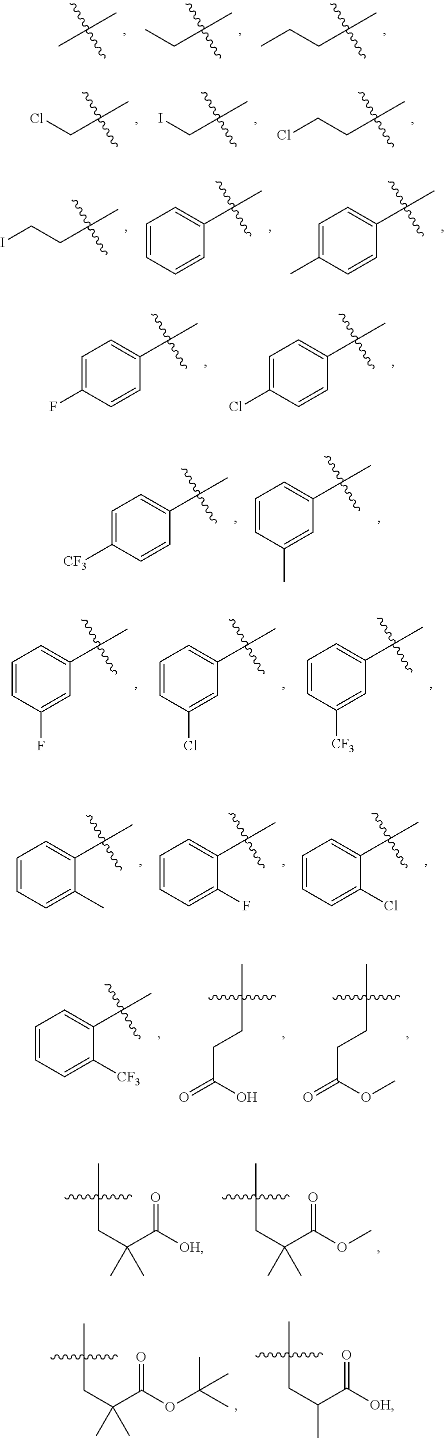 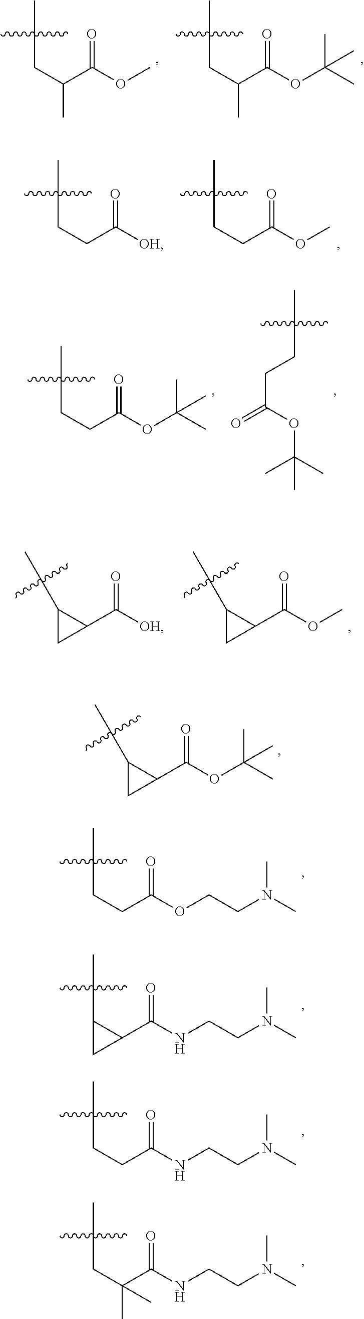 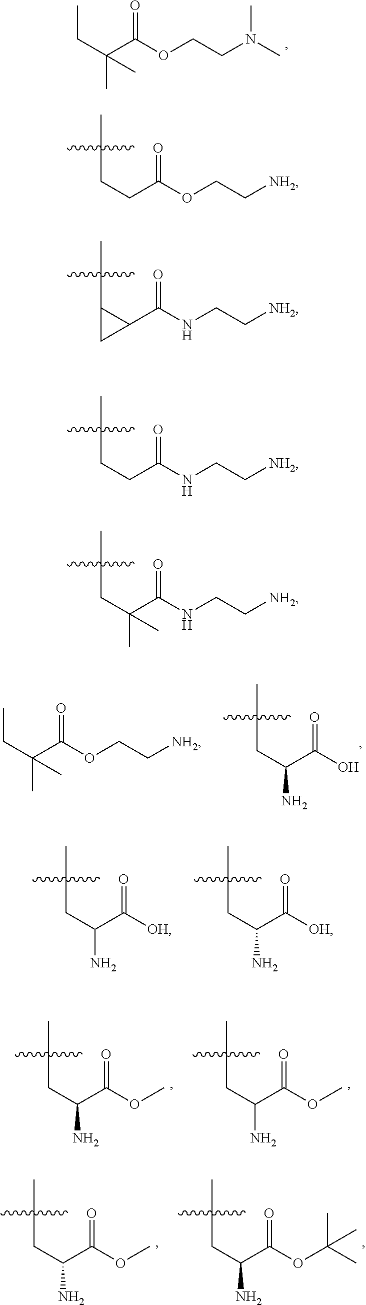 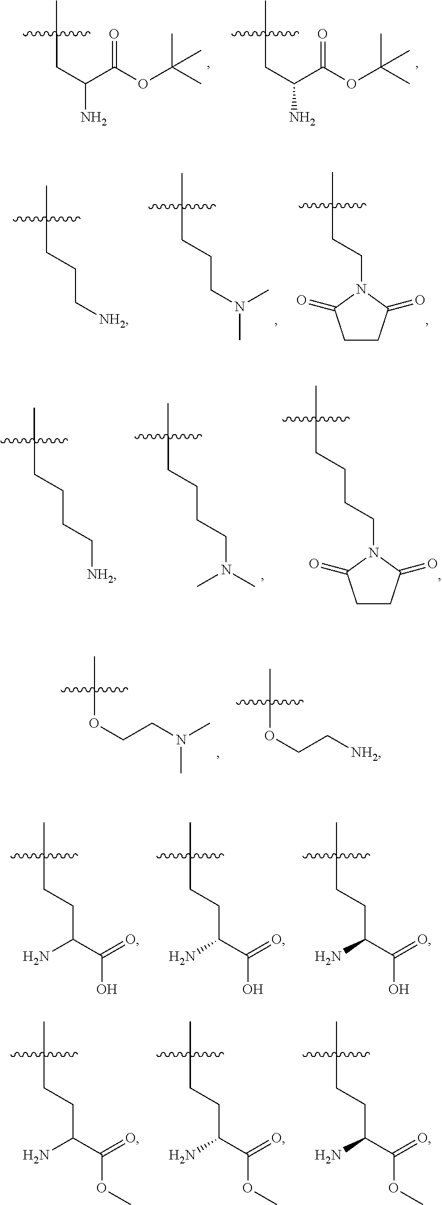 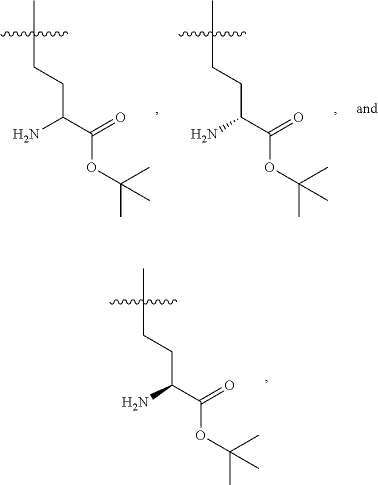
wherein [symbol] denotes the point of attachment of X to Z, or X to Y when Z is absent, in Formula (I); or the point of attachment of the R⁵ group to the carbon atom of the —C(O)— group in Formula (ID).

33. A compound of Formula (IE) 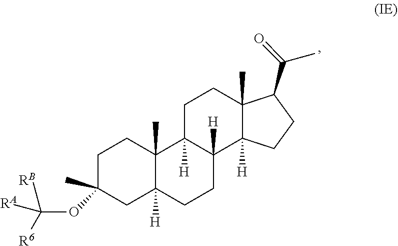
or a pharmaceutically acceptable salt, solvate, hydrate, tautomer, or stereoisomer thereof, wherein
R⁶ is alkyl, alkenyl, alkynyl, heteroalkyl, cycloalkyl, heterocyclyl, aryl, heteroaryl, haloalkyl, alkylene-cycloalkyl, alkylene-heterocyclyl, alkylene-aryl, alkylene-heteroaryl, heteroalkylene-cycloalkyl, heteroalkylene-heterocyclyl, heteroalkylene-aryl, heteroalkylene-heteroaryl, —OC(O)-alkyl, —C(O)O-alkyl, —OC(O)-heteroalkyl, —C(O)O-heteroalkyl, —N₃, —SH, or —S-alkyl (e.g., —S-Me), wherein the alkyl, alkylene, alkenyl, alkynyl, heteroalkyl, heteroalkylene, cycloalkyl, heterocyclyl, aryl, or heteroaryl is optionally substituted; and
R^{A} and R^{B} are each independently hydrogen, alkyl, heteroalkyl, cycloalkyl, heterocyclyl, aryl, or heteroaryl, wherein the alkyl, heteroalkyl, cycloalkyl, heterocyclyl, aryl, or heteroaryl is optionally substituted.

34. The compound of claim 33, wherein R⁶ is a moiety selected from the group consisting of 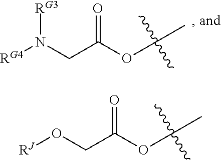
wherein
R^{G3} and R^{G4} are each independently hydrogen, alkyl, or heteroalkyl, wherein the alkyl or heteroalkyl is optionally substituted;
or R^{G3} and R^{G4}, together with the atoms to which they are attached, form a 3-7 membered cycloalkyl, heterocyclyl, aryl, or heteroaryl, wherein the cycloalkyl, heterocyclyl, aryl, or heteroaryl is optionally substituted;
R^{J} is hydrogen, alkyl, alkylene-heterocyclyl, heteroalkyl, or heterocyclyl, wherein the alkyl, alkylene, heteroalkyl, or heterocyclyl is optionally substituted;
and [symbol] denotes the point of attachment of the R⁶ group to the carbon atom of the —C(R^{A})(R^{B})— group.

35. The compound of any one of claims 1, 2, 33, or 34, wherein the X group of Formula (I), or the R⁶ group of Formula (IE), is a moiety selected from the group consisting of 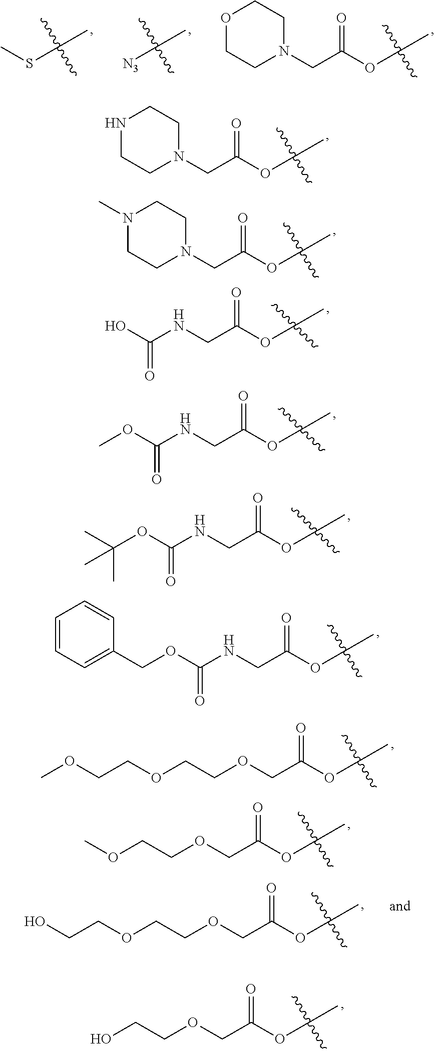
wherein [symbol] denotes the point of attachment of X to Z, or X to Y when Z is absent, in Formula (I); or the point of attachment of the R⁶ group to the carbon atom of the —C(R^{A})(R^{B})— group.

36. The compound of any one of the preceding claims, wherein the compound is a compound selected from Table 1.

37. The compound of any one of claims 1-26 or 36, wherein the compound is selected from the group consisting of 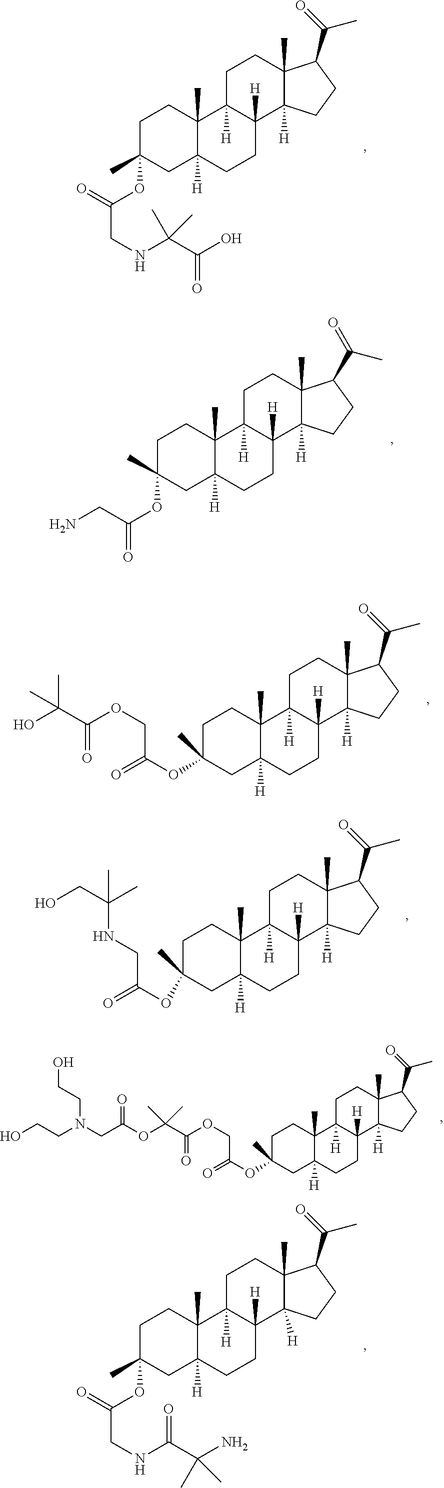
and pharmaceutically acceptable salts, solvates, hydrates, tautomers, and stereoisomers thereof.

38. The compound of any one of claims 1-26 or 36, wherein the compound is selected from the group consisting of 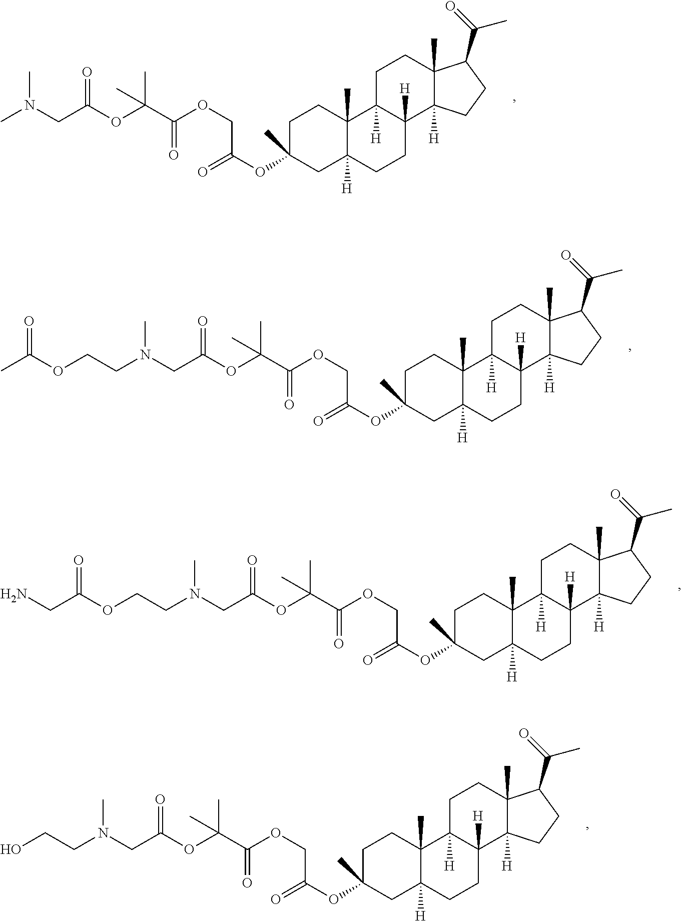
and pharmaceutically acceptable salts, solvates, hydrates, tautomers, and stereoisomers thereof.

39. The compound of any one of claims 1-4, 6, 13, 14, 16, 36, or 37, wherein the compound is 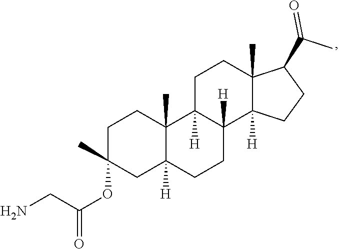
or a pharmaceutically acceptable salt, solvate, hydrate, tautomer, or stereoisomer thereof.

40. The compound of any one of claims 1-38, wherein the compound is not 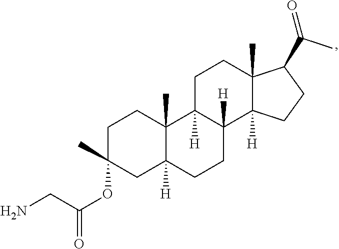
or a pharmaceutically acceptable salt, solvate, hydrate, tautomer, or stereoisomer thereof.

41. The compound of any one of the preceding claims, wherein the compound has a higher aqueous solubility and or hydrophilicity relative to ganaxolone, e.g., as determined by logS, logP, logD, topological polar surface area (TPSA), acid pKa, base pKa, or a combination thereof.

42. The compound of any one of the preceding claims, wherein the compound is substantially converted to ganaxolone by a metabolic process, e.g., following administration to a subject.

43. The compound of any one of the preceding claims, wherein the compound has a higher bioavailability of ganaxolone, relative to ganaxolone delivered by the same route of administration at an equivalent amount, e.g., as determined by serum concentration of ganaxolone (e.g., Cₘₐₓ or AUC).

44. The compound of any one of the preceding claims, wherein the compound provides higher serum concentrations of ganaxolone (e.g., Cₘₐₓ or AUC) and/or a longer half-life of ganaxolone, e.g., following administration to a subject, relative to ganaxolone delivered by the same route of administration at an equivalent amount.

45. A pharmaceutical composition, comprising a compound of any one of the preceding claims, and a pharmaceutically acceptable excipient.

46. A method of treating a disease or disorder in a subject, comprising administering to a subject in need thereof a compound of any one of claims 1-44, or a pharmaceutical composition of claim 45.

47. The method of claim 46, wherein the administering comprises oral administration or intravenous administration.

48. A composition for use in treating a disease or disorder in a subject, comprising a therapeutically effective amount of a compound of any one of claims 1-44, or a pharmaceutical composition of claim 45.

49. Use of a compound of any one of claims 1-44, or a pharmaceutical composition of claim 45, for the production of a medicament effective for treating a disease or disorder in a subject.

50. The method of claim 46 or 47, the composition for use of claim 48, or the use of claim 49, wherein the disease or disorder is a neurological disorder.

51. The method of claim 46 or 47, the composition for use of claim 48, or the use of claim 49, wherein the disease or disorder is a seizure disorder, an epilepsy disorder, a genetic epilepsy disorder, an epilepsy-related disorder, a central nervous system disorder, a neurological disorder, or a neurodegenerative disorder.

52. The method of claim 46 or 47, the composition for use of claim 48, or the use of claim 49, wherein the disease or disorder is status epilepticus (SE).

53. The method of claim 46 or 47, the composition for use of claim 48, or the use of claim 49, wherein the disease or disorder is CDKL5 Deficiency Disorder.

54. The method of claim 46 or 47, the composition for use of claim 48, or the use of claim 49, wherein the disease or disorder is Tuberous Sclerosis Complex.

55. The method of claim 46 or 47, the composition for use of claim 48, or the use of claim 49, wherein the disease or disorder is PCDH19-related epilepsy.

56. The method of claim 46 or 47, the composition for use of claim 48, or the use of claim 49, wherein the disease or disorder is Lennox-Gastaut syndrome.
